(19) United States
(12) Patent Application Publication
(10) Pub. No.: US 2023/0220470 A1
(43) Pub. Date: Jul. 13, 2023

(54) METHODS AND SYSTEMS FOR ANALYZING TARGETABLE PATHOLOGIC PROCESSES IN COVID-19 VIA GENE EXPRESSION ANALYSIS

(71) Applicant: AMPEL BioSolutions, LLC, Charlottesville, VA (US)

(72) Inventors: Andrea DAAMEN, Charlottesville, VA (US); Kathryn K. ALLISON, Matthews, NC (US); Erika HUBBARD, Charlottesville, VA (US); Katherine A. OWEN, Crozet, VA (US); Amrie C. GRAMMER, Charlottesville, VA (US); Peter E. LIPSKY, Charlottesville, VA (US); Robert ROBL, Charlottesville, VA (US)

(21) Appl. No.: 17/924,107

(22) PCT Filed: May 10, 2021
(86) PCT No.: PCT/US2021/031535

§ 371 Date: Nov. 8, 2022

Related U.S. Application Data
(60) Provisional application No. 63/023,088, filed on May 11, 2020.

(51) Int. Cl. C12Q 1/6883; G16B 40/30; G16B 25/10; G16H 50/80

(52) U.S. Cl. CPC C12Q 1/6883; G16B 40/30; G16B 25/10; G16H 50/80; C12Q 2600/158

(57) ABSTRACT

The present disclosure provides systems and methods for machine learning classification and assessment of COVID-19 disease based on gene expression data. In an aspect, a method for determining a COVID-19 disease state of a subject may comprise: (a) assaying a biological sample obtained or derived from the subject to produce a data set comprising gene expression measurements of the biological sample at each of a plurality of COVID-19 disease-associated genomic loci; (b) computer processing the data set to determine the COVID-19 disease state of the subject; and (c) electronically outputting a report indicative of the COVID-19 disease state of the subject.

### CROSS-REFERENCE

This application claims the benefit of U.S. Provisional Patent Application No. 63/023,088, filed May 11, 2020, which is incorporated by reference herein in its entirety.

### BACKGROUND

SARS-CoV2 is a coronavirus and causative agent of the COVID-19 pandemic. COVID-19 typically causes mild respiratory symptoms, but may escalate to acute respiratory distress syndrome (ARDs) with an increased risk of respiratory failure and death. However, the trajectory of disease progression and the status of affected tissues in COVID-19 patients has not been elucidated. A comprehensive analysis of gene expression data from the blood, lung, and airway of COVID-19 patients is performed to investigate the host response to SARS-CoV2 infection. Results indicate that COVID-19 pathogenesis is driven by populations of myeloid-lineage cells with highly inflammatory but distinct transcriptional signatures, suggesting a progression in activation from the periphery to the lung tissue. In addition, through analysis of immune cells and inflammatory pathways enriched in each compartment, a model of the systemic response to SARS-CoV2 is constructed, and therapeutics targeting key upstream regulators of pathways contributing to COVID-19 pathogenesis are identified.

### SUMMARY

In an aspect, the present disclosure provides a method of identifying one or more records having a specific phenotype, the method comprising: receiving a plurality of first records, wherein each first record is associated with one or more of a plurality of phenotypes; receiving a plurality of second records, wherein each second record is associated with one or more of the plurality of phenotypes, and wherein the plurality of second records and the plurality of first records are non-overlapping; applying a machine learning algorithm to at least one first record and at least one second record to determine a classifier; receiving a plurality of third records, wherein the third records are distinct from the plurality of first records and the plurality of second records; and applying the classifier to the plurality of third records to identify one or more third records associated with the specific phenotype.

In some embodiments, the first records and the second records comprise nucleic acid sequencing data, transcriptome data, genome data, epigenome data, proteome data, metabolome data, virome data, metabolome data, methylome data, lipidomic data, lineage-ome data, nucleosomal occupancy data, a genetic variant, a gene fusion, an insertion or deletion (indel), or any combination thereof. In some embodiments, the first records and the second records are in different formats. In some embodiments, the first records and the second records are from different sources, different studies, or both. In some embodiments, the phenotype comprises a disease state, an organ involvement, a medication response, or any combination thereof. In some embodiments, the classifier comprises an elastic generalized linear model classifier, a k-nearest neighbors classifier, a random forest classifier, or any combination thereof.

In some embodiments, the elastic generalized linear model classifier employs an elastic penalty of about 0.8 to about 1. In some embodiments, the elastic generalized linear model classifier employs an elastic penalty of at least about 0.8, about 0.825, about 0.85, about 0.875, about 0.9, about 0.925, about 0.95, about 0.975, or about 1. In some embodiments, the elastic generalized linear model classifier employs an elastic penalty of at most about 0.8, about 0.825, about 0.85, about 0.875, about 0.9, about 0.925, about 0.95, about 0.975, or about 1. In some embodiments, the elastic generalized linear model classifier employs an elastic penalty of about 0.8 to about 0.825, about 0.8 to about 0.85, about 0.8 to about 0.875, about 0.8 to about 0.9, about 0.8 to about 0.925, about 0.8 to about 0.95, about 0.8 to about 0.975, about 0.8 to about 1, about 0.825 to about 0.85, about 0.825 to about 0.875, about 0.825 to about 0.9, about 0.825 to about 0.925, about 0.825 to about 0.95, about 0.825 to about 0.975, about 0.825 to about 1, about 0.85 to about 0.875, about 0.85 to about 0.9, about 0.85 to about 0.925, about 0.85 to about 0.95, about 0.85 to about 0.975, about 0.85 to about 1, about 0.875 to about 0.9, about 0.875 to about 0.925, about 0.875 to about 0.95, about 0.875 to about 0.975, about 0.875 to about 1, about 0.9 to about 0.925, about 0.9 to about 0.95, about 0.9 to about 0.975, about 0.9 to about 1, about 0.925 to about 0.95, about 0.925 to about 0.975, about 0.925 to about 1, about 0.95 to about 0.975, about 0.95 to about 1, or about 0.975 to about 1. In some embodiments, the elastic generalized linear model classifier employs an elastic penalty of about 0.8, about 0.825, about 0.85, about 0.875, about 0.9, about 0.925, about 0.95, about 0.975, or about 1.

In some embodiments, the k-nearest neighbors classifier employs a K value of the size of the plurality of distinct first data sets, wherein k is about 1 to about 20. In some embodiments, the k-nearest neighbors classifier employs a K value of the size of the plurality of distinct first data sets, wherein k is at least about 1, about 2, about 3, about 4, about 5, about 6, about 8, about 10, about 12, about 14, about 16, or about 20. In some embodiments, the k-nearest neighbors classifier employs a K value of the size of the plurality of distinct first data sets, wherein k is at most about 1, about 2, about 3, about 4, about 5, about 6, about 8, about 10, about 12, about 14, about 16, or about 20. In some embodiments, the k-nearest neighbors classifier employs a K value of the size of the plurality of distinct first data sets, wherein k is about 1 to about 2, about 1 to about 3, about 1 to about 4, about 1 to about 5, about 1 to about 6, about 1 to about 8, about 1 to about 10, about 1 to about 12, about 1 to about 14, about 1 to about 16, about 1 to about 20, about 2 to about 3, about 2 to about 4, about 2 to about 5, about 2 to about 6, about 2 to about 8, about 2 to about 10, about 2 to about 12, about 2 to about 14, about 2 to about 16, about 2 to about 20, about 3 to about 4, about 3 to about 5, about 3 to about 6, about 3 to about 8, about 3 to about 10, about 3 to about 12, about 3 to about 14, about 3 to about 16, about 3 to about 20, about 4 to about 5, about 4 to about 6, about 4 to about 8, about 4 to about 10, about 4 to about 12, about 4 to about 14, about 4 to about 16, about 4 to about 20, about 5 to about 6, about 5 to about 8, about 5 to about 10, about 5 to about 12, about 5 to about 14, about 5 to about 16, about 5 to about 20, about 6 to about 8, about 6 to about 10, about 6 to about 12, about 6 to about 14, about 6 to about 16, about 6 to about 20, about 8 to about 10, about 8 to about 12, about 8 to about 14, about 8 to about 16, about 8 to about 20, about 10 to about 12, about 10 to about 14, about 10 to about 16, about 10 to about 20, about 12 to about 14, about 12 to about 16, about 12 to about 20, about 14 to about 16, about 14 to about 20, or about 16 to about 20. In some embodiments, the k-nearest neighbors classifier employs a K value of the size of the plurality of distinct first data sets, wherein k is about 1, about 2, about 3, about 4, about 5, about 6, about 8, about 10, about 12, about 14, about 16, or about 20.

In some embodiments, the K-value of the random forest classifier is incremented by 1 if the k-value is an even number. In some embodiments, applying a machine learning algorithm to the third data set comprises applying a machine learning algorithm to a plurality of unique third data sets.

In some embodiments, the classifier identifies said one or more third records associated with the specific phenotype with an accuracy of about 70% to about 100%. In some embodiments, the classifier identifies said one or more third records associated with the specific phenotype with an accuracy of at least about 70%, about 75%, about 80%, about 85%, about 90%, about 95%, or about 100%. In some embodiments, the classifier identifies said one or more third records associated with the specific phenotype with an accuracy of at most about 70%, about 75%, about 80%, about 85%, about 90%, about 95%, or about 100%. In some embodiments, the classifier identifies said one or more third records associated with the specific phenotype with an accuracy of about 70% to about 75%, about 70% to about 80%, about 70% to about 85%, about 70% to about 90%, about 70% to about 95%, about 70% to about 100%, about 75% to about 80%, about 75% to about 85%, about 75% to about 90%, about 75% to about 95%, about 75% to about 100%, about 80% to about 85%, about 80% to about 90%, about 80% to about 95%, about 80% to about 100%, about 85% to about 90%, about 85% to about 95%, about 85% to about 100%, about 90% to about 95%, about 90% to about 100%, or about 95% to about 100%. In some embodiments, the classifier identifies said one or more third records associated with the specific phenotype with an accuracy of about 70%, about 75%, about 80%, about 85%, about 90%, about 95%, or about 100%.

In some embodiments, the classifier identifies said one or more third records associated with the specific phenotype with an accuracy of about 70% to about 100%. In some embodiments, the classifier identifies said one or more third records associated with the specific phenotype with an accuracy of at least about 70%, about 75%, about 80%, about 85%, about 90%, about 95%, or about 100%. In some embodiments, the classifier identifies said one or more third records associated with the specific phenotype with an accuracy of at most about 70%, about 75%, about 80%, about 85%, about 90%, about 95%, or about 100%. In some embodiments, the classifier identifies said one or more third records associated with the specific phenotype with an accuracy of about 70% to about 75%, about 70% to about 80%, about 70% to about 85%, about 70% to about 90%, about 70% to about 95%, about 70% to about 100%, about 75% to about 80%, about 75% to about 85%, about 75% to about 90%, about 75% to about 95%, about 75% to about 100%, about 80% to about 85%, about 80% to about 90%, about 80% to about 95%, about 80% to about 100%, about 85% to about 90%, about 85% to about 95%, about 85% to about 100%, about 90% to about 95%, about 90% to about 100%, or about 95% to about 100%. In some embodiments, the classifier identifies said one or more third records associated with the specific phenotype with an accuracy of about 70%, about 75%, about 80%, about 85%, about 90%, about 95%, or about 100%.

In some embodiments, the classifier herein enables a specific phenotype association sensitivity of about 70% to about 100%. In some embodiments, the classifier herein enables a specific phenotype association sensitivity of at least 70%, about 75%, about 80%, about 85%, about 90%, about 95%, or about 100%. In some embodiments, the classifier herein enables a specific phenotype association sensitivity of at most 70%, about 75%, about 80%, about 85%, about 90%, about 95%, or about 100%. In some embodiments, the classifier herein enables a specific phenotype association sensitivity of about 70% to about 75%, about 70% to about 80%, about 70% to about 85%, about 70% to about 90%, about 70% to about 95%, about 70% to about 100%, about 75% to about 80%, about 75% to about 85%, about 75% to about 90%, about 75% to about 95%, about 75% to about 100%, about 80% to about 85%, about 80% to about 90%, about 80% to about 95%, about 80% to about 100%, about 85% to about 90%, about 85% to about 95%, about 85% to about 100%, about 90% to about 95%, about 90% to about 100%, or about 95% to about 100%. In some embodiments, the classifier herein enables a specific phenotype association sensitivity of about 70%, about 75%, about 80%, about 85%, about 90%, about 95%, or about 100%.

In some embodiments, the classifier herein enables a specific phenotype association specificity of about 70% to about 100%. In some embodiments, the classifier herein enables a specific phenotype association specificity of at least 70%, about 75%, about 80%, about 85%, about 90%, about 95%, or about 100%. In some embodiments, the classifier herein enables a specific phenotype association specificity of at most 70%, about 75%, about 80%, about 85%, about 90%, about 95%, or about 100%. In some embodiments, the classifier herein enables a specific phenotype association specificity of about 70% to about 75%, about 70% to about 80%, about 70% to about 85%, about 70% to about 90%, about 70% to about 95%, about 70% to about 100%, about 75% to about 80%, about 75% to about 85%, about 75% to about 90%, about 75% to about 95%, about 75% to about 100%, about 80% to about 85%, about 80% to about 90%, about 80% to about 95%, about 80% to about 100%, about 85% to about 90%, about 85% to about 95%, about 85% to about 100%, about 90% to about 95%, about 90% to about 100%, or about 95% to about 100%. In some embodiments, the classifier herein enables a specific phenotype association specificity of about 70%, about 75%, about 80%, about 85%, about 90%, about 95%, or about 100%.

In some embodiments, the method further comprises filtering the first records, the second records, or both. In some embodiments, the filtering comprises removing outliers, removing background noise, removing data without annotation data, normalizing, scaling, variance correcting, Weighted Gene Co-expression Network Analysis, enrichment analysis, dimensionality reduction, or any combination thereof. In some embodiments, the normalizing is performed by Robust Multi-Array Analysis (RMA), Guanine Cytosine Robust Multi-Array Analysis (GCRMA), Linear Models for Microarray Data, variance stabilizing transformation (VST), normal-exponential quantile correction (NEQC), or any combination thereof. In some embodiments, the variance correction comprises employing a local empirical Bayesian shrinkage, adjusting the p-values for multiple hypothesis testing using the Benjamini-Hochberg correction, and removing all data with a set false discovery rate

In some embodiments, the false discovery rate is about 0.000001 to about 0.2. In some embodiments, the false discovery rate is at least about 0.000001. In some embodiments, the false discovery rate is at most about 0.2. In some embodiments, the false discovery rate is about 0.000001 to about 0.00005, about 0.000001 to about 0.00001, about 0.000001 to about 0.0005, about 0.000001 to about 0.0001, about 0.000001 to about 0.005, about 0.000001 to about 0.001, about 0.000001 to about 0.05, about 0.000001 to about 0.01, about 0.000001 to about 0.2, about 0.00005 to about 0.00001, about 0.00005 to about 0.0005, about 0.00005 to about 0.0001, about 0.00005 to about 0.005, about 0.00005 to about 0.001, about 0.00005 to about 0.05, about 0.00005 to about 0.01, about 0.00005 to about 0.2, about 0.00001 to about 0.0005, about 0.00001 to about 0.0001, about 0.00001 to about 0.005, about 0.00001 to about 0.001, about 0.00001 to about 0.05, about 0.00001 to about 0.01, about 0.00001 to about 0.2, about 0.0005 to about 0.0001, about 0.0005 to about 0.005, about 0.0005 to about 0.001, about 0.0005 to about 0.05, about 0.0005 to about 0.01, about 0.0005 to about 0.2, about 0.0001 to about 0.005, about 0.0001 to about 0.001, about 0.0001 to about 0.05, about 0.0001 to about 0.01, about 0.0001 to about 0.2, about 0.005 to about 0.001, about 0.005 to about 0.05, about 0.005 to about 0.01, about 0.005 to about 0.2, about 0.001 to about 0.05, about 0.001 to about 0.01, about 0.001 to about 0.2, about 0.05 to about 0.01, about 0.05 to about 0.2, or about 0.01 to about 0.2. In some embodiments, the false discovery rate is about 0.000001, about 0.00005, about 0.00001, about 0.0005, about 0.0001, about 0.005, about 0.001, about 0.05, about 0.01, or about 0.2.

In some embodiments, the Weighted Gene Co-expression Network Analysis comprises calculating a topology matrix, clustering the data based on the topology matrix, and correlating module eigenvalues for traits on a linear scale by Pearson correlation, for nonparametric traits by Spearman correlation, and for dichotomous traits by point-biserial correlation or t-test. The Pearson correlation or the Product Moment Correlation Coefficient (PMCC), is a number between −1 and 1 that indicates the extent to which two variables are linearly related. The Spearman correlation is a nonparametric measure of rank correlation; statistical dependence between the rankings of two variables.

In some embodiments, the one or more records having a specific phenotype correspond to one or more subjects, and the method further comprises identifying the one or more subjects as (i) having a diagnosis of a disease state or condition, (ii) having a prognosis of a disease state or condition, (iii) being suitable or not suitable for enrollment in a clinical trial for a disease state or condition, (iv) being suitable or not suitable for being administered a therapeutic regimen configured to treat a disease state or condition, (v) having an efficacy or not having an efficacy of a therapeutic regimen configured to treat a disease state or condition, based at least in part on the specific phenotype corresponding to the one or more subjects.

In another aspect, the present disclosure provides a non-transitory computer-readable storage media encoded with a computer program including instructions executable by a processor to create an application for identifying one or more records having a specific phenotype, the application comprising: a first receiving module receiving a plurality of first records, wherein each first record is associated with one or more of a plurality of phenotypes; a second receiving module receiving a plurality of second records, wherein each second record is associated with one or more of the plurality of phenotypes, and wherein the plurality of second records and the plurality of first records are non-overlapping; a machine learning module applying a machine learning algorithm to at least one first record and at least one second record to determine a classifier; a third receiving module receiving a plurality of third records, wherein the third records are distinct from the plurality of first records and the plurality of second records; and a classifying module applying the classifier to the plurality of third records to identify one or more third records associated with the specific phenotype.

In some embodiments, the first records and the second records comprise nucleic acid sequencing data, transcriptome data, genome data, epigenome data, proteome data, metabolome data, virome data, metabolome data, methylome data, lipidomic data, lineage-ome data, nucleosomal occupancy data, a genetic variant, a gene fusion, an insertion or deletion (indel), or any combination thereof. In some embodiments, the first records and the second records are in different formats. In some embodiments, the first records and the second records are from different sources, different studies, or both. In some embodiments, the phenotype comprises a disease state, an organ involvement, a medication response, or any combination thereof. In some embodiments, the classifier comprises an elastic generalized linear model classifier, a k-nearest neighbors classifier, a random forest classifier, or any combination thereof. In some embodiments, the elastic generalized linear model classifier employs an elastic penalty of about 0.9. In some embodiments, the k-nearest neighbors classifier employs a K-value of about 5% of the size of the plurality of distinct first data sets. In some embodiments, the K-value of the random forest classifier is incremented by 1 if the k-value is an even number. In some embodiments, applying a machine learning algorithm to the third data set comprises applying a machine learning algorithm to a plurality of unique third data sets. In some embodiments, said classifier identifies said one or more third records associated with the specific phenotype with an accuracy of at least about 70%. In some embodiments, the method further comprises filtering the first records, the second records, or both. In some embodiments, the filtering comprises removing outliers, removing background noise, removing data without annotation data, normalizing, scaling, variance correcting, Weighted Gene Co-expression Network Analysis, enrichment analysis, dimensionality reduction, or any combination thereof. In some embodiments, the normalizing is performed by Robust Multi-Array Analysis (RMA), Guanine Cytosine Robust Multi-Array Analysis (GCRMA), Linear Models for Microarray Data, variance stabilizing transformation (VST), normal-exponential quantile correction (NEQC), or any combination thereof. In some embodiments, the variance correction comprises employing a local empirical Bayesian shrinkage, adjusting the p-values for multiple hypothesis testing using the Benjamini-Hochberg correction, and removing all data with a false discovery rate of less than 0.2. In some embodiments, the Weighted Gene Co-expression Network Analysis comprises calculating a topology matrix, clustering the data based on the topology matrix, and correlating module eigenvalues for traits on a linear scale by Pearson correlation, for nonparametric traits by Spearman correlation, and for dichotomous traits by point-biserial correlation or t-test.

In another aspect, the present disclosure provides a method for identifying a disease state or a susceptibility thereof of a subject, comprising: (a) using an assay to process a biological sample derived from the subject to generate a quantitative measure of each of a plurality of disease-associated genomic loci; (b) processing the dataset to identify the disease state or the susceptibility thereof of the subject; and (c) electronically outputting a report indicative of the disease state or the susceptibility thereof of the subject.

In some embodiments, the plurality of quantitative measures comprises gene expression measurements. In some embodiments, the disease state comprises an active COVID-19 condition or an inactive COVID-19 condition.

In another aspect, the present disclosure provides a computer-implemented method for assessing a disease state or condition of a subject, comprising: (a) receiving a dataset of a biological sample of the subject; (b) selecting one or more data analysis tools, wherein the one or more data analysis tools comprise an analysis tool selected from the group consisting of: a BIG-C™ big data analysis tool, an I-Scope™ big data analysis tool, a T-Scope™ big data analysis tool, a CellScan big data analysis tool, an MS (Molecular Signature) Scoring™ analysis tool, a Gene Set Variation Analysis (GSVA) tool (e.g., P-Scope), and a combination thereof; (c) processing the dataset using the one or more data analysis tools to generate a data signature of the biological sample of the subject; and (d) based at least in part on the data signature generated in (c), assessing the disease state or condition of the subject.

In some embodiments, the dataset comprises gene expression measurements. In some embodiments, the disease state comprises an active COVID-19 condition or an inactive COVID-19 condition.

In some embodiments, the dataset comprises mRNA gene expression or transcriptome data, DNA genomic data, proteomic data, metabolomic data, or a combination thereof. In some embodiments, the biological sample is selected from the group consisting of: a whole blood (WB) sample, a PBMC sample, a tissue sample, and a cell sample. In some embodiments, assessing the condition of the subject comprises identifying a disease or disorder of the subject.

In some embodiments, the method further comprises identifying a disease or disorder of the subject at a sensitivity or specificity of at least about 70%. In some embodiments, the method further comprises determining a likelihood of the identification of the disease or disorder of the subject. In some embodiments, the method further comprises providing a therapeutic intervention for the disease or disorder of the subject. In some embodiments, the method further comprises monitoring the disease or disorder of the subject, wherein the monitoring comprises assessing the disease or disorder of the subject at a plurality of time points, wherein the assessing is based at least on the disease or disorder identified at each of the plurality of time points.

In some embodiments, selecting the one or more data analysis tools comprises receiving a user selection of the one or more data analysis tools. In some embodiments, selecting the one or more data analysis tools is automatically performed by the computer without receiving a user selection of the one or more data analysis tools.

In another aspect, the present disclosure provides a computer system for assessing a disease state or condition of a subject, comprising: a database that is configured to store a dataset of a biological sample of the subject; and one or more computer processors operatively coupled to the database, wherein the one or more computer processors are individually or collectively programmed to: (i) select one or more data analysis tools, wherein the one or more data analysis tools comprise an analysis tool selected from the group consisting of: a BIG-C™ big data analysis tool, an I-Scope™ big data analysis tool, a T-Scope™ big data analysis tool, a CellScan big data analysis tool, an MS (Molecular Signature) Scoring™ analysis tool, a Gene Set Variation Analysis (GSVA) tool (e.g., P-Scope), and a combination thereof; (ii) process the dataset using the one or more data analysis tools to generate a data signature of the biological sample of the subject; and (iii) based at least in part on the data signature generated in (ii), assess the disease state or condition of the subject.

In some embodiments, the dataset comprises gene expression measurements. In some embodiments, the disease state comprises an active COVID-19 condition or an inactive COVID-19 condition.

In another aspect, the present disclosure provides a non-transitory computer-readable medium comprising machine-executable code that, upon execution by one or more computer processors, implements a method for assessing a disease state or condition of a subject, the method comprising: (a) receiving a dataset of a biological sample of the subject; (b) selecting one or more data analysis tools, wherein the one or more data analysis tools comprise an analysis tool selected from the group consisting of: a BIG-C™ big data analysis tool, an I-Scope™ big data analysis tool, a T-Scope™ big data analysis tool, a CellScan big data analysis tool, an MS (Molecular Signature) Scoring™ analysis tool, a Gene Set Variation Analysis (GSVA) tool (e.g., P-Scope), and a combination thereof (c) processing the dataset using the one or more data analysis tools to generate a data signature of the biological sample of the subject; and (d) based at least in part on the data signature generated in (c), assessing the disease state or condition of the subject.

In some embodiments, the dataset comprises gene expression measurements. In some embodiments, the disease state comprises an active COVID-19 condition or an inactive COVID-19 condition.

In any embodiment described herein, the one or more data analysis tools can be a plurality of data analysis tools each independently selected from a BIG-C™ big data analysis tool, an I-Scope™ big data analysis tool, a T-Scope™ big data analysis tool, a CellScan big data analysis tool, an MS (Molecular Signature) Scoring™ analysis tool, a Gene Set Variation Analysis (GSVA) tool (e.g., P-Scope), and a combination thereof.

In another aspect, the present disclosure provides a method for determining a COVID-19 disease state of a subject, comprising: (a) assaying a biological sample obtained or derived from the subject to produce a data set comprising gene expression measurements of the biological sample at each of a plurality of COVID-19 disease-associated genomic loci, wherein the plurality of COVID-19 disease-associated genomic loci comprises at least a portion of a gene selected from the group of genes listed in Table 6 and Tables 7A-7C; (b) computer processing the data set to determine the COVID-19 disease state of the subject; and (c) electronically outputting a report indicative of the COVID-19 disease state of the subject.

In some embodiments, the plurality of COVID-19 disease-associated genomic loci comprises at least a portion of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 350, 400, 450, 500, 550, 600, 650, 700, 750, 800, 850, 900, 950, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, or 6000 genes selected from the group of genes listed in Table 6 and Tables 7A-7C.

In some embodiments, the method further comprises determining the COVID-19 disease state of the subject with an accuracy of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

In some embodiments, the method further comprises determining the COVID-19 disease state of the subject with a sensitivity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

In some embodiments, the method further comprises determining the COVID-19 disease state of the subject with a specificity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

In some embodiments, the method further comprises determining the COVID-19 disease state of the subject with a positive predictive value of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

In some embodiments, the method further comprises determining the COVID-19 disease state of the subject with a negative predictive value of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

In some embodiments, the method further comprises determining the COVID-19 disease state of the subject with an Area-Under-Curve (AUC) of at least about 0.50, at least about 0.55, at least about 0.60, at least about 0.65, at least about 0.70, at least about 0.75, at least about 0.80, at least about 0.85, at least about 0.90, at least about 0.91, at least about 0.92, at least about 0.93, at least about 0.94, at least about 0.95, at least about 0.96, at least about 0.97, at least about 0.98, at least about 0.99, or more than about 0.99.

In some embodiments, the subject has received a diagnosis of the COVID-19 disease. In some embodiments, the subject is suspected of having the COVID-19 disease. In some embodiments, the subject is at elevated risk of having the COVID-19 disease or having severe complications from the COVID-19 disease. In some embodiments, the subject is asymptomatic for the COVID-19 disease. In some embodiments, the method further comprises administering a treatment to the subject based at least in part on the determined COVID-19 disease state. In some embodiments, the treatment is configured to treat the COVID-19 disease state of the subject. In some embodiments, the treatment is configured to reduce a severity of the COVID-19 disease state of the subject. In some embodiments, the treatment is configured to reduce a risk of having the COVID-19 disease. In some embodiments, the treatment comprises a drug. In some embodiments, the drug is selected from the group listed in Tables 8A-8B.

In some embodiments, (b) comprises using a trained machine learning classifier to analyze the data set to determine the COVID-19 disease state of the subject. In some embodiments, the trained machine learning classifier is trained using gene expression data obtained by a data analysis tool selected from the group consisting of: a BIG-C™ big data analysis tool, an I-Scope™ big data analysis tool, a T-Scope™ big data analysis tool, a CellScan big data analysis tool, an MS (Molecular Signature) Scoring™ analysis tool, and a Gene Set Variation Analysis (GSVA) tool. In some embodiments, the trained machine learning classifier is selected from the group consisting of a linear regression, a logistic regression, a Ridge regression, a Lasso regression, an elastic net (EN) regression, a support vector machine (SVM), a gradient boosted machine (GBM), a k nearest neighbors (kNN), a generalized linear model (GLM), a naïve Bayes (NB) classifier, a neural network, a Random Forest (RF), a deep learning algorithm, and a combination thereof.

In some embodiments, (b) comprises comparing the data set to a reference data set. In some embodiments, the reference data set comprises gene expression measurements of reference biological samples at each of the plurality of COVID-19 disease-associated genomic loci. In some embodiments, the reference biological samples comprise a first plurality of biological samples obtained or derived from subjects having the COVID-19 disease and a second plurality of biological samples obtained or derived from subjects not having the COVID-19 disease.

In some embodiments, the biological sample is selected from the group consisting of: a blood sample, isolated peripheral blood mononuclear cells (PBMCs), a biopsy sample, and any derivative thereof.

In some embodiments, the method further comprises determining a likelihood of the determined COVID-19 disease state.

In some embodiments, the method further comprises monitoring the COVID-19 disease state of the subject, wherein the monitoring comprises assessing the COVID-19 disease state of the subject at a plurality of time points.

In some embodiments, a difference in the assessment of the COVID-19 disease state of the subject among the plurality of time points is indicative of one or more clinical indications selected from the group consisting of: (i) a diagnosis of the COVID-19 disease state of the subject, (ii) a prognosis of the COVID-19 disease state of the subject, and (iii) an efficacy or non-efficacy of a course of treatment for treating the COVID-19 disease state of the subject.

In another aspect, the present disclosure provides a computer system for determining a COVID-19 disease state of a subject, comprising: a database that is configured to store a dataset comprising gene expression data, wherein the gene expression data is obtained by assaying a biological sample obtained or derived from the subject to produce gene expression measurements of the biological sample at each of a plurality of COVID-19 disease-associated genomic loci, wherein the plurality of COVID-19 disease-associated genomic loci comprises at least a portion of a gene selected from the group of genes listed in Table 6 and Tables 7A-7C; and one or more computer processors operatively coupled to the database, wherein the one or more computer processors are individually or collectively programmed to: (i) computer process the data set to determine the COVID-19 disease state of the subject; (ii) electronically output a report indicative of the COVID-19 disease state of the subject.

In some embodiments, the plurality of COVID-19 disease-associated genomic loci comprises at least a portion of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 350, 400, 450, 500, 550, 600, 650, 700, 750, 800, 850, 900, 950, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, or 6000 genes selected from the group of genes listed in Table 6 and Tables 7A-7C.

In some embodiments, the one or more computer processors are individually or collectively programmed to further determine the COVID-19 disease state of the subject with an accuracy of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

In some embodiments, the one or more computer processors are individually or collectively programmed to further determine the COVID-19 disease state of the subject with a sensitivity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

In some embodiments, the one or more computer processors are individually or collectively programmed to further determine the COVID-19 disease state of the subject with a specificity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

In some embodiments, the one or more computer processors are individually or collectively programmed to further determine the COVID-19 disease state of the subject with a positive predictive value of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

In some embodiments, the one or more computer processors are individually or collectively programmed to further determine the COVID-19 disease state of the subject with a negative predictive value of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

In some embodiments, the one or more computer processors are individually or collectively programmed to further determine the COVID-19 disease state of the subject with an Area-Under-Curve (AUC) of at least about 0.50, at least about 0.55, at least about 0.60, at least about 0.65, at least about 0.70, at least about 0.75, at least about 0.80, at least about 0.85, at least about 0.90, at least about 0.91, at least about 0.92, at least about 0.93, at least about 0.94, at least about 0.95, at least about 0.96, at least about 0.97, at least about 0.98, at least about 0.99, or more than about 0.99.

In some embodiments, the subject has received a diagnosis of the COVID-19 disease. In some embodiments, the subject is suspected of having the COVID-19 disease. In some embodiments, the subject is at elevated risk of having the COVID-19 disease or having severe complications from the COVID-19 disease. In some embodiments, the subject is asymptomatic for the COVID-19 disease. In some embodiments, the one or more computer processors are individually or collectively programmed to further direct a treatment to be administered to the subject based at least in part on the determined COVID-19 disease state. In some embodiments, the treatment is configured to treat the COVID-19 disease state of the subject. In some embodiments, the treatment is configured to reduce a severity of the COVID-19 disease state of the subject. In some embodiments, the treatment is configured to reduce a risk of having the COVID-19 disease. In some embodiments, the treatment comprises a drug. In some embodiments, the drug is selected from the group listed in Tables 8A-8B.

In some embodiments, (i) comprises using a trained machine learning classifier to analyze the data set to determine the COVID-19 disease state of the subject. In some embodiments, the trained machine learning classifier is trained using gene expression data obtained by a data analysis tool selected from the group consisting of: a BIG-C™ big data analysis tool, an I-Scope™ big data analysis tool, a T-Scope™ big data analysis tool, a CellScan big data analysis tool, an MS (Molecular Signature) Scoring™ analysis tool, and a Gene Set Variation Analysis (GSVA) tool. In some embodiments, the trained machine learning classifier is selected from the group consisting of a linear regression, a logistic regression, a Ridge regression, a Lasso regression, an elastic net (EN) regression, a support vector machine (SVM), a gradient boosted machine (GBM), a k nearest neighbors (kNN), a generalized linear model (GLM), a naïve Bayes (NB) classifier, a neural network, a Random Forest (RF), a deep learning algorithm, and a combination thereof.

In some embodiments, (i) comprises comparing the data set to a reference data set. In some embodiments, the reference data set comprises gene expression measurements of reference biological samples at each of the plurality of COVID-19 disease-associated genomic loci. In some embodiments, the reference biological samples comprise a first plurality of biological samples obtained or derived from subjects having the COVID-19 disease and a second plurality of biological samples obtained or derived from subjects not having the COVID-19 disease.

In some embodiments, the biological sample is selected from the group consisting of: a blood sample, isolated peripheral blood mononuclear cells (PBMCs), a biopsy sample, and any derivative thereof.

In some embodiments, the one or more computer processors are individually or collectively programmed to further determine a likelihood of the determined COVID-19 disease state.

In some embodiments, the one or more computer processors are individually or collectively programmed to further monitor the COVID-19 disease state of the subject, wherein the monitoring comprises assessing the COVID-19 disease state of the subject at a plurality of time points.

In some embodiments, a difference in the assessment of the COVID-19 disease state of the subject among the plurality of time points is indicative of one or more clinical indications selected from the group consisting of: (i) a diagnosis of the COVID-19 disease state of the subject, (ii) a prognosis of the COVID-19 disease state of the subject, and (iii) an efficacy or non-efficacy of a course of treatment for treating the COVID-19 disease state of the subject.

In another aspect, the present disclosure provides a non-transitory computer readable medium comprising machine-executable code that, upon execution by one or more computer processors, implements a method for determining a COVID-19 disease state of a subject, the method comprising: (a) assaying a biological sample obtained or derived from the subject to produce a data set comprising gene expression measurements of the biological sample at each of a plurality of COVID-19 disease-associated genomic loci, wherein the plurality of COVID-19 disease-associated genomic loci comprises at least a portion of a gene selected from the group of genes listed in Table 6 and Tables 7A-7C; (b) computer processing the data set to determine the COVID-19 disease state of the subject; and (c) electronically outputting a report indicative of the COVID-19 disease state of the subject.

In some embodiments, the plurality of COVID-19 disease-associated genomic loci comprises at least a portion of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 350, 400, 450, 500, 550, 600, 650, 700, 750, 800, 850, 900, 950, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, or 6000 genes selected from the group of genes listed in Table 6 and Tables 7A-7C.

In some embodiments, the method further comprises determining the COVID-19 disease state of the subject with an accuracy of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

In some embodiments, the method further comprises determining the COVID-19 disease state of the subject with a sensitivity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

In some embodiments, the method further comprises determining the COVID-19 disease state of the subject with a specificity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

In some embodiments, the method further comprises determining the COVID-19 disease state of the subject with a positive predictive value of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

In some embodiments, the method further comprises determining the COVID-19 disease state of the subject with a negative predictive value of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

In some embodiments, the method further comprises determining the COVID-19 disease state of the subject with an Area-Under-Curve (AUC) of at least about 0.50, at least about 0.55, at least about 0.60, at least about 0.65, at least about 0.70, at least about 0.75, at least about 0.80, at least about 0.85, at least about 0.90, at least about 0.91, at least about 0.92, at least about 0.93, at least about 0.94, at least about 0.95, at least about 0.96, at least about 0.97, at least about 0.98, at least about 0.99, or more than about 0.99.

In some embodiments, the subject has received a diagnosis of the COVID-19 disease. In some embodiments, the subject is suspected of having the COVID-19 disease. In some embodiments, the subject is at elevated risk of having the COVID-19 disease or having severe complications from the COVID-19 disease. In some embodiments, the subject is asymptomatic for the COVID-19 disease. In some embodiments, the method further comprises administering a treatment to the subject based at least in part on the determined COVID-19 disease state. In some embodiments, the treatment is configured to treat the COVID-19 disease state of the subject. In some embodiments, the treatment is configured to reduce a severity of the COVID-19 disease state of the subject. In some embodiments, the treatment is configured to reduce a risk of having the COVID-19 disease. In some embodiments, the treatment comprises a drug. In some embodiments, the drug is selected from the group listed in Tables 8A-8B.

In some embodiments, (b) comprises using a trained machine learning classifier to analyze the data set to determine the COVID-19 disease state of the subject. In some embodiments, the trained machine learning classifier is trained using gene expression data obtained by a data analysis tool selected from the group consisting of: a BIG-C™ big data analysis tool, an I-Scope™ big data analysis tool, a T-Scope™ big data analysis tool, a CellScan big data analysis tool, an MS (Molecular Signature) Scoring™ analysis tool, and a Gene Set Variation Analysis (GSVA) tool. In some embodiments, the trained machine learning classifier is selected from the group consisting of a linear regression, a logistic regression, a Ridge regression, a Lasso regression, an elastic net (EN) regression, a support vector machine (SVM), a gradient boosted machine (GBM), a k nearest neighbors (kNN), a generalized linear model (GLM), a naïve Bayes (NB) classifier, a neural network, a Random Forest (RF), a deep learning algorithm, and a combination thereof.

In some embodiments, (b) comprises comparing the data set to a reference data set. In some embodiments, the reference data set comprises gene expression measurements of reference biological samples at each of the plurality of COVID-19 disease-associated genomic loci. In some embodiments, the reference biological samples comprise a first plurality of biological samples obtained or derived from subjects having the COVID-19 disease and a second plurality of biological samples obtained or derived from subjects not having the COVID-19 disease.

In some embodiments, the biological sample is selected from the group consisting of: a blood sample, isolated peripheral blood mononuclear cells (PBMCs), a biopsy sample, and any derivative thereof.

In some embodiments, the method further comprises determining a likelihood of the determined COVID-19 disease state.

In some embodiments, the method further comprises monitoring the COVID-19 disease state of the subject, wherein the monitoring comprises assessing the COVID-19 disease state of the subject at a plurality of time points.

In some embodiments, a difference in the assessment of the COVID-19 disease state of the subject among the plurality of time points is indicative of one or more clinical indications selected from the group consisting of: (i) a diagnosis of the COVID-19 disease state of the subject, (ii) a prognosis of the COVID-19 disease state of the subject, and (iii) an efficacy or non-efficacy of a course of treatment for treating the COVID-19 disease state of the subject.

Another aspect of the present disclosure provides a non-transitory computer-readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

The patent application file contains at least one drawing executed in color. Copies of this patent application publication with color drawing(s) will be provided by the Office upon request and payment of the necessary fee.

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A shows transcriptional changes in the progression of the pathologic response to SARS-CoV2 traced through three compartments (blood, lung, and airway) from activation and mobilization of immune cells in the blood, to infiltration into the lung tissue and airway of infected patients.

FIGS. 1B-1D show differences in inflammatory pathways and immune cell types enriched in COVID-19 patients as compared to healthy controls as well those that were differentially enriched between the blood, lung, and airway compartments, including increased inflammatory pathway signatures (FIG. 1B), decreased lymphoid cell signatures (FIG. 1C), and increased myeloid cell signatures (FIG. 1D) in COVID-19 patients.

FIGS. 2A-2F show differential expression of specific genes of interest (FIGS. 2A-2B); GSVA enrichment of non-hematopoietic cell type gene signatures including fibroblasts, Type I and Type II alveolar cells, ciliated lung cells, club cells, and a general lung tissue cell signature (FIGS. 2C-2E); and increased expression of the viral entry genes ACE2 and TMPRSS2, which are typically expressed on lung epithelium (ref), in the airway of SARS-CoV2-infected patients (FIG. 2F).

FIGS. 3A-3F show that PBMC cluster 8 was dominated by an inflammatory monocyte population defined by C2, C5, CXCL10, CCR2 and multiple interferon-stimulated genes, whereas cluster 3 contained hallmarks of alternatively activated (M2) macrophages and/or myeloid-derived suppressor cells (MDSCs), including CD33, CD36, CD93 and ITGAM (FIG. 3A; similar to the blood, lung-derived monocyte/myeloid genes segregated into clusters associated with common myeloid-lineage cell functions, such as chemotaxis and pattern recognition, as well as multiple subpopulations (FIG. 3E);

FIGS. 4A-4D show GSVA evaluation of both monocyte cell surface and monocyte secreted gene expression confirmed heterogeneous cell surface markers in the BAL, and increased chemokine secretion in the lung (FIG. 4A); Each cluster was evaluated in its respective tissue sample and control by GSVA (FIG. 4B); comparison of the co-expressed genes between each tissue myeloid population shows that many of the increased genes in each tissue are shared (FIG. 4C); and significant overlap, as determined by Fisher's Exact Test, in many populations (FIG. 4D).

FIGS. 5A-5E show myeloid cell population in the PBMCs was found to be highly glycolytic, whereas there was no significant change to metabolism detected in the lung, and the population in the BAL was reliant on OXPHOS (FIG. 5A); although myeloid cell signatures were associated with the NLRP3 inflammasome in the PBMCs and lungs, this association was not found in the BAL (FIG. 5B); the classical complement cascade was significantly correlated with the increased myeloid cells in both PBMCs and BAL, whereas the alternative complement cascade was significantly correlated with the myeloid cells in the lung (FIG. 5C); the myeloid cells in the PBMCs were also significantly correlated with the cell cycle, but this may be more evident of plasma cells in the blood (FIG. 5D); additionally, the lung and BAL myeloid populations were negatively correlated with apoptosis (FIG. 5E).

FIGS. 6A-6B show results from pathway analysis on DEGs from each of the peripheral blood, lung, and airway compartments using IPA canonical signaling pathway and upstream regulator analysis functions, including that interferon signaling, the inflammasome, and other components of antiviral, innate immunity were reflected by the disease state gene expression profile compared to healthy controls (FIG. 6A); and that upstream regulators predicted to mediate responses to the virus in each compartment indicated uniform involvement of proinflammatory cytokines with type I interferon regulation dominant in the diseased lung (FIG. 6B).

FIG. 7 shows that by comparing DE results from multiple compartments (the blood, lung, and airway) in COVID-19 patients, we have developed a model of the systemic pathogenic response to SARS-CoV2 infection.

FIGS. 8A-8C show previously defined gene modules characterizing immune and inflammatory cells and processes.

FIGS. 9A-9B show increased expression of Type I interferon genes (IFNA4, IFNA6, IFNA10) and significant enrichment of the Type I and Type II IGS specifically in the lung tissue, but not in the blood or airway of COVID-19 patients.

FIGS. 10A-10C show GSVA enrichment of non-hematopoietic cell type gene signatures including fibroblasts, Type I and Type II alveolar cells, ciliated lung cells, club cells, and a general lung tissue cell signature.

FIGS. 11A-11D show that peripheral blood exhibited profoundly suppressed T cells determined by the downregulation of T cell activation markers CD28, LCK and ITK (FIG. 11A); metabolic function in the lung was varied, however, upregulated genes segregated with glycolysis, potentially reflecting cellular activation (cluster 18), whereas OXPHOS was predominantly downregulated along with other nuclear processes (transcription and mRNA processing) (FIG. 11B); similar to the PBMC compartment, T cells were decreased in the airway (FIGS. 11A and 11C); non-hematopoietic cell signatures in the BAL were similar in content to those derived from in vitro COVID19-infected lung epithelium primary cell lines (NHBE) REF (FIG. 11D);

FIGS. 12A-12F show upregulation of FCN1 (cluster 15), SELL (cluster 14) and S100A8/A9 (cluster 4) which comprise an inflammatory monocyte signature (G1 population) derived from the BAL fluid of COVID patients recently described by Liao et al. (FIG. 12A); the G1 and G2 population genes, characterized by predominately interferon-stimulated genes, were increased in the lung (FIG. 12B); conversely, the “novel intermediate macrophage” population (G2) characterized by inflammatory mediators and chemokines such as CCL2, CCL3 and CCL4, was increased in the BAL, but not the PBMCs (FIG. 12C); additionally, per patient analyses confirmed the presence of the “pro-fibrotic, SPP1+” macrophage (G3) and “lung alveolar macrophage” populations (G4) in the BAL, although there also may be some evidence of these populations in the lung (FIGS. 12D-12E); evidence of recently described alveolar macrophages (AMs; G4 population) specifically in the airway (Liao et al., 2020), although these markers were distributed among multiple clusters, including FABP4 and PPARG in cluster 17, SPP1 and MRC1 in cluster 10, and MARCO and TFRC in clusters 34 and 7, respectively (FIG. 12E); and comparison of this population with additional previously defined myeloid populations demonstrated significant overlap with IFN-activated macrophages, CX3CR1+ lining macrophages (arthritis model) and an alveolar macrophage phenotype (FIG. 12F).

FIGS. 13A-13C show that DE interrogation of all possible myeloid cell-specific genes demonstrated further heterogeneity in expression of markers, such as CD14, CD300C, and OSCAR between compartments.

FIG. 14 shows that for each gene, a Pearson correlation coefficient was calculated with every other myeloid cell gene for both the samples and controls in each tissue compartment; and the resultant correlation coefficient matrices were then hierarchically clustered into two clusters based upon co-expression.

FIGS. 15A-15E show results from evaluating metabolism in each compartment using GSVA, including that the TCA cycle was significantly increased in PBMCs, whereas OXPHOS is significantly increased in the BAL (FIGS. 15C-15D), and that additionally, pro-cell cycle genes were increased in PBMCs and pro-apoptosis genes were decreased.

FIG. 16 shows a computer system 1601 that is programmed or otherwise configured to, for example, perform methods of the disclosure.

FIG. 17 shows conserved and differential enrichment of immune cells and pathways in blood, lung, and airway of SARS-CoV2-infected patients.

FIGS. 18A-18C show elevated IFN expression in the lung tissue of COVID-19 patients. FIG. 18A: Normalized log 2 fold change RNA-seq expression values for IFN-associated genes from blood, lung, and airway of individual COVID-19 patients. The dotted line represents the expression of each gene in healthy individuals (for blood and lung) or PBMCs from COVID-19 patients (airway). FIG. 18B: Individual sample gene expression from the blood, lung, and airway was analyzed by GSVA for enrichment of IFN-related gene signatures. FIG. 18C: Normalized log 2 fold change RNA-seq expression values for anti-viral genes as in (section a). Generated using GraphPad Prism v8.4.2 (www.graphpad.com). #p<0.2, ##p<0.1, *p<0.05, **p<0.01, ***p<0.001, ****p<0.0001

FIGS. 19A-19D show that viral entry gene expression correlates with enhanced expression of inflammatory mediators in SARS-CoV2-infected lungs. FIGS. 19A-19B: Normalized log 2 fold change RNA-seq expression values for chemokines and chemokine receptors (section a) and IL-1 family members (FIG. 19B) from blood, lung, and airway of COVID-19 patients as in FIG. 18A. FIG. 19C: Individual sample gene expression from the blood, lung, and airway was analyzed by GSVA for enrichment of various lung tissue cell categories. FIG. 19D: Normalized log 2 fold change RNA-seq expression values for viral entry genes as in FIGS. 19A-19B. Generated using GraphPad Prism v8.4.2 (www.graphpad.com). #p<0.2, ##p<0.1, *p<0.05, **p<0.01, ***p<0.001, ****p<0.0001

FIGS. 20A-20F show that PPI analysis identifies different myeloid cell subsets and metabolic pathways in blood, lung, and airway of COVID-19 patients.

FIGS. 21A-21F show that different co-expression-derived myeloid populations are found in blood, lung, and airway of COVID-19 patients.

FIG. 22 shows an analysis of biological activities of myeloid subpopulations.

FIGS. 23A-23B show a pathway analysis of SARS-CoV-2 blood, lung, and airway.

FIG. 24 shows a graphical model of COVID-19 pathogenesis.

FIGS. 25A-25D show that metaclusters identify differentially expressed cell populations and functional gene clusters in SARS-CoV2 infected tissues and cell lines.

FIGS. 26A-26F shows an evaluation of macrophage gene signatures in myeloid-derived clusters from COVID-affected blood, lung and BAL fluid.

FIG. 27 shows heterogeneous expression of monocyte/myeloid cell genes in different CoV2 tissue compartments as compared to control.

FIG. 28 shows an analysis of biological activities of myeloid subpopulations.

FIGS. 29A-29E show a pathway analysis of SARS-CoV-2 lung tissue. FIG. 29A: Remaining significant upstream regulators operative in SARS-CoV-2 lung tissue predicted by IPA upstream regulator analysis. Upstream regulator analysis was also conducted on DEGs from each individual COVID-19 lung compared to healthy controls due to observed heterogeneity. FIG. 29B: significant results displayed for Lung1-CoV2 vs. Lung-CTL. FIG. 29C: significant results displayed for Lung2-CoV2 vs. Lung-CTL. Chemical reagents, chemical toxicants, and non-mammalian endogenous chemicals were culled from results. The boxes with the dotted outline separate small molecules/drugs/compounds that were predicted as upstream regulators from pathway molecules and complexes. FIGS. 29D-29E: IPA canonical signaling pathway analysis was conducted on individual COVID-19 lung samples. Pathways and upstream regulators were considered significant by |Activation Z-Score|≥2 and overlap p-value <0.01.

### DETAILED DESCRIPTION

Unless otherwise defined, all technical terms used herein have the same meaning as commonly understood by one of ordinary skill in the art to which this disclosure belongs.

### Certain Terms

As used herein, the singular forms “a,” “an,” and “the” include plural references unless the context clearly dictates otherwise. Any reference to “or” herein is intended to encompass “and/or” unless otherwise stated.

As used herein, the term “about” refers to an amount that is near the stated amount by 10%, 5%, or 1%, including increments therein.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, the singular forms “a,” “an,” and “the” include plural references unless the context clearly dictates otherwise. Any reference to “or” herein is intended to encompass “and/or” unless otherwise stated.

As used herein, the term “subject” refers to an entity or a medium that has testable or detectable genetic information. A subject can be a person, individual, or patient. A subject can be a vertebrate, such as, for example, a mammal. Non-limiting examples of mammals include humans, simians, farm animals, sport animals, rodents, and pets. The subject may be displaying a symptom(s) indicative of a health or physiological state or condition of the subject, such as a disease or disorder of the subject. As an alternative, the subject can be asymptomatic with respect to such health or physiological state or condition.

As used herein, the term “sample,” generally refers to a biological sample obtained from or derived from one or more subjects. Biological samples may be processed or fractionated before further analysis. Biological samples may include a whole blood (WB) sample, a PBMC sample, a tissue sample, a purified cell sample, or derivatives thereof. In some embodiments, a whole blood sample may be purified to obtain the purified cell sample. The term “derived from” used herein refers to an origin or source, and may include naturally occurring, recombinant, unpurified or purified molecules.

To obtain a blood sample, various techniques may be used, e.g., a syringe or other vacuum suction device. A blood sample can be optionally pre-treated or processed prior to use. A sample, such as a blood sample, may be analyzed under any of the methods and systems herein within 4 weeks, 2 weeks, 1 week, 6 days, 5 days, 4 days, 3 days, 2 days, 1 day, 12 hr, 6 hr, 3 hr, 2 hr, or 1 hr from the time the sample is obtained, or longer if frozen. When obtaining a sample from a subject (e.g., blood sample), the amount can vary depending upon subject size and the condition being screened. In some embodiments, at least 10 mL, 5 mL, 1 mL, 0.5 mL, 250, 200, 150, 100, 50, 40, 30, 20, 10, 9, 8, 7, 6, 5, 4, 3, 2, or 1 μL of a sample is obtained. In some embodiments, 1-50, 2-40, 3-30, or 4-20 μL of sample is obtained. In some embodiments, more than 5, 10, 15, 20, 25, 30, 35, 40, 45, 50, 55, 60, 65, 70, 75, 80, 85, 90, 95 or 100 μL of a sample is obtained.

As used herein the term “diagnose” or “diagnosis” of a status or outcome includes predicting or diagnosing the status or outcome, determining predisposition to a status or outcome, monitoring treatment of patient, diagnosing a therapeutic response of a patient, and prognosis of status or outcome, progression, and response to particular treatment.

The sample may be taken before and/or after treatment of a subject with a disease or disorder. Samples may be obtained from a subject during a treatment or a treatment regime. Multiple samples may be obtained from a subject to monitor the effects of the treatment over time. The sample may be taken from a subject known or suspected of having a disease or disorder for which a definitive positive or negative diagnosis is not available via clinical tests. The sample may be taken from a subject suspected of having a disease or disorder. The sample may be taken from a subject experiencing unexplained symptoms, such as fatigue, nausea, weight loss, aches and pains, weakness, or bleeding. The sample may be taken from a subject having explained symptoms. The sample may be taken from a subject at risk of developing a disease or disorder due to factors such as familial history, age, hypertension or pre-hypertension, diabetes or pre-diabetes, overweight or obesity, environmental exposure, lifestyle risk factors (e.g., smoking, alcohol consumption, or drug use), or presence of other risk factors.

In some embodiments, a sample can be taken at a first time point and assayed, and then another sample can be taken at a subsequent time point and assayed. Such methods can be used, for example, for longitudinal monitoring purposes to track the development or progression of a disease. In some embodiments, the progression of a disease can be tracked before treatment, after treatment, or during the course of treatment, to determine the treatment's effectiveness. For example, a method as described herein can be performed on a subject prior to, and after, treatment with a disease state or condition therapy to measure the disease's progression or regression in response to the disease state or condition therapy.

After obtaining a sample from the subject, the sample may be processed to generate datasets indicative of a disease or disorder of the subject. For example, a presence, absence, or quantitative assessment of nucleic acid molecules of the sample at a panel of disease state or condition-associated or interferon-associated genomic loci or may be indicative of a disease state or condition of the subject. Processing the sample obtained from the subject may comprise (i) subjecting the sample to conditions that are sufficient to isolate, enrich, or extract a plurality of nucleic acid molecules, and (ii) assaying the plurality of nucleic acid molecules to generate the dataset (e.g., microarray data, nucleic acid sequences, or quantitative polymerase chain reaction (qPCR) data). Methods of assaying may include any assay known in the art or described in the literature, for example, a microarray assay, a sequencing assay (e.g., DNA sequencing, RNA sequencing, or RNA-Seq), or a quantitative polymerase chain reaction (qPCR) assay.

In some embodiments, a plurality of nucleic acid molecules is extracted from the sample and subjected to sequencing to generate a plurality of sequencing reads. The nucleic acid molecules may comprise ribonucleic acid (RNA) or deoxyribonucleic acid (DNA). The extraction method may extract all RNA or DNA molecules from a sample. Alternatively, the extraction method may selectively extract a portion of RNA or DNA molecules from a sample. Extracted RNA molecules from a sample may be converted to cDNA molecules by reverse transcription (RT).

The sample may be processed without any nucleic acid extraction. For example, the disease or disorder may be identified or monitored in the subject by using probes configured to selectively enrich nucleic acid (e.g., RNA or DNA) molecules corresponding to a panel of disease state or condition-associated or interferon-associated genomic loci. The probes may be nucleic acid primers. The probes may have sequence complementarity with nucleic acid sequences from one or more of the panel of disease state or condition-associated or interferon-associated genomic loci. The panel of disease state or condition-associated or interferon-associated genomic loci may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, or more disease state or condition-associated or interferon-associated genomic loci.

The probes may be nucleic acid molecules (e.g., RNA or DNA) having sequence complementarity with nucleic acid sequences (e.g., RNA or DNA) of one or more genomic loci (e.g., disease state or condition-associated or interferon-associated genomic loci). These nucleic acid molecules may be primers or enrichment sequences. The assaying of the sample using probes that are selective for the one or more genomic loci (e.g., disease state or condition-associated or interferon-associated genomic loci) may comprise use of array hybridization, polymerase chain reaction (PCR), or nucleic acid sequencing (e.g., RNA sequencing or DNA sequencing, such as RNA-Seq).

The assay readouts may be quantified at one or more genomic loci (e.g., disease state or condition-associated or interferon-associated genomic loci) to generate the data indicative of the disease or disorder. For example, quantification of array hybridization or polymerase chain reaction (PCR) corresponding to a plurality of genomic loci (e.g., disease state or condition-associated or interferon-associated genomic loci) may generate data indicative of the disease or disorder. Assay readouts may comprise quantitative PCR (qPCR) values, digital PCR (dPCR) values, digital droplet PCR (ddPCR) values, fluorescence values, etc., or normalized values thereof.

Big Data Analysis Tools and Drug/Target Scoring Algorithms

The present disclosure provides systems and methods to perform data analysis using drug or target scoring algorithms and/or big data analysis tools. In various aspects, such drug or target scoring algorithms and/or big data analysis tools may be used to perform analysis of data sets including, for example, mRNA gene expression or transcriptome data, DNA genomic data, proteomic data, metabolomic data, other types of “-omic” data, or a combination thereof. Systems and methods of the present disclosure may use one or more of the following: a BIG-C™ big data analysis tool, an I-Scope™ big data analysis tool, a T-Scope™ big data analysis tool, a CellScan big data analysis tool, an MS (Molecular Signature) Scoring™ analysis tool, and a Gene Set Variation Analysis (GSVA) tool (e.g., P-Scope).

A method to assess a condition (e.g., COVID-19) of a subject may comprise using one or more data analysis tools and/or algorithms. The method may comprise receiving a dataset of a biological sample of a subject. Next, the method may comprise selecting one or more data analysis tools and/or algorithms. For example, the data analysis tools and/or algorithms may comprise a BIG-C™ big data analysis tool, an I-Scope™ big data analysis tool, a T-Scope™ big data analysis tool, a CellScan big data analysis tool, an MS (Molecular Signature) Scoring™ analysis tool, a Gene Set Variation Analysis (GSVA) tool (e.g., P-Scope), or a combination thereof. Next, the method may comprise processing the dataset using selected data analysis tools and/or algorithms to generate a data signature of the biological sample of the subject. Next, the method may comprise assessing the condition of the subject based on the data signature.

The BIG-C (Biologically Informed Gene Clustering) tool may be configured to sort large groups of genes into a set of functional groups (e.g., 53 functional groups). The functional groups are created utilizing publicly available information from online tools and databases including UniProtKB/Swiss-Prot, GO Terms, KEGG pathways, NCBI PubMed, and the Interactome. The functional groups may include one or more of: Active RNA, Anti-apoptosis, anti-proliferation, autophagy, chromatin remodeling, cytoplasm and biochemistry, cytoskeleton, DNA repair, endocytosis, endoplasmic reticulum, endosome and vesicles, fatty acid biosynthesis, cell surface, transcription, glycolysis and gluconeogenesis, golgi, immune cell surface, immune secreted, immune signaling, integrin pathway, interferon stimulated genes, intracellular signaling, lysosome, melanosome, MHC class I, MHC class II, microRNA processing, microRNA, mitochondrial transcription, mitochondria, mitochondria oxidative phosphorylation, mitochondrial TCA cycle, mRNA processing, mRNA splicing, non-coding RNA, nuclear receptor, nucleus and nucleolus, palmitoylation, pattern recognition receptors, peroxisomes, pro-apoptosis, pro-cell cycle, proteasome, pseudogenes, RAS superfamily, reactive oxygen species protection, secreted and extracellular matrix, transcription factors, transporters, transposon control, ubiquitylation and sumoylation, unfolded protein and stress, and unknown. Enrichment scores for each group are calculated based on an overlap p value to determine the functional groups over or under-expressed in the gene expression dataset. The BIG-C may be configured such that each gene is sorted into only one of the 53 functional groups, allowing for a quick and relatively simple understanding of types of genes enriched and co-expressed in a big dataset.

The I-Scope™ tool may be configured to identify immune infiltrates. Hematopoietic cells are unique in that they move throughout the body patrolling for threats to the host, and may infiltrate tissue sites not normally home to immune cells. I-Scope™ may be configured to identify hematopoietic cells through an iterative search of more than 17,000 genes identified in more than 50 microarray datasets. From this search, 1226 candidate genes are identified and researched for restriction in hematopoietic cells as determined by the HPA, GTEx and FANTOMS datasets (e.g., available at proteinatlas.org). 926 genes meet the criteria for being mainly restricted to hematopoietic lineages (brain, reproductive organ exclusions were permitted). These genes are researched for immune cell specific expression in 27 hematopoietic sub-categories: alpha beta T cell, T cell, regulatory T Cell, activated T cell, anergic T cell, gamma delta T cells, CD8 T, NK/NKT cell, NK cell, T & B cells, B cells, germinal center B cells, B cell and plasmacytoid dendritic cell, T &B & myeloid, B & myeloid, T & myeloid, MHC Class II expressing cell, monocyte, dendritic cell, plasmacytoid dendritic cells, myeloid cell, plasma cell, erythrocyte, neutrophil, low density granulocyte, granulocyte, and platelet. Transcripts are entered into I-Scope™ and the number of transcripts in each category determined. Odd's ratios are calculated with confidence intervals using the Fisher's exact test in R.

The T-Scope™ tool may be configured to help identify types of non-hematopoietic cells in gene expression datasets. T-Scope™ may be configured by downloading approximately 10,000 tissue enriched and 8,000 cell line enriched genes from the human protein atlas along with their tissue or cell line designation (e.g., available at proteinatlas.org). Genes found in more than four tissues are eliminated. Housekeeping genes described in the gene expression study by She et al. are also removed (e.g., as described by She et al., “Definition, conservation and epigenetics of housekeeping and tissue-enriched genes,” BMC Genomics 2009, 10:269, which is incorporated herein by reference in its entirety). This list is further curated by removing genes differentially expressed in 34 hematopoietic cell gene expression datasets and adding kidney specific genes from datasets downloaded from the GEO repository and processed by Ampel BioSolutions. The resulting categories of genes represent genes enriched in the following 42 tissue/cell specific categories: adrenal gland, breast, cartilage, cerebral cortex, uterine cervix, chondrocyte, colon, duodenum, endometrium, epididymis, esophagus fallopian tube, esophagus, fibroblast, heart muscle, keratinocyte, kidney, liver, lung, melanocyte, ovary pancreas, parathyroid gland, placenta, podocyte, prostrate, rectum, salivary gland, seminal vesicle, skeletal muscle, skin, small intestine, smooth muscle, stomach, synoviocyte, testis, kidney loop of henle, kidney proximal tubule, kidney distal tubule, and kidney collecting duct.

The CellScan tool may be a combination of I-Scope™ and T-Scope™, and may be configured to analyze tissues with suspected immune infiltrations that should also have tissue specific genes. CellScan may potentially be more stringent than either I-Scope™ or T-Scope™ because it may be used to distinguish resident tissue cells from non-resident hematopoietic cells.

The MS (Molecular Signature) Scoring tool may be configured to assess specific pathways in a disease state. Information on genes that encode for proteins that participate in a specific signaling pathway, and whether the gene product promotes or inhibits the pathway, are compiled and curated through literature mining. Curated pathways presented by the company include CD40-CD40ligand, IL-6, IL-12/23, TNF, IL-17, IL-21, S1P1, IL-13 and PDE4, but this method may be used for any known signaling pathway with available data. To determine if a signaling pathway is over or under-expressed in a microarray dataset, the gene list for each signaling pathway may be queried against the limma differentially expressed genes from a disease state compared to healthy controls, and the differentially expressed genes in the signaling pathway may be identified for each set. The fold changes for genes that promoted the pathway may be added together and the fold changes for genes that inhibited the pathway may be subtracted from the score. This total score may be normalized based on the number of genes that could be detected on the specific microarray platform used for the experiment. Activation scores of −100 to +100 may be determined using this method with negative scores indicating an inhibition of the specific pathway in the disease state and positive scores indicating an up-regulation of a specific pathway in the disease state. The Fischer's exact test may be performed to determine if there was sufficient overlap of genes between the experimental differentially expressed genes and the genes in the signaling pathway.

Gene Set Variation Analysis (GSVA) may be performed (for example, as described in Catalina et al. (2019, Communications Biology, “Gene expression analysis delineates the potential roles of multiple interferons in systemic lupus erythematosus”, which is incorporated herein by reference in its entirety) to determine enrichment of signaling pathways in individual patient samples. Gene set variation analysis may be performed using an open source software package for the coding language R available at the R Bioconductor (bioconductor.org), e.g., as described by Hanzelman et al., (“GSVA: gene set variation analysis for microarray and RNA-Seq data,” BMC Bioinformatics, 2013, which is incorporated herein by reference in its entirety). The modules of genes to interrogate the datasets may be developed. Modules of genes determined to represent a specific signaling pathway or process may be identified (e.g., using publicly available datasets). For example, the IFNB1 signaling pathway is taken from a publicly available gene expression dataset of peripheral blood cells treated with IFNB1 in vitro. Genes co-expressed in this dataset (genes either all increased or decreased compared to control treated peripheral blood) are used to create modules of genes representing the IFNB1 signaling pathway, and GSVA is used to determine the enrichment of this set of genes and hence the IFNB1 signaling pathway in individual patient and control samples.

BIG-C™ Big Data Analysis Tool

BIG-C® is a fast and efficient cloud-based tool to functionally categorize gene products. With coverage of over 80% of the genome, BIG-C® leverages publicly available databases such as UniProtKB/Swiss-Prot, GO terms, KEGG pathways, NCBI PubMed and Interactome to place genes into 53 functional categories. The sorting into only one of 53 functional groups allows for a quick and relatively simple understanding of types of genes enriched and co-expressed in a big dataset. This assists in deriving further insights from genes expressed for a given disease state in human or pre-clinical mouse models.

BIG-C® can be used to functionally categorize immunological genes that are not covered in cancer databases such as GO and KEGG (e.g., as described by Grammer et al. 2016, “Drug repositioning in SLE: crowd-sourcing, literature-mining and Big Data analysis,” Lupus, 25 (10), 1150-1170, which is incorporated herein by reference in its entirety). Using a knowledge base of over 5000 patients with systemic lupus erythematosus (SLE), over 16432 genes may be each placed into one of 53 BIG-C® functional categories, and statistical analysis may be performed to identify enriched categories. BIG-C® categories may be cross-examined with the GO and KEGG terms to obtain additional information and insights.

A sample BIG-C® workflow may comprise the following steps. First, SLE genomic datasets are derived from whole blood, peripheral blood mononuclear cells, affected tissues, and purified immune cells. Second, datasets are analyzed using DE analysis or Weighted Gene Coexpression Network Analysis (WGCNA). Third, expressed genes are annotated using publicly available databases (e.g., UniProtKB/Swiss-Prot database, Human Immunodeficiencies database, Mouse MGI database, Entrez Molecular Sequence database, PubMed, and the Human Tissue Atlas). Fourth, signatures are cross-referenced with purified single-cell microarray datasets and RNAseq experiments. Fifth, BIG-C® is leveraged to separate the individual annotated genes into one of 53 functional categories shown in Table 1 (e.g., as described by Labonte et al. 2018, “Identification of alterations in macrophage activation associated with disease activity in systemic lupus erythematosus,” PloS one, 13 (12), e0208132, which is incorporated herein by reference in its entirety). Sixth, chi-squared analysis is used to determine enriched categories of interest from overlap p-values. Seventh, enriched categories are cross-examined with GO and KEGG terms to derive key insights for further analysis.

TABLE 1
BIG-C Categories

| Immune | | | | | | | | |
|---|---|---|---|---|---|---|---|---|
| Cell | General Cell | Immune | Intracellular | MHC Class | MHC Class | Secreted | | Pat. Recog. |
| Surface | Surface | Signaling | Signaling | I | II | Immune | Secreted ECM | Receptors |
| Interferon | PRO-Cell | Anti-Cell | PRO | Anti | Unfold Prot. | Proteasome | Autophagy | Ubiquity lation |
| Gene Sig | Cycle | Cycle | Apoptosis | Apoptosis | Stress | | | |
| General | Transcript. | Nuc | Chromatin | DNA | mRNA | mRNA | MicroRNA | Cytoskeleton |
| Transcript. | Factors | Horm. | Remodel | Repair | Translation | Splicing | Processing | |
| | | Receptors | | | | | | |
| Integrin | RAS | WNT | Lysosome | Endocytosis | Endosome | Endoplas. | Oxidative | TCA Cycle |
| Pathway | Superfamily | Signaling | | | & Vesicles | Retic. | Phosphor. | |
| Mito. DNA | Mito | FA | Transporters | Cytoplasm | Peroxisomes | ROS | Nuclear & | Active RNA |
| toRNA | | Biosynth | | Biochem | | Protection | Nucleolus | |
| MicroRNA | Melanosome | Unknown | Pseudogenes | Transposon | Golgi | Glycolysis | Palmitoylation | |
| | | | | Control | | | | |

I-Scope™ Big Data Analysis Tool

I-Scope™ may be a tool configured for cross-examining the presence and activity of varying types of immune cell infiltrates with observed gene expression patterns. It may take annotated gene expression data and analyze it for hematopoietic cell lineage. I-Scope™ can be used downstream of the BIG-C® (Biologically Informed Gene-Clustering) tool in that it helps to provide even more insight into the nature of the genes being expressed after categorization.

I-Scope™ addresses the need to understand the involvement of specific cells for a given disease state. While it is helpful to understand the relative up-regulation and down-regulation at the gene expression level, it is even more informative to understand specifically in which cells this is occurring. I-Scope™ may be configured to identify hematopoietic cells through an iterative search of more than 17,000 genes identified in more than 50 microarray datasets (e.g., as described by Hubbard et al., “Analysis of Lupus Synovitis Gene Expression Reveals Dysregulation of Pathogenic Pathways Activated within Infiltrating Immune Cells,” Arthritis Rheumatol, 2018; 70 (suppl 10), which is incorporated herein by reference in its entirety). I-Scope™ may function by restricting the analysis to genes of hematopoietic cell heritage and allow for cross-checking against purified single-cell experiments or datasets. The cross-check confirms and categorizes specific transcript signatures to the 28 hematopoietic cell sub-categories shown in Table 2, ultimately allowing for cellular activity analysis across multiple samples and disease states. When combined with BIG-C® categories, the cellular activity can be correlated to specific functions within a given cell type.

TABLE 2
I-Scope™ Cell Sub-Categories

| Monos/ | Plasma | | | | | |
|---|---|---|---|---|---|---|
| Macs | Cells | T-Cells | B-Cells | Dendritic | T&B Cells | CD8 T |
| Myeloid | Tact | LDG | Hematopoietic | Neutrophil | Ag | Granulocytes |
| Cells | | | | | Presentation | |
| Platelets | pDC | T, B, Mono | Langerhans | Bact | Mono and | Erythrocytes |
| | | | | | B | |
| Mast Cell | T reg | Gd T | T anergic | FDC | CD4T | T/NK/NKT |
| | | | | | | Cells |

A sample I-Scope™ workflow may comprise the following steps. First, candidate genes are identified from datasets (associated with a disease state or condition) potentially associated with immune cell expression. Second, using HPA, GTEx, and FANTOMS datasets, expression signatures associated with hematopoietic cell lineage are identified. Third, signatures are cross-referenced with purified single-cell microarray datasets and RNAseq experiments. Fourth, transcripts are categorized into 28 hematopoietic cell sub-categories and assess cellular expression across different samples and disease states. Odd's ratios are calculated with confidence intervals using the Fisher's exact test in R. An I-Scope™ signature analysis for a given sample may lead to the I-Scope™ signature analysis across multiple samples and disease states.

T-Scope™ Big Data Analysis Tool

The T-Scope™ tool may be configured for cross-examining gene expression signatures of a given sample with a database of non-hematopoietic cell types (e.g., as described by Hubbard et al., “Analysis of Gene Expression from Systemic Lupus Erythematosus Synovium Reveals Unique Pathogenic Mechanisms [Abstract], Annual Meeting of the American College of Rheumatology; June 2019; Chicago, Ill., which is incorporated herein by reference in its entirety). T-Scope™ may comprise a database of 704 transcripts allocated to 45 independent categories. Transcripts detected in the sample are matched to one of the cellular categories within the T-Scope™ tool to derive further insights on tissue cell activity. T-Scope™ can be used downstream of the BIG-C® (Biologically Informed Gene-Clustering) tool to understand which tissue cell types are present. In conjunction with I-Scope™ (which provides information related to immune cells), T-Scope™ can be performed to provide a complete view of all possible cell activity in a given sample.

T-Scope™ addresses the need to understand the involvement of specific tissue cells for a given disease state. While it is helpful to understand the relative up-regulation and down-regulation at the gene expression level, it is even more informative to understand specifically in which cells this is occurring. T-Scope™ may be configured by downloading a set of approximately 10,000 tissue enriched and 8,000 cell line enriched genes from the Human Protein Atlas along with their tissue or cell line designation. Genes differentially expressed in hematopoietic cell datasets are removed and kidney specific genes are added from the GEO repository. T-Scope™ may function by restricting the analysis to genes of known tissue cell heritage and allow for cross-checking against purified single-cell experiments or datasets. The cross-check confirms and categorizes specific transcript signatures to the 45 tissue cell sub-categories (as shown in Table 3), ultimately allowing for cellular activity analysis across multiple samples and disease states. When combined with BIG-C® categories, the cellular activity can be correlated to specific functions within a given tissue cell type.

TABLE 3
T-Scope™ 45 Categories of Tissue Cells

| Adipose | Adrenal | | | Cerebral | Cervix, | | | |
|---|---|---|---|---|---|---|---|---|
| Tissue | Gland | Breast | Cartilage | Cortex | Uterine | Chondrocyte | Colon | Dendritic |
| Duodenum | Endometrium | Endothelial | Epididymis | Erythrocytes | Esophagus | Fallopian | Fibroblast | Gallbaldder |
| | | | | | | Tube | | |
| Heart Muscle | Keratinocyte | Keratinocyte | Kidney | Kidney | Kidney Loop | Kidney | Kidney | Kidney |
| | | Skin | | Distal | | Proximal | Tubule Duct | Tubule |
| | | | | Tubules | | Tubules | | |
| Langherhans | Liver | Lung | Melanocyte | Podocyte | Prostate | Rectum | Salivary | Seminal |
| | | | | | | | Gland | Vesicle |
| Skeletal | Skin | Small | Smooth | Stomach | Synoviocyte | Testis | Thyroid | Urinary |
| Muscle | | Intenstine | Muscle | | | | Gland | Bladder |

A sample T-Scope™ workflow may comprise the following steps. First, candidate genes are identified from differential expression datasets (associated with a disease state or condition) potentially associated with tissue cell expression. Second, using publicly available databases, expression signatures associated with potential tissue cell activity are identified. Third, signatures are cross-referenced with microarray, scRNAseq or RNAseq experiments. Fourth, transcripts are categorized into 45 tissue cell sub-categories and cellular expression is assessed across different samples and disease states. Results may be obtained using T-Scope™ in combination with I-Scope™ for identification of cells post-DE-analysis.

Cell Scan Big Data Analysis Tool

A cloud-based genomic platform may be configured to provide users with access to CellScan™, which comprises a suite of tools for the identification, analysis, and prioritization of targets for drug development and/or repositioning. This platform is powered by a database containing the genomic information gathered from 5000+ autoimmune patients. The cloud-based genomic platform may leverage results from RNAseq and microarray experiments in conjunction with clinical information, such as medication and lab tests, to provide previously undiscovered insights.

CellScan™ may go beyond typical ‘omics analysis by performing one or more of the following: functionally categorizing genes and their products (e.g., using BIG-C®); deconvolving gene expression data to identify unique immunological cell types from blood or biopsy samples (e.g., using I-Scope™); identifying tissue specific cell from biopsy samples (e.g., using T-Scope™); identifying receptor-ligand interactions and subsequent signaling pathways (e.g., using MS-Scoring™); ranking genes and their products for targeting by drugs and miRNA mimetics; and prioritizing FDA-approved drugs and drugs-in-development for treatment in patients or pre-clinical models.

CellScan™ applications may include one or more of: Biomarker Discovery, Disease Mechanisms, Drug Mechanism of Action, Drug Mechanism of Toxicity, and Target Identification and Validation. Experimental approaches supported by CellScan™ may include one or more of: lncRNA, Metabolomics, MicroArray, miRNA, mRNA, qPCR, Proteomics, and RNAseq.

Data analysis and interpretation with CellScan™ may build on comprehensive, manually curated content of a knowledge base. Powerful, quick, and efficient tools may be used to perform deep analysis of NGS and miRNA data to identify gene function, immunological and tissue cell type, pathways, and target/drug appropriate for a specific disease state.

CellScan™ features may be configured to optimize or maximize the impact of information that surfaces in an analysis so that interpretation of a dataset is comprehensive and elucidates actionable insights. These features may include one or more of: NGS RNAseq data analysis, biomarker scoring, and prioritizing targets and drugs for human clinical trials and/or pre-clinical models. The NGS RNAseq data analysis may comprise interrogating RNA and miRNA data for function, cell-type (immunological or tissue) and pathways. The biomarker scoring may comprise using a knowledge base and gene expression data to assess and prioritize biomarkers associated with a target disease or phenotype. The target/drug prioritization may comprise leveraging objective scoring of targets and drugs based on parameters such as scientific rationale, evidence in mouse/human cells, prior clinical data, overall drug properties, and the risk of adverse events.

The knowledge base may be a repository created from millions of individual pieces of information gathered about genes, cells, tissues, drugs, and diseases, and manually reviewed for accuracy and includes rich contextual details and links to original publications. The knowledge base may enable access to relevant and substantiated knowledge from primary literature as well as public and private databases for comprehensive interpretation of NGS/RNAseq data elucidating function/pathways and prioritize targets/drugs for given disease states. Table 4 shows an example list of reference databases for the content in CellScan™, with both human and mouse species-specific identifiers supported.

TABLE 4
Reference Databases for Content in CellScan ™

| | | | |
|---|---|---|---|
| Affymetrix | Entrez Gene | HPA | scRNAseq |
| Agilent | FANTOM5 | Illumina | STITCH |
| BrainArray | GenBank | Interactome | Mouse |
| | | | Genome |
| | | | Database |
| | | | (MGD) |
| CAS Registry | Gene Symbol-human | KEGG | UCSC |
| Number | (Hugo/HGNC) | | (hg 18) |
| Clinicaltrials.gov | Gene Symbol-mouse | LINCS/CLUE | UCSC |
| | (Entrez Gene) | | (hg 19) |
| CodeLink | GNF Tissue | Mosby's Drug | Unigene |
| | Expression Body | Consult | |
| | Atlas | | |
| DrugBank | GO terms | NCBI PubMed | Uniprot/ |
| | | | Swiss-Prot |
| | | | Accession |
| Drugs@FDA | Goodman & Gilman's | NCI-60 Cell Line | |
| | Pharmacological Basis | Expression Atlas | |
| | of Therapeutics | | |
| Ensembl | GTEx | Refseq | |

MS (Molecular Signature) Scoring™ Analysis Tool

MS-Scoring™ may be configured to identify receptor-ligand interactions and predict ongoing signaling pathways. In addition, MS-Scoring™ may be used to validate molecular pathways as potential targets for new or repurposed drug therapies. The specificity of next-generation drug therapies requires a way to understand the potential of a given therapy to act on the intended biochemical target. Moreover, a potential application of this is the repositioning of drug therapies that may have the correct biochemical targeting to address multiple clinical needs beyond the initial intended therapeutic value.

MS-Scoring™ may be specifically developed to address gaps in the QIAGEN IPA® (Ingenuity Pathway Analysis) tool that does not contain many immunologically relevant pathways. Similar to IPA®, MS-Scoring™ 1 may use log-fold change information to score the target and its signaling pathway to verify the viability of the targets. If the fold-change of the genes of a signaling pathway appears to be upregulated or inhibitors appear to be downregulated, MS-Scoring™ 1 may provide a score of +1. Conversely if the genes of a signaling pathway appear downregulated or the inhibitors upregulated, MS-Scoring™ 1 may provide a score of −1. A score of zero may be provided if no fold-change is observed. The scores may then be summed and normalized across the entire pathway to yield a final % score between −100 (inhibition) and +100 (up-regulation). Higher absolute magnitude scores, scores that are close to −100 or +100, may indicate a high potential for therapeutic targeting. The Fischer's exact test may be performed to determine if there is sufficient overlap of genes between the experimental differentially expressed genes and the genes in the signaling pathway.

A sample MS-Scoring™ 1 workflow may comprise the following steps. First, potential drugs and pathways are identified by LINCS (Library of Integrated Network-Based Cellular Signatures) as candidates for therapeutic intervention. Second, MS-Scoring™ 1 is used to evaluate individual transcript elements of the target pathway. Third, signatures are cross-referenced with purified single-cell microarray datasets and RNAseq experiments. Fourth, scores are compiled and normalized to provide an overall % score for the pathway and higher absolute magnitude scores indicate a higher potential for therapeutic targeting.

MS-Scoring™ 2 may utilize custom-defined gene modules that represent a signaling pathway or process and is particularly useful for gene expression datasets from microarray or RNAseq. The MS-Scoring™ 2 tool may be configured to take a deeper look at signaling pathways analyzed using the MS-Scoring™ 1. The tool may analyze raw gene expression data and assess enrichment by the Gene Set Variation Analysis (as described herein), which assigns an indexed score to the individual co-expressed pathways between −1 and +1 indicating levels of down-regulation and up-regulation respectively.

A sample MS-Scoring™ 2 workflow may comprise the following steps. First, a signaling pathway of interest is selected from the MS-Scoring™ 2 menu. Second, a raw gene expression data is inputted into the MS-Scoring™ 2 tool. Third, enrichment of signaling pathway (s) is assessed on a patient by patient basis. Fourth, the data can then be used to drive insight for the target signaling pathways in individual patient samples.

Gene Set Variation Analysis (GSVA)

Gene Set Variation Analysis (GSVA) algorithms may be performed (for example, as described in Catalina et al. (2019, Communications Biology, “Gene expression analysis delineates the potential roles of multiple interferons in systemic lupus erythematosus”, which is incorporated herein by reference in its entirety) to determine enrichment of signaling pathways in individual patient samples. Gene set variation analysis may be performed using an open source software package for the coding language R available at the R Bioconductor (bioconductor.org), e.g., as described by Hanzelman et al., (“GSVA: gene set variation analysis for microarray and RNA-Seq data,” BMC Bioinformatics, 2013, which is incorporated herein by reference in its entirety) and as described by [R Core Team (2020). R: A language and environment for statistical computing. R Foundation for Statistical Computing, Vienna, Austria. www.R-project.org/], which is incorporated herein by reference in its entirety. The modules of genes to interrogate the datasets may be developed. Modules of genes determined to represent a specific signaling pathway or process may be identified (e.g., using publicly available datasets). For example, the IFNB1 signaling pathway is taken from a publicly available gene expression dataset of peripheral blood cells treated with IFNB1 in vitro. Genes co-expressed in this dataset (genes either all increased or decreased compared to control treated peripheral blood) are used to create modules of genes representing the IFNB1 signaling pathway, and GSVA is used to determine the enrichment of this set of genes and hence the IFNB1 signaling pathway in individual patient and control samples.

A GSVA-based data analysis tool may be developed for use in analyzing specific sets of gene pathways. The GSVA-based data analysis tool (e.g., P-Scope) may use a GSVA statistical test-based tool using different sets of genes to analyze certain pathways. Such sets of genes may include, for example, human genes, mouse genes, or a combination thereof.

Computer Systems

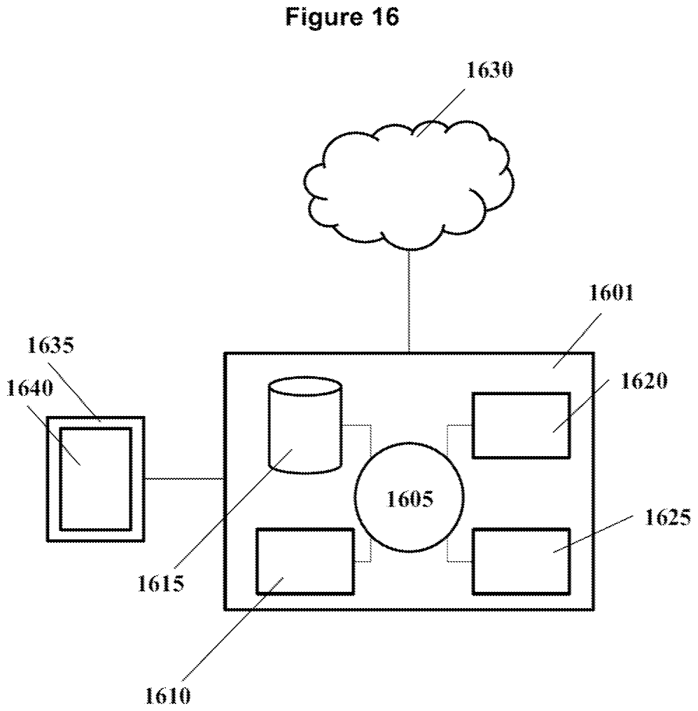

The present disclosure provides computer systems that are programmed to implement methods of the disclosure. FIG. 16 shows a computer system 1601 that is programmed or otherwise configured to, for example, perform methods of the disclosure.

The computer system 1601 can regulate various aspects of analysis, calculation, and generation of the present disclosure, such as, for example, performing methods of the disclosure. The computer system 1601 can be an electronic device of a user or a computer system that is remotely located with respect to the electronic device. The electronic device can be a mobile electronic device.

The computer system 1601 includes a central processing unit (CPU, also “processor” and “computer processor” herein) 1605, which can be a single core or multi core processor, or a plurality of processors for parallel processing. The computer system 1601 also includes memory or memory location 1610 (e.g., random-access memory, read-only memory, flash memory), electronic storage unit 1615 (e.g., hard disk), communication interface 1620 (e.g., network adapter) for communicating with one or more other systems, and peripheral devices 1625, such as cache, other memory, data storage and/or electronic display adapters. The memory 1610, storage unit 1615, interface 1620 and peripheral devices 1625 are in communication with the CPU 1605 through a communication bus (solid lines), such as a motherboard. The storage unit 1615 can be a data storage unit (or data repository) for storing data. The computer system 1601 can be operatively coupled to a computer network (“network”) 1630 with the aid of the communication interface 1620. The network 1630 can be the Internet, an internet and/or extranet, or an intranet and/or extranet that is in communication with the Internet.

The network 1630 in some cases is a telecommunication and/or data network. The network 1630 can include one or more computer servers, which can enable distributed computing, such as cloud computing. For example, one or more computer servers may enable cloud computing over the network 1630 (“the cloud”) to perform various aspects of analysis, calculation, and generation of the present disclosure, such as, for example, performing methods of the disclosure. Such cloud computing may be provided by cloud computing platforms such as, for example, Amazon Web Services (AWS), Microsoft Azure, Google Cloud Platform, and IBM cloud. The network 1630, in some cases with the aid of the computer system 1601, can implement a peer-to-peer network, which may enable devices coupled to the computer system 1601 to behave as a client or a server.

The CPU 1605 may comprise one or more computer processors and/or one or more graphics processing units (GPUs). The CPU 1605 can execute a sequence of machine-readable instructions, which can be embodied in a program or software. The instructions may be stored in a memory location, such as the memory 1610. The instructions can be directed to the CPU 1605, which can subsequently program or otherwise configure the CPU 1605 to implement methods of the present disclosure. Examples of operations performed by the CPU 1605 can include fetch, decode, execute, and writeback.

The CPU 1605 can be part of a circuit, such as an integrated circuit. One or more other components of the system 1601 can be included in the circuit. In some cases, the circuit is an application specific integrated circuit (ASIC).

The storage unit 1615 can store files, such as drivers, libraries and saved programs. The storage unit 1615 can store user data, e.g., user preferences and user programs. The computer system 1601 in some cases can include one or more additional data storage units that are external to the computer system 1601, such as located on a remote server that is in communication with the computer system 1601 through an intranet or the Internet.

The computer system 1601 can communicate with one or more remote computer systems through the network 1630. For instance, the computer system 1601 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1601 via the network 1630.

Methods as described herein can be implemented by way of machine (e.g., computer processor) executable code stored on an electronic storage location of the computer system 1601, such as, for example, on the memory 1610 or electronic storage unit 1615. The machine executable or machine-readable code can be provided in the form of software. During use, the code can be executed by the processor 1605. In some cases, the code can be retrieved from the storage unit 1615 and stored on the memory 1610 for ready access by the processor 1605. In some situations, the electronic storage unit 1615 can be precluded, and machine-executable instructions are stored on memory 1610.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 1601, can be embodied in programming. Various aspects of the technology may be thought of as “products” or “articles of manufacture” typically in the form of machine (or processor) executable code and/or associated data that is carried on or embodied in a type of machine-readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine-readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer-readable media may be involved in carrying one or more sequences of one or more instructions to a processor for execution.

The computer system 1601 can include or be in communication with an electronic display 1635 that comprises a user interface (UI) 1640 for providing, for example, a visual display. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit 1605. The algorithm can, for example, perform methods of the disclosure.

### EXAMPLES

The following illustrative examples are representative of embodiments of the software applications, systems, and methods described herein and are not meant to be limiting in any way.

### Example 1: Comprehensive Gene Expression Analysis Reveals Targetable Pathologic Processes in Blood, Lung, and Airway of COVID-19 Patients

Abstract

SARS-CoV2 is a previously uncharacterized coronavirus and causative agent of the COVID-19 pandemic. COVID-19 typically causes mild respiratory symptoms, but may escalate to acute respiratory distress syndrome (ARDs) with an increased risk of respiratory failure and death. However, the trajectory of disease progression and the status of affected tissues in COVID-19 patients has not been elucidated. We performed a comprehensive analysis of gene expression data from the blood, lung, and airway of COVID-19 patients to better understand the host response to SARS-CoV2 infection. Our results indicate that COVID-19 pathogenesis is driven by populations of myeloid-lineage cells with highly inflammatory but distinct transcriptional signatures, suggesting a progression in activation from the periphery to the lung tissue. In addition, through analysis of immune cells and inflammatory pathways enriched in each compartment, we have built a model of the systemic response to SARS-CoV2 and identified therapeutics targeting key upstream regulators of pathways contributing to COVID-19 pathogenesis.

Introduction

Coronaviruses (CoV) are a group of enveloped single positive stranded RNA viruses named for the spike proteins on their surface that resemble a crown (Fung and Liu, 2019). Seven coronaviruses have now been found to infect humans, causing mild to severe respiratory and intestinal illnesses including an estimated 15% of common colds (Cui et al., 2019; Greenberg, 2016). In the past two decades, three global pandemics have originated from coronaviruses capable of infecting the lower respiratory tract resulting in heightened pathogenicity and high mortality rates in humans. In 2002-2003, severe acute respiratory syndrome coronavirus (SARS-CoV) lead to greater than 8,000 cases with a mortality rate of nearly 10% (Drosten et al., 2003; Fung and Liu, 2019). Subsequently, Middle East respiratory syndrome coronavirus (MERS-CoV) emerged in 2012, leading to over 2,000 confirmed cases and a mortality rate of approximately 35% (Chafekar and Fielding, 2018; Fung and Liu, 2019). We are currently in the midst of a pandemic stemming from a new coronavirus strain, severe acute respiratory syndrome coronavirus 2 (SARS-CoV2), the causative agent of coronavirus disease 2019 (COVID-19). In the majority of cases, patients exhibit mild symptoms, whereas in more severe cases, patients may develop acute respiratory distress syndrome (ARDS), which leads to lung injury and death from respiratory failure (Chen et al., 2020a; Zhang et al., 2020).

SARS-CoV2 utilizes the SARS-CoV receptor, ACE2, in conjunction with the spike protein activator, TMPRSS2, to infect host cells (Hoffmann et al., 2020). Expression of ACE2 and TMPRSS2 has been detected in multiple tissues including lung epithelium and vascular endothelium, (Lovren et al., 2008; Sungnak et al., 2020) which are likely to be the first cells infected by the virus. However, at this time, there is still incomplete information available regarding the host response to SARS-CoV2 infection and the perturbations resulting in a severe outcome. Despite this, clues can be derived from our knowledge of the immune response to infection by other respiratory viruses, including SARS-CoV and MERS-CoV. After infection, viruses are typically detected by pattern recognition receptors (PRRs) such as the inflammasome sensor NLRP3, which signal the release of interferons and inflammatory cytokines including the IL-1 family, IL-6, and TNF which activate a local and systemic response to infection (Kelley et al., 2019; Lazear et al., 2019). This involves the recruitment, activation, and differentiation of innate and adaptive immune cells including neutrophils, inflammatory myeloid cells, CD8 T cells, and natural killer (NK) cells (Newton et al., 2016). Resolution of infection is largely dependent on the cytotoxic activity of CD8 T cells and NK cells, which enables clearance of virus-infected cells and thus acts to prevent further spread of the virus (Newton et al., 2016). Failure to clear virus-infected cells may facilitate a hyper-inflammatory state termed “cytokine storm”, macrophage activation syndrome (MAS), or haemophagocytic lymphohystocytosis (HLH) and ultimately damage to the infected lung (Crayne et al., 2019; McGonagle et al., 2020). Clearance of virus also coincides with development of neutralizing antibodies, although recent studies have suggested that 30% of subjects who clear SARS-CoV2 do not develop neutralizing antibodies (Tay et al.). Moreover, the role of the anti-SARS-CoV2 antibody is complex, in that in non-human primates, anti-viral antibody may exacerbate the lung pathology, a phenomenon also reported in older subjects infected with the virus (Wang et al., 2020).

The novelty of SARS-CoV2 and thus the lack of comprehensive knowledge regarding the progression of COVID-19 disease, has constrained our ability to develop effective treatments for infected patients. One means to obtain a comprehensive knowledge of the host response to SARS-CoV2 is to examine gene expression in relevant tissues. A limited number of studies have been reported and have suggested that the disease generally induces a dysfunctional immune response involving lymphopoenia, increased inflammatory cell recruitment to the lung, and an increase in local and systemic release of pro-inflammatory cytokines or “cytokine storm”, conditions which are exacerbated in more severe cases (Tay et al.). However, because of the small number of samples and limited analysis, a full picture of the biological state of SARS-CoV2-affected tissues has not emerged. To address this, we have assessed all of the available SARS-CoV2 gene expression datasets using a number of orthogonal bioinformatic tools to better understand the host response to SARS-CoV2 infection. As a result, we have identified immune cell types and inflammatory pathways critical for COVID-19 pathogenesis. Furthermore, we have characterized differences in transcriptional signatures between these compartments to develop a model for the systemic response to SARS-CoV2 infection and identified potential drugs to target harmful inflammatory mediators in COVID-19 patients.

Results

Gene Expression Analysis of Blood, Lung, and Airway of COVID-19 Patients

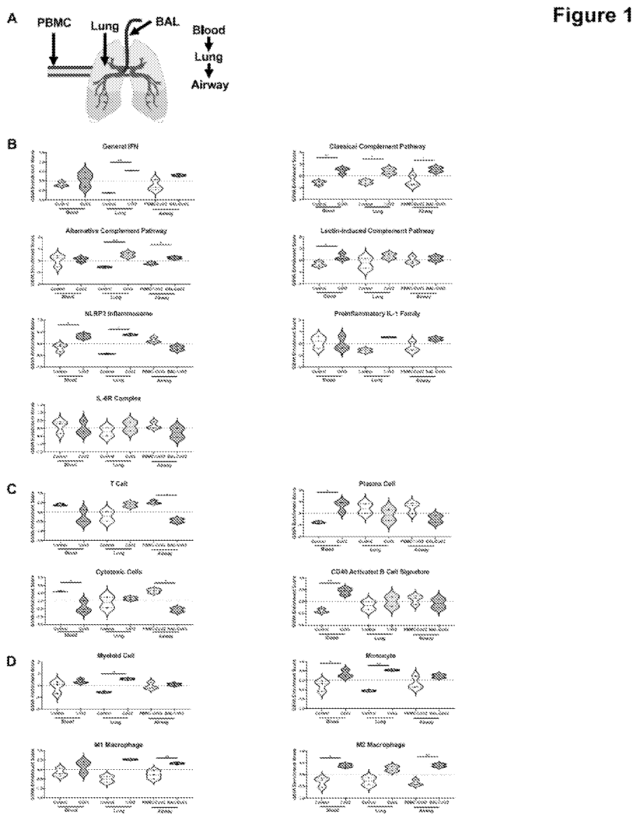

Gene expression reflects the current state of cellular activity of cells or organs and can be employed to delineate changes in cellular composition and/or function in a sample. A limited number of gene expression profiles are available from patients with COVID-19 and have yielded some insights into the pathogenic processes triggered by infection with SARS-CoV-2 (Blanco-Melo et al., 2020; Wei et al., 2020; Xiong et al., 2020). We reasoned that a more comprehensive analysis of all available gene sets from blood, lung and airway using a number of complementary orthogonal approaches might provide a more complete view of the nature of the COVID-19 inflammatory response and the potential points of intervention that might have therapeutic value. To characterize the pathologic response to SARS-CoV2 infection manifested in the peripheral blood and lung of COVID-19 patients, we analyzed RNA sequencing (RNA-seq) data from peripheral blood mononuclear cells (PBMCs) and postmortem lung tissue of COVID-19 patients and healthy controls as well as bronchoalveolar lavage (BAL) fluid of COVID-19 patients (Blanco-Melo et al., 2020; Xiong et al., 2020). We first determined changes in gene expression in the blood (PBMC-CTL vs PBMC-CoV2) and lung (Lung-CTL vs Lung-CoV2). Because no control BAL fluids were associated with the BAL-CoV2 samples, we compared BAL-CoV2 to PBMC-CoV2 from the same dataset to avoid effects related to batch and methodology. These results must be viewed with caution, understanding that the comparison can only show changes in gene expression profiles between different compartments. Despite that, important differences were observed. Overall, we found 4,277 differentially expressed genes (DEGs) in the blood (2,175 up and 2,102 down), 2,261 DE genes in the lung (711 up and 1550 down), and 9,183 DE genes in the airway (4,263 up and 4,920 down). From these comparisons, we traced transcriptional changes in the progression of the pathologic response to SARS-CoV2 through these three compartments from activation and mobilization of immune cells in the blood, to infiltration into the lung tissue and airway of infected patients (FIG. 1A).

Conserved and Differential Enrichment of Immune Cells and Pathways in Blood, Lung, and Airway of COVID-19 Patients

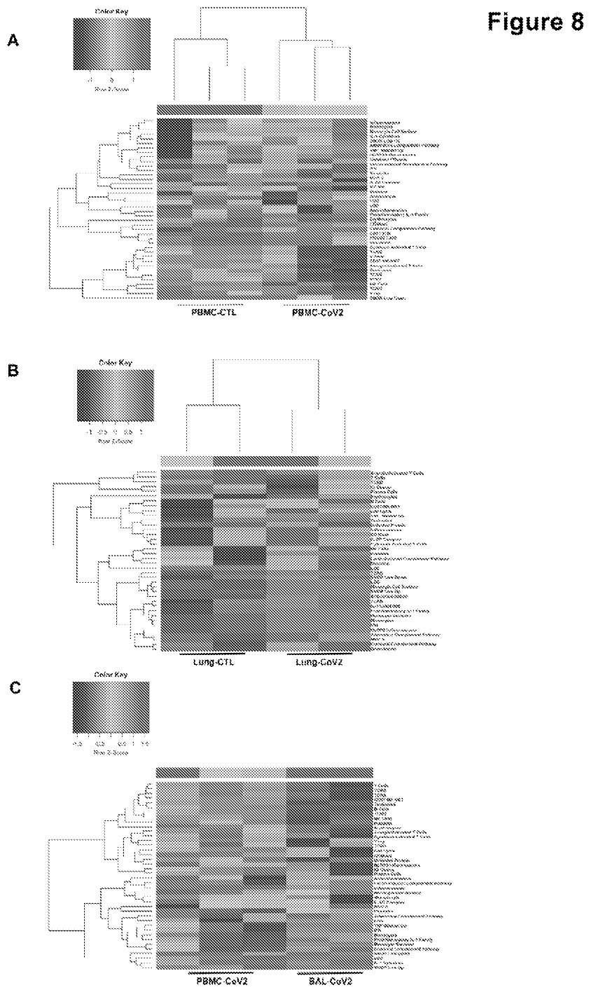

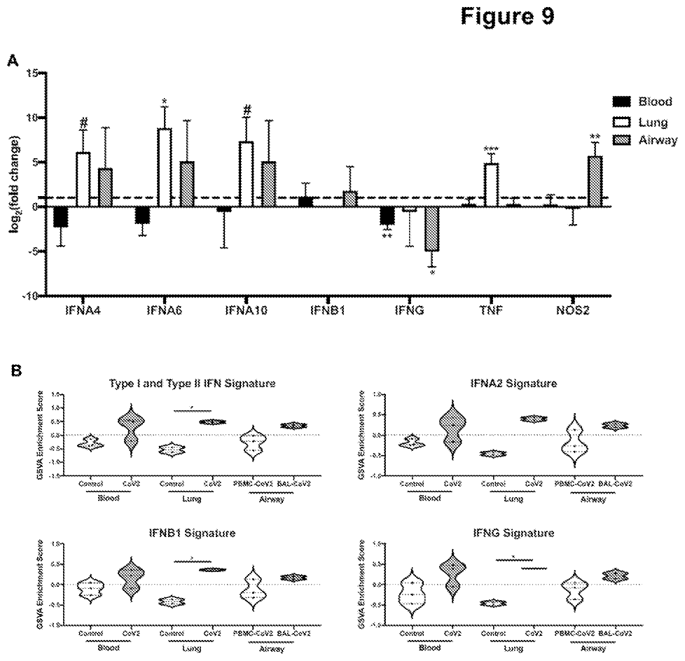

To interrogate pathologic pathways in the 3 compartments, we performed Gene Set Variation Analysis (GSVA) utilizing previously reported interferon response gene (IRG) modules (Catalina et al., 2019), gene modules of inflammatory pathways curated from online databases, and previously defined gene modules characterizing immune and inflammatory cells and processes (Cataline et al. unpublished manuscript) (FIG. 8). This analysis allowed us to identify differences in inflammatory pathways and immune cell types enriched in COVID-19 patients as compared to healthy controls as well those that were differentially enriched between the blood, lung, and airway compartments. Overall, our results revealed increased inflammatory pathway signatures (FIG. 1B), decreased lymphoid cell signatures (FIG. 1C), and increased myeloid cell signatures (FIG. 1D) in COVID-19 patients, a pattern that is consistent with earlier studies (Blanco-Melo et al., 2020; Wen et al., 2020; Xiong et al., 2020). Interestingly, we found important differences in the gene expression profiles between the blood, lung, and airway compartments.

In the blood, inflammatory pathways including the classical and lectin-induced complement pathways and the NLRP3 inflammasome were enriched (FIG. 1B). T cells were significantly decreased in the blood of 2 out of 3 COVID-19 patients and cytotoxic CD8 T cells and NK cells were significantly decreased in all patients. In contrast, we found enrichment of signatures for the CD40 activated pathway and plasma cells only in the blood and not the lung or airway of infected individuals (FIG. 1C). Myeloid populations, which include monocytes and granulocytes, were not enriched in the blood of COVID-19 patients compared to controls (FIG. 1D). However, we did observe a significant increase in the monocyte-specific gene signature as well as polarized M1/M2 macrophages (FIG. 1D).

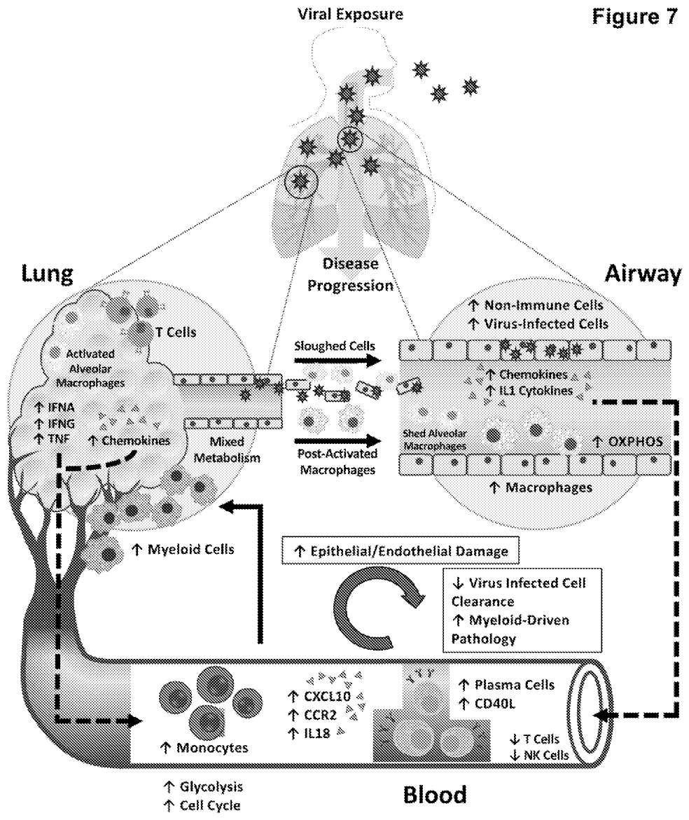

Although, there is conflicting data on the presence of an Interferon Gene Signature (IGS) and whether SARS-CoV2 infection induces a robust IFN response, (Blanco-Melo et al., 2020) we observed increased expression of Type I interferon genes (IFNA4, IFNA6, IFNA10) and significant enrichment of the Type I and Type II IGS specifically in the lung tissue, but not in the blood or airway of COVID-19 patients (FIG. 7; FIG. 1B). We also observed enrichment of other potentially pathologic processes in the lung that were shared with the other compartments, including the classical and alternative complement pathways and NLRP3 inflammasome signatures (FIG. 1B). In the lung tissue, overall T cell signatures were modestly, but not significantly enriched, and activated or cytotoxic cells were not enriched compared to healthy controls suggesting that some T cells were able to enter the infected lung tissue, but were not activated and did not include cells with cytotoxic potential (FIG. 1C). In addition, all myeloid, monocyte, and macrophage populations were enriched as well as neutrophils and granulocytes, consistent with a contribution of multiple myeloid cells types contributing to COVID-19 lung pathology (FIG. 1D; FIG. 8B) (Xu et al., 2020).

In the airway, the classical and alternative complement pathways but not the NLRP3 inflammasome signature were enriched in COVID-19 patients as compared to COVID PBMCs (FIG. 1B). We also observed even greater deficiencies in T cells and cytotoxic cells as compared to the peripheral blood (FIG. 1C). Similar to PBMCs from COVID-19 patients, the general myeloid cell gene signature was not enriched in the airway. In contrast, only the M1/M2 macrophage signatures and not the monocyte signature was significantly enriched compared to the blood (FIG. 1D). As a whole, these results highlight the heterogeneity of immune cell types and resulting inflammatory processes in the peripheral blood, lung tissue, and airways of COVID-19 patients.

Increased Expression of Inflammatory Mediators in the Lungs of COVID-19 Patients

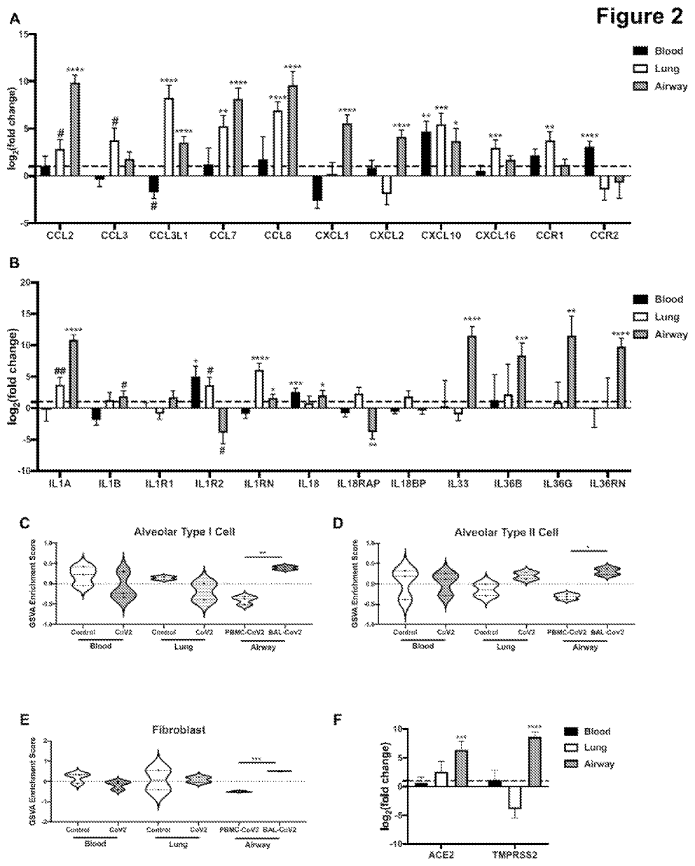

To examine the nature of the inflammatory response in the tissue compartments in greater detail, we examined differential expression of specific genes of interest (FIGS. 2A&B). In the blood, we noted increased expression of the inflammatory chemokine CXCL10, which is induced by IFNγ and involved in the activation and chemotaxis of peripheral immune cells (Lindell et al., 2008). We also found increased expression of the chemokine receptor CCR2, which has been shown to be critical for immune cell recruitment in response to respiratory viral infection (FIG. 2A) (Teijaro, 2015). We found few differentially expressed cytokine genes in the blood, but did observe an increase in expression of the inflammatory IL-1 family member, IL18 (FIG. 2B). Expression of a number of chemokines including ligands for CCR2 were significantly increased in both the lung tissue and airway of COVID-19 patients, including CCL2, CCL3L1, CCL7, CCL8, and CXCL10 (FIG. 2A). Whereas the only IL-1 family cytokines significantly enriched in the lung were IL1A and IL1B, we did observe enrichment of the IL-1 cytokine gene signature by GSVA (FIG. 2B; FIG. 8B). In addition to increased expression of chemokine genes shared with the lung tissue (FIG. 2A), many more pro-inflammatory IL-1 family members including IL18, IL33, IL36B, and IL36G were significantly elevated in the airway and not the lung of COVID-19 patients (FIG. 2B).

Non-Hematopoietic Cells in the BAL Fluid May be Indicative of Viral-Induced Damage in the Lungs

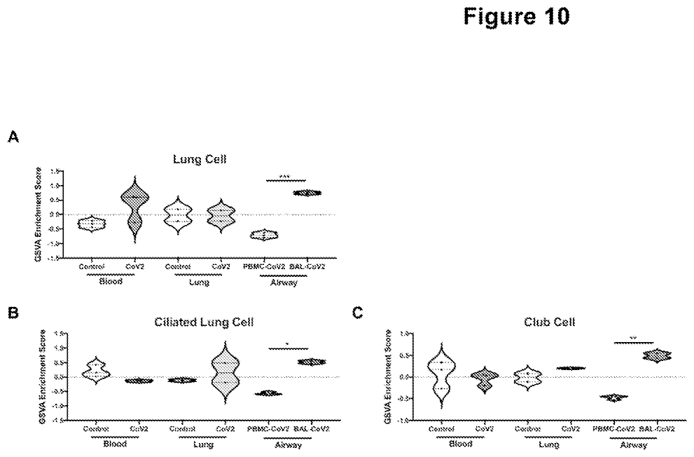

To determine whether viral infection in the lungs and airway resulted in modification of resident tissue populations, we evaluated GSVA enrichment of non-hematopoietic cell type gene signatures including fibroblasts, Type I and Type II alveolar cells, ciliated lung cells, club cells, and a general lung tissue cell signature (FIGS. 2C-2E; FIG. 10). We found that these non-hematopoietic cell signatures were significantly enriched in the airway, but not the lung. Additionally, we only detected increased expression of the viral entry genes ACE2 and TMPRSS2, which are typically expressed on lung epithelium (ref), in the airway of SARS-CoV2-infected patients (FIG. 2F).

Protein-Protein Interaction Metaclusters Identify Myeloid Cells and Metabolic Pathways in Blood, Lung, and Airway of COVID-19 Patients

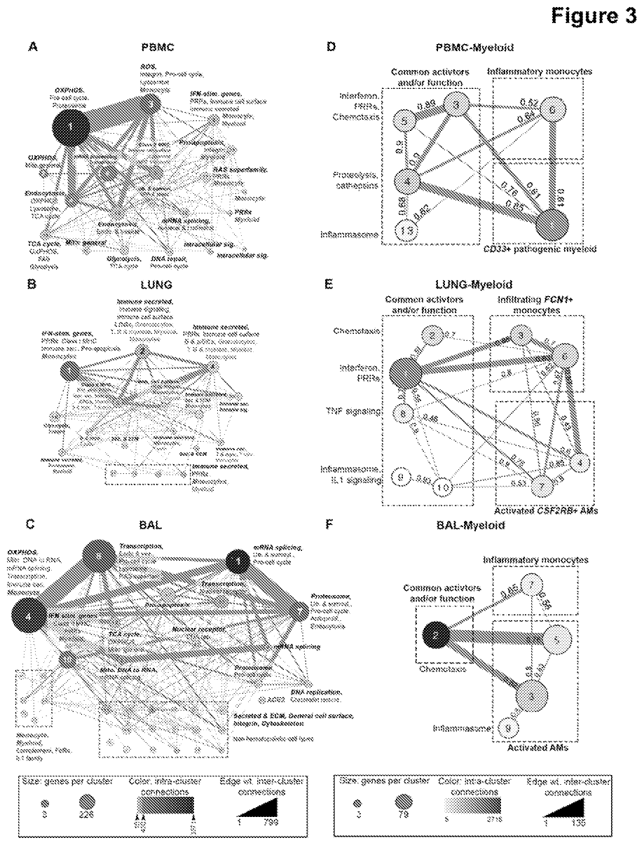

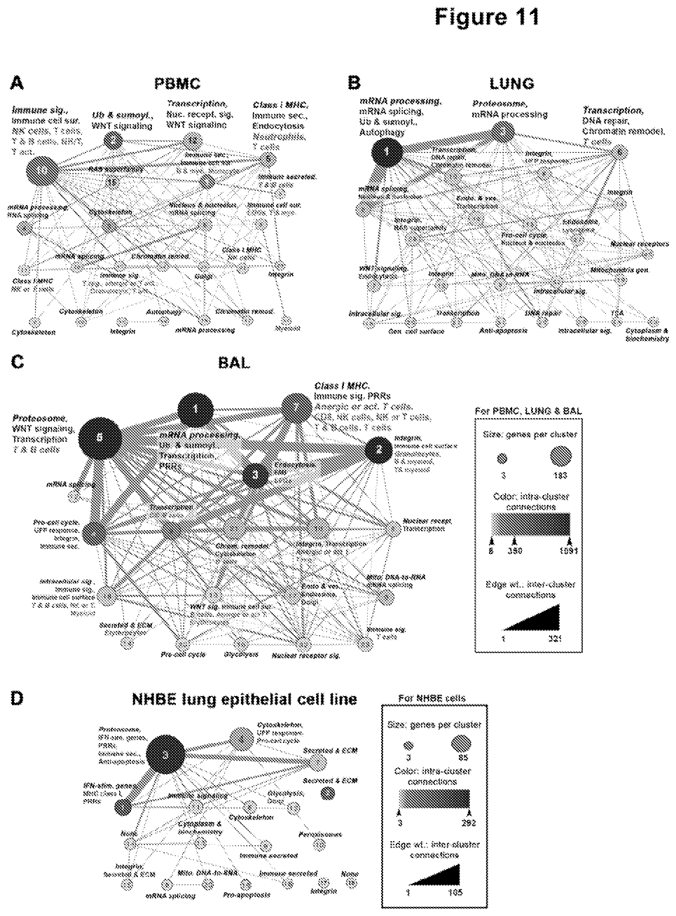

We next sought to utilize an unbiased, clustering approach to assess the relationship between immune cell types and the altered biological processes driving SARS-CoV2-mediated pathology within each tissue compartment. Protein-protein interaction (PPI) networks consisting of DE genes were simplified into metastructures defined by the number of genes in each cluster, the number of significant intracluster connections and the number of associations connecting members of different clusters to each other. Overall, upregulated protein networks from blood, lung, and airway of SARS-CoV2-infected patients identified numerous multifunctional clusters many of which included heterogeneous populations of monocyte and myeloid lineage cells. For example, PBMC cluster 8 was dominated by an inflammatory monocyte population defined by C2, C5, CXCL10, CCR2 and multiple interferon-stimulated genes, whereas cluster 3 contained hallmarks of alternatively activated (M2) macrophages and/or myeloid-derived suppressor cells (MDSCs), including CD33, CD36, CD93 and ITGAM (FIG. 3A). Smaller immune clusters were indicative of specific monocyte/myeloid functions, including inflammasome activation (cluster 54), DAMP activity (cluster 17), the classical complement cascade (cluster 34) and the response to Type II interferons (cluster 32). Myeloid heterogeneity was also reflected in the presence of multiple metabolic pathways, such as enhanced oxidative phosphorylation (OXPHOS) in clusters 1 and 4 linked to M2 macrophages, and glycolysis in clusters 7 and 13 used by inflammatory monocytes. Consistent with our GSVA results, peripheral blood exhibited profoundly suppressed T cells determined by the downregulation of T cell activation markers CD28, LCK and ITK (FIG. 11A).

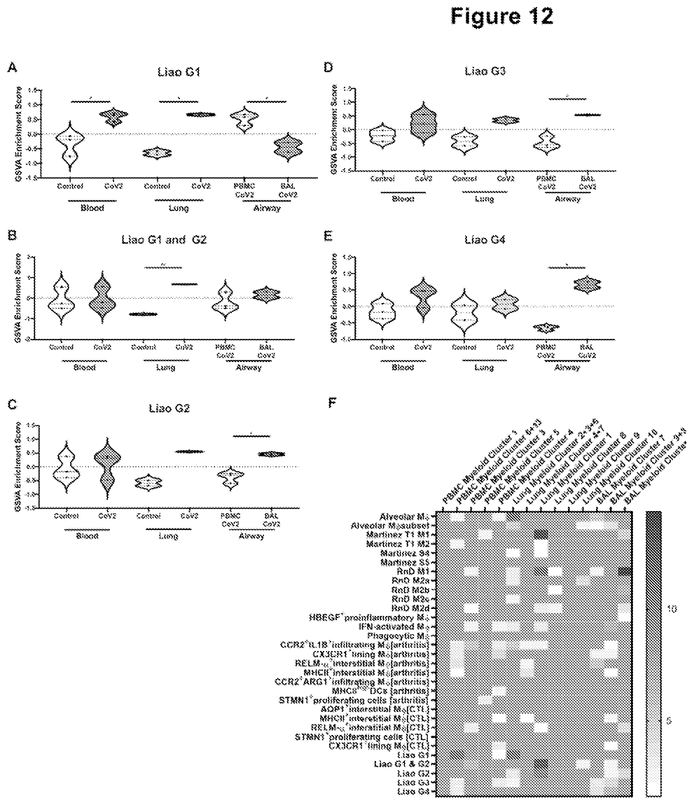

Lung tissue was heavily inflamed exhibiting infiltration of monocyte/myeloid populations with additional infiltration of LDGs, granulocytes, T and B cells. Although distributed among multiple clusters, we observed upregulation of FCN1 (cluster 15), SELL (cluster 14) and S100A8/A9 (cluster 4) which comprise an inflammatory monocyte signature (G1 population) derived from the BAL fluid of COVID patients recently described by Liao et al. (Liao et al., 2020) (FIG. 12A). Metabolic function in the lung was varied, however, upregulated genes segregated with glycolysis, potentially reflecting cellular activation (cluster 18), whereas OXPHOS was predominantly downregulated along with other nuclear processes (transcription and mRNA processing) (FIG. 3B; FIG. 11B).

Finally, the airway was enriched in inflammatory monocytes, mitochondrial function and transcription. Multifunctional cluster 4 was dominated by immune-secreted molecules including numerous chemokine and cytokine receptor-ligand pairs indicative of elevated chemotactic activity, while smaller immune clusters were enriched in classical complement activation (cluster 34), type II interferon (cluster 26) and IL-1 responses (cluster 51). We also observed evidence of recently described alveolar macrophages (AMs; G4 population) specifically in the airway (Liao et al., 2020), although these markers were distributed among multiple clusters, including FABP4 and PPARG in cluster 17, SPP1 and MRC1 in cluster 10, and MARCO and TFRC in clusters 34 and 7, respectively (FIG. 3C, FIG. 12E). The airway was the only compartment with a prominent apoptosis signature located in cluster 47. Consistent with high levels of tissue damage originating from the lung, we observed numerous small clusters reflecting epithelial disruption and non-hematopoietic cell involvement, including clusters 5 and 13 containing multiple intermediate filament keratin genes and cell-cell adhesion claudin genes, respectively. Furthermore, non-hematopoietic cell signatures in the BAL were similar in content to those derived from in vitro COVID19-infected lung epithelium primary cell lines (NHBE) REF (FIG. 11D). Compromised lung function and deep tissue damage was also evident in clusters containing numerous surfactant genes (cluster 43), and the presence of several endothelial (VEGF, PDGFC and DLC1) and fibroblast (ASPN) markers. Similar to the PBMC compartment, T cells were decreased in the airway (FIGS. 11A and 11C).

Myeloid Cell-Derived Metaclusters Define Functional Myeloid Subpopulations within the Blood, Lung, and Airway of Covid-19 Patients

Given the large number of monocyte and myeloid enriched clusters, we next wanted to examine those clusters in greater detail to identify unique myeloid lineage and/or monocyte populations within each tissue compartment. In PBMCs, metaclusters derived from monocyte-enriched clusters revealed new gene modules that were representative of common macrophage function (chemotaxis, proteolysis, etc) as well as two independent monocyte/myeloid subpopulations (FIG. 3D). Cluster 6 contained numerous markers highly reminiscent of classically activated blood monocytes, including C1QA, C1QB, MARCO, TLR4, IRAK1 and was connected to inflammasome genes in cluster 13. Furthermore, clusters 6 and 13 exhibited significant overlap with the inflammatory G1 population defined by Liao et al (2020). In contrast, genes in cluster 1 suggest a second myeloid population characterized by expression of CD33, ITGAM, apoptotic cell clearance (CD93 and MERTK) and high proteolytic capacity. Interestingly, comparison of this population with additional previously defined myeloid populations demonstrated significant overlap with IFN-activated macrophages, CX3CR1+ lining macrophages (arthritis model) and an alveolar macrophage phenotype (FIG. 12F).

Similar to the blood, lung-derived monocyte/myeloid genes segregated into clusters associated with common myeloid-lineage cell functions, such as chemotaxis and pattern recognition, as well as multiple subpopulations (FIG. 3E). Cluster 3 interacted strongly with cluster 6 (interaction score 0.7), containing numerous C-type lectin domain family members involved in cell adhesion, chemotactic receptors (CD74), ligands (CCL8), and FCN1, a recently described marker for highly inflammatory monocytes (G1 population) (Liao et al., 2020). Cluster 6 strongly interacted with pro-migration/chemotaxis cluster 2, and together these clusters exhibited significant overlap with the Liao-defined G1 population, confirming the presence of an infiltrating inflammatory monocyte population in this compartment (FIG. 12F).

Examination of genes in cluster 4 and 7 reveals a potential second myeloid population characteristic of alveolar macrophages (AMs) defined by CSF2RB, which functions as the receptor for GM-CSF, a cytokine that regulates AM differentiation (Joshi et al., 2006; Newton et al., 2016; Suzuki et al., 2014). Interestingly, further characterization of this population indicates significant involvement of the coagulation system indicated by F5, FGG, FGL1, FGL1, SERPINA and SERPINE2.

BAL fluid was also potentially defined by 2 populations, including cluster 7 which had hallmarks of inflammatory/M1 monocytes (MARCO and multiple members of the complement cascade), and AMs in clusters 3 and 5 (FIG. 12F). Cluster 5 was highly enriched in genes related to transcription, but also contained numerous markers for AMs, including APOC1, FABP4 and PPARG and demonstrated significant overlap with the Liao G3 and G4 populations defining “pro-fibrotic” and “lung alveolar macrophages,” respectively (FIG. 12F) (Liao et al., 2020).

Co-Expression Further Delineates Differing Myeloid Gene Expression Between Subpopulations within the Blood, Lung, and Airway of COVID-19 Patients

Both GSVA and PPI networks elucidated the presence of increased myeloid cell populations in all SARS-CoV-2 affected tissues, and PPIs revealed the presence of tissue-specific subpopulations, defined by differing biologic functions. The overlap between many Liao et al. signature genes and the tissue-defined PPI clusters motivated us to further evaluate their enrichment in all tissue compartments. Consistent with PPI clusters, the inflammatory-monocyte like population (G1) was found to be increased in both PBMC and lung, but was significantly decreased in the BAL (FIG. 12A). The G1 and G2 population genes, characterized by predominately interferon-stimulated genes, were increased in the lung (FIG. 12B). Conversely, the “novel intermediate macrophage” population (G2) characterized by inflammatory mediators and chemokines such as CCL2, CCL3 and CCL4, was increased in the BAL, but not the PBMCs (FIG. 12C). Additionally, per patient analyses confirmed the presence of the “pro-fibrotic, SPP1+” macrophage (G3) and “lung alveolar macrophage” populations (G4) in the BAL, although there also may be some evidence of these populations in the lung (FIGS. 12D-12E).

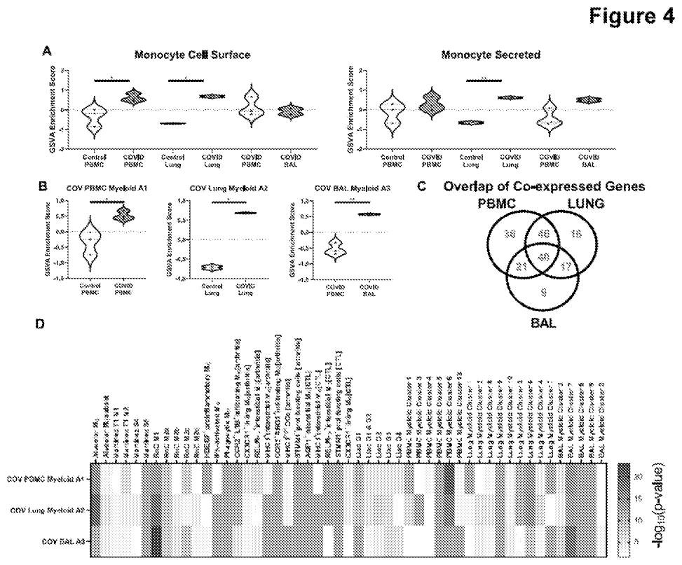

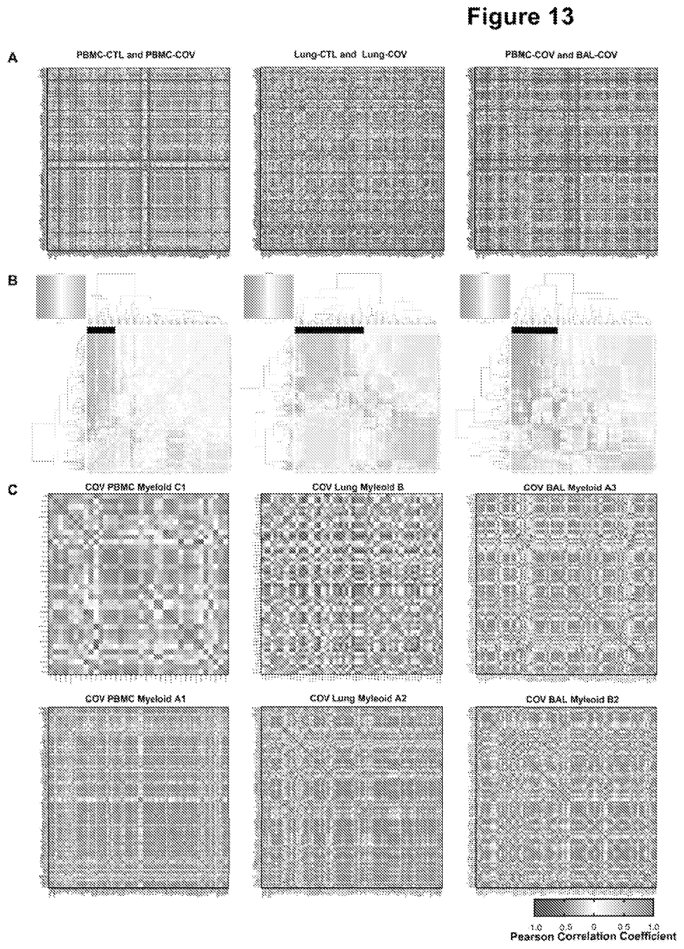

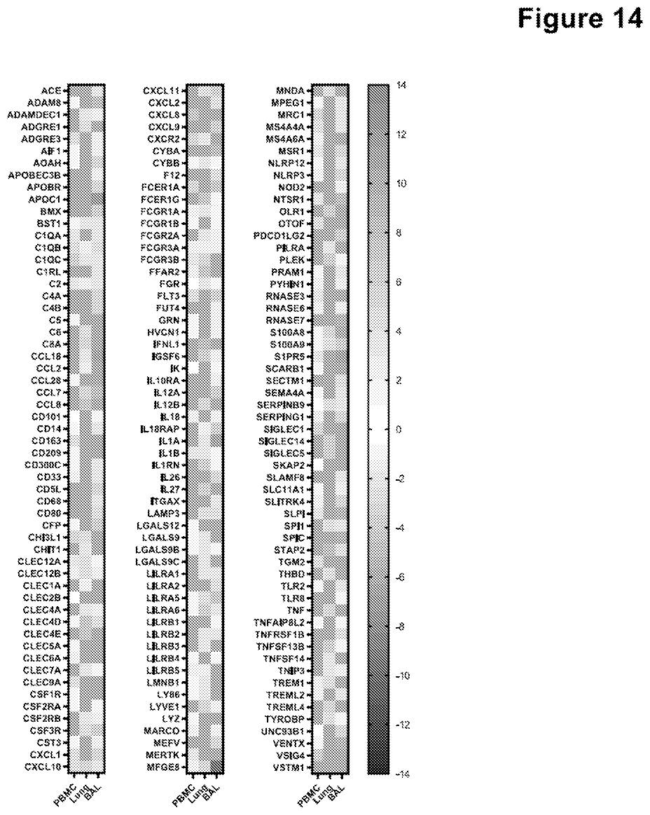

Taken together, evaluation of PPIs and previously published COVID myeloid populations revealed nuances in the myeloid cell populations found among the tissue compartments. We wished to further classify these populations by their expression of traditional myeloid-specific genes including cell surface markers and chemokines. DE interrogation of all possible myeloid cell-specific genes demonstrated further heterogeneity in expression of markers, such as CD14, CD300C, and OSCAR between compartments (FIG. 13). GSVA evaluation of both monocyte cell surface and monocyte secreted gene expression confirmed heterogeneous cell surface markers in the BAL, and increased chemokine secretion in the lung (FIG. 4A). To expand upon the PPI framework, and evaluate how these myeloid-specific genes were expressed in relation to our discovered subpopulations, we utilized co-expression analysis. As such, we evaluated the co-expression of 195 monocyte and myeloid cell genes independently in each tissue compartment. For each gene, a Pearson correlation coefficient was calculated with every other myeloid cell gene for both the samples and controls in each tissue compartment (FIG. 14). The resultant correlation coefficient matrices were then hierarchically clustered into two clusters based upon co-expression (FIG. 14). Each cluster was evaluated in its respective tissue sample and control by GSVA (FIG. 4B). For each compartment, there was a population of genes that were highly co-expressed and altogether increased in each tissue (FIG. 4B). Comparison of the co-expressed genes between each tissue myeloid population shows that many of the increased genes in each tissue are shared (FIG. 4C). The majority of complement genes, including C1QA, C1QB, C1QC C2, C4BPA, and C6 were in included in the 40 genes co-expressed in the increased populations in each compartment. The common co-expressed genes in all tissues also included CCL2, CCL7, CCL8, CXCL10, CCL18, CXCL11, IL18, and TNF. Notably, some of the co-expressed genes unique to the increased COVID lung and BAL populations included CHI3L1, CXCL8, CXCR2, and IL1B, which may be reminiscent of alveolar macrophages. Conversely, these genes were uniquely decreased in the COVID PBMCs.

To characterize the function and nature of these myeloid populations, we again compared them with previously published myeloid signatures including those identified as alveolar macrophages (Reyfman et al., 2019), M1 and M2 populations (Martinez et al., 2006) (Adam's RnD source), and other signatures observed in murine models of inflammatory arthritis (Culemann et al., 2019). Furthermore, we evaluated the overlap between co-expression derived myeloid clusters and those identified using STRING/MCODE PPI in FIG. 3, as well as macrophages clusters recently evaluated in another cohort of COVID BAL fluid (Liao et al., 2020). We observed significant overlap, as determined by Fisher's Exact Test, in many populations (FIG. 4D).

Of the co-expression derived myeloid clusters increased in each tissue compartment, we observed a progression of classically activated, proinflammatory monocytes from the blood into the lung tissue and in some cases getting sloughed off into the airway, as observed by significant overlap with the PBMC myeloid cluster 6, Lung myeloid clusters 3 and 6, Liao et al.'s proinflammatory population (G1), and the CCR2+IL1B+ infiltrating population (FIG. 4D). Interestingly, we observed a similar trend with the CD33+ MDSC-like pathogenic population (PBMC Myeloid Cluster 1), although the overlap was not as significant as the classically activated, proinflammatory monocyte populations. Contrary to these patterns, we did also observe an M1 macrophage population in all three compartments that was most significantly enriched in the COVID BAL fluid (RnD M1). Upon further investigation into the specific overlapping M1 genes, this observed enrichment was primarily due to the presence of chemokines, proinflammatory cytokines, and activation marker CD80. Additionally, we identified alveolar macrophages in all compartments but primarily in the lung, characterized by inhibitory M2-like gene transcripts, alarmins, chemokines, Fc fragments of the IgG receptor, and CD11c. Despite identification of myeloid populations with features of multiple previously described subtypes, the increased myeloid co-expression derived populations were predominantly proinflammatory and were characterized by pathogenic, activated phenotypes.

Linear Regression Confirms Functional Associations with Myeloid Subpopulations

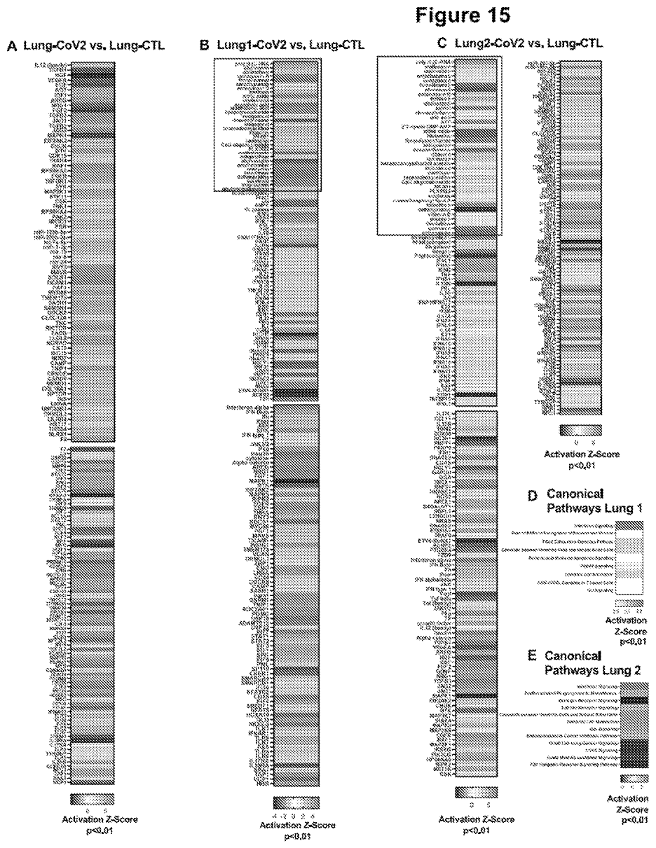

To confirm the function of these myeloid populations in the pathology of SARS-CoV-2, we performed linear regression analyses between myeloid GSVA scores and functional or signaling pathways. Earlier DE functional enrichment and MCODE/STRING analyses suggested a predominant role for metabolism, particularly glycolysis and the TCA cycle, in PBMCs, as well as OXPHOS in all three tissue compartments. We therefore evaluated metabolism in each compartment using GSVA (FIG. 15). We found that the TCA cycle was significantly increased in PBMCs, whereas OXPHOS is significantly increased in the BAL (FIGS. 15C-15D). Additionally, pro-cell cycle genes were increased in PBMCs and pro-apoptosis genes were decreased. We thus evaluated apoptosis and cell cycle on an individual patient basis in each of the compartments using GSVA (FIG. 16). Furthermore, both TNF signaling and complement proteins have been noted as relevant to COVID pathology, and these were evaluated by GSVA as well (FIG. 1).

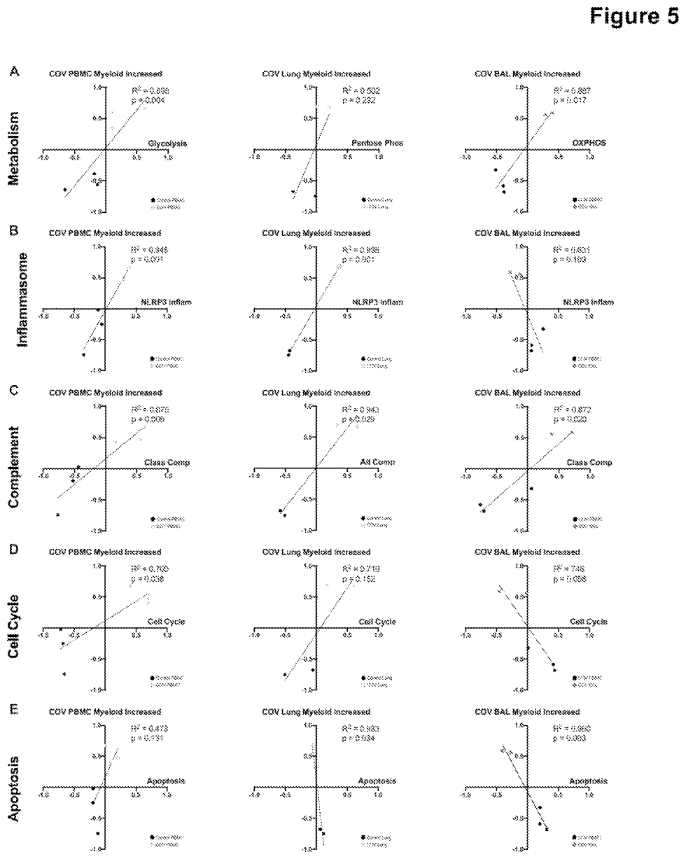

Linear regression analyses between GSVA scores for the myeloid cell populations and metabolic, functional, and signaling pathways in each compartment further demonstrated differences among the myeloid cell populations. The myeloid cell population in the PBMCs was found to be highly glycolytic, whereas there was no significant change to metabolism detected in the lung, and the population in the BAL was reliant on OXPHOS (FIG. 5A). Although myeloid cell signatures were associated with the NLRP3 inflammasome in the PBMCs and lungs, this association was not found in the BAL (FIG. 5B). The classical complement cascade was significantly correlated with the increased myeloid cells in both PBMCs and BAL, whereas the alternative complement cascade was significantly correlated with the myeloid cells in the lung (FIG. 5C). The myeloid cells in the PBMCs were also significantly correlated with the cell cycle, but this may be more evident of plasma cells in the blood (FIG. 5D). Additionally, the lung and BAL myeloid populations were negatively correlated with apoptosis (FIG. 5E). As a whole, our analyses reveal the heterogeneity of myeloid cell populations in the blood, lung, and airway of COVID-19 patients in their metabolic and overall inflammatory status, which has critical implications for the role (s) these populations play in COVID-19 pathogenesis.

IPA Confirms Metabolic Activity and Viral, Innate Immunity as Targetable Pathways in COVID-19 Patients

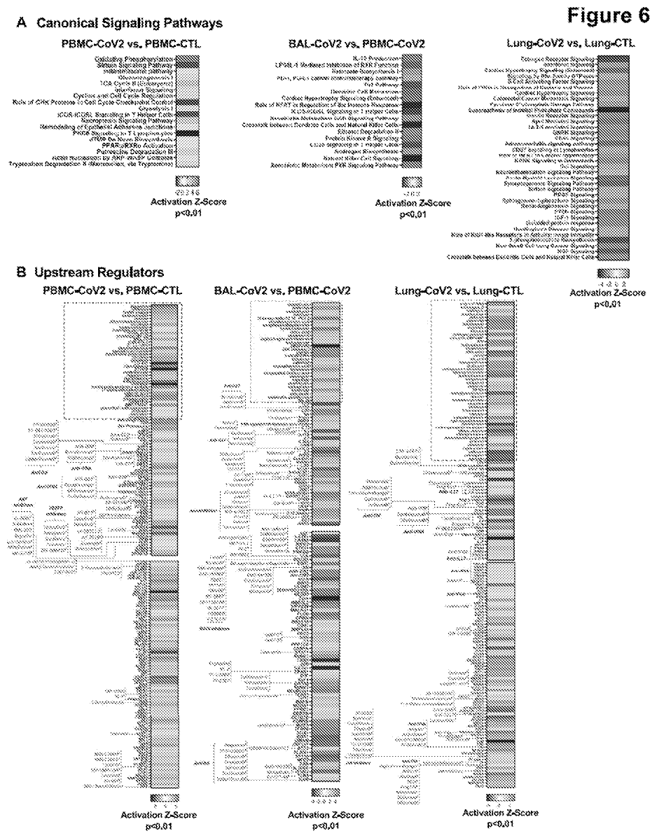

To further inform the biology of SARS-CoV-2-infected patients, we conducted pathway analysis on DEGs from each of the peripheral blood, lung, and airway compartments using IPA canonical signaling pathway and upstream regulator analysis functions (FIG. 6). Gene expression fold changes were used to inform statistical overlap with known signaling pathways and molecules upstream of DEGs in SARS-CoV-2. In general, interferon signaling, the inflammasome, and other components of antiviral, innate immunity were reflected by the disease state gene expression profile compared to healthy controls (FIG. 6A). In addition, metabolic pathways including oxidative phosphorylation and glycolysis were significantly activated in SARS-CoV-2 PBMCS compared to controls. Though no differences were found between the PBMCs and BALF in these particular pathways, interestingly, xenobiotic metabolism was predicted to be significantly more activated in the airway indicating a cellular defense response.

Upstream regulators predicted to mediate responses to the virus in each compartment indicated uniform involvement of proinflammatory cytokines with type I interferon regulation dominant in the diseased lung (FIG. 6B). Notable upstream regulators of SARS-CoV-2 peripheral blood included IFNA, IFNG, multiple growth factors and ligands, HIF1A, CSF1 and CSF2; however, when compared to the BALF, upstream regulators of the peripheral blood were more indicative of the presence of lymphocytes. Evidence of proinflammatory cytokine signaling by IL17 and IL36A were predicted in COVID-19 lung and airway compartments. Whereas the BALF DEG profile indicated upstream regulation by both inflammatory and inhibitory cytokines, including IL6 and IL13 and IL10, respectively, the COVID-19 lung upstream regulators were markedly proinflammatory, including, NFκB, IL12, TNF, IL1B, and multiple type I interferons. Small molecules, drugs, and compounds that were predicted as upstream regulators or matched to targets indicate unique therapeutic possibilities in each tissue compartment. Of note, anti-IL17, anti-IL6, anti-IL1, anti-IFNA, anti-IFNG, and anti-TNF treatments were predicted as antagonists of SARS-CoV-2 biology. Steroids were predicted to revert the gene expression profile in the diseased lung, but were predicted as upstream regulators of COVID-19 PBMCs. Finally, chloroquine was additionally predicted to revert the SARS-CoV-2 transcription profile in the lung.

Discussion

In the present study, we utilize multiple orthogonal bioinformatics approaches to analyze differential gene expression from the blood, lung, and airway of COVID-19 patients. As a whole, our results are in agreement with the current understanding of SARS-CoV2 pathogenesis, which generally involves lymphopenia accompanied by increased activation of pro-inflammatory pathways and myeloid cells (Tay et al.). In particular, we found that DE genes from COVID-19 patients were enriched in gene signatures of interferon, complement pathways, and the inflammasome, which would be expected to initiate a robust and systemic response to infection. We also utilized various approaches to derive and characterize the expanded myeloid cell subpopulations in COVID-19 patients. These observed increases in inflammatory pathways and myeloid cell subsets are likely reflective of a dysregulated immune response that contributes to lung damage, and in severe cases, to multi-organ failure and overall poor health outcomes in COVID-19 patients. Furthermore, by comparing DE results from multiple compartments (the blood, lung, and airway) in COVID-19 patients, we have developed a model of the systemic pathogenic response to SARS-CoV2 infection (FIG. 7).

In our proposed model, after viral exposure, SARS-CoV2 enters the host airway and infects alveolar epithelial cells, which express the virus entry receptors ACE2 and TMPRSS2. In the lung tissue, we observed increased expression of Type I interferon genes, enrichment of Type I and Type II interferon gene signatures, and expression of IFN-stimulated genes including TNF and NOS2. Type I interferons are critical components of the host response to viral infection through inducing the expression of anti-viral genes and direct or indirect immune cell activation and, thus, are targets of immune evasion tactics by coronaviruses including SARS-CoV and MERS-CoV (Newton et al., 2016; Tay et al.). Type I interferons can be produced by virus-infected cells, or cells that have detected viral infection (Goritzka et al., 2015). Therefore, interferon production in the lung of COVID-19 patients is likely initiated by infected alveolar cells and propagated by activated alveolar macrophages, leading to the production of IFNγ and other pro-inflammatory mediators (Darwich et al., 2009; Newton et al., 2016). To date, reports have been divided over whether an interferon response is present in COVID-19 patients in that some reports cite a poor interferon response (Blanco-Melo et al., 2020), some observe a robust interferon response that increases with disease severity (Wei et al., 2020), and still others have found heterogeneity in whether patients exhibit an interferon response to SARS-CoV2 infection (Trouillet-Assant et al., 2020). Therefore, the interferon response to SARS-CoV2 requires further study and consideration when developing treatment strategies for individual patients.

Signals from the lung including interferon or other pro-inflammatory mediators from infected cells disseminate into the peripheral blood to launch a systemic inflammatory response. In the periphery, inflammatory mediators from the lung typically promote activation and migration of myeloid cells, NK cells, and adaptive immune cells including T and B cells, which can differentiate into effector CD8 T cells and antibody-producing plasma cells (Newton et al., 2016). We found significant deficiencies in gene signatures of T cells, NK cells, and cytotoxic cells, which include activated CD8 T cells and NK cells, in the peripheral blood of COVID-19 patients, which is consistent with clinical and analytical evidence of lymphopenia following SARS-CoV and SARS-CoV2 infection (Chen et al., 2020b; He et al., 2005; Qin et al., 2020; Xu et al., 2020). In contrast to T and NK cells, we observed increased evidence of B cell activation through CD40/CD40L and an increased plasma cell signature in PBMCs of COVID-19 patients. This result suggests that while effector CD8 T cells are deficient, that helper T (Th) cells are present and able to promote B cell activation, the generation of antibody secreting plasma cells, and an antibody-mediated immune response. However, whether a virus-specific antibody response is beneficial to recovery from SARS-CoV2 infection is unclear (Wu et al., 2020). Evidence from SARS-CoV infection suggests that the quality of the antibody produced is critical to mounting an effective anti-viral response. Low quality, low affinity antibody responses may have pathological consequences including promoting lung injury in some patients, although it is unknown if this occurs in SARS-CoV2 infected individuals (Iwasaki and Yang, 2020; Liu et al., 2019).

The predominant populations of immune cells we found to be enriched and activated in COVID-19 patients were myeloid cells and, in particular, subsets of inflammatory monocytes and macrophages, which differed between the blood, lung, and airway compartments. In the peripheral blood, we found significant enrichment of monocytes including classically activated inflammatory monocytes as well as another subset characterized by expression of CD33. This CD33⁺ myeloid subset appeared to be an alternatively activated population reminiscent of previously characterized IFN-activated macrophages and alveolar macrophages (AMs), which may represent an activation state specific to stimuli arising from the SARS-CoV2-infected lung. Myeloid cells enriched in the blood of COVID-19 patients were also correlated with pro-cell cycle and glycolysis gene signatures indicative of a metabolic status associated with pro-inflammatory M1 macrophages (Viola et al., 2019). In support of the presence of increased pro-inflammatory monocytes/macrophages, we also found significant increases in the expression of the pro-inflammatory IL-1 family member IL18, the interferon-induced chemokine CXCL10, and the chemokine receptor CCR2, all of which have cited roles in the response to respiratory virus infection (Tang et al., 2005; Teijaro, 2015; Wang et al., 2004).

In the lung tissue, we observed increased expression of a number of chemokine ligands for CCR2 (CCL2, CCL7, CCL8) and other receptors (CCL3, CXCL10, CXCL16), which would promote the mobilization of activated immune cells to the locus of infection. While in the blood, we found evidence of increased monocyte/macrophage populations, in the lung tissue, we also observed enrichment of gene signatures of other myeloid cells including neutrophils and low density granulocytes (LDGs), which we could not assess in the blood due to the process of isolating PBMCs. The role of neutrophils and LDGs in COVID-19 pathogenesis has been poorly characterized although they have been found in increased numbers and associated with poor disease outcome in COVID-19 patients (Huang et al., 2020). In addition, some have proposed that the formation of neutrophil extracellular traps (NETs) contributes to increased multi-organ damage and risk of death from SARS-CoV2 infection (Barnes et al., 2020; Thierry and Roch, 2020).

Monocyte/macrophage subsets in the lung of COVID-19 patients were characterized as infiltrating inflammatory monocytes and activated AMs, which exhibited a mixed metabolic status suggestive of different states of activation. Infiltrating monocytes from the peripheral blood appeared to be further activated in the lung tissue as evidenced by enhanced expression of alarmins and markers of highly inflammatory monocytes previously characterized in severe COVID-19 cases (Liao et al., 2020). In particular, we observed increased expression of IL-1 family members, most notably ILIA and enrichment of a pro-inflammatory IL-1 signature in the lung of COVID-19 patients. We also found evidence of a population of AMs previously defined in the BAL of COVID-19 patients (Liao et al., 2020). Alveolar macrophages are typically suppressed at steady state, but adopt a pro-inflammatory phenotype after viral infection in order to assist in phagocytosis and clearance of virus (Newton et al., 2016). However, we observed that this AM population also clustered with genes involved in the coagulation system, which is consistent with observations of procoagulant AM activity in ARDs (Tipping et al., 1988). As pulmonary thrombosis has been associated with poor clinical outcomes in COVID-19 patients, this result suggests that AMs in SARS-CoV2-infected lung tissue may be detrimental to host recovery from disease (Wichmann et al., 2020).

While we observed significant decreases in T cells in the blood of COVID-19 patients, they appeared to be increased in the lung tissue. However, upon closer examination, we noted that there was no enrichment of activated T cells or of cytotoxic CD8 T cells. This indicated that while T cells may be able to enter the infected lung tissue, they are not activated and thus lack the ability to clear virus-infected cells and resolve the inflammatory response. In cases of macrophage activation syndrome (MAS) defects in cytotoxic activity of CD8 T cells and NK cells result in increased cell-cell contacts, enhanced immune cell activation, and intensified production of pro-inflammatory cytokines. Many of the pro-inflammatory cytokines associated with MAS were also expressed in our COVID-19 patient samples, predominantly in the lung tissue and airway compartments, including IL1, IL18, and IL33 (Crayne et al., 2019; McGonagle et al., 2020). Thus, it is likely that the lack of activated CD8 T cells and NK cells and subsequent failure to clear virus-infected cells, is a major contributor to the dysregulated, macrophage-driven pathologic response to SARS-CoV2 observed in COVID-19 patients.

As disease progresses, increased infiltration of pro-inflammatory immune cells and release of inflammatory mediators in the lung is emulated by the presence of immune cells in the airway, which can be sampled through the BAL (Heron et al., 2012). Thus, the contents of the BAL fluid acts as a diagnostic marker or sensor for what is occurring in the lung tissue. In the airway, we detected gene signatures of various post-activated macrophage subsets including inflammatory M1 macrophages, alternatively activated M2 macrophages, and activated alveolar macrophages that had either migrated or been shed from the lung tissue. In conjunction with the presence of inflammatory macrophages, our results revealed a prominent IL-1 family signature in the airway of COVID-19 patients above what we observed in the blood or lung tissue and, in particular, an over 10-fold increase in expression of the alarmins ILIA and IL33 (Jing et al., 2016; Schmitz et al., 2005). These alarmins act as danger signals released by dead or dying cells to promote immune cell activation and therefore, their presence in the airway is likely indicative of an increase in virally infected lung cells. As SARS-CoV2 continues to propagate and viral clearance is impaired by a lack of cytotoxic activity, these alarmins promote inflammation and prevent resolution of the immune response in COVID-19 patients. Thus, this result further supports the notion that IL-1-mediated inflammation plays a critical role in COVID-19 pathogenesis. Expression of myeloid cell genes in the airway also correlated with a signature of oxidative metabolism, which is characteristic of M2 macrophages and typically associated with control of tissue damage (Viola et al., 2019). However, in the context of pulmonary infection, polarization of alveolar macrophages toward an anti-inflammatory M2 phenotype was found to promote continued pathogenesis, suggesting that these macrophages may not be effective mediators of anti-viral immunity (Allard et al., 2018).

We found increased enrichment of lung epithelial cells as well as increased expression of the viral entry receptors ACE2 and TMPRSS2, which are expressed by these cells, exclusively in the airway of COVID-19 patients. The presence of non-hematopoietic cells such as lung epithelium in the BAL is indicative of damage to the lung tissue as dead or dying cells are being sloughed off into the airway and suggests that localized inflammation as a result of enhanced immune cell infiltration promotes significant damage to the lung tissue. Furthermore, the lack of cytotoxic cells and thus, the inability to clear these virus-infected lung epithelial cells in the airway likely accounts for the increased presence of post-activated macrophages and high expression of pro-inflammatory IL-1 family members we observed in the BAL of COVID-19 patients. However, in contrast to previous characterizations of both SARS-CoV and SARS-CoV2 infection, our analysis did not indicate the presence of overwhelming pro-inflammatory cytokine production or “cytokine storm” (Tay et al.). Rather our data suggests that the increased numbers, overactivation, and potentially heightened pathogenicity of monocyte/macrophage populations are the main drivers of the dysregulated immune response and resulting tissue damage in COVID-19 patients.

Big Data Analyses Facilitate Drug Prediction

In the absence of effective antiviral treatment and/or a SARS-CoV2-specific vaccine, disease management is reliant upon supportive care and therapeutics capable of limiting the severity of clinical manifestations. Using empirical evidence as a guide, the current approach has been successful in identifying “actionable” points of intervention in an unbiased manner and in spite of formidable patient heterogeneity. Analyses presented here support several recent reports highlighting COVID19 infection-related increases in secreted factors, particularly IL6 and TNF, both of which function as predictors of poor prognosis (Pedersen and Ho, 2020; Voiriot et al., 2017), as well as complement activation (Gao et al., 2020; Huang et al., 2020; Mehta et al., 2020). Accordingly, anti-IL6 therapies including sarilumab, tocilizumab and clazakizumab, as well as biologics targeting terminal components of the complement cascade, such as eculizumab and ravulizumab, are in various clinical trial phases for treating COVID19 associated pneumonia. Candidate TNF blockers such as adalimumab, entanercept and many others, represent additional options for inhibiting deleterious pro-inflammatory signaling. Interestingly, numerous tubulin inhibitors were identified among the complied list of drugs counteracting infection-induced genomic changes; it is therefore of note that clinical trials involving colchicine, an antimitotic drug that binds soluble tubulin, are currently underway, providing further validation for the unbiased drug-prediction methodology presented here. Our analyses also point to the likely involvement of pro-inflammatory IL1 family members especially in the lung, suggesting anti-IL1 interventions, including canakinumab and anakinra, may be effective in preventing acute lung injury.

Overall, this report establishes the predominance of inflammatory monocyte/myeloid lineage cells in driving disease pathology and suggest therapies effective at blocking myeloid cell recruitment or forcing repolarization may prevent disease progression. CCL5 (RANTES) is a potent leukocyte chemoattractant that interacts with multiple receptors, including CCR1 (upregulated in the blood, lung and airway), and CCR5 (upregulated in the airway). Disruption of the CCR5-CCL5 axis was recently tested using the CCR5 blocking monoclonal antibody leronlimab in a small compassionate use trial with promising preliminary results (Pattterson et al.). Similarly, CD74 which functions as the receptor for the pro-inflammatory cytokine macrophage migration inhibitory factor (MIF), was upregulated specifically in the lung and represents an attractive target for intervention by imalumab, an anti-MIF monoclonal antibody.

It has also been observed that COVID19 may predispose patients to thromboembolic disease (Klok et al., 2020; Middeldorp et al., 2020). Indeed, the gene expression analyses presented here showing altered expression of coagulation factors and fibrinogen genes suggests dysfunction within the intrinsic clotting pathway. These findings, together with evidence of excessive inflammation and complement activation, may contribute to the systemic coagulation underlying the remarkably high incidence of thrombotic complications observed in severely ill patients, thereby reinforcing recommendations to apply pharmacological anti-thrombotic medications.

Finally, there has been much recent discussion concerning the use of anti-rheumatic drugs for managing COVID19. In fact, chloroquine (CQ) was one compound predicted as an upstream regulator with potential phenotype-reversing properties. In vitro experiments examining the anti-viral properties of CQ and its derivative hydroxychloroquine (HCQ) were effective in limiting viral load, however the efficacy of these drugs in clinical trials has been less clear. Questions about drug timing, dosage and adverse events have all called into question the use of these drugs for COVID19 patients.

Using systems and methods of the present disclosure, a treatment may be selected for an individual based on data analyses of biological samples obtained from the individual, in order to treat or manage a COVID19 disease state or condition of the individual. For example, a targeted treatment for the blood may be selected for an individual based on data analyses of biological samples obtained from the individual, in order to treat or manage a COVID19 disease state or condition of the individual. For example, a targeted treatment for the lungs may be selected for an individual based on data analyses of biological samples obtained from the individual, in order to treat or manage a COVID19 disease state or condition of the individual. For example, a targeted treatment for the airways may be selected for an individual based on data analyses of biological samples obtained from the individual, in order to treat or manage a COVID19 disease state or condition of the individual. In some embodiments, the treatment may be selected from among a plurality of different treatments for an individual based on data analyses of biological samples obtained from the individual, in order to treat or manage a COVID19 disease state or condition of the individual.

Weighted Gene Co-Expression Network Association

DEseq2 normalized log 2 transformed RNAseq gene counts for CRA2390 and GSE147507 datasets were used as inputs to WGCNA (V1.69). Adjacency co-expression matrices for all genes in a given set were calculated by Pearson's correlation using signed network type specific formulae. Blockwise network construction was performed using a soft threshold power value of 30 in order to preserve maximal scale free topology of the networks. Resultant dendrograms of correlation networks were trimmed to isolate individual modular groups of genes, labeled using semi-random color assignments, based on a detection cut height of 1, with a merging cut height of 0.2, with the additional use of a partitioning around medoids function. The module eigenegene (ME) vector per sample was calculated as the first principle component of the module's gene expression counts. Module correlations to cohort were calculated using Pearson's r against MEs, defining modules as either positively or negatively correlated as a whole by averaging constituent sample ME correlations to cohort. The strength of module representation was established by inspecting the number of members of the disease or healthy samples contributing to the overall average ME correlation to disease state. Majority modules highly representative of their cohort were those where more than half of the cohort constituent MEs were correlated in the same direction and general scale to cohort. Quality majority modules were those with the additional requirement that the opposing cohort correlations were all running in the opposite direction. Minority quality disease state modules were considered as being more representative of genetic expressions unique to patient rather than cohort. Module membership statistics were calculated including kIM, a measurement of intramodular connectivity of each gene's expression values across samples to neighboring module genes, kME, the correlation of each gene to its containing module eigengene, and general correlation of gene expression values to cohort. Hub genes were considered as those with the highest kIMs, and as a general rule also had the highest kMEs. Complete composite module preservation statistics were calculated using WGCNA's modulePreservation function through 200 permutations of the three data sets independently tested against each other as either a reference or test set. The Z summary statistic was selected as the global index of module preservation and is a composite of seven density and connectivity preservation statistics. All constituent statistics were retained for future granular analysis. Modules were considered as moderately preserved between reference-test network pairs as those with a Z summary statistic between 5 and 10, and as highly preserved as those with a Z summary score above 10. Preservation median rank was calculated to scale module preservation to module size. Gene symbols common between moderately and highly preserved modules in each reference-test pair were selected for ensuing GSVA enrichment analysis.

MEGENA Gene Co-Expression Network Association

The MEGENA (Multiscale Embedded Gene Co-expression Network Analysis) package (v1.3.7) was applied to reconstruct co-expression networks, as described by, for example, [Won-Min Song and Bin Zhang (2018). MEGENA: Multiscale Clustering of Geometrical Network. R package version 1.3.7. CRAN.R-project.org/package=MEGENA], which is incorporated by reference herein in its entirety. The DEseq2 normalized log 2 transformed RNAseq gene counts used for WGCNA analysis were used for initial MEGENA correlation calculation. Gene expression rows with a standard deviation of less than 0.2 were discarded. The correlations between all remaining gene pairs was calculated using Pearson's correlation coefficient (φ using MEGENA's calculation correlation function. After calculation of true pairwise ρ values, a permutation procedure determined the necessary ρ value necessary to achieve a false positive rate (FPR) of 0.05. The gene expression matrix was shuffled through 10 permutations and ρ coefficient results were pooled. Gene correlations greater than an FPR of 0.05 were discarded. An FDR threshold of 15% was additionally enforced to reduce type-one errors. Thresholded FDR correlations were submitted to the MEGENA package for generation of a planar filtered network (PFN) of genes mapped to each other through network connectivity strength. Briefly, gene pairs were first ranked based on expression similarity and then iteratively tested for planarity to expand the PFN, while favoring those pairs with larger similarities. Multi-scale module structures were generated through Multiscale Clustering Analysis (MCA) by clustering initial connected components of the PFN as parent clusters, with clustering repeated in an iterative fashion down through module lineages until no meaningful descendent modules remained to split. A minimum module size of 20 genes was enforced throughout. Multiscale Hub Analysis (MHA) was performed to detect significant hubs of individual clusters and across a, characterizing different scales of organizations in the PFN with emphasis given to multiscale hubs. Significant MEGENA modules were selected that showed a significance of compactness by p<0.05. Analogous to WGCNA, Cluster-Trait Association Analysis (CTA) was performed by performing principle component analysis (PCA) on each module and correlated to cohort, with correlation significance set to Fisher's adjust p value <0.05. First-generation modules (founding members of their lineage) were examined for various cell and pathway signature gene set variation analysis (GSVA) enrichments, with significant enrichments as those with a Hedge's corrected adjusted p value <0.05. MEGENA modules were coerced into WGCNA's module preservation function and analyzed as before by considering overlapping genes from reference-test network pairs with a mutual minimum composite Z summary statistic of 5. MEGENA modules were renamed to indicate their pedigree and networks were visualized using sunburst diagrams to depict module lineages. Some sunbursts were colored using majority WGCNA color assignment to elucidate differences in module creation between the MEGENA and WGCNA approaches. Some were colored using majority cell signature enrichment to demonstrate inheritance of cell signature through module lineage, and others colored with gene expression log-fold change. Heatmaps of log 2 transformed module gene expression were inspected to establish how well modules represented their cohort, similar to majority and minority quality WGCNA modules. Heatmaps were also generated of various module GSVA cell signature enrichments to further curate modules of immunological and other interests.

Module Gene Co-Expression Network Visualization

Modules showing high enrichment of cell signatures of interest were selected for additional enrichment analysis, pathway annotation, and network visualization. Module official HGNC gene symbols were imported into Cytoscape (v3.8.0) through its STRING (v11) protein query set to a confidence score cutoff of 0.9 with zero allowed maximum additional interactions. Cytoscope is described by, for example, [Shannon P, Markiel A, Ozier O, Baliga N S, Wang J T, Ramage D, Amin N, Schwikowski B, Ideker T. Cytoscape: a software environment for integrated models of biomolecular interaction networks. Genome Res, 13:11 (2498-504). 2003 November PubMed ID: 14597658], which is incorporated by reference herein in its entirety. String is described by, for example, [Szklarczyk D, Gable A L, Lyon D, Junge A, Wyder S, Huerta-Cepas J, Simonovic M, Doncheva N T, Morris J H, Bork P, Jensen L J, von Mering C. STRING v11: protein-protein association networks with increased coverage, supporting functional discovery in genome-wide experimental datasets. Nucleic Acids Res. 2019 January; 47:D607-613], which is incorporated by reference herein in its entirety. Edges (connections) between gene nodes were weighted as the STRING connection strengths. Genes with less than 3 connections to adjacent nodes were discarded. MCODE clustering was calculated on the whole network with a degree cutoff of 2 and clusters found allowing haircut and fluff, and some visualizations colored by MCODE cluster, as described by, for example, [Bader G D, Hogue C W. An automated method for finding molecular complexes in large protein interaction networks. BMC Bioinformatics, 4: (2). 2003 Jan. 13. PubMed ID: 12525261], which is incorporated by reference herein in its entirety. AMPEL cell signature annotations were merged to the nodes data table and used to adjust node color, border color, and sizes to call out genes of interest. Various attribute and network cluster algorithms from the Cytoscape clusterMaker app were applied to help simplify visualizations, including GLay community clustering, as described by, for example, [Morris J H, Apeltsin L, Newman A M, Baumbach J, Wittkop T, Su G, Bader G D, Ferrin T E. clusterMaker: a multi-algorithm clustering plugin for Cytoscape. BMC Bioinformatics, 12: (436). 2011 Nov. 9. PubMed ID: 22070249], which is incorporated by reference herein in its entirety. BiNGO was used to query various GO (gene ontology) pathways to further elucidate network relations to biological processes, as described by, for example, [Maere S, Heymans K, Kuiper M. BiNGO: a Cytoscape plugin to assess overrepresentation of gene ontology categories in biological networks. Bioinformatics, 21:16 (3448-9). 2005 Aug. 15. PubMed ID: 15972284], which is incorporated by reference herein in its entirety. Clusters from various modules were visualized together and examined for interconnectedness. Meta clusters were created by combining genes with similar functional annotation into composite nodes, with edges between them weighted as the total number of MCODE connections between constituent genes.

Derivation of Lung Tissue Cell Populations for GSVA

Nine single-cell RNA-seq lung cell populations (AT1, AT2, Ciliated, Club, Endothelial, Fibroblasts, Immuno Monocytes, Immuno T Cells, and Lymphatic Endothelium) were downloaded from the Eils Lung Tissues set (www.biorxiv.org/content/10.1101/2020.03.13.991455v3) accessed by the UC Santa Cruz Genome Browser (eils-lung.cells.ucsc.edu). Genes occurring in more than one cell type were removed. Additionally, genes known to be expressed by immune cells were removed. The Immuno Monocyte and Immuno T cell categories were not employed in further analyses.

Derivation of Co-Expressed Myeloid Subpopulations in Each Compartment

Co-expression analyses were conducted in R. Sample (control and patient) log 2 expression values for each gene of the 221 identified monocyte/myeloid cell genes in were analyzed for their Pearson correlation coefficient in each tissue compartment (PBMC, lung, BAL) using the Cor function. Of note, only 195 of 221 genes had changes in gene expression in at least one tissue by RNA-seq, and these genes are shown. Resulting correlation matrices were hierarchically clustered by their Euclidian distance into 2 clusters (k=2) using the heatmap.2 function in R. This resulted in 2 monocyte/myeloid co-expressed clusters in each compartment. The co-expressed myeloid populations in each compartment were then evaluated by GSVA.

### REFERENCES

[Allard, B., Panariti, A., and Martin, J. G. (2018). Alveolar Macrophages in the Resolution of Inflammation, Tissue Repair, and Tolerance to Infection. Front. Immunol. 9, 1777] is incorporated by reference herein in its entirety.
[Barnes, B. J., Adrover, J. M., Baxter-Stoltzfus, A., Borczuk, A., Cools-Lartigue, J., Crawford, J. M., DaBler-Plenker, J., Guerci, P., Huynh, C., Knight, J. S., et al. (2020). Targeting potential drivers of COVID-19: Neutrophil extracellular traps. J. Exp. Med. 217, 1-7.] is incorporated by reference herein in its entirety.
[Blanco-Melo, D., Nilsson-Payant, B. E., Liu, W.-C., Uhl, S., Hoagland, D., Møller, R., Jordan, T. X., Oishi, K., Panis, M., David Sachs, et al. (2020). Imbalanced host response to SARS-CoV-2 drives development of COVID-19. Cell.] is incorporated by reference herein in its entirety.
[Catalina, M. D., Bachali, P., Geraci, N. S., Grammer, A. C., and Lipsky, P. E. (2019). Gene expression analysis delineates the potential roles of multiple interferons in systemic lupus erythematosus. Commun. Biol. 2.] is incorporated by reference herein in its entirety.
[Chafekar, A., and Fielding, B. C. (2018). MERS-CoV: Understanding the Latest Human Coronavirus Threat. Viruses 10.] is incorporated by reference herein in its entirety.
[Chen, G., Wu, D., Guo, W., Cao, Y., Huang, D., Wang, H., Wang, T., Zhang, X., Chen, H., Yu, H., et al. (2020a). Clinical and immunologic features in severe and moderate Coronavirus Disease 2019. J. Clin. Invest.] is incorporated by reference herein in its entirety.
[Chen, N., Zhou, M., Dong, X., Qu, J., Gong, F., Han, Y., Qiu, Y., Wang, J., Liu, Y., Wei, Y., et al. (2020b). Epidemiological and clinical characteristics of 99 cases of 2019 novel coronavirus pneumonia in Wuhan, China: a descriptive study. Lancet 395, 507-513.] is incorporated by reference herein in its entirety.
[Crayne, C. B., Albeituni, S., Nichols, K. E., and Cron, R. Q. (2019). The immunology of macrophage activation syndrome. Front. Immunol. 10, 1-11.] is incorporated by reference herein in its entirety.
[Cui, J., Li, F., and Shi, Z. L. (2019). Origin and evolution of pathogenic coronaviruses. Nat. Rev. Microbiol. 17, 181-192.] is incorporated by reference herein in its entirety.
[Culemann, S., Gruneboom, A., Nicolas-Avila, J. A., Weidner, D., Lammle, K. F., Rothe, T., Quintana, J. A., Kirchner, P., Krljanac, B., Eberhardt, M., et al. (2019). Locally renewing resident synovial macrophages provide a protective barrier for the joint. Nature 572, 670-675.
[Darwich, L., Coma, G., Peña, R., Bellido, R., Blanco, E. J. J., Este, J. A., Borras, F. E., Clotet, B., Ruiz, L., Rosell, A., et al. (2009). Secretion of interferon-γ by human macrophages demonstrated at the single-cell level after costimulation with interleukin (IL)-12 plus IL-18. Immunology 126, 386-393.] is incorporated by reference herein in its entirety.
[Drosten, C., Gunther, S., Preiser, W., van der Werf, S., Brodt, H.-R., Becker, S., Rabenau, H., Panning, M., Kolesnikova, L., Fouchier, R. A. M., et al. (2003). Identification of a novel coronavirus in patients with severe acute respiratory syndrome. N. Engl. J. Med. 348, 1967-1976.] is incorporated by reference herein in its entirety.
[Fung, T. S., and Liu, D. X. (2019). Human Coronavirus: Host-Pathogen Interaction. 529-560.] is incorporated by reference herein in its entirety.
[Gao, T., Hu, M., Zhang, X., Li, H., Zhu, L., Liu, H., Dong, Q., Zhang, Z., Wang, Z., Hu, Y., et al. (2020). Highly pathogenic coronavirus N protein aggravates lung injury by MASP-2-mediated complement over-activation. MedRxiv 2020.03.29.20041962.] is incorporated by reference herein in its entirety.
[Goritzka, M., Makris, S., Kausar, F., Durant, L. R., Pereira, C., Kumagai, Y., Culley, F. J., Mack, M., Akira, S., and Johansson, C. (2015). Alveolar macrophage-derived type I interferons orchestrate innate immunity to RSV through recruitment of antiviral monocytes. J. Exp. Med. 212, 699-714.] is incorporated by reference herein in its entirety.
[Greenberg, S. B. (2016). Update on Human Rhinovirus and Coronavirus Infections. Semin. Respir. Crit. Care Med. 37, 555-571.] is incorporated by reference herein in its entirety.
[He, Z., Zhao, C., Dong, Q., Zhuang, H., Song, S., Peng, G., and Dwyer, D. E. (2005). Effects of severe acute respiratory syndrome (SARS) coronavirus infection on peripheral blood lymphocytes and their subsets. Int. J. Infect. Dis. 9, 323-330.] is incorporated by reference herein in its entirety.
[Heron, M., Grutters, J. C., Ten Dam-Molenkamp, K. M., Hijdra, D., Van Heugten-Roeling, A., Claessen, A. M. E., Ruven, H. J. T., Van den Bosch, J. M. M., and Van Velzen-Blad, H. (2012). Bronchoalveolar lavage cell pattern from healthy human lung. Clin. Exp. Immunol. 167, 523-531.] is incorporated by reference herein in its entirety.
[Hoffmann, M., Kleine-Weber, H., Schroeder, S., Kruger, N., Herrler, T., Erichsen, S., Schiergens, T. S., Herrler, G., Wu, N. H., Nitsche, A., et al. (2020). SARS-CoV-2 Cell Entry Depends on ACE2 and TMPRSS2 and Is Blocked by a Clinically Proven Protease Inhibitor. Cell 181, 271-280.e8.] is incorporated by reference herein in its entirety.
[Huang, C., Wang, Y., Li, X., Ren, L., Zhao, J., Hu, Y., Zhang, L., Fan, G., Xu, J., Gu, X., et al. (2020). Clinical features of patients infected with 2019 novel coronavirus in Wuhan, China. Lancet 395, 497-506.] is incorporated by reference herein in its entirety.
[Iwasaki, A., and Yang, Y. (2020). The potential danger of suboptimal antibody responses in COVID-19. Nat. Rev. Immunol. 1-3.] is incorporated by reference herein in its entirety.
[Jing, C., Yue-Feng, X., Ma-Zhong, Z., and Li-Ming, Z. (2016). IL-33 Signaling in Lung Injury. Transl. Perioper. Pain Med. 3, 24-32.] is incorporated by reference herein in its entirety.
[Joshi, P. C., Applewhite, L., Mitchell, P. O., Fernainy, K., Roman, J., Eaton, D. C., and Guidot, D. M. (2006). GM-CSF receptor expression and signaling is decreased in lungs of ethanol-fed rats. Am. J. Physiol. Lung Cell. Mol. Physiol. 291, L1150-8.] is incorporated by reference herein in its entirety.
[Kelley, N., Jeltema, D., Duan, Y., and He, Y. (2019). The NLRP3 inflammasome: An overview of mechanisms of activation and regulation. Int. J. Mol. Sci. 20, 1-24.] is incorporated by reference herein in its entirety.
[Klok, F. A., Kruip, M. J. H. A., van der Meer, N. J. M., Arbous, M. S., Gommers, D. A. M. P. J., Kant, K. M., Kaptein, F. H. J., van Paassen, J., Stals, M. A. M., Huisman, M. V, et al. (2020). Incidence of thrombotic complications in critically ill ICU patients with COVID-19. Thromb. Res.] is incorporated by reference herein in its entirety.
[Lazear, H. M., Schoggins, J. W., and Diamond, M. S. (2019). Shared and Distinct Functions of Type I and Type III Interferons. Immunity 50, 907-923.] is incorporated by reference herein in its entirety.
[Liao, M., Liu, Y., Yuan, J., Wen, Y., Xu, G., Zhao, J., Chen, L., Li, J., Wang, X., Wang, F., et al. (2020). The landscape of lung bronchoalveolar immune cells in COVID-19 revealed by single-cell RNA sequencing. MedRxiv 2020.02.23.20026690.] is incorporated by reference herein in its entirety.
[Lindell, D. M., Lane, T. E., and Lukacs, N. W. (2008). CXCL10/CXCR3-mediated responses promote immunity to respiratory syncytial virus infection by augmenting dendritic cell and CD8 (+) T cell efficacy. Eur. J. Immunol. 38, 2168-2179.] is incorporated by reference herein in its entirety.
[Liu, L., Wei, Q., Lin, Q., Fang, J., Wang, H., Kwok, H., Tang, H., Nishiura, K., Peng, J., Tan, Z., et al. (2019). Anti-spike IgG causes severe acute lung injury by skewing macrophage responses during acute SARS-CoV infection. JCI Insight 4, 1-19.] is incorporated by reference herein in its entirety.
[Lovren, F., Pan, Y., Quan, A., Teoh, H., Wang, G., Shukla, P. C., Levitt, K. S., Oudit, G. Y., A1-Omran, M., Stewart, D. J., et al. (2008). Angiotensin converting enzyme-2 confers endothelial protection and attenuates atherosclerosis. Am. J. Physiol.—Hear. Circ. Physiol. 295, 1377-1384.] is incorporated by reference herein in its entirety.
[Martinez, F. O., Gordon, S., Locati, M., and Mantovani, A. (2006). Transcriptional profiling of the human monocyte-to-macrophage differentiation and polarization: new molecules and patterns of gene expression. J. Immunol. 177, 7303-7311.] is incorporated by reference herein in its entirety.
[McGonagle, D., Sharif, K., O'Regan, A., and Bridgewood, C. (2020). The Role of Cytokines including Interleukin-6 in COVID-19 induced Pneumonia and Macrophage Activation Syndrome-Like Disease. Autoimmun. Rev. 102537.] is incorporated by reference herein in its entirety.
[Mehta, P., McAuley, D. F., Brown, M., Sanchez, E., Tattersall, R. S., and Manson, J. J. (2020). COVID-19: consider cytokine storm syndromes and immunosuppression. Lancet (London, England) 395, 1033-1034.] is incorporated by reference herein in its entirety.
[Middeldorp, S., Coppens, M., van Haaps, T. F., Foppen, M., Vlaar, A. P., Muller, M. C. A., Bouman, C. C. S., Beenen, L. F. M., Kootte, R. S., Heijmans, J., et al. (2020). Incidence of venous thromboembolism in hospitalized patients with COVID-19. J. Thromb. Haemost.] is incorporated by reference herein in its entirety.
[Newton, A. H., Cardani, A., and Braciale, T. J. (2016). The host immune response in respiratory virus infection: balancing virus clearance and immunopathology. Semin. Immunopathol. 38, 471-482.] is incorporated by reference herein in its entirety.
[Pattterson, B., Corley, M., Pang, A., and Wu, H. Disruption of the CCL5/RANTES-CCR5 Pathway Restores Immune Homeostasis and Reduces Plasma Viral Load in Critical COVID-19 CURRENT STATUS: POSTED. 1-20.] is incorporated by reference herein in its entirety.
[Pedersen, S. F., and Ho, Y.-C. (2020). SARS-CoV-2: a storm is raging. J. Clin. Invest. 130, 2202-2205.] is incorporated by reference herein in its entirety.
[Qin, C., Zhou, L., Hu, Z., Zhang, S., Yang, S., Tao, Y., Xie, C., Ma, K., Shang, K., Wang, W., et al. (2020). Dysregulation of immune response in patients with COVID-19 in Wuhan, China. Clin. Infect. Dis. 2019, 4-10.] is incorporated by reference herein in its entirety.
[Reyfman, P. A., Walter, J. M., Joshi, N., Anekalla, K. R., McQuattie-Pimentel, A. C., Chiu, S., Fernandez, R., Akbarpour, M., Chen, C.-I., Ren, Z., et al. (2019). Single-Cell Transcriptomic Analysis of Human Lung Provides Insights into the Pathobiology of Pulmonary Fibrosis. Am. J. Respir. Crit. Care Med. 199, 1517-1536.] is incorporated by reference herein in its entirety.
[Schmitz, N., Kurrer, M., Bachmann, M. F., and Kopf, M. (2005). Interleukin-1 Is Responsible for Acute Lung Immunopathology but Increases Survival of Respiratory Influenza Virus Infection. J. Virol. 79, 6441-6448.] is incorporated by reference herein in its entirety.
[Sungnak, W., Huang, N., Bécavin, C., Berg, M., Queen, R., Litvinukova, M., Talavera-Lopez, C., Maatz, H., Reichart, D., Sampaziotis, F., et al. (2020). SARS-CoV-2 entry factors are highly expressed in nasal epithelial cells together with innate immune genes. Nat. Med. 1-7.] is incorporated by reference herein in its entirety.
[Suzuki, T., Arumugam, P., Sakagami, T., Lachmann, N., Chalk, C., Sallese, A., Abe, S., Trapnell, C., Carey, B., Moritz, T., et al. (2014). Pulmonary macrophage transplantation therapy. Nature 514, 450-454.] is incorporated by reference herein in its entirety.
[Tang, N. L. S., Chan, P. K. S., Wong, C. K., To, K. F., Wu, A. K. L., Sung, Y. M., Hui, D. S. C., Sung, J. J. Y., and Lam, C. W. K. (2005). Early enhanced expression of interferon-inducible protein-10 (CXCL-10) and other chemokines predicts adverse outcome in severe acute respiratory syndrome. Clin. Chem. 51, 2333-2340.] is incorporated by reference herein in its entirety.
[Tay, M. Z., Poh, C. M., Rénia, L., Macary, P. A., and Ng, L. F. P. The trinity of COVID-19: immunity, inflammation and intervention 1,2.] is incorporated by reference herein in its entirety.
[Teijaro, J. R. (2015). The role of cytokine responses during influenza virus pathogenesis and potential therapeutic options. Curr. Top. Microbiol. Immunol. 386, 3-22.] is incorporated by reference herein in its entirety.
[Thierry, A. R., and Roch, B. (2020). NETs by-products and extracellular DNA may play a key role in COVID-19 pathogenesis: incidence on patient monitoring and therapy. 1-21.] is incorporated by reference herein in its entirety.
[Tipping, P. G., Campbell, D. A., Boyce, N. W., and Holdsworth, S. R. (1988). Alveolar macrophage procoagulant activity is increased in acute hyperoxic lung injury. Am. J. Pathol. 131, 206-212.] is incorporated by reference herein in its entirety.
[Trouillet-Assant, S., Viel, S., Gaymard, A., Pons, S., Richard, J.-C., Perret, M., Villard, M., Brengel-Pesce, K., Lina, B., Mezidi, M., et al. (2020). Type I IFN immunoprofiling in COVID-19 patients. J. Allergy Clin. Immunol.] is incorporated by reference herein in its entirety.
[Viola, A., Munari, F., Sanchez-Rodriguez, R., Scolaro, T., and Castegna, A. (2019). The metabolic signature of macrophage responses. Front. Immunol. 10, 1-16.] is incorporated by reference herein in its entirety.
[Voiriot, G., Razazi, K., Amsellem, V., Tran Van Nhieu, J., Abid, S., Adnot, S., Mekontso Dessap, A., and Maitre, B. (2017). Interleukin-6 displays lung anti-inflammatory properties and exerts protective hemodynamic effects in a double-hit murine acute lung injury. Respir. Res. 18, 64.] is incorporated by reference herein in its entirety.
[Wang, S.-Z., Bao, Y.-X., Rosenberger, C. L., Tesfaigzi, Y., Stark, J. M., and Harrod, K. S. (2004). IL-12p40 and IL-18 modulate inflammatory and immune responses to respiratory syncytial virus infection. J. Immunol. 173, 4040-4049.] is incorporated by reference herein in its entirety.
[Wang, X., Guo, X., Xin, Q., Pan, Y., Li, J., Chu, Y., Feng, Y., and Wang, Q. (2020). Neutralizing Antibodies Responses to SARS-CoV-2 in COVID-19 Inpatients and Convalescent Patients. MedRxiv 2020.04.15.20065623.] is incorporated by reference herein in its entirety.
[Wei, L., Ming, S., Zou, B., Wu, Y., Hong, Z., Li, Z., Zheng, X., Huang, M., Luo, L., Liang, J., et al. (2020). Viral Invasion and Type I Interferon Response Characterize the Immunophenotypes During Covid-19 Infection. SSRN Electron. J.] is incorporated by reference herein in its entirety.
[Wen, W., Su, W., Tang, H., Le, W., Zhang, X., and Zheng, Y. (2020). Immune Cell Profiling of COVID-19 Patients in the recovery stage by Single-cell sequencing.] is incorporated by reference herein in its entirety.
[Wichmann, D., Sperhake, J.-P., Lütgehetmann, M., Steurer, S., Edler, C., Heinemann, A., Heinrich, F., Mushumba, H., Kniep, I., Schröder, A. S., et al. (2020). Autopsy Findings and Venous Thromboembolism in Patients With COVID-19: A Prospective Cohort Study. Ann. Intern. Med.] is incorporated by reference herein in its entirety.
[Wu, F., Wang, A., Liu, M., Wang, Q., Chen, J., Xia, S., Ling, Y., Zhang, Y., Xun, J., Lu, L., et al. (2020). Neutralizing antibody responses to SARS-CoV-2 in a COVID-19 recovered patient cohort and their implications. MedRxiv 2020.03.30.20047365.] is incorporated by reference herein in its entirety.
[Xiong, Y., Liu, Y., Cao, L., Wang, D., Guo, M., Jiang, A., Guo, D., Hu, W., Yang, J., Tang, Z., et al. (2020). Transcriptomic characteristics of bronchoalveolar lavage fluid and peripheral blood mononuclear cells in COVID-19 patients. Emerg. Microbes Infect. 9, 761-770.] is incorporated by reference herein in its entirety.
[Xu, Z., Shi, L., Wang, Y., Zhang, J., Huang, L., Zhang, C., Liu, S., Zhao, P., Liu, H., Zhu, L., et al. (2020). Pathological findings of COVID-19 associated with acute respiratory distress syndrome. Lancet Respir. Med. 8, 420-422.] is incorporated by reference herein in its entirety.
[Zhang, B., Zhou, X., Qiu, Y., Feng, F., Feng, J., Jia, Y., Zhu, H., Hu, K., Liu, J., Liu, Z., et al. (2020). Clinical characteristics of 82 death cases with COVID-19. MedRxiv 2020.02.26.20028191.] is incorporated by reference herein in its entirety.

Table of 33 Modules

| geneSymbol | geneEntrezID | GeneSet |
|---|---|---|
| CD160 | 11126 | Anergic/Activated T cells |
| CD244 | 51744 | Anergic/Activated T cells |
| CTLA4 | 1493 | Anergic/Activated T cells |
| HAVCR2 | 84868 | Anergic/Activated T cells |
| ICOS | 29851 | Anergic/Activated T cells |
| KLRG1 | 10219 | Anergic/Activated T cells |
| LAG3 | 3902 | Anergic/Activated T cells |
| PDCD1 | 5133 | Anergic/Activated T cells |
| IL1RN | 3557 | Anti inflammation |
| TNFAIP3 | 7128 | Anti inflammation |
| SOCS3 | 9021 | Anti inflammation |
| IGHD | 3495 | B cells |
| SH3BP5 | 9467 | B cells |
| BANK1 | 55024 | B cells |
| BLK | 640 | B cells |
| BLNK | 29760 | B cells |
| CD19 | 930 | B cells |
| CD22 | 933 | B cells |
| CD79A | 973 | B cells |
| CD79B | 974 | B cells |
| DAPP1 | 27071 | B cells |
| FCRL1 | 115350 | B cells |
| FCRL2 | 79368 | B cells |
| FCRL3 | 115352 | B cells |
| FCRLA | 84824 | B cells |
| GON4L | 54856 | B cells |
| GPR183 | 1880 | B cells |
| IGHM | 3507 | B cells |
| KLHL6 | 89857 | B cells |
| MS4A1 | 931 | B cells |
| PAX5 | 5079 | B cells |
| PLCL2 | 23228 | B cells |
| VPREB1 | 7441 | B cells |
| ZNF318 | 24149 | B cells |
| CD8A | 925 | CD8T-NK-NKT |
| CD8B | 926 | CD8T-NK-NKT |
| GZMB | 3002 | CD8T-NK-NKT |
| NKTR | 4820 | CD8T-NK-NKT |
| CD2 | 914 | CD8T-NK-NKT |
| CD7 | 924 | CD8T-NK-NKT |
| CRTAM | 56253 | CD8T-NK-NKT |
| GNLY | 10578 | CD8T-NK-NKT |
| GZMA | 3001 | CD8T-NK-NKT |
| GZMK | 3003 | CD8T-NK-NKT |
| GZMM | 3004 | CD8T-NK-NKT |
| HCST | 10870 | CD8T-NK-NKT |
| KIR2DL3 | 3804 | CD8T-NK-NKT |
| KIR3DL1 | 3811 | CD8T-NK-NKT |
| KIR3DL2 | 3812 | CD8T-NK-NKT |
| KLRB1 | 3820 | CD8T-NK-NKT |
| KLRC3 | 3823 | CD8T-NK-NKT |
| KLRC4 | 8302 | CD8T-NK-NKT |
| KLRD1 | 3824 | CD8T-NK-NKT |
| NKG7 | 4818 | CD8T-NK-NKT |
| RASAL3 | 64926 | CD8T-NK-NKT |
| TIA1 | 7072 | CD8T-NK-NKT |
| TXK | 7294 | CD8T-NK-NKT |
| BRCA1 | 672 | Cell Cycle |
| ASPM | 259266 | Cell Cycle |
| AURKA | 6790 | Cell Cycle |
| AURKB | 9212 | Cell Cycle |
| CCNB1 | 891 | Cell Cycle |
| CCNB2 | 9133 | Cell Cycle |
| CCNE1 | 898 | Cell Cycle |
| CDC20 | 991 | Cell Cycle |
| CENPM | 79019 | Cell Cycle |
| CEP55 | 55165 | Cell Cycle |
| E2F3 | 1871 | Cell Cycle |
| GINS2 | 51659 | Cell Cycle |
| MCM10 | 55388 | Cell Cycle |
| MCM2 | 4171 | Cell Cycle |
| MKI67 | 4288 | Cell Cycle |
| NCAPG | 64151 | Cell Cycle |
| NDC80 | 10403 | Cell Cycle |
| PTTG1 | 9232 | Cell Cycle |
| TYMS | 7298 | Cell Cycle |
| GZMB | 3002 | Cytotoxic, Activated T cells |
| TAGAP | 117289 | Cytotoxic, Activated T cells |
| CD69 | 969 | Cytotoxic, Activated T cells |
| EOMES | 8320 | Cytotoxic, Activated T cells |
| GZMH | 2999 | Cytotoxic, Activated T cells |
| IFNG | 3458 | Cytotoxic, Activated T cells |
| IL2RB | 3560 | Cytotoxic, Activated T cells |
| PRF1 | 5551 | Cytotoxic, Activated T cells |
| SGK1 | 6446 | Cytotoxic, Activated T cells |
| TBX21 | 30009 | Cytotoxic, Activated T cells |
| TFRC | 7037 | Cytotoxic, Activated T cells |
| ZNF683 | 257101 | Cytotoxic, Activated T cells |
| CLEC12A | 160364 | Dendritic |
| CLEC10A | 10462 | Dendritic |
| CLEC9A | 283420 | Dendritic |
| CSF1R | 1436 | Dendritic |
| IGIP | 492311 | Dendritic |
| LILRA4 | 23547 | Dendritic |
| LY75 | 4065 | Dendritic |
| XCR1 | 2829 | Dendritic |
| EPO | 2056 | Erythrocytes |
| GFI1B | 8328 | Erythrocytes |
| GYPA | 2993 | Erythrocytes |
| GYPB | 2994 | Erythrocytes |
| GYPE | 2996 | Erythrocytes |
| ICAM4 | 3386 | Erythrocytes |
| NFE2 | 4778 | Erythrocytes |
| SLC4A1 | 6521 | Erythrocytes |
| TRIM10 | 10107 | Erythrocytes |
| TSPO2 | 222642 | Erythrocytes |
| ZNF367 | 195828 | Erythrocytes |
| CXCR2 | 3579 | Granulocyte |
| CD177 | 57126 | Granulocyte |
| CLC | 1178 | Granulocyte |
| CTSS | 1520 | Granulocyte |
| DEFA1 | 1667 | Granulocyte |
| FUT7 | 2529 | Granulocyte |
| LTB4R | 1241 | Granulocyte |
| MMP25 | 64386 | Granulocyte |
| OSM | 5008 | Granulocyte |
| RETN | 56729 | Granulocyte |
| GBP1 | 2633 | IFN |
| IFI44 | 10561 | IFN |
| MX1 | 4599 | IFN |
| NAMPT | 10135 | IFN |
| NKTR | 4820 | IFN |
| NR3C1 | 2908 | IFN |
| OAS3 | 4940 | IFN |
| PATJ | 10207 | IFN |
| TAP2 | 6891 | IFN |
| TNFRSF11A | 8792 | IFN |
| FASLG | 356 | IFN |
| WT1 | 7490 | IFN |
| EIF2AK2 | 5610 | IFN |
| GBP2 | 2634 | IFN |
| GBP4 | 115361 | IFN |
| HERC5 | 51191 | IFN |
| HERC6 | 55008 | IFN |
| IFI27 | 3429 | IFN |
| IFI30 | 10437 | IFN |
| IFI35 | 3430 | IFN |
| IFI44L | 10964 | IFN |
| IFI6 | 2537 | IFN |
| IFIT1 | 3434 | IFN |
| IFIT2 | 3433 | IFN |
| IFIT3 | 3437 | IFN |
| IFIT5 | 24138 | IFN |
| IFITM1 | 8519 | IFN |
| IFITM2 | 10581 | IFN |
| IFITM3 | 10410 | IFN |
| ISG15 | 9636 | IFN |
| ISG20 | 3669 | IFN |
| MX2 | 4600 | IFN |
| NFE2L3 | 9603 | IFN |
| NMI | 9111 | IFN |
| NUB1 | 51667 | IFN |
| NUPR1 | 26471 | IFN |
| OAS1 | 4938 | IFN |
| OAS2 | 4939 | IFN |
| OASL | 8638 | IFN |
| PDGFB | 5155 | IFN |
| PDGFRL | 5157 | IFN |
| PKD2 | 5311 | IFN |
| PLSCR1 | 5359 | IFN |
| PMAIP1 | 5366 | IFN |
| PML | 5371 | IFN |
| PRKRA | 8575 | IFN |
| PSMB9 | 5698 | IFN |
| PTCH1 | 5727 | IFN |
| RBCK1 | 10616 | IFN |
| RGS1 | 5996 | IFN |
| RGS6 | 9628 | IFN |
| RSAD2 | 91543 | IFN |
| RTP4 | 64108 | IFN |
| SAMD9 | 54809 | IFN |
| SAMD9L | 219285 | IFN |
| SAT1 | 6303 | IFN |
| SCARB2 | 950 | IFN |
| SERPING1 | 710 | IFN |
| SIT1 | 27240 | IFN |
| SOCS1 | 8651 | IFN |
| SP100 | 6672 | IFN |
| SP110 | 3431 | IFN |
| SP140 | 11262 | IFN |
| SPIB | 6689 | IFN |
| ST3GAL5 | 8869 | IFN |
| STAP1 | 26228 | IFN |
| STAT1 | 6772 | IFN |
| STAT2 | 6773 | IFN |
| STX11 | 8676 | IFN |
| SUPT3H | 8464 | IFN |
| TAP1 | 6890 | IFN |
| TARBP1 | 6894 | IFN |
| TCN2 | 6948 | IFN |
| TFDP2 | 7029 | IFN |
| TGM1 | 7051 | IFN |
| TLR3 | 7098 | IFN |
| TLR7 | 51284 | IFN |
| TNFSF10 | 8743 | IFN |
| TNK2 | 10188 | IFN |
| TOR1B | 27348 | IFN |
| TRA2B | 6434 | IFN |
| TRD | 6964 | IFN |
| TRIM21 | 6737 | IFN |
| TRIM22 | 10346 | IFN |
| TRIM34 | 53840 | IFN |
| TRIM38 | 10475 | IFN |
| UBA7 | 7318 | IFN |
| UBE2L6 | 9246 | IFN |
| UBE2S | 27338 | IFN |
| UNC93B1 | 81622 | IFN |
| USP18 | 11274 | IFN |
| WARS | 7453 | IFN |
| XAF1 | 54739 | IFN |
| IGHG1 | 3500 | IG CHAINS |
| IGHV2-5 | 28457 | IG CHAINS |
| IGHV3-20 | 28445 | IG CHAINS |
| IGHV3-23 | 28442 | IG CHAINS |
| IGHV4-28 | 28400 | IG CHAINS |
| IGHV4-34 | 28395 | IG CHAINS |
| IGKC | 3514 | IG CHAINS |
| IGLV1-40 | 28825 | IG CHAINS |
| IGLV3-1 | 28809 | IG CHAINS |
| IGLV3-19 | 28797 | IG CHAINS |
| IGLV3-25 | 28793 | IG CHAINS |
| IGLV4-3 | 28786 | IG CHAINS |
| IGLV4-60 | 28785 | IG CHAINS |
| IGLV5-45 | 28781 | IG CHAINS |
| IGLV6-57 | 28778 | IG CHAINS |
| IGLVI-70 | 28763 | IG CHAINS |
| IGHA1 | 3493 | IG CHAINS |
| IGHA2 | 3494 | IG CHAINS |
| IGHD2-15 | 28503 | IG CHAINS |
| IGHD2-2 | 28505 | IG CHAINS |
| IGHD2-21 | 28502 | IG CHAINS |
| IGHD3-10 | 28499 | IG CHAINS |
| IGHD3-16 | 28498 | IG CHAINS |
| IGHD3-3 | 28501 | IG CHAINS |
| IGHD3-9 | 28500 | IG CHAINS |
| IGHG2 | 3501 | IG CHAINS |
| IGHG3 | 3502 | IG CHAINS |
| IGHG4 | 3503 | IG CHAINS |
| IGHJ1 | 28483 | IG CHAINS |
| IGHJ2 | 28481 | IG CHAINS |
| IGHJ3 | 28479 | IG CHAINS |
| IGHJ4 | 28477 | IG CHAINS |
| IGHJ5 | 28476 | IG CHAINS |
| IGHJ6 | 28475 | IG CHAINS |
| IGHV1-18 | 28468 | IG CHAINS |
| IGHV1-2 | 28474 | IG CHAINS |
| IGHV1-24 | 28467 | IG CHAINS |
| IGHV1-3 | 28473 | IG CHAINS |
| IGHV1-45 | 28466 | IG CHAINS |
| IGHV1-46 | 28465 | IG CHAINS |
| IGHV1-58 | 28464 | IG CHAINS |
| IGHV1-69-2 | 28458 | IG CHAINS |
| IGHV2-26 | 28455 | IG CHAINS |
| IGHV2-70 | 28454 | IG CHAINS |
| IGHV3-13 | 28449 | IG CHAINS |
| IGHV3-15 | 28448 | IG CHAINS |
| IGHV3-21 | 28444 | IG CHAINS |
| IGHV3-33 | 28434 | IG CHAINS |
| IGHV3-43 | 28426 | IG CHAINS |
| IGHV3-48 | 28424 | IG CHAINS |
| IGHV3-49 | 28423 | IG CHAINS |
| IGHV3-53 | 28420 | IG CHAINS |
| IGHV3-62 | 28416 | IG CHAINS |
| IGHV3-64 | 28414 | IG CHAINS |
| IGHV3-7 | 28452 | IG CHAINS |
| IGHV3-72 | 28410 | IG CHAINS |
| IGHV3-73 | 28409 | IG CHAINS |
| IGHV3-74 | 28408 | IG CHAINS |
| IGHV4-30-2 | 28398 | IG CHAINS |
| IGHV4-39 | 28394 | IG CHAINS |
| IGHV4-59 | 28392 | IG CHAINS |
| IGHV4-61 | 28391 | IG CHAINS |
| IGHV5-51 | 28388 | IG CHAINS |
| IGHV6-1 | 28385 | IG CHAINS |
| IGHV7-81 | 28378 | IG CHAINS |
| IGKJ1 | 28950 | IG CHAINS |
| IGKJ2 | 28949 | IG CHAINS |
| IGKJ3 | 28948 | IG CHAINS |
| IGKJ4 | 28947 | IG CHAINS |
| IGKJ5 | 28946 | IG CHAINS |
| IGKV1-16 | 28938 | IG CHAINS |
| IGKV1-17 | 28937 | IG CHAINS |
| IGKV1-27 | 28935 | IG CHAINS |
| IGKV1-5 | 28299 | IG CHAINS |
| IGKV1-6 | 28943 | IG CHAINS |
| IGKV1-9 | 28941 | IG CHAINS |
| IGKV1D-16 | 28901 | IG CHAINS |
| IGKV1D-17 | 28900 | IG CHAINS |
| IGKV1D-43 | 28891 | IG CHAINS |
| IGKV1D-8 | 28904 | IG CHAINS |
| IGKV2-24 | 28923 | IG CHAINS |
| IGKV2D-26 | 28884 | IG CHAINS |
| IGKV2D-29 | 28882 | IG CHAINS |
| IGKV2D-30 | 28881 | IG CHAINS |
| IGKV3-20 | 28912 | IG CHAINS |
| IGKV3D-20 | 28874 | IG CHAINS |
| IGKV3D-7 | 28877 | IG CHAINS |
| IGKV4-1 | 28908 | IG CHAINS |
| IGKV5-2 | 28907 | IG CHAINS |
| IGLC2 | 3538 | IG CHAINS |
| IGLC7 | 28834 | IG CHAINS |
| IGLJ6 | 28828 | IG CHAINS |
| IGLV10-54 | 28772 | IG CHAINS |
| IGLV1-36 | 28826 | IG CHAINS |
| IGLV1-47 | 28822 | IG CHAINS |
| IGLV2-11 | 28816 | IG CHAINS |
| IGLV2-18 | 28814 | IG CHAINS |
| IGLV2-23 | 28813 | IG CHAINS |
| IGLV2-33 | 28811 | IG CHAINS |
| IGLV2-8 | 28817 | IG CHAINS |
| IGLV3-10 | 28803 | IG CHAINS |
| IGLV3-12 | 28802 | IG CHAINS |
| IGLV3-16 | 28799 | IG CHAINS |
| IGLV3-21 | 28796 | IG CHAINS |
| IGLV3-27 | 28791 | IG CHAINS |
| IGLV3-32 | 28787 | IG CHAINS |
| IGLV4-69 | 28784 | IG CHAINS |
| IGLV5-37 | 28783 | IG CHAINS |
| IGLV7-43 | 28776 | IG CHAINS |
| IGLV8-61 | 28774 | IG CHAINS |
| IGLV9-49 | 28773 | IG CHAINS |
| IL18 | 3606 | IL1 cytokines |
| IL1B | 3553 | IL1 cytokines |
| CASP1 | 834 | Inflammasome |
| AIM2 | 9447 | Inflammasome |
| CASP5 | 838 | Inflammasome |
| CTSB | 1508 | Inflammasome |
| GSDMB | 55876 | Inflammasome |
| GSDMD | 79792 | Inflammasome |
| NAIP | 4671 | Inflammasome |
| NEK7 | 140609 | Inflammasome |
| NLRC4 | 58484 | Inflammasome |
| NLRP1 | 22861 | Inflammasome |
| NLRP3 | 114548 | Inflammasome |
| NOD2 | 64127 | Inflammasome |
| P2RX7 | 5027 | Inflammasome |
| PANX1 | 24145 | Inflammasome |
| PYCARD | 29108 | Inflammasome |
| RIPK1 | 8737 | Inflammasome |
| AZU1 | 566 | LDG |
| CAMP | 820 | LDG |
| CEACAM6 | 4680 | LDG |
| CEACAM8 | 1088 | LDG |
| CTSG | 1511 | LDG |
| DEFA4 | 1669 | LDG |
| ELANE | 1991 | LDG |
| LCN2 | 3934 | LDG |
| LTF | 4057 | LDG |
| MPO | 4353 | LDG |
| OLFM4 | 10562 | LDG |
| RNASE3 | 6037 | LDG |
| HLA-DMA | 3108 | MHC II |
| HLA-DMB | 3109 | MHC II |
| HLA-DPA1 | 3113 | MHC II |
| HLA-DPB1 | 3115 | MHC II |
| HLA-DPB2 | 3116 | MHC II |
| HLA-DQA1 | 3117 | MHC II |
| HLA-DQA2 | 3118 | MHC II |
| HLA-DQB1 | 3119 | MHC II |
| HLA-DQB2 | 3120 | MHC II |
| HLA-DRA | 3122 | MHC II |
| HLA-DRB1 | 3123 | MHC II |
| HLA-DRB3 | 3125 | MHC II |
| HLA-DRB4 | 3126 | MHC II |
| HLA-DRB5 | 3127 | MHC II |
| HLA-DRB6 | 3128 | MHC II |
| C1QA | 712 | Mono Secreted |
| C1QB | 713 | Mono Secreted |
| C1QC | 714 | Mono Secreted |
| C1RL | 51279 | Mono Secreted |
| CCL2 | 6347 | Mono Secreted |
| CCL8 | 6355 | Mono Secreted |
| CXCL1 | 2919 | Mono Secreted |
| CXCL2 | 2920 | Mono Secreted |
| GRN | 2896 | Mono Secreted |
| IK | 3550 | Mono Secreted |
| IL18RAP | 8807 | Mono Secreted |
| IL1B | 3553 | Mono Secreted |
| IL1RN | 3557 | Mono Secreted |
| S100A8 | 6279 | Mono Secreted |
| THBD | 7056 | Mono Secreted |
| TNF | 7124 | Mono Secreted |
| CXCL10 | 3627 | Mono Secreted |
| CD14 | 929 | Monocyte Cell Surface |
| CD300C | 10871 | Monocyte Cell Surface |
| CD33 | 945 | Monocyte Cell Surface |
| CD68 | 968 | Monocyte Cell Surface |
| CLEC12A | 160364 | Monocyte Cell Surface |
| CLEC4D | 338339 | Monocyte Cell Surface |
| CLEC4E | 26253 | Monocyte Cell Surface |
| FCGR1A | 2209 | Monocyte Cell Surface |
| FCGR1B | 2210 | Monocyte Cell Surface |
| FCGR3B | 2215 | Monocyte Cell Surface |
| LILRA5 | 353514 | Monocyte Cell Surface |
| LILRA6 | 79168 | Monocyte Cell Surface |
| LILRB2 | 10288 | Monocyte Cell Surface |
| LILRB3 | 11025 | Monocyte Cell Surface |
| OSCAR | 126014 | Monocyte Cell Surface |
| SEMA4A | 64218 | Monocyte Cell Surface |
| SIGLEC1 | 6614 | Monocyte Cell Surface |
| C1QA | 712 | Monocytes |
| C1QB | 713 | Monocytes |
| C1QC | 714 | Monocytes |
| C1RL | 51279 | Monocytes |
| CCL2 | 6347 | Monocytes |
| CCL8 | 6355 | Monocytes |
| CD14 | 929 | Monocytes |
| CD300C | 10871 | Monocytes |
| CD33 | 945 | Monocytes |
| CD68 | 968 | Monocytes |
| CLEC12A | 160364 | Monocytes |
| CLEC4D | 338339 | Monocytes |
| CLEC4E | 26253 | Monocytes |
| CXCL1 | 2919 | Monocytes |
| CXCL2 | 2920 | Monocytes |
| CXCR2 | 3579 | Monocytes |
| FCGR1A | 2209 | Monocytes |
| FCGR1B | 2210 | Monocytes |
| FCGR1B | 2215 | Monocytes |
| GRN | 2896 | Monocytes |
| IK | 3550 | Monocytes |
| IL18RAP | 8807 | Monocytes |
| IL1B | 3553 | Monocytes |
| IL1RN | 3557 | Monocytes |
| LILRA5 | 353514 | Monocytes |
| LILRA6 | 79168 | Monocytes |
| LILRB2 | 10288 | Monocytes |
| LILRB3 | 11025 | Monocytes |
| OSCAR | 126014 | Monocytes |
| S100A8 | 6279 | Monocytes |
| SEMA4A | 64218 | Monocytes |
| SIGLEC1 | 6614 | Monocytes |
| THBD | 7056 | Monocytes |
| BST1 | 683 | Monocytes |
| C1R | 715 | Monocytes |
| CD163 | 9332 | Monocytes |
| CSF2 | 1437 | Monocytes |
| FUT4 | 2526 | Monocytes |
| MNDA | 4332 | Monocytes |
| MRC1 | 4360 | Monocytes |
| KLRF1 | 51348 | NK |
| NCAM1 | 4684 | NK |
| NCR1 | 9437 | NK |
| NCR3 | 259197 | NK |
| SH2D1B | 117157 | NK |
| CLEC4C | 170482 | pDC |
| NRP1 | 8829 | pDC |
| IGHV4-28 | 28400 | Plasma Cells |
| IGHV4-34 | 28395 | Plasma Cells |
| IGHD | 3495 | Plasma Cells |
| IGHG1 | 3500 | Plasma Cells |
| IGHV2-5 | 28457 | Plasma Cells |
| IGHV3-20 | 28445 | Plasma Cells |
| IGHV3-23 | 28442 | Plasma Cells |
| IGKC | 3514 | Plasma Cells |
| IGLV1-40 | 28825 | Plasma Cells |
| IGLV3-1 | 28809 | Plasma Cells |
| IGLV3-19 | 28797 | Plasma Cells |
| IGLV3-25 | 28793 | Plasma Cells |
| IGLV4-3 | 28786 | Plasma Cells |
| IGLV4-60 | 28785 | Plasma Cells |
| IGLV5-45 | 28781 | Plasma Cells |
| IGLV6-57 | 28778 | Plasma Cells |
| IGLVI-70 | 28763 | Plasma Cells |
| C19orf10 | 56005 | Plasma Cells |
| IGH | 3492 | Plasma Cells |
| IGHMBP2 | 3508 | Plasma Cells |
| IGHV4-31 | 28396 | Plasma Cells |
| IGK | 50802 | Plasma Cells |
| IGL | 3535 | Plasma Cells |
| IGLJ3 | 28831 | Plasma Cells |
| IGLL1 | 3543 | Plasma Cells |
| IGLV@ | 3546 | Plasma Cells |
| IGLV1-44 | 28823 | Plasma Cells |
| IGLV2-14 | 28815 | Plasma Cells |
| IGLV2-5 | 28818 | Plasma Cells |
| MZB1 | 51237 | Plasma Cells |
| PRDM1 | 639 | Plasma Cells |
| SDC1 | 6382 | Plasma Cells |
| THEMIS2 | 9473 | Plasma Cells |
| TNFRSF17 | 608 | Plasma Cells |
| GP1BA | 2811 | Platelets |
| GP5 | 2814 | Platelets |
| GP6 | 51206 | Platelets |
| GP9 | 2815 | Platelets |
| LY6G6D | 58530 | Platelets |
| MMRN1 | 22915 | Platelets |
| PEAR1 | 375033 | Platelets |
| PF4 | 5196 | Platelets |
| PF4V1 | 5197 | Platelets |
| PPBP | 5473 | Platelets |
| SLC35D3 | 340146 | Platelets |
| RNU4ATAC | 100151683 | SNOR LOW Down |
| SNORA11 | 677799 | SNOR LOW Down |
| SNORA16A | 692073 | SNOR LOW Down |
| SNORA23 | 677808 | SNOR LOW Down |
| SNORA38B | 100124536 | SNOR LOW Down |
| SNORA73A | 6080 | SNOR LOW Down |
| SNORA73B | 26768 | SNOR LOW Down |
| FCGR1A | 2209 | SNOR LOW Up |
| CEACAM1 | 634 | SNOR LOW Up |
| LGALS1 | 3956 | SNOR LOW Up |
| SNORD24 | 26820 | SNOR LOW Up |
| SNORD44 | 26806 | SNOR LOW Up |
| SNORD47 | 26802 | SNOR LOW Up |
| SNORD80 | 26774 | SNOR LOW Up |
| CD8A | 925 | T Cells |
| CD8B | 926 | T Cells |
| TRDC | 28526 | T Cells |
| CCR3 | 1232 | T Cells |
| CD226 | 10666 | T Cells |
| CD247 | 919 | T Cells |
| CD28 | 940 | T Cells |
| CD3D | 915 | T Cells |
| CD3E | 916 | T Cells |
| CD3G | 917 | T Cells |
| CD4 | 920 | T Cells |
| CD5 | 921 | T Cells |
| ETS1 | 2113 | T Cells |
| GATA3 | 2625 | T Cells |
| GRAP2 | 9402 | T Cells |
| LEF1 | 51176 | T Cells |
| SH2D1A | 4068 | T Cells |
| TRAC | 28755 | T Cells |
| TRBC1 | 28639 | T Cells |
| FOXP3 | 50943 | T reg |
| IKZF2 | 22807 | T reg |
| FAS LG | 356 | Tactivated |
| TAGAP | 117289 | Tactivated |
| CD40LG | 959 | Tactivated |
| CREM | 1390 | Tactivated |
| IL17A | 3605 | Tactivated |
| IL17F | 112744 | Tactivated |
| IL23R | 149233 | Tactivated |
| JAKMIP1 | 152789 | Tactivated |
| KCNA3 | 3738 | Tactivated |
| KCNN4 | 3783 | Tactivated |
| P2RX5 | 5026 | Tactivated |
| PRKCQ | 5588 | Tactivated |
| RELT | 84957 | Tactivated |
| RNF125 | 54941 | Tactivated |
| SATB1 | 6304 | Tactivated |
| TNFRSF4 | 7293 | Tactivated |
| TNFRSF9 | 3604 | Tactivated |
| TRAV10 | 28676 | TCRA |
| TRAV1-1 | 28693 | TCRA |
| TRAV1-2 | 28692 | TCRA |
| TRAV12-1 | 28674 | TCRA |
| TRAV12-2 | 28673 | TCRA |
| TRAV12-3 | 28672 | TCRA |
| TRAV13-1 | 28671 | TCRA |
| TRAV13-2 | 28670 | TCRA |
| TRAV14DV4 | 28669 | TCRA |
| TRAV16 | 28667 | TCRA |
| TRAV17 | 28666 | TCRA |
| TRAV18 | 28665 | TCRA |
| TRAV19 | 28664 | TCRA |
| TRAV2 | 28691 | TCRA |
| TRAV20 | 28663 | TCRA |
| TRAV21 | 28662 | TCRA |
| TRAV22 | 28661 | TCRA |
| TRAV23DV6 | 28660 | TCRA |
| TRAV24 | 28659 | TCRA |
| TRAV25 | 28658 | TCRA |
| TRAV26-1 | 28657 | TCRA |
| TRAV26-2 | 28656 | TCRA |
| TRAV27 | 28655 | TCRA |
| TRAV29DV5 | 28653 | TCRA |
| TRAV3 | 28690 | TCRA |
| TRAV30 | 28652 | TCRA |
| TRAV34 | 28648 | TCRA |
| TRAV35 | 28647 | TCRA |
| TRAV36DV7 | 28646 | TCRA |
| TRAV38-1 | 28644 | TCRA |
| TRAV38- | 28643 | TCRA |
| 2DV8 | | |
| TRAV39 | 28642 | TCRA |
| TRAV4 | 28689 | TCRA |
| TRAV40 | 28641 | TCRA |
| TRAV41 | 28640 | TCRA |
| TRAV5 | 28688 | TCRA |
| TRAV7 | 28686 | TCRA |
| TRAV8-1 | 28685 | TCRA |
| TRAV8-2 | 28684 | TCRA |
| TRAV8-3 | 28683 | TCRA |
| TRAV8-4 | 28682 | TCRA |
| TRAV8-6 | 28680 | TCRA |
| TRAV8-7 | 28679 | TCRA |
| TRAV9-1 | 28678 | TCRA |
| TRAV9-2 | 28677 | TCRA |
| TRAJ10 | 28745 | TCRAJ |
| TRAJ11 | 28744 | TCRAJ |
| TRAJ12 | 28743 | TCRAJ |
| TRAJ13 | 28742 | TCRAJ |
| TRAJ14 | 28741 | TCRAJ |
| TRAJ15 | 28740 | TCRAJ |
| TRAJ16 | 28739 | TCRAJ |
| TRAJ17 | 28738 | TCRAJ |
| TRAJ18 | 28737 | TCRAJ |
| TRAJ19 | 28736 | TCRAJ |
| TRAJ20 | 28735 | TCRAJ |
| TRAJ21 | 28734 | TCRAJ |
| TRAJ22 | 28733 | TCRAJ |
| TRAJ23 | 28732 | TCRAJ |
| TRAJ24 | 28731 | TCRAJ |
| TRAJ25 | 28730 | TCRAJ |
| TRAJ26 | 28729 | TCRAJ |
| TRAJ27 | 28728 | TCRAJ |
| TRAJ28 | 28727 | TCRAJ |
| TRAJ29 | 28726 | TCRAJ |
| TRAJ3 | 28752 | TCRAJ |
| TRAJ30 | 28725 | TCRAJ |
| TRAJ31 | 28724 | TCRAJ |
| TRAJ32 | 28723 | TCRAJ |
| TRAJ33 | 28722 | TCRAJ |
| TRAJ34 | 28721 | TCRAJ |
| TRAJ35 | 28720 | TCRAJ |
| TRAJ36 | 28719 | TCRAJ |
| TRAJ37 | 28718 | TCRAJ |
| TRAJ38 | 28717 | TCRAJ |
| TRAJ39 | 28716 | TCRAJ |
| TRAJ4 | 28751 | TCRAJ |
| TRAJ40 | 28715 | TCRAJ |
| TRAJ41 | 28714 | TCRAJ |
| TRAJ42 | 28713 | TCRAJ |
| TRAJ43 | 28712 | TCRAJ |
| TRAJ44 | 28711 | TCRAJ |
| TRAJ45 | 28710 | TCRAJ |
| TRAJ46 | 28709 | TCRAJ |
| TRAJ47 | 28708 | TCRAJ |
| TRAJ48 | 28707 | TCRAJ |
| TRAJ49 | 28706 | TCRAJ |
| TRAJ5 | 28750 | TCRAJ |
| TRAJ50 | 28705 | TCRAJ |
| TRAJ52 | 28703 | TCRAJ |
| TRAJ53 | 28702 | TCRAJ |
| TRAJ54 | 28701 | TCRAJ |
| TRAJ56 | 28699 | TCRAJ |
| TRAJ57 | 28698 | TCRAJ |
| TRAJ58 | 28697 | TCRAJ |
| TRAJ59 | 28696 | TCRAJ |
| TRAJ6 | 28749 | TCRAJ |
| TRAJ61 | 28694 | TCRAJ |
| TRAJ7 | 28748 | TCRAJ |
| TRAJ8 | 28747 | TCRAJ |
| TRAJ9 | 28746 | TCRAJ |
| TRBC2 | 28638 | TCRB |
| TRBJ2-1 | 28629 | TCRB |
| TRBJ2-2 | 28628 | TCRB |
| TRBJ2-2P | 28627 | TCRB |
| TRBJ2-3 | 28626 | TCRB |
| TRBJ2-4 | 28625 | TCRB |
| TRBJ2-5 | 28624 | TCRB |
| TRBJ2-6 | 28623 | TCRB |
| TRBJ2-7 | 28622 | TCRB |
| TRBV1 | 28621 | TCRB |
| TRBV10-1 | 28585 | TCRB |
| TRBV10-2 | 28584 | TCRB |
| TRBV11-1 | 28582 | TCRB |
| TRBV11-2 | 28581 | TCRB |
| TRBV19 | 28568 | TCRB |
| TRBV2 | 28620 | TCRB |
| TRBV20-1 | 28567 | TCRB |
| TRBV21-1 | 28566 | TCRB |
| TRBV23-1 | 28564 | TCRB |
| TRBV24-1 | 28563 | TCRB |
| TRBV25-1 | 28562 | TCRB |
| TRBV27 | 28560 | TCRB |
| TRBV28 | 28559 | TCRB |
| TRBV3-1 | 28619 | TCRB |
| TRBV4-1 | 28617 | TCRB |
| TRBV4-2 | 28616 | TCRB |
| TRBV5-1 | 28614 | TCRB |
| TRBV5-3 | 28612 | TCRB |
| TRBV5-4 | 28611 | TCRB |
| TRBV5-5 | 28610 | TCRB |
| TRBV5-6 | 28609 | TCRB |
| TRBV5-7 | 28608 | TCRB |
| TRBV6-1 | 28606 | TCRB |
| TRBV6-4 | 28603 | TCRB |
| TRBV6-5 | 28602 | TCRB |
| TRBV6-6 | 28601 | TCRB |
| TRBV6-7 | 28600 | TCRB |
| TRBV6-8 | 28599 | TCRB |
| TRBV7-1 | 28597 | TCRB |
| TRBV7-3 | 28595 | TCRB |
| TRBV7-4 | 28594 | TCRB |
| TRBV7-5 | 28593 | TCRB |
| TRBV7-6 | 28592 | TCRB |
| TRBV7-7 | 28591 | TCRB |
| TRBV9 | 28586 | TCRB |
| TRDC | 28526 | TCRD |
| TRDJ1 | 28522 | TCRD |
| TRDJ2 | 28521 | TCRD |
| TRDJ3 | 28520 | TCRD |
| TRDJ4 | 28519 | TCRD |
| TRDV1 | 28518 | TCRD |
| TRDV2 | 28517 | TCRD |
| TRDV3 | 28516 | TCRD |
| TRGC2 | 6967 | TCRG |
| TRGV1 | 6973 | TCRG |
| TRGV10 | 6984 | TCRG |
| TRGV11 | 6985 | TCRG |
| TRGV2 | 6974 | TCRG |
| TRGV3 | 6976 | TCRG |
| TRGV4 | 6977 | TCRG |
| TRGV5 | 6978 | TCRG |
| TRGV7 | 6981 | TCRG |
| TRGV8 | 6982 | TCRG |
| TRGV9 | 6983 | TCRG |
| BRCA1 | 672 | TNF_Waddel Up |
| CASP1 | 834 | TNF_Waddel Up |
| CXCL1 | 2919 | TNF_Waddel Up |
| CXCL2 | 2920 | TNF_Waddel Up |
| GBP1 | 2633 | TNF_Waddel Up |
| GP1BA | 2811 | TNF_Waddel Up |
| IFI44 | 10561 | TNF_Waddel Up |
| IL18 | 3606 | TNF_Waddel Up |
| IL1B | 3553 | TNF_Waddel Up |
| IL1RN | 3557 | TNF_Waddel Up |
| MX1 | 4599 | TNF_Waddel Up |
| NAMPT | 10135 | TNF_Waddel Up |
| NR3C1 | 2908 | TNF_Waddel Up |
| OAS3 | 4940 | TNF_Waddel Up |
| PATJ | 10207 | TNF_Waddel Up |
| SH3BP5 | 9467 | TNF_Waddel Up |
| TAP2 | 6891 | TNF_Waddel Up |
| TNF | 7124 | TNF_Waddel Up |
| TNFAIP3 | 7128 | TNF_Waddel Up |
| TNFRSF11A | 8792 | TNF_Waddel Up |
| WT1 | 7490 | TNF_Waddel Up |
| ACLY | 47 | TNF_Waddel Up |
| ACSL1 | 2180 | TNF_Waddel Up |
| ADGRE2 | 30817 | TNF_Waddel Up |
| AK3 | 50808 | TNF_Waddel Up |
| AKAP10 | 11216 | TNF_Waddel Up |
| AMPD3 | 272 | TNF_Waddel Up |
| APOL3 | 80833 | TNF_Waddel Up |
| ARID3A | 1820 | TNF_Waddel Up |
| ARSE | 415 | TNF_Waddel Up |
| ASAP1 | 50807 | TNF_Waddel Up |
| B4GALT5 | 9334 | TNF_Waddel Up |
| BCL2A1 | 597 | TNF_Waddel Up |
| BHLHE41 | 79365 | TNF_Waddel Up |
| BHMT | 635 | TNF_Waddel Up |
| BIRC3 | 330 | TNF_Waddel Up |
| CALD1 | 800 | TNF_Waddel Up |
| CASP10 | 843 | TNF_Waddel Up |
| CCL15 | 6359 | TNF_Waddel Up |
| CCL20 | 6364 | TNF_Waddel Up |
| CCL23 | 6368 | TNF_Waddel Up |
| CCL3L1 | 6349 | TNF_Waddel Up |
| CD37 | 951 | TNF_Waddel Up |
| CD38 | 952 | TNF_Waddel Up |
| CD83 | 9308 | TNF_Waddel Up |
| CDKN3 | 1033 | TNF_Waddel Up |
| CKB | 1152 | TNF_Waddel Up |
| CR2 | 1380 | TNF_Waddel Up |
| CTNND2 | 1501 | TNF_Waddel Up |
| CXCL3 | 2921 | TNF_Waddel Up |
| CXCL8 | 3576 | TNF_Waddel Up |
| CYP27B1 | 1594 | TNF_Waddel Up |
| DAB2 | 1601 | TNF_Waddel Up |
| EBI3 | 10148 | TNF_Waddel Up |
| EGR1 | 1958 | TNF_Waddel Up |
| EGR2 | 1959 | TNF_Waddel Up |
| EPB41 | 2035 | TNF_Waddel Up |
| EREG | 2069 | TNF_Waddel Up |
| ETAA1 | 54465 | TNF_Waddel Up |
| F3 | 2152 | TNF_Waddel Up |
| FABP1 | 2168 | TNF_Waddel Up |
| FBXL2 | 25827 | TNF_Waddel Up |
| FCER2 | 2208 | TNF_Waddel Up |
| FCGR2A | 2212 | TNF_Waddel Up |
| FLJ11129 | 54674 | TNF_Waddel Up |
| FLNA | 2316 | TNF_Waddel Up |
| G0S2 | 50486 | TNF_Waddel Up |
| GCH1 | 2643 | TNF_Waddel Up |
| GJB2 | 2706 | TNF_Waddel Up |
| GLS | 2744 | TNF_Waddel Up |
| GMIP | 51291 | TNF_Waddel Up |
| GRK3 | 157 | TNF_Waddel Up |
| HCAR3 | 8843 | TNF_Waddel Up |
| HHEX | 3087 | TNF_Waddel Up |
| HOMER2 | 9455 | TNF_Waddel Up |
| HP | 3240 | TNF_Waddel Up |
| ICAM1 | 3383 | TNF_Waddel Up |
| IDO1 | 3620 | TNF_Waddel Up |
| IKBKG | 8517 | TNF_Waddel Up |
| IL16 | 3603 | TNF_Waddel Up |
| IL1A | 3552 | TNF_Waddel Up |
| IL6 | 3569 | TNF_Waddel Up |
| INHBA | 3624 | TNF_Waddel Up |
| INSIG1 | 3638 | TNF_Waddel Up |
| ITGA6 | 3655 | TNF_Waddel Up |
| KITLG | 4254 | TNF_Waddel Up |
| KLF1 | 10661 | TNF_Waddel Up |
| KMO | 8564 | TNF_Waddel Up |
| LGALS3BP | 3959 | TNF_Waddel Up |
| MAP3K4 | 4216 | TNF_Waddel Up |
| MARCKS | 4082 | TNF_Waddel Up |
| MGLL | 11343 | TNF_Waddel Up |
| MMP19 | 4327 | TNF_Waddel Up |
| MN1 | 4330 | TNF_Waddel Up |
| MRPS15 | 64960 | TNF_Waddel Up |
| MSC | 9242 | TNF_Waddel Up |
| MTF1 | 4520 | TNF_Waddel Up |
| NELL2 | 4753 | TNF_Waddel Up |
| NFKB1 | 4790 | TNF_Waddel Up |
| NFKB2 | 4791 | TNF_Waddel Up |
| NFKBIA | 4792 | TNF_Waddel Up |
| NFKBIZ | 64332 | TNF_Waddel Up |
| NKX3-2 | 579 | TNF_Waddel Up |
| PDE4DIP | 9659 | TNF_Waddel Up |
| PDPN | 10630 | TNF_Waddel Up |
| PIAS4 | 51588 | TNF_Waddel Up |
| PLAUR | 5329 | TNF_Waddel Up |
| PTGES | 9536 | TNF_Waddel Up |
| PTGS2 | 5743 | TNF_Waddel Up |
| RELB | 5971 | TNF_Waddel Up |
| RPGR | 6103 | TNF_Waddel Up |
| RPS9 | 6203 | TNF_Waddel Up |
| SDC4 | 6385 | TNF_Waddel Up |
| SERPIND1 | 3053 | TNF_Waddel Up |
| SFRP1 | 6422 | TNF_Waddel Up |
| SLAMF1 | 6504 | TNF_Waddel Up |
| SLC30A4 | 7782 | TNF_Waddel Up |
| SOD2 | 6648 | TNF_Waddel Up |
| SPI1 | 6688 | TNF_Waddel Up |
| SSPN | 8082 | TNF_Waddel Up |
| STAT4 | 6775 | TNF_Waddel Up |
| TAF15 | 8148 | TNF_Waddel Up |
| TBX3 | 6926 | TNF_Waddel Up |
| TFF1 | 7031 | TNF_Waddel Up |
| TNFAIP2 | 7127 | TNF_Waddel Up |
| TRAF1 | 7185 | TNF_Waddel Up |
| TSC22D1 | 8848 | TNF_Waddel Up |
| TYROBP | 7305 | TNF_Waddel Up |
| UBE2C | 11065 | TNF_Waddel Up |
| VEGFA | 7422 | TNF_Waddel Up |
| B4GALT3 | 8703 | Unfolded Protein |
| CALR | 811 | Unfolded Protein |
| CALU | 813 | Unfolded Protein |
| CANX | 821 | Unfolded Protein |
| CDS2 | 8760 | Unfolded Protein |
| CHST12 | 55501 | Unfolded Protein |
| CHST2 | 9435 | Unfolded Protein |
| DERL1 | 79139 | Unfolded Protein |
| DERL2 | 51009 | Unfolded Protein |
| DNAJC3 | 5611 | Unfolded Protein |
| EDEM2 | 55741 | Unfolded Protein |
| EDEM3 | 80267 | Unfolded Protein |
| EMC9 | 51016 | Unfolded Protein |
| ERAP1 | 51752 | Unfolded Protein |
| ERGIC2 | 51290 | Unfolded Protein |
| ERO1L | 30001 | Unfolded Protein |
| EXT1 | 2131 | Unfolded Protein |
| GALNT2 | 2590 | Unfolded Protein |
| GOLT1B | 51026 | Unfolded Protein |
| HERPUD1 | 9709 | Unfolded Protein |
| HYOU1 | 10525 | Unfolded Protein |
| IER3IP1 | 51124 | Unfolded Protein |
| IMPAD1 | 54928 | Unfolded Protein |
| KDELC1 | 79070 | Unfolded Protein |
| KDELR2 | 11014 | Unfolded Protein |
| LMAN2 | 10960 | Unfolded Protein |
| LPGAT1 | 9926 | Unfolded Protein |
| MAN1A1 | 4121 | Unfolded Protein |
| MANEA | 79694 | Unfolded Protein |
| MANF | 7873 | Unfolded Protein |
| NUCB2 | 4925 | Unfolded Protein |
| PDIA4 | 9601 | Unfolded Protein |
| PDIA6 | 10130 | Unfolded Protein |
| PIGK | 10026 | Unfolded Protein |
| PPIB | 5479 | Unfolded Protein |
| SEC24D | 9871 | Unfolded Protein |
| SEC61G | 23480 | Unfolded Protein |
| SPCS3 | 60559 | Unfolded Protein |
| SSR1 | 6745 | Unfolded Protein |
| SSR3 | 6747 | Unfolded Protein |
| TRAM1 | 23471 | Unfolded Protein |
| TRAM2 | 9697 | Unfolded Protein |
| UGGT1 | 56886 | Unfolded Protein |
| XBP1 | 7494 | Unfolded Protein |
| APP | 351 | NLRP3 Inflammasome |
| ATAT1 | 79969 | NLRP3 Inflammasome |
| CARD8 | 22900 | NLRP3 Inflammasome |
| CASP1 | 834 | NLRP3 Inflammasome |
| CASP4 | 837 | NLRP3 Inflammasome |
| CD36 | 948 | NLRP3 Inflammasome |
| CPTP | 80772 | NLRP3 Inflammasome |
| DHX33 | 56919 | NLRP3 Inflammasome |
| EIF2AK2 | 5610 | NLRP3 Inflammasome |
| GBP5 | 115362 | NLRP3 Inflammasome |
| GSDMD | 79792 | NLRP3 Inflammasome |
| HSP90AB1 | 3326 | NLRP3 Inflammasome |
| MEFV | 4210 | NLRP3 Inflammasome |
| NFKB1 | 4790 | NLRP3 Inflammasome |
| NFKB2 | 4791 | NLRP3 Inflammasome |
| NLRC3 | 197358 | NLRP3 Inflammasome |
| NLRP3 | 114548 | NLRP3 Inflammasome |
| P2RX7 | 5027 | NLRP3 Inflammasome |
| PANX1 | 24145 | NLRP3 Inflammasome |
| PSTPIP1 | 9051 | NLRP3 Inflammasome |
| PYCARD | 29108 | NLRP3 Inflammasome |
| PYDC2 | 152138 | NLRP3 Inflammasome |
| RELA | 5970 | NLRP3 Inflammasome |
| SIRT2 | 22933 | NLRP3 Inflammasome |
| SUGT1 | 10910 | NLRP3 Inflammasome |
| TLR4 | 7099 | NLRP3 Inflammasome |
| TLR6 | 10333 | NLRP3 Inflammasome |
| TXN | 7295 | NLRP3 Inflammasome |
| TXNIP | 10628 | NLRP3 Inflammasome |
| USP50 | 373509 | NLRP3 Inflammasome |
| C1QA | 712 | Classical Complement Pathway |
| C1QB | 713 | Classical Complement Pathway |
| C1QC | 714 | Classical Complement Pathway |
| C1R | 715 | Classical Complement Pathway |
| C1S | 716 | Classical Complement Pathway |
| C2 | 717 | Classical Complement Pathway |
| C3 | 718 | Classical Complement Pathway |
| C4A | 720 | Classical Complement Pathway |
| C4B | 721 | Classical Complement Pathway |
| C4B_2 | 100293534 | Classical Complement Pathway |
| C5 | 727 | Classical Complement Pathway |
| C6 | 729 | Classical Complement Pathway |
| C7 | 730 | Classical Complement Pathway |
| C8A | 731 | Classical Complement Pathway |
| C9 | 735 | Classical Complement Pathway |
| C3 | 718 | Alternative Complement Pathway |
| C5 | 727 | Alternative Complement Pathway |
| C6 | 729 | Alternative Complement Pathway |
| C7 | 730 | Alternative Complement Pathway |
| C8A | 731 | Alternative Complement Pathway |
| C9 | 735 | Alternative Complement Pathway |
| CFB | 629 | Alternative Complement Pathway |
| CFD | 1675 | Alternative Complement Pathway |
| CFH | 3075 | Alternative Complement Pathway |
| CFHR5 | 81494 | Alternative Complement Pathway |
| CFP | 5199 | Alternative Complement Pathway |
| CR1 | 1378 | Alternative Complement Pathway |
| GZMM | 3004 | Alternative Complement Pathway |
| MIR520B | 574473 | Alternative Complement Pathway |
| MIR520E | 574461 | Alternative Complement Pathway |
| VSIG4 | 11326 | Alternative Complement Pathway |
| A2M | 2 | Lectin-Induced Complement Pathway |
| C2 | 717 | Lectin-Induced Complement Pathway |
| C3 | 718 | Lectin-Induced Complement Pathway |
| C4A | 720 | Lectin-Induced Complement Pathway |
| C4B | 721 | Lectin-Induced Complement Pathway |
| C4B_2 | 100293534 | Lectin-Induced Complement Pathway |
| C5 | 727 | Lectin-Induced Complement Pathway |
| C6 | 729 | Lectin-Induced Complement Pathway |
| C7 | 730 | Lectin-Induced Complement Pathway |
| C8A | 731 | Lectin-Induced Complement Pathway |
| C9 | 735 | Lectin-Induced Complement Pathway |
| COLEC10 | 10584 | Lectin-Induced Complement Pathway |
| COLEC11 | 78989 | Lectin-Induced Complement Pathway |
| FCN1 | 2219 | Lectin-Induced Complement Pathway |
| FCN2 | 2220 | Lectin-Induced Complement Pathway |
| FCN3 | 8547 | Lectin-Induced Complement Pathway |
| KRT1 | 3848 | Lectin-Induced Complement Pathway |
| MASP1 | 5648 | Lectin-Induced Complement Pathway |
| MASP2 | 10747 | Lectin-Induced Complement Pathway |
| MBL2 | 4153 | Lectin-Induced Complement Pathway |
| SERPING1 | 710 | Lectin-Induced Complement Pathway |

Table of NLRP3 Inflamm and Complement

| geneSymbol | geneEntrezID | GeneSet |
|---|---|---|
| APP | 351 | NLRP3 Inflammasome |
| ATAT1 | 79969 | NLRP3 Inflammasome |
| CARD8 | 22900 | NLRP3 Inflammasome |
| CASP1 | 834 | NLRP3 Inflammasome |
| CASP4 | 837 | NLRP3 Inflammasome |
| CD36 | 948 | NLRP3 Inflammasome |
| CPTP | 80772 | NLRP3 Inflammasome |
| DHX33 | 56919 | NLRP3 Inflammasome |
| EIF2AK2 | 5610 | NLRP3 Inflammasome |
| GBP5 | 115362 | NLRP3 Inflammasome |
| GSDMD | 79792 | NLRP3 Inflammasome |
| HSP90AB1 | 3326 | NLRP3 Inflammasome |
| MEFV | 4210 | NLRP3 Inflammasome |
| NFKB1 | 4790 | NLRP3 Inflammasome |
| NFKB2 | 4791 | NLRP3 Inflammasome |
| NLRC3 | 197358 | NLRP3 Inflammasome |
| NLRP3 | 114548 | NLRP3 Inflammasome |
| P2RX7 | 5027 | NLRP3 Inflammasome |
| PANX1 | 24145 | NLRP3 Inflammasome |
| PSTPIP1 | 9051 | NLRP3 Inflammasome |
| PYCARD | 29108 | NLRP3 Inflammasome |
| PYDC2 | 152138 | NLRP3 Inflammasome |
| RELA | 5970 | NLRP3 Inflammasome |
| SIRT2 | 22933 | NLRP3 Inflammasome |
| SUGT1 | 10910 | NLRP3 Inflammasome |
| TLR4 | 7099 | NLRP3 Inflammasome |
| TLR6 | 10333 | NLRP3 Inflammasome |
| TXN | 7295 | NLRP3 Inflammasome |
| TXNIP | 10628 | NLRP3 Inflammasome |
| USP50 | 373509 | NLRP3 Inflammasome |
| C1QA | 712 | Classical Complement Pathway |
| CIQB | 713 | Classical Complement Pathway |
| C1QC | 714 | Classical Complement Pathway |
| C1R | 715 | Classical Complement Pathway |
| C1S | 716 | Classical Complement Pathway |
| C2 | 717 | Classical Complement Pathway |
| C3 | 718 | Classical Complement Pathway |
| C4A | 720 | Classical Complement Pathway |
| C4B | 721 | Classical Complement Pathway |
| C4B_2 | 100293534 | Classical Complement Pathway |
| C5 | 727 | Classical Complement Pathway |
| C6 | 729 | Classical Complement Pathway |
| C7 | 730 | Classical Complement Pathway |
| C8A | 731 | Classical Complement Pathway |
| C9 | 735 | Classical Complement Pathway |
| C3 | 718 | Alternative Complement Pathway |
| C5 | 727 | Alternative Complement Pathway |
| C6 | 729 | Alternative Complement Pathway |
| C7 | 730 | Alternative Complement Pathway |
| C8A | 731 | Alternative Complement Pathway |
| C9 | 735 | Alternative Complement Pathway |
| CFB | 629 | Alternative Complement Pathway |
| CFD | 1675 | Alternative Complement Pathway |
| CFH | 3075 | Alternative Complement Pathway |
| CFHR5 | 81494 | Alternative Complement Pathway |
| CFP | 5199 | Alternative Complement Pathway |
| CR1 | 1378 | Alternative Complement Pathway |
| GZMM | 3004 | Alternative Complement Pathway |
| MIR520B | 574473 | Alternative Complement Pathway |
| MIR520E | 574461 | Alternative Complement Pathway |
| VSIG4 | 11326 | Alternative Complement Pathway |
| A2M | 2 | Lectin-Induced Complement Pathway |
| C2 | 717 | Lectin-Induced Complement Pathway |
| C3 | 718 | Lectin-Induced Complement Pathway |
| C4A | 720 | Lectin-Induced Complement Pathway |
| C4B | 721 | Lectin-Induced Complement Pathway |
| C4B_2 | 100293534 | Lectin-Induced Complement Pathway |
| C5 | 727 | Lectin-Induced Complement Pathway |
| C6 | 729 | Lectin-Induced Complement Pathway |
| C7 | 730 | Lectin-Induced Complement Pathway |
| C8A | 731 | Lectin-Induced Complement Pathway |
| C9 | 735 | Lectin-Induced Complement Pathway |
| COLEC10 | 10584 | Lectin-Induced Complement Pathway |
| COLEC11 | 78989 | Lectin-Induced Complement Pathway |
| FCN1 | 2219 | Lectin-Induced Complement Pathway |
| FCN2 | 2220 | Lectin-Induced Complement Pathway |
| FCN3 | 8547 | Lectin-Induced Complement Pathway |
| KRT1 | 3848 | Lectin-Induced Complement Pathway |
| MASP1 | 5648 | Lectin-Induced Complement Pathway |
| MASP2 | 10747 | Lectin-Induced Complement Pathway |
| MBL2 | 4153 | Lectin-Induced Complement Pathway |
| SERPING1 | 710 | Lectin-Induced Complement Pathway |

Table of IL6-IL1

| geneSymbol | geneEntrezID | GeneSet |
|---|---|---|
| IL1A | 3552 | Proinflammatory IL-1 Family |
| IL1B | 3553 | Proinflammatory IL-1 Family |
| IL18 | 3606 | Proinflammatory IL-1 Family |
| IL36A | 27179 | Proinflammatory IL-1 Family |
| IL36B | 27177 | Proinflammatory IL-1 Family |
| IL36G | 56300 | Proinflammatory IL-1 Family |
| IL33 | 90865 | Proinflammatory IL-1 Family |
| IL1R1 | 3554 | Proinflammatory IL-1 Family |
| IL1RAP | 3556 | Proinflammatory IL-1 Family |
| IL18R1 | 8809 | Proinflammatory IL-1 Family |
| IL18RAP | 8807 | Proinflammatory IL-1 Family |
| IL6 | 3569 | IL-6R Complex |
| IL6R | 3570 | IL-6R Complex |
| IL6ST | 3572 | IL-6R Complex |

Table of M1 and M2 Macrophages

| geneSymbol | geneEntrezID | GeneSet |
|---|---|---|
| AK3 | 50808 | M1 |
| APOL1 | 8542 | M1 |
| APOL2 | 23780 | M1 |
| APOL3 | 80833 | M1 |
| APOL6 | 80830 | M1 |
| ATF3 | 467 | M1 |
| BCL2A1 | 597 | M1 |
| BIRC3 | 330 | M1 |
| CCL15 | 6359 | M1 |
| CCL19 | 6363 | M1 |
| CCL20 | 6364 | M1 |
| CCL5 | 6352 | M1 |
| CCR7 | 1236 | M1 |
| CHI3L2 | 1117 | M1 |
| CXCL10 | 3627 | M1 |
| CXCL11 | 6373 | M1 |
| CXCL9 | 4283 | M1 |
| EDN1 | 1906 | M1 |
| FAS | 355 | M1 |
| GADD45G | 10912 | M1 |
| HESX1 | 8820 | M1 |
| HSD11B1 | 3290 | M1 |
| IDO1 | 3620 | M1 |
| IGFBP4 | 3487 | M1 |
| IL12B | 3593 | M1 |
| IL15 | 3600 | M1 |
| IL15RA | 3601 | M1 |
| IL2RA | 3559 | M1 |
| IL6 | 3569 | M1 |
| IL7R | 3575 | M1 |
| INHBA | 3624 | M1 |
| IRF1 | 3659 | M1 |
| IRF7 | 3665 | M1 |
| NAMPT | 10135 | M1 |
| OAS2 | 4939 | M1 |
| OASL | 8638 | M1 |
| PDGFA | 5154 | M1 |
| PFKFB3 | 5209 | M1 |
| PFKP | 5214 | M1 |
| PLA1A | 51365 | M1 |
| PSMA2 | 5683 | M1 |
| PSMB9 | 5698 | M1 |
| PSME2 | 5721 | M1 |
| PTX3 | 5806 | M1 |
| SCO2 | 9997 | M1 |
| SLC2A6 | 11182 | M1 |
| SLC31A2 | 1318 | M1 |
| SLC7A5 | 8140 | M1 |
| SLCO5A1 | 81796 | M1 |
| SPHK1 | 8877 | M1 |
| TNF | 7124 | M1 |
| TNFSF10 | 8743 | M1 |
| VCAN | 1462 | M1 |
| XAF1 | 54739 | M1 |
| ADK | 132 | M2 |
| ALOX15 | 246 | M2 |
| CA2 | 760 | M2 |
| CCL13 | 6357 | M2 |
| CCL18 | 6362 | M2 |
| CCL23 | 6368 | M2 |
| CD209 | 30835 | M2 |
| CD36 | 948 | M2 |
| CERK | 64781 | M2 |
| CHN2 | 1124 | M2 |
| CLEC4F | 165530 | M2 |
| CLEC7A | 64581 | M2 |
| CTSC | 1075 | M2 |
| CXCR4 | 7852 | M2 |
| DLC1 | 10395 | M2 |
| EGR2 | 1959 | M2 |
| FGL2 | 10875 | M2 |
| FN1 | 2335 | M2 |
| GAS7 | 8522 | M2 |
| HEXB | 3074 | M2 |
| HNMT | 3176 | M2 |
| HRH1 | 3269 | M2 |
| HS3ST1 | 9957 | M2 |
| HS3ST2 | 9956 | M2 |
| IGF1 | 3479 | M2 |
| LIPA | 3988 | M2 |
| LPAR6 | 10161 | M2 |
| LTA4H | 4048 | M2 |
| MAF | 4094 | M2 |
| MRC1 | 4360 | M2 |
| MS4A4A | 51338 | M2 |
| MS4A6A | 64231 | M2 |
| MSR1 | 4481 | M2 |
| P2RY13 | 53829 | M2 |
| P2RY14 | 9934 | M2 |
| SELENOP | 6414 | M2 |
| SLC38A6 | 145389 | M2 |
| SLC4A7 | 9497 | M2 |
| SLCO2B1 | 11309 | M2 |
| TGFBI | 7045 | M2 |
| TGFBR2 | 7048 | M2 |
| TLR5 | 7100 | M2 |
| TPST2 | 8459 | M2 |

Table of Kotliarov et al. CD 40

| geneSymbol | geneEntrezID | GeneSet |
|---|---|---|
| BUB1B | 701 | CD40act |
| CCL22 | 6367 | CD40act |
| CD58 | 965 | CD40act |
| DBI | 1622 | CD40act |
| DUSP4 | 1846 | CD40act |
| FABP5 | 2171 | CD40act |
| FDPS | 2224 | CD40act |
| FEN1 | 2237 | CD40act |
| GAPDH | 2597 | CD40act |
| GARS | 2617 | CD40act |
| GRHPR | 9380 | CD40act |
| H2AFX | 3014 | CD40act |
| H2AFZ | 3015 | CD40act |
| HMGB2 | 3148 | CD40act |
| HMMR | 3161 | CD40act |
| HPRT1 | 3251 | CD40act |
| IMPDH2 | 3615 | CD40act |
| LDHA | 3939 | CD40act |
| LDHB | 3945 | CD40act |
| LMNB1 | 4001 | CD40act |
| LPXN | 9404 | CD40act |
| MCM2 | 4171 | CD40act |
| MCM3 | 4172 | CD40act |
| MCM6 | 4175 | CD40act |
| MSH6 | 2956 | CD40act |
| MTHFD2 | 10797 | CD40act |
| MYBL2 | 4605 | CD40act |
| NDC80 | 10403 | CD40act |
| NME1 | 4830 | CD40act |
| PAICS | 10606 | CD40act |
| PCNA | 5111 | CD40act |
| PKM | 5315 | CD40act |
| PRDX3 | 10935 | CD40act |
| RFTN1 | 23180 | CD40act |
| RGS10 | 6001 | CD40act |
| SLAMF1 | 6504 | CD40act |
| TK1 | 7083 | CD40act |
| TOP2A | 7153 | CD40act |
| TPX2 | 22974 | CD40act |
| TRAF1 | 7185 | CD40act |
| TUBA1B | 10376 | CD40act |
| TXN | 7295 | CD40act |
| TYMS | 7298 | CD40act |
| UBE2S | 27338 | CD40act |
| VDAC1 | 7416 | CD40act |
| WEE1 | 7465 | CD40act |
| WSB2 | 55884 | CD40act |
| ZWINT | 11130 | CD40act |

Table of Interferons (IFN)

| geneSymbol | geneEntrezID | GeneSet |
|---|---|---|
| ACSL1 | 2180 | IFNA2 Signature |
| ADAR | 103 | IFNA2 Signature |
| AGT | 183 | IFNA2 Signature |
| AIM2 | 9447 | IFNA2 Signature |
| AKAP2 | 11217 | IFNA2 Signature |
| APOBEC3B | 9582 | IFNA2 Signature |
| APOBEC3G | 60489 | IFNA2 Signature |
| APOL3 | 80833 | IFNA2 Signature |
| ATF3 | 467 | IFNA2 Signature |
| ATF5 | 22809 | IFNA2 Signature |
| BAG1 | 573 | IFNA2 Signature |
| BARD1 | 580 | IFNA2 Signature |
| BCL7B | 9275 | IFNA2 Signature |
| BLVRA | 644 | IFNA2 Signature |
| BRCA1 | 672 | IFNA2 Signature |
| BRCA2 | 675 | IFNA2 Signature |
| BST2 | 684 | IFNA2 Signature |
| BUB1 | 699 | IFNA2 Signature |
| C2 | 717 | IFNA2 Signature |
| CACNA1A | 773 | IFNA2 Signature |
| CAD | 790 | IFNA2 Signature |
| CAMK2A | 815 | IFNA2 Signature |
| CASP1 | 834 | IFNA2 Signature |
| CASP10 | 843 | IFNA2 Signature |
| CASP5 | 838 | IFNA2 Signature |
| CBR1 | 873 | IFNA2 Signature |
| CBWD1 | 55871 | IFNA2 Signature |
| CCL13 | 6357 | IFNA2 Signature |
| CCL7 | 6354 | IFNA2 Signature |
| CCL8 | 6355 | IFNA2 Signature |
| CCNA1 | 8900 | IFNA2 Signature |
| CCND2 | 894 | IFNA2 Signature |
| CD2AP | 23607 | IFNA2 Signature |
| CD38 | 952 | IFNA2 Signature |
| CD4 | 920 | IFNA2 Signature |
| CD69 | 969 | IFNA2 Signature |
| CDC42EP1 | 11135 | IFNA2 Signature |
| CDK4 | 1019 | IFNA2 Signature |
| CDKN1A | 1026 | IFNA2 Signature |
| CFB | 629 | IFNA2 Signature |
| CH25H | 9023 | IFNA2 Signature |
| CHKA | 1119 | IFNA2 Signature |
| CNTN6 | 27255 | IFNA2 Signature |
| COL3A1 | 1281 | IFNA2 Signature |
| CTSL | 1514 | IFNA2 Signature |
| CXCL10 | 3627 | IFNA2 Signature |
| CXCL11 | 6373 | IFNA2 Signature |
| CXCL9 | 4283 | IFNA2 Signature |
| CXCR2 | 3579 | IFNA2 Signature |
| CYP2J2 | 1573 | IFNA2 Signature |
| DAB2 | 1601 | IFNA2 Signature |
| DEFB1 | 1672 | IFNA2 Signature |
| DLL1 | 28514 | IFNA2 Signature |
| DSC2 | 1824 | IFNA2 Signature |
| DUSP5 | 1847 | IFNA2 Signature |
| DUSP7 | 1849 | IFNA2 Signature |
| DYNLT1 | 6993 | IFNA2 Signature |
| DYSF | 8291 | IFNA2 Signature |
| ECE1 | 1889 | IFNA2 Signature |
| EDN1 | 1906 | IFNA2 Signature |
| EIF2AK2 | 5610 | IFNA2 Signature |
| EIF2B1 | 1967 | IFNA2 Signature |
| EIF4ENIF1 | 56478 | IFNA2 Signature |
| ENPP2 | 5168 | IFNA2 Signature |
| EPB41 | 2035 | IFNA2 Signature |
| ETV4 | 2118 | IFNA2 Signature |
| F8 | 2157 | IFNA2 Signature |
| FAF1 | 11124 | IFNA2 Signature |
| FAS | 355 | IFNA2 Signature |
| FGF1 | 2246 | IFNA2 Signature |
| FLNA | 2316 | IFNA2 Signature |
| FOXO1 | 2308 | IFNA2 Signature |
| FTL | 2512 | IFNA2 Signature |
| FUT4 | 2526 | IFNA2 Signature |
| GADD45B | 4616 | IFNA2 Signature |
| GBAP1 | 2630 | IFNA2 Signature |
| GBP1 | 2633 | IFNA2 Signature |
| GBP2 | 2634 | IFNA2 Signature |
| GCH1 | 2643 | IFNA2 Signature |
| GCNT1 | 2650 | IFNA2 Signature |
| GLB1 | 2720 | IFNA2 Signature |
| GLS | 2744 | IFNA2 Signature |
| GMPR | 2766 | IFNA2 Signature |
| GPR161 | 23432 | IFNA2 Signature |
| GUK1 | 2987 | IFNA2 Signature |
| HBG2 | 3048 | IFNA2 Signature |
| HCAR3 | 8843 | IFNA2 Signature |
| HIST2H2AA3 | 8337 | IFNA2 Signature |
| HLA-DOA | 3111 | IFNA2 Signature |
| HLA-DRB5 | 3127 | IFNA2 Signature |
| HS6ST1 | 9394 | IFNA2 Signature |
| HSP90AA1 | 3320 | IFNA2 Signature |
| IDO1 | 3620 | IFNA2 Signature |
| IFI16 | 3428 | IFNA2 Signature |
| IFI27 | 3429 | IFNA2 Signature |
| IFI35 | 3430 | IFNA2 Signature |
| IFI44 | 10561 | IFNA2 Signature |
| IFI44L | 10964 | IFNA2 Signature |
| IFI6 | 2537 | IFNA2 Signature |
| IFIT1 | 3434 | IFNA2 Signature |
| IFIT5 | 24138 | IFNA2 Signature |
| IFITM1 | 8519 | IFNA2 Signature |
| IFITM2 | 10581 | IFNA2 Signature |
| IFITM3 | 10410 | IFNA2 Signature |
| IFNG | 3458 | IFNA2 Signature |
| IFRD1 | 3475 | IFNA2 Signature |
| IGL | 3535 | IFNA2 Signature |
| IKBKG | 8517 | IFNA2 Signature |
| IL15 | 3600 | IFNA2 Signature |
| IL15RA | 3601 | IFNA2 Signature |
| IL1RN | 3557 | IFNA2 Signature |
| IL6 | 3569 | IFNA2 Signature |
| INPPL1 | 3636 | IFNA2 Signature |
| IRF2 | 3660 | IFNA2 Signature |
| IRF7 | 3665 | IFNA2 Signature |
| ISG15 | 9636 | IFNA2 Signature |
| ISG20 | 3669 | IFNA2 Signature |
| ITIH2 | 3698 | IFNA2 Signature |
| JAK2 | 3717 | IFNA2 Signature |
| JUP | 3728 | IFNA2 Signature |
| KCNA3 | 3738 | IFNA2 Signature |
| KDELR2 | 11014 | IFNA2 Signature |
| KIF20B | 9585 | IFNA2 Signature |
| KLF6 | 1316 | IFNA2 Signature |
| KPNB1 | 3837 | IFNA2 Signature |
| KRT8 | 3856 | IFNA2 Signature |
| LAG3 | 3902 | IFNA2 Signature |
| LAMP3 | 27074 | IFNA2 Signature |
| LAP3 | 51056 | IFNA2 Signature |
| LEPR | 3953 | IFNA2 Signature |
| LGALS2 | 3957 | IFNA2 Signature |
| LGALS3BP | 3959 | IFNA2 Signature |
| LGALS9 | 3965 | IFNA2 Signature |
| LGMN | 5641 | IFNA2 Signature |
| LMNB1 | 4001 | IFNA2 Signature |
| LMO2 | 4005 | IFNA2 Signature |
| LY6E | 4061 | IFNA2 Signature |
| MAP2K5 | 5607 | IFNA2 Signature |
| MCL1 | 4170 | IFNA2 Signature |
| MED1 | 5469 | IFNA2 Signature |
| MGLL | 11343 | IFNA2 Signature |
| MMP16 | 4325 | IFNA2 Signature |
| MNDA | 4332 | IFNA2 Signature |
| MRPS15 | 64960 | IFNA2 Signature |
| MSR1 | 4481 | IFNA2 Signature |
| MX1 | 4599 | IFNA2 Signature |
| MX2 | 4600 | IFNA2 Signature |
| MYD88 | 4615 | IFNA2 Signature |
| NAMPT | 10135 | IFNA2 Signature |
| NFE2L3 | 9603 | IFNA2 Signature |
| NKTR | 4820 | IFNA2 Signature |
| NMI | 9111 | IFNA2 Signature |
| NR3C1 | 2908 | IFNA2 Signature |
| NUB1 | 51667 | IFNA2 Signature |
| NUPR1 | 26471 | IFNA2 Signature |
| OAS1 | 4938 | IFNA2 Signature |
| OAS2 | 4939 | IFNA2 Signature |
| OAS3 | 4940 | IFNA2 Signature |
| OSBPL1A | 114876 | IFNA2 Signature |
| PATJ | 10207 | IFNA2 Signature |
| PDGFB | 5155 | IFNA2 Signature |
| PDGFRL | 5157 | IFNA2 Signature |
| PGGT1B | 5229 | IFNA2 Signature |
| PKD2 | 5311 | IFNA2 Signature |
| PLSCR1 | 5359 | IFNA2 Signature |
| PMAIP1 | 5366 | IFNA2 Signature |
| PML | 5371 | IFNA2 Signature |
| PRKRA | 8575 | IFNA2 Signature |
| PSMB9 | 5698 | IFNA2 Signature |
| PTCH1 | 5727 | IFNA2 Signature |
| RBCK1 | 10616 | IFNA2 Signature |
| RET | 5979 | IFNA2 Signature |
| RGS1 | 5996 | IFNA2 Signature |
| RGS6 | 9628 | IFNA2 Signature |
| RPS9 | 6203 | IFNA2 Signature |
| RTP4 | 64108 | IFNA2 Signature |
| SAT1 | 6303 | IFNA2 Signature |
| SCARB2 | 950 | IFNA2 Signature |
| SERPING1 | 710 | IFNA2 Signature |
| SIT1 | 27240 | IFNA2 Signature |
| SLAMF1 | 6504 | IFNA2 Signature |
| SOCS1 | 8651 | IFNA2 Signature |
| SP100 | 6672 | IFNA2 Signature |
| SP110 | 3431 | IFNA2 Signature |
| SP140 | 11262 | IFNA2 Signature |
| SPIB | 6689 | IFNA2 Signature |
| ST3GAL5 | 8869 | IFNA2 Signature |
| STAP1 | 26228 | IFNA2 Signature |
| STAT1 | 6772 | IFNA2 Signature |
| STAT2 | 6773 | IFNA2 Signature |
| STX11 | 8676 | IFNA2 Signature |
| SUPT3H | 8464 | IFNA2 Signature |
| SYN2 | 6854 | IFNA2 Signature |
| TAF5L | 27097 | IFNA2 Signature |
| TAP1 | 6890 | IFNA2 Signature |
| TAP2 | 6891 | IFNA2 Signature |
| TARBP1 | 6894 | IFNA2 Signature |
| TCN2 | 6948 | IFNA2 Signature |
| TFDP2 | 7029 | IFNA2 Signature |
| TGM1 | 7051 | IFNA2 Signature |
| TLR3 | 7098 | IFNA2 Signature |
| TLR7 | 51284 | IFNA2 Signature |
| TNFRSF11A | 8792 | IFNA2 Signature |
| TNFSF10 | 8743 | IFNA2 Signature |
| TNFSF6 | 356 | IFNA2 Signature |
| TNK2 | 10188 | IFNA2 Signature |
| TOR1B | 27348 | IFNA2 Signature |
| TRA2B | 6434 | IFNA2 Signature |
| TRD | 6964 | IFNA2 Signature |
| TRIM21 | 6737 | IFNA2 Signature |
| TRIM22 | 10346 | IFNA2 Signature |
| TRIM26 | 7726 | IFNA2 Signature |
| TRIM34 | 53840 | IFNA2 Signature |
| TRIM38 | 10475 | IFNA2 Signature |
| UBA7 | 7318 | IFNA2 Signature |
| UBE2L6 | 9246 | IFNA2 Signature |
| UBE2S | 27338 | IFNA2 Signature |
| UBE3A | 7337 | IFNA2 Signature |
| UNC93B1 | 81622 | IFNA2 Signature |
| USP18 | 11274 | IFNA2 Signature |
| VAMP5 | 10791 | IFNA2 Signature |
| WARS | 7453 | IFNA2 Signature |
| WT1 | 7490 | IFNA2 Signature |
| XAF1 | 54739 | IFNA2 Signature |
| ACKR3 | 57007 | IFNB1 ALTERNATIVE |
| | | PW Increased transcripts |
| ACOD1 | 730249 | IFNB1 ALTERNATIVE |
| | | PW Increased transcripts |
| ADORA2B | 136 | IFNB1 ALTERNATIVE |
| | | PW Increased transcripts |
| AEN | 64782 | IFNB1 ALTERNATIVE |
| | | PW Increased transcripts |
| AMBP | 259 | IFNB1 ALTERNATIVE |
| | | PW Increased transcripts |
| ARG1 | 383 | IFNB1 ALTERNATIVE |
| | | PW Increased transcripts |
| ARG2 | 384 | IFNB1 ALTERNATIVE |
| | | PW Increased transcripts |
| ARHGAP31 | 57514 | IFNB1 ALTERNATIVE |
| | | PW Increased transcripts |
| ASPA | 443 | IFNB1 ALTERNATIVE |
| | | PW Increased transcripts |
| B4GALT5 | 9334 | IFNB1 ALTERNATIVE |
| | | PW Increased transcripts |
| BCL3 | 602 | IFNB1 ALTERNATIVE |
| | | PW Increased transcripts |
| BYSL | 705 | IFNB1 ALTERNATIVE |
| | | PW Increased transcripts |
| CA13 | 377677 | IFNB1 ALTERNATIVE |
| | | PW Increased transcripts |
| CAMKK2 | 10645 | IFNB1 ALTERNATIVE |
| | | PW Increased transcripts |
| CCL2 | 6347 | IFNB1 ALTERNATIVE |
| | | PW Increased transcripts |
| CCL3L3 | 6349 | IFNB1 ALTERNATIVE |
| | | PW Increased transcripts |
| CCL4 | 6351 | IFNB1 ALTERNATIVE |
| | | PW Increased transcripts |
| CCL7 | 6354 | IFNB1 ALTERNATIVE |
| | | PW Increased transcripts |
| CCRL2 | 9034 | IFNB1 ALTERNATIVE |
| | | PW Increased transcripts |
| CD14 | 929 | IFNB1 ALTERNATIVE |
| | | PW Increased transcripts |
| CD207 | 50489 | IFNB1 ALTERNATIVE |
| | | PW Increased transcripts |
| CD86 | 942 | IFNB1 ALTERNATIVE |
| | | PW Increased transcripts |
| CDC42EP2 | 10435 | IFNB1 ALTERNATIVE |
| | | PW Increased transcripts |
| CDKN1A | 1026 | IFNB1 ALTERNATIVE |
| | | PW Increased transcripts |
| CDR2 | 1039 | IFNB1 ALTERNATIVE |
| | | PW Increased transcripts |
| CLEC4E | 26253 | IFNB1 ALTERNATIVE |
| | | PW Increased transcripts |
| CLEC6A | 93978 | IFNB1 ALTERNATIVE |
| | | PW Increased transcripts |
| COQ10B | 80219 | IFNB1 ALTERNATIVE |
| | | PW Increased transcripts |
| CRYAA | 1409 | IFNB1 ALTERNATIVE |
| | | PW Increased transcripts |
| CSTA | 1475 | IFNB1 ALTERNATIVE |
| | | PW Increased transcripts |
| CTPS1 | 1503 | IFNB1 ALTERNATIVE |
| | | PW Increased transcripts |
| CXCL2 | 2920 | IFNB1 ALTERNATIVE |
| | | PW Increased transcripts |
| CXCL3 | 2921 | IFNB1 ALTERNATIVE |
| | | PW Increased transcripts |
| DNMT3L | 29947 | IFNB1 ALTERNATIVE |
| | | PW Increased transcripts |
| DOT1L | 84444 | IFNB1 ALTERNATIVE |
| | | PW Increased transcripts |
| DRAM1 | 55332 | IFNB1 ALTERNATIVE |
| | | PW Increased transcripts |
| DUSP16 | 80824 | IFNB1 ALTERNATIVE |
| | | PW Increased transcripts |
| EEF1E1 | 9521 | IFNB1 ALTERNATIVE |
| | | PW Increased transcripts |
| EPHA4 | 2043 | IFNB1 ALTERNATIVE |
| | | PW Increased transcripts |
| ETS2 | 2114 | IFNB1 ALTERNATIVE |
| | | PW Increased transcripts |
| EXOC3L4 | 91828 | IFNB1 ALTERNATIVE |
| | | PW Increased transcripts |
| F3 | 2152 | IFNB1 ALTERNATIVE |
| | | PW Increased transcripts |
| FAM20C | 56975 | IFNB1 ALTERNATIVE |
| | | PW Increased transcripts |
| FCRL5 | 83416 | IFNB1 ALTERNATIVE |
| | | PW Increased transcripts |
| FFAR2 | 2867 | IFNB1 ALTERNATIVE |
| | | PW Increased transcripts |
| FMNL2 | 114793 | IFNB1 ALTERNATIVE |
| | | PW Increased transcripts |
| FPR2 | 2358 | IFNB1 ALTERNATIVE |
| | | PW Increased transcripts |
| GFPT1 | 2673 | IFNB1 ALTERNATIVE |
| | | PW Increased transcripts |
| Gk | 2710 | IFNB1 ALTERNATIVE |
| | | PW Increased transcripts |
| GPR84 | 53831 | IFNB1 ALTERNATIVE |
| | | PW Increased transcripts |
| GRWD1 | 83743 | IFNB1 ALTERNATIVE |
| | | PW Increased transcripts |
| HBEGF | 1839 | IFNB1 ALTERNATIVE |
| | | PW Increased transcripts |
| HCAR2 | 338442 | IFNB1 ALTERNATIVE |
| | | PW Increased transcripts |
| HEATR1 | 55127 | IFNB1 ALTERNATIVE |
| | | PW Increased transcripts |
| HIVEP3 | 59269 | IFNB1 ALTERNATIVE |
| | | PW Increased transcripts |
| HSPA1A | 3303 | IFNB1 ALTERNATIVE |
| | | PW Increased transcripts |
| HSPA1B | 3304 | IFNB1 ALTERNATIVE |
| | | PW Increased transcripts |
| ICAM1 | 3383 | IFNB1 ALTERNATIVE |
| | | PW Increased transcripts |
| ID1 | 3397 | IFNB1 ALTERNATIVE |
| | | PW Increased transcripts |
| IER3 | 8870 | IFNB1 ALTERNATIVE |
| | | PW Increased transcripts |
| IFI16 | 3428 | IFNB1 ALTERNATIVE |
| | | PW Increased transcripts |
| IKBKE | 9641 | IFNB1 ALTERNATIVE |
| | | PW Increased transcripts |
| IL1A | 3552 | IFNB1 ALTERNATIVE |
| | | PW Increased transcripts |
| IL1B | 3553 | IFNB1 ALTERNATIVE |
| | | PW Increased transcripts |
| IL1RN | 3557 | IFNB1 ALTERNATIVE |
| | | PW Increased transcripts |
| IL6 | 3569 | IFNB1 ALTERNATIVE |
| | | PW Increased transcripts |
| IRAK3 | 11213 | IFNB1 ALTERNATIVE |
| | | PW Increased transcripts |
| ITGA5 | 3678 | IFNB1 ALTERNATIVE |
| | | PW Increased transcripts |
| ITGAX | 3687 | IFNB1 ALTERNATIVE |
| | | PW Increased transcripts |
| KPNA2 | 3838 | IFNB1 ALTERNATIVE |
| | | PW Increased transcripts |
| MAFF | 23764 | IFNB1 ALTERNATIVE |
| | | PW Increased transcripts |
| MARCKSL1 | 65108 | IFNB1 ALTERNATIVE |
| | | PW Increased transcripts |
| MARCO | 8685 | IFNB1 ALTERNATIVE |
| | | PW Increased transcripts |
| MEFV | 4210 | IFNB1 ALTERNATIVE |
| | | PW Increased transcripts |
| MMP14 | 4323 | IFNB1 ALTERNATIVE |
| | | PW Increased transcripts |
| MT1A | 4489 | IFNB1 ALTERNATIVE |
| | | PW Increased transcripts |
| MT2A | 4502 | IFNB1 ALTERNATIVE |
| | | PW Increased transcripts |
| MYBPC2 | 4606 | IFNB1 ALTERNATIVE |
| | | PW Increased transcripts |
| NAB2 | 4665 | IFNB1 ALTERNATIVE |
| | | PW Increased transcripts |
| NOCT | 25819 | IFNB1 ALTERNATIVE |
| | | PW Increased transcripts |
| NOP16 | 51491 | IFNB1 ALTERNATIVE |
| | | PW Increased transcripts |
| NOP2 | 4839 | IFNB1 ALTERNATIVE |
| | | PW Increased transcripts |
| NR1H3 | 10062 | IFNB1 ALTERNATIVE |
| | | PW Increased transcripts |
| NR4A1 | 3164 | IFNB1 ALTERNATIVE |
| | | PW Increased transcripts |
| OLR1 | 4973 | IFNB1 ALTERNATIVE |
| | | PW Increased transcripts |
| OSM | 5008 | IFNB1 ALTERNATIVE |
| | | PW Increased transcripts |
| PDIA6 | 10130 | IFNB1 ALTERNATIVE |
| | | PW Increased transcripts |
| PHLDA1 | 22822 | IFNB1 ALTERNATIVE |
| | | PW Increased transcripts |
| PHLDB1 | 23187 | IFNB1 ALTERNATIVE |
| | | PW Increased transcripts |
| PIM1 | 5292 | IFNB1 ALTERNATIVE |
| | | PW Increased transcripts |
| POGK | 57645 | IFNB1 ALTERNATIVE |
| | | PW Increased transcripts |
| PPRC1 | 23082 | IFNB1 ALTERNATIVE |
| | | PW Increased transcripts |
| PROK2 | 60675 | IFNB1 ALTERNATIVE |
| | | PW Increased transcripts |
| PTGES | 9536 | IFNB1 ALTERNATIVE |
| | | PW Increased transcripts |
| PTGIR | 5739 | IFNB1 ALTERNATIVE |
| | | PW Increased transcripts |
| PVR | 5817 | IFNB1 ALTERNATIVE |
| | | PW Increased transcripts |
| RAB20 | 55647 | IFNB1 ALTERNATIVE |
| | | PW Increased transcripts |
| RAI14 | 26064 | IFNB1 ALTERNATIVE |
| | | PW Increased transcripts |
| RELB | 5971 | IFNB1 ALTERNATIVE |
| | | PW Increased transcripts |
| RND1 | 27289 | IFNB1 ALTERNATIVE |
| | | PW Increased transcripts |
| RRP12 | 23223 | IFNB1 ALTERNATIVE |
| | | PW Increased transcripts |
| RRS1 | 23212 | IFNB1 ALTERNATIVE |
| | | PW Increased transcripts |
| SAA1 | 6288 | IFNB1 ALTERNATIVE |
| | | PW Increased transcripts |
| SCAMP1 | 9522 | IFNB1 ALTERNATIVE |
| | | PW Increased transcripts |
| SDC4 | 6385 | IFNB1 ALTERNATIVE |
| | | PW Increased transcripts |
| SERPINB2 | 5055 | IFNB1 ALTERNATIVE |
| | | PW Increased transcripts |
| SERPINE1 | 5054 | IFNB1 ALTERNATIVE |
| | | PW Increased transcripts |
| SHMT1 | 6470 | IFNB1 ALTERNATIVE |
| | | PW Increased transcripts |
| SLAMF8 | 56833 | IFNB1 ALTERNATIVE |
| | | PW Increased transcripts |
| SLC12A4 | 6560 | IFNB1 ALTERNATIVE |
| | | PW Increased transcripts |
| SLC15A3 | 51296 | IFNB1 ALTERNATIVE |
| | | PW Increased transcripts |
| SLC16A10 | 117247 | IFNB1 ALTERNATIVE |
| | | PW Increased transcripts |
| SLC20A1 | 6574 | IFNB1 ALTERNATIVE |
| | | PW Increased transcripts |
| SLC25A25 | 114789 | IFNB1 ALTERNATIVE |
| | | PW Increased transcripts |
| SLC25A33 | 84275 | IFNB1 ALTERNATIVE |
| | | PW Increased transcripts |
| SLC2A6 | 11182 | IFNB1 ALTERNATIVE |
| | | PW Increased transcripts |
| SLC39A14 | 23516 | IFNB1 ALTERNATIVE |
| | | PW Increased transcripts |
| SLC7A2 | 6542 | IFNB1 ALTERNATIVE |
| | | PW Increased transcripts |
| SLC7A5 | 8140 | IFNB1 ALTERNATIVE |
| | | PW Increased transcripts |
| SNX18 | 112574 | IFNB1 ALTERNATIVE |
| | | PW Increased transcripts |
| SOCS3 | 9021 | IFNB1 ALTERNATIVE |
| | | PW Increased transcripts |
| SOD2 | 6648 | IFNB1 ALTERNATIVE |
| | | PW Increased transcripts |
| SUSD6 | 9766 | IFNB1 ALTERNATIVE |
| | | PW Increased transcripts |
| TFEC | 22797 | IFNB1 ALTERNATIVE |
| | | PW Increased transcripts |
| TFRC | 7037 | IFNB1 ALTERNATIVE |
| | | PW Increased transcripts |
| TGM2 | 7052 | IFNB1 ALTERNATIVE |
| | | PW Increased transcripts |
| TIMM8A | 1678 | IFNB1 ALTERNATIVE |
| | | PW Increased transcripts |
| TLNRD1 | 59274 | IFNB1 ALTERNATIVE |
| | | PW Increased transcripts |
| TLR2 | 7097 | IFNB1 ALTERNATIVE |
| | | PW Increased transcripts |
| TMA16 | 55319 | IFNB1 ALTERNATIVE |
| | | PW Increased transcripts |
| TNF | 7124 | IFNB1 ALTERNATIVE |
| | | PW Increased transcripts |
| TNFAIP2 | 7127 | IFNB1 ALTERNATIVE |
| | | PW Increased transcripts |
| TNFAIP3 | 7128 | IFNB1 ALTERNATIVE |
| | | PW Increased transcripts |
| TNFSF14 | 8740 | IFNB1 ALTERNATIVE |
| | | PW Increased transcripts |
| TREM1 | 54210 | IFNB1 ALTERNATIVE |
| | | PW Increased transcripts |
| TREML4 | 285852 | IFNB1 ALTERNATIVE |
| | | PW Increased transcripts |
| TRIM13 | 10206 | IFNB1 ALTERNATIVE |
| | | PW Increased transcripts |
| TRMT61A | 115708 | IFNB1 ALTERNATIVE |
| | | PW Increased transcripts |
| TXNRD1 | 7296 | IFNB1 ALTERNATIVE |
| | | PW Increased transcripts |
| URB2 | 9816 | IFNB1 ALTERNATIVE |
| | | PW Increased transcripts |
| ZNF503 | 84858 | IFNB1 ALTERNATIVE |
| | | PW Increased transcripts |
| ACLY | 47 | IFNB1 Signature |
| ACSL1 | 2180 | IFNB1 Signature |
| ADAM19 | 8728 | IFNB1 Signature |
| ADAP2 | 55803 | IFNB1 Signature |
| ADAR | 103 | IFNB1 Signature |
| ADGRE2 | 30817 | IFNB1 Signature |
| ADM | 133 | IFNB1 Signature |
| AFF3 | 3899 | IFNB1 Signature |
| AGT | 183 | IFNB1 Signature |
| AIM2 | 9447 | IFNB1 Signature |
| AKAP10 | 11216 | IFNB1 Signature |
| AKAP2 | 11217 | IFNB1 Signature |
| ALOX12 | 239 | IFNB1 Signature |
| ALOX5 | 240 | IFNB1 Signature |
| ANXA4 | 307 | IFNB1 Signature |
| APOBEC3B | 9582 | IFNB1 Signature |
| APOBEC3G | 60489 | IFNB1 Signature |
| APOL3 | 80833 | IFNB1 Signature |
| ATF3 | 467 | IFNB1 Signature |
| ATF5 | 22809 | IFNB1 Signature |
| ATM | 472 | IFNB1 Signature |
| ATP13A1 | 57130 | IFNB1 Signature |
| B4GAT1 | 11041 | IFNB1 Signature |
| BAG1 | 573 | IFNB1 Signature |
| BAK1 | 578 | IFNB1 Signature |
| BARD1 | 580 | IFNB1 Signature |
| BCL11A | 53335 | IFNB1 Signature |
| BCL7B | 9275 | IFNB1 Signature |
| BGN | 633 | IFNB1 Signature |
| BLNK | 29760 | IFNB1 Signature |
| BLVRA | 644 | IFNB1 Signature |
| BLZF1 | 8548 | IFNB1 Signature |
| BRCA1 | 672 | IFNB1 Signature |
| BRCA2 | 675 | IFNB1 Signature |
| BST2 | 684 | IFNB1 Signature |
| BUB1 | 699 | IFNB1 Signature |
| C3AR1 | 719 | IFNB1 Signature |
| CACNA1A | 773 | IFNB1 Signature |
| CAD | 790 | IFNB1 Signature |
| CALD1 | 800 | IFNB1 Signature |
| CAMK2A | 815 | IFNB1 Signature |
| CAPN2 | 824 | IFNB1 Signature |
| CASP1 | 834 | IFNB1 Signature |
| CASP10 | 843 | IFNB1 Signature |
| CASP5 | 838 | IFNB1 Signature |
| CBR1 | 873 | IFNB1 Signature |
| CBWD1 | 55871 | IFNB1 Signature |
| CCL13 | 6357 | IFNB1 Signature |
| CCL3L1 | 6349 | IFNB1 Signature |
| CCL4 | 6351 | IFNB1 Signature |
| CCL7 | 6354 | IFNB1 Signature |
| CCL8 | 6355 | IFNB1 Signature |
| CCNA1 | 8900 | IFNB1 Signature |
| CCND2 | 894 | IFNB1 Signature |
| CCR1 | 1230 | IFNB1 Signature |
| CCR5 | 1234 | IFNB1 Signature |
| CCRL2 | 9034 | IFNB1 Signature |
| CD163 | 9332 | IFNB1 Signature |
| CD164 | 8763 | IFNB1 Signature |
| CD2AP | 23607 | IFNB1 Signature |
| CD38 | 952 | IFNB1 Signature |
| CD4 | 920 | IFNB1 Signature |
| CD59 | 966 | IFNB1 Signature |
| CD69 | 969 | IFNB1 Signature |
| CD72 | 971 | IFNB1 Signature |
| CD86 | 942 | IFNB1 Signature |
| CDK17 | 5128 | IFNB1 Signature |
| CDKN1A | 1026 | IFNB1 Signature |
| CENPA | 1058 | IFNB1 Signature |
| CENPE | 1062 | IFNB1 Signature |
| CFB | 629 | IFNB1 Signature |
| CFLAR | 8837 | IFNB1 Signature |
| CH25H | 9023 | IFNB1 Signature |
| CHI3L2 | 1117 | IFNB1 Signature |
| CHKA | 1119 | IFNB1 Signature |
| CISH | 1154 | IFNB1 Signature |
| CKB | 1152 | IFNB1 Signature |
| CMAHP | 8418 | IFNB1 Signature |
| CNTN6 | 27255 | IFNB1 Signature |
| CNTRL | 11064 | IFNB1 Signature |
| COL3A1 | 1281 | IFNB1 Signature |
| COX17 | 10063 | IFNB1 Signature |
| CSF2RB | 1439 | IFNB1 Signature |
| CTSL | 1514 | IFNB1 Signature |
| CXCL10 | 3627 | IFNB1 Signature |
| CXCL11 | 6373 | IFNB1 Signature |
| CXCL2 | 2920 | IFNB1 Signature |
| CXCL9 | 4283 | IFNB1 Signature |
| CXCR2 | 3579 | IFNB1 Signature |
| CYBB | 1536 | IFNB1 Signature |
| CYP19A1 | 1588 | IFNB1 Signature |
| CYP2J2 | 1573 | IFNB1 Signature |
| DAB2 | 1601 | IFNB1 Signature |
| DEFA1 | 1667 | IFNB1 Signature |
| DEFB1 | 1672 | IFNB1 Signature |
| DHFR | 1719 | IFNB1 Signature |
| DLL1 | 28514 | IFNB1 Signature |
| DMXL1 | 1657 | IFNB1 Signature |
| DNMT1 | 1786 | IFNB1 Signature |
| DRAP1 | 10589 | IFNB1 Signature |
| DSC2 | 1824 | IFNB1 Signature |
| DUSP5 | 1847 | IFNB1 Signature |
| DUSP7 | 1849 | IFNB1 Signature |
| DYNLT1 | 6993 | IFNB1 Signature |
| DYSF | 8291 | IFNB1 Signature |
| E2F1 | 1869 | IFNB1 Signature |
| ECE1 | 1889 | IFNB1 Signature |
| EDN1 | 1906 | IFNB1 Signature |
| EGR1 | 1958 | IFNB1 Signature |
| EIF2AK2 | 5610 | IFNB1 Signature |
| EIF2B1 | 1967 | IFNB1 Signature |
| EIF4ENIF1 | 56478 | IFNB1 Signature |
| ELF1 | 1997 | IFNB1 Signature |
| ELF4 | 2000 | IFNB1 Signature |
| ENPP2 | 5168 | IFNB1 Signature |
| EPB41 | 2035 | IFNB1 Signature |
| ETV4 | 2118 | IFNB1 Signature |
| ETV6 | 2120 | IFNB1 Signature |
| F8 | 2157 | IFNB1 Signature |
| FAF1 | 11124 | IFNB1 Signature |
| FAS | 355 | IFNB1 Signature |
| FBXW2 | 26190 | IFNB1 Signature |
| FCGR1A | 2209 | IFNB1 Signature |
| FCMR | 9214 | IFNB1 Signature |
| FGF1 | 2246 | IFNB1 Signature |
| FLNA | 2316 | IFNB1 Signature |
| FMR1 | 2332 | IFNB1 Signature |
| FOXO1 | 2308 | IFNB1 Signature |
| FPR2 | 2358 | IFNB1 Signature |
| FTL | 2512 | IFNB1 Signature |
| FUT4 | 2526 | IFNB1 Signature |
| GADD45B | 4616 | IFNB1 Signature |
| GBAP1 | 2630 | IFNB1 Signature |
| GBP1 | 2633 | IFNB1 Signature |
| GBP2 | 2634 | IFNB1 Signature |
| GCH1 | 2643 | IFNB1 Signature |
| GCNT1 | 2650 | IFNB1 Signature |
| GLS | 2744 | IFNB1 Signature |
| GMPR | 2766 | IFNB1 Signature |
| GPI | 2821 | IFNB1 Signature |
| GPR161 | 23432 | IFNB1 Signature |
| GUK1 | 2987 | IFNB1 Signature |
| HBG2 | 3048 | IFNB1 Signature |
| HCAR3 | 8843 | IFNB1 Signature |
| HHEX | 3087 | IFNB1 Signature |
| HIST2H2AA3 | 8337 | IFNB1 Signature |
| HK2 | 3099 | IFNB1 Signature |
| HLA-DOA | 3111 | IFNB1 Signature |
| HS6ST1 | 9394 | IFNB1 Signature |
| HSP90AA1 | 3320 | IFNB1 Signature |
| HSPA1A | 3303 | IFNB1 Signature |
| HSPA1L | 3305 | IFNB1 Signature |
| IDO1 | 3620 | IFNB1 Signature |
| IFI16 | 3428 | IFNB1 Signature |
| IFI27 | 3429 | IFNB1 Signature |
| IFI35 | 3430 | IFNB1 Signature |
| IFI44 | 10561 | IFNB1 Signature |
| IFI6 | 2537 | IFNB1 Signature |
| IFIT1 | 3434 | IFNB1 Signature |
| IFIT5 | 24138 | IFNB1 Signature |
| IFITM1 | 8519 | IFNB1 Signature |
| IFITM2 | 10581 | IFNB1 Signature |
| IFITM3 | 10410 | IFNB1 Signature |
| IFNG | 3458 | IFNB1 Signature |
| IFRD1 | 3475 | IFNB1 Signature |
| IGL | 3535 | IFNB1 Signature |
| IKBKE | 9641 | IFNB1 Signature |
| IKBKG | 8517 | IFNB1 Signature |
| IL15 | 3600 | IFNB1 Signature |
| IL15RA | 3601 | IFNB1 Signature |
| IL18BP | 10068 | IFNB1 Signature |
| IL18R1 | 8809 | IFNB1 Signature |
| IL1RN | 3557 | IFNB1 Signature |
| IL6 | 3569 | IFNB1 Signature |
| IL7 | 3574 | IFNB1 Signature |
| INPP5D | 3635 | IFNB1 Signature |
| INPPL1 | 3636 | IFNB1 Signature |
| IRF1 | 3659 | IFNB1 Signature |
| IRF2 | 3660 | IFNB1 Signature |
| IRF4 | 3662 | IFNB1 Signature |
| IRF7 | 3665 | IFNB1 Signature |
| IRF9 | 10379 | IFNB1 Signature |
| ISG15 | 9636 | IFNB1 Signature |
| ISG20 | 3669 | IFNB1 Signature |
| ITGAL | 3683 | IFNB1 Signature |
| ITGAX | 3687 | IFNB1 Signature |
| JAK2 | 3717 | IFNB1 Signature |
| JCHAIN | 3512 | IFNB1 Signature |
| JUP | 3728 | IFNB1 Signature |
| KCNA3 | 3738 | IFNB1 Signature |
| KCNMB1 | 3779 | IFNB1 Signature |
| KDELR2 | 11014 | IFNB1 Signature |
| KIF20B | 9585 | IFNB1 Signature |
| KLF2 | 10365 | IFNB1 Signature |
| KLF6 | 1316 | IFNB1 Signature |
| KLRB1 | 3820 | IFNB1 Signature |
| KPNB1 | 3837 | IFNB1 Signature |
| KRT8 | 3856 | IFNB1 Signature |
| LAG3 | 3902 | IFNB1 Signature |
| LAMP3 | 27074 | IFNB1 Signature |
| LANCL1 | 10314 | IFNB1 Signature |
| LAP3 | 51056 | IFNB1 Signature |
| LBR | 3930 | IFNB1 Signature |
| LEPR | 3953 | IFNB1 Signature |
| LGALS2 | 3957 | IFNB1 Signature |
| LGALS3BP | 3959 | IFNB1 Signature |
| LGALS9 | 3965 | IFNB1 Signature |
| LGMN | 5641 | IFNB1 Signature |
| LILRA1 | 11024 | IFNB1 Signature |
| LINC00597 | 81698 | IFNB1 Signature |
| LMNB1 | 4001 | IFNB1 Signature |
| LMO2 | 4005 | IFNB1 Signature |
| LTA | 4049 | IFNB1 Signature |
| LTB4R | 1241 | IFNB1 Signature |
| LY6E | 4061 | IFNB1 Signature |
| LYN | 4067 | IFNB1 Signature |
| MAP2K5 | 5607 | IFNB1 Signature |
| MAP3K8 | 1326 | IFNB1 Signature |
| MARCKS | 4082 | IFNB1 Signature |
| MBNL | 4154 | IFNB1 Signature |
| MCL1 | 4170 | IFNB1 Signature |
| MED1 | 5469 | IFNB1 Signature |
| MEF2A | 4205 | IFNB1 Signature |
| MFHAS1 | 9258 | IFNB1 Signature |
| MGLL | 11343 | IFNB1 Signature |
| MNDA | 4332 | IFNB1 Signature |
| MRPS15 | 64960 | IFNB1 Signature |
| MS4A7 | 58475 | IFNB1 Signature |
| MSR1 | 4481 | IFNB1 Signature |
| MX1 | 4599 | IFNB1 Signature |
| MX2 | 4600 | IFNB1 Signature |
| MYD88 | 4615 | IFNB1 Signature |
| NAMPT | 10135 | IFNB1 Signature |
| NAPSA | 9476 | IFNB1 Signature |
| NBN | 4683 | IFNB1 Signature |
| NCF1 | 653361 | IFNB1 Signature |
| NCOA2 | 10499 | IFNB1 Signature |
| NEBL | 10529 | IFNB1 Signature |
| NEK4 | 6787 | IFNB1 Signature |
| NFE2L3 | 9603 | IFNB1 Signature |
| NKTR | 4820 | IFNB1 Signature |
| NMI | 9111 | IFNB1 Signature |
| NOTCH1 | 4851 | IFNB1 Signature |
| NR3C1 | 2908 | IFNB1 Signature |
| NR4A3 | 8013 | IFNB1 Signature |
| NUB1 | 51667 | IFNB1 Signature |
| NUPR1 | 26471 | IFNB1 Signature |
| OAS1 | 4938 | IFNB1 Signature |
| OAS2 | 4939 | IFNB1 Signature |
| OAS3 | 4940 | IFNB1 Signature |
| PATJ | 10207 | IFNB1 Signature |
| PAX5 | 5079 | IFNB1 Signature |
| PAX8 | 7849 | IFNB1 Signature |
| PDE4B | 5142 | IFNB1 Signature |
| PDGFB | 5155 | IFNB1 Signature |
| PDGFRL | 5157 | IFNB1 Signature |
| PFKFB3 | 5209 | IFNB1 Signature |
| PFKP | 5214 | IFNB1 Signature |
| PIM2 | 11040 | IFNB1 Signature |
| PKD2 | 5311 | IFNB1 Signature |
| PLEK | 5341 | IFNB1 Signature |
| PLSCR1 | 5359 | IFNB1 Signature |
| PMAIP1 | 5366 | IFNB1 Signature |
| PML | 5371 | IFNB1 Signature |
| PMS2 | 5395 | IFNB1 Signature |
| PPP2R2A | 5520 | IFNB1 Signature |
| PRKAG1 | 5571 | IFNB1 Signature |
| PRKRA | 8575 | IFNB1 Signature |
| PRKX | 5613 | IFNB1 Signature |
| PSMB8 | 5696 | IFNB1 Signature |
| PSMB9 | 5698 | IFNB1 Signature |
| PTCH1 | 5727 | IFNB1 Signature |
| PTGER2 | 5732 | IFNB1 Signature |
| RALB | 5899 | IFNB1 Signature |
| RASGRP1 | 10125 | IFNB1 Signature |
| RBBP6 | 5930 | IFNB1 Signature |
| RBCK1 | 10616 | IFNB1 Signature |
| RERE | 473 | IFNB1 Signature |
| RGS1 | 5996 | IFNB1 Signature |
| RGS6 | 9628 | IFNB1 Signature |
| RIN1 | 9610 | IFNB1 Signature |
| RIPK1 | 8737 | IFNB1 Signature |
| RIPK3 | 11035 | IFNB1 Signature |
| RIPOR2 | 9750 | IFNB1 Signature |
| RNF114 | 55905 | IFNB1 Signature |
| RPS6KA5 | 9252 | IFNB1 Signature |
| RPS9 | 6203 | IFNB1 Signature |
| RRBP1 | 6238 | IFNB1 Signature |
| RTP4 | 64108 | IFNB1 Signature |
| SAT1 | 6303 | IFNB1 Signature |
| SCARB2 | 950 | IFNB1 Signature |
| SDS | 10993 | IFNB1 Signature |
| SELL | 6402 | IFNB1 Signature |
| SERPIND1 | 3053 | IFNB1 Signature |
| SERPING1 | 710 | IFNB1 Signature |
| SFTPB | 6439 | IFNB1 Signature |
| SIDT2 | 51092 | IFNB1 Signature |
| SIT1 | 27240 | IFNB1 Signature |
| SLAMF1 | 6504 | IFNB1 Signature |
| SMO | 6608 | IFNB1 Signature |
| SNX2 | 6643 | IFNB1 Signature |
| SOCS1 | 8651 | IFNB1 Signature |
| SOS1 | 6654 | IFNB1 Signature |
| SP100 | 6672 | IFNB1 Signature |
| SP110 | 3431 | IFNB1 Signature |
| SP140 | 11262 | IFNB1 Signature |
| SPIB | 6689 | IFNB1 Signature |
| SPTA1 | 6708 | IFNB1 Signature |
| SPTLC2 | 9517 | IFNB1 Signature |
| SRRM2 | 23524 | IFNB1 Signature |
| SSB | 6741 | IFNB1 Signature |
| ST3GAL5 | 8869 | IFNB1 Signature |
| STAP1 | 26228 | IFNB1 Signature |
| STAT1 | 6772 | IFNB1 Signature |
| STAT2 | 6773 | IFNB1 Signature |
| STOML2 | 30968 | IFNB1 Signature |
| STX11 | 8676 | IFNB1 Signature |
| SUPT3H | 8464 | IFNB1 Signature |
| TANK | 10010 | IFNB1 Signature |
| TAP1 | 6890 | IFNB1 Signature |
| TAP2 | 6891 | IFNB1 Signature |
| TAPBP | 6892 | IFNB1 Signature |
| TARBP1 | 6894 | IFNB1 Signature |
| TBX21 | 30009 | IFNB1 Signature |
| TCN2 | 6948 | IFNB1 Signature |
| TFDP2 | 7029 | IFNB1 Signature |
| TFF1 | 7031 | IFNB1 Signature |
| TGM1 | 7051 | IFNB1 Signature |
| THY1 | 7070 | IFNB1 Signature |
| TLR1 | 7096 | IFNB1 Signature |
| TLR3 | 7098 | IFNB1 Signature |
| TLR7 | 51284 | IFNB1 Signature |
| TNFAIP2 | 7127 | IFNB1 Signature |
| TNFRSF11A | 8792 | IFNB1 Signature |
| TNFSF10 | 8743 | IFNB1 Signature |
| TNFSF6 | 356 | IFNB1 Signature |
| TNK2 | 10188 | IFNB1 Signature |
| TOR1B | 27348 | IFNB1 Signature |
| TRA2B | 6434 | IFNB1 Signature |
| TRD | 6964 | IFNB1 Signature |
| TRG | 6965 | IFNB1 Signature |
| TRIM21 | 6737 | IFNB1 Signature |
| TRIM22 | 10346 | IFNB1 Signature |
| TRIM26 | 7726 | IFNB1 Signature |
| TRIM34 | 53840 | IFNB1 Signature |
| TRIM38 | 10475 | IFNB1 Signature |
| TSPAN15 | 23555 | IFNB1 Signature |
| TXK | 7294 | IFNB1 Signature |
| UBA7 | 7318 | IFNB1 Signature |
| UBE2L6 | 9246 | IFNB1 Signature |
| UBE2S | 27338 | IFNB1 Signature |
| UBE3A | 7337 | IFNB1 Signature |
| UBQLN2 | 29978 | IFNB1 Signature |
| UNC93B1 | 81622 | IFNB1 Signature |
| USP15 | 9958 | IFNB1 Signature |
| USP18 | 11274 | IFNB1 Signature |
| USP25 | 29761 | IFNB1 Signature |
| USPL1 | 10208 | IFNB1 Signature |
| UVRAG | 7405 | IFNB1 Signature |
| VAMP5 | 10791 | IFNB1 Signature |
| WARS | 7453 | IFNB1 Signature |
| WIPF1 | 7456 | IFNB1 Signature |
| WT1 | 7490 | IFNB1 Signature |
| XAF1 | 54739 | IFNB1 Signature |
| ZNF107 | 51427 | IFNB1 Signature |
| ACLY | 47 | IFNG Signature |
| ACSL1 | 2180 | IFNG Signature |
| AFF2 | 2334 | IFNG Signature |
| AIM2 | 9447 | IFNG Signature |
| AKAP10 | 11216 | IFNG Signature |
| APOL3 | 80833 | IFNG Signature |
| ATF3 | 467 | IFNG Signature |
| ATM | 472 | IFNG Signature |
| C1QB | 713 | IFNG Signature |
| C4A | 720 | IFNG Signature |
| CALD1 | 800 | IFNG Signature |
| CASP1 | 834 | IFNG Signature |
| CASP10 | 843 | IFNG Signature |
| CCL8 | 6355 | IFNG Signature |
| CCND2 | 894 | IFNG Signature |
| CCR5 | 1234 | IFNG Signature |
| CD38 | 952 | IFNG Signature |
| CDKN1A | 1026 | IFNG Signature |
| CFB | 629 | IFNG Signature |
| CKB | 1152 | IFNG Signature |
| CLEC10A | 10462 | IFNG Signature |
| CPT1B | 1375 | IFNG Signature |
| CSF2RB | 1439 | IFNG Signature |
| CTNND2 | 1501 | IFNG Signature |
| CXCL10 | 3627 | IFNG Signature |
| CXCL11 | 6373 | IFNG Signature |
| CXCL9 | 4283 | IFNG Signature |
| CYBB | 1536 | IFNG Signature |
| EDN1 | 1906 | IFNG Signature |
| EPB41 | 2035 | IFNG Signature |
| ETAA1 | 54465 | IFNG Signature |
| ETV4 | 2118 | IFNG Signature |
| F8 | 2157 | IFNG Signature |
| FAS | 355 | IFNG Signature |
| FBLN1 | 2192 | IFNG Signature |
| FBXL2 | 25827 | IFNG Signature |
| FCGR1A | 2209 | IFNG Signature |
| FLII | 2314 | IFNG Signature |
| GADD45B | 4616 | IFNG Signature |
| GBP1 | 2633 | IFNG Signature |
| GBP2 | 2634 | IFNG Signature |
| GCH1 | 2643 | IFNG Signature |
| GCNT1 | 2650 | IFNG Signature |
| GLS | 2744 | IFNG Signature |
| GSTM5 | 2949 | IFNG Signature |
| HBG2 | 3048 | IFNG Signature |
| HHEX | 3087 | IFNG Signature |
| HP | 3240 | IFNG Signature |
| ICAM1 | 3383 | IFNG Signature |
| IDO1 | 3620 | IFNG Signature |
| IFI27 | 3429 | IFNG Signature |
| IFI44 | 10561 | IFNG Signature |
| IL15 | 3600 | IFNG Signature |
| IL15RA | 3601 | IFNG Signature |
| IL18BP | 10068 | IFNG Signature |
| IL1A | 3552 | IFNG Signature |
| IL7 | 3574 | IFNG Signature |
| IRF1 | 3659 | IFNG Signature |
| IRF8 | 3394 | IFNG Signature |
| JAK2 | 3717 | IFNG Signature |
| JCHAIN | 3512 | IFNG Signature |
| KLF2 | 10365 | IFNG Signature |
| LAP3 | 51056 | IFNG Signature |
| LIMK2 | 3985 | IFNG Signature |
| LMNB1 | 4001 | IFNG Signature |
| MMP25 | 64386 | IFNG Signature |
| MRPS15 | 64960 | IFNG Signature |
| MSR1 | 4481 | IFNG Signature |
| NET1 | 10276 | IFNG Signature |
| NIN | 51199 | IFNG Signature |
| NKTR | 4820 | IFNG Signature |
| NLRP1 | 22861 | IFNG Signature |
| NR3C1 | 2908 | IFNG Signature |
| OAS1 | 4938 | IFNG Signature |
| OAS3 | 4940 | IFNG Signature |
| P2RY13 | 53829 | IFNG Signature |
| PCDH9 | 5101 | IFNG Signature |
| PLA2G4C | 8605 | IFNG Signature |
| PLEK | 5341 | IFNG Signature |
| POLR2B | 5431 | IFNG Signature |
| PSMB9 | 5698 | IFNG Signature |
| PTCH1 | 5727 | IFNG Signature |
| RALB | 5899 | IFNG Signature |
| RGS1 | 5996 | IFNG Signature |
| SERPIND1 | 3053 | IFNG Signature |
| SERPING1 | 710 | IFNG Signature |
| SFTPB | 6439 | IFNG Signature |
| SLAMF1 | 6504 | IFNG Signature |
| SLC1A5 | 6510 | IFNG Signature |
| SOCS1 | 8651 | IFNG Signature |
| SP100 | 6672 | IFNG Signature |
| SPRY4 | 81848 | IFNG Signature |
| SRRM2 | 23524 | IFNG Signature |
| STAT1 | 6772 | IFNG Signature |
| STAT2 | 6773 | IFNG Signature |
| STX11 | 8676 | IFNG Signature |
| TAP1 | 6890 | IFNG Signature |
| TAP2 | 6891 | IFNG Signature |
| TBX21 | 30009 | IFNG Signature |
| TENM1 | 10178 | IFNG Signature |
| TFF1 | 7031 | IFNG Signature |
| TNFAIP2 | 7127 | IFNG Signature |
| TNFSF10 | 8743 | IFNG Signature |
| UBD | 10537 | IFNG Signature |
| UBE2C | 11065 | IFNG Signature |
| UBE2L6 | 9246 | IFNG Signature |
| UBE3A | 7337 | IFNG Signature |
| VAMP5 | 10791 | IFNG Signature |
| VSNL1 | 7447 | IFNG Signature |
| WARS | 7453 | IFNG Signature |
| XRN1 | 54464 | IFNG Signature |
| ABCB10 | 23456 | IFNW1 Signature |
| ACLY | 47 | IFNW1 Signature |
| ACSL1 | 2180 | IFNW1 Signature |
| ADAR | 103 | IFNW1 Signature |
| ADM | 133 | IFNW1 Signature |
| AGT | 183 | IFNW1 Signature |
| AIM2 | 9447 | IFNW1 Signature |
| AKAP10 | 11216 | IFNW1 Signature |
| AKAP2 | 11217 | IFNW1 Signature |
| ALOX12 | 239 | IFNW1 Signature |
| ANXA4 | 307 | IFNW1 Signature |
| APOBEC3B | 9582 | IFNW1 Signature |
| APOBEC3G | 60489 | IFNW1 Signature |
| APOL3 | 80833 | IFNW1 Signature |
| ATF3 | 467 | IFNW1 Signature |
| ATF5 | 22809 | IFNW1 Signature |
| ATM | 472 | IFNW1 Signature |
| B4GAT1 | 11041 | IFNW1 Signature |
| BAG1 | 573 | IFNW1 Signature |
| BARD1 | 580 | IFNW1 Signature |
| BCL11A | 53335 | IFNW1 Signature |
| BCL7B | 9275 | IFNW1 Signature |
| BLVRA | 644 | IFNW1 Signature |
| BLZF1 | 8548 | IFNW1 Signature |
| BRCA1 | 672 | IFNW1 Signature |
| BRCA2 | 675 | IFNW1 Signature |
| BRD4 | 23476 | IFNW1 Signature |
| BST2 | 684 | IFNW1 Signature |
| C3AR1 | 719 | IFNW1 Signature |
| CAD | 790 | IFNW1 Signature |
| CALD1 | 800 | IFNW1 Signature |
| CAMK2A | 815 | IFNW1 Signature |
| CAPN2 | 824 | IFNW1 Signature |
| CASK | 8573 | IFNW1 Signature |
| CASP1 | 834 | IFNW1 Signature |
| CASP10 | 843 | IFNW1 Signature |
| CASP5 | 838 | IFNW1 Signature |
| CBR1 | 873 | IFNW1 Signature |
| CBWD1 | 55871 | IFNW1 Signature |
| CCL13 | 6357 | IFNW1 Signature |
| CCL3L1 | 6349 | IFNW1 Signature |
| CCL7 | 6354 | IFNW1 Signature |
| CCL8 | 6355 | IFNW1 Signature |
| CCNA1 | 8900 | IFNW1 Signature |
| CCND2 | 894 | IFNW1 Signature |
| CCR1 | 1230 | IFNW1 Signature |
| CCR5 | 1234 | IFNW1 Signature |
| CCR7 | 1236 | IFNW1 Signature |
| CCRL2 | 9034 | IFNW1 Signature |
| CD164 | 8763 | IFNW1 Signature |
| CD2AP | 23607 | IFNW1 Signature |
| CD38 | 952 | IFNW1 Signature |
| CD4 | 920 | IFNW1 Signature |
| CD47 | 961 | IFNW1 Signature |
| CD59 | 966 | IFNW1 Signature |
| CD69 | 969 | IFNW1 Signature |
| CDKN1A | 1026 | IFNW1 Signature |
| CENPE | 1062 | IFNW1 Signature |
| CFB | 629 | IFNW1 Signature |
| CFLAR | 8837 | IFNW1 Signature |
| CHKA | 1119 | IFNW1 Signature |
| CKB | 1152 | IFNW1 Signature |
| CMAHP | 8418 | IFNW1 Signature |
| CNTN6 | 27255 | IFNW1 Signature |
| CNTRL | 11064 | IFNW1 Signature |
| COL3A1 | 1281 | IFNW1 Signature |
| CSF2RB | 1439 | IFNW1 Signature |
| CTSL | 1514 | IFNW1 Signature |
| CXCL10 | 3627 | IFNW1 Signature |
| CXCL11 | 6373 | IFNW1 Signature |
| CXCL9 | 4283 | IFNW1 Signature |
| CXCR2 | 3579 | IFNW1 Signature |
| CYBB | 1536 | IFNW1 Signature |
| CYP19A1 | 1588 | IFNW1 Signature |
| CYP2J2 | 1573 | IFNW1 Signature |
| DEFB1 | 1672 | IFNW1 Signature |
| DLL1 | 28514 | IFNW1 Signature |
| DSC2 | 1824 | IFNW1 Signature |
| DUSP5 | 1847 | IFNW1 Signature |
| DUSP7 | 1849 | IFNW1 Signature |
| DYNLT1 | 6993 | IFNW1 Signature |
| DYSF | 8291 | IFNW1 Signature |
| E2F1 | 1869 | IFNW1 Signature |
| ECE1 | 1889 | IFNW1 Signature |
| EDN1 | 1906 | IFNW1 Signature |
| EGR1 | 1958 | IFNW1 Signature |
| EIF2AK2 | 5610 | IFNW1 Signature |
| EIF2B1 | 1967 | IFNW1 Signature |
| EIF4ENIF1 | 56478 | IFNW1 Signature |
| ENPP2 | 5168 | IFNW1 Signature |
| EPB41 | 2035 | IFNW1 Signature |
| ERCC4 | 2072 | IFNW1 Signature |
| ETV4 | 2118 | IFNW1 Signature |
| ETV6 | 2120 | IFNW1 Signature |
| F8 | 2157 | IFNW1 Signature |
| FAF1 | 11124 | IFNW1 Signature |
| FAS | 355 | IFNW1 Signature |
| FCER1G | 2207 | IFNW1 Signature |
| FGF1 | 2246 | IFNW1 Signature |
| FGF13 | 2258 | IFNW1 Signature |
| FGL2 | 10875 | IFNW1 Signature |
| FLNA | 2316 | IFNW1 Signature |
| FMR1 | 2332 | IFNW1 Signature |
| FOXO1 | 2308 | IFNW1 Signature |
| FTL | 2512 | IFNW1 Signature |
| FUT4 | 2526 | IFNW1 Signature |
| GADD45B | 4616 | IFNW1 Signature |
| GBAP1 | 2630 | IFNW1 Signature |
| GBP1 | 2633 | IFNW1 Signature |
| GBP2 | 2634 | IFNW1 Signature |
| GCH1 | 2643 | IFNW1 Signature |
| GCNT1 | 2650 | IFNW1 Signature |
| GLB1 | 2720 | IFNW1 Signature |
| GLS | 2744 | IFNW1 Signature |
| GMPR | 2766 | IFNW1 Signature |
| GPR161 | 23432 | IFNW1 Signature |
| GSTM5 | 2949 | IFNW1 Signature |
| GUK1 | 2987 | IFNW1 Signature |
| HBG2 | 3048 | IFNW1 Signature |
| HHEX | 3087 | IFNW1 Signature |
| HIST2H2AA3 | 8337 | IFNW1 Signature |
| HLA-DOA | 3111 | IFNW1 Signature |
| HS6ST1 | 9394 | IFNW1 Signature |
| HSP90AA1 | 3320 | IFNW1 Signature |
| HSPA1A | 3303 | IFNW1 Signature |
| IDO1 | 3620 | IFNW1 Signature |
| IFI16 | 3428 | IFNW1 Signature |
| IFI27 | 3429 | IFNW1 Signature |
| IFI35 | 3430 | IFNW1 Signature |
| IFI44 | 10561 | IFNW1 Signature |
| IFI6 | 2537 | IFNW1 Signature |
| IFIT1 | 3434 | IFNW1 Signature |
| IFIT5 | 24138 | IFNW1 Signature |
| IFITM1 | 8519 | IFNW1 Signature |
| IFITM2 | 10581 | IFNW1 Signature |
| IFITM3 | 10410 | IFNW1 Signature |
| IFRD1 | 3475 | IFNW1 Signature |
| IGL | 3535 | IFNW1 Signature |
| IKBKG | 8517 | IFNW1 Signature |
| IL15 | 3600 | IFNW1 Signature |
| IL15RA | 3601 | IFNW1 Signature |
| IL18R1 | 8809 | IFNW1 Signature |
| IL1RN | 3557 | IFNW1 Signature |
| IL6 | 3569 | IFNW1 Signature |
| IL7 | 3574 | IFNW1 Signature |
| INPPL1 | 3636 | IFNW1 Signature |
| IRF1 | 3659 | IFNW1 Signature |
| IRF2 | 3660 | IFNW1 Signature |
| IRF7 | 3665 | IFNW1 Signature |
| IRF8 | 3394 | IFNW1 Signature |
| ISG15 | 9636 | IFNW1 Signature |
| ISG20 | 3669 | IFNW1 Signature |
| ITIH2 | 3698 | IFNW1 Signature |
| JAK2 | 3717 | IFNW1 Signature |
| JCHAIN | 3512 | IFNW1 Signature |
| JUP | 3728 | IFNW1 Signature |
| KCNA3 | 3738 | IFNW1 Signature |
| KDELR2 | 11014 | IFNW1 Signature |
| KIF20B | 9585 | IFNW1 Signature |
| KLF6 | 1316 | IFNW1 Signature |
| KPNB1 | 3837 | IFNW1 Signature |
| KRT8 | 3856 | IFNW1 Signature |
| LAG3 | 3902 | IFNW1 Signature |
| LAMP3 | 27074 | IFNW1 Signature |
| LAP3 | 51056 | IFNW1 Signature |
| LEPR | 3953 | IFNW1 Signature |
| LGALS2 | 3957 | IFNW1 Signature |
| LGALS3BP | 3959 | IFNW1 Signature |
| LGALS9 | 3965 | IFNW1 Signature |
| LGMN | 5641 | IFNW1 Signature |
| LINC00597 | 81698 | IFNW1 Signature |
| LMNB1 | 4001 | IFNW1 Signature |
| LMO2 | 4005 | IFNW1 Signature |
| LY6E | 4061 | IFNW1 Signature |
| LYN | 4067 | IFNW1 Signature |
| MAP2K5 | 5607 | IFNW1 Signature |
| MARCKS | 4082 | IFNW1 Signature |
| MBNL1 | 4154 | IFNW1 Signature |
| MCL1 | 4170 | IFNW1 Signature |
| MED1 | 5469 | IFNW1 Signature |
| MEF2A | 4205 | IFNW1 Signature |
| MGLL | 11343 | IFNW1 Signature |
| MLF1 | 4291 | IFNW1 Signature |
| MMP16 | 4325 | IFNW1 Signature |
| MNDA | 4332 | IFNW1 Signature |
| MRPS15 | 64960 | IFNW1 Signature |
| MS4A7 | 58475 | IFNW1 Signature |
| MSR1 | 4481 | IFNW1 Signature |
| MX1 | 4599 | IFNW1 Signature |
| MX2 | 4600 | IFNW1 Signature |
| MYD88 | 4615 | IFNW1 Signature |
| NAMPT | 10135 | IFNW1 Signature |
| NCF1 | 653361 | IFNW1 Signature |
| NFE2L3 | 9603 | IFNW1 Signature |
| NKTR | 4820 | IFNW1 Signature |
| NMI | 9111 | IFNW1 Signature |
| NPTX1 | 4884 | IFNW1 Signature |
| NR3C1 | 2908 | IFNW1 Signature |
| NUB1 | 51667 | IFNW1 Signature |
| NUPR1 | 26471 | IFNW1 Signature |
| OAS1 | 4938 | IFNW1 Signature |
| OAS2 | 4939 | IFNW1 Signature |
| OAS3 | 4940 | IFNW1 Signature |
| OSBPL1A | 114876 | IFNW1 Signature |
| PATJ | 10207 | IFNW1 Signature |
| PAX8 | 7849 | IFNW1 Signature |
| PDGFB | 5155 | IFNW1 Signature |
| PDGFRL | 5157 | IFNW1 Signature |
| PKD2 | 5311 | IFNW1 Signature |
| PLEK | 5341 | IFNW1 Signature |
| PLSCR1 | 5359 | IFNW1 Signature |
| PMAIP1 | 5366 | IFNW1 Signature |
| PML | 5371 | IFNW1 Signature |
| PPP2R2A | 5520 | IFNW1 Signature |
| PRKAG1 | 5571 | IFNW1 Signature |
| PRKRA | 8575 | IFNW1 Signature |
| PSMB9 | 5698 | IFNW1 Signature |
| PTCH1 | 5727 | IFNW1 Signature |
| PTGER2 | 5732 | IFNW1 Signature |
| RALB | 5899 | IFNW1 Signature |
| RBBP6 | 5930 | IFNW1 Signature |
| RBCK1 | 10616 | IFNW1 Signature |
| RERE | 473 | IFNW1 Signature |
| RGS1 | 5996 | IFNW1 Signature |
| RGS6 | 9628 | IFNW1 Signature |
| RPS6KA5 | 9252 | IFNW1 Signature |
| RTP4 | 64108 | IFNW1 Signature |
| SAT1 | 6303 | IFNW1 Signature |
| SCARB2 | 950 | IFNW1 Signature |
| SDS | 10993 | IFNW1 Signature |
| SELL | 6402 | IFNW1 Signature |
| SERPIND1 | 3053 | IFNW1 Signature |
| SERPING1 | 710 | IFNW1 Signature |
| SFT2D2 | 375035 | IFNW1 Signature |
| SIT1 | 27240 | IFNW1 Signature |
| SLC30A4 | 7782 | IFNW1 Signature |
| SOCS1 | 8651 | IFNW1 Signature |
| SOS1 | 6654 | IFNW1 Signature |
| SP100 | 6672 | IFNW1 Signature |
| SP110 | 3431 | IFNW1 Signature |
| SP140 | 11262 | IFNW1 Signature |
| SPIB | 6689 | IFNW1 Signature |
| SRRM2 | 23524 | IFNW1 Signature |
| ST3GAL5 | 8869 | IFNW1 Signature |
| STAP1 | 26228 | IFNW1 Signature |
| STAT1 | 6772 | IFNW1 Signature |
| STAT2 | 6773 | IFNW1 Signature |
| STX11 | 8676 | IFNW1 Signature |
| SUPT3H | 8464 | IFNW1 Signature |
| TAP1 | 6890 | IFNW1 Signature |
| TAP2 | 6891 | IFNW1 Signature |
| TARBP1 | 6894 | IFNW1 Signature |
| TBX21 | 30009 | IFNW1 Signature |
| TCN2 | 6948 | IFNW1 Signature |
| TFDP2 | 7029 | IFNW1 Signature |
| TFF1 | 7031 | IFNW1 Signature |
| TGM1 | 7051 | IFNW1 Signature |
| THY1 | 7070 | IFNW1 Signature |
| TLR3 | 7098 | IFNW1 Signature |
| TLR7 | 51284 | IFNW1 Signature |
| TNFAIP3 | 7128 | IFNW1 Signature |
| TNFRSF11A | 8792 | IFNW1 Signature |
| TNFSF10 | 8743 | IFNW1 Signature |
| TNFSF6 | 356 | IFNW1 Signature |
| TNK2 | 10188 | IFNW1 Signature |
| TOR1B | 27348 | IFNW1 Signature |
| TRA2B | 6434 | IFNW1 Signature |
| TRD | 6964 | IFNW1 Signature |
| TRIM21 | 6737 | IFNW1 Signature |
| TRIM22 | 10346 | IFNW1 Signature |
| TRIM34 | 53840 | IFNW1 Signature |
| TRIM38 | 10475 | IFNW1 Signature |
| UBA7 | 7318 | IFNW1 Signature |
| UBE2C | 11065 | IFNW1 Signature |
| UBE2L6 | 9246 | IFNW1 Signature |
| UBE2S | 27338 | IFNW1 Signature |
| UNC93B1 | 81622 | IFNW1 Signature |
| USP18 | 11274 | IFNW1 Signature |
| USP25 | 29761 | IFNW1 Signature |
| WARS | 7453 | IFNW1 Signature |
| WIPF1 | 7456 | IFNW1 Signature |
| WT1 | 7490 | IFNW1 Signature |
| XAF1 | 54739 | IFNW1 Signature |
| ZNF107 | 51427 | IFNW1 Signature |
| ACSL1 | 2180 | TYPE I and TYPE II IFN Core |
| AIM2 | 9447 | TYPE I and TYPE II IFN Core |
| APOL3 | 80833 | TYPE I and TYPE II IFN Core |
| ATF3 | 467 | TYPE I and TYPE II IFN Core |
| CASP1 | 834 | TYPE I and TYPE II IFN Core |
| CASP10 | 843 | TYPE I and TYPE II IFN Core |
| CCL8 | 6355 | TYPE I and TYPE II IFN Core |
| CCND2 | 894 | TYPE I and TYPE II IFN Core |
| CD38 | 952 | TYPE I and TYPE II IFN Core |
| CDKN1A | 1026 | TYPE I and TYPE II IFN Core |
| CFB | 629 | TYPE I and TYPE II IFN Core |
| CXCL10 | 3627 | TYPE I and TYPE II IFN Core |
| CXCL11 | 6373 | TYPE I and TYPE II IFN Core |
| CXCL9 | 4283 | TYPE I and TYPE II IFN Core |
| EDN1 | 1906 | TYPE I and TYPE II IFN Core |
| EPB41 | 2035 | TYPE I and TYPE II IFN Core |
| ETV4 | 2118 | TYPE I and TYPE II IFN Core |
| F8 | 2157 | TYPE I and TYPE II IFN Core |
| FAS | 355 | TYPE I and TYPE II IFN Core |
| GADD45B | 4616 | TYPE I and TYPE II IFN Core |
| GBP1 | 2633 | TYPE I and TYPE II IFN Core |
| GBP2 | 2634 | TYPE I and TYPE II IFN Core |
| GCH1 | 2643 | TYPE I and TYPE II IFN Core |
| GCNT1 | 2650 | TYPE I and TYPE II IFN Core |
| GLS | 2744 | TYPE I and TYPE II IFN Core |
| HBG2 | 3048 | TYPE I and TYPE II IFN Core |
| IDO1 | 3620 | TYPE I and TYPE II IFN Core |
| IFI27 | 3429 | TYPE I and TYPE II IFN Core |
| IFI44 | 10561 | TYPE I and TYPE II IFN Core |
| IL15 | 3600 | TYPE I and TYPE II IFN Core |
| IL15RA | 3601 | TYPE I and TYPE II IFN Core |
| JAK2 | 3717 | TYPE I and TYPE II IFN Core |
| LAP3 | 51056 | TYPE I and TYPE II IFN Core |
| LMNB1 | 4001 | TYPE I and TYPE II IFN Core |
| MRPS15 | 64960 | TYPE I and TYPE II IFN Core |
| MSR1 | 4481 | TYPE I and TYPE II IFN Core |
| NKTR | 4820 | TYPE I and TYPE II IFN Core |
| NR3C1 | 2908 | TYPE I and TYPE II IFN Core |
| OAS1 | 4938 | TYPE I and TYPE II IFN Core |
| OAS3 | 4940 | TYPE I and TYPE II IFN Core |
| PSMB9 | 5698 | TYPE I and TYPE II IFN Core |
| PTCH1 | 5727 | TYPE I and TYPE II IFN Core |
| RGS1 | 5996 | TYPE I and TYPE II IFN Core |
| SERPING1 | 710 | TYPE I and TYPE II IFN Core |
| SOCS1 | 8651 | TYPE I and TYPE II IFN Core |
| SP100 | 6672 | TYPE I and TYPE II IFN Core |
| STAT1 | 6772 | TYPE I and TYPE II IFN Core |
| STAT2 | 6773 | TYPE I and TYPE II IFN Core |
| STX11 | 8676 | TYPE I and TYPE II IFN Core |
| TAP1 | 6890 | TYPE I and TYPE II IFN Core |
| TAP2 | 6891 | TYPE I and TYPE II IFN Core |
| TNFSF10 | 8743 | TYPE I and TYPE II IFN Core |
| UBE2L6 | 9246 | TYPE I and TYPE II IFN Core |
| WARS | 7453 | TYPE I and TYPE II IFN Core |
| ACSL1 | 2180 | Type I IFN Core Signature |
| ADAR | 103 | Type I IFN Core Signature |
| AGT | 183 | Type I IFN Core Signature |
| AIM2 | 9447 | Type I IFN Core Signature |
| AKAP2 | 11217 | Type I IFN Core Signature |
| APOBEC3B | 9582 | Type I IFN Core Signature |
| APOBEC3G | 60489 | Type I IFN Core Signature |
| APOL3 | 80833 | Type I IFN Core Signature |
| ATF3 | 467 | Type I IFN Core Signature |
| ATF5 | 22809 | Type I IFN Core Signature |
| BAG1 | 573 | Type I IFN Core Signature |
| BARD1 | 580 | Type I IFN Core Signature |
| BCL7B | 9275 | Type I IFN Core Signature |
| BLVRA | 644 | Type I IFN Core Signature |
| BRCA1 | 672 | Type I IFN Core Signature |
| BRCA2 | 675 | Type I IFN Core Signature |
| BST2 | 684 | Type I IFN Core Signature |
| CAD | 790 | Type I IFN Core Signature |
| CAMK2A | 815 | Type I IFN Core Signature |
| CASP1 | 834 | Type I IFN Core Signature |
| CASP10 | 843 | Type I IFN Core Signature |
| CASP5 | 838 | Type I IFN Core Signature |
| CBR1 | 873 | Type I IFN Core Signature |
| CBWD1 | 55871 | Type I IFN Core Signature |
| CCL13 | 6357 | Type I IFN Core Signature |
| CCL7 | 6354 | Type I IFN Core Signature |
| CCL8 | 6355 | Type I IFN Core Signature |
| CCNA1 | 8900 | Type I IFN Core Signature |
| CCND2 | 894 | Type I IFN Core Signature |
| CD2AP | 23607 | Type I IFN Core Signature |
| CD38 | 952 | Type I IFN Core Signature |
| CD4 | 920 | Type I IFN Core Signature |
| CD69 | 969 | Type I IFN Core Signature |
| CDKN1A | 1026 | Type I IFN Core Signature |
| CFB | 629 | Type I IFN Core Signature |
| CHKA | 1119 | Type I IFN Core Signature |
| CNTN6 | 27255 | Type I IFN Core Signature |
| COL3A1 | 1281 | Type I IFN Core Signature |
| CTSL | 1514 | Type I IFN Core Signature |
| CXCL10 | 3627 | Type I IFN Core Signature |
| CXCL11 | 6373 | Type I IFN Core Signature |
| CXCL9 | 4283 | Type I IFN Core Signature |
| CXCR2 | 3579 | Type I IFN Core Signature |
| CYP2J2 | 1573 | Type I IFN Core Signature |
| DEFB1 | 1672 | Type I IFN Core Signature |
| DLL1 | 28514 | Type I IFN Core Signature |
| DSC2 | 1824 | Type I IFN Core Signature |
| DUSP5 | 1847 | Type I IFN Core Signature |
| DUSP7 | 1849 | Type I IFN Core Signature |
| DYNLT1 | 6993 | Type I IFN Core Signature |
| DYSF | 8291 | Type I IFN Core Signature |
| ECE1 | 1889 | Type I IFN Core Signature |
| EDN1 | 1906 | Type I IFN Core Signature |
| EIF2AK2 | 5610 | Type I IFN Core Signature |
| EIF2B1 | 1967 | Type I IFN Core Signature |
| EIF4ENIF1 | 56478 | Type I IFN Core Signature |
| ENPP2 | 5168 | Type I IFN Core Signature |
| EPB41 | 2035 | Type I IFN Core Signature |
| ETV4 | 2118 | Type I IFN Core Signature |
| F8 | 2157 | Type I IFN Core Signature |
| FAF1 | 11124 | Type I IFN Core Signature |
| FAS | 355 | Type I IFN Core Signature |
| FASLG | 356 | Type I IFN Core Signature |
| FGF1 | 2246 | Type I IFN Core Signature |
| FLNA | 2316 | Type I IFN Core Signature |
| FOXO1 | 2308 | Type I IFN Core Signature |
| FTL | 2512 | Type I IFN Core Signature |
| FUT4 | 2526 | Type I IFN Core Signature |
| GADD45B | 4616 | Type I IFN Core Signature |
| GBAP1 | 2630 | Type I IFN Core Signature |
| GBP1 | 2633 | Type I IFN Core Signature |
| GBP2 | 2634 | Type I IFN Core Signature |
| GCH1 | 2643 | Type I IFN Core Signature |
| GCNT1 | 2650 | Type I IFN Core Signature |
| GLS | 2744 | Type I IFN Core Signature |
| GMPR | 2766 | Type I IFN Core Signature |
| GPR161 | 23432 | Type I IFN Core Signature |
| GUK1 | 2987 | Type I IFN Core Signature |
| HBG2 | 3048 | Type I IFN Core Signature |
| HIST2H2AA3 | 8337 | Type I IFN Core Signature |
| HLA-DOA | 3111 | Type I IFN Core Signature |
| HS6ST1 | 9394 | Type I IFN Core Signature |
| HSP90AA1 | 3320 | Type I IFN Core Signature |
| IDO1 | 3620 | Type I IFN Core Signature |
| IFI16 | 3428 | Type I IFN Core Signature |
| IFI27 | 3429 | Type I IFN Core Signature |
| IFI35 | 3430 | Type I IFN Core Signature |
| IFI44 | 10561 | Type I IFN Core Signature |
| IFI6 | 2537 | Type I IFN Core Signature |
| IFIT1 | 3434 | Type I IFN Core Signature |
| IFIT5 | 24138 | Type I IFN Core Signature |
| IFITM1 | 8519 | Type I IFN Core Signature |
| IFITM2 | 10581 | Type I IFN Core Signature |
| IFITM3 | 10410 | Type I IFN Core Signature |
| IFRD1 | 3475 | Type I IFN Core Signature |
| IGL | 3535 | Type I IFN Core Signature |
| IKBKG | 8517 | Type I IFN Core Signature |
| IL15 | 3600 | Type I IFN Core Signature |
| IL15RA | 3601 | Type I IFN Core Signature |
| IL1RN | 3557 | Type I IFN Core Signature |
| IL6 | 3569 | Type I IFN Core Signature |
| INPPL1 | 3636 | Type I IFN Core Signature |
| IRF2 | 3660 | Type I IFN Core Signature |
| IRF7 | 3665 | Type I IFN Core Signature |
| ISG15 | 9636 | Type I IFN Core Signature |
| ISG20 | 3669 | Type I IFN Core Signature |
| JAK2 | 3717 | Type I IFN Core Signature |
| JUP | 3728 | Type I IFN Core Signature |
| KCNA3 | 3738 | Type I IFN Core Signature |
| KDELR2 | 11014 | Type I IFN Core Signature |
| KIF20B | 9585 | Type I IFN Core Signature |
| KLF6 | 1316 | Type I IFN Core Signature |
| KPNB1 | 3837 | Type I IFN Core Signature |
| KRT8 | 3856 | Type I IFN Core Signature |
| LAG3 | 3902 | Type I IFN Core Signature |
| LAMP3 | 27074 | Type I IFN Core Signature |
| LAP3 | 51056 | Type I IFN Core Signature |
| LEPR | 3953 | Type I IFN Core Signature |
| LGALS2 | 3957 | Type I IFN Core Signature |
| LGALS3BP | 3959 | Type I IFN Core Signature |
| LGALS9 | 3965 | Type I IFN Core Signature |
| LGMN | 5641 | Type I IFN Core Signature |
| LMNB1 | 4001 | Type I IFN Core Signature |
| LMO2 | 4005 | Type I IFN Core Signature |
| LY6E | 4061 | Type I IFN Core Signature |
| MAP2K5 | 5607 | Type I IFN Core Signature |
| MCL1 | 4170 | Type I IFN Core Signature |
| MED1 | 5469 | Type I IFN Core Signature |
| MGLL | 11343 | Type I IFN Core Signature |
| MNDA | 4332 | Type I IFN Core Signature |
| MRPS15 | 64960 | Type I IFN Core Signature |
| MSR1 | 4481 | Type I IFN Core Signature |
| MX1 | 4599 | Type I IFN Core Signature |
| MX2 | 4600 | Type I IFN Core Signature |
| MYD88 | 4615 | Type I IFN Core Signature |
| NAMPT | 10135 | Type I IFN Core Signature |
| NFE2L3 | 9603 | Type I IFN Core Signature |
| NKTR | 4820 | Type I IFN Core Signature |
| NMI | 9111 | Type I IFN Core Signature |
| NR3C1 | 2908 | Type I IFN Core Signature |
| NUB1 | 51667 | Type I IFN Core Signature |
| NUPR1 | 26471 | Type I IFN Core Signature |
| OAS1 | 4938 | Type I IFN Core Signature |
| OAS2 | 4939 | Type I IFN Core Signature |
| OAS3 | 4940 | Type I IFN Core Signature |
| PATJ | 10207 | Type I IFN Core Signature |
| PDGFB | 5155 | Type I IFN Core Signature |
| PDGFRL | 5157 | Type I IFN Core Signature |
| PKD2 | 5311 | Type I IFN Core Signature |
| PLSCR1 | 5359 | Type I IFN Core Signature |
| PMAIP1 | 5366 | Type I IFN Core Signature |
| PML | 5371 | Type I IFN Core Signature |
| PRKRA | 8575 | Type I IFN Core Signature |
| PSMB9 | 5698 | Type I IFN Core Signature |
| PTCH1 | 5727 | Type I IFN Core Signature |
| RBCK1 | 10616 | Type I IFN Core Signature |
| RGS1 | 5996 | Type I IFN Core Signature |
| RGS6 | 9628 | Type I IFN Core Signature |
| RTP4 | 64108 | Type I IFN Core Signature |
| SAT1 | 6303 | Type I IFN Core Signature |
| SCARB2 | 950 | Type I IFN Core Signature |
| SERPING1 | 710 | Type I IFN Core Signature |
| SIT1 | 27240 | Type I IFN Core Signature |
| SOCS1 | 8651 | Type I IFN Core Signature |
| SP100 | 6672 | Type I IFN Core Signature |
| SP110 | 3431 | Type I IFN Core Signature |
| SP140 | 11262 | Type I IFN Core Signature |
| SPIB | 6689 | Type I IFN Core Signature |
| ST3GAL5 | 8869 | Type I IFN Core Signature |
| STAP1 | 26228 | Type I IFN Core Signature |
| STAT1 | 6772 | Type I IFN Core Signature |
| STAT2 | 6773 | Type I IFN Core Signature |
| STX11 | 8676 | Type I IFN Core Signature |
| SUPT3H | 8464 | Type I IFN Core Signature |
| TAPI | 6890 | Type I IFN Core Signature |
| TAP2 | 6891 | Type I IFN Core Signature |
| TARBP1 | 6894 | Type I IFN Core Signature |
| TCN2 | 6948 | Type I IFN Core Signature |
| TFDP2 | 7029 | Type I IFN Core Signature |
| TGM1 | 7051 | Type I IFN Core Signature |
| TLR3 | 7098 | Type I IFN Core Signature |
| TLR7 | 51284 | Type I IFN Core Signature |
| TNFRSF11A | 8792 | Type I IFN Core Signature |
| TNFSF10 | 8743 | Type I IFN Core Signature |
| TNK2 | 10188 | Type I IFN Core Signature |
| TOR1B | 27348 | Type I IFN Core Signature |
| TRA2B | 6434 | Type I IFN Core Signature |
| TRD | 6964 | Type I IFN Core Signature |
| TRIM21 | 6737 | Type I IFN Core Signature |
| TRIM22 | 10346 | Type I IFN Core Signature |
| TRIM34 | 53840 | Type I IFN Core Signature |
| TRIM38 | 10475 | Type I IFN Core Signature |
| UBA7 | 7318 | Type I IFN Core Signature |
| UBE2L6 | 9246 | Type I IFN Core Signature |
| UBE2S | 27338 | Type I IFN Core Signature |
| UNC93B1 | 81622 | Type I IFN Core Signature |
| USP18 | 11274 | Type I IFN Core Signature |
| WARS | 7453 | Type I IFN Core Signature |
| WT1 | 7490 | Type I IFN Core Signature |
| XAF1 | 54739 | Type I IFN Core Signature |

Table of UCSC Eils Lung Modified

| geneSymbol | geneEntrezID | GeneSet |
|---|---|---|
| ABCA1 | 19 | AT1_Cat |
| ADIRF | 10974 | AT1_Cat |
| AGER | 177 | AT1_Cat |
| ANKRD29 | 147463 | AT1_Cat |
| AQP4 | 361 | AT1_Cat |
| ARAP2 | 116984 | AT1_Cat |
| ARHGEF26 | 26084 | AT1_Cat |
| ATF7IP2 | 80063 | AT1_Cat |
| CAV1 | 857 | AT1_Cat |
| CAV2 | 858 | AT1_Cat |
| CCDC85A | 114800 | AT1_Cat |
| CLDN18 | 51208 | AT1_Cat |
| CLIC5 | 53405 | AT1_Cat |
| CNTN6 | 27255 | AT1_Cat |
| COL4A4 | 1286 | AT1_Cat |
| CYP4B1 | 1580 | AT1_Cat |
| CYR61 | 3491 | AT1_Cat |
| DAPK2 | 23604 | AT1_Cat |
| DOCK4 | 9732 | AT1_Cat |
| EMP2 | 2013 | AT1_Cat |
| EPB41L5 | 57669 | AT1_Cat |
| FAM189A2 | 9413 | AT1_Cat |
| GALNT13 | 114805 | AT1_Cat |
| GPC5 | 2262 | AT1_Cat |
| GPM6A | 2823 | AT1_Cat |
| HOMER1 | 9456 | AT1_Cat |
| KAL1 | 3730 | AT1_Cat |
| KCNT2 | 343450 | AT1_Cat |
| KHDRBS2 | 202559 | AT1_Cat |
| KRT18 | 3875 | AT1_Cat |
| KRT19 | 3880 | AT1_Cat |
| KRT7 | 3855 | AT1_Cat |
| LAMA3 | 3909 | AT1_Cat |
| LIN7A | 8825 | AT1_Cat |
| LINC00842 | 643650 | AT1_Cat |
| MAP2 | 4133 | AT1_Cat |
| MS4A15 | 219995 | AT1_Cat |
| MYO16 | 23026 | AT1_Cat |
| MYO16-AS1 | 100885782 | AT1_Cat |
| NEBL | 10529 | AT1_Cat |
| NTM | 50863 | AT1_Cat |
| PAPSS2 | 9060 | AT1_Cat |
| PHLDB2 | 90102 | AT1_Cat |
| PLCE1 | 51196 | AT1_Cat |
| PTPN21 | 11099 | AT1_Cat |
| RANBP17 | 64901 | AT1_Cat |
| RHOBTB3 | 22836 | AT1_Cat |
| ROR1 | 4919 | AT1_Cat |
| S100A10 | 6281 | AT1_Cat |
| SCD5 | 79966 | AT1_Cat |
| SCEL | 8796 | AT1_Cat |
| SLC1A1 | 6505 | AT1_Cat |
| SLC39A8 | 64116 | AT1_Cat |
| SLC8A1-AS1 | 100128590 | AT1_Cat |
| SPOCK2 | 9806 | AT1_Cat |
| SRPX2 | 27286 | AT1_Cat |
| ST6GALNAC5 | 81849 | AT1_Cat |
| TNFRSF12A | 51330 | AT1_Cat |
| TSPAN13 | 27075 | AT1_Cat |
| VEGFA | 7422 | AT1_Cat |
| ABCA3 | 21 | AT2_Cat |
| ABCC9 | 10060 | AT2_Cat |
| ACOXL | 55289 | AT2_Cat |
| AGBL1 | 123624 | AT2_Cat |
| AGR2 | 10551 | AT2_Cat |
| ALPL | 249 | AT2_Cat |
| ARRDC3 | 57561 | AT2_Cat |
| C3 | 718 | AT2_Cat |
| C8orf34 | 116328 | AT2_Cat |
| C8orf4 | 56892 | AT2_Cat |
| CACHD1 | 57685 | AT2_Cat |
| CCDC141 | 285025 | AT2_Cat |
| COLEC12 | 81035 | AT2_Cat |
| CTSH | 1512 | AT2_Cat |
| DLG2 | 1740 | AT2_Cat |
| DLGAP1 | 9229 | AT2_Cat |
| DMBT1 | 1755 | AT2_Cat |
| ETV1 | 2115 | AT2_Cat |
| ETV5 | 2119 | AT2_Cat |
| FAM155A | 728215 | AT2_Cat |
| FLRT3 | 23767 | AT2_Cat |
| FNIP2 | 57600 | AT2_Cat |
| FREM2 | 341640 | AT2_Cat |
| HHIP | 64399 | AT2_Cat |
| KCNJ15 | 3772 | AT2_Cat |
| KCNQ3 | 3786 | AT2_Cat |
| KIAA1324L | 222223 | AT2_Cat |
| LEPREL1 | 55214 | AT2_Cat |
| LHFPL3 | 375612 | AT2_Cat |
| LINC01090 | 104355152 | AT2_Cat |
| LRP2BP | 55805 | AT2_Cat |
| LRRK2 | 120892 | AT2_Cat |
| MACROD2 | 140733 | AT2_Cat |
| MAPK10 | 5602 | AT2_Cat |
| MFSD2A | 84879 | AT2_Cat |
| NAPSA | 9476 | AT2_Cat |
| NECAB1 | 64168 | AT2_Cat |
| NTN4 | 59277 | AT2_Cat |
| PARM1 | 25849 | AT2_Cat |
| PCDH9 | 5101 | AT2_Cat |
| PCDP1 | 200373 | AT2_Cat |
| PCYOX1 | 51449 | AT2_Cat |
| PGC | 5225 | AT2_Cat |
| PTGFR | 5737 | AT2_Cat |
| PTPN13 | 5783 | AT2_Cat |
| RASGRF1 | 5923 | AT2_Cat |
| RGS16 | 6004 | AT2_Cat |
| RND1 | 27289 | AT2_Cat |
| SCN1A | 6323 | AT2_Cat |
| SDR16C5 | 195814 | AT2_Cat |
| SERPINA1 | 5265 | AT2_Cat |
| SFTPA2 | 729238 | AT2_Cat |
| SFTPC | 6440 | AT2_Cat |
| SFTPD | 6441 | AT2_Cat |
| TMEM163 | 81615 | AT2_Cat |
| TMEM164 | 84187 | AT2_Cat |
| TTN | 7273 | AT2_Cat |
| WIF1 | 11197 | AT2_Cat |
| ZNF385B | 151126 | AT2_Cat |
| ADGB | 79747 | Ciliated |
| AGBL4 | 84871 | Ciliated |
| AK9 | 221264 | Ciliated |
| ANKFN1 | 162282 | Ciliated |
| AQP4-AS1 | 147429 | Ciliated |
| ARMC3 | 219681 | Ciliated |
| C12orf55 | 144535 | Ciliated |
| C20orf26 | 26074 | Ciliated |
| C20orf85 | 128602 | Ciliated |
| C21orf58 | 54058 | Ciliated |
| C4orf47 | 441054 | Ciliated |
| C9orf117 | 286207 | Ciliated |
| C9orf171 | 389799 | Ciliated |
| CAPS | 828 | Ciliated |
| CCDC146 | 57639 | Ciliated |
| CCDC17 | 149483 | Ciliated |
| CCDC170 | 80129 | Ciliated |
| CCDC30 | 728621 | Ciliated |
| CCDC42B | 387885 | Ciliated |
| CCDC78 | 124093 | Ciliated |
| CLEC19A | 728276 | Ciliated |
| DCDC1 | 341019 | Ciliated |
| DNAAF1 | 123872 | Ciliated |
| DNAH11 | 8701 | Ciliated |
| DNAH12 | 201625 | Ciliated |
| DNAH3 | 55567 | Ciliated |
| DNAH6 | 1768 | Ciliated |
| DNAH7 | 56171 | Ciliated |
| DNAI2 | 64446 | Ciliated |
| DPPA2 | 151871 | Ciliated |
| DTHD1 | 401124 | Ciliated |
| DYNC2H1 | 79659 | Ciliated |
| ECT2L | 345930 | Ciliated |
| ESRRG | 2104 | Ciliated |
| FABP6 | 2172 | Ciliated |
| FAM154B | 283726 | Ciliated |
| FHAD1 | 114827 | Ciliated |
| IFLTD1 | 160492 | Ciliated |
| KIAA0825 | 285600 | Ciliated |
| KIAA1377 | 57562 | Ciliated |
| LRGUK | 136332 | Ciliated |
| LRRC48 | 83450 | Ciliated |
| MROH9 | 80133 | Ciliated |
| MS4A8 | 83661 | Ciliated |
| NEK10 | 152110 | Ciliated |
| NEK11 | 79858 | Ciliated |
| NELL2 | 4753 | Ciliated |
| PACRG | 135138 | Ciliated |
| PRMT8 | 56341 | Ciliated |
| PTPRT | 11122 | Ciliated |
| PTRH1 | 138428 | Ciliated |
| RP1 | 6101 | Ciliated |
| RSPH1 | 89765 | Ciliated |
| SLCO2B1 | 11309 | Ciliated |
| SPAG16 | 79582 | Ciliated |
| SPEF2 | 79925 | Ciliated |
| TEKT1 | 83659 | Ciliated |
| TMEM232 | 642987 | Ciliated |
| TPPP3 | 51673 | Ciliated |
| TSPAN19 | 144448 | Ciliated |
| TTC18 | 118491 | Ciliated |
| TTC29 | 83894 | Ciliated |
| ULK4 | 54986 | Ciliated |
| VWA3A | 146177 | Ciliated |
| VWA3B | 200403 | Ciliated |
| WDR78 | 79819 | Ciliated |
| WDR96 | 80217 | Ciliated |
| ZBBX | 79740 | Ciliated |
| ZMYND10 | 51364 | Ciliated |
| AADAC | 13 | Club |
| ADAM28 | 10863 | Club |
| AGR3 | 155465 | Club |
| BMP6 | 654 | Club |
| BPIFB1 | 92747 | Club |
| CASC15 | 401237 | Club |
| CFH | 3075 | Club |
| CHST9 | 83539 | Club |
| CIT | 11113 | Club |
| CLIC6 | 54102 | Club |
| CLU | 1191 | Club |
| CNTN4 | 152330 | Club |
| CP | 1356 | Club |
| CXCL2 | 2920 | Club |
| ERN2 | 10595 | Club |
| EYA2 | 2139 | Club |
| FAM129A | 116496 | Club |
| FGFR2 | 2263 | Club |
| FMO2 | 2327 | Club |
| FOLR1 | 2348 | Club |
| FRAS1 | 80144 | Club |
| GDF15 | 9518 | Club |
| GLIS3 | 169792 | Club |
| HS3ST5 | 222537 | Club |
| KCNQ5 | 56479 | Club |
| KIAA1324 | 57535 | Club |
| KLK10 | 5655 | Club |
| KLK11 | 11012 | Club |
| KRT15 | 3866 | Club |
| LINC00511 | 400619 | Club |
| MAGI2 | 9863 | Club |
| MIR205HG | 642587 | Club |
| MME | 4311 | Club |
| MMP7 | 4316 | Club |
| MT-CO1 | 4512 | Club |
| MT-CO2 | 4513 | Club |
| MT-ND1 | 4535 | Club |
| MT-ND3 | 4537 | Club |
| MUC4 | 4585 | Club |
| NCEH1 | 57552 | Club |
| NHS | 4810 | Club |
| NTN1 | 9423 | Club |
| PIGR | 5284 | Club |
| PLCL1 | 5334 | Club |
| PRSS12 | 8492 | Club |
| SAA1 | 6288 | Club |
| SCGB1A1 | 7356 | Club |
| SOX4 | 6659 | Club |
| SRGAP3 | 9901 | Club |
| TP63 | 8626 | Club |
| VTCN1 | 79679 | Club |
| WFDC2 | 10406 | Club |
| AKT3 | 10000 | Endothelial |
| ANO2 | 57101 | Endothelial |
| AQP1 | 358 | Endothelial |
| BST2 | 684 | Endothelial |
| C10orf10 | 11067 | Endothelial |
| CA4 | 762 | Endothelial |
| CLDN5 | 7122 | Endothelial |
| CLEC14A | 161198 | Endothelial |
| COL4A2 | 1284 | Endothelial |
| CTNNAL1 | 8727 | Endothelial |
| CYYR1 | 116159 | Endothelial |
| DARC | 2532 | Endothelial |
| DPYSL3 | 1809 | Endothelial |
| ELTD1 | 64123 | Endothelial |
| EMP1 | 2012 | Endothelial |
| ENPP2 | 5168 | Endothelial |
| EXOC6 | 54536 | Endothelial |
| FLI1 | 2313 | Endothelial |
| FLT1 | 2321 | Endothelial |
| FOXO1 | 2308 | Endothelial |
| GNG11 | 2791 | Endothelial |
| HLA-E | 3133 | Endothelial |
| ICAM2 | 3384 | Endothelial |
| IGFBP4 | 3487 | Endothelial |
| IL33 | 90865 | Endothelial |
| ITGA1 | 3672 | Endothelial |
| ITM2A | 9452 | Endothelial |
| MEF2C | 4208 | Endothelial |
| MEIS2 | 4212 | Endothelial |
| PALMD | 54873 | Endothelial |
| PLCB1 | 23236 | Endothelial |
| PLCB4 | 5332 | Endothelial |
| PREX2 | 80243 | Endothelial |
| PRSS23 | 11098 | Endothelial |
| PTPRB | 5787 | Endothelial |
| RAMP2 | 10266 | Endothelial |
| SEMA6A | 57556 | Endothelial |
| SH3BP5 | 9467 | Endothelial |
| SLCO2A1 | 6578 | Endothelial |
| SOCS3 | 9021 | Endothelial |
| SPARC | 6678 | Endothelial |
| TCF4 | 6925 | Endothelial |
| TGFBR3 | 7049 | Endothelial |
| THBD | 7056 | Endothelial |
| TM4SF1 | 4071 | Endothelial |
| 3-Mar | 115123 | Lymphatic_Endothelium |
| AKAP12 | 9590 | Lymphatic_Endothelium |
| CCL21 | 6366 | Lymphatic_Endothelium |
| CNKSR3 | 154043 | Lymphatic_Endothelium |
| CNTNAP3B | 728577 | Lymphatic_Endothelium |
| COL8A1 | 1295 | Lymphatic_Endothelium |
| EPB41L3 | 23136 | Lymphatic_Endothelium |
| FABP4 | 2167 | Lymphatic_Endothelium |
| FLRT2 | 23768 | Lymphatic_Endothelium |
| FLT4 | 2324 | Lymphatic_Endothelium |
| GRAPL | 400581 | Lymphatic_Endothelium |
| KALRN | 8997 | Lymphatic_Endothelium |
| KCNIP4 | 80333 | Lymphatic_Endothelium |
| KLHL4 | 56062 | Lymphatic_Endothelium |
| NPAS3 | 64067 | Lymphatic_Endothelium |
| NRP2 | 8828 | Lymphatic_Endothelium |
| PDE1A | 5136 | Lymphatic_Endothelium |
| PDE7B | 27115 | Lymphatic_Endothelium |
| PKHD1L1 | 93035 | Lymphatic_Endothelium |
| PPFIBP1 | 8496 | Lymphatic_Endothelium |
| PROX1 | 5629 | Lymphatic_Endothelium |
| RELN | 5649 | Lymphatic_Endothelium |
| RHOJ | 57381 | Lymphatic_Endothelium |
| SEMA3D | 223117 | Lymphatic_Endothelium |
| SMOC2 | 64094 | Lymphatic_Endothelium |
| SNTG2 | 54221 | Lymphatic_Endothelium |
| ST6GALNAC3 | 256435 | Lymphatic_Endothelium |
| STON2 | 85439 | Lymphatic_Endothelium |
| TFF3 | 7033 | Lymphatic_Endothelium |
| TFPI | 7035 | Lymphatic_Endothelium |
| TSHZ2 | 128553 | Lymphatic_Endothelium |
| TSPAN5 | 10098 | Lymphatic_Endothelium |
| VAV3 | 10451 | Lymphatic_Endothelium |
| ZDHHC14 | 79683 | Lymphatic_Endothelium |
| ZNF521 | 25925 | Lymphatic_Endothelium |

Table of All Myeloid Populations

| geneSymbol | EntrezID | Category |
|---|---|---|
| ANXA1 | 301 | AQP1+ interstitial macrophages steady-state synovium |
| AQP1 | 358 | AQP1+ interstitial macrophages steady-state synovium |
| CD9 | 928 | AQP1+ interstitial macrophages steady-state synovium |
| FXYD2 | 486 | AQP1+ interstitial macrophages steady-state synovium |
| LYVE1 | 10894 | AQP1+ interstitial macrophages steady-state synovium |
| TPPP3 | 51673 | AQP1+ interstitial macrophages steady-state synovium |
| APOE | 348 | CX3CR1+ lining macrophages steady-state synovium |
| CAMK1 | 8536 | CX3CR1+ lining macrophages steady-state synovium |
| CCL3L3 | 414062 | CX3CR1+ lining macrophages steady-state synovium |
| CD83 | 9308 | CX3CR1+ lining macrophages steady-state synovium |
| CTSD | 1509 | CX3CR1+ lining macrophages steady-state synovium |
| FN1 | 2335 | CX3CR1+ lining macrophages steady-state synovium |
| FOLR2 | 2350 | CX3CR1+ lining macrophages steady-state synovium |
| GRN | 2896 | CX3CR1+ lining macrophages steady-state synovium |
| HEXB | 3074 | CX3CR1+ lining macrophages steady-state synovium |
| LTC4S | 4056 | CX3CR1+ lining macrophages steady-state synovium |
| LYZ | 4069 | CX3CR1+ lining macrophages steady-state synovium |
| MAN2B1 | 4125 | CX3CR1+ lining macrophages steady-state synovium |
| NFKBIA | 4792 | CX3CR1+ lining macrophages steady-state synovium |
| NUPR1 | 26471 | CX3CR1+ lining macrophages steady-state synovium |
| PLTP | 5360 | CX3CR1+ lining macrophages steady-state synovium |
| S100B | 6285 | CX3CR1+ lining macrophages steady-state synovium |
| SPARC | 6678 | CX3CR1+ lining macrophages steady-state synovium |
| SRGN | 5552 | CX3CR1+ lining macrophages steady-state synovium |
| SYNGR1 | 9145 | CX3CR1+ lining macrophages steady-state synovium |
| TIMP2 | 7077 | CX3CR1+ lining macrophages steady-state synovium |
| TMEM37 | 140738 | CX3CR1+ lining macrophages steady-state synovium |
| TREM2 | 54209 | CX3CR1+ lining macrophages steady-state synovium |
| VSIG4 | 11326 | CX3CR1+ lining macrophages steady-state synovium |
| CD52 | 1043 | MHCII+ interstitial macrophages steady-state synovium |
| CD74 | 972 | MHCII+ interstitial macrophages steady-state synovium |
| CLEC10A | 10462 | MHCII+ interstitial macrophages steady-state synovium |
| CLEC4A | 50856 | MHCII+ interstitial macrophages steady-state synovium |
| CORO1A | 11151 | MHCII+ interstitial macrophages steady-state synovium |
| GM2A | 2760 | MHCII+ interstitial macrophages steady-state synovium |
| HLA-DMA | 3108 | MHCII+ interstitial macrophages steady-state synovium |
| HLA-DQA1 | 3117 | MHCII+ interstitial macrophages steady-state synovium |
| HLA-DQB1 | 3119 | MHCII+ interstitial macrophages steady-state synovium |
| HLA-DRB5 | 3127 | MHCII+ interstitial macrophages steady-state synovium |
| LSP1 | 4046 | MHCII+ interstitial macrophages steady-state synovium |
| PIM1 | 5292 | MHCII+ interstitial macrophages steady-state synovium |
| CCL13 | 6357 | RELM-α+ interstitial macrophages steady-state synovium |
| CCL7 | 6354 | RELM-α+ interstitial macrophages steady-state synovium |
| CXCL3 | 2921 | RELM-α+ interstitial macrophages steady-state synovium |
| DUSP1 | 1843 | RELM-α+ interstitial macrophages steady-state synovium |
| ZFP36 | 7538 | RELM-α+ interstitial macrophages steady-state synovium |
| JUNB | 3726 | RELM-α+ interstitial macrophages steady-state synovium |
| PF4 | 5196 | RELM-α+ interstitial macrophages steady-state synovium |
| FOS | 2353 | RELM-α+ interstitial macrophages steady-state synovium |
| ATF3 | 467 | RELM-α+ interstitial macrophages steady-state synovium |
| MT1A | 4489 | RELM-α+ interstitial macrophages steady-state synovium |
| IER3 | 8870 | RELM-α+ interstitial macrophages steady-state synovium |
| CCL4 | 6351 | RELM-α+ interstitial macrophages steady-state synovium |
| NFKBIA | 4792 | RELM-α+ interstitial macrophages steady-state synovium |
| CCL24 | 6369 | RELM-α+ interstitial macrophages steady-state synovium |
| MARCKSL1 | 65108 | RELM-α+ interstitial macrophages steady-state synovium |
| CXCL13 | 10563 | RELM-α+ interstitial macrophages steady-state synovium |
| JUN | 3725 | RELM-α+ interstitial macrophages steady-state synovium |
| CCL3L3 | 414062 | RELM-α+ interstitial macrophages steady-state synovium |
| MT2A | 4502 | RELM-α+ interstitial macrophages steady-state synovium |
| KLF6 | 1316 | RELM-α+ interstitial macrophages steady-state synovium |
| PIM1 | 5292 | RELM-α+ interstitial macrophages steady-state synovium |
| IFITM3 | 10410 | RELM-α+ interstitial macrophages steady-state synovium |
| CD83 | 9308 | RELM-α+ interstitial macrophages steady-state synovium |
| BTG2 | 7832 | RELM-α+ interstitial macrophages steady-state synovium |
| KLF4 | 9314 | RELM-α+ interstitial macrophages steady-state synovium |
| HSPA1A | 3303 | RELM-α+ interstitial macrophages steady-state synovium |
| UBC | 7316 | RELM-α+ interstitial macrophages steady-state synovium |
| IER2 | 9592 | RELM-α+ interstitial macrophages steady-state synovium |
| MRC1 | 4360 | RELM-α+ interstitial macrophages steady-state synovium |
| MCL1 | 4170 | RELM-α+ interstitial macrophages steady-state synovium |
| MAF | 4094 | RELM-α+ interstitial macrophages steady-state synovium |
| ANXA1 | 301 | STMN1+ proliferating cells steady-state synovium |
| ANXA2 | 302 | STMN1+ proliferating cells steady-state synovium |
| ARL6IP1 | 23204 | STMN1+ proliferating cells steady-state synovium |
| ATP5IF1 | 93974 | STMN1+ proliferating cells steady-state synovium |
| BIRC5 | 332 | STMN1+ proliferating cells steady-state synovium |
| CBX3 | 11335 | STMN1+ proliferating cells steady-state synovium |
| CD74 | 972 | STMN1+ proliferating cells steady-state synovium |
| GAPDH | 2597 | STMN1+ proliferating cells steady-state synovium |
| H2AFZ | 3015 | STMN1+ proliferating cells steady-state synovium |
| HMGB1 | 3146 | STMN1+ proliferating cells steady-state synovium |
| HMGB2 | 3148 | STMN1+ proliferating cells steady-state synovium |
| HNRNPA3 | 79366 | STMN1+ proliferating cells steady-state synovium |
| JPT1 | 51155 | STMN1+ proliferating cells steady-state synovium |
| LDHA | 3939 | STMN1+ proliferating cells steady-state synovium |
| LGALS1 | 3956 | STMN1+ proliferating cells steady-state synovium |
| LGALS3 | 3958 | STMN1+ proliferating cells steady-state synovium |
| MIF | 4282 | STMN1+ proliferating cells steady-state synovium |
| NCL | 4691 | STMN1+ proliferating cells steady-state synovium |
| NME1 | 4830 | STMN1+ proliferating cells steady-state synovium |
| NPM1 | 4869 | STMN1+ proliferating cells steady-state synovium |
| PKM | 5315 | STMN1+ proliferating cells steady-state synovium |
| PLP2 | 5355 | STMN1+ proliferating cells steady-state synovium |
| PTMA | 5757 | STMN1+ proliferating cells steady-state synovium |
| RAN | 5901 | STMN1+ proliferating cells steady-state synovium |
| RANBP1 | 5902 | STMN1+ proliferating cells steady-state synovium |
| RBM3 | 5935 | STMN1+ proliferating cells steady-state synovium |
| S100A10 | 6281 | STMN1+ proliferating cells steady-state synovium |
| S100A11 | 6282 | STMN1+ proliferating cells steady-state synovium |
| SLC25A5 | 292 | STMN1+ proliferating cells steady-state synovium |
| SNRPE | 6635 | STMN1+ proliferating cells steady-state synovium |
| SNRPG | 6637 | STMN1+ proliferating cells steady-state synovium |
| STMN1 | 3925 | STMN1+ proliferating cells steady-state synovium |
| TAGLN2 | 8407 | STMN1+ proliferating cells steady-state synovium |
| TMSB10 | 9168 | STMN1+ proliferating cells steady-state synovium |
| TUBA1B | 10376 | STMN1+ proliferating cells steady-state synovium |
| TUBA1C | 84790 | STMN1+ proliferating cells steady-state synovium |
| TUBB | 203068 | STMN1+ proliferating cells steady-state synovium |
| TUBB4B | 10383 | STMN1+ proliferating cells steady-state synovium |
| TXN | 7295 | STMN1+ proliferating cells steady-state synovium |
| UBE2C | 11065 | STMN1+ proliferating cells steady-state synovium |
| VIM | 7431 | STMN1+ proliferating cells steady-state synovium |
| ACTB | 60 | CCR2+IL1B+ infiltrating macrophages inflammatory arthritis |
| | | synovium |
| ACTG1 | 71 | CCR2+IL1B+ infiltrating macrophages inflammatory arthritis |
| | | synovium |
| ACTR3 | 10096 | CCR2+IL1B+ infiltrating macrophages inflammatory arthritis |
| | | synovium |
| ADAM8 | 101 | CCR2+IL1B+ infiltrating macrophages inflammatory arthritis |
| | | synovium |
| ADORA2B | 136 | CCR2+IL1B+ infiltrating macrophages inflammatory arthritis |
| | | synovium |
| ADSSL1 | 122622 | CCR2+IL1B+ infiltrating macrophages inflammatory arthritis |
| | | synovium |
| AGPAT4 | 56895 | CCR2+IL1B+ infiltrating macrophages inflammatory arthritis |
| | | synovium |
| ALAS1 | 211 | CCR2+IL1B+ infiltrating macrophages inflammatory arthritis |
| | | synovium |
| ALDOA | 226 | CCR2+IL1B+ infiltrating macrophages inflammatory arthritis |
| | | synovium |
| ALOX5AP | 241 | CCR2+IL1B+ infiltrating macrophages inflammatory arthritis |
| | | synovium |
| ANKRD11 | 29123 | CCR2+IL1B+ infiltrating macrophages inflammatory arthritis |
| | | synovium |
| ANXA2 | 302 | CCR2+IL1B+ infiltrating macrophages inflammatory arthritis |
| | | synovium |
| APRT | 353 | CCR2+IL1B+ infiltrating macrophages inflammatory arthritis |
| | | synovium |
| ARF1 | 375 | CCR2+IL1B+ infiltrating macrophages inflammatory arthritis |
| | | synovium |
| ARF5 | 381 | CCR2+IL1B+ infiltrating macrophages inflammatory arthritis |
| | | synovium |
| ARHGDIB | 397 | CCR2+IL1B+ infiltrating macrophages inflammatory arthritis |
| | | synovium |
| ARPC1B | 10095 | CCR2+IL1B+ infiltrating macrophages inflammatory arthritis |
| | | synovium |
| ARPC2 | 10109 | CCR2+IL1B+ infiltrating macrophages inflammatory arthritis |
| | | synovium |
| ARPC3 | 10094 | CCR2+IL1B+ infiltrating macrophages inflammatory arthritis |
| | | synovium |
| ARPC4 | 10093 | CCR2+IL1B+ infiltrating macrophages inflammatory arthritis |
| | | synovium |
| ATF3 | 467 | CCR2+IL1B+ infiltrating macrophages inflammatory arthritis |
| | | synovium |
| ATP5C1 | 509 | CCR2+IL1B+ infiltrating macrophages inflammatory arthritis |
| | | synovium |
| ATP5F1 | 515 | CCR2+IL1B+ infiltrating macrophages inflammatory arthritis |
| | | synovium |
| B4GALNT1 | 2583 | CCR2+IL1B+ infiltrating macrophages inflammatory arthritis |
| | | synovium |
| BAK1 | 578 | CCR2+IL1B+ infiltrating macrophages inflammatory arthritis |
| | | synovium |
| BIN2 | 51411 | CCR2+IL1B+ infiltrating macrophages inflammatory arthritis |
| | | synovium |
| BIRC3 | 330 | CCR2+IL1B+ infiltrating macrophages inflammatory arthritis |
| | | synovium |
| BTF3 | 689 | CCR2+IL1B+ infiltrating macrophages inflammatory arthritis |
| | | synovium |
| BTG1 | 694 | CCR2+IL1B+ infiltrating macrophages inflammatory arthritis |
| | | synovium |
| BTG2 | 7832 | CCR2+IL1B+ infiltrating macrophages inflammatory arthritis |
| | | synovium |
| C19orf38 | 255809 | CCR2+IL1B+ infiltrating macrophages inflammatory arthritis |
| | | synovium |
| C19orf53 | 28974 | CCR2+IL1B+ infiltrating macrophages inflammatory arthritis |
| | | synovium |
| C5AR1 | 728 | CCR2+IL1B+ infiltrating macrophages inflammatory arthritis |
| | | synovium |
| C5orf30 | 90355 | CCR2+IL1B+ infiltrating macrophages inflammatory arthritis |
| | | synovium |
| CAPZA2 | 830 | CCR2+IL1B+ infiltrating macrophages inflammatory arthritis |
| | | synovium |
| CAPZB | 832 | CCR2+IL1B+ infiltrating macrophages inflammatory arthritis |
| | | synovium |
| CASP6 | 839 | CCR2+IL1B+ infiltrating macrophages inflammatory arthritis |
| | | synovium |
| CCDC12 | 151903 | CCR2+IL1B+ infiltrating macrophages inflammatory arthritis |
| | | synovium |
| CCL13 | 6357 | CCR2+IL1B+ infiltrating macrophages inflammatory arthritis |
| | | synovium |
| CCL3L3 | 414062 | CCR2+IL1B+ infiltrating macrophages inflammatory arthritis |
| | | synovium |
| CCND3 | 896 | CCR2+IL1B+ infiltrating macrophages inflammatory arthritis |
| | | synovium |
| CCNL1 | 57018 | CCR2+IL1B+ infiltrating macrophages inflammatory arthritis |
| | | synovium |
| CCR1 | 1230 | CCR2+IL1B+ infiltrating macrophages inflammatory arthritis |
| | | synovium |
| CCR2 | 729230 | CCR2+IL1B+ infiltrating macrophages inflammatory arthritis |
| | | synovium |
| CCRL2 | 9034 | CCR2+IL1B+ infiltrating macrophages inflammatory arthritis |
| | | synovium |
| CD14 | 929 | CCR2+IL1B+ infiltrating macrophages inflammatory arthritis |
| | | synovium |
| CD52 | 1043 | CCR2+IL1B+ infiltrating macrophages inflammatory arthritis |
| | | synovium |
| CD53 | 963 | CCR2+IL1B+ infiltrating macrophages inflammatory arthritis |
| | | synovium |
| CD86 | 942 | CCR2+IL1B+ infiltrating macrophages inflammatory arthritis |
| | | synovium |
| CDK2AP2 | 10263 | CCR2+IL1B+ infiltrating macrophages inflammatory arthritis |
| | | synovium |
| CDKN1A | 1026 | CCR2+IL1B+ infiltrating macrophages inflammatory arthritis |
| | | synovium |
| CDKN2D | 1032 | CCR2+IL1B+ infiltrating macrophages inflammatory arthritis |
| | | synovium |
| CFP | 5199 | CCR2+IL1B+ infiltrating macrophages inflammatory arthritis |
| | | synovium |
| CHD7 | 55636 | CCR2+IL1B+ infiltrating macrophages inflammatory arthritis |
| | | synovium |
| CHMP4B | 128866 | CCR2+IL1B+ infiltrating macrophages inflammatory arthritis |
| | | synovium |
| CISD2 | 493856 | CCR2+IL1B+ infiltrating macrophages inflammatory arthritis |
| | | synovium |
| CLEC4A | 50856 | CCR2+IL1B+ infiltrating macrophages inflammatory arthritis |
| | | synovium |
| CLEC4D | 338339 | CCR2+IL1B+ infiltrating macrophages inflammatory arthritis |
| | | synovium |
| CLEC4E | 26253 | CCR2+IL1B+ infiltrating macrophages inflammatory arthritis |
| | | synovium |
| CLEC6A | 93978 | CCR2+IL1B+ infiltrating macrophages inflammatory arthritis |
| | | synovium |
| CLIC1 | 1192 | CCR2+IL1B+ infiltrating macrophages inflammatory arthritis |
| | | synovium |
| CNN2 | 1265 | CCR2+IL1B+ infiltrating macrophages inflammatory arthritis |
| | | synovium |
| COPE | 11316 | CCR2+IL1B+ infiltrating macrophages inflammatory arthritis |
| | | synovium |
| CORO1A | 11151 | CCR2+IL1B+ infiltrating macrophages inflammatory arthritis |
| | | synovium |
| CORO1B | 57175 | CCR2+IL1B+ infiltrating macrophages inflammatory arthritis |
| | | synovium |
| COTL1 | 23406 | CCR2+IL1B+ infiltrating macrophages inflammatory arthritis |
| | | synovium |
| CSF2RB | 1439 | CCR2+IL1B+ infiltrating macrophages inflammatory arthritis |
| | | synovium |
| CTSC | 1075 | CCR2+IL1B+ infiltrating macrophages inflammatory arthritis |
| | | synovium |
| CTSH | 1512 | CCR2+IL1B+ infiltrating macrophages inflammatory arthritis |
| | | synovium |
| CXCL3 | 2921 | CCR2+IL1B+ infiltrating macrophages inflammatory arthritis |
| | | synovium |
| CYP4F2 | 8529 | CCR2+IL1B+ infiltrating macrophages inflammatory arthritis |
| | | synovium |
| CYTIP | 9595 | CCR2+IL1B+ infiltrating macrophages inflammatory arthritis |
| | | synovium |
| DDX39A | 10212 | CCR2+IL1B+ infiltrating macrophages inflammatory arthritis |
| | | synovium |
| DENND4A | 10260 | CCR2+IL1B+ infiltrating macrophages inflammatory arthritis |
| | | synovium |
| DMKN | 93099 | CCR2+IL1B+ infiltrating macrophages inflammatory arthritis |
| | | synovium |
| DUSP1 | 1843 | CCR2+IL1B+ infiltrating macrophages inflammatory arthritis |
| | | synovium |
| EIF3F | 8665 | CCR2+IL1B+ infiltrating macrophages inflammatory arthritis |
| | | synovium |
| EIF3H | 8667 | CCR2+IL1B+ infiltrating macrophages inflammatory arthritis |
| | | synovium |
| EIF3K | 27335 | CCR2+IL1B+ infiltrating macrophages inflammatory arthritis |
| | | synovium |
| EIF4A1 | 1973 | CCR2+IL1B+ infiltrating macrophages inflammatory arthritis |
| | | synovium |
| EIF5A | 1984 | CCR2+IL1B+ infiltrating macrophages inflammatory arthritis |
| | | synovium |
| EMB | 133418 | CCR2+IL1B+ infiltrating macrophages inflammatory arthritis |
| | | synovium |
| EMD | 2010 | CCR2+IL1B+ infiltrating macrophages inflammatory arthritis |
| | | synovium |
| EMILIN2 | 84034 | CCR2+IL1B+ infiltrating macrophages inflammatory arthritis |
| | | synovium |
| ENO1 | 2023 | CCR2+IL1B+ infiltrating macrophages inflammatory arthritis |
| | | synovium |
| ERP44 | 23071 | CCR2+IL1B+ infiltrating macrophages inflammatory arthritis |
| | | synovium |
| ESD | 2098 | CCR2+IL1B+ infiltrating macrophages inflammatory arthritis |
| | | synovium |
| EVL | 51466 | CCR2+IL1B+ infiltrating macrophages inflammatory arthritis |
| | | synovium |
| F10 | 2159 | CCR2+IL1B+ infiltrating macrophages inflammatory arthritis |
| | | synovium |
| FAM107B | 83641 | CCR2+IL1B+ infiltrating macrophages inflammatory arthritis |
| | | synovium |
| FAM49B | 51571 | CCR2+IL1B+ infiltrating macrophages inflammatory arthritis |
| | | synovium |
| FAM96A | 84191 | CCR2+IL1B+ infiltrating macrophages inflammatory arthritis |
| | | synovium |
| FAU | 2197 | CCR2+IL1B+ infiltrating macrophages inflammatory arthritis |
| | | synovium |
| FCER1G | 2207 | CCR2+IL1B+ infiltrating macrophages inflammatory arthritis |
| | | synovium |
| FCGR1A | 2209 | CCR2+IL1B+ infiltrating macrophages inflammatory arthritis |
| | | synovium |
| FERMT3 | 83706 | CCR2+IL1B+ infiltrating macrophages inflammatory arthritis |
| | | synovium |
| FES | 2242 | CCR2+IL1B+ infiltrating macrophages inflammatory arthritis |
| | | synovium |
| FGR | 2268 | CCR2+IL1B+ infiltrating macrophages inflammatory arthritis |
| | | synovium |
| FIS1 | 51024 | CCR2+IL1B+ infiltrating macrophages inflammatory arthritis |
| | | synovium |
| FLOT1 | 10211 | CCR2+IL1B+ infiltrating macrophages inflammatory arthritis |
| | | synovium |
| FOS | 2353 | CCR2+IL1B+ infiltrating macrophages inflammatory arthritis |
| | | synovium |
| FOSL2 | 2355 | CCR2+IL1B+ infiltrating macrophages inflammatory arthritis |
| | | synovium |
| FXYD5 | 53827 | CCR2+IL1B+ infiltrating macrophages inflammatory arthritis |
| | | synovium |
| FYB | 2533 | CCR2+IL1B+ infiltrating macrophages inflammatory arthritis |
| | | synovium |
| GADD45B | 4616 | CCR2+IL1B+ infiltrating macrophages inflammatory arthritis |
| | | synovium |
| GADD45G | 10912 | CCR2+IL1B+ infiltrating macrophages inflammatory arthritis |
| | | synovium |
| GAPDH | 2597 | CCR2+IL1B+ infiltrating macrophages inflammatory arthritis |
| | | synovium |
| GCSH | 2653 | CCR2+IL1B+ infiltrating macrophages inflammatory arthritis |
| | | synovium |
| GDA | 9615 | CCR2+IL1B+ infiltrating macrophages inflammatory arthritis |
| | | synovium |
| GDI2 | 2665 | CCR2+IL1B+ infiltrating macrophages inflammatory arthritis |
| | | synovium |
| GLIPR2 | 152007 | CCR2+IL1B+ infiltrating macrophages inflammatory arthritis |
| | | synovium |
| GLRX | 2745 | CCR2+IL1B+ infiltrating macrophages inflammatory arthritis |
| | | synovium |
| GM2A | 2760 | CCR2+IL1B+ infiltrating macrophages inflammatory arthritis |
| | | synovium |
| GMFG | 9535 | CCR2+IL1B+ infiltrating macrophages inflammatory arthritis |
| | | synovium |
| GNB2 | 2783 | CCR2+IL1B+ infiltrating macrophages inflammatory arthritis |
| | | synovium |
| GNGT2 | 2793 | CCR2+IL1B+ infiltrating macrophages inflammatory arthritis |
| | | synovium |
| GPR132 | 29933 | CCR2+IL1B+ infiltrating macrophages inflammatory arthritis |
| | | synovium |
| GSDMD | 79792 | CCR2+IL1B+ infiltrating macrophages inflammatory arthritis |
| | | synovium |
| GSR | 2936 | CCR2+IL1B+ infiltrating macrophages inflammatory arthritis |
| | | synovium |
| H2AFJ | 55766 | CCR2+IL1B+ infiltrating macrophages inflammatory arthritis |
| | | synovium |
| H2AFY | 9555 | CCR2+IL1B+ infiltrating macrophages inflammatory arthritis |
| | | synovium |
| H3F3A | 3020 | CCR2+IL1B+ infiltrating macrophages inflammatory arthritis |
| | | synovium |
| HCK | 3055 | CCR2+IL1B+ infiltrating macrophages inflammatory arthritis |
| | | synovium |
| HCLS1 | 3059 | CCR2+IL1B+ infiltrating macrophages inflammatory arthritis |
| | | synovium |
| HEBP1 | 50865 | CCR2+IL1B+ infiltrating macrophages inflammatory arthritis |
| | | synovium |
| HIF1A | 3091 | CCR2+IL1B+ infiltrating macrophages inflammatory arthritis |
| | | synovium |
| HLA-DMA | 3108 | CCR2+IL1B+ infiltrating macrophages inflammatory arthritis |
| | | synovium |
| HLA-DMB | 3109 | CCR2+IL1B+ infiltrating macrophages inflammatory arthritis |
| | | synovium |
| HM13 | 81502 | CCR2+IL1B+ infiltrating macrophages inflammatory arthritis |
| | | synovium |
| HMGCL | 3155 | CCR2+IL1B+ infiltrating macrophages inflammatory arthritis |
| | | synovium |
| HNRNPA3 | 220988 | CCR2+IL1B+ infiltrating macrophages inflammatory arthritis |
| | | synovium |
| HNRNPDL | 9987 | CCR2+IL1B+ infiltrating macrophages inflammatory arthritis |
| | | synovium |
| HNRNPK | 3190 | CCR2+IL1B+ infiltrating macrophages inflammatory arthritis |
| | | synovium |
| HP | 3240 | CCR2+IL1B+ infiltrating macrophages inflammatory arthritis |
| | | synovium |
| HPCAL1 | 3241 | CCR2+IL1B+ infiltrating macrophages inflammatory arthritis |
| | | synovium |
| HSPA5 | 3309 | CCR2+IL1B+ infiltrating macrophages inflammatory arthritis |
| | | synovium |
| HSPA8 | 3312 | CCR2+IL1B+ infiltrating macrophages inflammatory arthritis |
| | | synovium |
| ID2 | 3398 | CCR2+IL1B+ infiltrating macrophages inflammatory arthritis |
| | | synovium |
| IER3 | 8870 | CCR2+IL1B+ infiltrating macrophages inflammatory arthritis |
| | | synovium |
| IFI16 | 3428 | CCR2+IL1B+ infiltrating macrophages inflammatory arthritis |
| | | synovium |
| IFITM2 | 10581 | CCR2+IL1B+ infiltrating macrophages inflammatory arthritis |
| | | synovium |
| IFITM3 | 10410 | CCR2+IL1B+ infiltrating macrophages inflammatory arthritis |
| | | synovium |
| IFNAR2 | 3455 | CCR2+IL1B+ infiltrating macrophages inflammatory arthritis |
| | | synovium |
| IFNGR1 | 3459 | CCR2+IL1B+ infiltrating macrophages inflammatory arthritis |
| | | synovium |
| IFNGR2 | 3460 | CCR2+IL1B+ infiltrating macrophages inflammatory arthritis |
| | | synovium |
| IFRD1 | 3475 | CCR2+IL1B+ infiltrating macrophages inflammatory arthritis |
| | | synovium |
| IGSF6 | 10261 | CCR2+IL1B+ infiltrating macrophages inflammatory arthritis |
| | | synovium |
| IL17RA | 23765 | CCR2+IL1B+ infiltrating macrophages inflammatory arthritis |
| | | synovium |
| IL18 | 3606 | CCR2+IL1B+ infiltrating macrophages inflammatory arthritis |
| | | synovium |
| IL1B | 3553 | CCR2+IL1B+ infiltrating macrophages inflammatory arthritis |
| | | synovium |
| IL1RN | 3557 | CCR2+IL1B+ infiltrating macrophages inflammatory arthritis |
| | | synovium |
| IRF5 | 3663 | CCR2+IL1B+ infiltrating macrophages inflammatory arthritis |
| | | synovium |
| ISCU | 23479 | CCR2+IL1B+ infiltrating macrophages inflammatory arthritis |
| | | synovium |
| ITGB2 | 3689 | CCR2+IL1B+ infiltrating macrophages inflammatory arthritis |
| | | synovium |
| ITGB7 | 3695 | CCR2+IL1B+ infiltrating macrophages inflammatory arthritis |
| | | synovium |
| JARID2 | 3720 | CCR2+IL1B+ infiltrating macrophages inflammatory arthritis |
| | | synovium |
| JUN | 3725 | CCR2+IL1B+ infiltrating macrophages inflammatory arthritis |
| | | synovium |
| JUNB | 3726 | CCR2+IL1B+ infiltrating macrophages inflammatory arthritis |
| | | synovium |
| KDM6B | 23135 | CCR2+IL1B+ infiltrating macrophages inflammatory arthritis |
| | | synovium |
| KDM7A | 80853 | CCR2+IL1B+ infiltrating macrophages inflammatory arthritis |
| | | synovium |
| LDHA | 3939 | CCR2+IL1B+ infiltrating macrophages inflammatory arthritis |
| | | synovium |
| LGALS3 | 3958 | CCR2+IL1B+ infiltrating macrophages inflammatory arthritis |
| | | synovium |
| LILRB3 | 11025 | CCR2+IL1B+ infiltrating macrophages inflammatory arthritis |
| | | synovium |
| LIMD2 | 80774 | CCR2+IL1B+ infiltrating macrophages inflammatory arthritis |
| | | synovium |
| LITAF | 9516 | CCR2+IL1B+ infiltrating macrophages inflammatory arthritis |
| | | synovium |
| LMAN2 | 10960 | CCR2+IL1B+ infiltrating macrophages inflammatory arthritis |
| | | synovium |
| LMNB1 | 4001 | CCR2+IL1B+ infiltrating macrophages inflammatory arthritis |
| | | synovium |
| LRP10 | 26020 | CCR2+IL1B+ infiltrating macrophages inflammatory arthritis |
| | | synovium |
| LRRC25 | 126364 | CCR2+IL1B+ infiltrating macrophages inflammatory arthritis |
| | | synovium |
| LRRFIP1 | 9208 | CCR2+IL1B+ infiltrating macrophages inflammatory arthritis |
| | | synovium |
| LSP1 | 4046 | CCR2+IL1B+ infiltrating macrophages inflammatory arthritis |
| | | synovium |
| LTB4R | 1241 | CCR2+IL1B+ infiltrating macrophages inflammatory arthritis |
| | | synovium |
| LY6E | 4061 | CCR2+IL1B+ infiltrating macrophages inflammatory arthritis |
| | | synovium |
| LYN | 4067 | CCR2+IL1B+ infiltrating macrophages inflammatory arthritis |
| | | synovium |
| MANF | 7873 | CCR2+IL1B+ infiltrating macrophages inflammatory arthritis |
| | | synovium |
| MAP2K3 | 5606 | CCR2+IL1B+ infiltrating macrophages inflammatory arthritis |
| | | synovium |
| MCUB | 55013 | CCR2+IL1B+ infiltrating macrophages inflammatory arthritis |
| | | synovium |
| MEFV | 4210 | CCR2+IL1B+ infiltrating macrophages inflammatory arthritis |
| | | synovium |
| MGST1 | 4257 | CCR2+IL1B+ infiltrating macrophages inflammatory arthritis |
| | | synovium |
| MILR1 | 284021 | CCR2+IL1B+ infiltrating macrophages inflammatory arthritis |
| | | synovium |
| MMP19 | 4327 | CCR2+IL1B+ infiltrating macrophages inflammatory arthritis |
| | | synovium |
| MPEG1 | 219972 | CCR2+IL1B+ infiltrating macrophages inflammatory arthritis |
| | | synovium |
| MRPL33 | 9553 | CCR2+IL1B+ infiltrating macrophages inflammatory arthritis |
| | | synovium |
| MRPL52 | 122704 | CCR2+IL1B+ infiltrating macrophages inflammatory arthritis |
| | | synovium |
| MSR1 | 4481 | CCR2+IL1B+ infiltrating macrophages inflammatory arthritis |
| | | synovium |
| MSRB1 | 51734 | CCR2+IL1B+ infiltrating macrophages inflammatory arthritis |
| | | synovium |
| MXD1 | 4084 | CCR2+IL1B+ infiltrating macrophages inflammatory arthritis |
| | | synovium |
| MYD88 | 4615 | CCR2+IL1B+ infiltrating macrophages inflammatory arthritis |
| | | synovium |
| MYL12A | 10627 | CCR2+IL1B+ infiltrating macrophages inflammatory arthritis |
| | | synovium |
| MYL12B | 103910 | CCR2+IL1B+ infiltrating macrophages inflammatory arthritis |
| | | synovium |
| NAPSA | 9476 | CCR2+IL1B+ infiltrating macrophages inflammatory arthritis |
| | | synovium |
| NCF2 | 4688 | CCR2+IL1B+ infiltrating macrophages inflammatory arthritis |
| | | synovium |
| NCF4 | 4689 | CCR2+IL1B+ infiltrating macrophages inflammatory arthritis |
| | | synovium |
| NDEL1 | 81565 | CCR2+IL1B+ infiltrating macrophages inflammatory arthritis |
| | | synovium |
| NDUFA3 | 4696 | CCR2+IL1B+ infiltrating macrophages inflammatory arthritis |
| | | synovium |
| NFE2L2 | 4780 | CCR2+IL1B+ infiltrating macrophages inflammatory arthritis |
| | | synovium |
| NFKBIA | 4792 | CCR2+IL1B+ infiltrating macrophages inflammatory arthritis |
| | | synovium |
| NFKBIB | 4793 | CCR2+IL1B+ infiltrating macrophages inflammatory arthritis |
| | | synovium |
| NFKBIZ | 64332 | CCR2+IL1B+ infiltrating macrophages inflammatory arthritis |
| | | synovium |
| NMI | 9111 | CCR2+IL1B+ infiltrating macrophages inflammatory arthritis |
| | | synovium |
| NOP53 | 29997 | CCR2+IL1B+ infiltrating macrophages inflammatory arthritis |
| | | synovium |
| NR4A1 | 3164 | CCR2+IL1B+ infiltrating macrophages inflammatory arthritis |
| | | synovium |
| NR4A2 | 4929 | CCR2+IL1B+ infiltrating macrophages inflammatory arthritis |
| | | synovium |
| NRROS | 375387 | CCR2+IL1B+ infiltrating macrophages inflammatory arthritis |
| | | synovium |
| OSM | 5008 | CCR2+IL1B+ infiltrating macrophages inflammatory arthritis |
| | | synovium |
| PDE4B | 5142 | CCR2+IL1B+ infiltrating macrophages inflammatory arthritis |
| | | synovium |
| PDIA6 | 10130 | CCR2+IL1B+ infiltrating macrophages inflammatory arthritis |
| | | synovium |
| PGK1 | 5230 | CCR2+IL1B+ infiltrating macrophages inflammatory arthritis |
| | | synovium |
| PHLDA1 | 22822 | CCR2+IL1B+ infiltrating macrophages inflammatory arthritis |
| | | synovium |
| PICALM | 8301 | CCR2+IL1B+ infiltrating macrophages inflammatory arthritis |
| | | synovium |
| PID1 | 55022 | CCR2+IL1B+ infiltrating macrophages inflammatory arthritis |
| | | synovium |
| PIM1 | 5292 | CCR2+IL1B+ infiltrating macrophages inflammatory arthritis |
| | | synovium |
| PITPNA | 5306 | CCR2+IL1B+ infiltrating macrophages inflammatory arthritis |
| | | synovium |
| PKM | 5315 | CCR2+IL1B+ infiltrating macrophages inflammatory arthritis |
| | | synovium |
| PLA2G7 | 7941 | CCR2+IL1B+ infiltrating macrophages inflammatory arthritis |
| | | synovium |
| PLAC8 | 51316 | CCR2+IL1B+ infiltrating macrophages inflammatory arthritis |
| | | synovium |
| PLAUR | 5329 | CCR2+IL1B+ infiltrating macrophages inflammatory arthritis |
| | | synovium |
| PLBD1 | 79887 | CCR2+IL1B+ infiltrating macrophages inflammatory arthritis |
| | | synovium |
| PLD4 | 122618 | CCR2+IL1B+ infiltrating macrophages inflammatory arthritis |
| | | synovium |
| PLEK | 5341 | CCR2+IL1B+ infiltrating macrophages inflammatory arthritis |
| | | synovium |
| PLIN2 | 123 | CCR2+IL1B+ infiltrating macrophages inflammatory arthritis |
| | | synovium |
| POLD4 | 57804 | CCR2+IL1B+ infiltrating macrophages inflammatory arthritis |
| | | synovium |
| POR | 5447 | CCR2+IL1B+ infiltrating macrophages inflammatory arthritis |
| | | synovium |
| PPP1CA | 5499 | CCR2+IL1B+ infiltrating macrophages inflammatory arthritis |
| | | synovium |
| PPP1R15A | 23645 | CCR2+IL1B+ infiltrating macrophages inflammatory arthritis |
| | | synovium |
| PPP4C | 5531 | CCR2+IL1B+ infiltrating macrophages inflammatory arthritis |
| | | synovium |
| PPT1 | 5538 | CCR2+IL1B+ infiltrating macrophages inflammatory arthritis |
| | | synovium |
| PRDX5 | 25824 | CCR2+IL1B+ infiltrating macrophages inflammatory arthritis |
| | | synovium |
| PRDX6 | 9588 | CCR2+IL1B+ infiltrating macrophages inflammatory arthritis |
| | | synovium |
| PSMA1 | 5682 | CCR2+IL1B+ infiltrating macrophages inflammatory arthritis |
| | | synovium |
| PSMA7 | 5688 | CCR2+IL1B+ infiltrating macrophages inflammatory arthritis |
| | | synovium |
| PSMB10 | 5699 | CCR2+IL1B+ infiltrating macrophages inflammatory arthritis |
| | | synovium |
| PSMB8 | 5696 | CCR2+IL1B+ infiltrating macrophages inflammatory arthritis |
| | | synovium |
| PSMB9 | 5698 | CCR2+IL1B+ infiltrating macrophages inflammatory arthritis |
| | | synovium |
| PSME1 | 5720 | CCR2+IL1B+ infiltrating macrophages inflammatory arthritis |
| | | synovium |
| PSME2 | 5721 | CCR2+IL1B+ infiltrating macrophages inflammatory arthritis |
| | | synovium |
| PSTPIP1 | 9051 | CCR2+IL1B+ infiltrating macrophages inflammatory arthritis |
| | | synovium |
| PTPN1 | 5770 | CCR2+IL1B+ infiltrating macrophages inflammatory arthritis |
| | | synovium |
| PTPN6 | 5777 | CCR2+IL1B+ infiltrating macrophages inflammatory arthritis |
| | | synovium |
| PTPRC | 5788 | CCR2+IL1B+ infiltrating macrophages inflammatory arthritis |
| | | synovium |
| PXK | 54899 | CCR2+IL1B+ infiltrating macrophages inflammatory arthritis |
| | | synovium |
| PYCARD | 29108 | CCR2+IL1B+ infiltrating macrophages inflammatory arthritis |
| | | synovium |
| RAB32 | 10981 | CCR2+IL1B+ infiltrating macrophages inflammatory arthritis |
| | | synovium |
| RAC2 | 5880 | CCR2+IL1B+ infiltrating macrophages inflammatory arthritis |
| | | synovium |
| RBM3 | 5935 | CCR2+IL1B+ infiltrating macrophages inflammatory arthritis |
| | | synovium |
| RBM39 | 9584 | CCR2+IL1B+ infiltrating macrophages inflammatory arthritis |
| | | synovium |
| RGCC | 28984 | CCR2+IL1B+ infiltrating macrophages inflammatory arthritis |
| | | synovium |
| RGS2 | 5997 | CCR2+IL1B+ infiltrating macrophages inflammatory arthritis |
| | | synovium |
| RHOG | 391 | CCR2+IL1B+ infiltrating macrophages inflammatory arthritis |
| | | synovium |
| RNF149 | 284996 | CCR2+IL1B+ infiltrating macrophages inflammatory arthritis |
| | | synovium |
| RNH1 | 6050 | CCR2+IL1B+ infiltrating macrophages inflammatory arthritis |
| | | synovium |
| RNPEP | 6051 | CCR2+IL1B+ infiltrating macrophages inflammatory arthritis |
| | | synovium |
| RPL10 | 6134 | CCR2+IL1B+ infiltrating macrophages inflammatory arthritis |
| | | synovium |
| RPL13 | 6137 | CCR2+IL1B+ infiltrating macrophages inflammatory arthritis |
| | | synovium |
| RPL15 | 6138 | CCR2+IL1B+ infiltrating macrophages inflammatory arthritis |
| | | synovium |
| RPL17 | 6139 | CCR2+IL1B+ infiltrating macrophages inflammatory arthritis |
| | | synovium |
| RPL18 | 6141 | CCR2+IL1B+ infiltrating macrophages inflammatory arthritis |
| | | synovium |
| RPL18A | 6142 | CCR2+IL1B+ infiltrating macrophages inflammatory arthritis |
| | | synovium |
| RPL19 | 6143 | CCR2+IL1B+ infiltrating macrophages inflammatory arthritis |
| | | synovium |
| RPL24 | 6152 | CCR2+IL1B+ infiltrating macrophages inflammatory arthritis |
| | | synovium |
| RPL27 | 6155 | CCR2+IL1B+ infiltrating macrophages inflammatory arthritis |
| | | synovium |
| RPL27A | 6157 | CCR2+IL1B+ infiltrating macrophages inflammatory arthritis |
| | | synovium |
| RPL34 | 6164 | CCR2+IL1B+ infiltrating macrophages inflammatory arthritis |
| | | synovium |
| RPL36AL | 6166 | CCR2+IL1B+ infiltrating macrophages inflammatory arthritis |
| | | synovium |
| RPL4 | 6124 | CCR2+IL1B+ infiltrating macrophages inflammatory arthritis |
| | | synovium |
| RPL8 | 6132 | CCR2+IL1B+ infiltrating macrophages inflammatory arthritis |
| | | synovium |
| RPL9 | 6133 | CCR2+IL1B+ infiltrating macrophages inflammatory arthritis |
| | | synovium |
| RPLP0 | 6175 | CCR2+IL1B+ infiltrating macrophages inflammatory arthritis |
| | | synovium |
| RPS14 | 6208 | CCR2+IL1B+ infiltrating macrophages inflammatory arthritis |
| | | synovium |
| RPS15A | 6210 | CCR2+IL1B+ infiltrating macrophages inflammatory arthritis |
| | | synovium |
| RPS16 | 6217 | CCR2+IL1B+ infiltrating macrophages inflammatory arthritis |
| | | synovium |
| RPS18 | 6222 | CCR2+IL1B+ infiltrating macrophages inflammatory arthritis |
| | | synovium |
| RPS19 | 6223 | CCR2+IL1B+ infiltrating macrophages inflammatory arthritis |
| | | synovium |
| RPS27 | 6232 | CCR2+IL1B+ infiltrating macrophages inflammatory arthritis |
| | | synovium |
| RPS27A | 6233 | CCR2+IL1B+ infiltrating macrophages inflammatory arthritis |
| | | synovium |
| RPS3 | 6188 | CCR2+IL1B+ infiltrating macrophages inflammatory arthritis |
| | | synovium |
| RPS6 | 6194 | CCR2+IL1B+ infiltrating macrophages inflammatory arthritis |
| | | synovium |
| RPS7 | 6201 | CCR2+IL1B+ infiltrating macrophages inflammatory arthritis |
| | | synovium |
| RPS9 | 6203 | CCR2+IL1B+ infiltrating macrophages inflammatory arthritis |
| | | synovium |
| RPSA | 3921 | CCR2+IL1B+ infiltrating macrophages inflammatory arthritis |
| | | synovium |
| RUNX3 | 864 | CCR2+IL1B+ infiltrating macrophages inflammatory arthritis |
| | | synovium |
| S100A11 | 6282 | CCR2+IL1B+ infiltrating macrophages inflammatory arthritis |
| | | synovium |
| SAMHD1 | 25939 | CCR2+IL1B+ infiltrating macrophages inflammatory arthritis |
| | | synovium |
| SBNO2 | 22904 | CCR2+IL1B+ infiltrating macrophages inflammatory arthritis |
| | | synovium |
| SCAND1 | 51282 | CCR2+IL1B+ infiltrating macrophages inflammatory arthritis |
| | | synovium |
| SDE2 | 163859 | CCR2+IL1B+ infiltrating macrophages inflammatory arthritis |
| | | synovium |
| SEC61B | 10952 | CCR2+IL1B+ infiltrating macrophages inflammatory arthritis |
| | | synovium |
| SEC61G | 23480 | CCR2+IL1B+ infiltrating macrophages inflammatory arthritis |
| | | synovium |
| SELPLG | 6404 | CCR2+IL1B+ infiltrating macrophages inflammatory arthritis |
| | | synovium |
| SERP1 | 27230 | CCR2+IL1B+ infiltrating macrophages inflammatory arthritis |
| | | synovium |
| SF3B1 | 23451 | CCR2+IL1B+ infiltrating macrophages inflammatory arthritis |
| | | synovium |
| SF3B6 | 51639 | CCR2+IL1B+ infiltrating macrophages inflammatory arthritis |
| | | synovium |
| SHISA5 | 51246 | CCR2+IL1B+ infiltrating macrophages inflammatory arthritis |
| | | synovium |
| SIRPB1 | 10326 | CCR2+IL1B+ infiltrating macrophages inflammatory arthritis |
| | | synovium |
| SKAP2 | 8935 | CCR2+IL1B+ infiltrating macrophages inflammatory arthritis |
| | | synovium |
| SLC15A3 | 51296 | CCR2+IL1B+ infiltrating macrophages inflammatory arthritis |
| | | synovium |
| SLC16A3 | 9123 | CCR2+IL1B+ infiltrating macrophages inflammatory arthritis |
| | | synovium |
| SLC25A5 | 292 | CCR2+IL1B+ infiltrating macrophages inflammatory arthritis |
| | | synovium |
| SLC7A11 | 23657 | CCR2+IL1B+ infiltrating macrophages inflammatory arthritis |
| | | synovium |
| SLFN5 | 162394 | CCR2+IL1B+ infiltrating macrophages inflammatory arthritis |
| | | synovium |
| SMIM3 | 85027 | CCR2+IL1B+ infiltrating macrophages inflammatory arthritis |
| | | synovium |
| SMOX | 54498 | CCR2+IL1B+ infiltrating macrophages inflammatory arthritis |
| | | synovium |
| SMPDL3A | 10924 | CCR2+IL1B+ infiltrating macrophages inflammatory arthritis |
| | | synovium |
| SNX1 | 6642 | CCR2+IL1B+ infiltrating macrophages inflammatory arthritis |
| | | synovium |
| SOCS3 | 9021 | CCR2+IL1B+ infiltrating macrophages inflammatory arthritis |
| | | synovium |
| SOD2 | 6648 | CCR2+IL1B+ infiltrating macrophages inflammatory arthritis |
| | | synovium |
| SPCS2 | 9789 | CCR2+IL1B+ infiltrating macrophages inflammatory arthritis |
| | | synovium |
| SRGN | 5552 | CCR2+IL1B+ infiltrating macrophages inflammatory arthritis |
| | | synovium |
| SRSF3 | 6428 | CCR2+IL1B+ infiltrating macrophages inflammatory arthritis |
| | | synovium |
| SRSF5 | 6430 | CCR2+IL1B+ infiltrating macrophages inflammatory arthritis |
| | | synovium |
| SSR2 | 6746 | CCR2+IL1B+ infiltrating macrophages inflammatory arthritis |
| | | synovium |
| ST3GAL4 | 6484 | CCR2+IL1B+ infiltrating macrophages inflammatory arthritis |
| | | synovium |
| SUB1 | 10923 | CCR2+IL1B+ infiltrating macrophages inflammatory arthritis |
| | | synovium |
| SYS1 | 90196 | CCR2+IL1B+ infiltrating macrophages inflammatory arthritis |
| | | synovium |
| TALDO1 | 6888 | CCR2+IL1B+ infiltrating macrophages inflammatory arthritis |
| | | synovium |
| TARM1 | 441864 | CCR2+IL1B+ infiltrating macrophages inflammatory arthritis |
| | | synovium |
| TGFB1 | 7040 | CCR2+IL1B+ infiltrating macrophages inflammatory arthritis |
| | | synovium |
| TGFBI | 7045 | CCR2+IL1B+ infiltrating macrophages inflammatory arthritis |
| | | synovium |
| TGIF1 | 7050 | CCR2+IL1B+ infiltrating macrophages inflammatory arthritis |
| | | synovium |
| TGM2 | 7052 | CCR2+IL1B+ infiltrating macrophages inflammatory arthritis |
| | | synovium |
| THBS1 | 7057 | CCR2+IL1B+ infiltrating macrophages inflammatory arthritis |
| | | synovium |
| TKT | 7086 | CCR2+IL1B+ infiltrating macrophages inflammatory arthritis |
| | | synovium |
| TLR2 | 7097 | CCR2+IL1B+ infiltrating macrophages inflammatory arthritis |
| | | synovium |
| TM6SF1 | 53346 | CCR2+IL1B+ infiltrating macrophages inflammatory arthritis |
| | | synovium |
| TMA7 | 51372 | CCR2+IL1B+ infiltrating macrophages inflammatory arthritis |
| | | synovium |
| TMBIM6 | 7009 | CCR2+IL1B+ infiltrating macrophages inflammatory arthritis |
| | | synovium |
| TMED2 | 10959 | CCR2+IL1B+ infiltrating macrophages inflammatory arthritis |
| | | synovium |
| TMED5 | 50999 | CCR2+IL1B+ infiltrating macrophages inflammatory arthritis |
| | | synovium |
| TMEM14C | 51522 | CCR2+IL1B+ infiltrating macrophages inflammatory arthritis |
| | | synovium |
| TMEM167A | 153339 | CCR2+IL1B+ infiltrating macrophages inflammatory arthritis |
| | | synovium |
| TMEM258 | 746 | CCR2+IL1B+ infiltrating macrophages inflammatory arthritis |
| | | synovium |
| TMSB10 | 9168 | CCR2+IL1B+ infiltrating macrophages inflammatory arthritis |
| | | synovium |
| TNFRSF1A | 7132 | CCR2+IL1B+ infiltrating macrophages inflammatory arthritis |
| | | synovium |
| TOR1A | 1861 | CCR2+IL1B+ infiltrating macrophages inflammatory arthritis |
| | | synovium |
| TOR1AIP1 | 26092 | CCR2+IL1B+ infiltrating macrophages inflammatory arthritis |
| | | synovium |
| TPD52 | 7163 | CCR2+IL1B+ infiltrating macrophages inflammatory arthritis |
| | | synovium |
| TPM4 | 7171 | CCR2+IL1B+ infiltrating macrophages inflammatory arthritis |
| | | synovium |
| TPST2 | 8459 | CCR2+IL1B+ infiltrating macrophages inflammatory arthritis |
| | | synovium |
| TRAF1 | 7185 | CCR2+IL1B+ infiltrating macrophages inflammatory arthritis |
| | | synovium |
| TRAM1 | 23471 | CCR2+IL1B+ infiltrating macrophages inflammatory arthritis |
| | | synovium |
| TREM1 | 54210 | CCR2+IL1B+ infiltrating macrophages inflammatory arthritis |
| | | synovium |
| TRIB1 | 10221 | CCR2+IL1B+ infiltrating macrophages inflammatory arthritis |
| | | synovium |
| TRPS1 | 7227 | CCR2+IL1B+ infiltrating macrophages inflammatory arthritis |
| | | synovium |
| TSPO | 706 | CCR2+IL1B+ infiltrating macrophages inflammatory arthritis |
| | | synovium |
| TWF2 | 11344 | CCR2+IL1B+ infiltrating macrophages inflammatory arthritis |
| | | synovium |
| TYROBP | 7305 | CCR2+IL1B+ infiltrating macrophages inflammatory arthritis |
| | | synovium |
| UBA52 | 7311 | CCR2+IL1B+ infiltrating macrophages inflammatory arthritis |
| | | synovium |
| UBC | 7316 | CCR2+IL1B+ infiltrating macrophages inflammatory arthritis |
| | | synovium |
| UBE2D3 | 7323 | CCR2+IL1B+ infiltrating macrophages inflammatory arthritis |
| | | synovium |
| UBE2L3 | 7332 | CCR2+IL1B+ infiltrating macrophages inflammatory arthritis |
| | | synovium |
| UBE2N | 7334 | CCR2+IL1B+ infiltrating macrophages inflammatory arthritis |
| | | synovium |
| UCK2 | 7371 | CCR2+IL1B+ infiltrating macrophages inflammatory arthritis |
| | | synovium |
| UCP2 | 7351 | CCR2+IL1B+ infiltrating macrophages inflammatory arthritis |
| | | synovium |
| UQCRC1 | 7384 | CCR2+IL1B+ infiltrating macrophages inflammatory arthritis |
| | | synovium |
| VAMP4 | 8674 | CCR2+IL1B+ infiltrating macrophages inflammatory arthritis |
| | | synovium |
| VASP | 7408 | CCR2+IL1B+ infiltrating macrophages inflammatory arthritis |
| | | synovium |
| VCAN | 1462 | CCR2+IL1B+ infiltrating macrophages inflammatory arthritis |
| | | synovium |
| VMP1 | 81671 | CCR2+IL1B+ infiltrating macrophages inflammatory arthritis |
| | | synovium |
| XBP1 | 7494 | CCR2+IL1B+ infiltrating macrophages inflammatory arthritis |
| | | synovium |
| ZBP1 | 81030 | CCR2+IL1B+ infiltrating macrophages inflammatory arthritis |
| | | synovium |
| ZEB2 | 9839 | CCR2+IL1B+ infiltrating macrophages inflammatory arthritis |
| | | synovium |
| ZFP36 | 7538 | CCR2+IL1B+ infiltrating macrophages inflammatory arthritis |
| | | synovium |
| ZNF705A | 440077 | CCR2+IL1B+ infiltrating macrophages inflammatory arthritis |
| | | synovium |
| ZYX | 7791 | CCR2+IL1B+ infiltrating macrophages inflammatory arthritis |
| | | synovium |
| ABCA1 | 19 | CX3CR1+ lining macrophages inflammatory arthritis synovium |
| ABHD12 | 26090 | CX3CR1+ lining macrophages inflammatory arthritis synovium |
| ADAM15 | 8751 | CX3CR1+ lining macrophages inflammatory arthritis synovium |
| ADGRE5 | 976 | CX3CR1+ lining macrophages inflammatory arthritis synovium |
| AHNAK2 | 113146 | CX3CR1+ lining macrophages inflammatory arthritis synovium |
| AKR1B1 | 231 | CX3CR1+ lining macrophages inflammatory arthritis synovium |
| ALDH2 | 217 | CX3CR1+ lining macrophages inflammatory arthritis synovium |
| ALOX5 | 240 | CX3CR1+ lining macrophages inflammatory arthritis synovium |
| ANG | 283 | CX3CR1+ lining macrophages inflammatory arthritis synovium |
| ANXA3 | 306 | CX3CR1+ lining macrophages inflammatory arthritis synovium |
| APOE | 348 | CX3CR1+ lining macrophages inflammatory arthritis synovium |
| APP | 351 | CX3CR1+ lining macrophages inflammatory arthritis synovium |
| ARL11 | 115761 | CX3CR1+ lining macrophages inflammatory arthritis synovium |
| ARSB | 411 | CX3CR1+ lining macrophages inflammatory arthritis synovium |
| ASAH1 | 427 | CX3CR1+ lining macrophages inflammatory arthritis synovium |
| ASS1 | 445 | CX3CR1+ lining macrophages inflammatory arthritis synovium |
| ATP6AP1 | 537 | CX3CR1+ lining macrophages inflammatory arthritis synovium |
| ATRAID | 51374 | CX3CR1+ lining macrophages inflammatory arthritis synovium |
| AXL | 558 | CX3CR1+ lining macrophages inflammatory arthritis synovium |
| B2M | 567 | CX3CR1+ lining macrophages inflammatory arthritis synovium |
| BCL2A1 | 597 | CX3CR1+ lining macrophages inflammatory arthritis synovium |
| BIN1 | 274 | CX3CR1+ lining macrophages inflammatory arthritis synovium |
| BLNK | 29760 | CX3CR1+ lining macrophages inflammatory arthritis synovium |
| BLVRB | 645 | CX3CR1+ lining macrophages inflammatory arthritis synovium |
| BMP2K | 55589 | CX3CR1+ lining macrophages inflammatory arthritis synovium |
| C1QA | 712 | CX3CR1+ lining macrophages inflammatory arthritis synovium |
| C1QB | 713 | CX3CR1+ lining macrophages inflammatory arthritis synovium |
| C1QC | 714 | CX3CR1+ lining macrophages inflammatory arthritis synovium |
| C3AR1 | 719 | CX3CR1+ lining macrophages inflammatory arthritis synovium |
| C3orf70 | 285382 | CX3CR1+ lining macrophages inflammatory arthritis synovium |
| C4B | 721 | CX3CR1+ lining macrophages inflammatory arthritis synovium |
| C9orf3 | 84909 | CX3CR1+ lining macrophages inflammatory arthritis synovium |
| CAMK1 | 8536 | CX3CR1+ lining macrophages inflammatory arthritis synovium |
| CC2D1B | 200014 | CX3CR1+ lining macrophages inflammatory arthritis synovium |
| CCL3L3 | 414062 | CX3CR1+ lining macrophages inflammatory arthritis synovium |
| CD109 | 135228 | CX3CR1+ lining macrophages inflammatory arthritis synovium |
| CD151 | 977 | CX3CR1+ lining macrophages inflammatory arthritis synovium |
| CD37 | 951 | CX3CR1+ lining macrophages inflammatory arthritis synovium |
| CD40 | 958 | CX3CR1+ lining macrophages inflammatory arthritis synovium |
| CD63 | 967 | CX3CR1+ lining macrophages inflammatory arthritis synovium |
| CD83 | 9308 | CX3CR1+ lining macrophages inflammatory arthritis synovium |
| CD84 | 8832 | CX3CR1+ lining macrophages inflammatory arthritis synovium |
| CD9 | 928 | CX3CR1+ lining macrophages inflammatory arthritis synovium |
| CDKN2C | 1031 | CX3CR1+ lining macrophages inflammatory arthritis synovium |
| CEBPA | 1050 | CX3CR1+ lining macrophages inflammatory arthritis synovium |
| CEBPG | 1054 | CX3CR1+ lining macrophages inflammatory arthritis synovium |
| CEBPZOS | 1.01E+08 | CX3CR1+ lining macrophages inflammatory arthritis synovium |
| CFH | 3075 | CX3CR1+ lining macrophages inflammatory arthritis synovium |
| CHD9 | 80205 | CX3CR1+ lining macrophages inflammatory arthritis synovium |
| CHST1 | 8534 | CX3CR1+ lining macrophages inflammatory arthritis synovium |
| CLEC12A | 160364 | CX3CR1+ lining macrophages inflammatory arthritis synovium |
| CLIC4 | 25932 | CX3CR1+ lining macrophages inflammatory arthritis synovium |
| COLEC12 | 81035 | CX3CR1+ lining macrophages inflammatory arthritis synovium |
| COMMD10 | 51397 | CX3CR1+ lining macrophages inflammatory arthritis synovium |
| COMT | 1312 | CX3CR1+ lining macrophages inflammatory arthritis synovium |
| COTL1 | 23406 | CX3CR1+ lining macrophages inflammatory arthritis synovium |
| CREG1 | 8804 | CX3CR1+ lining macrophages inflammatory arthritis synovium |
| CRLF2 | 64109 | CX3CR1+ lining macrophages inflammatory arthritis synovium |
| CSF1R | 1436 | CX3CR1+ lining macrophages inflammatory arthritis synovium |
| CSPG4 | 1464 | CX3CR1+ lining macrophages inflammatory arthritis synovium |
| CST3 | 1471 | CX3CR1+ lining macrophages inflammatory arthritis synovium |
| CTSA | 5476 | CX3CR1+ lining macrophages inflammatory arthritis synovium |
| CTSB | 1508 | CX3CR1+ lining macrophages inflammatory arthritis synovium |
| CTSD | 1509 | CX3CR1+ lining macrophages inflammatory arthritis synovium |
| CTSF | 8722 | CX3CR1+ lining macrophages inflammatory arthritis synovium |
| CTSS | 1520 | CX3CR1+ lining macrophages inflammatory arthritis synovium |
| CX3CR1 | 1524 | CX3CR1+ lining macrophages inflammatory arthritis synovium |
| CYB5A | 1528 | CX3CR1+ lining macrophages inflammatory arthritis synovium |
| CYFIP1 | 23191 | CX3CR1+ lining macrophages inflammatory arthritis synovium |
| DDX3X | 1654 | CX3CR1+ lining macrophages inflammatory arthritis synovium |
| DHRS3 | 9249 | CX3CR1+ lining macrophages inflammatory arthritis synovium |
| DHRS7 | 51635 | CX3CR1+ lining macrophages inflammatory arthritis synovium |
| DNASE2 | 1777 | CX3CR1+ lining macrophages inflammatory arthritis synovium |
| DOK3 | 79930 | CX3CR1+ lining macrophages inflammatory arthritis synovium |
| DPP7 | 29952 | CX3CR1+ lining macrophages inflammatory arthritis synovium |
| DSTN | 11034 | CX3CR1+ lining macrophages inflammatory arthritis synovium |
| DTNBP1 | 84062 | CX3CR1+ lining macrophages inflammatory arthritis synovium |
| DUSP3 | 1845 | CX3CR1+ lining macrophages inflammatory arthritis synovium |
| ECM1 | 1893 | CX3CR1+ lining macrophages inflammatory arthritis synovium |
| ECSCR | 641700 | CX3CR1+ lining macrophages inflammatory arthritis synovium |
| EGR1 | 1958 | CX3CR1+ lining macrophages inflammatory arthritis synovium |
| ELMO1 | 9844 | CX3CR1+ lining macrophages inflammatory arthritis synovium |
| EMP3 | 2014 | CX3CR1+ lining macrophages inflammatory arthritis synovium |
| ENPP1 | 5167 | CX3CR1+ lining macrophages inflammatory arthritis synovium |
| ENTPD1 | 953 | CX3CR1+ lining macrophages inflammatory arthritis synovium |
| ERGIC3 | 51614 | CX3CR1+ lining macrophages inflammatory arthritis synovium |
| F11R | 50848 | CX3CR1+ lining macrophages inflammatory arthritis synovium |
| FABP3 | 2170 | CX3CR1+ lining macrophages inflammatory arthritis synovium |
| FAM213B | 127281 | CX3CR1+ lining macrophages inflammatory arthritis synovium |
| FAM3C | 10447 | CX3CR1+ lining macrophages inflammatory arthritis synovium |
| FBXW4 | 6468 | CX3CR1+ lining macrophages inflammatory arthritis synovium |
| FCGRT | 2217 | CX3CR1+ lining macrophages inflammatory arthritis synovium |
| FCHSD2 | 9873 | CX3CR1+ lining macrophages inflammatory arthritis synovium |
| FERMT2 | 10979 | CX3CR1+ lining macrophages inflammatory arthritis synovium |
| FEZ2 | 9637 | CX3CR1+ lining macrophages inflammatory arthritis synovium |
| FN1 | 2335 | CX3CR1+ lining macrophages inflammatory arthritis synovium |
| FOLR2 | 2350 | CX3CR1+ lining macrophages inflammatory arthritis synovium |
| FOSB | 2354 | CX3CR1+ lining macrophages inflammatory arthritis synovium |
| FTH1 | 2495 | CX3CR1+ lining macrophages inflammatory arthritis synovium |
| FTL | 2512 | CX3CR1+ lining macrophages inflammatory arthritis synovium |
| FUCA1 | 2517 | CX3CR1+ lining macrophages inflammatory arthritis synovium |
| GAS6 | 2621 | CX3CR1+ lining macrophages inflammatory arthritis synovium |
| GLMP | 112770 | CX3CR1+ lining macrophages inflammatory arthritis synovium |
| GLTP | 51228 | CX3CR1+ lining macrophages inflammatory arthritis synovium |
| GLUL | 2752 | CX3CR1+ lining macrophages inflammatory arthritis synovium |
| GNA15 | 2769 | CX3CR1+ lining macrophages inflammatory arthritis synovium |
| GNAS | 2778 | CX3CR1+ lining macrophages inflammatory arthritis synovium |
| GNG5 | 2787 | CX3CR1+ lining macrophages inflammatory arthritis synovium |
| GNS | 2799 | CX3CR1+ lining macrophages inflammatory arthritis synovium |
| GPR34 | 2857 | CX3CR1+ lining macrophages inflammatory arthritis synovium |
| GPX1 | 2876 | CX3CR1+ lining macrophages inflammatory arthritis synovium |
| GPX4 | 2879 | CX3CR1+ lining macrophages inflammatory arthritis synovium |
| GRN | 2896 | CX3CR1+ lining macrophages inflammatory arthritis synovium |
| GUSB | 2990 | CX3CR1+ lining macrophages inflammatory arthritis synovium |
| HEXA | 3073 | CX3CR1+ lining macrophages inflammatory arthritis synovium |
| HEXB | 3074 | CX3CR1+ lining macrophages inflammatory arthritis synovium |
| HLA-A | 3105 | CX3CR1+ lining macrophages inflammatory arthritis synovium |
| HPGD | 3248 | CX3CR1+ lining macrophages inflammatory arthritis synovium |
| HPGDS | 27306 | CX3CR1+ lining macrophages inflammatory arthritis synovium |
| HSBP1 | 3281 | CX3CR1+ lining macrophages inflammatory arthritis synovium |
| IER2 | 9592 | CX3CR1+ lining macrophages inflammatory arthritis synovium |
| IGF1 | 3479 | CX3CR1+ lining macrophages inflammatory arthritis synovium |
| IL11RA | 3590 | CX3CR1+ lining macrophages inflammatory arthritis synovium |
| ITGAV | 3685 | CX3CR1+ lining macrophages inflammatory arthritis synovium |
| ITGB1 | 3688 | CX3CR1+ lining macrophages inflammatory arthritis synovium |
| ITGB2 | 3689 | CX3CR1+ lining macrophages inflammatory arthritis synovium |
| ITGB5 | 3693 | CX3CR1+ lining macrophages inflammatory arthritis synovium |
| ITM2B | 9445 | CX3CR1+ lining macrophages inflammatory arthritis synovium |
| ITM2C | 81618 | CX3CR1+ lining macrophages inflammatory arthritis synovium |
| KCNK13 | 56659 | CX3CR1+ lining macrophages inflammatory arthritis synovium |
| KLC1 | 3831 | CX3CR1+ lining macrophages inflammatory arthritis synovium |
| KLF2 | 10365 | CX3CR1+ lining macrophages inflammatory arthritis synovium |
| KLF4 | 9314 | CX3CR1+ lining macrophages inflammatory arthritis synovium |
| KLHL6 | 89857 | CX3CR1+ lining macrophages inflammatory arthritis synovium |
| LAMP1 | 3916 | CX3CR1+ lining macrophages inflammatory arthritis synovium |
| LAMP2 | 3920 | CX3CR1+ lining macrophages inflammatory arthritis synovium |
| LAMTOR1 | 55004 | CX3CR1+ lining macrophages inflammatory arthritis synovium |
| LAPTM4A | 9741 | CX3CR1+ lining macrophages inflammatory arthritis synovium |
| LGALS9B | 284194 | CX3CR1+ lining macrophages inflammatory arthritis synovium |
| LGMN | 5641 | CX3CR1+ lining macrophages inflammatory arthritis synovium |
| LIPA | 3988 | CX3CR1+ lining macrophages inflammatory arthritis synovium |
| LMO2 | 4005 | CX3CR1+ lining macrophages inflammatory arthritis synovium |
| LPCAT2 | 54947 | CX3CR1+ lining macrophages inflammatory arthritis synovium |
| LRP1 | 4035 | CX3CR1+ lining macrophages inflammatory arthritis synovium |
| LTC4S | 4056 | CX3CR1+ lining macrophages inflammatory arthritis synovium |
| LUZP1 | 7798 | CX3CR1+ lining macrophages inflammatory arthritis synovium |
| LY86 | 9450 | CX3CR1+ lining macrophages inflammatory arthritis synovium |
| LYL1 | 4066 | CX3CR1+ lining macrophages inflammatory arthritis synovium |
| LYVE1 | 10894 | CX3CR1+ lining macrophages inflammatory arthritis synovium |
| LYZ | 4069 | CX3CR1+ lining macrophages inflammatory arthritis synovium |
| MACF1 | 23499 | CX3CR1+ lining macrophages inflammatory arthritis synovium |
| MAF | 4094 | CX3CR1+ lining macrophages inflammatory arthritis synovium |
| MAN2B1 | 4125 | CX3CR1+ lining macrophages inflammatory arthritis synovium |
| MAP1LC3A | 84557 | CX3CR1+ lining macrophages inflammatory arthritis synovium |
| MAPK3 | 5595 | CX3CR1+ lining macrophages inflammatory arthritis synovium |
| MARCKS | 4082 | CX3CR1+ lining macrophages inflammatory arthritis synovium |
| MDH1 | 4190 | CX3CR1+ lining macrophages inflammatory arthritis synovium |
| MEF2A | 4205 | CX3CR1+ lining macrophages inflammatory arthritis synovium |
| MEF2C | 4208 | CX3CR1+ lining macrophages inflammatory arthritis synovium |
| MFGE8 | 4240 | CX3CR1+ lining macrophages inflammatory arthritis synovium |
| MGST1 | 4257 | CX3CR1+ lining macrophages inflammatory arthritis synovium |
| MGST3 | 4259 | CX3CR1+ lining macrophages inflammatory arthritis synovium |
| MOB2 | 81532 | CX3CR1+ lining macrophages inflammatory arthritis synovium |
| MPP1 | 4354 | CX3CR1+ lining macrophages inflammatory arthritis synovium |
| MS4A7 | 58475 | CX3CR1+ lining macrophages inflammatory arthritis synovium |
| MT-ND2 | 4536 | CX3CR1+ lining macrophages inflammatory arthritis synovium |
| MTDH | 92140 | CX3CR1+ lining macrophages inflammatory arthritis synovium |
| MTUS1 | 57509 | CX3CR1+ lining macrophages inflammatory arthritis synovium |
| NAGK | 55577 | CX3CR1+ lining macrophages inflammatory arthritis synovium |
| NCF1 | 653361 | CX3CR1+ lining macrophages inflammatory arthritis synovium |
| NECAP2 | 55707 | CX3CR1+ lining macrophages inflammatory arthritis synovium |
| NFIA | 4774 | CX3CR1+ lining macrophages inflammatory arthritis synovium |
| NFIC | 4782 | CX3CR1+ lining macrophages inflammatory arthritis synovium |
| NINJ1 | 4814 | CX3CR1+ lining macrophages inflammatory arthritis synovium |
| NPC2 | 10577 | CX3CR1+ lining macrophages inflammatory arthritis synovium |
| NPL | 80896 | CX3CR1+ lining macrophages inflammatory arthritis synovium |
| NPTN | 27020 | CX3CR1+ lining macrophages inflammatory arthritis synovium |
| NRP1 | 8829 | CX3CR1+ lining macrophages inflammatory arthritis synovium |
| NTPCR | 84284 | CX3CR1+ lining macrophages inflammatory arthritis synovium |
| NUCB1 | 4924 | CX3CR1+ lining macrophages inflammatory arthritis synovium |
| NUPR1 | 26471 | CX3CR1+ lining macrophages inflammatory arthritis synovium |
| OLFML3 | 56944 | CX3CR1+ lining macrophages inflammatory arthritis synovium |
| OPHN1 | 4983 | CX3CR1+ lining macrophages inflammatory arthritis synovium |
| P2RX4 | 5025 | CX3CR1+ lining macrophages inflammatory arthritis synovium |
| P2RY12 | 64805 | CX3CR1+ lining macrophages inflammatory arthritis synovium |
| P2RY6 | 5031 | CX3CR1+ lining macrophages inflammatory arthritis synovium |
| PALD1 | 27143 | CX3CR1+ lining macrophages inflammatory arthritis synovium |
| PBXIP1 | 57326 | CX3CR1+ lining macrophages inflammatory arthritis synovium |
| PDLIM2 | 64236 | CX3CR1+ lining macrophages inflammatory arthritis synovium |
| PDLIM7 | 9260 | CX3CR1+ lining macrophages inflammatory arthritis synovium |
| PEPD | 5184 | CX3CR1+ lining macrophages inflammatory arthritis synovium |
| PKIB | 5570 | CX3CR1+ lining macrophages inflammatory arthritis synovium |
| PLA2G15 | 23659 | CX3CR1+ lining macrophages inflammatory arthritis synovium |
| PLA2G16 | 11145 | CX3CR1+ lining macrophages inflammatory arthritis synovium |
| PLBD2 | 196463 | CX3CR1+ lining macrophages inflammatory arthritis synovium |
| PLOD1 | 5351 | CX3CR1+ lining macrophages inflammatory arthritis synovium |
| PLTP | 5360 | CX3CR1+ lining macrophages inflammatory arthritis synovium |
| PLXNB2 | 23654 | CX3CR1+ lining macrophages inflammatory arthritis synovium |
| PMEPA1 | 56937 | CX3CR1+ lining macrophages inflammatory arthritis synovium |
| PMP22 | 5376 | CX3CR1+ lining macrophages inflammatory arthritis synovium |
| PNKD | 25953 | CX3CR1+ lining macrophages inflammatory arthritis synovium |
| POLR2E | 5434 | CX3CR1+ lining macrophages inflammatory arthritis synovium |
| PON2 | 5445 | CX3CR1+ lining macrophages inflammatory arthritis synovium |
| PON3 | 5446 | CX3CR1+ lining macrophages inflammatory arthritis synovium |
| PRUNE2 | 158471 | CX3CR1+ lining macrophages inflammatory arthritis synovium |
| PTPRA | 5786 | CX3CR1+ lining macrophages inflammatory arthritis synovium |
| QKI | 9444 | CX3CR1+ lining macrophages inflammatory arthritis synovium |
| QPCT | 25797 | CX3CR1+ lining macrophages inflammatory arthritis synovium |
| RAB11A | 8766 | CX3CR1+ lining macrophages inflammatory arthritis synovium |
| RAB11FIP5 | 26056 | CX3CR1+ lining macrophages inflammatory arthritis synovium |
| RAB31 | 11031 | CX3CR1+ lining macrophages inflammatory arthritis synovium |
| RAB3IL1 | 5866 | CX3CR1+ lining macrophages inflammatory arthritis synovium |
| RABAC1 | 10567 | CX3CR1+ lining macrophages inflammatory arthritis synovium |
| RASGEF1B | 153020 | CX3CR1+ lining macrophages inflammatory arthritis synovium |
| RASGRP3 | 25780 | CX3CR1+ lining macrophages inflammatory arthritis synovium |
| RENBP | 5973 | CX3CR1+ lining macrophages inflammatory arthritis synovium |
| RFTN1 | 23180 | CX3CR1+ lining macrophages inflammatory arthritis synovium |
| RGS10 | 6001 | CX3CR1+ lining macrophages inflammatory arthritis synovium |
| RHOA | 387 | CX3CR1+ lining macrophages inflammatory arthritis synovium |
| RHOB | 388 | CX3CR1+ lining macrophages inflammatory arthritis synovium |
| RHOC | 389 | CX3CR1+ lining macrophages inflammatory arthritis synovium |
| RIN2 | 54453 | CX3CR1+ lining macrophages inflammatory arthritis synovium |
| RNASE4 | 6038 | CX3CR1+ lining macrophages inflammatory arthritis synovium |
| RNASET2 | 8635 | CX3CR1+ lining macrophages inflammatory arthritis synovium |
| RNF13 | 11342 | CX3CR1+ lining macrophages inflammatory arthritis synovium |
| RTCB | 51493 | CX3CR1+ lining macrophages inflammatory arthritis synovium |
| S100A1 | 6271 | CX3CR1+ lining macrophages inflammatory arthritis synovium |
| S100A13 | 6284 | CX3CR1+ lining macrophages inflammatory arthritis synovium |
| S100B | 6285 | CX3CR1+ lining macrophages inflammatory arthritis synovium |
| S1PR1 | 1901 | CX3CR1+ lining macrophages inflammatory arthritis synovium |
| SAMD9L | 219285 | CX3CR1+ lining macrophages inflammatory arthritis synovium |
| SARAF | 51669 | CX3CR1+ lining macrophages inflammatory arthritis synovium |
| SASH1 | 23328 | CX3CR1+ lining macrophages inflammatory arthritis synovium |
| SAT1 | 6303 | CX3CR1+ lining macrophages inflammatory arthritis synovium |
| SCAMP2 | 10066 | CX3CR1+ lining macrophages inflammatory arthritis synovium |
| SCOC | 60592 | CX3CR1+ lining macrophages inflammatory arthritis synovium |
| SEC14L1 | 6397 | CX3CR1+ lining macrophages inflammatory arthritis synovium |
| SELENBP1 | 8991 | CX3CR1+ lining macrophages inflammatory arthritis synovium |
| SELENOM | 140606 | CX3CR1+ lining macrophages inflammatory arthritis synovium |
| SELENOP | 6414 | CX3CR1+ lining macrophages inflammatory arthritis synovium |
| SELENOW | 6415 | CX3CR1+ lining macrophages inflammatory arthritis synovium |
| SERINC3 | 10955 | CX3CR1+ lining macrophages inflammatory arthritis synovium |
| SERPINB6 | 5269 | CX3CR1+ lining macrophages inflammatory arthritis synovium |
| SERPINE1 | 5054 | CX3CR1+ lining macrophages inflammatory arthritis synovium |
| SESN1 | 27244 | CX3CR1+ lining macrophages inflammatory arthritis synovium |
| SFXN3 | 81855 | CX3CR1+ lining macrophages inflammatory arthritis synovium |
| SGK1 | 6446 | CX3CR1+ lining macrophages inflammatory arthritis synovium |
| SH3BP5 | 9467 | CX3CR1+ lining macrophages inflammatory arthritis synovium |
| SH3GLB1 | 51100 | CX3CR1+ lining macrophages inflammatory arthritis synovium |
| SIGMAR1 | 10280 | CX3CR1+ lining macrophages inflammatory arthritis synovium |
| SIRT2 | 22933 | CX3CR1+ lining macrophages inflammatory arthritis synovium |
| SKI | 6497 | CX3CR1+ lining macrophages inflammatory arthritis synovium |
| SKIL | 6498 | CX3CR1+ lining macrophages inflammatory arthritis synovium |
| SLC27A1 | 376497 | CX3CR1+ lining macrophages inflammatory arthritis synovium |
| SLC29A1 | 2030 | CX3CR1+ lining macrophages inflammatory arthritis synovium |
| SLC2A6 | 11182 | CX3CR1+ lining macrophages inflammatory arthritis synovium |
| SLC6A6 | 6533 | CX3CR1+ lining macrophages inflammatory arthritis synovium |
| SLC9A3R2 | 9351 | CX3CR1+ lining macrophages inflammatory arthritis synovium |
| SLCO2B1 | 11309 | CX3CR1+ lining macrophages inflammatory arthritis synovium |
| SMAGP | 57228 | CX3CR1+ lining macrophages inflammatory arthritis synovium |
| SMIM14 | 201895 | CX3CR1+ lining macrophages inflammatory arthritis synovium |
| SNX3 | 8724 | CX3CR1+ lining macrophages inflammatory arthritis synovium |
| SPARC | 6678 | CX3CR1+ lining macrophages inflammatory arthritis synovium |
| SPTBN1 | 6711 | CX3CR1+ lining macrophages inflammatory arthritis synovium |
| SPTSSA | 171546 | CX3CR1+ lining macrophages inflammatory arthritis synovium |
| SRGAP2 | 23380 | CX3CR1+ lining macrophages inflammatory arthritis synovium |
| SSBP4 | 170463 | CX3CR1+ lining macrophages inflammatory arthritis synovium |
| ST3GAL5 | 8869 | CX3CR1+ lining macrophages inflammatory arthritis synovium |
| STAB1 | 23166 | CX3CR1+ lining macrophages inflammatory arthritis synovium |
| STOM | 2040 | CX3CR1+ lining macrophages inflammatory arthritis synovium |
| STX4 | 6810 | CX3CR1+ lining macrophages inflammatory arthritis synovium |
| SULF2 | 55959 | CX3CR1+ lining macrophages inflammatory arthritis synovium |
| SYNGR1 | 9145 | CX3CR1+ lining macrophages inflammatory arthritis synovium |
| TBXAS1 | 6916 | CX3CR1+ lining macrophages inflammatory arthritis synovium |
| TCIM | 56892 | CX3CR1+ lining macrophages inflammatory arthritis synovium |
| TCN2 | 6948 | CX3CR1+ lining macrophages inflammatory arthritis synovium |
| TECR | 9524 | CX3CR1+ lining macrophages inflammatory arthritis synovium |
| TGFBR1 | 7046 | CX3CR1+ lining macrophages inflammatory arthritis synovium |
| TGFBR2 | 7048 | CX3CR1+ lining macrophages inflammatory arthritis synovium |
| TIMD4 | 91937 | CX3CR1+ lining macrophages inflammatory arthritis synovium |
| TIMP2 | 7077 | CX3CR1+ lining macrophages inflammatory arthritis synovium |
| TMBIM1 | 64114 | CX3CR1+ lining macrophages inflammatory arthritis synovium |
| TMEM141 | 85014 | CX3CR1+ lining macrophages inflammatory arthritis synovium |
| TMEM176B | 28959 | CX3CR1+ lining macrophages inflammatory arthritis synovium |
| TMEM37 | 140738 | CX3CR1+ lining macrophages inflammatory arthritis synovium |
| TMEM50A | 23585 | CX3CR1+ lining macrophages inflammatory arthritis synovium |
| TMEM59 | 9528 | CX3CR1+ lining macrophages inflammatory arthritis synovium |
| TMEM86A | 144110 | CX3CR1+ lining macrophages inflammatory arthritis synovium |
| TMEM9B | 56674 | CX3CR1+ lining macrophages inflammatory arthritis synovium |
| TMSB4X | 7114 | CX3CR1+ lining macrophages inflammatory arthritis synovium |
| TNFRSF21 | 27242 | CX3CR1+ lining macrophages inflammatory arthritis synovium |
| TNFSF12 | 8742 | CX3CR1+ lining macrophages inflammatory arthritis synovium |
| TNS1 | 7145 | CX3CR1+ lining macrophages inflammatory arthritis synovium |
| TPP1 | 1200 | CX3CR1+ lining macrophages inflammatory arthritis synovium |
| TREM2 | 54209 | CX3CR1+ lining macrophages inflammatory arthritis synovium |
| TSC22D3 | 1831 | CX3CR1+ lining macrophages inflammatory arthritis synovium |
| TSPAN3 | 10099 | CX3CR1+ lining macrophages inflammatory arthritis synovium |
| TSPAN4 | 7106 | CX3CR1+ lining macrophages inflammatory arthritis synovium |
| TUBA1A | 7846 | CX3CR1+ lining macrophages inflammatory arthritis synovium |
| UBXN1 | 51035 | CX3CR1+ lining macrophages inflammatory arthritis synovium |
| UCHL3 | 7347 | CX3CR1+ lining macrophages inflammatory arthritis synovium |
| VAT1 | 10493 | CX3CR1+ lining macrophages inflammatory arthritis synovium |
| VSIG4 | 11326 | CX3CR1+ lining macrophages inflammatory arthritis synovium |
| YPEL3 | 83719 | CX3CR1+ lining macrophages inflammatory arthritis synovium |
| ZBTB20 | 26137 | CX3CR1+ lining macrophages inflammatory arthritis synovium |
| ZFHX3 | 463 | CX3CR1+ lining macrophages inflammatory arthritis synovium |
| ZMIZ1 | 57178 | CX3CR1+ lining macrophages inflammatory arthritis synovium |
| ABCA9 | 10350 | RELM-α+ interstitial macrophages inflammatory arthritis synovium |
| ABHD12 | 26090 | RELM-α+ interstitial macrophages inflammatory arthritis synovium |
| ACAT1 | 38 | RELM-α+ interstitial macrophages inflammatory arthritis synovium |
| ACOT13 | 55856 | RELM-α+ interstitial macrophages inflammatory arthritis synovium |
| AHNAK | 79026 | RELM-α+ interstitial macrophages inflammatory arthritis synovium |
| AHNAK2 | 113146 | RELM-α+ interstitial macrophages inflammatory arthritis synovium |
| AKR1B1 | 231 | RELM-α+ interstitial macrophages inflammatory arthritis synovium |
| ALDH2 | 217 | RELM-α+ interstitial macrophages inflammatory arthritis synovium |
| ALOX5 | 240 | RELM-α+ interstitial macrophages inflammatory arthritis synovium |
| ANG | 283 | RELM-α+ interstitial macrophages inflammatory arthritis synovium |
| ANP32A | 8125 | RELM-α+ interstitial macrophages inflammatory arthritis synovium |
| ANXA11 | 311 | RELM-α+ interstitial macrophages inflammatory arthritis synovium |
| ANXA6 | 309 | RELM-α+ interstitial macrophages inflammatory arthritis synovium |
| AP1B1 | 162 | RELM-α+ interstitial macrophages inflammatory arthritis synovium |
| AP2A2 | 161 | RELM-α+ interstitial macrophages inflammatory arthritis synovium |
| AP2M1 | 1173 | RELM-α+ interstitial macrophages inflammatory arthritis synovium |
| APLP2 | 334 | RELM-α+ interstitial macrophages inflammatory arthritis synovium |
| AQP1 | 358 | RELM-α+ interstitial macrophages inflammatory arthritis synovium |
| ATRAID | 51374 | RELM-α+ interstitial macrophages inflammatory arthritis synovium |
| BIN1 | 274 | RELM-α+ interstitial macrophages inflammatory arthritis synovium |
| BLVRB | 645 | RELM-α+ interstitial macrophages inflammatory arthritis synovium |
| BORCS6 | 54785 | RELM-α+ interstitial macrophages inflammatory arthritis synovium |
| C16orf54 | 283897 | RELM-α+ interstitial macrophages inflammatory arthritis synovium |
| C1QA | 712 | RELM-α+ interstitial macrophages inflammatory arthritis synovium |
| C1QB | 713 | RELM-α+ interstitial macrophages inflammatory arthritis synovium |
| C1QC | 714 | RELM-α+ interstitial macrophages inflammatory arthritis synovium |
| C4B | 721 | RELM-α+ interstitial macrophages inflammatory arthritis synovium |
| CALM2 | 805 | RELM-α+ interstitial macrophages inflammatory arthritis synovium |
| CAVIN1 | 284119 | RELM-α+ interstitial macrophages inflammatory arthritis synovium |
| CCL24 | 6369 | RELM-α+ interstitial macrophages inflammatory arthritis synovium |
| CD163 | 9332 | RELM-α+ interstitial macrophages inflammatory arthritis synovium |
| CD302 | 9936 | RELM-α+ interstitial macrophages inflammatory arthritis synovium |
| CD36 | 948 | RELM-α+ interstitial macrophages inflammatory arthritis synovium |
| CD37 | 951 | RELM-α+ interstitial macrophages inflammatory arthritis synovium |
| CD48 | 962 | RELM-α+ interstitial macrophages inflammatory arthritis synovium |
| CD63 | 967 | RELM-α+ interstitial macrophages inflammatory arthritis synovium |
| CD74 | 972 | RELM-α+ interstitial macrophages inflammatory arthritis synovium |
| CD81 | 975 | RELM-α+ interstitial macrophages inflammatory arthritis synovium |
| CDKN2C | 1031 | RELM-α+ interstitial macrophages inflammatory arthritis synovium |
| CFH | 3075 | RELM-α+ interstitial macrophages inflammatory arthritis synovium |
| CLEC10A | 10462 | RELM-α+ interstitial macrophages inflammatory arthritis synovium |
| CLN8 | 2055 | RELM-α+ interstitial macrophages inflammatory arthritis synovium |
| CLTA | 1211 | RELM-α+ interstitial macrophages inflammatory arthritis synovium |
| CLTB | 1212 | RELM-α+ interstitial macrophages inflammatory arthritis synovium |
| CLTC | 1213 | RELM-α+ interstitial macrophages inflammatory arthritis synovium |
| CNPY2 | 10330 | RELM-α+ interstitial macrophages inflammatory arthritis synovium |
| COLEC12 | 81035 | RELM-α+ interstitial macrophages inflammatory arthritis synovium |
| COMT | 1312 | RELM-α+ interstitial macrophages inflammatory arthritis synovium |
| CRACR2B | 283229 | RELM-α+ interstitial macrophages inflammatory arthritis synovium |
| CSF1R | 1436 | RELM-α+ interstitial macrophages inflammatory arthritis synovium |
| CST3 | 1471 | RELM-α+ interstitial macrophages inflammatory arthritis synovium |
| CTSB | 1508 | RELM-α+ interstitial macrophages inflammatory arthritis synovium |
| CTSC | 1075 | RELM-α+ interstitial macrophages inflammatory arthritis synovium |
| CYB5A | 1528 | RELM-α+ interstitial macrophages inflammatory arthritis synovium |
| CYB5R3 | 1727 | RELM-α+ interstitial macrophages inflammatory arthritis synovium |
| DAB2 | 1601 | RELM-α+ interstitial macrophages inflammatory arthritis synovium |
| DGUOK | 1716 | RELM-α+ interstitial macrophages inflammatory arthritis synovium |
| DHRS3 | 9249 | RELM-α+ interstitial macrophages inflammatory arthritis synovium |
| DNASE1L1 | 1774 | RELM-α+ interstitial macrophages inflammatory arthritis synovium |
| DOK2 | 9046 | RELM-α+ interstitial macrophages inflammatory arthritis synovium |
| DSE | 29940 | RELM-α+ interstitial macrophages inflammatory arthritis synovium |
| DSTN | 11034 | RELM-α+ interstitial macrophages inflammatory arthritis synovium |
| DUSP22 | 56940 | RELM-α+ interstitial macrophages inflammatory arthritis synovium |
| DUSP6 | 1848 | RELM-α+ interstitial macrophages inflammatory arthritis synovium |
| ECM1 | 1893 | RELM-α+ interstitial macrophages inflammatory arthritis synovium |
| EHD4 | 30844 | RELM-α+ interstitial macrophages inflammatory arthritis synovium |
| EID1 | 23741 | RELM-α+ interstitial macrophages inflammatory arthritis synovium |
| EPS15 | 2060 | RELM-α+ interstitial macrophages inflammatory arthritis synovium |
| EPS8 | 2059 | RELM-α+ interstitial macrophages inflammatory arthritis synovium |
| ETV1 | 2115 | RELM-α+ interstitial macrophages inflammatory arthritis synovium |
| EVI5 | 7813 | RELM-α+ interstitial macrophages inflammatory arthritis synovium |
| F13A1 | 2162 | RELM-α+ interstitial macrophages inflammatory arthritis synovium |
| FAM46A | 55603 | RELM-α+ interstitial macrophages inflammatory arthritis synovium |
| FCGR2A | 2212 | RELM-α+ interstitial macrophages inflammatory arthritis synovium |
| FCGR2B | 2213 | RELM-α+ interstitial macrophages inflammatory arthritis synovium |
| FCGRT | 2217 | RELM-α+ interstitial macrophages inflammatory arthritis synovium |
| FCHO2 | 115548 | RELM-α+ interstitial macrophages inflammatory arthritis synovium |
| FEZ2 | 9637 | RELM-α+ interstitial macrophages inflammatory arthritis synovium |
| FGFR1 | 2260 | RELM-α+ interstitial macrophages inflammatory arthritis synovium |
| FKBP1A | 2280 | RELM-α+ interstitial macrophages inflammatory arthritis synovium |
| FNTA | 2339 | RELM-α+ interstitial macrophages inflammatory arthritis synovium |
| FOLR2 | 2350 | RELM-α+ interstitial macrophages inflammatory arthritis synovium |
| FOXN3 | 1112 | RELM-α+ interstitial macrophages inflammatory arthritis synovium |
| FOXP1 | 27086 | RELM-α+ interstitial macrophages inflammatory arthritis synovium |
| FRMD4B | 23150 | RELM-α+ interstitial macrophages inflammatory arthritis synovium |
| FXYD2 | 486 | RELM-α+ interstitial macrophages inflammatory arthritis synovium |
| GABARAPL1 | 23710 | RELM-α+ interstitial macrophages inflammatory arthritis synovium |
| GAS6 | 2621 | RELM-α+ interstitial macrophages inflammatory arthritis synovium |
| GAS7 | 8522 | RELM-α+ interstitial macrophages inflammatory arthritis synovium |
| GATM | 2628 | RELM-α+ interstitial macrophages inflammatory arthritis synovium |
| GLTP | 51228 | RELM-α+ interstitial macrophages inflammatory arthritis synovium |
| GLUL | 2752 | RELM-α+ interstitial macrophages inflammatory arthritis synovium |
| GPR34 | 2857 | RELM-α+ interstitial macrophages inflammatory arthritis synovium |
| GSTM5 | 2949 | RELM-α+ interstitial macrophages inflammatory arthritis synovium |
| GYPC | 2995 | RELM-α+ interstitial macrophages inflammatory arthritis synovium |
| H2AFV | 94239 | RELM-α+ interstitial macrophages inflammatory arthritis synovium |
| HACD4 | 401494 | RELM-α+ interstitial macrophages inflammatory arthritis synovium |
| HCST | 10870 | RELM-α+ interstitial macrophages inflammatory arthritis synovium |
| HEXA | 3073 | RELM-α+ interstitial macrophages inflammatory arthritis synovium |
| HFE | 3077 | RELM-α+ interstitial macrophages inflammatory arthritis synovium |
| HIGD2A | 192286 | RELM-α+ interstitial macrophages inflammatory arthritis synovium |
| HIST1H2BI | 8346 | RELM-α+ interstitial macrophages inflammatory arthritis synovium |
| HMGN1 | 3150 | RELM-α+ interstitial macrophages inflammatory arthritis synovium |
| HMOX2 | 3163 | RELM-α+ interstitial macrophages inflammatory arthritis synovium |
| HP1BP3 | 50809 | RELM-α+ interstitial macrophages inflammatory arthritis synovium |
| HPGD | 3248 | RELM-α+ interstitial macrophages inflammatory arthritis synovium |
| HPGDS | 27306 | RELM-α+ interstitial macrophages inflammatory arthritis synovium |
| HPRT1 | 3251 | RELM-α+ interstitial macrophages inflammatory arthritis synovium |
| IDH2 | 3418 | RELM-α+ interstitial macrophages inflammatory arthritis synovium |
| IFI16 | 3428 | RELM-α+ interstitial macrophages inflammatory arthritis synovium |
| IGF1 | 3479 | RELM-α+ interstitial macrophages inflammatory arthritis synovium |
| IGFBP4 | 3487 | RELM-α+ interstitial macrophages inflammatory arthritis synovium |
| IGHM | 3507 | RELM-α+ interstitial macrophages inflammatory arthritis synovium |
| ITM2B | 9445 | RELM-α+ interstitial macrophages inflammatory arthritis synovium |
| ITPRIPL2 | 162073 | RELM-α+ interstitial macrophages inflammatory arthritis synovium |
| ITSN1 | 6453 | RELM-α+ interstitial macrophages inflammatory arthritis synovium |
| KIAA0100 | 9703 | RELM-α+ interstitial macrophages inflammatory arthritis synovium |
| KIF1B | 23095 | RELM-α+ interstitial macrophages inflammatory arthritis synovium |
| LAMP1 | 3916 | RELM-α+ interstitial macrophages inflammatory arthritis synovium |
| LAMP2 | 3920 | RELM-α+ interstitial macrophages inflammatory arthritis synovium |
| LAPTM4A | 9741 | RELM-α+ interstitial macrophages inflammatory arthritis synovium |
| LEPROT | 54741 | RELM-α+ interstitial macrophages inflammatory arthritis synovium |
| LGMN | 5641 | RELM-α+ interstitial macrophages inflammatory arthritis synovium |
| LIFR | 3977 | RELM-α+ interstitial macrophages inflammatory arthritis synovium |
| LIPA | 3988 | RELM-α+ interstitial macrophages inflammatory arthritis synovium |
| LTC4S | 4056 | RELM-α+ interstitial macrophages inflammatory arthritis synovium |
| LY96 | 23643 | RELM-α+ interstitial macrophages inflammatory arthritis synovium |
| LYL1 | 4066 | RELM-α+ interstitial macrophages inflammatory arthritis synovium |
| LYVE1 | 10894 | RELM-α+ interstitial macrophages inflammatory arthritis synovium |
| MAF | 4094 | RELM-α+ interstitial macrophages inflammatory arthritis synovium |
| MAFB | 9935 | RELM-α+ interstitial macrophages inflammatory arthritis synovium |
| MALAT1 | 378938 | RELM-α+ interstitial macrophages inflammatory arthritis synovium |
| MAN1A1 | 4121 | RELM-α+ interstitial macrophages inflammatory arthritis synovium |
| MBNL1 | 4154 | RELM-α+ interstitial macrophages inflammatory arthritis synovium |
| MEF2C | 4208 | RELM-α+ interstitial macrophages inflammatory arthritis synovium |
| MGST3 | 4259 | RELM-α+ interstitial macrophages inflammatory arthritis synovium |
| MID1IP1 | 58526 | RELM-α+ interstitial macrophages inflammatory arthritis synovium |
| MINDY2 | 54629 | RELM-α+ interstitial macrophages inflammatory arthritis synovium |
| MPP1 | 4354 | RELM-α+ interstitial macrophages inflammatory arthritis synovium |
| MRC1 | 4360 | RELM-α+ interstitial macrophages inflammatory arthritis synovium |
| MRFAP1 | 93621 | RELM-α+ interstitial macrophages inflammatory arthritis synovium |
| MTSS1 | 9788 | RELM-α+ interstitial macrophages inflammatory arthritis synovium |
| MYO5A | 4644 | RELM-α+ interstitial macrophages inflammatory arthritis synovium |
| NCOA4 | 8031 | RELM-α+ interstitial macrophages inflammatory arthritis synovium |
| NDFIP1 | 80762 | RELM-α+ interstitial macrophages inflammatory arthritis synovium |
| NDUFS7 | 374291 | RELM-α+ interstitial macrophages inflammatory arthritis synovium |
| NENF | 29937 | RELM-α+ interstitial macrophages inflammatory arthritis synovium |
| NINJ1 | 4814 | RELM-α+ interstitial macrophages inflammatory arthritis synovium |
| NISCH | 11188 | RELM-α+ interstitial macrophages inflammatory arthritis synovium |
| NPL | 80896 | RELM-α+ interstitial macrophages inflammatory arthritis synovium |
| NR3C1 | 2908 | RELM-α+ interstitial macrophages inflammatory arthritis synovium |
| NRP1 | 8829 | RELM-α+ interstitial macrophages inflammatory arthritis synovium |
| NUDT9 | 53343 | RELM-α+ interstitial macrophages inflammatory arthritis synovium |
| OAT | 4942 | RELM-α+ interstitial macrophages inflammatory arthritis synovium |
| P2RY12 | 64805 | RELM-α+ interstitial macrophages inflammatory arthritis synovium |
| P2RY6 | 5031 | RELM-α+ interstitial macrophages inflammatory arthritis synovium |
| PDLIM1 | 9124 | RELM-α+ interstitial macrophages inflammatory arthritis synovium |
| PDLIM4 | 8572 | RELM-α+ interstitial macrophages inflammatory arthritis synovium |
| PEA15 | 8682 | RELM-α+ interstitial macrophages inflammatory arthritis synovium |
| PEPD | 5184 | RELM-α+ interstitial macrophages inflammatory arthritis synovium |
| PF4 | 5196 | RELM-α+ interstitial macrophages inflammatory arthritis synovium |
| PGRMC1 | 10857 | RELM-α+ interstitial macrophages inflammatory arthritis synovium |
| PINK1 | 65018 | RELM-α+ interstitial macrophages inflammatory arthritis synovium |
| PKIG | 11142 | RELM-α+ interstitial macrophages inflammatory arthritis synovium |
| PLTP | 5360 | RELM-α+ interstitial macrophages inflammatory arthritis synovium |
| PMP22 | 5376 | RELM-α+ interstitial macrophages inflammatory arthritis synovium |
| POLR3GL | 84265 | RELM-α+ interstitial macrophages inflammatory arthritis synovium |
| PON2 | 5445 | RELM-α+ interstitial macrophages inflammatory arthritis synovium |
| PPIA | 5478 | RELM-α+ interstitial macrophages inflammatory arthritis synovium |
| PPP3CA | 5530 | RELM-α+ interstitial macrophages inflammatory arthritis synovium |
| PRCP | 5547 | RELM-α+ interstitial macrophages inflammatory arthritis synovium |
| PROS1 | 5627 | RELM-α+ interstitial macrophages inflammatory arthritis synovium |
| PRUNE2 | 158471 | RELM-α+ interstitial macrophages inflammatory arthritis synovium |
| PSD3 | 23362 | RELM-α+ interstitial macrophages inflammatory arthritis synovium |
| PTAFR | 5724 | RELM-α+ interstitial macrophages inflammatory arthritis synovium |
| PTPN18 | 26469 | RELM-α+ interstitial macrophages inflammatory arthritis synovium |
| PTS | 5805 | RELM-α+ interstitial macrophages inflammatory arthritis synovium |
| QPCT | 25797 | RELM-α+ interstitial macrophages inflammatory arthritis synovium |
| RAB11A | 8766 | RELM-α+ interstitial macrophages inflammatory arthritis synovium |
| RAB11FIP5 | 26056 | RELM-α+ interstitial macrophages inflammatory arthritis synovium |
| RAB31 | 11031 | RELM-α+ interstitial macrophages inflammatory arthritis synovium |
| RABAC1 | 10567 | RELM-α+ interstitial macrophages inflammatory arthritis synovium |
| RALBP1 | 10928 | RELM-α+ interstitial macrophages inflammatory arthritis synovium |
| RAPGEF6 | 51735 | RELM-α+ interstitial macrophages inflammatory arthritis synovium |
| RASGRP3 | 25780 | RELM-α+ interstitial macrophages inflammatory arthritis synovium |
| RASSF4 | 83937 | RELM-α+ interstitial macrophages inflammatory arthritis synovium |
| RFK | 55312 | RELM-α+ interstitial macrophages inflammatory arthritis synovium |
| RGL1 | 23179 | RELM-α+ interstitial macrophages inflammatory arthritis synovium |
| RGS10 | 6001 | RELM-α+ interstitial macrophages inflammatory arthritis synovium |
| RGS18 | 64407 | RELM-α+ interstitial macrophages inflammatory arthritis synovium |
| RIN2 | 54453 | RELM-α+ interstitial macrophages inflammatory arthritis synovium |
| RNASE4 | 6038 | RELM-α+ interstitial macrophages inflammatory arthritis synovium |
| RNF13 | 11342 | RELM-α+ interstitial macrophages inflammatory arthritis synovium |
| RNF130 | 55819 | RELM-α+ interstitial macrophages inflammatory arthritis synovium |
| RSRP1 | 57035 | RELM-α+ interstitial macrophages inflammatory arthritis synovium |
| S100A1 | 6271 | RELM-α+ interstitial macrophages inflammatory arthritis synovium |
| S1PR1 | 1901 | RELM-α+ interstitial macrophages inflammatory arthritis synovium |
| SBDS | 51119 | RELM-α+ interstitial macrophages inflammatory arthritis synovium |
| SCAMP2 | 10066 | RELM-α+ interstitial macrophages inflammatory arthritis synovium |
| SDHC | 6391 | RELM-α+ interstitial macrophages inflammatory arthritis synovium |
| SEC14L1 | 6397 | RELM-α+ interstitial macrophages inflammatory arthritis synovium |
| SEC62 | 7095 | RELM-α+ interstitial macrophages inflammatory arthritis synovium |
| SELENBP1 | 8991 | RELM-α+ interstitial macrophages inflammatory arthritis synovium |
| SELENOF | 9403 | RELM-α+ interstitial macrophages inflammatory arthritis synovium |
| SELENOM | 140606 | RELM-α+ interstitial macrophages inflammatory arthritis synovium |
| SELENOP | 6414 | RELM-α+ interstitial macrophages inflammatory arthritis synovium |
| SERINC3 | 10955 | RELM-α+ interstitial macrophages inflammatory arthritis synovium |
| SERPINB1 | 1992 | RELM-α+ interstitial macrophages inflammatory arthritis synovium |
| SERPINB6 | 5269 | RELM-α+ interstitial macrophages inflammatory arthritis synovium |
| SESN1 | 27244 | RELM-α+ interstitial macrophages inflammatory arthritis synovium |
| SGPP1 | 81537 | RELM-α+ interstitial macrophages inflammatory arthritis synovium |
| SH3BGRL | 6451 | RELM-α+ interstitial macrophages inflammatory arthritis synovium |
| SLC29A1 | 2030 | RELM-α+ interstitial macrophages inflammatory arthritis synovium |
| SLC48A1 | 55652 | RELM-α+ interstitial macrophages inflammatory arthritis synovium |
| SLC9A3R2 | 9351 | RELM-α+ interstitial macrophages inflammatory arthritis synovium |
| SLCO2B1 | 11309 | RELM-α+ interstitial macrophages inflammatory arthritis synovium |
| SMAGP | 57228 | RELM-α+ interstitial macrophages inflammatory arthritis synovium |
| SMIM10L1 | 1E+08 | RELM-α+ interstitial macrophages inflammatory arthritis synovium |
| SNX2 | 6643 | RELM-α+ interstitial macrophages inflammatory arthritis synovium |
| SNX3 | 8724 | RELM-α+ interstitial macrophages inflammatory arthritis synovium |
| SNX5 | 27131 | RELM-α+ interstitial macrophages inflammatory arthritis synovium |
| SNX6 | 58533 | RELM-α+ interstitial macrophages inflammatory arthritis synovium |
| SNX8 | 29886 | RELM-α+ interstitial macrophages inflammatory arthritis synovium |
| SPRED1 | 161742 | RELM-α+ interstitial macrophages inflammatory arthritis synovium |
| SPTBN1 | 6711 | RELM-α+ interstitial macrophages inflammatory arthritis synovium |
| SSH2 | 85464 | RELM-α+ interstitial macrophages inflammatory arthritis synovium |
| STAB1 | 23166 | RELM-α+ interstitial macrophages inflammatory arthritis synovium |
| STARD8 | 9754 | RELM-α+ interstitial macrophages inflammatory arthritis synovium |
| STK17B | 9262 | RELM-α+ interstitial macrophages inflammatory arthritis synovium |
| SULF2 | 55959 | RELM-α+ interstitial macrophages inflammatory arthritis synovium |
| SULT1A1 | 6817 | RELM-α+ interstitial macrophages inflammatory arthritis synovium |
| TACC1 | 6867 | RELM-α+ interstitial macrophages inflammatory arthritis synovium |
| TCF4 | 6925 | RELM-α+ interstitial macrophages inflammatory arthritis synovium |
| TCN2 | 6948 | RELM-α+ interstitial macrophages inflammatory arthritis synovium |
| TEP1 | 7011 | RELM-α+ interstitial macrophages inflammatory arthritis synovium |
| TEX264 | 51368 | RELM-α+ interstitial macrophages inflammatory arthritis synovium |
| TF | 7018 | RELM-α+ interstitial macrophages inflammatory arthritis synovium |
| TGFBR2 | 7048 | RELM-α+ interstitial macrophages inflammatory arthritis synovium |
| TIMD4 | 91937 | RELM-α+ interstitial macrophages inflammatory arthritis synovium |
| TIMP2 | 7077 | RELM-α+ interstitial macrophages inflammatory arthritis synovium |
| TLR7 | 51284 | RELM-α+ interstitial macrophages inflammatory arthritis synovium |
| TMEM106A | 113277 | RELM-α+ interstitial macrophages inflammatory arthritis synovium |
| TMEM141 | 85014 | RELM-α+ interstitial macrophages inflammatory arthritis synovium |
| TMEM37 | 140738 | RELM-α+ interstitial macrophages inflammatory arthritis synovium |
| TMEM50A | 23585 | RELM-α+ interstitial macrophages inflammatory arthritis synovium |
| TMEM59 | 9528 | RELM-α+ interstitial macrophages inflammatory arthritis synovium |
| TNFAIP8L2 | 79626 | RELM-α+ interstitial macrophages inflammatory arthritis synovium |
| TNFSF12 | 8742 | RELM-α+ interstitial macrophages inflammatory arthritis synovium |
| TNS1 | 7145 | RELM-α+ interstitial macrophages inflammatory arthritis synovium |
| TPPP3 | 51673 | RELM-α+ interstitial macrophages inflammatory arthritis synovium |
| TPRG1L | 127262 | RELM-α+ interstitial macrophages inflammatory arthritis synovium |
| TRIM47 | 91107 | RELM-α+ interstitial macrophages inflammatory arthritis synovium |
| TRMT1 | 55621 | RELM-α+ interstitial macrophages inflammatory arthritis synovium |
| TSC22D3 | 1831 | RELM-α+ interstitial macrophages inflammatory arthritis synovium |
| TSEN34 | 79042 | RELM-α+ interstitial macrophages inflammatory arthritis synovium |
| TSLP | 85480 | RELM-α+ interstitial macrophages inflammatory arthritis synovium |
| TSPAN17 | 26262 | RELM-α+ interstitial macrophages inflammatory arthritis synovium |
| TSPAN4 | 7106 | RELM-α+ interstitial macrophages inflammatory arthritis synovium |
| TXNIP | 10628 | RELM-α+ interstitial macrophages inflammatory arthritis synovium |
| UNC93B1 | 81622 | RELM-α+ interstitial macrophages inflammatory arthritis synovium |
| VAT1 | 10493 | RELM-α+ interstitial macrophages inflammatory arthritis synovium |
| VKORC1 | 79001 | RELM-α+ interstitial macrophages inflammatory arthritis synovium |
| WWP1 | 11059 | RELM-α+ interstitial macrophages inflammatory arthritis synovium |
| YPEL3 | 83719 | RELM-α+ interstitial macrophages inflammatory arthritis synovium |
| ZBTB20 | 26137 | RELM-α+ interstitial macrophages inflammatory arthritis synovium |
| ZCCHC6 | 79670 | RELM-α+ interstitial macrophages inflammatory arthritis synovium |
| ALOX5 | 240 | MHCII+ interstitial macrophages inflammatory arthritis synovium |
| AP2A2 | 161 | MHCII+ interstitial macrophages inflammatory arthritis synovium |
| AQP1 | 358 | MHCII+ interstitial macrophages inflammatory arthritis synovium |
| ATF3 | 467 | MHCII+ interstitial macrophages inflammatory arthritis synovium |
| ATP2B1 | 490 | MHCII+ interstitial macrophages inflammatory arthritis synovium |
| CCL24 | 6369 | MHCII+ interstitial macrophages inflammatory arthritis synovium |
| CD163 | 9332 | MHCII+ interstitial macrophages inflammatory arthritis synovium |
| CD36 | 948 | MHCII+ interstitial macrophages inflammatory arthritis synovium |
| CD81 | 975 | MHCII+ interstitial macrophages inflammatory arthritis synovium |
| CD93 | 22918 | MHCII+ interstitial macrophages inflammatory arthritis synovium |
| CLEC10A | 10462 | MHCII+ interstitial macrophages inflammatory arthritis synovium |
| CLEC12A | 160364 | MHCII+ interstitial macrophages inflammatory arthritis synovium |
| CLTC | 1213 | MHCII+ interstitial macrophages inflammatory arthritis synovium |
| DAB2 | 1601 | MHCII+ interstitial macrophages inflammatory arthritis synovium |
| DOK2 | 9046 | MHCII+ interstitial macrophages inflammatory arthritis synovium |
| DSE | 29940 | MHCII+ interstitial macrophages inflammatory arthritis synovium |
| EGR1 | 1958 | MHCII+ interstitial macrophages inflammatory arthritis synovium |
| EHD4 | 30844 | MHCII+ interstitial macrophages inflammatory arthritis synovium |
| FOSB | 2354 | MHCII+ interstitial macrophages inflammatory arthritis synovium |
| FRMD4B | 23150 | MHCII+ interstitial macrophages inflammatory arthritis synovium |
| FXYD2 | 486 | MHCII+ interstitial macrophages inflammatory arthritis synovium |
| GAS7 | 8522 | MHCII+ interstitial macrophages inflammatory arthritis synovium |
| HLA-DQA1 | 3117 | MHCII+ interstitial macrophages inflammatory arthritis synovium |
| HLA-DQB1 | 3119 | MHCII+ interstitial macrophages inflammatory arthritis synovium |
| HLA-DRB5 | 3127 | MHCII+ interstitial macrophages inflammatory arthritis synovium |
| HSPA1A | 3303 | MHCII+ interstitial macrophages inflammatory arthritis synovium |
| IER2 | 9592 | MHCII+ interstitial macrophages inflammatory arthritis synovium |
| IGF1 | 3479 | MHCII+ interstitial macrophages inflammatory arthritis synovium |
| IGFBP4 | 3487 | MHCII+ interstitial macrophages inflammatory arthritis synovium |
| IGHM | 3507 | MHCII+ interstitial macrophages inflammatory arthritis synovium |
| IRF2BP2 | 359948 | MHCII+ interstitial macrophages inflammatory arthritis synovium |
| JUN | 3725 | MHCII+ interstitial macrophages inflammatory arthritis synovium |
| JUND | 3727 | MHCII+ interstitial macrophages inflammatory arthritis synovium |
| KLF2 | 10365 | MHCII+ interstitial macrophages inflammatory arthritis synovium |
| KLF4 | 9314 | MHCII+ interstitial macrophages inflammatory arthritis synovium |
| KLF6 | 1316 | MHCII+ interstitial macrophages inflammatory arthritis synovium |
| LIFR | 3977 | MHCII+ interstitial macrophages inflammatory arthritis synovium |
| LPL | 4023 | MHCII+ interstitial macrophages inflammatory arthritis synovium |
| LYZ | 4069 | MHCII+ interstitial macrophages inflammatory arthritis synovium |
| MAF | 4094 | MHCII+ interstitial macrophages inflammatory arthritis synovium |
| MAFB | 9935 | MHCII+ interstitial macrophages inflammatory arthritis synovium |
| MALAT1 | 378938 | MHCII+ interstitial macrophages inflammatory arthritis synovium |
| MARCKSL1 | 65108 | MHCII+ interstitial macrophages inflammatory arthritis synovium |
| MEF2C | 4208 | MHCII+ interstitial macrophages inflammatory arthritis synovium |
| METRNL | 284207 | MHCII+ interstitial macrophages inflammatory arthritis synovium |
| MRC1 | 4360 | MHCII+ interstitial macrophages inflammatory arthritis synovium |
| MS4A7 | 58475 | MHCII+ interstitial macrophages inflammatory arthritis synovium |
| MT-CO1 | 4512 | MHCII+ interstitial macrophages inflammatory arthritis synovium |
| MT1A | 4489 | MHCII+ interstitial macrophages inflammatory arthritis synovium |
| MT2A | 4502 | MHCII+ interstitial macrophages inflammatory arthritis synovium |
| MTSS1 | 9788 | MHCII+ interstitial macrophages inflammatory arthritis synovium |
| NFKBIZ | 64332 | MHCII+ interstitial macrophages inflammatory arthritis synovium |
| PF4 | 5196 | MHCII+ interstitial macrophages inflammatory arthritis synovium |
| PPP3CA | 5530 | MHCII+ interstitial macrophages inflammatory arthritis synovium |
| RAB7B | 338382 | MHCII+ interstitial macrophages inflammatory arthritis synovium |
| RBPJ | 3516 | MHCII+ interstitial macrophages inflammatory arthritis synovium |
| RND3 | 390 | MHCII+ interstitial macrophages inflammatory arthritis synovium |
| RTN4 | 57142 | MHCII+ interstitial macrophages inflammatory arthritis synovium |
| SDC3 | 9672 | MHCII+ interstitial macrophages inflammatory arthritis synovium |
| SELENOP | 6414 | MHCII+ interstitial macrophages inflammatory arthritis synovium |
| SERINC3 | 10955 | MHCII+ interstitial macrophages inflammatory arthritis synovium |
| SLAMF9 | 89886 | MHCII+ interstitial macrophages inflammatory arthritis synovium |
| SNX5 | 27131 | MHCII+ interstitial macrophages inflammatory arthritis synovium |
| TMEM176A | 55365 | MHCII+ interstitial macrophages inflammatory arthritis synovium |
| TMEM176B | 28959 | MHCII+ interstitial macrophages inflammatory arthritis synovium |
| TUBB2A | 7280 | MHCII+ interstitial macrophages inflammatory arthritis synovium |
| TXNIP | 10628 | MHCII+ interstitial macrophages inflammatory arthritis synovium |
| WNK1 | 65125 | MHCII+ interstitial macrophages inflammatory arthritis synovium |
| WWP1 | 11059 | MHCII+ interstitial macrophages inflammatory arthritis synovium |
| ZFP36L1 | 677 | MHCII+ interstitial macrophages inflammatory arthritis synovium |
| ACOT7 | 11332 | CCR2+ARG1+ infiltrating macrophages inflammatory arthritis |
| | | synovium |
| ACP5 | 54 | CCR2+ARG1+ infiltrating macrophages inflammatory arthritis |
| | | synovium |
| ACTR2 | 10097 | CCR2+ARG1+ infiltrating macrophages inflammatory arthritis |
| | | synovium |
| ADAM8 | 101 | CCR2+ARG1+ infiltrating macrophages inflammatory arthritis |
| | | synovium |
| ADSSL1 | 122622 | CCR2+ARG1+ infiltrating macrophages inflammatory arthritis |
| | | synovium |
| AGPAT4 | 56895 | CCR2+ARG1+ infiltrating macrophages inflammatory arthritis |
| | | synovium |
| AGPAT5 | 55326 | CCR2+ARG1+ infiltrating macrophages inflammatory arthritis |
| | | synovium |
| AK2 | 204 | CCR2+ARG1+ infiltrating macrophages inflammatory arthritis |
| | | synovium |
| AK6 | 1.02E+08 | CCR2+ARG1+ infiltrating macrophages inflammatory arthritis |
| | | synovium |
| ALDOA | 226 | CCR2+ARG1+ infiltrating macrophages inflammatory arthritis |
| | | synovium |
| ANP32B | 10541 | CCR2+ARG1+ infiltrating macrophages inflammatory arthritis |
| | | synovium |
| ANXA1 | 301 | CCR2+ARG1+ infiltrating macrophages inflammatory arthritis |
| | | synovium |
| ANXA2 | 302 | CCR2+ARG1+ infiltrating macrophages inflammatory arthritis |
| | | synovium |
| ANXA5 | 308 | CCR2+ARG1+ infiltrating macrophages inflammatory arthritis |
| | | synovium |
| APEX1 | 328 | CCR2+ARG1+ infiltrating macrophages inflammatory arthritis |
| | | synovium |
| APOBEC1 | 339 | CCR2+ARG1+ infiltrating macrophages inflammatory arthritis |
| | | synovium |
| APRT | 353 | CCR2+ARG1+ infiltrating macrophages inflammatory arthritis |
| | | synovium |
| ARF6 | 382 | CCR2+ARG1+ infiltrating macrophages inflammatory arthritis |
| | | synovium |
| ATOX1 | 475 | CCR2+ARG1+ infiltrating macrophages inflammatory arthritis |
| | | synovium |
| ATP5F1E | 514 | CCR2+ARG1+ infiltrating macrophages inflammatory arthritis |
| | | synovium |
| ATP5G1 | 516 | CCR2+ARG1+ infiltrating macrophages inflammatory arthritis |
| | | synovium |
| ATP5G3 | 518 | CCR2+ARG1+ infiltrating macrophages inflammatory arthritis |
| | | synovium |
| ATP5L | 10632 | CCR2+ARG1+ infiltrating macrophages inflammatory arthritis |
| | | synovium |
| ATP5ME | 521 | CCR2+ARG1+ infiltrating macrophages inflammatory arthritis |
| | | synovium |
| ATP5O | 539 | CCR2+ARG1+ infiltrating macrophages inflammatory arthritis |
| | | synovium |
| ATP6V0E1 | 8992 | CCR2+ARG1+ infiltrating macrophages inflammatory arthritis |
| | | synovium |
| ATP6V1A | 523 | CCR2+ARG1+ infiltrating macrophages inflammatory arthritis |
| | | synovium |
| ATP6V1B2 | 526 | CCR2+ARG1+ infiltrating macrophages inflammatory arthritis |
| | | synovium |
| ATP6V1C1 | 528 | CCR2+ARG1+ infiltrating macrophages inflammatory arthritis |
| | | synovium |
| ATP6V1D | 51382 | CCR2+ARG1+ infiltrating macrophages inflammatory arthritis |
| | | synovium |
| ATP6V1E1 | 529 | CCR2+ARG1+ infiltrating macrophages inflammatory arthritis |
| | | synovium |
| ATP6V1G1 | 9550 | CCR2+ARG1+ infiltrating macrophages inflammatory arthritis |
| | | synovium |
| ATPIF1 | 93974 | CCR2+ARG1+ infiltrating macrophages inflammatory arthritis |
| | | synovium |
| AVPI1 | 60370 | CCR2+ARG1+ infiltrating macrophages inflammatory arthritis |
| | | synovium |
| AZIN1 | 51582 | CCR2+ARG1+ infiltrating macrophages inflammatory arthritis |
| | | synovium |
| B4GALT1 | 2683 | CCR2+ARG1+ infiltrating macrophages inflammatory arthritis |
| | | synovium |
| BANF1 | 8815 | CCR2+ARG1+ infiltrating macrophages inflammatory arthritis |
| | | synovium |
| BAX | 581 | CCR2+ARG1+ infiltrating macrophages inflammatory arthritis |
| | | synovium |
| BDH2 | 56898 | CCR2+ARG1+ infiltrating macrophages inflammatory arthritis |
| | | synovium |
| BHLHE40 | 8553 | CCR2+ARG1+ infiltrating macrophages inflammatory arthritis |
| | | synovium |
| BOLA2 | 552900 | CCR2+ARG1+ infiltrating macrophages inflammatory arthritis |
| | | synovium |
| BTG1 | 694 | CCR2+ARG1+ infiltrating macrophages inflammatory arthritis |
| | | synovium |
| C14orf2 | 9556 | CCR2+ARG1+ infiltrating macrophages inflammatory arthritis |
| | | synovium |
| C1QBP | 708 | CCR2+ARG1+ infiltrating macrophages inflammatory arthritis |
| | | synovium |
| C6orf62 | 81688 | CCR2+ARG1+ infiltrating macrophages inflammatory arthritis |
| | | synovium |
| C8orf59 | 401466 | CCR2+ARG1+ infiltrating macrophages inflammatory arthritis |
| | | synovium |
| CALM1 | 801 | CCR2+ARG1+ infiltrating macrophages inflammatory arthritis |
| | | synovium |
| CAV1 | 857 | CCR2+ARG1+ infiltrating macrophages inflammatory arthritis |
| | | synovium |
| CAV2 | 858 | CCR2+ARG1+ infiltrating macrophages inflammatory arthritis |
| | | synovium |
| CCT2 | 10576 | CCR2+ARG1+ infiltrating macrophages inflammatory arthritis |
| | | synovium |
| CCT3 | 7203 | CCR2+ARG1+ infiltrating macrophages inflammatory arthritis |
| | | synovium |
| CCT8 | 10694 | CCR2+ARG1+ infiltrating macrophages inflammatory arthritis |
| | | synovium |
| CD44 | 960 | CCR2+ARG1+ infiltrating macrophages inflammatory arthritis |
| | | synovium |
| CHCHD2 | 51142 | CCR2+ARG1+ infiltrating macrophages inflammatory arthritis |
| | | synovium |
| CHCHD4 | 131474 | CCR2+ARG1+ infiltrating macrophages inflammatory arthritis |
| | | synovium |
| CITED2 | 10370 | CCR2+ARG1+ infiltrating macrophages inflammatory arthritis |
| | | synovium |
| CKAP4 | 10970 | CCR2+ARG1+ infiltrating macrophages inflammatory arthritis |
| | | synovium |
| CLEC4D | 338339 | CCR2+ARG1+ infiltrating macrophages inflammatory arthritis |
| | | synovium |
| CLEC4E | 26253 | CCR2+ARG1+ infiltrating macrophages inflammatory arthritis |
| | | synovium |
| CLEC6A | 93978 | CCR2+ARG1+ infiltrating macrophages inflammatory arthritis |
| | | synovium |
| COPS9 | 150678 | CCR2+ARG1+ infiltrating macrophages inflammatory arthritis |
| | | synovium |
| COX17 | 10063 | CCR2+ARG1+ infiltrating macrophages inflammatory arthritis |
| | | synovium |
| COX5A | 9377 | CCR2+ARG1+ infiltrating macrophages inflammatory arthritis |
| | | synovium |
| COX6B1 | 1340 | CCR2+ARG1+ infiltrating macrophages inflammatory arthritis |
| | | synovium |
| COX7A2 | 1347 | CCR2+ARG1+ infiltrating macrophages inflammatory arthritis |
| | | synovium |
| COX7B | 1349 | CCR2+ARG1+ infiltrating macrophages inflammatory arthritis |
| | | synovium |
| COX7C | 1350 | CCR2+ARG1+ infiltrating macrophages inflammatory arthritis |
| | | synovium |
| CSTB | 1476 | CCR2+ARG1+ infiltrating macrophages inflammatory arthritis |
| | | synovium |
| CTSK | 1513 | CCR2+ARG1+ infiltrating macrophages inflammatory arthritis |
| | | synovium |
| CTSV | 1515 | CCR2+ARG1+ infiltrating macrophages inflammatory arthritis |
| | | synovium |
| CXCL16 | 58191 | CCR2+ARG1+ infiltrating macrophages inflammatory arthritis |
| | | synovium |
| CXCL3 | 2921 | CCR2+ARG1+ infiltrating macrophages inflammatory arthritis |
| | | synovium |
| CYCS | 54205 | CCR2+ARG1+ infiltrating macrophages inflammatory arthritis |
| | | synovium |
| DDT | 1652 | CCR2+ARG1+ infiltrating macrophages inflammatory arthritis |
| | | synovium |
| DNAJC2 | 27000 | CCR2+ARG1+ infiltrating macrophages inflammatory arthritis |
| | | synovium |
| DTYMK | 1841 | CCR2+ARG1+ infiltrating macrophages inflammatory arthritis |
| | | synovium |
| EBNA1BP2 | 10969 | CCR2+ARG1+ infiltrating macrophages inflammatory arthritis |
| | | synovium |
| EEF1E1 | 9521 | CCR2+ARG1+ infiltrating macrophages inflammatory arthritis |
| | | synovium |
| EEF1G | 1937 | CCR2+ARG1+ infiltrating macrophages inflammatory arthritis |
| | | synovium |
| EFHD2 | 79180 | CCR2+ARG1+ infiltrating macrophages inflammatory arthritis |
| | | synovium |
| EHD1 | 10938 | CCR2+ARG1+ infiltrating macrophages inflammatory arthritis |
| | | synovium |
| EIF1AY | 9086 | CCR2+ARG1+ infiltrating macrophages inflammatory arthritis |
| | | synovium |
| EIF2S1 | 1965 | CCR2+ARG1+ infiltrating macrophages inflammatory arthritis |
| | | synovium |
| EIF2S2 | 8894 | CCR2+ARG1+ infiltrating macrophages inflammatory arthritis |
| | | synovium |
| EIF3J | 8669 | CCR2+ARG1+ infiltrating macrophages inflammatory arthritis |
| | | synovium |
| EIF4A1 | 1973 | CCR2+ARG1+ infiltrating macrophages inflammatory arthritis |
| | | synovium |
| EIF4G2 | 1982 | CCR2+ARG1+ infiltrating macrophages inflammatory arthritis |
| | | synovium |
| EIF5A | 1984 | CCR2+ARG1+ infiltrating macrophages inflammatory arthritis |
| | | synovium |
| EIF6 | 3692 | CCR2+ARG1+ infiltrating macrophages inflammatory arthritis |
| | | synovium |
| EMC6 | 83460 | CCR2+ARG1+ infiltrating macrophages inflammatory arthritis |
| | | synovium |
| ENO1 | 2023 | CCR2+ARG1+ infiltrating macrophages inflammatory arthritis |
| | | synovium |
| ERH | 2079 | CCR2+ARG1+ infiltrating macrophages inflammatory arthritis |
| | | synovium |
| ESD | 2098 | CCR2+ARG1+ infiltrating macrophages inflammatory arthritis |
| | | synovium |
| EZR | 7430 | CCR2+ARG1+ infiltrating macrophages inflammatory arthritis |
| | | synovium |
| FABP5 | 2171 | CCR2+ARG1+ infiltrating macrophages inflammatory arthritis |
| | | synovium |
| FAM129B | 64855 | CCR2+ARG1+ infiltrating macrophages inflammatory arthritis |
| | | synovium |
| FAM162A | 26355 | CCR2+ARG1+ infiltrating macrophages inflammatory arthritis |
| | | synovium |
| FBL | 2091 | CCR2+ARG1+ infiltrating macrophages inflammatory arthritis |
| | | synovium |
| FCF1 | 51077 | CCR2+ARG1+ infiltrating macrophages inflammatory arthritis |
| | | synovium |
| FN1 | 2335 | CCR2+ARG1+ infiltrating macrophages inflammatory arthritis |
| | | synovium |
| FURIN | 5045 | CCR2+ARG1+ infiltrating macrophages inflammatory arthritis |
| | | synovium |
| G3BP1 | 10146 | CCR2+ARG1+ infiltrating macrophages inflammatory arthritis |
| | | synovium |
| GAPDH | 2597 | CCR2+ARG1+ infiltrating macrophages inflammatory arthritis |
| | | synovium |
| GCSH | 2653 | CCR2+ARG1+ infiltrating macrophages inflammatory arthritis |
| | | synovium |
| GLRX | 2745 | CCR2+ARG1+ infiltrating macrophages inflammatory arthritis |
| | | synovium |
| GNA13 | 10672 | CCR2+ARG1+ infiltrating macrophages inflammatory arthritis |
| | | synovium |
| GNGT2 | 2793 | CCR2+ARG1+ infiltrating macrophages inflammatory arthritis |
| | | synovium |
| GNL3 | 26354 | CCR2+ARG1+ infiltrating macrophages inflammatory arthritis |
| | | synovium |
| GPR137B | 7107 | CCR2+ARG1+ infiltrating macrophages inflammatory arthritis |
| | | synovium |
| HDGF | 3068 | CCR2+ARG1+ infiltrating macrophages inflammatory arthritis |
| | | synovium |
| HIF1A | 3091 | CCR2+ARG1+ infiltrating macrophages inflammatory arthritis |
| | | synovium |
| HIGD1A | 25994 | CCR2+ARG1+ infiltrating macrophages inflammatory arthritis |
| | | synovium |
| HILPDA | 29923 | CCR2+ARG1+ infiltrating macrophages inflammatory arthritis |
| | | synovium |
| HNRNPA0 | 10949 | CCR2+ARG1+ infiltrating macrophages inflammatory arthritis |
| | | synovium |
| HNRNPA1 | 3178 | CCR2+ARG1+ infiltrating macrophages inflammatory arthritis |
| | | synovium |
| HNRNPAB | 3182 | CCR2+ARG1+ infiltrating macrophages inflammatory arthritis |
| | | synovium |
| HSP90AA1 | 3320 | CCR2+ARG1+ infiltrating macrophages inflammatory arthritis |
| | | synovium |
| HSP90AB1 | 3326 | CCR2+ARG1+ infiltrating macrophages inflammatory arthritis |
| | | synovium |
| HSPA9 | 3313 | CCR2+ARG1+ infiltrating macrophages inflammatory arthritis |
| | | synovium |
| HSPD1 | 3329 | CCR2+ARG1+ infiltrating macrophages inflammatory arthritis |
| | | synovium |
| HSPE1 | 3336 | CCR2+ARG1+ infiltrating macrophages inflammatory arthritis |
| | | synovium |
| ID2 | 3398 | CCR2+ARG1+ infiltrating macrophages inflammatory arthritis |
| | | synovium |
| IFRD1 | 3475 | CCR2+ARG1+ infiltrating macrophages inflammatory arthritis |
| | | synovium |
| IL1RN | 3557 | CCR2+ARG1+ infiltrating macrophages inflammatory arthritis |
| | | synovium |
| IMPDH2 | 3615 | CCR2+ARG1+ infiltrating macrophages inflammatory arthritis |
| | | synovium |
| ITGA5 | 3678 | CCR2+ARG1+ infiltrating macrophages inflammatory arthritis |
| | | synovium |
| ITGAV | 3685 | CCR2+ARG1+ infiltrating macrophages inflammatory arthritis |
| | | synovium |
| JARID2 | 3720 | CCR2+ARG1+ infiltrating macrophages inflammatory arthritis |
| | | synovium |
| KCNN4 | 3783 | CCR2+ARG1+ infiltrating macrophages inflammatory arthritis |
| | | synovium |
| LASP1 | 3927 | CCR2+ARG1+ infiltrating macrophages inflammatory arthritis |
| | | synovium |
| LDHA | 3939 | CCR2+ARG1+ infiltrating macrophages inflammatory arthritis |
| | | synovium |
| LFNG | 3955 | CCR2+ARG1+ infiltrating macrophages inflammatory arthritis |
| | | synovium |
| LGALS1 | 3956 | CCR2+ARG1+ infiltrating macrophages inflammatory arthritis |
| | | synovium |
| LGALS3 | 3958 | CCR2+ARG1+ infiltrating macrophages inflammatory arthritis |
| | | synovium |
| LLPH | 84298 | CCR2+ARG1+ infiltrating macrophages inflammatory arthritis |
| | | synovium |
| LMNA | 4000 | CCR2+ARG1+ infiltrating macrophages inflammatory arthritis |
| | | synovium |
| LOC102724828 | 1.03E+08 | CCR2+ARG1+ infiltrating macrophages inflammatory arthritis |
| | | synovium |
| LPL | 4023 | CCR2+ARG1+ infiltrating macrophages inflammatory arthritis |
| | | synovium |
| LSM7 | 51690 | CCR2+ARG1+ infiltrating macrophages inflammatory arthritis |
| | | synovium |
| M6PR | 4074 | CCR2+ARG1+ infiltrating macrophages inflammatory arthritis |
| | | synovium |
| MAGOH | 4116 | CCR2+ARG1+ infiltrating macrophages inflammatory arthritis |
| | | synovium |
| MALT1 | 10892 | CCR2+ARG1+ infiltrating macrophages inflammatory arthritis |
| | | synovium |
| MAP4K4 | 9448 | CCR2+ARG1+ infiltrating macrophages inflammatory arthritis |
| | | synovium |
| MCL1 | 4170 | CCR2+ARG1+ infiltrating macrophages inflammatory arthritis |
| | | synovium |
| MDH2 | 4191 | CCR2+ARG1+ infiltrating macrophages inflammatory arthritis |
| | | synovium |
| MDM2 | 4193 | CCR2+ARG1+ infiltrating macrophages inflammatory arthritis |
| | | synovium |
| MIF | 4282 | CCR2+ARG1+ infiltrating macrophages inflammatory arthritis |
| | | synovium |
| MMP14 | 4323 | CCR2+ARG1+ infiltrating macrophages inflammatory arthritis |
| | | synovium |
| MMP19 | 4327 | CCR2+ARG1+ infiltrating macrophages inflammatory arthritis |
| | | synovium |
| MRPL12 | 6182 | CCR2+ARG1+ infiltrating macrophages inflammatory arthritis |
| | | synovium |
| MRPL20 | 55052 | CCR2+ARG1+ infiltrating macrophages inflammatory arthritis |
| | | synovium |
| MRPL52 | 122704 | CCR2+ARG1+ infiltrating macrophages inflammatory arthritis |
| | | synovium |
| MRPL54 | 116541 | CCR2+ARG1+ infiltrating macrophages inflammatory arthritis |
| | | synovium |
| MRPS14 | 63931 | CCR2+ARG1+ infiltrating macrophages inflammatory arthritis |
| | | synovium |
| MRPS28 | 28957 | CCR2+ARG1+ infiltrating macrophages inflammatory arthritis |
| | | synovium |
| MRTO4 | 51154 | CCR2+ARG1+ infiltrating macrophages inflammatory arthritis |
| | | synovium |
| MS4A7 | 58475 | CCR2+ARG1+ infiltrating macrophages inflammatory arthritis |
| | | synovium |
| MT-ATP8 | 4509 | CCR2+ARG1+ infiltrating macrophages inflammatory arthritis |
| | | synovium |
| MT-CYB | 4519 | CCR2+ARG1+ infiltrating macrophages inflammatory arthritis |
| | | synovium |
| MT-ND1 | 4535 | CCR2+ARG1+ infiltrating macrophages inflammatory arthritis |
| | | synovium |
| MT-ND3 | 4537 | CCR2+ARG1+ infiltrating macrophages inflammatory arthritis |
| | | synovium |
| NAA50 | 80218 | CCR2+ARG1+ infiltrating macrophages inflammatory arthritis |
| | | synovium |
| NCEH1 | 57552 | CCR2+ARG1+ infiltrating macrophages inflammatory arthritis |
| | | synovium |
| NCL | 4691 | CCR2+ARG1+ infiltrating macrophages inflammatory arthritis |
| | | synovium |
| NDUFAB1 | 4706 | CCR2+ARG1+ infiltrating macrophages inflammatory arthritis |
| | | synovium |
| NDUFB6 | 4712 | CCR2+ARG1+ infiltrating macrophages inflammatory arthritis |
| | | synovium |
| NDUFC1 | 4717 | CCR2+ARG1+ infiltrating macrophages inflammatory arthritis |
| | | synovium |
| NDUFC2 | 4718 | CCR2+ARG1+ infiltrating macrophages inflammatory arthritis |
| | | synovium |
| NDUFS6 | 4726 | CCR2+ARG1+ infiltrating macrophages inflammatory arthritis |
| | | synovium |
| NFATC1 | 4772 | CCR2+ARG1+ infiltrating macrophages inflammatory arthritis |
| | | synovium |
| NFKBIA | 4792 | CCR2+ARG1+ infiltrating macrophages inflammatory arthritis |
| | | synovium |
| NHP2 | 55651 | CCR2+ARG1+ infiltrating macrophages inflammatory arthritis |
| | | synovium |
| NIP7 | 51388 | CCR2+ARG1+ infiltrating macrophages inflammatory arthritis |
| | | synovium |
| NME1 | 4830 | CCR2+ARG1+ infiltrating macrophages inflammatory arthritis |
| | | synovium |
| NOLC1 | 9221 | CCR2+ARG1+ infiltrating macrophages inflammatory arthritis |
| | | synovium |
| NOP10 | 55505 | CCR2+ARG1+ infiltrating macrophages inflammatory arthritis |
| | | synovium |
| NOP56 | 10528 | CCR2+ARG1+ infiltrating macrophages inflammatory arthritis |
| | | synovium |
| NOP58 | 51602 | CCR2+ARG1+ infiltrating macrophages inflammatory arthritis |
| | | synovium |
| NPM1 | 4869 | CCR2+ARG1+ infiltrating macrophages inflammatory arthritis |
| | | synovium |
| NPM3 | 10360 | CCR2+ARG1+ infiltrating macrophages inflammatory arthritis |
| | | synovium |
| NRP2 | 8828 | CCR2+ARG1+ infiltrating macrophages inflammatory arthritis |
| | | synovium |
| NSD2 | 7468 | CCR2+ARG1+ infiltrating macrophages inflammatory arthritis |
| | | synovium |
| NSUN2 | 54888 | CCR2+ARG1+ infiltrating macrophages inflammatory arthritis |
| | | synovium |
| NUDCD2 | 134492 | CCR2+ARG1+ infiltrating macrophages inflammatory arthritis |
| | | synovium |
| NUS1 | 116150 | CCR2+ARG1+ infiltrating macrophages inflammatory arthritis |
| | | synovium |
| ODC1 | 4953 | CCR2+ARG1+ infiltrating macrophages inflammatory arthritis |
| | | synovium |
| OSBPL8 | 114882 | CCR2+ARG1+ infiltrating macrophages inflammatory arthritis |
| | | synovium |
| PA2G4 | 5036 | CCR2+ARG1+ infiltrating macrophages inflammatory arthritis |
| | | synovium |
| PCBP1 | 5093 | CCR2+ARG1+ infiltrating macrophages inflammatory arthritis |
| | | synovium |
| PDPN | 10630 | CCR2+ARG1+ infiltrating macrophages inflammatory arthritis |
| | | synovium |
| PFDN4 | 5203 | CCR2+ARG1+ infiltrating macrophages inflammatory arthritis |
| | | synovium |
| PFN1 | 5216 | CCR2+ARG1+ infiltrating macrophages inflammatory arthritis |
| | | synovium |
| PGAM1 | 5223 | CCR2+ARG1+ infiltrating macrophages inflammatory arthritis |
| | | synovium |
| PGK1 | 5230 | CCR2+ARG1+ infiltrating macrophages inflammatory arthritis |
| | | synovium |
| PHB2 | 11331 | CCR2+ARG1+ infiltrating macrophages inflammatory arthritis |
| | | synovium |
| PIK3R5 | 23533 | CCR2+ARG1+ infiltrating macrophages inflammatory arthritis |
| | | synovium |
| PKM | 5315 | CCR2+ARG1+ infiltrating macrophages inflammatory arthritis |
| | | synovium |
| PLIN2 | 123 | CCR2+ARG1+ infiltrating macrophages inflammatory arthritis |
| | | synovium |
| PLXND1 | 23129 | CCR2+ARG1+ infiltrating macrophages inflammatory arthritis |
| | | synovium |
| PNO1 | 56902 | CCR2+ARG1+ infiltrating macrophages inflammatory arthritis |
| | | synovium |
| POLR2K | 5440 | CCR2+ARG1+ infiltrating macrophages inflammatory arthritis |
| | | synovium |
| POLR2L | 5441 | CCR2+ARG1+ infiltrating macrophages inflammatory arthritis |
| | | synovium |
| PPA1 | 5464 | CCR2+ARG1+ infiltrating macrophages inflammatory arthritis |
| | | synovium |
| PPP2CA | 5515 | CCR2+ARG1+ infiltrating macrophages inflammatory arthritis |
| | | synovium |
| PRMT1 | 3276 | CCR2+ARG1+ infiltrating macrophages inflammatory arthritis |
| | | synovium |
| PSMA3 | 5684 | CCR2+ARG1+ infiltrating macrophages inflammatory arthritis |
| | | synovium |
| PSMA6 | 5687 | CCR2+ARG1+ infiltrating macrophages inflammatory arthritis |
| | | synovium |
| PSMB5 | 5693 | CCR2+ARG1+ infiltrating macrophages inflammatory arthritis |
| | | synovium |
| PSMB6 | 5694 | CCR2+ARG1+ infiltrating macrophages inflammatory arthritis |
| | | synovium |
| PSMB7 | 5695 | CCR2+ARG1+ infiltrating macrophages inflammatory arthritis |
| | | synovium |
| PSMD14 | 10213 | CCR2+ARG1+ infiltrating macrophages inflammatory arthritis |
| | | synovium |
| PTBP3 | 9991 | CCR2+ARG1+ infiltrating macrophages inflammatory arthritis |
| | | synovium |
| PTMA | 5757 | CCR2+ARG1+ infiltrating macrophages inflammatory arthritis |
| | | synovium |
| RACK1 | 10399 | CCR2+ARG1+ infiltrating macrophages inflammatory arthritis |
| | | synovium |
| RAI14 | 26064 | CCR2+ARG1+ infiltrating macrophages inflammatory arthritis |
| | | synovium |
| RALA | 5898 | CCR2+ARG1+ infiltrating macrophages inflammatory arthritis |
| | | synovium |
| RAN | 5901 | CCR2+ARG1+ infiltrating macrophages inflammatory arthritis |
| | | synovium |
| RANBP1 | 5902 | CCR2+ARG1+ infiltrating macrophages inflammatory arthritis |
| | | synovium |
| RBM3 | 5935 | CCR2+ARG1+ infiltrating macrophages inflammatory arthritis |
| | | synovium |
| RBX1 | 9978 | CCR2+ARG1+ infiltrating macrophages inflammatory arthritis |
| | | synovium |
| RGCC | 28984 | CCR2+ARG1+ infiltrating macrophages inflammatory arthritis |
| | | synovium |
| RNF149 | 284996 | CCR2+ARG1+ infiltrating macrophages inflammatory arthritis |
| | | synovium |
| RNF19B | 127544 | CCR2+ARG1+ infiltrating macrophages inflammatory arthritis |
| | | synovium |
| ROMO1 | 140823 | CCR2+ARG1+ infiltrating macrophages inflammatory arthritis |
| | | synovium |
| RPL12 | 6136 | CCR2+ARG1+ infiltrating macrophages inflammatory arthritis |
| | | synovium |
| RPL22 | 6146 | CCR2+ARG1+ infiltrating macrophages inflammatory arthritis |
| | | synovium |
| RPL22L1 | 200916 | CCR2+ARG1+ infiltrating macrophages inflammatory arthritis |
| | | synovium |
| RPL23 | 9349 | CCR2+ARG1+ infiltrating macrophages inflammatory arthritis |
| | | synovium |
| RPL27 | 6155 | CCR2+ARG1+ infiltrating macrophages inflammatory arthritis |
| | | synovium |
| RPL35 | 11224 | CCR2+ARG1+ infiltrating macrophages inflammatory arthritis |
| | | synovium |
| RPL36A | 6173 | CCR2+ARG1+ infiltrating macrophages inflammatory arthritis |
| | | synovium |
| RPL36AL | 6166 | CCR2+ARG1+ infiltrating macrophages inflammatory arthritis |
| | | synovium |
| RPL37A | 6168 | CCR2+ARG1+ infiltrating macrophages inflammatory arthritis |
| | | synovium |
| RPL38 | 6169 | CCR2+ARG1+ infiltrating macrophages inflammatory arthritis |
| | | synovium |
| RPL39 | 6170 | CCR2+ARG1+ infiltrating macrophages inflammatory arthritis |
| | | synovium |
| RPL5 | 6125 | CCR2+ARG1+ infiltrating macrophages inflammatory arthritis |
| | | synovium |
| RPLP1 | 6176 | CCR2+ARG1+ infiltrating macrophages inflammatory arthritis |
| | | synovium |
| RPLP2 | 6181 | CCR2+ARG1+ infiltrating macrophages inflammatory arthritis |
| | | synovium |
| RPS10 | 6204 | CCR2+ARG1+ infiltrating macrophages inflammatory arthritis |
| | | synovium |
| RPS12 | 6206 | CCR2+ARG1+ infiltrating macrophages inflammatory arthritis |
| | | synovium |
| RPS15A | 6210 | CCR2+ARG1+ infiltrating macrophages inflammatory arthritis |
| | | synovium |
| RPS17 | 6218 | CCR2+ARG1+ infiltrating macrophages inflammatory arthritis |
| | | synovium |
| RPS18 | 6222 | CCR2+ARG1+ infiltrating macrophages inflammatory arthritis |
| | | synovium |
| RPS19 | 6223 | CCR2+ARG1+ infiltrating macrophages inflammatory arthritis |
| | | synovium |
| RPS2 | 6187 | CCR2+ARG1+ infiltrating macrophages inflammatory arthritis |
| | | synovium |
| RPS21 | 6227 | CCR2+ARG1+ infiltrating macrophages inflammatory arthritis |
| | | synovium |
| RPS26 | 6231 | CCR2+ARG1+ infiltrating macrophages inflammatory arthritis |
| | | synovium |
| RPS27A | 6233 | CCR2+ARG1+ infiltrating macrophages inflammatory arthritis |
| | | synovium |
| RPS27L | 51065 | CCR2+ARG1+ infiltrating macrophages inflammatory arthritis |
| | | synovium |
| RPS3 | 6188 | CCR2+ARG1+ infiltrating macrophages inflammatory arthritis |
| | | synovium |
| RPS3A | 6189 | CCR2+ARG1+ infiltrating macrophages inflammatory arthritis |
| | | synovium |
| RPS8 | 6202 | CCR2+ARG1+ infiltrating macrophages inflammatory arthritis |
| | | synovium |
| S100A10 | 6281 | CCR2+ARG1+ infiltrating macrophages inflammatory arthritis |
| | | synovium |
| S100A11 | 6282 | CCR2+ARG1+ infiltrating macrophages inflammatory arthritis |
| | | synovium |
| S100A4 | 6275 | CCR2+ARG1+ infiltrating macrophages inflammatory arthritis |
| | | synovium |
| SDC1 | 6382 | CCR2+ARG1+ infiltrating macrophages inflammatory arthritis |
| | | synovium |
| SDC4 | 6385 | CCR2+ARG1+ infiltrating macrophages inflammatory arthritis |
| | | synovium |
| SEC61B | 10952 | CCR2+ARG1+ infiltrating macrophages inflammatory arthritis |
| | | synovium |
| SEMA4D | 10507 | CCR2+ARG1+ infiltrating macrophages inflammatory arthritis |
| | | synovium |
| SERBP1 | 26135 | CCR2+ARG1+ infiltrating macrophages inflammatory arthritis |
| | | synovium |
| SET | 6418 | CCR2+ARG1+ infiltrating macrophages inflammatory arthritis |
| | | synovium |
| SFPQ | 6421 | CCR2+ARG1+ infiltrating macrophages inflammatory arthritis |
| | | synovium |
| SGK1 | 6446 | CCR2+ARG1+ infiltrating macrophages inflammatory arthritis |
| | | synovium |
| SLC37A2 | 219855 | CCR2+ARG1+ infiltrating macrophages inflammatory arthritis |
| | | synovium |
| SLC43A2 | 124935 | CCR2+ARG1+ infiltrating macrophages inflammatory arthritis |
| | | synovium |
| SLIRP | 81892 | CCR2+ARG1+ infiltrating macrophages inflammatory arthritis |
| | | synovium |
| SMS | 6611 | CCR2+ARG1+ infiltrating macrophages inflammatory arthritis |
| | | synovium |
| SNHG6 | 641638 | CCR2+ARG1+ infiltrating macrophages inflammatory arthritis |
| | | synovium |
| SNRPD1 | 6632 | CCR2+ARG1+ infiltrating macrophages inflammatory arthritis |
| | | synovium |
| SNRPD2 | 6633 | CCR2+ARG1+ infiltrating macrophages inflammatory arthritis |
| | | synovium |
| SNRPD3 | 6634 | CCR2+ARG1+ infiltrating macrophages inflammatory arthritis |
| | | synovium |
| SNRPE | 6635 | CCR2+ARG1+ infiltrating macrophages inflammatory arthritis |
| | | synovium |
| SNRPF | 6636 | CCR2+ARG1+ infiltrating macrophages inflammatory arthritis |
| | | synovium |
| SNU13 | 4809 | CCR2+ARG1+ infiltrating macrophages inflammatory arthritis |
| | | synovium |
| SOD1 | 6647 | CCR2+ARG1+ infiltrating macrophages inflammatory arthritis |
| | | synovium |
| SOD2 | 6648 | CCR2+ARG1+ infiltrating macrophages inflammatory arthritis |
| | | synovium |
| SPP1 | 6696 | CCR2+ARG1+ infiltrating macrophages inflammatory arthritis |
| | | synovium |
| SQSTM1 | 8878 | CCR2+ARG1+ infiltrating macrophages inflammatory arthritis |
| | | synovium |
| SRSF3 | 6428 | CCR2+ARG1+ infiltrating macrophages inflammatory arthritis |
| | | synovium |
| SRSF7 | 6432 | CCR2+ARG1+ infiltrating macrophages inflammatory arthritis |
| | | synovium |
| TAF1D | 79101 | CCR2+ARG1+ infiltrating macrophages inflammatory arthritis |
| | | synovium |
| TCIRG1 | 10312 | CCR2+ARG1+ infiltrating macrophages inflammatory arthritis |
| | | synovium |
| TCP1 | 6950 | CCR2+ARG1+ infiltrating macrophages inflammatory arthritis |
| | | synovium |
| TCTEX1D2 | 255758 | CCR2+ARG1+ infiltrating macrophages inflammatory arthritis |
| | | synovium |
| TIMM10 | 26519 | CCR2+ARG1+ infiltrating macrophages inflammatory arthritis |
| | | synovium |
| TIMM13 | 26517 | CCR2+ARG1+ infiltrating macrophages inflammatory arthritis |
| | | synovium |
| TIMM17A | 10440 | CCR2+ARG1+ infiltrating macrophages inflammatory arthritis |
| | | synovium |
| TIMM8A | 1678 | CCR2+ARG1+ infiltrating macrophages inflammatory arthritis |
| | | synovium |
| TIMP1 | 7076 | CCR2+ARG1+ infiltrating macrophages inflammatory arthritis |
| | | synovium |
| TNFAIP2 | 7127 | CCR2+ARG1+ infiltrating macrophages inflammatory arthritis |
| | | synovium |
| TNFAIP3 | 7128 | CCR2+ARG1+ infiltrating macrophages inflammatory arthritis |
| | | synovium |
| TOMM20 | 9804 | CCR2+ARG1+ infiltrating macrophages inflammatory arthritis |
| | | synovium |
| TOMM40 | 10452 | CCR2+ARG1+ infiltrating macrophages inflammatory arthritis |
| | | synovium |
| TOMM5 | 401505 | CCR2+ARG1+ infiltrating macrophages inflammatory arthritis |
| | | synovium |
| TPI1 | 7167 | CCR2+ARG1+ infiltrating macrophages inflammatory arthritis |
| | | synovium |
| TPM1 | 7168 | CCR2+ARG1+ infiltrating macrophages inflammatory arthritis |
| | | synovium |
| TPT1 | 7178 | CCR2+ARG1+ infiltrating macrophages inflammatory arthritis |
| | | synovium |
| TRAF1 | 7185 | CCR2+ARG1+ infiltrating macrophages inflammatory arthritis |
| | | synovium |
| TSC22D1 | 8848 | CCR2+ARG1+ infiltrating macrophages inflammatory arthritis |
| | | synovium |
| TUBA1C | 84790 | CCR2+ARG1+ infiltrating macrophages inflammatory arthritis |
| | | synovium |
| TUBB4B | 10383 | CCR2+ARG1+ infiltrating macrophages inflammatory arthritis |
| | | synovium |
| TUBB6 | 84617 | CCR2+ARG1+ infiltrating macrophages inflammatory arthritis |
| | | synovium |
| TXN | 7295 | CCR2+ARG1+ infiltrating macrophages inflammatory arthritis |
| | | synovium |
| TXNRD1 | 7296 | CCR2+ARG1+ infiltrating macrophages inflammatory arthritis |
| | | synovium |
| U2AF1 | 7307 | CCR2+ARG1+ infiltrating macrophages inflammatory arthritis |
| | | synovium |
| UBE2A | 7319 | CCR2+ARG1+ infiltrating macrophages inflammatory arthritis |
| | | synovium |
| UBE2L3 | 7332 | CCR2+ARG1+ infiltrating macrophages inflammatory arthritis |
| | | synovium |
| UFM1 | 51569 | CCR2+ARG1+ infiltrating macrophages inflammatory arthritis |
| | | synovium |
| UQCR10 | 29796 | CCR2+ARG1+ infiltrating macrophages inflammatory arthritis |
| | | synovium |
| UQCR11 | 10975 | CCR2+ARG1+ infiltrating macrophages inflammatory arthritis |
| | | synovium |
| UQCRB | 7381 | CCR2+ARG1+ infiltrating macrophages inflammatory arthritis |
| | | synovium |
| UQCRQ | 27089 | CCR2+ARG1+ infiltrating macrophages inflammatory arthritis |
| | | synovium |
| USMG5 | 84833 | CCR2+ARG1+ infiltrating macrophages inflammatory arthritis |
| | | synovium |
| USP50 | 373509 | CCR2+ARG1+ infiltrating macrophages inflammatory arthritis |
| | | synovium |
| VIM | 7431 | CCR2+ARG1+ infiltrating macrophages inflammatory arthritis |
| | | synovium |
| YBX1 | 4904 | CCR2+ARG1+ infiltrating macrophages inflammatory arthritis |
| | | synovium |
| YBX3 | 8531 | CCR2+ARG1+ infiltrating macrophages inflammatory arthritis |
| | | synovium |
| YWHAG | 7532 | CCR2+ARG1+ infiltrating macrophages inflammatory arthritis |
| | | synovium |
| YWHAZ | 7534 | CCR2+ARG1+ infiltrating macrophages inflammatory arthritis |
| | | synovium |
| ZNF593 | 51042 | CCR2+ARG1+ infiltrating macrophages inflammatory arthritis |
| | | synovium |
| ACOT7 | 11332 | MHCII high dendritic cells inflammatory arthritis synovium |
| ACTG1 | 71 | MHCII high dendritic cells inflammatory arthritis synovium |
| ASS1 | 445 | MHCII high dendritic cells inflammatory arthritis synovium |
| ATOX1 | 475 | MHCII high dendritic cells inflammatory arthritis synovium |
| ATP5F1 | 515 | MHCII high dendritic cells inflammatory arthritis synovium |
| AVPI1 | 60370 | MHCII high dendritic cells inflammatory arthritis synovium |
| B4GALNT1 | 2583 | MHCII high dendritic cells inflammatory arthritis synovium |
| BATF3 | 55509 | MHCII high dendritic cells inflammatory arthritis synovium |
| BCL2A1 | 597 | MHCII high dendritic cells inflammatory arthritis synovium |
| BHLHE40 | 8553 | MHCII high dendritic cells inflammatory arthritis synovium |
| BLOC1S2 | 282991 | MHCII high dendritic cells inflammatory arthritis synovium |
| BRI3BP | 140707 | MHCII high dendritic cells inflammatory arthritis synovium |
| BTG2 | 7832 | MHCII high dendritic cells inflammatory arthritis synovium |
| C15orf48 | 84419 | MHCII high dendritic cells inflammatory arthritis synovium |
| C1orf21 | 81563 | MHCII high dendritic cells inflammatory arthritis synovium |
| C1orf54 | 79630 | MHCII high dendritic cells inflammatory arthritis synovium |
| CASP6 | 839 | MHCII high dendritic cells inflammatory arthritis synovium |
| CBFA2T3 | 863 | MHCII high dendritic cells inflammatory arthritis synovium |
| CCDC88A | 55704 | MHCII high dendritic cells inflammatory arthritis synovium |
| CCND1 | 595 | MHCII high dendritic cells inflammatory arthritis synovium |
| CCND3 | 896 | MHCII high dendritic cells inflammatory arthritis synovium |
| CCR2 | 729230 | MHCII high dendritic cells inflammatory arthritis synovium |
| CD24 | 10013941 | MHCII high dendritic cells inflammatory arthritis synovium |
| CD47 | 961 | MHCII high dendritic cells inflammatory arthritis synovium |
| CD52 | 1043 | MHCII high dendritic cells inflammatory arthritis synovium |
| CD72 | 971 | MHCII high dendritic cells inflammatory arthritis synovium |
| CD74 | 972 | MHCII high dendritic cells inflammatory arthritis synovium |
| CD86 | 942 | MHCII high dendritic cells inflammatory arthritis synovium |
| CDKN2D | 1032 | MHCII high dendritic cells inflammatory arthritis synovium |
| CFP | 5199 | MHCII high dendritic cells inflammatory arthritis synovium |
| CKB | 1152 | MHCII high dendritic cells inflammatory arthritis synovium |
| CLEC10A | 10462 | MHCII high dendritic cells inflammatory arthritis synovium |
| CLEC6A | 93978 | MHCII high dendritic cells inflammatory arthritis synovium |
| CNN2 | 1265 | MHCII high dendritic cells inflammatory arthritis synovium |
| CORO1A | 11151 | MHCII high dendritic cells inflammatory arthritis synovium |
| CSF2RB | 1439 | MHCII high dendritic cells inflammatory arthritis synovium |
| CSRNP1 | 64651 | MHCII high dendritic cells inflammatory arthritis synovium |
| CTSH | 1512 | MHCII high dendritic cells inflammatory arthritis synovium |
| CXCL16 | 58191 | MHCII high dendritic cells inflammatory arthritis synovium |
| CYTIP | 9595 | MHCII high dendritic cells inflammatory arthritis synovium |
| DAPP1 | 27071 | MHCII high dendritic cells inflammatory arthritis synovium |
| DBNL | 28988 | MHCII high dendritic cells inflammatory arthritis synovium |
| DDOST | 1650 | MHCII high dendritic cells inflammatory arthritis synovium |
| DENND4A | 10260 | MHCII high dendritic cells inflammatory arthritis synovium |
| DPYSL2 | 1808 | MHCII high dendritic cells inflammatory arthritis synovium |
| EEF1B2 | 1933 | MHCII high dendritic cells inflammatory arthritis synovium |
| EEF1G | 1937 | MHCII high dendritic cells inflammatory arthritis synovium |
| EIF3F | 8665 | MHCII high dendritic cells inflammatory arthritis synovium |
| EMB | 133418 | MHCII high dendritic cells inflammatory arthritis synovium |
| ETV3 | 2117 | MHCII high dendritic cells inflammatory arthritis synovium |
| EVA1B | 55194 | MHCII high dendritic cells inflammatory arthritis synovium |
| FABP5 | 2171 | MHCII high dendritic cells inflammatory arthritis synovium |
| FAM129A | 116496 | MHCII high dendritic cells inflammatory arthritis synovium |
| FBL | 2091 | MHCII high dendritic cells inflammatory arthritis synovium |
| FDPS | 2224 | MHCII high dendritic cells inflammatory arthritis synovium |
| FGL2 | 10875 | MHCII high dendritic cells inflammatory arthritis synovium |
| FGR | 2268 | MHCII high dendritic cells inflammatory arthritis synovium |
| FH | 2271 | MHCII high dendritic cells inflammatory arthritis synovium |
| GLIPR2 | 152007 | MHCII high dendritic cells inflammatory arthritis synovium |
| GM2A | 2760 | MHCII high dendritic cells inflammatory arthritis synovium |
| GPR132 | 29933 | MHCII high dendritic cells inflammatory arthritis synovium |
| GPR171 | 29909 | MHCII high dendritic cells inflammatory arthritis synovium |
| GRK3 | 157 | MHCII high dendritic cells inflammatory arthritis synovium |
| H2AFY | 9555 | MHCII high dendritic cells inflammatory arthritis synovium |
| H2AFZ | 3015 | MHCII high dendritic cells inflammatory arthritis synovium |
| HLA-DMA | 3108 | MHCII high dendritic cells inflammatory arthritis synovium |
| HLA-DMB | 3109 | MHCII high dendritic cells inflammatory arthritis synovium |
| HLA-DQA1 | 3117 | MHCII high dendritic cells inflammatory arthritis synovium |
| HLA-DQB1 | 3119 | MHCII high dendritic cells inflammatory arthritis synovium |
| HLA-DRB5 | 3127 | MHCII high dendritic cells inflammatory arthritis synovium |
| HM13 | 81502 | MHCII high dendritic cells inflammatory arthritis synovium |
| HSD17B10 | 3028 | MHCII high dendritic cells inflammatory arthritis synovium |
| HSPA1A | 3303 | MHCII high dendritic cells inflammatory arthritis synovium |
| ID2 | 3398 | MHCII high dendritic cells inflammatory arthritis synovium |
| IFITM2 | 10581 | MHCII high dendritic cells inflammatory arthritis synovium |
| IFNGR1 | 3459 | MHCII high dendritic cells inflammatory arthritis synovium |
| IL1B | 3553 | MHCII high dendritic cells inflammatory arthritis synovium |
| IL1R2 | 7850 | MHCII high dendritic cells inflammatory arthritis synovium |
| IL2RG | 3561 | MHCII high dendritic cells inflammatory arthritis synovium |
| ITGAX | 3687 | MHCII high dendritic cells inflammatory arthritis synovium |
| ITGB7 | 3695 | MHCII high dendritic cells inflammatory arthritis synovium |
| JAK2 | 3717 | MHCII high dendritic cells inflammatory arthritis synovium |
| JAML | 120425 | MHCII high dendritic cells inflammatory arthritis synovium |
| KIAA0040 | 9674 | MHCII high dendritic cells inflammatory arthritis synovium |
| KLRB1 | 3820 | MHCII high dendritic cells inflammatory arthritis synovium |
| KLRD1 | 3824 | MHCII high dendritic cells inflammatory arthritis synovium |
| LACTB | 114294 | MHCII high dendritic cells inflammatory arthritis synovium |
| LIMD2 | 80774 | MHCII high dendritic cells inflammatory arthritis synovium |
| LMNB1 | 4001 | MHCII high dendritic cells inflammatory arthritis synovium |
| LMO4 | 8543 | MHCII high dendritic cells inflammatory arthritis synovium |
| LSP1 | 4046 | MHCII high dendritic cells inflammatory arthritis synovium |
| LSR | 51599 | MHCII high dendritic cells inflammatory arthritis synovium |
| LTB4R | 1241 | MHCII high dendritic cells inflammatory arthritis synovium |
| MARCKSL1 | 65108 | MHCII high dendritic cells inflammatory arthritis synovium |
| MED30 | 90390 | MHCII high dendritic cells inflammatory arthritis synovium |
| MPC1 | 51660 | MHCII high dendritic cells inflammatory arthritis synovium |
| MYD88 | 4615 | MHCII high dendritic cells inflammatory arthritis synovium |
| MYO1G | 64005 | MHCII high dendritic cells inflammatory arthritis synovium |
| NAAA | 27163 | MHCII high dendritic cells inflammatory arthritis synovium |
| NAGA | 4668 | MHCII high dendritic cells inflammatory arthritis synovium |
| NAPSA | 9476 | MHCII high dendritic cells inflammatory arthritis synovium |
| NDUFA6 | 4700 | MHCII high dendritic cells inflammatory arthritis synovium |
| NR4A2 | 4929 | MHCII high dendritic cells inflammatory arthritis synovium |
| OLFM1 | 10439 | MHCII high dendritic cells inflammatory arthritis synovium |
| PAK1 | 5058 | MHCII high dendritic cells inflammatory arthritis synovium |
| PDLIM1 | 9124 | MHCII high dendritic cells inflammatory arthritis synovium |
| PFKP | 5214 | MHCII high dendritic cells inflammatory arthritis synovium |
| PIM1 | 5292 | MHCII high dendritic cells inflammatory arthritis synovium |
| PKIB | 5570 | MHCII high dendritic cells inflammatory arthritis synovium |
| PLBD1 | 79887 | MHCII high dendritic cells inflammatory arthritis synovium |
| PLSCR1 | 5359 | MHCII high dendritic cells inflammatory arthritis synovium |
| PMVK | 10654 | MHCII high dendritic cells inflammatory arthritis synovium |
| PRCP | 5547 | MHCII high dendritic cells inflammatory arthritis synovium |
| PRKAR2A | 5576 | MHCII high dendritic cells inflammatory arthritis synovium |
| PSMB8 | 5696 | MHCII high dendritic cells inflammatory arthritis synovium |
| PSMB9 | 5698 | MHCII high dendritic cells inflammatory arthritis synovium |
| PSME1 | 5720 | MHCII high dendritic cells inflammatory arthritis synovium |
| PTPRC | 5788 | MHCII high dendritic cells inflammatory arthritis synovium |
| RAMP1 | 10267 | MHCII high dendritic cells inflammatory arthritis synovium |
| RARA | 5914 | MHCII high dendritic cells inflammatory arthritis synovium |
| REL | 5966 | MHCII high dendritic cells inflammatory arthritis synovium |
| RGS1 | 5996 | MHCII high dendritic cells inflammatory arthritis synovium |
| RIN3 | 79890 | MHCII high dendritic cells inflammatory arthritis synovium |
| ROGDI | 79641 | MHCII high dendritic cells inflammatory arthritis synovium |
| RPL13 | 6137 | MHCII high dendritic cells inflammatory arthritis synovium |
| RPL14 | 9045 | MHCII high dendritic cells inflammatory arthritis synovium |
| RPL15 | 6138 | MHCII high dendritic cells inflammatory arthritis synovium |
| RPL17 | 6139 | MHCII high dendritic cells inflammatory arthritis synovium |
| RPL32 | 6161 | MHCII high dendritic cells inflammatory arthritis synovium |
| RPL39 | 6170 | MHCII high dendritic cells inflammatory arthritis synovium |
| RPL4 | 6124 | MHCII high dendritic cells inflammatory arthritis synovium |
| RPLP0 | 6175 | MHCII high dendritic cells inflammatory arthritis synovium |
| RPS11 | 6205 | MHCII high dendritic cells inflammatory arthritis synovium |
| RPS16 | 6217 | MHCII high dendritic cells inflammatory arthritis synovium |
| RPS18 | 6222 | MHCII high dendritic cells inflammatory arthritis synovium |
| RPS19 | 6223 | MHCII high dendritic cells inflammatory arthritis synovium |
| RPS4Y1 | 6192 | MHCII high dendritic cells inflammatory arthritis synovium |
| RPS6 | 6194 | MHCII high dendritic cells inflammatory arthritis synovium |
| RPS7 | 6201 | MHCII high dendritic cells inflammatory arthritis synovium |
| RPSA | 3921 | MHCII high dendritic cells inflammatory arthritis synovium |
| S100A11 | 6282 | MHCII high dendritic cells inflammatory arthritis synovium |
| SCAND1 | 51282 | MHCII high dendritic cells inflammatory arthritis synovium |
| SDF2L1 | 23753 | MHCII high dendritic cells inflammatory arthritis synovium |
| SEC61B | 10952 | MHCII high dendritic cells inflammatory arthritis synovium |
| SELPLG | 6404 | MHCII high dendritic cells inflammatory arthritis synovium |
| SLAMF9 | 89886 | MHCII high dendritic cells inflammatory arthritis synovium |
| SPINT1 | 6692 | MHCII high dendritic cells inflammatory arthritis synovium |
| SRI | 6717 | MHCII high dendritic cells inflammatory arthritis synovium |
| ST3GAL4 | 6484 | MHCII high dendritic cells inflammatory arthritis synovium |
| SUB1 | 10923 | MHCII high dendritic cells inflammatory arthritis synovium |
| SYNGR2 | 9144 | MHCII high dendritic cells inflammatory arthritis synovium |
| TAP1 | 6890 | MHCII high dendritic cells inflammatory arthritis synovium |
| TAX1BP3 | 30851 | MHCII high dendritic cells inflammatory arthritis synovium |
| TES | 26136 | MHCII high dendritic cells inflammatory arthritis synovium |
| TIMP1 | 7076 | MHCII high dendritic cells inflammatory arthritis synovium |
| TMEM123 | 114908 | MHCII high dendritic cells inflammatory arthritis synovium |
| TMEM173 | 340061 | MHCII high dendritic cells inflammatory arthritis synovium |
| TMEM176A | 55365 | MHCII high dendritic cells inflammatory arthritis synovium |
| TMEM176B | 28959 | MHCII high dendritic cells inflammatory arthritis synovium |
| TMSB10 | 9168 | MHCII high dendritic cells inflammatory arthritis synovium |
| TNIP3 | 79931 | MHCII high dendritic cells inflammatory arthritis synovium |
| TRAF1 | 7185 | MHCII high dendritic cells inflammatory arthritis synovium |
| TRAPPC5 | 126003 | MHCII high dendritic cells inflammatory arthritis synovium |
| TSPAN13 | 27075 | MHCII high dendritic cells inflammatory arthritis synovium |
| TUBA1A | 7846 | MHCII high dendritic cells inflammatory arthritis synovium |
| UNC119 | 9094 | MHCII high dendritic cells inflammatory arthritis synovium |
| UVRAG | 7405 | MHCII high dendritic cells inflammatory arthritis synovium |
| VASP | 7408 | MHCII high dendritic cells inflammatory arthritis synovium |
| VRK1 | 7443 | MHCII high dendritic cells inflammatory arthritis synovium |
| YWHAH | 7533 | MHCII high dendritic cells inflammatory arthritis synovium |
| ZYX | 7791 | MHCII high dendritic cells inflammatory arthritis synovium |
| ACOT7 | 11332 | STMN1+ proliferating cells inflammatory arthritis synovium |
| ACP5 | 54 | STMN1+ proliferating cells inflammatory arthritis synovium |
| ACTL6A | 86 | STMN1+ proliferating cells inflammatory arthritis synovium |
| ACTN4 | 81 | STMN1+ proliferating cells inflammatory arthritis synovium |
| ADSS | 159 | STMN1+ proliferating cells inflammatory arthritis synovium |
| AGPAT5 | 55326 | STMN1+ proliferating cells inflammatory arthritis synovium |
| AIMP2 | 7965 | STMN1+ proliferating cells inflammatory arthritis synovium |
| AK2 | 204 | STMN1+ proliferating cells inflammatory arthritis synovium |
| AK6 | 1.02E+08 | STMN1+ proliferating cells inflammatory arthritis synovium |
| ALYREF | 10189 | STMN1+ proliferating cells inflammatory arthritis synovium |
| ANAPC11 | 51529 | STMN1+ proliferating cells inflammatory arthritis synovium |
| ANAPC5 | 51433 | STMN1+ proliferating cells inflammatory arthritis synovium |
| ANLN | 54443 | STMN1+ proliferating cells inflammatory arthritis synovium |
| ANP32B | 10541 | STMN1+ proliferating cells inflammatory arthritis synovium |
| ANP32E | 81611 | STMN1+ proliferating cells inflammatory arthritis synovium |
| AP1S1 | 1174 | STMN1+ proliferating cells inflammatory arthritis synovium |
| APEX1 | 328 | STMN1+ proliferating cells inflammatory arthritis synovium |
| ARHGAP11A | 9824 | STMN1+ proliferating cells inflammatory arthritis synovium |
| ARID1A | 8289 | STMN1+ proliferating cells inflammatory arthritis synovium |
| ARL6IP1 | 23204 | STMN1+ proliferating cells inflammatory arthritis synovium |
| ARL8B | 55207 | STMN1+ proliferating cells inflammatory arthritis synovium |
| ARPP19 | 10776 | STMN1+ proliferating cells inflammatory arthritis synovium |
| ASF1B | 55723 | STMN1+ proliferating cells inflammatory arthritis synovium |
| ASPM | 259266 | STMN1+ proliferating cells inflammatory arthritis synovium |
| ATAD2 | 29028 | STMN1+ proliferating cells inflammatory arthritis synovium |
| ATP5B | 506 | STMN1+ proliferating cells inflammatory arthritis synovium |
| ATP5F1 | 515 | STMN1+ proliferating cells inflammatory arthritis synovium |
| ATP5G1 | 516 | STMN1+ proliferating cells inflammatory arthritis synovium |
| ATP5G2 | 517 | STMN1+ proliferating cells inflammatory arthritis synovium |
| ATP5G3 | 518 | STMN1+ proliferating cells inflammatory arthritis synovium |
| ATP5J2 | 9551 | STMN1+ proliferating cells inflammatory arthritis synovium |
| ATP5L | 10632 | STMN1+ proliferating cells inflammatory arthritis synovium |
| ATP5ME | 521 | STMN1+ proliferating cells inflammatory arthritis synovium |
| ATP5O | 539 | STMN1+ proliferating cells inflammatory arthritis synovium |
| ATPIF1 | 93974 | STMN1+ proliferating cells inflammatory arthritis synovium |
| AURKA | 6790 | STMN1+ proliferating cells inflammatory arthritis synovium |
| AURKB | 9212 | STMN1+ proliferating cells inflammatory arthritis synovium |
| BAG1 | 573 | STMN1+ proliferating cells inflammatory arthritis synovium |
| BANF1 | 8815 | STMN1+ proliferating cells inflammatory arthritis synovium |
| BAZ1A | 11177 | STMN1+ proliferating cells inflammatory arthritis synovium |
| BCKDK | 10295 | STMN1+ proliferating cells inflammatory arthritis synovium |
| BDH2 | 56898 | STMN1+ proliferating cells inflammatory arthritis synovium |
| BEX3 | 27018 | STMN1+ proliferating cells inflammatory arthritis synovium |
| BICD2 | 23299 | STMN1+ proliferating cells inflammatory arthritis synovium |
| BIRC2 | 329 | STMN1+ proliferating cells inflammatory arthritis synovium |
| BIRC5 | 332 | STMN1+ proliferating cells inflammatory arthritis synovium |
| BOLA2 | 552900 | STMN1+ proliferating cells inflammatory arthritis synovium |
| BOLA3 | 388962 | STMN1+ proliferating cells inflammatory arthritis synovium |
| BRD7 | 29117 | STMN1+ proliferating cells inflammatory arthritis synovium |
| BRIX1 | 55299 | STMN1+ proliferating cells inflammatory arthritis synovium |
| BUB1 | 699 | STMN1+ proliferating cells inflammatory arthritis synovium |
| BUB1B | 701 | STMN1+ proliferating cells inflammatory arthritis synovium |
| BUB3 | 9184 | STMN1+ proliferating cells inflammatory arthritis synovium |
| C11orf58 | 10944 | STMN1+ proliferating cells inflammatory arthritis synovium |
| C14orf2 | 9556 | STMN1+ proliferating cells inflammatory arthritis synovium |
| C1QBP | 708 | STMN1+ proliferating cells inflammatory arthritis synovium |
| C20orf24 | 55969 | STMN1+ proliferating cells inflammatory arthritis synovium |
| C4ORF3 | 401152 | STMN1+ proliferating cells inflammatory arthritis synovium |
| C6orf62 | 81688 | STMN1+ proliferating cells inflammatory arthritis synovium |
| CACYBP | 27101 | STMN1+ proliferating cells inflammatory arthritis synovium |
| CALM3 | 808 | STMN1+ proliferating cells inflammatory arthritis synovium |
| CAPRIN1 | 4076 | STMN1+ proliferating cells inflammatory arthritis synovium |
| CAV1 | 857 | STMN1+ proliferating cells inflammatory arthritis synovium |
| CAV2 | 858 | STMN1+ proliferating cells inflammatory arthritis synovium |
| CBFB | 865 | STMN1+ proliferating cells inflammatory arthritis synovium |
| CBX1 | 10951 | STMN1+ proliferating cells inflammatory arthritis synovium |
| CBX3 | 11335 | STMN1+ proliferating cells inflammatory arthritis synovium |
| CBX5 | 23468 | STMN1+ proliferating cells inflammatory arthritis synovium |
| CCDC25 | 55246 | STMN1+ proliferating cells inflammatory arthritis synovium |
| CCDC34 | 91057 | STMN1+ proliferating cells inflammatory arthritis synovium |
| CCDC88A | 55704 | STMN1+ proliferating cells inflammatory arthritis synovium |
| CCNA2 | 890 | STMN1+ proliferating cells inflammatory arthritis synovium |
| CCNB1 | 891 | STMN1+ proliferating cells inflammatory arthritis synovium |
| CCNB2 | 9133 | STMN1+ proliferating cells inflammatory arthritis synovium |
| CCND1 | 595 | STMN1+ proliferating cells inflammatory arthritis synovium |
| CCNE2 | 9134 | STMN1+ proliferating cells inflammatory arthritis synovium |
| CCT2 | 10576 | STMN1+ proliferating cells inflammatory arthritis synovium |
| CCT3 | 7203 | STMN1+ proliferating cells inflammatory arthritis synovium |
| CCT7 | 10574 | STMN1+ proliferating cells inflammatory arthritis synovium |
| CCT8 | 10694 | STMN1+ proliferating cells inflammatory arthritis synovium |
| CD44 | 960 | STMN1+ proliferating cells inflammatory arthritis synovium |
| CDC20 | 991 | STMN1+ proliferating cells inflammatory arthritis synovium |
| CDC45 | 8318 | STMN1+ proliferating cells inflammatory arthritis synovium |
| CDCA2 | 157313 | STMN1+ proliferating cells inflammatory arthritis synovium |
| CDCA3 | 83461 | STMN1+ proliferating cells inflammatory arthritis synovium |
| CDCA4 | 55038 | STMN1+ proliferating cells inflammatory arthritis synovium |
| CDCA8 | 55143 | STMN1+ proliferating cells inflammatory arthritis synovium |
| CDK1 | 983 | STMN1+ proliferating cells inflammatory arthritis synovium |
| CDK4 | 1019 | STMN1+ proliferating cells inflammatory arthritis synovium |
| CDKN2C | 1031 | STMN1+ proliferating cells inflammatory arthritis synovium |
| CDKN2D | 1032 | STMN1+ proliferating cells inflammatory arthritis synovium |
| CDKN3 | 1033 | STMN1+ proliferating cells inflammatory arthritis synovium |
| CENPA | 1058 | STMN1+ proliferating cells inflammatory arthritis synovium |
| CENPE | 1062 | STMN1+ proliferating cells inflammatory arthritis synovium |
| CENPF | 1063 | STMN1+ proliferating cells inflammatory arthritis synovium |
| CENPH | 64946 | STMN1+ proliferating cells inflammatory arthritis synovium |
| CENPK | 64105 | STMN1+ proliferating cells inflammatory arthritis synovium |
| CENPM | 79019 | STMN1+ proliferating cells inflammatory arthritis synovium |
| CENPQ | 55166 | STMN1+ proliferating cells inflammatory arthritis synovium |
| CENPS | 370708 | STMN1+ proliferating cells inflammatory arthritis synovium |
| CENPW | 387103 | STMN1+ proliferating cells inflammatory arthritis synovium |
| CENPX | 201254 | STMN1+ proliferating cells inflammatory arthritis synovium |
| CEP55 | 55165 | STMN1+ proliferating cells inflammatory arthritis synovium |
| CEP83 | 51134 | STMN1+ proliferating cells inflammatory arthritis synovium |
| CHCHD1 | 118487 | STMN1+ proliferating cells inflammatory arthritis synovium |
| CHCHD10 | 400916 | STMN1+ proliferating cells inflammatory arthritis synovium |
| CISD1 | 55847 | STMN1+ proliferating cells inflammatory arthritis synovium |
| CITED2 | 10370 | STMN1+ proliferating cells inflammatory arthritis synovium |
| CKAP2 | 26586 | STMN1+ proliferating cells inflammatory arthritis synovium |
| CKAP2L | 150468 | STMN1+ proliferating cells inflammatory arthritis synovium |
| CKAP4 | 10970 | STMN1+ proliferating cells inflammatory arthritis synovium |
| CKAP5 | 9793 | STMN1+ proliferating cells inflammatory arthritis synovium |
| CKS1B | 1163 | STMN1+ proliferating cells inflammatory arthritis synovium |
| CKS2 | 1164 | STMN1+ proliferating cells inflammatory arthritis synovium |
| CLIC4 | 25932 | STMN1+ proliferating cells inflammatory arthritis synovium |
| CLSPN | 63967 | STMN1+ proliferating cells inflammatory arthritis synovium |
| CMC2 | 56942 | STMN1+ proliferating cells inflammatory arthritis synovium |
| CNIH4 | 29097 | STMN1+ proliferating cells inflammatory arthritis synovium |
| CNOT6 | 57472 | STMN1+ proliferating cells inflammatory arthritis synovium |
| COPS9 | 150678 | STMN1+ proliferating cells inflammatory arthritis synovium |
| COQ7 | 10229 | STMN1+ proliferating cells inflammatory arthritis synovium |
| COX20 | 116228 | STMN1+ proliferating cells inflammatory arthritis synovium |
| COX5A | 9377 | STMN1+ proliferating cells inflammatory arthritis synovium |
| COX5B | 1329 | STMN1+ proliferating cells inflammatory arthritis synovium |
| COX6C | 1345 | STMN1+ proliferating cells inflammatory arthritis synovium |
| COX7A2 | 1347 | STMN1+ proliferating cells inflammatory arthritis synovium |
| COX7C | 1350 | STMN1+ proliferating cells inflammatory arthritis synovium |
| CPSF2 | 53981 | STMN1+ proliferating cells inflammatory arthritis synovium |
| CRIP2 | 1397 | STMN1+ proliferating cells inflammatory arthritis synovium |
| CSRP1 | 1465 | STMN1+ proliferating cells inflammatory arthritis synovium |
| CTCF | 10664 | STMN1+ proliferating cells inflammatory arthritis synovium |
| CTNNB1 | 1499 | STMN1+ proliferating cells inflammatory arthritis synovium |
| CTSK | 1513 | STMN1+ proliferating cells inflammatory arthritis synovium |
| CYB5R3 | 1727 | STMN1+ proliferating cells inflammatory arthritis synovium |
| CYC1 | 1537 | STMN1+ proliferating cells inflammatory arthritis synovium |
| CYCS | 54205 | STMN1+ proliferating cells inflammatory arthritis synovium |
| DAZAP1 | 26528 | STMN1+ proliferating cells inflammatory arthritis synovium |
| DBF4 | 10926 | STMN1+ proliferating cells inflammatory arthritis synovium |
| DCK | 1633 | STMN1+ proliferating cells inflammatory arthritis synovium |
| DCTPP1 | 79077 | STMN1+ proliferating cells inflammatory arthritis synovium |
| DCUN1D5 | 84259 | STMN1+ proliferating cells inflammatory arthritis synovium |
| DDT | 1652 | STMN1+ proliferating cells inflammatory arthritis synovium |
| DDX18 | 8886 | STMN1+ proliferating cells inflammatory arthritis synovium |
| DDX21 | 9188 | STMN1+ proliferating cells inflammatory arthritis synovium |
| DDX39A | 10212 | STMN1+ proliferating cells inflammatory arthritis synovium |
| DDX39B | 7919 | STMN1+ proliferating cells inflammatory arthritis synovium |
| DEK | 7913 | STMN1+ proliferating cells inflammatory arthritis synovium |
| DENR | 8562 | STMN1+ proliferating cells inflammatory arthritis synovium |
| DHFR | 1719 | STMN1+ proliferating cells inflammatory arthritis synovium |
| DHX15 | 1665 | STMN1+ proliferating cells inflammatory arthritis synovium |
| DHX9 | 1660 | STMN1+ proliferating cells inflammatory arthritis synovium |
| DIAPH3 | 81624 | STMN1+ proliferating cells inflammatory arthritis synovium |
| DKC1 | 1736 | STMN1+ proliferating cells inflammatory arthritis synovium |
| DLGAP5 | 9787 | STMN1+ proliferating cells inflammatory arthritis synovium |
| DNAJC2 | 27000 | STMN1+ proliferating cells inflammatory arthritis synovium |
| DNAJC9 | 23234 | STMN1+ proliferating cells inflammatory arthritis synovium |
| DNMT1 | 1786 | STMN1+ proliferating cells inflammatory arthritis synovium |
| DNPH1 | 10591 | STMN1+ proliferating cells inflammatory arthritis synovium |
| DPY30 | 84661 | STMN1+ proliferating cells inflammatory arthritis synovium |
| DTYMK | 1841 | STMN1+ proliferating cells inflammatory arthritis synovium |
| DUT | 1854 | STMN1+ proliferating cells inflammatory arthritis synovium |
| DYNLL2 | 140735 | STMN1+ proliferating cells inflammatory arthritis synovium |
| DYNLT1 | 6993 | STMN1+ proliferating cells inflammatory arthritis synovium |
| EEF1B2 | 1933 | STMN1+ proliferating cells inflammatory arthritis synovium |
| EEF1D | 1936 | STMN1+ proliferating cells inflammatory arthritis synovium |
| EEF1E1 | 9521 | STMN1+ proliferating cells inflammatory arthritis synovium |
| EEF1G | 1937 | STMN1+ proliferating cells inflammatory arthritis synovium |
| EIF1AD | 84285 | STMN1+ proliferating cells inflammatory arthritis synovium |
| EIF1AY | 9086 | STMN1+ proliferating cells inflammatory arthritis synovium |
| EIF2S2 | 8894 | STMN1+ proliferating cells inflammatory arthritis synovium |
| EIF3A | 8661 | STMN1+ proliferating cells inflammatory arthritis synovium |
| EIF3C | 8663 | STMN1+ proliferating cells inflammatory arthritis synovium |
| EIF3J | 8669 | STMN1+ proliferating cells inflammatory arthritis synovium |
| EIF4G2 | 1982 | STMN1+ proliferating cells inflammatory arthritis synovium |
| EIF5A | 1984 | STMN1+ proliferating cells inflammatory arthritis synovium |
| EIF6 | 3692 | STMN1+ proliferating cells inflammatory arthritis synovium |
| ELAVL1 | 1994 | STMN1+ proliferating cells inflammatory arthritis synovium |
| ELOC | 6921 | STMN1+ proliferating cells inflammatory arthritis synovium |
| EMG1 | 10436 | STMN1+ proliferating cells inflammatory arthritis synovium |
| ENY2 | 56943 | STMN1+ proliferating cells inflammatory arthritis synovium |
| ERH | 2079 | STMN1+ proliferating cells inflammatory arthritis synovium |
| ERI1 | 90459 | STMN1+ proliferating cells inflammatory arthritis synovium |
| ESCO2 | 157570 | STMN1+ proliferating cells inflammatory arthritis synovium |
| ETFB | 2109 | STMN1+ proliferating cells inflammatory arthritis synovium |
| EXOSC8 | 11340 | STMN1+ proliferating cells inflammatory arthritis synovium |
| EZH2 | 2146 | STMN1+ proliferating cells inflammatory arthritis synovium |
| FABP5 | 2171 | STMN1+ proliferating cells inflammatory arthritis synovium |
| FAM111A | 63901 | STMN1+ proliferating cells inflammatory arthritis synovium |
| FAM133B | 257415 | STMN1+ proliferating cells inflammatory arthritis synovium |
| FBL | 2091 | STMN1+ proliferating cells inflammatory arthritis synovium |
| FBXO5 | 26271 | STMN1+ proliferating cells inflammatory arthritis synovium |
| FEN1 | 2237 | STMN1+ proliferating cells inflammatory arthritis synovium |
| FIGNL1 | 63979 | STMN1+ proliferating cells inflammatory arthritis synovium |
| FKBP2 | 2286 | STMN1+ proliferating cells inflammatory arthritis synovium |
| FKBP3 | 2287 | STMN1+ proliferating cells inflammatory arthritis synovium |
| FKBP4 | 2288 | STMN1+ proliferating cells inflammatory arthritis synovium |
| FUS | 2521 | STMN1+ proliferating cells inflammatory arthritis synovium |
| FXN | 2395 | STMN1+ proliferating cells inflammatory arthritis synovium |
| G3BP1 | 10146 | STMN1+ proliferating cells inflammatory arthritis synovium |
| GADD45GIP1 | 90480 | STMN1+ proliferating cells inflammatory arthritis synovium |
| GAR1 | 54433 | STMN1+ proliferating cells inflammatory arthritis synovium |
| GAS2L3 | 283431 | STMN1+ proliferating cells inflammatory arthritis synovium |
| GATM | 2628 | STMN1+ proliferating cells inflammatory arthritis synovium |
| GEMIN6 | 79833 | STMN1+ proliferating cells inflammatory arthritis synovium |
| GINS2 | 51659 | STMN1+ proliferating cells inflammatory arthritis synovium |
| GJA1 | 2697 | STMN1+ proliferating cells inflammatory arthritis synovium |
| GLIPR1 | 11010 | STMN1+ proliferating cells inflammatory arthritis synovium |
| GMNN | 51053 | STMN1+ proliferating cells inflammatory arthritis synovium |
| GNL3 | 26354 | STMN1+ proliferating cells inflammatory arthritis synovium |
| GSPT1 | 2935 | STMN1+ proliferating cells inflammatory arthritis synovium |
| GTF2H5 | 404672 | STMN1+ proliferating cells inflammatory arthritis synovium |
| H1-4 | 3008 | STMN1+ proliferating cells inflammatory arthritis synovium |
| H1-5 | 3009 | STMN1+ proliferating cells inflammatory arthritis synovium |
| H2AFV | 94239 | STMN1+ proliferating cells inflammatory arthritis synovium |
| H2AFX | 3014 | STMN1+ proliferating cells inflammatory arthritis synovium |
| H2AFY | 9555 | STMN1+ proliferating cells inflammatory arthritis synovium |
| H2AFZ | 3015 | STMN1+ proliferating cells inflammatory arthritis synovium |
| HACD1 | 9200 | STMN1+ proliferating cells inflammatory arthritis synovium |
| HAT1 | 8520 | STMN1+ proliferating cells inflammatory arthritis synovium |
| HAUS1 | 115106 | STMN1+ proliferating cells inflammatory arthritis synovium |
| HAUS4 | 54930 | STMN1+ proliferating cells inflammatory arthritis synovium |
| HDAC2 | 3066 | STMN1+ proliferating cells inflammatory arthritis synovium |
| HDGF | 3068 | STMN1+ proliferating cells inflammatory arthritis synovium |
| HELLS | 3070 | STMN1+ proliferating cells inflammatory arthritis synovium |
| HINT1 | 3094 | STMN1+ proliferating cells inflammatory arthritis synovium |
| HIRIP3 | 8479 | STMN1+ proliferating cells inflammatory arthritis synovium |
| HIST1H1D | 3007 | STMN1+ proliferating cells inflammatory arthritis synovium |
| HIST1H2AL | 8332 | STMN1+ proliferating cells inflammatory arthritis synovium |
| HIST1H4A | 8359 | STMN1+ proliferating cells inflammatory arthritis synovium |
| HIST2H2AC | 8338 | STMN1+ proliferating cells inflammatory arthritis synovium |
| HJURP | 55355 | STMN1+ proliferating cells inflammatory arthritis synovium |
| HMGB1 | 3146 | STMN1+ proliferating cells inflammatory arthritis synovium |
| HMGB2 | 3148 | STMN1+ proliferating cells inflammatory arthritis synovium |
| HMGB3 | 3149 | STMN1+ proliferating cells inflammatory arthritis synovium |
| HMGN1 | 3150 | STMN1+ proliferating cells inflammatory arthritis synovium |
| HMGN2 | 3151 | STMN1+ proliferating cells inflammatory arthritis synovium |
| HMGN5 | 79366 | STMN1+ proliferating cells inflammatory arthritis synovium |
| HMMR | 3161 | STMN1+ proliferating cells inflammatory arthritis synovium |
| HNRNPA0 | 10949 | STMN1+ proliferating cells inflammatory arthritis synovium |
| HNRNPA1 | 3178 | STMN1+ proliferating cells inflammatory arthritis synovium |
| HNRNPA2B1 | 3181 | STMN1+ proliferating cells inflammatory arthritis synovium |
| HNRNPA3 | 220988 | STMN1+ proliferating cells inflammatory arthritis synovium |
| HNRNPAB | 3182 | STMN1+ proliferating cells inflammatory arthritis synovium |
| HNRNPD | 3184 | STMN1+ proliferating cells inflammatory arthritis synovium |
| HNRNPM | 4670 | STMN1+ proliferating cells inflammatory arthritis synovium |
| HNRNPU | 3192 | STMN1+ proliferating cells inflammatory arthritis synovium |
| HP1BP3 | 50809 | STMN1+ proliferating cells inflammatory arthritis synovium |
| HPF1 | 54969 | STMN1+ proliferating cells inflammatory arthritis synovium |
| HSP90AA1 | 3320 | STMN1+ proliferating cells inflammatory arthritis synovium |
| HSP90AB1 | 3326 | STMN1+ proliferating cells inflammatory arthritis synovium |
| HSP90B1 | 7184 | STMN1+ proliferating cells inflammatory arthritis synovium |
| HSPA14 | 51182 | STMN1+ proliferating cells inflammatory arthritis synovium |
| HSPA9 | 3313 | STMN1+ proliferating cells inflammatory arthritis synovium |
| HSPD1 | 3329 | STMN1+ proliferating cells inflammatory arthritis synovium |
| HSPE1 | 3336 | STMN1+ proliferating cells inflammatory arthritis synovium |
| HUWE1 | 10075 | STMN1+ proliferating cells inflammatory arthritis synovium |
| IDH3A | 3419 | STMN1+ proliferating cells inflammatory arthritis synovium |
| ILF2 | 3608 | STMN1+ proliferating cells inflammatory arthritis synovium |
| ILF3 | 3609 | STMN1+ proliferating cells inflammatory arthritis synovium |
| IMMT | 10989 | STMN1+ proliferating cells inflammatory arthritis synovium |
| IMPDH2 | 3615 | STMN1+ proliferating cells inflammatory arthritis synovium |
| INCENP | 3619 | STMN1+ proliferating cells inflammatory arthritis synovium |
| ING1 | 3621 | STMN1+ proliferating cells inflammatory arthritis synovium |
| IPO5 | 3843 | STMN1+ proliferating cells inflammatory arthritis synovium |
| ITGAV | 3685 | STMN1+ proliferating cells inflammatory arthritis synovium |
| ITPA | 3704 | STMN1+ proliferating cells inflammatory arthritis synovium |
| IVNS1ABP | 10625 | STMN1+ proliferating cells inflammatory arthritis synovium |
| JPT1 | 51155 | STMN1+ proliferating cells inflammatory arthritis synovium |
| KDELR2 | 11014 | STMN1+ proliferating cells inflammatory arthritis synovium |
| KHSRP | 8570 | STMN1+ proliferating cells inflammatory arthritis synovium |
| KIAA1524 | 57650 | STMN1+ proliferating cells inflammatory arthritis synovium |
| KIF11 | 3832 | STMN1+ proliferating cells inflammatory arthritis synovium |
| KIF15 | 56992 | STMN1+ proliferating cells inflammatory arthritis synovium |
| KIF20A | 10112 | STMN1+ proliferating cells inflammatory arthritis synovium |
| KIF20B | 9585 | STMN1+ proliferating cells inflammatory arthritis synovium |
| KIF22 | 3835 | STMN1+ proliferating cells inflammatory arthritis synovium |
| KIF23 | 9493 | STMN1+ proliferating cells inflammatory arthritis synovium |
| KIF4A | 24137 | STMN1+ proliferating cells inflammatory arthritis synovium |
| KIFC1 | 3833 | STMN1+ proliferating cells inflammatory arthritis synovium |
| KLHL5 | 51088 | STMN1+ proliferating cells inflammatory arthritis synovium |
| KMT5A | 387893 | STMN1+ proliferating cells inflammatory arthritis synovium |
| KNL1 | 57082 | STMN1+ proliferating cells inflammatory arthritis synovium |
| KNSTRN | 90417 | STMN1+ proliferating cells inflammatory arthritis synovium |
| KPNA2 | 3838 | STMN1+ proliferating cells inflammatory arthritis synovium |
| KPNA3 | 3839 | STMN1+ proliferating cells inflammatory arthritis synovium |
| KPNB1 | 3837 | STMN1+ proliferating cells inflammatory arthritis synovium |
| KTN1 | 3895 | STMN1+ proliferating cells inflammatory arthritis synovium |
| LARP7 | 51574 | STMN1+ proliferating cells inflammatory arthritis synovium |
| LARS | 51520 | STMN1+ proliferating cells inflammatory arthritis synovium |
| LBR | 3930 | STMN1+ proliferating cells inflammatory arthritis synovium |
| LDHA | 3939 | STMN1+ proliferating cells inflammatory arthritis synovium |
| LIG1 | 3978 | STMN1+ proliferating cells inflammatory arthritis synovium |
| LMF2 | 91289 | STMN1+ proliferating cells inflammatory arthritis synovium |
| LMNA | 4000 | STMN1+ proliferating cells inflammatory arthritis synovium |
| LMO4 | 8543 | STMN1+ proliferating cells inflammatory arthritis synovium |
| LPL | 4023 | STMN1+ proliferating cells inflammatory arthritis synovium |
| LRPPRC | 10128 | STMN1+ proliferating cells inflammatory arthritis synovium |
| LRR1 | 122769 | STMN1+ proliferating cells inflammatory arthritis synovium |
| LSM2 | 57819 | STMN1+ proliferating cells inflammatory arthritis synovium |
| LSM3 | 27258 | STMN1+ proliferating cells inflammatory arthritis synovium |
| LSM4 | 25804 | STMN1+ proliferating cells inflammatory arthritis synovium |
| LSM5 | 23658 | STMN1+ proliferating cells inflammatory arthritis synovium |
| LSM6 | 11157 | STMN1+ proliferating cells inflammatory arthritis synovium |
| LSM7 | 51690 | STMN1+ proliferating cells inflammatory arthritis synovium |
| LSM8 | 51691 | STMN1+ proliferating cells inflammatory arthritis synovium |
| LUC7L3 | 51747 | STMN1+ proliferating cells inflammatory arthritis synovium |
| LYAR | 55646 | STMN1+ proliferating cells inflammatory arthritis synovium |
| M6PR | 4074 | STMN1+ proliferating cells inflammatory arthritis synovium |
| MAD2L1 | 4085 | STMN1+ proliferating cells inflammatory arthritis synovium |
| MAGOH | 4116 | STMN1+ proliferating cells inflammatory arthritis synovium |
| MAGOHB | 55110 | STMN1+ proliferating cells inflammatory arthritis synovium |
| MAP4K4 | 9448 | STMN1+ proliferating cells inflammatory arthritis synovium |
| MAPK1 | 5594 | STMN1+ proliferating cells inflammatory arthritis synovium |
| MATR3 | 9782 | STMN1+ proliferating cells inflammatory arthritis synovium |
| MBD3 | 53615 | STMN1+ proliferating cells inflammatory arthritis synovium |
| MCM3 | 4172 | STMN1+ proliferating cells inflammatory arthritis synovium |
| MCM4 | 4173 | STMN1+ proliferating cells inflammatory arthritis synovium |
| MCM5 | 4174 | STMN1+ proliferating cells inflammatory arthritis synovium |
| MCM6 | 4175 | STMN1+ proliferating cells inflammatory arthritis synovium |
| MCM7 | 4176 | STMN1+ proliferating cells inflammatory arthritis synovium |
| MDH2 | 4191 | STMN1+ proliferating cells inflammatory arthritis synovium |
| MED30 | 90390 | STMN1+ proliferating cells inflammatory arthritis synovium |
| MELK | 9833 | STMN1+ proliferating cells inflammatory arthritis synovium |
| METAP2 | 10988 | STMN1+ proliferating cells inflammatory arthritis synovium |
| MIF | 4282 | STMN1+ proliferating cells inflammatory arthritis synovium |
| MINOS1 | 440574 | STMN1+ proliferating cells inflammatory arthritis synovium |
| MIS12 | 79003 | STMN1+ proliferating cells inflammatory arthritis synovium |
| MIS18A | 54069 | STMN1+ proliferating cells inflammatory arthritis synovium |
| MIS18BP1 | 55320 | STMN1+ proliferating cells inflammatory arthritis synovium |
| MKI67 | 4288 | STMN1+ proliferating cells inflammatory arthritis synovium |
| MMP14 | 4323 | STMN1+ proliferating cells inflammatory arthritis synovium |
| MPHOSPH6 | 10200 | STMN1+ proliferating cells inflammatory arthritis synovium |
| MRPL12 | 6182 | STMN1+ proliferating cells inflammatory arthritis synovium |
| MRPL15 | 29088 | STMN1+ proliferating cells inflammatory arthritis synovium |
| MRPL18 | 29074 | STMN1+ proliferating cells inflammatory arthritis synovium |
| MRPL28 | 10573 | STMN1+ proliferating cells inflammatory arthritis synovium |
| MRPL34 | 64981 | STMN1+ proliferating cells inflammatory arthritis synovium |
| MRPL42 | 28977 | STMN1+ proliferating cells inflammatory arthritis synovium |
| MRPL49 | 740 | STMN1+ proliferating cells inflammatory arthritis synovium |
| MRPL51 | 51258 | STMN1+ proliferating cells inflammatory arthritis synovium |
| MRPL52 | 122704 | STMN1+ proliferating cells inflammatory arthritis synovium |
| MRPL57 | 78988 | STMN1+ proliferating cells inflammatory arthritis synovium |
| MRPS14 | 63931 | STMN1+ proliferating cells inflammatory arthritis synovium |
| MRPS17 | 51373 | STMN1+ proliferating cells inflammatory arthritis synovium |
| MRPS25 | 64432 | STMN1+ proliferating cells inflammatory arthritis synovium |
| MRPS26 | 64949 | STMN1+ proliferating cells inflammatory arthritis synovium |
| MRPS28 | 28957 | STMN1+ proliferating cells inflammatory arthritis synovium |
| MRPS36 | 92259 | STMN1+ proliferating cells inflammatory arthritis synovium |
| MT-ATP6 | 4508 | STMN1+ proliferating cells inflammatory arthritis synovium |
| MT-ATP8 | 4509 | STMN1+ proliferating cells inflammatory arthritis synovium |
| MT-CO1 | 4512 | STMN1+ proliferating cells inflammatory arthritis synovium |
| MT-CO2 | 4513 | STMN1+ proliferating cells inflammatory arthritis synovium |
| MT-CO3 | 4514 | STMN1+ proliferating cells inflammatory arthritis synovium |
| MT-ND1 | 4535 | STMN1+ proliferating cells inflammatory arthritis synovium |
| MT-ND3 | 4537 | STMN1+ proliferating cells inflammatory arthritis synovium |
| MT-ND4L | 4539 | STMN1+ proliferating cells inflammatory arthritis synovium |
| MT1A | 4489 | STMN1+ proliferating cells inflammatory arthritis synovium |
| MT2A | 4502 | STMN1+ proliferating cells inflammatory arthritis synovium |
| MXD3 | 83463 | STMN1+ proliferating cells inflammatory arthritis synovium |
| MYADM | 91663 | STMN1+ proliferating cells inflammatory arthritis synovium |
| MYBBP1A | 10514 | STMN1+ proliferating cells inflammatory arthritis synovium |
| MYBL2 | 4605 | STMN1+ proliferating cells inflammatory arthritis synovium |
| MYCBP | 26292 | STMN1+ proliferating cells inflammatory arthritis synovium |
| MYEF2 | 50804 | STMN1+ proliferating cells inflammatory arthritis synovium |
| NAA10 | 8260 | STMN1+ proliferating cells inflammatory arthritis synovium |
| NAA50 | 80218 | STMN1+ proliferating cells inflammatory arthritis synovium |
| NANS | 54187 | STMN1+ proliferating cells inflammatory arthritis synovium |
| NAP1L1 | 4673 | STMN1+ proliferating cells inflammatory arthritis synovium |
| NASP | 4678 | STMN1+ proliferating cells inflammatory arthritis synovium |
| NAV2 | 89797 | STMN1+ proliferating cells inflammatory arthritis synovium |
| NCAPD2 | 9918 | STMN1+ proliferating cells inflammatory arthritis synovium |
| NCAPD3 | 23310 | STMN1+ proliferating cells inflammatory arthritis synovium |
| NCAPG | 64151 | STMN1+ proliferating cells inflammatory arthritis synovium |
| NCAPG2 | 54892 | STMN1+ proliferating cells inflammatory arthritis synovium |
| NCAPH | 23397 | STMN1+ proliferating cells inflammatory arthritis synovium |
| NCAPH2 | 29781 | STMN1+ proliferating cells inflammatory arthritis synovium |
| NCBP1 | 4686 | STMN1+ proliferating cells inflammatory arthritis synovium |
| NCL | 4691 | STMN1+ proliferating cells inflammatory arthritis synovium |
| NDC80 | 10403 | STMN1+ proliferating cells inflammatory arthritis synovium |
| NDUFA12 | 55967 | STMN1+ proliferating cells inflammatory arthritis synovium |
| NDUFA2 | 4695 | STMN1+ proliferating cells inflammatory arthritis synovium |
| NDUFA5 | 4698 | STMN1+ proliferating cells inflammatory arthritis synovium |
| NDUFAB1 | 4706 | STMN1+ proliferating cells inflammatory arthritis synovium |
| NDUFAF2 | 91942 | STMN1+ proliferating cells inflammatory arthritis synovium |
| NDUFAF4 | 29078 | STMN1+ proliferating cells inflammatory arthritis synovium |
| NDUFB10 | 4716 | STMN1+ proliferating cells inflammatory arthritis synovium |
| NDUFB11 | 54539 | STMN1+ proliferating cells inflammatory arthritis synovium |
| NDUFB6 | 4712 | STMN1+ proliferating cells inflammatory arthritis synovium |
| NDUFB7 | 4713 | STMN1+ proliferating cells inflammatory arthritis synovium |
| NDUFB8 | 4714 | STMN1+ proliferating cells inflammatory arthritis synovium |
| NDUFC1 | 4717 | STMN1+ proliferating cells inflammatory arthritis synovium |
| NDUFC2 | 4718 | STMN1+ proliferating cells inflammatory arthritis synovium |
| NDUFS5 | 4725 | STMN1+ proliferating cells inflammatory arthritis synovium |
| NDUFV2 | 4729 | STMN1+ proliferating cells inflammatory arthritis synovium |
| NFATC1 | 4772 | STMN1+ proliferating cells inflammatory arthritis synovium |
| NHP2 | 55651 | STMN1+ proliferating cells inflammatory arthritis synovium |
| NME1 | 4830 | STMN1+ proliferating cells inflammatory arthritis synovium |
| NMRAL1 | 57407 | STMN1+ proliferating cells inflammatory arthritis synovium |
| NMT2 | 9397 | STMN1+ proliferating cells inflammatory arthritis synovium |
| NOL7 | 51406 | STMN1+ proliferating cells inflammatory arthritis synovium |
| NOLC1 | 9221 | STMN1+ proliferating cells inflammatory arthritis synovium |
| NONO | 4841 | STMN1+ proliferating cells inflammatory arthritis synovium |
| NOP10 | 55505 | STMN1+ proliferating cells inflammatory arthritis synovium |
| NOP16 | 51491 | STMN1+ proliferating cells inflammatory arthritis synovium |
| NOP56 | 10528 | STMN1+ proliferating cells inflammatory arthritis synovium |
| NOP58 | 51602 | STMN1+ proliferating cells inflammatory arthritis synovium |
| NPM1 | 4869 | STMN1+ proliferating cells inflammatory arthritis synovium |
| NPM3 | 10360 | STMN1+ proliferating cells inflammatory arthritis synovium |
| NRM | 11270 | STMN1+ proliferating cells inflammatory arthritis synovium |
| NRP2 | 8828 | STMN1+ proliferating cells inflammatory arthritis synovium |
| NSD2 | 7468 | STMN1+ proliferating cells inflammatory arthritis synovium |
| NSMCE1 | 197370 | STMN1+ proliferating cells inflammatory arthritis synovium |
| NSUN2 | 54888 | STMN1+ proliferating cells inflammatory arthritis synovium |
| NUBP1 | 4682 | STMN1+ proliferating cells inflammatory arthritis synovium |
| NUCKS1 | 64710 | STMN1+ proliferating cells inflammatory arthritis synovium |
| NUDC | 10726 | STMN1+ proliferating cells inflammatory arthritis synovium |
| NUDCD2 | 134492 | STMN1+ proliferating cells inflammatory arthritis synovium |
| NUDT5 | 11164 | STMN1+ proliferating cells inflammatory arthritis synovium |
| NUF2 | 83540 | STMN1+ proliferating cells inflammatory arthritis synovium |
| NUMA1 | 4926 | STMN1+ proliferating cells inflammatory arthritis synovium |
| NUP35 | 129401 | STMN1+ proliferating cells inflammatory arthritis synovium |
| NUP85 | 79902 | STMN1+ proliferating cells inflammatory arthritis synovium |
| NUP93 | 9688 | STMN1+ proliferating cells inflammatory arthritis synovium |
| NUSAP1 | 51203 | STMN1+ proliferating cells inflammatory arthritis synovium |
| NXT1 | 29107 | STMN1+ proliferating cells inflammatory arthritis synovium |
| ODC1 | 4953 | STMN1+ proliferating cells inflammatory arthritis synovium |
| ODF2 | 4957 | STMN1+ proliferating cells inflammatory arthritis synovium |
| OLA1 | 29789 | STMN1+ proliferating cells inflammatory arthritis synovium |
| ORC6 | 23594 | STMN1+ proliferating cells inflammatory arthritis synovium |
| OXCT1 | 5019 | STMN1+ proliferating cells inflammatory arthritis synovium |
| PA2G4 | 5036 | STMN1+ proliferating cells inflammatory arthritis synovium |
| PABPC1 | 26986 | STMN1+ proliferating cells inflammatory arthritis synovium |
| PABPC4 | 8761 | STMN1+ proliferating cells inflammatory arthritis synovium |
| PAICS | 10606 | STMN1+ proliferating cells inflammatory arthritis synovium |
| PARK7 | 11315 | STMN1+ proliferating cells inflammatory arthritis synovium |
| PARL | 55486 | STMN1+ proliferating cells inflammatory arthritis synovium |
| PBK | 55872 | STMN1+ proliferating cells inflammatory arthritis synovium |
| PBRM1 | 55193 | STMN1+ proliferating cells inflammatory arthritis synovium |
| PCBD2 | 84105 | STMN1+ proliferating cells inflammatory arthritis synovium |
| PCBP1 | 5093 | STMN1+ proliferating cells inflammatory arthritis synovium |
| PCBP2 | 5094 | STMN1+ proliferating cells inflammatory arthritis synovium |
| PCLAF | 9768 | STMN1+ proliferating cells inflammatory arthritis synovium |
| PCNA | 5111 | STMN1+ proliferating cells inflammatory arthritis synovium |
| PDAP1 | 11333 | STMN1+ proliferating cells inflammatory arthritis synovium |
| PDCD5 | 9141 | STMN1+ proliferating cells inflammatory arthritis synovium |
| PDPK1 | 5170 | STMN1+ proliferating cells inflammatory arthritis synovium |
| PDS5A | 23244 | STMN1+ proliferating cells inflammatory arthritis synovium |
| PFDN4 | 5203 | STMN1+ proliferating cells inflammatory arthritis synovium |
| PFDN6 | 10471 | STMN1+ proliferating cells inflammatory arthritis synovium |
| PGK1 | 5230 | STMN1+ proliferating cells inflammatory arthritis synovium |
| PGP | 283871 | STMN1+ proliferating cells inflammatory arthritis synovium |
| PHB2 | 11331 | STMN1+ proliferating cells inflammatory arthritis synovium |
| PIH1D1 | 55011 | STMN1+ proliferating cells inflammatory arthritis synovium |
| PIMREG | 54478 | STMN1+ proliferating cells inflammatory arthritis synovium |
| PKMYT1 | 9088 | STMN1+ proliferating cells inflammatory arthritis synovium |
| PLK1 | 5347 | STMN1+ proliferating cells inflammatory arthritis synovium |
| PLK4 | 10733 | STMN1+ proliferating cells inflammatory arthritis synovium |
| PLP2 | 5355 | STMN1+ proliferating cells inflammatory arthritis synovium |
| PMF1 | 11243 | STMN1+ proliferating cells inflammatory arthritis synovium |
| PNN | 5411 | STMN1+ proliferating cells inflammatory arthritis synovium |
| POLD2 | 5425 | STMN1+ proliferating cells inflammatory arthritis synovium |
| POLE3 | 54107 | STMN1+ proliferating cells inflammatory arthritis synovium |
| POLE4 | 56655 | STMN1+ proliferating cells inflammatory arthritis synovium |
| POLR2A | 5430 | STMN1+ proliferating cells inflammatory arthritis synovium |
| POLR2F | 5435 | STMN1+ proliferating cells inflammatory arthritis synovium |
| POLR2L | 5441 | STMN1+ proliferating cells inflammatory arthritis synovium |
| PPA1 | 5464 | STMN1+ proliferating cells inflammatory arthritis synovium |
| PPIA | 5478 | STMN1+ proliferating cells inflammatory arthritis synovium |
| PPID | 5481 | STMN1+ proliferating cells inflammatory arthritis synovium |
| PPIH | 10465 | STMN1+ proliferating cells inflammatory arthritis synovium |
| PPP2R5C | 5527 | STMN1+ proliferating cells inflammatory arthritis synovium |
| PRC1 | 9055 | STMN1+ proliferating cells inflammatory arthritis synovium |
| PRDX4 | 10549 | STMN1+ proliferating cells inflammatory arthritis synovium |
| PRIM1 | 5557 | STMN1+ proliferating cells inflammatory arthritis synovium |
| PRKAR2A | 5576 | STMN1+ proliferating cells inflammatory arthritis synovium |
| PRMT1 | 3276 | STMN1+ proliferating cells inflammatory arthritis synovium |
| PRR11 | 55771 | STMN1+ proliferating cells inflammatory arthritis synovium |
| PRRC2A | 7916 | STMN1+ proliferating cells inflammatory arthritis synovium |
| PSAT1 | 29968 | STMN1+ proliferating cells inflammatory arthritis synovium |
| PSIP1 | 11168 | STMN1+ proliferating cells inflammatory arthritis synovium |
| PSMA6 | 5687 | STMN1+ proliferating cells inflammatory arthritis synovium |
| PSMB7 | 5695 | STMN1+ proliferating cells inflammatory arthritis synovium |
| PSMC3 | 5702 | STMN1+ proliferating cells inflammatory arthritis synovium |
| PSMD1 | 5707 | STMN1+ proliferating cells inflammatory arthritis synovium |
| PSMD10 | 5716 | STMN1+ proliferating cells inflammatory arthritis synovium |
| PSMD14 | 10213 | STMN1+ proliferating cells inflammatory arthritis synovium |
| PSMG2 | 56984 | STMN1+ proliferating cells inflammatory arthritis synovium |
| PTBP1 | 5725 | STMN1+ proliferating cells inflammatory arthritis synovium |
| PTGES3 | 10728 | STMN1+ proliferating cells inflammatory arthritis synovium |
| PTMA | 5757 | STMN1+ proliferating cells inflammatory arthritis synovium |
| PTPN7 | 5778 | STMN1+ proliferating cells inflammatory arthritis synovium |
| PURB | 5814 | STMN1+ proliferating cells inflammatory arthritis synovium |
| QDPR | 5860 | STMN1+ proliferating cells inflammatory arthritis synovium |
| RACGAP1 | 29127 | STMN1+ proliferating cells inflammatory arthritis synovium |
| RACK1 | 10399 | STMN1+ proliferating cells inflammatory arthritis synovium |
| RAD21 | 5885 | STMN1+ proliferating cells inflammatory arthritis synovium |
| RAD23B | 5887 | STMN1+ proliferating cells inflammatory arthritis synovium |
| RAD51 | 5888 | STMN1+ proliferating cells inflammatory arthritis synovium |
| RAD51AP1 | 10635 | STMN1+ proliferating cells inflammatory arthritis synovium |
| RAD54L | 8438 | STMN1+ proliferating cells inflammatory arthritis synovium |
| RAN | 5901 | STMN1+ proliferating cells inflammatory arthritis synovium |
| RANBP1 | 5902 | STMN1+ proliferating cells inflammatory arthritis synovium |
| RANGAP1 | 5905 | STMN1+ proliferating cells inflammatory arthritis synovium |
| RBBP4 | 5928 | STMN1+ proliferating cells inflammatory arthritis synovium |
| RBBP7 | 5931 | STMN1+ proliferating cells inflammatory arthritis synovium |
| RBM3 | 5935 | STMN1+ proliferating cells inflammatory arthritis synovium |
| RBM8A | 9939 | STMN1+ proliferating cells inflammatory arthritis synovium |
| RCC1 | 1104 | STMN1+ proliferating cells inflammatory arthritis synovium |
| RCC2 | 55920 | STMN1+ proliferating cells inflammatory arthritis synovium |
| RDM1 | 201299 | STMN1+ proliferating cells inflammatory arthritis synovium |
| RDX | 5962 | STMN1+ proliferating cells inflammatory arthritis synovium |
| RFC1 | 5981 | STMN1+ proliferating cells inflammatory arthritis synovium |
| RFC2 | 5982 | STMN1+ proliferating cells inflammatory arthritis synovium |
| RFC3 | 5983 | STMN1+ proliferating cells inflammatory arthritis synovium |
| RFC4 | 5984 | STMN1+ proliferating cells inflammatory arthritis synovium |
| RFC5 | 5985 | STMN1+ proliferating cells inflammatory arthritis synovium |
| RFWD3 | 55159 | STMN1+ proliferating cells inflammatory arthritis synovium |
| RHNO1 | 83695 | STMN1+ proliferating cells inflammatory arthritis synovium |
| RNASEH2A | 10535 | STMN1+ proliferating cells inflammatory arthritis synovium |
| RNASEH2B | 79621 | STMN1+ proliferating cells inflammatory arthritis synovium |
| RNASEH2C | 84153 | STMN1+ proliferating cells inflammatory arthritis synovium |
| RNF19B | 127544 | STMN1+ proliferating cells inflammatory arthritis synovium |
| RNF7 | 9616 | STMN1+ proliferating cells inflammatory arthritis synovium |
| RNPS1 | 10921 | STMN1+ proliferating cells inflammatory arthritis synovium |
| ROCK2 | 9475 | STMN1+ proliferating cells inflammatory arthritis synovium |
| RPA1 | 6117 | STMN1+ proliferating cells inflammatory arthritis synovium |
| RPA2 | 6118 | STMN1+ proliferating cells inflammatory arthritis synovium |
| RPA3 | 6119 | STMN1+ proliferating cells inflammatory arthritis synovium |
| RPL10A | 4736 | STMN1+ proliferating cells inflammatory arthritis synovium |
| RPL12 | 6136 | STMN1+ proliferating cells inflammatory arthritis synovium |
| RPL13A | 23521 | STMN1+ proliferating cells inflammatory arthritis synovium |
| RPL14 | 9045 | STMN1+ proliferating cells inflammatory arthritis synovium |
| RPL18 | 6141 | STMN1+ proliferating cells inflammatory arthritis synovium |
| RPL21 | 6144 | STMN1+ proliferating cells inflammatory arthritis synovium |
| RPL22 | 6146 | STMN1+ proliferating cells inflammatory arthritis synovium |
| RPL22L1 | 200916 | STMN1+ proliferating cells inflammatory arthritis synovium |
| RPL23 | 9349 | STMN1+ proliferating cells inflammatory arthritis synovium |
| RPL28 | 6158 | STMN1+ proliferating cells inflammatory arthritis synovium |
| RPL30 | 6156 | STMN1+ proliferating cells inflammatory arthritis synovium |
| RPL31 | 6160 | STMN1+ proliferating cells inflammatory arthritis synovium |
| RPL32 | 6161 | STMN1+ proliferating cells inflammatory arthritis synovium |
| RPL35 | 11224 | STMN1+ proliferating cells inflammatory arthritis synovium |
| RPL35A | 6165 | STMN1+ proliferating cells inflammatory arthritis synovium |
| RPL36 | 25873 | STMN1+ proliferating cells inflammatory arthritis synovium |
| RPL36A | 6173 | STMN1+ proliferating cells inflammatory arthritis synovium |
| RPL37A | 6168 | STMN1+ proliferating cells inflammatory arthritis synovium |
| RPL38 | 6169 | STMN1+ proliferating cells inflammatory arthritis synovium |
| RPL39 | 6170 | STMN1+ proliferating cells inflammatory arthritis synovium |
| RPL5 | 6125 | STMN1+ proliferating cells inflammatory arthritis synovium |
| RPLP1 | 6176 | STMN1+ proliferating cells inflammatory arthritis synovium |
| RPLP2 | 6181 | STMN1+ proliferating cells inflammatory arthritis synovium |
| RPS10 | 6204 | STMN1+ proliferating cells inflammatory arthritis synovium |
| RPS12 | 6206 | STMN1+ proliferating cells inflammatory arthritis synovium |
| RPS13 | 6207 | STMN1+ proliferating cells inflammatory arthritis synovium |
| RPS15 | 6209 | STMN1+ proliferating cells inflammatory arthritis synovium |
| RPS15A | 6210 | STMN1+ proliferating cells inflammatory arthritis synovium |
| RPS17 | 6218 | STMN1+ proliferating cells inflammatory arthritis synovium |
| RPS18 | 6222 | STMN1+ proliferating cells inflammatory arthritis synovium |
| RPS19 | 6223 | STMN1+ proliferating cells inflammatory arthritis synovium |
| RPS19BP1 | 91582 | STMN1+ proliferating cells inflammatory arthritis synovium |
| RPS2 | 6187 | STMN1+ proliferating cells inflammatory arthritis synovium |
| RPS20 | 6224 | STMN1+ proliferating cells inflammatory arthritis synovium |
| RPS21 | 6227 | STMN1+ proliferating cells inflammatory arthritis synovium |
| RPS24 | 6229 | STMN1+ proliferating cells inflammatory arthritis synovium |
| RPS26 | 6231 | STMN1+ proliferating cells inflammatory arthritis synovium |
| RPS27A | 6233 | STMN1+ proliferating cells inflammatory arthritis synovium |
| RPS27L | 51065 | STMN1+ proliferating cells inflammatory arthritis synovium |
| RPS3A | 6189 | STMN1+ proliferating cells inflammatory arthritis synovium |
| RPS5 | 6193 | STMN1+ proliferating cells inflammatory arthritis synovium |
| RPS8 | 6202 | STMN1+ proliferating cells inflammatory arthritis synovium |
| RPSA | 3921 | STMN1+ proliferating cells inflammatory arthritis synovium |
| RRM1 | 6240 | STMN1+ proliferating cells inflammatory arthritis synovium |
| RRM2 | 6241 | STMN1+ proliferating cells inflammatory arthritis synovium |
| RSL1D1 | 26156 | STMN1+ proliferating cells inflammatory arthritis synovium |
| S100A10 | 6281 | STMN1+ proliferating cells inflammatory arthritis synovium |
| S100A4 | 6275 | STMN1+ proliferating cells inflammatory arthritis synovium |
| SAE1 | 10055 | STMN1+ proliferating cells inflammatory arthritis synovium |
| SDC3 | 9672 | STMN1+ proliferating cells inflammatory arthritis synovium |
| SDHD | 6392 | STMN1+ proliferating cells inflammatory arthritis synovium |
| SEC11C | 90701 | STMN1+ proliferating cells inflammatory arthritis synovium |
| SEC31A | 22872 | STMN1+ proliferating cells inflammatory arthritis synovium |
| SELENOH | 280636 | STMN1+ proliferating cells inflammatory arthritis synovium |
| SERBP1 | 26135 | STMN1+ proliferating cells inflammatory arthritis synovium |
| SET | 6418 | STMN1+ proliferating cells inflammatory arthritis synovium |
| SF3A3 | 10946 | STMN1+ proliferating cells inflammatory arthritis synovium |
| SF3B3 | 23450 | STMN1+ proliferating cells inflammatory arthritis synovium |
| SF3B5 | 83443 | STMN1+ proliferating cells inflammatory arthritis synovium |
| SFPQ | 6421 | STMN1+ proliferating cells inflammatory arthritis synovium |
| SFXN1 | 94081 | STMN1+ proliferating cells inflammatory arthritis synovium |
| SGO1 | 151648 | STMN1+ proliferating cells inflammatory arthritis synovium |
| SGO2 | 151246 | STMN1+ proliferating cells inflammatory arthritis synovium |
| SHCBP1 | 79801 | STMN1+ proliferating cells inflammatory arthritis synovium |
| SHMT2 | 6472 | STMN1+ proliferating cells inflammatory arthritis synovium |
| SIVA1 | 10572 | STMN1+ proliferating cells inflammatory arthritis synovium |
| SKA1 | 220134 | STMN1+ proliferating cells inflammatory arthritis synovium |
| SLBP | 7884 | STMN1+ proliferating cells inflammatory arthritis synovium |
| SLC1A5 | 6510 | STMN1+ proliferating cells inflammatory arthritis synovium |
| SLC25A10 | 1468 | STMN1+ proliferating cells inflammatory arthritis synovium |
| SLC25A4 | 291 | STMN1+ proliferating cells inflammatory arthritis synovium |
| SLC25A5 | 292 | STMN1+ proliferating cells inflammatory arthritis synovium |
| SLC29A1 | 2030 | STMN1+ proliferating cells inflammatory arthritis synovium |
| SLC4A7 | 9497 | STMN1+ proliferating cells inflammatory arthritis synovium |
| SLFN13 | 146857 | STMN1+ proliferating cells inflammatory arthritis synovium |
| SLIRP | 81892 | STMN1+ proliferating cells inflammatory arthritis synovium |
| SMARCA5 | 8467 | STMN1+ proliferating cells inflammatory arthritis synovium |
| SMC1A | 8243 | STMN1+ proliferating cells inflammatory arthritis synovium |
| SMC2 | 10592 | STMN1+ proliferating cells inflammatory arthritis synovium |
| SMC3 | 9126 | STMN1+ proliferating cells inflammatory arthritis synovium |
| SMC4 | 10051 | STMN1+ proliferating cells inflammatory arthritis synovium |
| SMC6 | 79677 | STMN1+ proliferating cells inflammatory arthritis synovium |
| SMCHD1 | 23347 | STMN1+ proliferating cells inflammatory arthritis synovium |
| SMS | 6611 | STMN1+ proliferating cells inflammatory arthritis synovium |
| SMYD2 | 56950 | STMN1+ proliferating cells inflammatory arthritis synovium |
| SNHG6 | 641638 | STMN1+ proliferating cells inflammatory arthritis synovium |
| SNRNP25 | 79622 | STMN1+ proliferating cells inflammatory arthritis synovium |
| SNRNP27 | 11017 | STMN1+ proliferating cells inflammatory arthritis synovium |
| SNRNP70 | 6625 | STMN1+ proliferating cells inflammatory arthritis synovium |
| SNRPA1 | 6627 | STMN1+ proliferating cells inflammatory arthritis synovium |
| SNRPB | 6628 | STMN1+ proliferating cells inflammatory arthritis synovium |
| SNRPC | 6631 | STMN1+ proliferating cells inflammatory arthritis synovium |
| SNRPD1 | 6632 | STMN1+ proliferating cells inflammatory arthritis synovium |
| SNRPD2 | 6633 | STMN1+ proliferating cells inflammatory arthritis synovium |
| SNRPD3 | 6634 | STMN1+ proliferating cells inflammatory arthritis synovium |
| SNRPE | 6635 | STMN1+ proliferating cells inflammatory arthritis synovium |
| SNRPF | 6636 | STMN1+ proliferating cells inflammatory arthritis synovium |
| SNRPG | 6637 | STMN1+ proliferating cells inflammatory arthritis synovium |
| SNU13 | 4809 | STMN1+ proliferating cells inflammatory arthritis synovium |
| SOD1 | 6647 | STMN1+ proliferating cells inflammatory arthritis synovium |
| SPC24 | 147841 | STMN1+ proliferating cells inflammatory arthritis synovium |
| SPC25 | 57405 | STMN1+ proliferating cells inflammatory arthritis synovium |
| SPCS3 | 60559 | STMN1+ proliferating cells inflammatory arthritis synovium |
| SPN | 6693 | STMN1+ proliferating cells inflammatory arthritis synovium |
| SRM | 6723 | STMN1+ proliferating cells inflammatory arthritis synovium |
| SRRM1 | 10250 | STMN1+ proliferating cells inflammatory arthritis synovium |
| SRSF1 | 6426 | STMN1+ proliferating cells inflammatory arthritis synovium |
| SRSF10 | 10772 | STMN1+ proliferating cells inflammatory arthritis synovium |
| SRSF2 | 6427 | STMN1+ proliferating cells inflammatory arthritis synovium |
| SRSF3 | 6428 | STMN1+ proliferating cells inflammatory arthritis synovium |
| SRSF7 | 6432 | STMN1+ proliferating cells inflammatory arthritis synovium |
| SSB | 6741 | STMN1+ proliferating cells inflammatory arthritis synovium |
| SSNA1 | 8636 | STMN1+ proliferating cells inflammatory arthritis synovium |
| SSRP1 | 6749 | STMN1+ proliferating cells inflammatory arthritis synovium |
| STIP1 | 10963 | STMN1+ proliferating cells inflammatory arthritis synovium |
| STMN1 | 3925 | STMN1+ proliferating cells inflammatory arthritis synovium |
| STUB1 | 10273 | STMN1+ proliferating cells inflammatory arthritis synovium |
| SUPT16H | 11198 | STMN1+ proliferating cells inflammatory arthritis synovium |
| SUZ12 | 23512 | STMN1+ proliferating cells inflammatory arthritis synovium |
| SYCE2 | 256126 | STMN1+ proliferating cells inflammatory arthritis synovium |
| SYNCRIP | 10492 | STMN1+ proliferating cells inflammatory arthritis synovium |
| TACC3 | 10460 | STMN1+ proliferating cells inflammatory arthritis synovium |
| TAGLN2 | 8407 | STMN1+ proliferating cells inflammatory arthritis synovium |
| TARDBP | 23435 | STMN1+ proliferating cells inflammatory arthritis synovium |
| TCEAL9 | 51186 | STMN1+ proliferating cells inflammatory arthritis synovium |
| TCF3 | 6929 | STMN1+ proliferating cells inflammatory arthritis synovium |
| TCP1 | 6950 | STMN1+ proliferating cells inflammatory arthritis synovium |
| TEX30 | 93081 | STMN1+ proliferating cells inflammatory arthritis synovium |
| TFDP1 | 7027 | STMN1+ proliferating cells inflammatory arthritis synovium |
| TFRC | 7037 | STMN1+ proliferating cells inflammatory arthritis synovium |
| THOC7 | 80145 | STMN1+ proliferating cells inflammatory arthritis synovium |
| TIAL1 | 7073 | STMN1+ proliferating cells inflammatory arthritis synovium |
| TIMM13 | 26517 | STMN1+ proliferating cells inflammatory arthritis synovium |
| TIMM50 | 92609 | STMN1+ proliferating cells inflammatory arthritis synovium |
| TIMM8A | 1678 | STMN1+ proliferating cells inflammatory arthritis synovium |
| TIPIN | 54962 | STMN1+ proliferating cells inflammatory arthritis synovium |
| TK1 | 7083 | STMN1+ proliferating cells inflammatory arthritis synovium |
| TMEM107 | 84314 | STMN1+ proliferating cells inflammatory arthritis synovium |
| TMEM97 | 27346 | STMN1+ proliferating cells inflammatory arthritis synovium |
| TMPO | 7112 | STMN1+ proliferating cells inflammatory arthritis synovium |
| TOMM20 | 9804 | STMN1+ proliferating cells inflammatory arthritis synovium |
| TOMM5 | 401505 | STMN1+ proliferating cells inflammatory arthritis synovium |
| TOMM7 | 54543 | STMN1+ proliferating cells inflammatory arthritis synovium |
| TOMM70A | 9868 | STMN1+ proliferating cells inflammatory arthritis synovium |
| TOP2A | 7153 | STMN1+ proliferating cells inflammatory arthritis synovium |
| TOPBP1 | 11073 | STMN1+ proliferating cells inflammatory arthritis synovium |
| TPM4 | 7171 | STMN1+ proliferating cells inflammatory arthritis synovium |
| TPX2 | 22974 | STMN1+ proliferating cells inflammatory arthritis synovium |
| TRIM28 | 10155 | STMN1+ proliferating cells inflammatory arthritis synovium |
| TRIM59 | 286827 | STMN1+ proliferating cells inflammatory arthritis synovium |
| TRIP13 | 9319 | STMN1+ proliferating cells inflammatory arthritis synovium |
| TSC22D1 | 8848 | STMN1+ proliferating cells inflammatory arthritis synovium |
| TSFM | 10102 | STMN1+ proliferating cells inflammatory arthritis synovium |
| TSSC4 | 10078 | STMN1+ proliferating cells inflammatory arthritis synovium |
| TUBA1B | 10376 | STMN1+ proliferating cells inflammatory arthritis synovium |
| TUBA1C | 84790 | STMN1+ proliferating cells inflammatory arthritis synovium |
| TUBB | 203068 | STMN1+ proliferating cells inflammatory arthritis synovium |
| TUBB2A | 7280 | STMN1+ proliferating cells inflammatory arthritis synovium |
| TUBB4B | 10383 | STMN1+ proliferating cells inflammatory arthritis synovium |
| TUBB6 | 84617 | STMN1+ proliferating cells inflammatory arthritis synovium |
| TUBG1 | 7283 | STMN1+ proliferating cells inflammatory arthritis synovium |
| TXN | 7295 | STMN1+ proliferating cells inflammatory arthritis synovium |
| TXNL1 | 9352 | STMN1+ proliferating cells inflammatory arthritis synovium |
| TXNRD1 | 7296 | STMN1+ proliferating cells inflammatory arthritis synovium |
| TYMS | 7298 | STMN1+ proliferating cells inflammatory arthritis synovium |
| U2AF1 | 7307 | STMN1+ proliferating cells inflammatory arthritis synovium |
| UBA2 | 10054 | STMN1+ proliferating cells inflammatory arthritis synovium |
| UBALD2 | 283991 | STMN1+ proliferating cells inflammatory arthritis synovium |
| UBE2C | 11065 | STMN1+ proliferating cells inflammatory arthritis synovium |
| UBE2S | 27338 | STMN1+ proliferating cells inflammatory arthritis synovium |
| UBE2T | 29089 | STMN1+ proliferating cells inflammatory arthritis synovium |
| UCHL5 | 51377 | STMN1+ proliferating cells inflammatory arthritis synovium |
| UHRF1 | 29128 | STMN1+ proliferating cells inflammatory arthritis synovium |
| UQCC2 | 84300 | STMN1+ proliferating cells inflammatory arthritis synovium |
| UQCR10 | 29796 | STMN1+ proliferating cells inflammatory arthritis synovium |
| UQCR11 | 10975 | STMN1+ proliferating cells inflammatory arthritis synovium |
| UQCRFS1 | 7386 | STMN1+ proliferating cells inflammatory arthritis synovium |
| UQCRQ | 27089 | STMN1+ proliferating cells inflammatory arthritis synovium |
| USMG5 | 84833 | STMN1+ proliferating cells inflammatory arthritis synovium |
| USP1 | 7398 | STMN1+ proliferating cells inflammatory arthritis synovium |
| VARS | 7407 | STMN1+ proliferating cells inflammatory arthritis synovium |
| VCP | 7415 | STMN1+ proliferating cells inflammatory arthritis synovium |
| VDAC1 | 7416 | STMN1+ proliferating cells inflammatory arthritis synovium |
| VDAC2 | 7417 | STMN1+ proliferating cells inflammatory arthritis synovium |
| VIM | 7431 | STMN1+ proliferating cells inflammatory arthritis synovium |
| VMA21 | 203547 | STMN1+ proliferating cells inflammatory arthritis synovium |
| VRK1 | 7443 | STMN1+ proliferating cells inflammatory arthritis synovium |
| WAPL | 23063 | STMN1+ proliferating cells inflammatory arthritis synovium |
| WDR43 | 23160 | STMN1+ proliferating cells inflammatory arthritis synovium |
| WDR89 | 112840 | STMN1+ proliferating cells inflammatory arthritis synovium |
| XPO1 | 7514 | STMN1+ proliferating cells inflammatory arthritis synovium |
| YBX1 | 4904 | STMN1+ proliferating cells inflammatory arthritis synovium |
| YBX3 | 8531 | STMN1+ proliferating cells inflammatory arthritis synovium |
| YWHAH | 7533 | STMN1+ proliferating cells inflammatory arthritis synovium |
| ZC3H15 | 55854 | STMN1+ proliferating cells inflammatory arthritis synovium |
| ADAM28 | 10863 | HBEGF+ proinflammatory macrophages |
| ADAMDEC1 | 27299 | HBEGF+ proinflammatory macrophages |
| ANKRD37 | 353322 | HBEGF+ proinflammatory macrophages |
| ARL4A | 10124 | HBEGF+ proinflammatory macrophages |
| ARL5B | 221079 | HBEGF+ proinflammatory macrophages |
| ATF3 | 467 | HBEGF+ proinflammatory macrophages |
| B4GALT1 | 2683 | HBEGF+ proinflammatory macrophages |
| BIRC3 | 330 | HBEGF+ proinflammatory macrophages |
| CCNH | 902 | HBEGF+ proinflammatory macrophages |
| CHML | 1122 | HBEGF+ proinflammatory macrophages |
| CHMP1B | 57132 | HBEGF+ proinflammatory macrophages |
| CREM | 1390 | HBEGF+ proinflammatory macrophages |
| CSRNP1 | 64651 | HBEGF+ proinflammatory macrophages |
| CXCL2 | 2920 | HBEGF+ proinflammatory macrophages |
| CXCL3 | 2921 | HBEGF+ proinflammatory macrophages |
| CXCL8 | 3576 | HBEGF+ proinflammatory macrophages |
| CYB5D1 | 124637 | HBEGF+ proinflammatory macrophages |
| DDX3Y | 8653 | HBEGF+ proinflammatory macrophages |
| DNAJA4 | 55466 | HBEGF+ proinflammatory macrophages |
| DNAJB1 | 3337 | HBEGF+ proinflammatory macrophages |
| DNAJB6 | 10049 | HBEGF+ proinflammatory macrophages |
| DUSP2 | 1844 | HBEGF+ proinflammatory macrophages |
| EGR1 | 1958 | HBEGF+ proinflammatory macrophages |
| EGR3 | 1960 | HBEGF+ proinflammatory macrophages |
| EREG | 2069 | HBEGF+ proinflammatory macrophages |
| ERN1 | 2081 | HBEGF+ proinflammatory macrophages |
| FCER1A | 2205 | HBEGF+ proinflammatory macrophages |
| FOSB | 2354 | HBEGF+ proinflammatory macrophages |
| FOSL2 | 2355 | HBEGF+ proinflammatory macrophages |
| GLA | 2717 | HBEGF+ proinflammatory macrophages |
| GPR183 | 1880 | HBEGF+ proinflammatory macrophages |
| GRASP | 160622 | HBEGF+ proinflammatory macrophages |
| HBEGF | 1839 | HBEGF+ proinflammatory macrophages |
| HNRNPU-AS1 | 284702 | HBEGF+ proinflammatory macrophages |
| HSP90AA1 | 3320 | HBEGF+ proinflammatory macrophages |
| HSPA1A | 3303 | HBEGF+ proinflammatory macrophages |
| HSPA1B | 3304 | HBEGF+ proinflammatory macrophages |
| HSPD1 | 3329 | HBEGF+ proinflammatory macrophages |
| HSPH1 | 10808 | HBEGF+ proinflammatory macrophages |
| IER2 | 9592 | HBEGF+ proinflammatory macrophages |
| IER3 | 8870 | HBEGF+ proinflammatory macrophages |
| IL1B | 3553 | HBEGF+ proinflammatory macrophages |
| INSIG1 | 3638 | HBEGF+ proinflammatory macrophages |
| JMY | 133746 | HBEGF+ proinflammatory macrophages |
| JUNB | 3726 | HBEGF+ proinflammatory macrophages |
| KDM6B | 23135 | HBEGF+ proinflammatory macrophages |
| LDLR | 3949 | HBEGF+ proinflammatory macrophages |
| MAFF | 23764 | HBEGF+ proinflammatory macrophages |
| MIR24-2 | 407013 | HBEGF+ proinflammatory macrophages |
| MIR29B1 | 407024 | HBEGF+ proinflammatory macrophages |
| MXD1 | 4084 | HBEGF+ proinflammatory macrophages |
| NFIL3 | 4783 | HBEGF+ proinflammatory macrophages |
| NFKBID | 84807 | HBEGF+ proinflammatory macrophages |
| NR4A1 | 3164 | HBEGF+ proinflammatory macrophages |
| NR4A2 | 4929 | HBEGF+ proinflammatory macrophages |
| NR4A3 | 8013 | HBEGF+ proinflammatory macrophages |
| NRARP | 441478 | HBEGF+ proinflammatory macrophages |
| PFKFB3 | 5209 | HBEGF+ proinflammatory macrophages |
| PHACTR1 | 221692 | HBEGF+ proinflammatory macrophages |
| PLAUR | 5329 | HBEGF+ proinflammatory macrophages |
| PRMT9 | 90826 | HBEGF+ proinflammatory macrophages |
| PTGER4 | 5734 | HBEGF+ proinflammatory macrophages |
| PTGS2 | 5743 | HBEGF+ proinflammatory macrophages |
| RASSF5 | 83593 | HBEGF+ proinflammatory macrophages |
| RGS2 | 5997 | HBEGF+ proinflammatory macrophages |
| RHOB | 388 | HBEGF+ proinflammatory macrophages |
| RN7SL368P | 1.06E+08 | HBEGF+ proinflammatory macrophages |
| SATB1 | 6304 | HBEGF+ proinflammatory macrophages |
| SELK | 58515 | HBEGF+ proinflammatory macrophages |
| SEMA4A | 64218 | HBEGF+ proinflammatory macrophages |
| SIK1 | 150094 | HBEGF+ proinflammatory macrophages |
| SLC25A44 | 9673 | HBEGF+ proinflammatory macrophages |
| SOCS3 | 9021 | HBEGF+ proinflammatory macrophages |
| SYAP1 | 94056 | HBEGF+ proinflammatory macrophages |
| THBS1 | 7057 | HBEGF+ proinflammatory macrophages |
| TNFAIP3 | 7128 | HBEGF+ proinflammatory macrophages |
| TSPYL2 | 64061 | HBEGF+ proinflammatory macrophages |
| USP36 | 57602 | HBEGF+ proinflammatory macrophages |
| VEGFA | 7422 | HBEGF+ proinflammatory macrophages |
| ZFP36 | 7538 | HBEGF+ proinflammatory macrophages |
| ZNF331 | 55422 | HBEGF+ proinflammatory macrophages |
| ALDOA | 226 | IFN-activated macrophages |
| APOBEC3A | 200315 | IFN-activated macrophages |
| AQP9 | 366 | IFN-activated macrophages |
| C15orf48 | 84419 | IFN-activated macrophages |
| CD300E | 342510 | IFN-activated macrophages |
| CD36 | 948 | IFN-activated macrophages |
| CD52 | 1043 | IFN-activated macrophages |
| CSTB | 1476 | IFN-activated macrophages |
| ENO1 | 2023 | IFN-activated macrophages |
| ENO2 | 2026 | IFN-activated macrophages |
| FAM65B | 9750 | IFN-activated macrophages |
| FCN1 | 2219 | IFN-activated macrophages |
| FN1 | 2335 | IFN-activated macrophages |
| GAPDH | 2597 | IFN-activated macrophages |
| IFI44L | 10964 | IFN-activated macrophages |
| IFI6 | 2537 | IFN-activated macrophages |
| IGHM | 3507 | IFN-activated macrophages |
| ISG15 | 9636 | IFN-activated macrophages |
| ITGB7 | 3695 | IFN-activated macrophages |
| LPL | 4023 | IFN-activated macrophages |
| LY6E | 4061 | IFN-activated macrophages |
| MT1E | 4493 | IFN-activated macrophages |
| MT1F | 4494 | IFN-activated macrophages |
| MT1G | 4495 | IFN-activated macrophages |
| MT1X | 4501 | IFN-activated macrophages |
| MT2A | 4502 | IFN-activated macrophages |
| OPTN | 10133 | IFN-activated macrophages |
| S100A8 | 6279 | IFN-activated macrophages |
| SLAMF7 | 57823 | IFN-activated macrophages |
| SPP1 | 6696 | IFN-activated macrophages |
| TGM2 | 7052 | IFN-activated macrophages |
| TIMP1 | 7076 | IFN-activated macrophages |
| VCAN | 1462 | IFN-activated macrophages |
| WARS | 7453 | IFN-activated macrophages |
| ABCB10 | 23456 | Phagocytic macrophages |
| ABCC3 | 8714 | Phagocytic macrophages |
| ACAD9 | 28976 | Phagocytic macrophages |
| ACSM5 | 54988 | Phagocytic macrophages |
| ACTL6A | 86 | Phagocytic macrophages |
| ADAM12 | 8038 | Phagocytic macrophages |
| ADORA3 | 140 | Phagocytic macrophages |
| ADRB2 | 154 | Phagocytic macrophages |
| AHNAK2 | 113146 | Phagocytic macrophages |
| AIFM2 | 84883 | Phagocytic macrophages |
| ALS2 | 57679 | Phagocytic macrophages |
| ANKH | 56172 | Phagocytic macrophages |
| AP3M1 | 26985 | Phagocytic macrophages |
| ARMC9 | 80210 | Phagocytic macrophages |
| ARSB | 411 | Phagocytic macrophages |
| ASAP2 | 8853 | Phagocytic macrophages |
| ASB1 | 51665 | Phagocytic macrophages |
| B3GNT7 | 93010 | Phagocytic macrophages |
| BLOC1S5 | 63915 | Phagocytic macrophages |
| BRIX1 | 55299 | Phagocytic macrophages |
| BTD | 686 | Phagocytic macrophages |
| C11orf68 | 83638 | Phagocytic macrophages |
| CALU | 813 | Phagocytic macrophages |
| CCT5 | 22948 | Phagocytic macrophages |
| CD109 | 135228 | Phagocytic macrophages |
| CD276 | 80381 | Phagocytic macrophages |
| CD28 | 940 | Phagocytic macrophages |
| CD3EAP | 10849 | Phagocytic macrophages |
| CD9 | 928 | Phagocytic macrophages |
| CDR2 | 1039 | Phagocytic macrophages |
| CHST13 | 166012 | Phagocytic macrophages |
| CRIP2 | 1397 | Phagocytic macrophages |
| CRYBB1 | 1414 | Phagocytic macrophages |
| CSPG4 | 1464 | Phagocytic macrophages |
| CSTF2T | 23283 | Phagocytic macrophages |
| CTSK | 1513 | Phagocytic macrophages |
| CXADR | 1525 | Phagocytic macrophages |
| DAAM1 | 23002 | Phagocytic macrophages |
| DCBLD2 | 131566 | Phagocytic macrophages |
| DIP2C | 22982 | Phagocytic macrophages |
| DLEU7 | 220107 | Phagocytic macrophages |
| DNM1 | 1759 | Phagocytic macrophages |
| EFNB1 | 1947 | Phagocytic macrophages |
| EMC1 | 23065 | Phagocytic macrophages |
| EMP1 | 2012 | Phagocytic macrophages |
| EPN2 | 22905 | Phagocytic macrophages |
| ERMARD | 55780 | Phagocytic macrophages |
| EXOG | 9941 | Phagocytic macrophages |
| EXOSC8 | 11340 | Phagocytic macrophages |
| EXTL2 | 2135 | Phagocytic macrophages |
| EYA2 | 2139 | Phagocytic macrophages |
| FAIM | 55179 | Phagocytic macrophages |
| FAM134B | 54463 | Phagocytic macrophages |
| FASTKD5 | 60493 | Phagocytic macrophages |
| FBLN5 | 10516 | Phagocytic macrophages |
| FBXO32 | 114907 | Phagocytic macrophages |
| FERMT2 | 10979 | Phagocytic macrophages |
| FGD5 | 152273 | Phagocytic macrophages |
| FUCA1 | 2517 | Phagocytic macrophages |
| FYCO1 | 79443 | Phagocytic macrophages |
| GABRB2 | 2561 | Phagocytic macrophages |
| GATB | 5188 | Phagocytic macrophages |
| GCHFR | 2644 | Phagocytic macrophages |
| GLDN | 342035 | Phagocytic macrophages |
| GNG12 | 55970 | Phagocytic macrophages |
| GNPNAT1 | 64841 | Phagocytic macrophages |
| GOT1 | 2805 | Phagocytic macrophages |
| GPNMB | 10457 | Phagocytic macrophages |
| GPR34 | 2857 | Phagocytic macrophages |
| GSDMA | 284110 | Phagocytic macrophages |
| GSTT1 | 2952 | Phagocytic macrophages |
| HDHD1 | 8226 | Phagocytic macrophages |
| HOXB6 | 3216 | Phagocytic macrophages |
| HPGDS | 27306 | Phagocytic macrophages |
| HTRA1 | 5654 | Phagocytic macrophages |
| IGSF21 | 84966 | Phagocytic macrophages |
| ITGA6 | 3655 | Phagocytic macrophages |
| ITGAE | 3682 | Phagocytic macrophages |
| ITGB5 | 3693 | Phagocytic macrophages |
| KAL1 | 3730 | Phagocytic macrophages |
| KANK1 | 23189 | Phagocytic macrophages |
| KANK2 | 25959 | Phagocytic macrophages |
| KCNQ3 | 3786 | Phagocytic macrophages |
| KCTD10 | 83892 | Phagocytic macrophages |
| KLF9 | 687 | Phagocytic macrophages |
| L1TD1 | 54596 | Phagocytic macrophages |
| LAMC1 | 3915 | Phagocytic macrophages |
| LGI2 | 55203 | Phagocytic macrophages |
| LGMN | 5641 | Phagocytic macrophages |
| LILRB5 | 10990 | Phagocytic macrophages |
| LRRC8A | 56262 | Phagocytic macrophages |
| LZTR1 | 8216 | Phagocytic macrophages |
| MCOLN3 | 55283 | Phagocytic macrophages |
| MERTK | 10461 | Phagocytic macrophages |
| MLF1 | 4291 | Phagocytic macrophages |
| MMD | 23531 | Phagocytic macrophages |
| MMP2 | 4313 | Phagocytic macrophages |
| MRPS14 | 63931 | Phagocytic macrophages |
| MRS2 | 57380 | Phagocytic macrophages |
| MSMO1 | 6307 | Phagocytic macrophages |
| MTUS1 | 57509 | Phagocytic macrophages |
| NAA30 | 122830 | Phagocytic macrophages |
| NAT9 | 26151 | Phagocytic macrophages |
| NFU1 | 27247 | Phagocytic macrophages |
| NRBP2 | 340371 | Phagocytic macrophages |
| NUDCD2 | 134492 | Phagocytic macrophages |
| OAT | 4942 | Phagocytic macrophages |
| OLFML3 | 56944 | Phagocytic macrophages |
| PAQR5 | 54852 | Phagocytic macrophages |
| PAX8 | 7849 | Phagocytic macrophages |
| PDCD11 | 22984 | Phagocytic macrophages |
| PDE1B | 5153 | Phagocytic macrophages |
| PDK4 | 5166 | Phagocytic macrophages |
| PDPN | 10630 | Phagocytic macrophages |
| PGM2 | 55276 | Phagocytic macrophages |
| PGM5 | 5239 | Phagocytic macrophages |
| PIK3CG | 5294 | Phagocytic macrophages |
| PLEK2 | 26499 | Phagocytic macrophages |
| PMP22 | 5376 | Phagocytic macrophages |
| POLM | 27434 | Phagocytic macrophages |
| PPIP5K1 | 9677 | Phagocytic macrophages |
| PPM1N | 147699 | Phagocytic macrophages |
| PRKCDBP | 112464 | Phagocytic macrophages |
| PROS1 | 5627 | Phagocytic macrophages |
| PTGFRN | 5738 | Phagocytic macrophages |
| PTPRM | 5797 | Phagocytic macrophages |
| PTPRN2 | 5799 | Phagocytic macrophages |
| PYCARD-AS1 | 1.01E+08 | Phagocytic macrophages |
| RAB11FIP5 | 26056 | Phagocytic macrophages |
| RAE1 | 8480 | Phagocytic macrophages |
| RBP4 | 5950 | Phagocytic macrophages |
| RGL3 | 57139 | Phagocytic macrophages |
| RIOK1 | 83732 | Phagocytic macrophages |
| RMDN3 | 55177 | Phagocytic macrophages |
| RND3 | 390 | Phagocytic macrophages |
| RPS6KA2 | 6196 | Phagocytic macrophages |
| RRAGA | 10670 | Phagocytic macrophages |
| S1PR1 | 1901 | Phagocytic macrophages |
| SEMA4B | 10509 | Phagocytic macrophages |
| SEPTIN10 | 151011 | Phagocytic macrophages |
| SLC16A6 | 9120 | Phagocytic macrophages |
| SLC1A3 | 6507 | Phagocytic macrophages |
| SLC25A43 | 203427 | Phagocytic macrophages |
| SLC26A11 | 284129 | Phagocytic macrophages |
| SLC29A1 | 2030 | Phagocytic macrophages |
| SLC43A3 | 29015 | Phagocytic macrophages |
| SLC6A12 | 6539 | Phagocytic macrophages |
| SNCA | 6622 | Phagocytic macrophages |
| ST5 | 6764 | Phagocytic macrophages |
| STEAP3 | 55240 | Phagocytic macrophages |
| SYT1 | 6857 | Phagocytic macrophages |
| TBC1D2 | 55357 | Phagocytic macrophages |
| TBCE | 6905 | Phagocytic macrophages |
| TDP2 | 51567 | Phagocytic macrophages |
| TDRD10 | 126668 | Phagocytic macrophages |
| TGFBR1 | 7046 | Phagocytic macrophages |
| THRB | 7068 | Phagocytic macrophages |
| TIMM10 | 26519 | Phagocytic macrophages |
| TMEM106C | 79022 | Phagocytic macrophages |
| TMEM119 | 338773 | Phagocytic macrophages |
| TMEM255B | 348013 | Phagocytic macrophages |
| TNFAIP8L3 | 388121 | Phagocytic macrophages |
| TNFRSF10D | 8793 | Phagocytic macrophages |
| TNFRSF11A | 8792 | Phagocytic macrophages |
| TNS1 | 7145 | Phagocytic macrophages |
| TPPP3 | 51673 | Phagocytic macrophages |
| TREML1 | 340205 | Phagocytic macrophages |
| TRIM21 | 6737 | Phagocytic macrophages |
| TRIM47 | 91107 | Phagocytic macrophages |
| TRIM58 | 25893 | Phagocytic macrophages |
| TUBB2A | 7280 | Phagocytic macrophages |
| TUBB6 | 84617 | Phagocytic macrophages |
| TUBG1 | 7283 | Phagocytic macrophages |
| TXNRD2 | 10587 | Phagocytic macrophages |
| UEVLD | 55293 | Phagocytic macrophages |
| UNC5B | 219699 | Phagocytic macrophages |
| VANGL1 | 81839 | Phagocytic macrophages |
| VPS33A | 65082 | Phagocytic macrophages |
| WDR12 | 55759 | Phagocytic macrophages |
| WDR77 | 79084 | Phagocytic macrophages |
| ZBED3-AS1 | 728723 | Phagocytic macrophages |
| ZBTB7C | 201501 | Phagocytic macrophages |
| ZNF83 | 55769 | Phagocytic macrophages |
| AK3 | 50808 | M1 |
| APOL1 | 8542 | M1 |
| APOL2 | 23780 | M1 |
| APOL3 | 80833 | M1 |
| APOL6 | 80830 | M1 |
| ATF3 | 467 | M1 |
| BCL2A1 | 597 | M1 |
| BIRC3 | 330 | M1 |
| CCL15 | 6359 | M1 |
| CCL19 | 6363 | M1 |
| CCL20 | 6364 | M1 |
| CCL5 | 6352 | M1 |
| CCR7 | 1236 | M1 |
| CHI3L2 | 1117 | M1 |
| CXCL10 | 3627 | M1 |
| CXCL11 | 6373 | M1 |
| CXCL9 | 4283 | M1 |
| EDN1 | 1906 | M1 |
| FAS | 355 | M1 |
| GADD45G | 10912 | M1 |
| HESX1 | 8820 | M1 |
| HSD11B1 | 3290 | M1 |
| IDO1 | 3620 | M1 |
| IGFBP4 | 3487 | M1 |
| IL12B | 3593 | M1 |
| IL15 | 3600 | M1 |
| IL15RA | 3601 | M1 |
| IL2RA | 3559 | M1 |
| IL6 | 3569 | M1 |
| IL7R | 3575 | M1 |
| INHBA | 3624 | M1 |
| IRF1 | 3659 | M1 |
| IRF7 | 3665 | M1 |
| NAMPT | 10135 | M1 |
| OAS2 | 4939 | M1 |
| OASL | 8638 | M1 |
| PDGFA | 5154 | M1 |
| PFKFB3 | 5209 | M1 |
| PFKP | 5214 | M1 |
| PLA1A | 51365 | M1 |
| PSMA2 | 5683 | M1 |
| PSMB9 | 5698 | M1 |
| PSME2 | 5721 | M1 |
| PTX3 | 5806 | M1 |
| SCO2 | 9997 | M1 |
| SLC2A6 | 11182 | M1 |
| SLC31A2 | 1318 | M1 |
| SLC7A5 | 8140 | M1 |
| SLCO5A1 | 81796 | M1 |
| SPHK1 | 8877 | M1 |
| TNF | 7124 | M1 |
| TNFSF10 | 8743 | M1 |
| VCAN | 1462 | M1 |
| XAF1 | 54739 | M1 |
| ADK | 132 | M2 |
| ALOX15 | 246 | M2 |
| CA2 | 760 | M2 |
| CCL13 | 6357 | M2 |
| CCL18 | 6362 | M2 |
| CCL23 | 6368 | M2 |
| CD209 | 30835 | M2 |
| CD36 | 948 | M2 |
| CERK | 64781 | M2 |
| CHN2 | 1124 | M2 |
| CLEC4F | 165530 | M2 |
| CLEC7A | 64581 | M2 |
| CTSC | 1075 | M2 |
| CXCR4 | 7852 | M2 |
| DLC1 | 10395 | M2 |
| EGR2 | 1959 | M2 |
| FGL2 | 10875 | M2 |
| FN1 | 2335 | M2 |
| GAS7 | 8522 | M2 |
| HEXB | 3074 | M2 |
| HNMT | 3176 | M2 |
| HRH1 | 3269 | M2 |
| HS3ST1 | 9957 | M2 |
| HS3ST2 | 9956 | M2 |
| IGF1 | 3479 | M2 |
| LIPA | 3988 | M2 |
| LPAR6 | 10161 | M2 |
| LTA4H | 4048 | M2 |
| MAF | 4094 | M2 |
| MRC1 | 4360 | M2 |
| MS4A4A | 51338 | M2 |
| MS4A6A | 64231 | M2 |
| MSR1 | 4481 | M2 |
| P2RY13 | 53829 | M2 |
| P2RY14 | 9934 | M2 |
| SELENOP | 6414 | M2 |
| SLC38A6 | 145389 | M2 |
| SLC4A7 | 9497 | M2 |
| SLCO2B1 | 11309 | M2 |
| TGFBI | 7045 | M2 |
| TGFBR2 | 7048 | M2 |
| TLR5 | 7100 | M2 |
| TPST2 | 8459 | M2 |
| ARAP2 | 116984 | Martinez S4 |
| ARID5B | 84159 | Martinez S4 |
| ARNTL2 | 56938 | Martinez S4 |
| BATF3 | 55509 | Martinez S4 |
| CCL14 | 6358 | Martinez S4 |
| CCL8 | 6355 | Martinez S4 |
| CD274 | 29126 | Martinez S4 |
| CD38 | 952 | Martinez S4 |
| CFB | 629 | Martinez S4 |
| CKB | 1152 | Martinez S4 |
| CXCL13 | 10563 | Martinez S4 |
| EBI3 | 10148 | Martinez S4 |
| FBX06 | 26270 | Martinez S4 |
| GADD45A | 1647 | Martinez S4 |
| GADD45B | 4616 | Martinez S4 |
| GLS | 2744 | Martinez S4 |
| GOLM1 | 51280 | Martinez S4 |
| GTDC1 | 79712 | Martinez S4 |
| IFI27 | 3429 | Martinez S4 |
| IFITM1 | 8519 | Martinez S4 |
| IL21R | 50615 | Martinez S4 |
| IL32 | 9235 | Martinez S4 |
| JAK3 | 3718 | Martinez S4 |
| KCNJ2 | 3759 | Martinez S4 |
| LHFPL6 | 10186 | Martinez S4 |
| LY75 | 4065 | Martinez S4 |
| MMP12 | 4321 | Martinez S4 |
| MMP25 | 64386 | Martinez S4 |
| MT1M | 4499 | Martinez S4 |
| N4BP1 | 9683 | Martinez S4 |
| NBN | 4683 | Martinez S4 |
| PDCD1LG2 | 80380 | Martinez S4 |
| PXYLP1 | 92370 | Martinez S4 |
| RASGRP1 | 10125 | Martinez S4 |
| SERPINB9 | 5272 | Martinez S4 |
| SERPING1 | 710 | Martinez S4 |
| SLAMF1 | 6504 | Martinez S4 |
| SOCS1 | 8651 | Martinez S4 |
| ST7 | 7982 | Martinez S4 |
| TIFA | 92610 | Martinez S4 |
| TSPAN33 | 340348 | Martinez S4 |
| VCAM1 | 7412 | Martinez S4 |
| ADAMTS14 | 140766 | Martinez S5 |
| CISH | 1154 | Martinez S5 |
| GGT5 | 2687 | Martinez S5 |
| MAG11 | 9223 | Martinez S5 |
| MAOA | 4128 | Martinez S5 |
| STAMBPL1 | 57559 | Martinez S5 |
| SULF2 | 55959 | Martinez S5 |
| TTC9C | 283237 | Martinez S5 |
| CCL11 | 6356 | RnD M1 |
| CCL15 | 6359 | RnD M1 |
| CCL19 | 6363 | RnD M1 |
| CCL2 | 6347 | RnD M1 |
| CCL20 | 6364 | RnD M1 |
| CCL3 | 6348 | RnD M1 |
| CCL4 | 6351 | RnD M1 |
| CCL5 | 6352 | RnD M1 |
| CCL8 | 6355 | RnD M1 |
| CD163 | 9332 | RnD M1 |
| CD36 | 948 | RnD M1 |
| CD68 | 968 | RnD M1 |
| CD80 | 941 | RnD M1 |
| CD86 | 942 | RnD M1 |
| COX2 | 4513 | RnD M1 |
| CX3CL1 | 6376 | RnD M1 |
| CXCL1 | 2919 | RnD M1 |
| CXCL10 | 3627 | RnD M1 |
| CXCL11 | 6373 | RnD M1 |
| CXCL13 | 10563 | RnD M1 |
| CXCL16 | 58191 | RnD M1 |
| CXCL2 | 2920 | RnD M1 |
| CXCL3 | 2921 | RnD M1 |
| CXCL5 | 6374 | RnD M1 |
| CXCL9 | 4283 | RnD M1 |
| FCGR2A | 2212 | RnD M1 |
| FCGR3A | 2214 | RnD M1 |
| HLA-DRA | 3122 | RnD M1 |
| HLA-DRB1 | 3123 | RnD M1 |
| IFNG | 3458 | RnD M1 |
| IFNGR1 | 3459 | RnD M1 |
| IL12A | 3592 | RnD M1 |
| IL12B | 3593 | RnD M1 |
| IL15 | 3600 | RnD M1 |
| IL17A | 3605 | RnD M1 |
| IL18 | 3606 | RnD M1 |
| IL1B | 3553 | RnD M1 |
| IL23A | 51561 | RnD M1 |
| IL6 | 3569 | RnD M1 |
| IL8 | 3576 | RnD M1 |
| IRF5 | 3663 | RnD M1 |
| NOS2 | 4843 | RnD M1 |
| STAT1 | 6772 | RnD M1 |
| TNF | 7124 | RnD M1 |
| ARG1 | 383 | RnD M2a |
| CCL1 | 6346 | RnD M2a |
| CCL14 | 6358 | RnD M2a |
| CCL17 | 6361 | RnD M2a |
| CCL18 | 6362 | RnD M2a |
| CCL2 | 6347 | RnD M2a |
| CCL22 | 6367 | RnD M2a |
| CCL23 | 6368 | RnD M2a |
| CCL24 | 6369 | RnD M2a |
| CCL26 | 10344 | RnD M2a |
| CD163 | 9332 | RnD M2a |
| CD200R1 | 131450 | RnD M2a |
| CD200R1L | 344807 | RnD M2a |
| CD209 | 30835 | RnD M2a |
| CHI3L1 | 1116 | RnD M2a |
| CLEC10A | 10462 | RnD M2a |
| CLEC7A | 64581 | RnD M2a |
| CXCR1 | 3577 | RnD M2a |
| CXCR2 | 3579 | RnD M2a |
| FCER1A | 2205 | RnD M2a |
| HLA-DRA | 3122 | RnD M2a |
| HLA-DRB1 | 3123 | RnD M2a |
| IL10 | 3586 | RnD M2a |
| IL12A | 3592 | RnD M2a |
| IL12B | 3593 | RnD M2a |
| IL1R2 | 7850 | RnD M2a |
| IL1RN | 3557 | RnD M2a |
| IRF4 | 3662 | RnD M2a |
| MRC1 | 4360 | RnD M2a |
| PPARG | 5468 | RnD M2a |
| RETNLB | 84666 | RnD M2a |
| STAT6 | 6778 | RnD M2a |
| TGFB1 | 7040 | RnD M2a |
| CD86 | 942 | RnD M2b |
| CCL1 | 6346 | RnD M2b |
| CCL20 | 6364 | RnD M2b |
| COX2 | 4513 | RnD M2b |
| CXCL1 | 2919 | RnD M2b |
| CXCL2 | 2920 | RnD M2b |
| CXCL3 | 2921 | RnD M2b |
| CSF3 | 1440 | RnD M2b |
| CSF2 | 1437 | RnD M2b |
| IL1B | 3553 | RnD M2b |
| IL4R | 3566 | RnD M2b |
| IL6 | 3569 | RnD M2b |
| IL10 | 3586 | RnD M2b |
| IRF4 | 3662 | RnD M2b |
| HLA-DRA | 3122 | RnD M2b |
| HLA-DRB1 | 3123 | RnD M2b |
| SOCS3 | 9021 | RnD M2b |
| SPHK1 | 8877 | RnD M2b |
| SPHK2 | 56848 | RnD M2b |
| TNF | 7124 | RnD M2b |
| ARG1 | 383 | RnD M2c |
| CCL16 | 6360 | RnD M2c |
| CCL18 | 6362 | RnD M2c |
| CCR2 | 729230 | RnD M2c |
| CD163 | 9332 | RnD M2c |
| CXCL13 | 10563 | RnD M2c |
| IL10 | 3586 | RnD M2c |
| IL4R | 3566 | RnD M2c |
| INHBA | 3624 | RnD M2c |
| IRF4 | 3662 | RnD M2c |
| MRC1 | 4360 | RnD M2c |
| MSR1 | 4481 | RnD M2c |
| SCARB1 | 949 | RnD M2c |
| SLAMF1 | 6504 | RnD M2c |
| SOCS3 | 9021 | RnD M2c |
| TGFB1 | 7040 | RnD M2c |
| TLR1 | 7096 | RnD M2c |
| TLR8 | 51311 | RnD M2c |
| CCL5 | 6352 | RnD M2d |
| CXCL10 | 3627 | RnD M2d |
| CXCL16 | 58191 | RnD M2d |
| IL10 | 3586 | RnD M2d |
| IL12A | 3592 | RnD M2d |
| IL12B | 3593 | RnD M2d |
| NOS2 | 4843 | RnD M2d |
| TNF | 7124 | RnD M2d |
| VEGF | 7422 | RnD M2d |
| APOE | 348 | Reyman alveolar macrophage top 12 |
| CCL2 | 6347 | Reyman alveolar macrophage top 12 |
| CD36 | 948 | Reyman alveolar macrophage top 12 |
| CHI3L1 | 1116 | Reyman alveolar macrophage top 12 |
| FN1 | 2335 | Reyman alveolar macrophage top 12 |
| IL1RN | 3557 | Reyman alveolar macrophage top 12 |
| MARCKS | 4082 | Reyman alveolar macrophage top 12 |
| PLA2G7 | 7941 | Reyman alveolar macrophage top 12 |
| S100A8 | 6279 | Reyman alveolar macrophage top 12 |
| SPP1 | 6696 | Reyman alveolar macrophage top 12 |
| TIMP1 | 7076 | Reyman alveolar macrophage top 12 |
| TREM2 | 54209 | Reyman alveolar macrophage top 12 |
| A2M | 2 | Reyman alveolar macrophage complete UP |
| ABCA1 | 19 | Reyman alveolar macrophage complete UP |
| ABHD2 | 11057 | Reyman alveolar macrophage complete UP |
| ACP2 | 53 | Reyman alveolar macrophage complete UP |
| ACTB | 60 | Reyman alveolar macrophage complete UP |
| ACTG1 | 71 | Reyman alveolar macrophage complete UP |
| ADGRE5 | 976 | Reyman alveolar macrophage complete UP |
| ANXA2 | 302 | Reyman alveolar macrophage complete UP |
| APOC1 | 341 | Reyman alveolar macrophage complete UP |
| APOE | 348 | Reyman alveolar macrophage complete UP |
| ARHGDIB | 397 | Reyman alveolar macrophage complete UP |
| ARID5B | 84159 | Reyman alveolar macrophage complete UP |
| ARPC1B | 10095 | Reyman alveolar macrophage complete UP |
| ATOX1 | 475 | Reyman alveolar macrophage complete UP |
| ATP13A3 | 79572 | Reyman alveolar macrophage complete UP |
| ATP6V1B2 | 526 | Reyman alveolar macrophage complete UP |
| BASP1 | 10409 | Reyman alveolar macrophage complete UP |
| BCAT1 | 586 | Reyman alveolar macrophage complete UP |
| C15orf48 | 84419 | Reyman alveolar macrophage complete UP |
| C6orf62 | 81688 | Reyman alveolar macrophage complete UP |
| CALM3 | 808 | Reyman alveolar macrophage complete UP |
| CALR | 811 | Reyman alveolar macrophage complete UP |
| CAP1 | 10487 | Reyman alveolar macrophage complete UP |
| CCL18 | 6362 | Reyman alveolar macrophage complete UP |
| CCL2 | 6347 | Reyman alveolar macrophage complete UP |
| CCL4 | 6351 | Reyman alveolar macrophage complete UP |
| CD14 | 929 | Reyman alveolar macrophage complete UP |
| CD36 | 948 | Reyman alveolar macrophage complete UP |
| CD4 | 920 | Reyman alveolar macrophage complete UP |
| CD44 | 960 | Reyman alveolar macrophage complete UP |
| CD48 | 962 | Reyman alveolar macrophage complete UP |
| CD84 | 8832 | Reyman alveolar macrophage complete UP |
| CD9 | 928 | Reyman alveolar macrophage complete UP |
| CDC42 | 998 | Reyman alveolar macrophage complete UP |
| CFL1 | 1072 | Reyman alveolar macrophage complete UP |
| CHI3L1 | 1116 | Reyman alveolar macrophage complete UP |
| CHIT1 | 1118 | Reyman alveolar macrophage complete UP |
| CLTC | 1213 | Reyman alveolar macrophage complete UP |
| CMTM3 | 123920 | Reyman alveolar macrophage complete UP |
| CMTM6 | 54918 | Reyman alveolar macrophage complete UP |
| COTL1 | 23406 | Reyman alveolar macrophage complete UP |
| CPM | 1368 | Reyman alveolar macrophage complete UP |
| CRIP1 | 1396 | Reyman alveolar macrophage complete UP |
| CSF1R | 1436 | Reyman alveolar macrophage complete UP |
| CTSB | 1508 | Reyman alveolar macrophage complete UP |
| CTSL | 1514 | Reyman alveolar macrophage complete UP |
| CTSZ | 1522 | Reyman alveolar macrophage complete UP |
| CYBB | 1536 | Reyman alveolar macrophage complete UP |
| CYFIP1 | 23191 | Reyman alveolar macrophage complete UP |
| DPYSL2 | 1808 | Reyman alveolar macrophage complete UP |
| EMP1 | 2012 | Reyman alveolar macrophage complete UP |
| EVL | 51466 | Reyman alveolar macrophage complete UP |
| FCGR2A | 2212 | Reyman alveolar macrophage complete UP |
| FCGR2B | 2213 | Reyman alveolar macrophage complete UP |
| FCGR3A | 2214 | Reyman alveolar macrophage complete UP |
| FGL2 | 10875 | Reyman alveolar macrophage complete UP |
| FKBP1A | 2280 | Reyman alveolar macrophage complete UP |
| FLNA | 2316 | Reyman alveolar macrophage complete UP |
| FN1 | 2335 | Reyman alveolar macrophage complete UP |
| FPR3 | 2359 | Reyman alveolar macrophage complete UP |
| FUCA1 | 2517 | Reyman alveolar macrophage complete UP |
| FXYD5 | 53827 | Reyman alveolar macrophage complete UP |
| FYB | 2533 | Reyman alveolar macrophage complete UP |
| GBP1 | 2633 | Reyman alveolar macrophage complete UP |
| GLIPR1 | 11010 | Reyman alveolar macrophage complete UP |
| GM2A | 2760 | Reyman alveolar macrophage complete UP |
| GSN | 2934 | Reyman alveolar macrophage complete UP |
| HCK | 3055 | Reyman alveolar macrophage complete UP |
| HCST | 10870 | Reyman alveolar macrophage complete UP |
| HIF1A | 3091 | Reyman alveolar macrophage complete UP |
| HLA-A | 3105 | Reyman alveolar macrophage complete UP |
| HLA-DQA2 | 3118 | Reyman alveolar macrophage complete UP |
| HMGN1 | 3150 | Reyman alveolar macrophage complete UP |
| IGF2R | 3482 | Reyman alveolar macrophage complete UP |
| IGHG1 | 3500 | Reyman alveolar macrophage complete UP |
| IGHG3 | 3502 | Reyman alveolar macrophage complete UP |
| IGHG4 | 3503 | Reyman alveolar macrophage complete UP |
| IGKC | 3514 | Reyman alveolar macrophage complete UP |
| IGLC2 | 3538 | Reyman alveolar macrophage complete UP |
| IGSF6 | 10261 | Reyman alveolar macrophage complete UP |
| IL1RN | 3557 | Reyman alveolar macrophage complete UP |
| IL7R | 3575 | Reyman alveolar macrophage complete UP |
| ITGAM | 3684 | Reyman alveolar macrophage complete UP |
| ITGAX | 3687 | Reyman alveolar macrophage complete UP |
| ITGB1 | 3688 | Reyman alveolar macrophage complete UP |
| ITGB2 | 3689 | Reyman alveolar macrophage complete UP |
| KIAA0930 | 23313 | Reyman alveolar macrophage complete UP |
| LAP3 | 51056 | Reyman alveolar macrophage complete UP |
| LASP1 | 3927 | Reyman alveolar macrophage complete UP |
| LCP1 | 3936 | Reyman alveolar macrophage complete UP |
| LDHA | 3939 | Reyman alveolar macrophage complete UP |
| LGMN | 5641 | Reyman alveolar macrophage complete UP |
| LHFPL2 | 10184 | Reyman alveolar macrophage complete UP |
| LILRB4 | 11006 | Reyman alveolar macrophage complete UP |
| LIPA | 3988 | Reyman alveolar macrophage complete UP |
| LITAF | 9516 | Reyman alveolar macrophage complete UP |
| LSP1 | 4046 | Reyman alveolar macrophage complete UP |
| LST1 | 7940 | Reyman alveolar macrophage complete UP |
| MAFB | 9935 | Reyman alveolar macrophage complete UP |
| MARCKS | 4082 | Reyman alveolar macrophage complete UP |
| MNDA | 4332 | Reyman alveolar macrophage complete UP |
| MS4A4A | 51338 | Reyman alveolar macrophage complete UP |
| MS4A6A | 64231 | Reyman alveolar macrophage complete UP |
| MT-ATP6 | 4508 | Reyman alveolar macrophage complete UP |
| MT-ATP8 | 4509 | Reyman alveolar macrophage complete UP |
| MT-CO3 | 4514 | Reyman alveolar macrophage complete UP |
| MT-ND3 | 4537 | Reyman alveolar macrophage complete UP |
| MT-ND6 | 4541 | Reyman alveolar macrophage complete UP |
| NPC2 | 10577 | Reyman alveolar macrophage complete UP |
| NR1H3 | 10062 | Reyman alveolar macrophage complete UP |
| NRP2 | 8828 | Reyman alveolar macrophage complete UP |
| OLR1 | 4973 | Reyman alveolar macrophage complete UP |
| P4HB | 5034 | Reyman alveolar macrophage complete UP |
| PDIA4 | 9601 | Reyman alveolar macrophage complete UP |
| PEA15 | 8682 | Reyman alveolar macrophage complete UP |
| PGK1 | 5230 | Reyman alveolar macrophage complete UP |
| PLA2G7 | 7941 | Reyman alveolar macrophage complete UP |
| PLAC8 | 51316 | Reyman alveolar macrophage complete UP |
| PLEK | 5341 | Reyman alveolar macrophage complete UP |
| PPT1 | 5538 | Reyman alveolar macrophage complete UP |
| PSAP | 5660 | Reyman alveolar macrophage complete UP |
| QSOX1 | 5768 | Reyman alveolar macrophage complete UP |
| RAB31 | 11031 | Reyman alveolar macrophage complete UP |
| RALA | 5898 | Reyman alveolar macrophage complete UP |
| RGS1 | 5996 | Reyman alveolar macrophage complete UP |
| RNASE1 | 6035 | Reyman alveolar macrophage complete UP |
| S100A8 | 6279 | Reyman alveolar macrophage complete UP |
| S100A9 | 6280 | Reyman alveolar macrophage complete UP |
| SAMHD1 | 25939 | Reyman alveolar macrophage complete UP |
| SCGB3A1 | 92304 | Reyman alveolar macrophage complete UP |
| SDC2 | 6383 | Reyman alveolar macrophage complete UP |
| SDS | 10993 | Reyman alveolar macrophage complete UP |
| SEPP1 | 6414 | Reyman alveolar macrophage complete UP |
| SGK1 | 6446 | Reyman alveolar macrophage complete UP |
| SH3BGRL3 | 83442 | Reyman alveolar macrophage complete UP |
| SLA | 6503 | Reyman alveolar macrophage complete UP |
| SLC16A10 | 117247 | Reyman alveolar macrophage complete UP |
| SLC1A3 | 6507 | Reyman alveolar macrophage complete UP |
| SMIM3 | 85027 | Reyman alveolar macrophage complete UP |
| SOD2 | 6648 | Reyman alveolar macrophage complete UP |
| SPARC | 6678 | Reyman alveolar macrophage complete UP |
| SPP1 | 6696 | Reyman alveolar macrophage complete UP |
| SRGN | 5552 | Reyman alveolar macrophage complete UP |
| SRSF2 | 6427 | Reyman alveolar macrophage complete UP |
| STAT1 | 6772 | Reyman alveolar macrophage complete UP |
| TGFBI | 7045 | Reyman alveolar macrophage complete UP |
| TGM2 | 7052 | Reyman alveolar macrophage complete UP |
| TIMP1 | 7076 | Reyman alveolar macrophage complete UP |
| TLN1 | 7094 | Reyman alveolar macrophage complete UP |
| TMEM176B | 28959 | Reyman alveolar macrophage complete UP |
| TMSB10 | 9168 | Reyman alveolar macrophage complete UP |
| TMSB4X | 7114 | Reyman alveolar macrophage complete UP |
| TNFSF13B | 10673 | Reyman alveolar macrophage complete UP |
| TPM3 | 7170 | Reyman alveolar macrophage complete UP |
| TPM4 | 7171 | Reyman alveolar macrophage complete UP |
| TPP1 | 1200 | Reyman alveolar macrophage complete UP |
| TREM2 | 54209 | Reyman alveolar macrophage complete UP |
| TTYH3 | 80727 | Reyman alveolar macrophage complete UP |
| TUBA1B | 10376 | Reyman alveolar macrophage complete UP |
| TYMP | 1890 | Reyman alveolar macrophage complete UP |
| UCP2 | 7351 | Reyman alveolar macrophage complete UP |
| VAMP5 | 10791 | Reyman alveolar macrophage complete UP |
| VMP1 | 81671 | Reyman alveolar macrophage complete UP |
| WARS | 7453 | Reyman alveolar macrophage complete UP |
| WASF2 | 10163 | Reyman alveolar macrophage complete UP |
| ZFP36L1 | 677 | Reyman alveolar macrophage complete UP |

Table of Liao et al Clusters

| GeneSymbol | Cluster | ENTREZID |
|---|---|---|
| S100A9 | G1 | 6280 |
| S100A8 | G1 | 6279 |
| S100A12 | G1 | 6283 |
| VCAN | G1 | 1462 |
| FCN1 | G1 | 2219 |
| CORO1A | G1 | 11151 |
| SELL | G1 | 6402 |
| CD14 | G1 | 929 |
| CFP | G1 | 5199 |
| RNASE2 | G1 | 6036 |
| SERPINB1 | G1 | 1992 |
| FPR1 | G1 | 2357 |
| COTL1 | G1 | 23406 |
| MPEG1 | G1 | 219972 |
| LST1 | G1 | 7940 |
| STAB1 | G1 | 23166 |
| RNASE6 | G1 | 6039 |
| MS4A6A | G1 | 64231 |
| IL1RN | G1 & G2 | 3557 |
| CCL7 | G1 & G2 | 6354 |
| IFITM2 | G1 & G2 | 10581 |
| IFIT2 | G1 & G2 | 3433 |
| PLAC8 | G1 & G2 | 51316 |
| IFIT3 | G1 & G2 | 3437 |
| SERPINB9 | G1 & G2 | 5272 |
| NAMPT | G1 & G2 | 10135 |
| LILRA5 | G1 & G2 | 353514 |
| IER2 | G1 & G2 | 9592 |
| IFITM3 | G1 & G2 | 10410 |
| MX1 | G1 & G2 | 4599 |
| NFKBIA | G1 & G2 | 4792 |
| ANXA2R | G1 & G2 | 389289 |
| RSAD2 | G1 & G2 | 91543 |
| IFIT1 | G1 & G2 | 3434 |
| DUSP6 | G1 & G2 | 1848 |
| ACTB | G1 & G2 | 60 |
| SLC25A37 | G2 | 51312 |
| TNFSF10 | G2 | 8743 |
| CLU | G2 | 1191 |
| CXCL10 | G2 | 3627 |
| APOBEC3A | G2 | 200315 |
| CCL3L1 | G2 | 6349 |
| FFAR2 | G2 | 2867 |
| HSPA6 | G2 | 3310 |
| CCL2 | G2 | 6347 |
| HSPA1A | G2 | 3303 |
| CCL8 | G2 | 6355 |
| DNAJB1 | G2 | 3337 |
| HSPA1B | G2 | 3304 |
| CCL4 | G2 | 6351 |
| CCL3 | G2 | 6348 |
| CCL4L2 | G2 | 9560 |
| HSPB1 | G2 | 3315 |
| IDO1 | G2 | 3620 |
| ISG15 | G2 | 9636 |
| ISG20 | G2 | 3669 |
| TIMP1 | G2 | 7076 |
| NINJ1 | G2 | 4814 |
| VAMP5 | G2 | 10791 |
| CYP1B1 | G2 | 1545 |
| HSPH1 | G2 | 10808 |
| TYMP | G2 | 1890 |
| CXCL11 | G2 | 6373 |
| WARS1 | G2 | 7453 |
| BAG3 | G2 | 9531 |
| GBP1 | G2 | 2633 |
| SOD2 | G2 | 6648 |
| HSP90AA1 | G2 | 3320 |
| BCL2A1 | G2 | 597 |
| GBP5 | G2 | 115362 |
| PLEK | G2 | 5341 |
| NCF1 | G2 | 653361 |
| CALHM6 | G2 | 441168 |
| SLAMF7 | G2 | 57823 |
| SGK1 | G2 | 6446 |
| DEFB1 | G2 | 1672 |
| ANKRD22 | G2 | 118932 |
| GCH1 | G2 | 2643 |
| PLA2G7 | G2 | 7941 |
| CTSB | G2 | 1508 |
| HAMP | G3 | 57817 |
| LGMN | G3 | 5641 |
| RGS1 | G3 | 5996 |
| SPP1 | G3 | 6696 |
| RNASE1 | G3 | 6035 |
| HMOX1 | G3 | 3162 |
| GPR183 | G3 | 1880 |
| ARL4C | G3 | 10123 |
| C1QC | G3 | 714 |
| SDS | G3 | 10993 |
| TGFB1 | G3 | 7040 |
| A2M | G3 | 2 |
| FPR3 | G3 | 2359 |
| CD84 | G3 | 8832 |
| NRP2 | G3 | 8828 |
| CREG1 | G3 | 8804 |
| SDC3 | G3 | 9672 |
| CTSL | G3 | 1514 |
| MMP14 | G3 | 4323 |
| SMPDL3A | G3 | 10924 |
| PMP22 | G3 | 5376 |
| PLD3 | G3 | 23646 |
| LIPA | G3 | 3988 |
| MS4A4A | G3 | 51338 |
| CSF1R | G3 | 1436 |
| CD86 | G3 | 942 |
| GPNMB | G3 | 10457 |
| CREM | G3 | 1390 |
| APOE | G3 | 348 |
| HSP90B1 | G3 | 7184 |
| C1QB | G3 | 713 |
| CCL18 | G3 | 6362 |
| IL7R | G3 | 3575 |
| CXCL8 | G3 | 3576 |
| IFITM1 | G3 | 8519 |
| FABP5 | G4 | 2171 |
| FABP4 | G4 | 2167 |
| NUPR1 | G4 | 26471 |
| APOC1 | G4 | 341 |
| GCHFR | G4 | 2644 |
| INHBA | G4 | 3624 |
| ALOX5AP | G4 | 241 |
| ALDH2 | G4 | 217 |
| CD52 | G4 | 1043 |
| HLA-DQA1 | G4 | 3117 |
| HLA-DQB1 | G4 | 3119 |
| LPL | G4 | 4023 |
| MCEMP1 | G4 | 199675 |
| MARCO | G4 | 8685 |
| TFRC | G4 | 7037 |
| HPGD | G4 | 3248 |
| FBP1 | G4 | 2203 |
| RBP3 | G4 | 5949 |
| ACP5 | G4 | 54 |
| PEBP1 | G4 | 5037 |
| MSR1 | G4 | 4481 |
| MRC1 | G4 | 4360 |
| AKR1C3 | G4 | 8644 |

Table of PPI Putative Myeloid Population

| geneSymbol | EntrezID | Category |
|---|---|---|
| PLD3 | 23646 | PPI PBMC Myeloid Subcluster 1 |
| NMNAT1 | 64802 | PPI PBMC Myeloid Subcluster 1 |
| CSGALNACT2 | 55454 | PPI PBMC Myeloid Subcluster 1 |
| DSE | 29940 | PPI PBMC Myeloid Subcluster 1 |
| MLEC | 9761 | PPI PBMC Myeloid Subcluster 1 |
| DNAJC5 | 80331 | PPI PBMC Myeloid Subcluster 1 |
| ALDH3B1 | 221 | PPI PBMC Myeloid Subcluster 1 |
| AGPAT2 | 10555 | PPI PBMC Myeloid Subcluster 1 |
| QSOX1 | 5768 | PPI PBMC Myeloid Subcluster 1 |
| SLC2A5 | 6518 | PPI PBMC Myeloid Subcluster 1 |
| MFGE8 | 4240 | PPI PBMC Myeloid Subcluster 1 |
| BST1 | 683 | PPI PBMC Myeloid Subcluster 1 |
| VAMP8 | 8673 | PPI PBMC Myeloid Subcluster 1 |
| HVCN1 | 84329 | PPI PBMC Myeloid Subcluster 1 |
| ATP11A | 23250 | PPI PBMC Myeloid Subcluster 1 |
| IGFBP7 | 3490 | PPI PBMC Myeloid Subcluster 1 |
| F5 | 2153 | PPI PBMC Myeloid Subcluster 1 |
| VCAN | 1462 | PPI PBMC Myeloid Subcluster 1 |
| CD93 | 22918 | PPI PBMC Myeloid Subcluster 1 |
| LGALS1 | 3956 | PPI PBMC Myeloid Subcluster 1 |
| TIMP1 | 7076 | PPI PBMC Myeloid Subcluster 1 |
| RAB44 | 401258 | PPI PBMC Myeloid Subcluster 1 |
| CLEC4D | 338339 | PPI PBMC Myeloid Subcluster 1 |
| MERTK | 10461 | PPI PBMC Myeloid Subcluster 1 |
| GAS6 | 2621 | PPI PBMC Myeloid Subcluster 1 |
| CLEC5A | 23601 | PPI PBMC Myeloid Subcluster 1 |
| LRP8 | 7804 | PPI PBMC Myeloid Subcluster 1 |
| MCEMP1 | 199675 | PPI PBMC Myeloid Subcluster 1 |
| CLEC12A | 160364 | PPI PBMC Myeloid Subcluster 1 |
| APOE | 348 | PPI PBMC Myeloid Subcluster 1 |
| CD36 | 948 | PPI PBMC Myeloid Subcluster 1 |
| PLD1 | 5337 | PPI PBMC Myeloid Subcluster 1 |
| CD33 | 945 | PPI PBMC Myeloid Subcluster 1 |
| LAIR1 | 3903 | PPI PBMC Myeloid Subcluster 1 |
| FSTL3 | 10272 | PPI PBMC Myeloid Subcluster 1 |
| BACE1 | 23621 | PPI PBMC Myeloid Subcluster 1 |
| CST3 | 1471 | PPI PBMC Myeloid Subcluster 1 |
| CYBB | 1536 | PPI PBMC Myeloid Subcluster 1 |
| NUCB1 | 4924 | PPI PBMC Myeloid Subcluster 1 |
| C3AR1 | 719 | PPI PBMC Myeloid Subcluster 1 |
| APLP2 | 334 | PPI PBMC Myeloid Subcluster 1 |
| APP | 351 | PPI PBMC Myeloid Subcluster 1 |
| ITGB2 | 3689 | PPI PBMC Myeloid Subcluster 1 |
| ITGAM | 3684 | PPI PBMC Myeloid Subcluster 1 |
| LAMC1 | 3915 | PPI PBMC Myeloid Subcluster 1 |
| LAMB2 | 3913 | PPI PBMC Myeloid Subcluster 1 |
| PCSK9 | 255738 | PPI PBMC Myeloid Subcluster 1 |
| FN1 | 2335 | PPI PBMC Myeloid Subcluster 1 |
| FAM20C | 56975 | PPI PBMC Myeloid Subcluster 1 |
| FAM20A | 54757 | PPI PBMC Myeloid Subcluster 1 |
| CALU | 813 | PPI PBMC Myeloid Subcluster 1 |
| P4HB | 5034 | PPI PBMC Myeloid Subcluster 1 |
| PDIA6 | 10130 | PPI PBMC Myeloid Subcluster 1 |
| ATP6V1D | 51382 | PPI PBMC Myeloid Subcluster 1 |
| LAMTOR2 | 28956 | PPI PBMC Myeloid Subcluster 1 |
| VPS11 | 55823 | PPI PBMC Myeloid Subcluster 3 |
| UBE2L3 | 7332 | PPI PBMC Myeloid Subcluster 3 |
| GPER1 | 2852 | PPI PBMC Myeloid Subcluster 3 |
| S1PR3 | 1903 | PPI PBMC Myeloid Subcluster 3 |
| MT2A | 4502 | PPI PBMC Myeloid Subcluster 3 |
| HEBP1 | 50865 | PPI PBMC Myeloid Subcluster 3 |
| SAMHD1 | 25939 | PPI PBMC Myeloid Subcluster 3 |
| OPN3 | 23596 | PPI PBMC Myeloid Subcluster 3 |
| GNG5 | 2787 | PPI PBMC Myeloid Subcluster 3 |
| CXCL10 | 3627 | PPI PBMC Myeloid Subcluster 3 |
| TBC1D2 | 55357 | PPI PBMC Myeloid Subcluster 3 |
| IRF5 | 3663 | PPI PBMC Myeloid Subcluster 3 |
| ADORA3 | 140 | PPI PBMC Myeloid Subcluster 3 |
| CCR2 | 729230 | PPI PBMC Myeloid Subcluster 3 |
| IFITM3 | 10410 | PPI PBMC Myeloid Subcluster 3 |
| FPR3 | 2359 | PPI PBMC Myeloid Subcluster 3 |
| IRF8 | 3394 | PPI PBMC Myeloid Subcluster 3 |
| RAB7A | 7879 | PPI PBMC Myeloid Subcluster 3 |
| CCZ1 | 51622 | PPI PBMC Myeloid Subcluster 3 |
| CCR1 | 1230 | PPI PBMC Myeloid Subcluster 3 |
| IFI35 | 3430 | PPI PBMC Myeloid Subcluster 3 |
| FCGR1B | 2210 | PPI PBMC Myeloid Subcluster 3 |
| IFI6 | 2537 | PPI PBMC Myeloid Subcluster 3 |
| RSAD2 | 91543 | PPI PBMC Myeloid Subcluster 3 |
| MX1 | 4599 | PPI PBMC Myeloid Subcluster 3 |
| GALK2 | 2585 | PPI PBMC Myeloid Subcluster 4 |
| CTSB | 1508 | PPI PBMC Myeloid Subcluster 4 |
| CTSL | 1514 | PPI PBMC Myeloid Subcluster 4 |
| SGMS2 | 166929 | PPI PBMC Myeloid Subcluster 4 |
| METTL7A | 25840 | PPI PBMC Myeloid Subcluster 4 |
| ASAH1 | 427 | PPI PBMC Myeloid Subcluster 4 |
| MMP17 | 4326 | PPI PBMC Myeloid Subcluster 4 |
| CFP | 5199 | PPI PBMC Myeloid Subcluster 4 |
| CTSS | 1520 | PPI PBMC Myeloid Subcluster 4 |
| NIPA2 | 81614 | PPI PBMC Myeloid Subcluster 4 |
| C5 | 727 | PPI PBMC Myeloid Subcluster 4 |
| IDH1 | 3417 | PPI PBMC Myeloid Subcluster 4 |
| LTA4H | 4048 | PPI PBMC Myeloid Subcluster 4 |
| ALDOA | 226 | PPI PBMC Myeloid Subcluster 4 |
| CTSD | 1509 | PPI PBMC Myeloid Subcluster 4 |
| HP | 3240 | PPI PBMC Myeloid Subcluster 4 |
| CTSH | 1512 | PPI PBMC Myeloid Subcluster 4 |
| CSTB | 1476 | PPI PBMC Myeloid Subcluster 4 |
| TIMP2 | 7077 | PPI PBMC Myeloid Subcluster 4 |
| MMP14 | 4323 | PPI PBMC Myeloid Subcluster 4 |
| HLA-DQA1 | 3117 | PPI PBMC Myeloid Subcluster 4 |
| HLA-DRA | 3122 | PPI PBMC Myeloid Subcluster 4 |
| FCGR1A | 2209 | PPI PBMC Myeloid Subcluster 4 |
| CYFIP1 | 23191 | PPI PBMC Myeloid Subcluster 4 |
| NACA2 | 342538 | PPI PBMC Myeloid Subcluster 5 |
| NCAPH | 23397 | PPI PBMC Myeloid Subcluster 5 |
| PER1 | 5187 | PPI PBMC Myeloid Subcluster 5 |
| PPP1R3D | 5509 | PPI PBMC Myeloid Subcluster 5 |
| TP53BP2 | 7159 | PPI PBMC Myeloid Subcluster 5 |
| CMPK2 | 129607 | PPI PBMC Myeloid Subcluster 5 |
| RCC2 | 55920 | PPI PBMC Myeloid Subcluster 5 |
| MAPRE1 | 22919 | PPI PBMC Myeloid Subcluster 5 |
| NUP37 | 79023 | PPI PBMC Myeloid Subcluster 5 |
| POLQ | 10721 | PPI PBMC Myeloid Subcluster 5 |
| PPP1CC | 5501 | PPI PBMC Myeloid Subcluster 5 |
| OAS2 | 4939 | PPI PBMC Myeloid Subcluster 5 |
| OAS3 | 4940 | PPI PBMC Myeloid Subcluster 5 |
| OAS1 | 4938 | PPI PBMC Myeloid Subcluster 5 |
| OASL | 8638 | PPI PBMC Myeloid Subcluster 5 |
| FEN1 | 2237 | PPI PBMC Myeloid Subcluster 5 |
| PCNA | 5111 | PPI PBMC Myeloid Subcluster 5 |
| CDT1 | 81620 | PPI PBMC Myeloid Subcluster 5 |
| MCM2 | 4171 | PPI PBMC Myeloid Subcluster 5 |
| CDC6 | 990 | PPI PBMC Myeloid Subcluster 5 |
| ORC1 | 4998 | PPI PBMC Myeloid Subcluster 5 |
| MCM4 | 4173 | PPI PBMC Myeloid Subcluster 5 |
| MARCO | 8685 | PPI PBMC Myeloid Subcluster 6 |
| CCDC47 | 57003 | PPI PBMC Myeloid Subcluster 6 |
| MPEG1 | 219972 | PPI PBMC Myeloid Subcluster 6 |
| ALOX5 | 240 | PPI PBMC Myeloid Subcluster 6 |
| TLR5 | 7100 | PPI PBMC Myeloid Subcluster 6 |
| IKBKE | 9641 | PPI PBMC Myeloid Subcluster 6 |
| MRC1 | 4360 | PPI PBMC Myeloid Subcluster 6 |
| C2 | 717 | PPI PBMC Myeloid Subcluster 6 |
| TLR2 | 7097 | PPI PBMC Myeloid Subcluster 6 |
| CD14 | 929 | PPI PBMC Myeloid Subcluster 6 |
| IRAK3 | 11213 | PPI PBMC Myeloid Subcluster 6 |
| IRAK1 | 3654 | PPI PBMC Myeloid Subcluster 6 |
| S100A12 | 6283 | PPI PBMC Myeloid Subcluster 6 |
| TLR4 | 7099 | PPI PBMC Myeloid Subcluster 6 |
| MYD88 | 4615 | PPI PBMC Myeloid Subcluster 6 |
| CD180 | 4064 | PPI PBMC Myeloid Subcluster 6 |
| LY86 | 9450 | PPI PBMC Myeloid Subcluster 6 |
| S100A8 | 6279 | PPI PBMC Myeloid Subcluster 6 |
| S100A9 | 6280 | PPI PBMC Myeloid Subcluster 6 |
| C1QC | 714 | PPI PBMC Myeloid Subcluster 6 |
| C1QB | 713 | PPI PBMC Myeloid Subcluster 6 |
| C1QA | 712 | PPI PBMC Myeloid Subcluster 6 |
| MLKL | 197259 | PPI PBMC Myeloid Subcluster 13 |
| PTBP2 | 58155 | PPI PBMC Myeloid Subcluster 13 |
| NLRP3 | 114548 | PPI PBMC Myeloid Subcluster 13 |
| CARD16 | 114769 | PPI PBMC Myeloid Subcluster 13 |
| IL18 | 3606 | PPI PBMC Myeloid Subcluster 13 |
| CASP1 | 834 | PPI PBMC Myeloid Subcluster 13 |
| NLRC4 | 58484 | PPI PBMC Myeloid Subcluster 13 |
| NAIP | 4671 | PPI PBMC Myeloid Subcluster 13 |
| ADRA2A | 150 | PPI Lung Myeloid Subcluster 1 |
| BCL3 | 602 | PPI Lung Myeloid Subcluster 1 |
| C3AR1 | 719 | PPI Lung Myeloid Subcluster 1 |
| C5AR1 | 728 | PPI Lung Myeloid Subcluster 1 |
| CCL15 | 6359 | PPI Lung Myeloid Subcluster 1 |
| CCL19 | 6363 | PPI Lung Myeloid Subcluster 1 |
| CCL3 | 6348 | PPI Lung Myeloid Subcluster 1 |
| CCL4 | 6351 | PPI Lung Myeloid Subcluster 1 |
| CCR1 | 1230 | PPI Lung Myeloid Subcluster 1 |
| CCRL2 | 9034 | PPI Lung Myeloid Subcluster 1 |
| CMPK2 | 129607 | PPI Lung Myeloid Subcluster 1 |
| CXCL10 | 3627 | PPI Lung Myeloid Subcluster 1 |
| CXCL11 | 6373 | PPI Lung Myeloid Subcluster 1 |
| CXCL16 | 58191 | PPI Lung Myeloid Subcluster 1 |
| CXCR2 | 3579 | PPI Lung Myeloid Subcluster 1 |
| DDX58 | 23586 | PPI Lung Myeloid Subcluster 1 |
| DDX60 | 55601 | PPI Lung Myeloid Subcluster 1 |
| DDX60L | 91351 | PPI Lung Myeloid Subcluster 1 |
| DRD2 | 1813 | PPI Lung Myeloid Subcluster 1 |
| EIF2AK2 | 5610 | PPI Lung Myeloid Subcluster 1 |
| EPSTI1 | 94240 | PPI Lung Myeloid Subcluster 1 |
| FPR1 | 2357 | PPI Lung Myeloid Subcluster 1 |
| FPR2 | 2358 | PPI Lung Myeloid Subcluster 1 |
| GNG2 | 54331 | PPI Lung Myeloid Subcluster 1 |
| GNG5 | 2787 | PPI Lung Myeloid Subcluster 1 |
| GPR37L1 | 9283 | PPI Lung Myeloid Subcluster 1 |
| GPSM3 | 63940 | PPI Lung Myeloid Subcluster 1 |
| HCAR1 | 27198 | PPI Lung Myeloid Subcluster 1 |
| HCAR2 | 338442 | PPI Lung Myeloid Subcluster 1 |
| HELZ2 | 85441 | PPI Lung Myeloid Subcluster 1 |
| HERC5 | 51191 | PPI Lung Myeloid Subcluster 1 |
| HERC6 | 55008 | PPI Lung Myeloid Subcluster 1 |
| HLA-A | 3105 | PPI Lung Myeloid Subcluster 1 |
| HLA-B | 3106 | PPI Lung Myeloid Subcluster 1 |
| HTR1A | 3350 | PPI Lung Myeloid Subcluster 1 |
| IDO1 | 3620 | PPI Lung Myeloid Subcluster 1 |
| IFI44 | 10561 | PPI Lung Myeloid Subcluster 1 |
| IFI44L | 10964 | PPI Lung Myeloid Subcluster 1 |
| IFI6 | 2537 | PPI Lung Myeloid Subcluster 1 |
| IFIH1 | 64135 | PPI Lung Myeloid Subcluster 1 |
| IFIT1 | 3434 | PPI Lung Myeloid Subcluster 1 |
| IFIT1B | 439996 | PPI Lung Myeloid Subcluster 1 |
| IFIT2 | 3433 | PPI Lung Myeloid Subcluster 1 |
| IFIT3 | 3437 | PPI Lung Myeloid Subcluster 1 |
| IFIT5 | 24138 | PPI Lung Myeloid Subcluster 1 |
| IFITM1 | 8519 | PPI Lung Myeloid Subcluster 1 |
| IFITM2 | 10581 | PPI Lung Myeloid Subcluster 1 |
| IFITM3 | 10410 | PPI Lung Myeloid Subcluster 1 |
| IL27 | 246778 | PPI Lung Myeloid Subcluster 1 |
| IRF2 | 3660 | PPI Lung Myeloid Subcluster 1 |
| IRF7 | 3665 | PPI Lung Myeloid Subcluster 1 |
| ISG15 | 9636 | PPI Lung Myeloid Subcluster 1 |
| KCNJ2 | 3759 | PPI Lung Myeloid Subcluster 1 |
| MX1 | 4599 | PPI Lung Myeloid Subcluster 1 |
| MX2 | 4600 | PPI Lung Myeloid Subcluster 1 |
| OAS1 | 4938 | PPI Lung Myeloid Subcluster 1 |
| OAS2 | 4939 | PPI Lung Myeloid Subcluster 1 |
| OAS3 | 4940 | PPI Lung Myeloid Subcluster 1 |
| OASL | 8638 | PPI Lung Myeloid Subcluster 1 |
| P2RY13 | 53829 | PPI Lung Myeloid Subcluster 1 |
| P2RY14 | 9934 | PPI Lung Myeloid Subcluster 1 |
| RSAD2 | 91543 | PPI Lung Myeloid Subcluster 1 |
| RTP4 | 64108 | PPI Lung Myeloid Subcluster 1 |
| SAA1 | 6288 | PPI Lung Myeloid Subcluster 1 |
| SAMD9 | 54809 | PPI Lung Myeloid Subcluster 1 |
| SAMD9L | 219285 | PPI Lung Myeloid Subcluster 1 |
| SP110 | 3431 | PPI Lung Myeloid Subcluster 1 |
| STAT1 | 6772 | PPI Lung Myeloid Subcluster 1 |
| STAT2 | 6773 | PPI Lung Myeloid Subcluster 1 |
| SUCNR1 | 56670 | PPI Lung Myeloid Subcluster 1 |
| TNFSF10 | 8743 | PPI Lung Myeloid Subcluster 1 |
| UBE2L6 | 9246 | PPI Lung Myeloid Subcluster 1 |
| USP15 | 9958 | PPI Lung Myeloid Subcluster 1 |
| USP18 | 11274 | PPI Lung Myeloid Subcluster 1 |
| XAF1 | 54739 | PPI Lung Myeloid Subcluster 1 |
| ZBP1 | 81030 | PPI Lung Myeloid Subcluster 1 |
| CCL2 | 6347 | PPI Lung Myeloid Subcluster 2 |
| CCL7 | 6354 | PPI Lung Myeloid Subcluster 2 |
| CDA | 978 | PPI Lung Myeloid Subcluster 2 |
| CHI3L1 | 1116 | PPI Lung Myeloid Subcluster 2 |
| CHIT1 | 1118 | PPI Lung Myeloid Subcluster 2 |
| CRISP3 | 10321 | PPI Lung Myeloid Subcluster 2 |
| FLG2 | 388698 | PPI Lung Myeloid Subcluster 2 |
| HBB | 3043 | PPI Lung Myeloid Subcluster 2 |
| HP | 3240 | PPI Lung Myeloid Subcluster 2 |
| LCN2 | 3934 | PPI Lung Myeloid Subcluster 2 |
| LTF | 4057 | PPI Lung Myeloid Subcluster 2 |
| MMP8 | 4317 | PPI Lung Myeloid Subcluster 2 |
| PGLYRP1 | 8993 | PPI Lung Myeloid Subcluster 2 |
| TCN1 | 6947 | PPI Lung Myeloid Subcluster 2 |
| MAP2K6 | 5608 | PPI Lung Myeloid Subcluster 8 |
| MCTS1 | 28985 | PPI Lung Myeloid Subcluster 8 |
| PSMA4 | 5685 | PPI Lung Myeloid Subcluster 8 |
| PSME3 | 10197 | PPI Lung Myeloid Subcluster 8 |
| TNF | 7124 | PPI Lung Myeloid Subcluster 8 |
| TNFSF13B | 10673 | PPI Lung Myeloid Subcluster 8 |
| TNFSF14 | 8740 | PPI Lung Myeloid Subcluster 8 |
| TP73 | 7161 | PPI Lung Myeloid Subcluster 8 |
| AIM2 | 9447 | PPI Lung Myeloid Subcluster 9 |
| CASP4 | 837 | PPI Lung Myeloid Subcluster 9 |
| CASP5 | 838 | PPI Lung Myeloid Subcluster 9 |
| CASP1 | 834 | PPI Lung Myeloid Subcluster 10 |
| IL1A | 3552 | PPI Lung Myeloid Subcluster 10 |
| IL1R2 | 7850 | PPI Lung Myeloid Subcluster 10 |
| IL1RN | 3557 | PPI Lung Myeloid Subcluster 10 |
| ADCYAP1 | 116 | PPI Lung Myeloid Subcluster 3 |
| BST1 | 683 | PPI Lung Myeloid Subcluster 3 |
| CD38 | 952 | PPI Lung Myeloid Subcluster 3 |
| CD53 | 963 | PPI Lung Myeloid Subcluster 3 |
| CEACAM1 | 634 | PPI Lung Myeloid Subcluster 3 |
| CERS6 | 253782 | PPI Lung Myeloid Subcluster 3 |
| CLEC12A | 160364 | PPI Lung Myeloid Subcluster 3 |
| CLEC12B | 387837 | PPI Lung Myeloid Subcluster 3 |
| CLEC4D | 338339 | PPI Lung Myeloid Subcluster 3 |
| CLEC4E | 26253 | PPI Lung Myeloid Subcluster 3 |
| CYBB | 1536 | PPI Lung Myeloid Subcluster 3 |
| CYSTM1 | 84418 | PPI Lung Myeloid Subcluster 3 |
| FCER1G | 2207 | PPI Lung Myeloid Subcluster 3 |
| GPR84 | 53831 | PPI Lung Myeloid Subcluster 3 |
| MGAM | 8972 | PPI Lung Myeloid Subcluster 3 |
| MS4A3 | 932 | PPI Lung Myeloid Subcluster 3 |
| ALOX5AP | 241 | PPI Lung Myeloid Subcluster 6 |
| AQP9 | 366 | PPI Lung Myeloid Subcluster 6 |
| ARHGAP9 | 64333 | PPI Lung Myeloid Subcluster 6 |
| C1QB | 713 | PPI Lung Myeloid Subcluster 6 |
| C2 | 717 | PPI Lung Myeloid Subcluster 6 |
| CAPZA1 | 829 | PPI Lung Myeloid Subcluster 6 |
| CCL18 | 6362 | PPI Lung Myeloid Subcluster 6 |
| CCL8 | 6355 | PPI Lung Myeloid Subcluster 6 |
| CD74 | 972 | PPI Lung Myeloid Subcluster 6 |
| CMTM2 | 146225 | PPI Lung Myeloid Subcluster 6 |
| COL9A2 | 1298 | PPI Lung Myeloid Subcluster 6 |
| CTF1 | 1489 | PPI Lung Myeloid Subcluster 6 |
| EVI2A | 2123 | PPI Lung Myeloid Subcluster 6 |
| EVI2B | 2124 | PPI Lung Myeloid Subcluster 6 |
| FCGR2A | 2212 | PPI Lung Myeloid Subcluster 6 |
| FCN1 | 2219 | PPI Lung Myeloid Subcluster 6 |
| GAPT | 202309 | PPI Lung Myeloid Subcluster 6 |
| HCST | 10870 | PPI Lung Myeloid Subcluster 6 |
| IFNL1 | 282618 | PPI Lung Myeloid Subcluster 6 |
| IGLL5 | 100423062 | PPI Lung Myeloid Subcluster 6 |
| IGSF6 | 10261 | PPI Lung Myeloid Subcluster 6 |
| IL2RG | 3561 | PPI Lung Myeloid Subcluster 6 |
| LILRA1 | 11024 | PPI Lung Myeloid Subcluster 6 |
| LY86 | 9450 | PPI Lung Myeloid Subcluster 6 |
| NCF4 | 4689 | PPI Lung Myeloid Subcluster 6 |
| PIK3AP1 | 118788 | PPI Lung Myeloid Subcluster 6 |
| PLEK | 5341 | PPI Lung Myeloid Subcluster 6 |
| RAC2 | 5880 | PPI Lung Myeloid Subcluster 6 |
| RNASE2 | 6036 | PPI Lung Myeloid Subcluster 6 |
| S100A12 | 6283 | PPI Lung Myeloid Subcluster 6 |
| S100A8 | 6279 | PPI Lung Myeloid Subcluster 6 |
| S100A9 | 6280 | PPI Lung Myeloid Subcluster 6 |
| SELL | 6402 | PPI Lung Myeloid Subcluster 6 |
| STX11 | 8676 | PPI Lung Myeloid Subcluster 6 |
| TAGAP | 117289 | PPI Lung Myeloid Subcluster 6 |
| TFEC | 22797 | PPI Lung Myeloid Subcluster 6 |
| TLR1 | 7096 | PPI Lung Myeloid Subcluster 6 |
| TMEM176B | 28959 | PPI Lung Myeloid Subcluster 6 |
| BCL2A1 | 597 | PPI Lung Myeloid Subcluster 4 |
| C1QC | 714 | PPI Lung Myeloid Subcluster 4 |
| CARD16 | 114769 | PPI Lung Myeloid Subcluster 4 |
| CLEC4A | 50856 | PPI Lung Myeloid Subcluster 4 |
| CSF2RB | 1439 | PPI Lung Myeloid Subcluster 4 |
| FGL2 | 10875 | PPI Lung Myeloid Subcluster 4 |
| IL1B | 3553 | PPI Lung Myeloid Subcluster 4 |
| RGS18 | 64407 | PPI Lung Myeloid Subcluster 4 |
| TLR7 | 51284 | PPI Lung Myeloid Subcluster 4 |
| ARRB2 | 409 | PPI Lung Myeloid Subcluster 7 |
| C6 | 729 | PPI Lung Myeloid Subcluster 7 |
| C8A | 731 | PPI Lung Myeloid Subcluster 7 |
| CDH2 | 1000 | PPI Lung Myeloid Subcluster 7 |
| EGF | 1950 | PPI Lung Myeloid Subcluster 7 |
| F5 | 2153 | PPI Lung Myeloid Subcluster 7 |
| FGG | 2266 | PPI Lung Myeloid Subcluster 7 |
| FGL1 | 2267 | PPI Lung Myeloid Subcluster 7 |
| IGFBP1 | 3484 | PPI Lung Myeloid Subcluster 7 |
| LMAN1L | 79748 | PPI Lung Myeloid Subcluster 7 |
| LY96 | 23643 | PPI Lung Myeloid Subcluster 7 |
| LYN | 4067 | PPI Lung Myeloid Subcluster 7 |
| OR13G1 | 441933 | PPI Lung Myeloid Subcluster 7 |
| OR2AT4 | 341152 | PPI Lung Myeloid Subcluster 7 |
| OR2C1 | 4993 | PPI Lung Myeloid Subcluster 7 |
| PCSK9 | 255738 | PPI Lung Myeloid Subcluster 7 |
| RBP4 | 5950 | PPI Lung Myeloid Subcluster 7 |
| SERPINA1 | 5265 | PPI Lung Myeloid Subcluster 7 |
| SERPINE2 | 5270 | PPI Lung Myeloid Subcluster 7 |
| BDKRB2 | 624 | PPI BAL Myeloid Subcluster 2 |
| C3 | 718 | PPI BAL Myeloid Subcluster 2 |
| C3AR1 | 719 | PPI BAL Myeloid Subcluster 2 |
| CCL20 | 6364 | PPI BAL Myeloid Subcluster 2 |
| CCL4 | 6351 | PPI BAL Myeloid Subcluster 2 |
| CCL7 | 6354 | PPI BAL Myeloid Subcluster 2 |
| CCR1 | 1230 | PPI BAL Myeloid Subcluster 2 |
| CCR5 | 1234 | PPI BAL Myeloid Subcluster 2 |
| CXCL1 | 2919 | PPI BAL Myeloid Subcluster 2 |
| CXCL10 | 3627 | PPI BAL Myeloid Subcluster 2 |
| CXCL11 | 6373 | PPI BAL Myeloid Subcluster 2 |
| CXCL12 | 6387 | PPI BAL Myeloid Subcluster 2 |
| CXCL16 | 58191 | PPI BAL Myeloid Subcluster 2 |
| CXCL2 | 2920 | PPI BAL Myeloid Subcluster 2 |
| CXCL3 | 2921 | PPI BAL Myeloid Subcluster 2 |
| CXCL6 | 6372 | PPI BAL Myeloid Subcluster 2 |
| CXCL9 | 4283 | PPI BAL Myeloid Subcluster 2 |
| FPR3 | 2359 | PPI BAL Myeloid Subcluster 2 |
| GNAI1 | 2770 | PPI BAL Myeloid Subcluster 2 |
| GNB4 | 59345 | PPI BAL Myeloid Subcluster 2 |
| GNG12 | 55970 | PPI BAL Myeloid Subcluster 2 |
| GNG5 | 2787 | PPI BAL Myeloid Subcluster 2 |
| GNGT2 | 2793 | PPI BAL Myeloid Subcluster 2 |
| HCAR2 | 338442 | PPI BAL Myeloid Subcluster 2 |
| KCNJ10 | 3766 | PPI BAL Myeloid Subcluster 2 |
| KCNJ16 | 3773 | PPI BAL Myeloid Subcluster 2 |
| KCNJ5 | 3762 | PPI BAL Myeloid Subcluster 2 |
| LPAR1 | 1902 | PPI BAL Myeloid Subcluster 2 |
| LPAR3 | 23566 | PPI BAL Myeloid Subcluster 2 |
| NPW | 283869 | PPI BAL Myeloid Subcluster 2 |
| PAQR5 | 54852 | PPI BAL Myeloid Subcluster 2 |
| PTGER3 | 5733 | PPI BAL Myeloid Subcluster 2 |
| RGS22 | 26166 | PPI BAL Myeloid Subcluster 2 |
| SAA1 | 6288 | PPI BAL Myeloid Subcluster 2 |
| SSTR2 | 6752 | PPI BAL Myeloid Subcluster 2 |
| SUCNR1 | 56670 | PPI BAL Myeloid Subcluster 2 |
| TAS2R13 | 50838 | PPI BAL Myeloid Subcluster 2 |
| ABI1 | 10006 | PPI BAL Myeloid Subcluster 7 |
| C1QA | 712 | PPI BAL Myeloid Subcluster 7 |
| C1QB | 713 | PPI BAL Myeloid Subcluster 7 |
| C1QC | 714 | PPI BAL Myeloid Subcluster 7 |
| C2 | 717 | PPI BAL Myeloid Subcluster 7 |
| CFH | 3075 | PPI BAL Myeloid Subcluster 7 |
| CRK | 1398 | PPI BAL Myeloid Subcluster 7 |
| DOCK1 | 1793 | PPI BAL Myeloid Subcluster 7 |
| FCGR2A | 2212 | PPI BAL Myeloid Subcluster 7 |
| FCGR3A | 2214 | PPI BAL Myeloid Subcluster 7 |
| GK | 2710 | PPI BAL Myeloid Subcluster 7 |
| IGSF6 | 10261 | PPI BAL Myeloid Subcluster 7 |
| KNDC1 | 85442 | PPI BAL Myeloid Subcluster 7 |
| LILRB4 | 11006 | PPI BAL Myeloid Subcluster 7 |
| MARCO | 8685 | PPI BAL Myeloid Subcluster 7 |
| MRC1 | 4360 | PPI BAL Myeloid Subcluster 7 |
| NCKAP1 | 10787 | PPI BAL Myeloid Subcluster 7 |
| PLA2G7 | 7941 | PPI BAL Myeloid Subcluster 7 |
| ANXA2 | 302 | PPI BAL Myeloid Subcluster 3 |
| APOE | 348 | PPI BAL Myeloid Subcluster 3 |
| C6 | 729 | PPI BAL Myeloid Subcluster 3 |
| C8B | 732 | PPI BAL Myeloid Subcluster 3 |
| CD44 | 960 | PPI BAL Myeloid Subcluster 3 |
| CP | 1356 | PPI BAL Myeloid Subcluster 3 |
| CREG1 | 8804 | PPI BAL Myeloid Subcluster 3 |
| CTSC | 1075 | PPI BAL Myeloid Subcluster 3 |
| DCP1B | 196513 | PPI BAL Myeloid Subcluster 3 |
| FABP5 | 2171 | PPI BAL Myeloid Subcluster 3 |
| FCGR1A | 2209 | PPI BAL Myeloid Subcluster 3 |
| FCGR1B | 2210 | PPI BAL Myeloid Subcluster 3 |
| FGFR2 | 2263 | PPI BAL Myeloid Subcluster 3 |
| FN1 | 2335 | PPI BAL Myeloid Subcluster 3 |
| FRK | 2444 | PPI BAL Myeloid Subcluster 3 |
| FTL | 2512 | PPI BAL Myeloid Subcluster 3 |
| FUCA1 | 2517 | PPI BAL Myeloid Subcluster 3 |
| GBP1 | 2633 | PPI BAL Myeloid Subcluster 3 |
| GBP6 | 163351 | PPI BAL Myeloid Subcluster 3 |
| GM2A | 2760 | PPI BAL Myeloid Subcluster 3 |
| GOLM1 | 51280 | PPI BAL Myeloid Subcluster 3 |
| HLA-DPA1 | 3113 | PPI BAL Myeloid Subcluster 3 |
| HLA-DQA1 | 3117 | PPI BAL Myeloid Subcluster 3 |
| HLA-DQA2 | 3118 | PPI BAL Myeloid Subcluster 3 |
| HLA-DQB2 | 3120 | PPI BAL Myeloid Subcluster 3 |
| IFI6 | 2537 | PPI BAL Myeloid Subcluster 3 |
| IFIH1 | 64135 | PPI BAL Myeloid Subcluster 3 |
| IFIT3 | 3437 | PPI BAL Myeloid Subcluster 3 |
| IFIT5 | 24138 | PPI BAL Myeloid Subcluster 3 |
| IFITM3 | 10410 | PPI BAL Myeloid Subcluster 3 |
| IGFBP3 | 3486 | PPI BAL Myeloid Subcluster 3 |
| IGFBP5 | 3488 | PPI BAL Myeloid Subcluster 3 |
| IL17RD | 54756 | PPI BAL Myeloid Subcluster 3 |
| IRF2 | 3660 | PPI BAL Myeloid Subcluster 3 |
| IRF6 | 3664 | PPI BAL Myeloid Subcluster 3 |
| ISG15 | 9636 | PPI BAL Myeloid Subcluster 3 |
| LAMB1 | 3912 | PPI BAL Myeloid Subcluster 3 |
| LGMN | 5641 | PPI BAL Myeloid Subcluster 3 |
| MGAT4A | 11320 | PPI BAL Myeloid Subcluster 3 |
| MID1 | 4281 | PPI BAL Myeloid Subcluster 3 |
| MMP1 | 4312 | PPI BAL Myeloid Subcluster 3 |
| MMP10 | 4319 | PPI BAL Myeloid Subcluster 3 |
| MMP7 | 4316 | PPI BAL Myeloid Subcluster 3 |
| MSLN | 10232 | PPI BAL Myeloid Subcluster 3 |
| MT2A | 4502 | PPI BAL Myeloid Subcluster 3 |
| NKAP | 79576 | PPI BAL Myeloid Subcluster 3 |
| OAS1 | 4938 | PPI BAL Myeloid Subcluster 3 |
| OAS2 | 4939 | PPI BAL Myeloid Subcluster 3 |
| OASL | 8638 | PPI BAL Myeloid Subcluster 3 |
| OSBPL1A | 114876 | PPI BAL Myeloid Subcluster 3 |
| PDCD1LG2 | 80380 | PPI BAL Myeloid Subcluster 3 |
| PRSS23 | 11098 | PPI BAL Myeloid Subcluster 3 |
| PTRH2 | 51651 | PPI BAL Myeloid Subcluster 3 |
| PYCARD | 29108 | PPI BAL Myeloid Subcluster 3 |
| S100A2 | 6273 | PPI BAL Myeloid Subcluster 3 |
| S100A7 | 6278 | PPI BAL Myeloid Subcluster 3 |
| SDC2 | 6383 | PPI BAL Myeloid Subcluster 3 |
| SDCBP | 6386 | PPI BAL Myeloid Subcluster 3 |
| SERPINB3 | 6317 | PPI BAL Myeloid Subcluster 3 |
| SERPINB4 | 6318 | PPI BAL Myeloid Subcluster 3 |
| SP110 | 3431 | PPI BAL Myeloid Subcluster 3 |
| SPARCL1 | 8404 | PPI BAL Myeloid Subcluster 3 |
| SPP1 | 6696 | PPI BAL Myeloid Subcluster 3 |
| TNC | 3371 | PPI BAL Myeloid Subcluster 3 |
| TRIM14 | 9830 | PPI BAL Myeloid Subcluster 3 |
| TRIM2 | 23321 | PPI BAL Myeloid Subcluster 3 |
| ACACB | 32 | PPI BAL Myeloid Subcluster 5 |
| ACADM | 34 | PPI BAL Myeloid Subcluster 5 |
| APOC1 | 341 | PPI BAL Myeloid Subcluster 5 |
| BDP1 | 55814 | PPI BAL Myeloid Subcluster 5 |
| BNIP3 | 664 | PPI BAL Myeloid Subcluster 5 |
| BRD8 | 10902 | PPI BAL Myeloid Subcluster 5 |
| CCL3 | 6348 | PPI BAL Myeloid Subcluster 5 |
| CEBPB | 1051 | PPI BAL Myeloid Subcluster 5 |
| CEP350 | 9857 | PPI BAL Myeloid Subcluster 5 |
| CNOT1 | 23019 | PPI BAL Myeloid Subcluster 5 |
| DEPTOR | 64798 | PPI BAL Myeloid Subcluster 5 |
| DHX32 | 55760 | PPI BAL Myeloid Subcluster 5 |
| EIF4EBP1 | 1978 | PPI BAL Myeloid Subcluster 5 |
| FABP4 | 2167 | PPI BAL Myeloid Subcluster 5 |
| FABP6 | 2172 | PPI BAL Myeloid Subcluster 5 |
| FAM120B | 84498 | PPI BAL Myeloid Subcluster 5 |
| FNIP1 | 96459 | PPI BAL Myeloid Subcluster 5 |
| FNIP2 | 57600 | PPI BAL Myeloid Subcluster 5 |
| GPN3 | 51184 | PPI BAL Myeloid Subcluster 5 |
| GRHL1 | 29841 | PPI BAL Myeloid Subcluster 5 |
| GTF3A | 2971 | PPI BAL Myeloid Subcluster 5 |
| GTF3C6 | 112495 | PPI BAL Myeloid Subcluster 5 |
| HMGCS2 | 3158 | PPI BAL Myeloid Subcluster 5 |
| ITPA | 3704 | PPI BAL Myeloid Subcluster 5 |
| KLF5 | 688 | PPI BAL Myeloid Subcluster 5 |
| LAMTOR5 | 10542 | PPI BAL Myeloid Subcluster 5 |
| LEP | 3952 | PPI BAL Myeloid Subcluster 5 |
| LPL | 4023 | PPI BAL Myeloid Subcluster 5 |
| MCTS1 | 28985 | PPI BAL Myeloid Subcluster 5 |
| MED11 | 400569 | PPI BAL Myeloid Subcluster 5 |
| MED19 | 219541 | PPI BAL Myeloid Subcluster 5 |
| MED21 | 9412 | PPI BAL Myeloid Subcluster 5 |
| MED7 | 9443 | PPI BAL Myeloid Subcluster 5 |
| MED8 | 112950 | PPI BAL Myeloid Subcluster 5 |
| NCOA2 | 10499 | PPI BAL Myeloid Subcluster 5 |
| NFIA | 4774 | PPI BAL Myeloid Subcluster 5 |
| NFIB | 4781 | PPI BAL Myeloid Subcluster 5 |
| NFIC | 4782 | PPI BAL Myeloid Subcluster 5 |
| NR2F2 | 7026 | PPI BAL Myeloid Subcluster 5 |
| PDE9A | 5152 | PPI BAL Myeloid Subcluster 5 |
| POLR3B | 55703 | PPI BAL Myeloid Subcluster 5 |
| POLR3C | 10623 | PPI BAL Myeloid Subcluster 5 |
| POLR3K | 51728 | PPI BAL Myeloid Subcluster 5 |
| PPARG | 5468 | PPI BAL Myeloid Subcluster 5 |
| PPARGC1A | 10891 | PPI BAL Myeloid Subcluster 5 |
| PRKAA1 | 5562 | PPI BAL Myeloid Subcluster 5 |
| PRKAA2 | 5563 | PPI BAL Myeloid Subcluster 5 |
| PRKAG1 | 5571 | PPI BAL Myeloid Subcluster 5 |
| PRKAG3 | 53632 | PPI BAL Myeloid Subcluster 5 |
| RGL1 | 23179 | PPI BAL Myeloid Subcluster 5 |
| RHEB | 6009 | PPI BAL Myeloid Subcluster 5 |
| RRAGA | 10670 | PPI BAL Myeloid Subcluster 5 |
| RRAGB | 10325 | PPI BAL Myeloid Subcluster 5 |
| RRAGC | 64121 | PPI BAL Myeloid Subcluster 5 |
| AIM2 | 9447 | PPI BAL Myeloid Subcluster 9 |
| CASP4 | 837 | PPI BAL Myeloid Subcluster 9 |
| CASP5 | 838 | PPI BAL Myeloid Subcluster 9 |
| IFI16 | 3428 | PPI BAL Myeloid Subcluster 9 |
| IL18 | 3606 | PPI BAL Myeloid Subcluster 9 |
| NLRC4 | 58484 | PPI BAL Myeloid Subcluster 9 |

Co-expressed Myeloid Subpopulations

| | |
|---|---|
| GeneSymbol | EntrezID |

CoV-2 PBMC Myeloid A1

| | |
|---|---|
| ADAMDEC1 | 27299 |
| ADGRE2 | 30817 |
| AIF1 | 199 |
| AOAH | 313 |
| APOBEC3B | 9582 |
| APOBR | 55911 |
| APOC1 | 341 |
| BST1 | 683 |
| C1QA | 712 |
| C1QB | 713 |
| C1QC | 714 |
| C1RL | 51279 |
| C2 | 717 |
| C4A | 720 |
| C4B | 721 |
| C4BPA | 722 |
| C5 | 727 |
| C6 | 729 |
| CCL18 | 6362 |
| CCL2 | 6347 |
| CCL7 | 6354 |
| CCL8 | 6355 |
| CD101 | 9398 |
| CD14 | 929 |
| CD163 | 9332 |
| CD209 | 30835 |
| CD300C | 10871 |
| CD300E | 342510 |
| CD300LF | 146722 |
| CD33 | 945 |
| CD68 | 968 |
| CD80 | 941 |
| CFD | 1675 |
| CFP | 5199 |
| CLEC10A | 10462 |
| CLEC12A | 160364 |
| CLEC12B | 387837 |
| CLEC16A | 23274 |
| CLEC4A | 50856 |
| CLEC4D | 338339 |
| CLEC4E | 26253 |
| CLEC5A | 23601 |
| CLEC6A | 93978 |
| CLEC7A | 64581 |
| CSF1R | 1436 |
| CSF2RA | 1438 |
| CSF2RB | 1439 |
| CSF3R | 1441 |
| CST3 | 1471 |
| CXCL10 | 3627 |
| CXCL11 | 6373 |
| CXCL2 | 2920 |
| CXCL9 | 4283 |
| CYBA | 1535 |
| CYBB | 1536 |
| FCER1G | 2207 |
| FCGR1A | 2209 |
| FCGR1B | 2210 |
| FCGR2A | 2212 |
| FCGR2B | 2213 |
| FCGR2C | 9103 |
| FGR | 2268 |
| FLT3 | 2322 |
| FOLR2 | 2350 |
| FUT4 | 2526 |
| GRN | 2896 |
| HVCN1 | 84329 |
| IFI30 | 10437 |
| IGSF6 | 10261 |
| IL10RA | 3587 |
| IL18 | 3606 |
| IL1A | 3552 |
| IL1RAP | 3556 |
| IL1RN | 3557 |
| ITGAX | 3687 |
| LGALS12 | 85329 |
| LGALS9 | 3965 |
| LILRA1 | 11024 |
| LILRA2 | 11027 |
| LILRA5 | 353514 |
| LILRA6 | 79168 |
| LILRB1 | 10859 |
| LILRB2 | 10288 |
| LILRB3 | 11025 |
| LILRB4 | 11006 |
| LILRB5 | 10990 |
| LMNB1 | 4001 |
| LY86 | 9450 |
| LYVE1 | 10894 |
| LYZ | 4069 |
| MARCO | 8685 |
| MEFV | 4210 |
| MERTK | 10461 |
| MFGE8 | 4240 |
| MNDA | 4332 |
| MPEG1 | 219972 |
| MRC1 | 4360 |
| MS4A4A | 51338 |
| MS4A6A | 64231 |
| MSR1 | 4481 |
| NLRP12 | 91662 |
| NLRP3 | 114548 |
| NOD2 | 64127 |
| NTSR1 | 4923 |
| OSCAR | 126014 |
| OTOF | 9381 |
| PECAM1 | 5175 |
| PILRA | 29992 |
| PLEK | 5341 |
| PRAM1 | 84106 |
| RNASE3 | 6037 |
| RNASE6 | 6039 |
| RNASE7 | 84659 |
| S100A8 | 6279 |
| S100A9 | 6280 |
| SCARB1 | 949 |
| SECTM1 | 6398 |
| SEMA4A | 64218 |
| SERPINB9 | 5272 |
| SERPING1 | 710 |
| SIGLEC1 | 6614 |
| SIGLEC10 | 89790 |
| SIGLEC5 | 8778 |
| SIGLEC7 | 27036 |
| SKAP2 | 8935 |
| SLC11A1 | 6556 |
| SLITRK4 | 139065 |
| SPI1 | 6688 |
| TGM2 | 7052 |
| THBD | 7056 |
| TLR2 | 7097 |
| TLR8 | 51311 |
| TNF | 7124 |
| TNFAIP8L2 | 79626 |
| TNFRSF1B | 7133 |
| TNFSF13B | 10673 |
| TREM1 | 54210 |
| TREML4 | 285852 |
| TYROBP | 7305 |
| UNC93B1 | 81622 |
| VENTX | 27287 |
| VSIG4 | 11326 |
| VSTM1 | 284415 |

CoV-2 Lung Myeloid A2

| | |
|---|---|
| ADAMDEC1 | 27299 |
| ADGRE1 | 2015 |
| ADGRE2 | 30817 |
| AIF1 | 199 |
| APOBEC3B | 9582 |
| APOBEC3G | 60489 |
| APOBR | 55911 |
| APOC1 | 341 |
| BST1 | 683 |
| C1QA | 712 |
| C1QB | 713 |
| C1QC | 714 |
| C2 | 717 |
| C4BPA | 722 |
| C4BPB | 725 |
| C6 | 729 |
| C8A | 731 |
| CCL18 | 6362 |
| CCL2 | 6347 |
| CCL22 | 6367 |
| CCL7 | 6354 |
| CCL8 | 6355 |
| CD14 | 929 |
| CD300E | 342510 |
| CD33 | 945 |
| CD80 | 941 |
| CFD | 1675 |
| CFP | 5199 |
| CHI3L1 | 1116 |
| CHIT1 | 1118 |
| CLEC12A | 160364 |
| CLEC12B | 387837 |
| CLEC1A | 51267 |
| CLEC2B | 9976 |
| CLEC4A | 50856 |
| CLEC4D | 338339 |
| CLEC4E | 26253 |
| CLEC7A | 64581 |
| CSF2 | 1437 |
| CSF2RB | 1439 |
| CSF3R | 1441 |
| CST3 | 1471 |
| CXCL1 | 2919 |
| CXCL10 | 3627 |
| CXCL11 | 6373 |
| CXCL13 | 10563 |
| CXCL8 | 3576 |
| CXCR2 | 3579 |
| CYBA | 1535 |
| CYBB | 1536 |
| FCER1G | 2207 |
| FCGR1A | 2209 |
| FCGR2A | 2212 |
| FCGR2B | 2213 |
| FCGR2C | 9103 |
| FCGR3A | 2214 |
| FCGR3B | 2215 |
| FFAR2 | 2867 |
| FGR | 2268 |
| FOLR2 | 2350 |
| GRN | 2896 |
| IFNL1 | 282618 |
| IGSF6 | 10261 |
| IL12B | 3593 |
| IL18 | 3606 |
| IL18RAP | 8807 |
| IL1A | 3552 |
| IL1B | 3553 |
| IL1RAP | 3556 |
| IL1RN | 3557 |
| IL20 | 50604 |
| IL23A | 51561 |
| IL27 | 246778 |
| ITGAX | 3687 |
| LAMP3 | 27074 |
| LGALS9 | 3965 |
| LGALS9C | 654346 |
| LILRA5 | 353514 |
| LILRA6 | 79168 |
| LILRB1 | 10859 |
| LILRB2 | 10288 |
| LILRB3 | 11025 |
| LILRB4 | 11006 |
| LILRB5 | 10990 |
| LMNB1 | 4001 |
| LY86 | 9450 |
| LYZ | 4069 |
| MNDA | 4332 |
| MPEG1 | 219972 |
| MS4A4A | 51338 |
| MS4A6A | 64231 |
| MSR1 | 4481 |
| NLRP3 | 114548 |
| OLR1 | 4973 |
| OTOF | 9381 |
| PDCD1LG2 | 80380 |
| PILRA | 29992 |
| PLEK | 5341 |
| S100A8 | 6279 |
| S100A9 | 6280 |
| S1PR5 | 53637 |
| SECTM1 | 6398 |
| SEMA4A | 64218 |
| SERPINB9 | 5272 |
| SERPING1 | 710 |
| SIGLEC14 | 1E+08 |
| SIGLEC5 | 8778 |
| SLPI | 6590 |
| SPI1 | 6688 |
| TLR2 | 7097 |
| TNF | 7124 |
| TNFSF13B | 10673 |
| TNFSF14 | 8740 |
| TNIP3 | 79931 |
| TREM1 | 54210 |
| TREML4 | 285852 |
| TYROBP | 7305 |
| UNC93B1 | 81622 |
| VSIG4 | 11326 |

CoV-2 BAL Lung Myeloid A3

| GeneSymbol | EntrezID |
|---|---|
| ACE | 1636 |
| ADAMDEC1 | 27299 |
| ADGRE3 | 84658 |
| AIF1 | 199 |
| APOBEC3G | 60489 |
| APOC1 | 341 |
| C1QA | 712 |
| C1QB | 713 |
| C1QC | 714 |
| C1R | 715 |
| C2 | 717 |
| C4BPA | 722 |
| C5 | 727 |
| C6 | 729 |
| CCL18 | 6362 |
| CCL2 | 6347 |
| CCL28 | 56477 |
| CCL7 | 6354 |
| CCL8 | 6355 |
| CD163 | 9332 |
| CD300LF | 146722 |
| CD5L | 922 |
| CD68 | 968 |
| CD80 | 941 |
| CHI3L1 | 1116 |
| CLEC2B | 9976 |
| CLEC4E | 26253 |
| CLEC5A | 23601 |
| CLEC6A | 93978 |
| CXCL1 | 2919 |
| CXCL10 | 3627 |
| CXCL11 | 6373 |
| CXCL2 | 2920 |
| CXCL8 | 3576 |
| CXCL9 | 4283 |
| CXCR2 | 3579 |
| FCER1G | 2207 |
| FCGR1A | 2209 |
| FCGR1B | 2210 |
| FCGR2A | 2212 |
| FCGR2C | 9103 |
| FCGR3A | 2214 |
| FCGR3B | 2215 |
| FOLR2 | 2350 |
| IGSF6 | 10261 |
| IK | 3550 |
| IL18 | 3606 |
| IL1A | 3552 |
| IL1B | 3553 |
| IL1RN | 3557 |
| IL23A | 51561 |
| LAMP3 | 27074 |
| LGALS9B | 284194 |
| LGALS9C | 654346 |
| LILRB4 | 11006 |
| LILRB5 | 10990 |
| LY86 | 9450 |
| LYVE1 | 10894 |
| MARCO | 8685 |
| MERTK | 10461 |
| MNDA | 4332 |
| MRC1 | 4360 |
| MS4A4A | 51338 |
| MSR1 | 4481 |
| OLR1 | 4973 |
| OTOF | 9381 |
| PDCD1LG2 | 80380 |
| PILRA | 29992 |
| RNASE7 | 84659 |
| SCARB1 | 949 |
| SERPING1 | 710 |
| SIGLEC1 | 6614 |
| SIGLEC14 | 100049587 |
| SIGLEC5 | 8778 |
| SIGLEC7 | 27036 |
| SKAP2 | 8935 |
| SLAMF8 | 56833 |
| SLPI | 6590 |
| STAP2 | 55620 |
| TGM2 | 7052 |
| TLR8 | 51311 |
| TNF | 7124 |
| TNFAIP8L2 | 79626 |
| TNFSF13B | 10673 |
| TNIP3 | 79931 |
| TYROBP | 7305 |
| VSIG4 | 11326 |

CoV-2 PBMC Myeloid B1

| GeneSymbol | EntrezID |
|---|---|
| ACE | 1636 |
| ADAM8 | 101 |
| ADGRE1 | 2015 |
| ADGRE3 | 84658 |
| APOBEC3G | 60489 |
| C1R | 715 |
| CCL28 | 56477 |
| CD5L | 922 |
| CHI3L1 | 1116 |
| CHIT1 | 1118 |
| CLEC2B | 9976 |
| CXCL1 | 2919 |
| CXCL8 | 3576 |
| CXCR2 | 3579 |
| FCER1A | 2205 |
| FCGR3A | 2214 |
| FCGR3B | 2215 |
| FFAR2 | 2867 |
| IK | 3550 |
| IL18RAP | 8807 |
| IL1B | 3553 |
| IL23A | 51561 |
| LAMP3 | 27074 |
| LGALS9B | 284194 |
| LGALS9C | 654346 |
| OLR1 | 4973 |
| PDCD1LG2 | 80380 |
| PYHIN1 | 149628 |
| S1PR5 | 53637 |
| SIGLEC14 | 1E+08 |
| SLAMF8 | 56833 |
| SLPI | 6590 |
| STAP2 | 55620 |
| TNFSF14 | 8740 |
| TNIP3 | 79931 |
| TREML2 | 79865 |

CoV-2 PBMC Lung B2

| GeneSymbol | EntrezID |
|---|---|
| ACE | 1636 |
| ADAM8 | 101 |
| AOAH | 313 |
| ART4 | 420 |
| BMX | 660 |
| C1R | 715 |
| C1RL | 51279 |
| C4A | 720 |
| C4B | 721 |
| CD163 | 9332 |
| CD209 | 30835 |
| CD300C | 10871 |
| CD300LF | 146722 |
| CD5L | 922 |
| CFB | 629 |
| CLEC10A | 10462 |
| CLEC16A | 23274 |
| CLEC5A | 23601 |
| CSF1R | 1436 |
| CSF2RA | 1438 |
| CXCL2 | 2920 |
| CXCL9 | 4283 |
| FCER1A | 2205 |
| FCGR1B | 2210 |
| FUT4 | 2526 |
| HVCN1 | 84329 |
| IK | 3550 |
| IL10RA | 3587 |
| IL12A | 3592 |
| IL31RA | 133396 |
| LGALS9B | 284194 |
| LILRA1 | 11024 |
| LILRA2 | 11027 |
| LYVE1 | 10894 |
| MARCO | 8685 |
| MEFV | 4210 |
| MERTK | 10461 |
| MFGE8 | 4240 |
| MRC1 | 4360 |
| NLRP12 | 91662 |
| NOD2 | 64127 |
| OSCAR | 126014 |
| PECAM1 | 5175 |
| PLA2G5 | 5322 |
| PRAM1 | 84106 |
| PYHIN1 | 149628 |
| RNASE6 | 6039 |
| SCARB1 | 949 |
| SIGLEC1 | 6614 |
| SKAP2 | 8935 |
| SLC11A1 | 6556 |
| STAP2 | 55620 |
| TEK | 7010 |
| TGM2 | 7052 |
| THBD | 7056 |
| TLR8 | 51311 |
| TNFAIP8L2 | 79626 |
| TNFRSF1B | 7133 |

CoV-2 BAL Myeloid B3

| GeneSymbol | EntrezID |
|---|---|
| ADAM8 | 101 |
| ADGRE1 | 2015 |
| ADGRE2 | 30817 |
| AOAH | 313 |
| APOBEC3B | 9582 |
| APOBR | 55911 |
| BST1 | 683 |
| C1RL | 51279 |
| C4A | 720 |
| C4B | 721 |
| CD101 | 9398 |
| CD14 | 929 |
| CD209 | 30835 |
| CD300C | 10871 |
| CD300E | 342510 |
| CD33 | 945 |
| CFD | 1675 |
| CFP | 5199 |
| CHIT1 | 1118 |
| CLEC10A | 10462 |
| CLEC12A | 160364 |
| CLEC12B | 387837 |
| CLEC16A | 23274 |
| CLEC4A | 50856 |
| CLEC4D | 338339 |
| CLEC7A | 64581 |
| CSF1R | 1436 |
| CSF2RA | 1438 |
| CSF2RB | 1439 |
| CSF3R | 1441 |
| CST3 | 1471 |
| CYBA | 1535 |
| CYBB | 1536 |
| FCER1A | 2205 |
| FCGR2B | 2213 |
| FFAR2 | 2867 |
| FGR | 2268 |
| FLT3 | 2322 |
| FUT4 | 2526 |
| GRN | 2896 |
| HVCN1 | 84329 |
| IFI30 | 10437 |
| IL10RA | 3587 |
| IL18RAP | 8807 |
| IL1RAP | 3556 |
| ITGAX | 3687 |
| LGALS12 | 85329 |
| LGALS9 | 3965 |
| LILRA1 | 11024 |
| LILRA2 | 11027 |
| LILRA5 | 353514 |
| LILRA6 | 79168 |
| LILRB1 | 10859 |
| LILRB2 | 10288 |
| LILRB3 | 11025 |
| LMNB1 | 4001 |
| LYZ | 4069 |
| MEFV | 4210 |
| MFGE8 | 4240 |
| MPEG1 | 219972 |
| MS4A6A | 64231 |
| NLRP12 | 91662 |
| NLRP3 | 114548 |
| NOD2 | 64127 |
| NTSR1 | 4923 |
| OSCAR | 126014 |
| PECAM1 | 5175 |
| PLEK | 5341 |
| PRAM1 | 84106 |
| PYHIN1 | 149628 |
| RNASE3 | 6037 |
| RNASE6 | 6039 |
| S100A8 | 6279 |
| S100A9 | 6280 |
| S1PR5 | 53637 |
| SECTM1 | 6398 |
| SEMA4A | 64218 |
| SERPINB9 | 5272 |
| SIGLEC10 | 89790 |
| SLC11A1 | 6556 |
| SLITRK4 | 139065 |
| SPI1 | 6688 |
| THBD | 7056 |
| TLR2 | 7097 |
| TNFRSF1B | 7133 |
| TNFSF14 | 8740 |
| TREM1 | 54210 |
| TREML2 | 79865 |
| TREML4 | 285852 |
| UNC93B1 | 81622 |
| VENTX | 27287 |
| VSTM1 | 284415 |

Co-Expressed Myeloid Subpopulations Venn Diagrams

PBMC/Lung/BAL

| GeneSymbol | EntrezID |
|---|---|
| ADAMDEC1 | 27299 |
| AIF1 | 199 |
| APOC1 | 341 |
| C1QA | 712 |
| C1QB | 713 |
| C1QC | 714 |
| C2 | 717 |
| C4BPA | 722 |
| C6 | 729 |
| CCL18 | 6362 |
| CCL2 | 6347 |
| CCL7 | 6354 |
| CCL8 | 6355 |
| CD80 | 941 |
| CLEC4E | 26253 |
| CXCL10 | 3627 |
| CXCL11 | 6373 |
| FCER1G | 2207 |
| FCGR1A | 2209 |
| FCGR2A | 2212 |
| FCGR2C | 9103 |
| FOLR2 | 2350 |
| IGSF6 | 10261 |
| IL18 | 3606 |
| IL1A | 3552 |
| IL1RN | 3557 |
| LILRB4 | 11006 |
| LILRB5 | 10990 |
| LY86 | 9450 |
| MNDA | 4332 |
| MS4A4A | 51338 |
| MSR1 | 4481 |
| OTOF | 9381 |
| PILRA | 29992 |
| SERPING1 | 710 |
| SIGLEC5 | 8778 |
| TNF | 7124 |
| TNFSF13B | 10673 |
| TYROBP | 7305 |
| VSIG4 | 11326 |

PBMC/Lung

| GeneSymbol | EntrezID |
|---|---|
| ADGRE2 | 30817 |
| APOBEC3B | 9582 |
| APOBR | 55911 |
| BST1 | 683 |
| CD14 | 929 |
| CD300E | 342510 |
| CD33 | 945 |
| CFD | 1675 |
| CFP | 5199 |
| CLEC12A | 160364 |
| CLEC12B | 387837 |
| CLEC4A | 50856 |
| CLEC4D | 338339 |
| CLEC7A | 64581 |
| CSF2RB | 1439 |
| CSF3R | 1441 |
| CST3 | 1471 |
| CYBA | 1535 |
| CYBB | 1536 |
| FCGR2B | 2213 |
| FGR | 2268 |
| GRN | 2896 |
| IL1RAP | 3556 |
| ITGAX | 3687 |
| LGALS9 | 3965 |
| LILRA5 | 353514 |
| LILRA6 | 79168 |
| LILRB1 | 10859 |
| LILRB2 | 10288 |
| LILRB3 | 11025 |
| LMNB1 | 4001 |
| LYZ | 4069 |
| MPEG1 | 219972 |
| MS4A6A | 64231 |
| NLRP3 | 114548 |
| PLEK | 5341 |
| S100A8 | 6279 |
| S100A9 | 6280 |
| SECTM1 | 6398 |
| SEMA4A | 64218 |
| SERPINB9 | 5272 |
| SPI1 | 6688 |
| TLR2 | 7097 |
| TREM1 | 54210 |
| TREML4 | 285852 |
| UNC93B1 | 81622 |

PBMC/BAL

| GeneSymbol | EntrezID |
|---|---|
| C5 | 727 |
| CD163 | 9332 |
| CD300LF | 146722 |
| CD68 | 968 |
| CLEC5A | 23601 |
| CLEC6A | 93978 |
| CXCL2 | 2920 |
| CXCL9 | 4283 |
| FCGR1B | 2210 |
| LYVE1 | 10894 |
| MARCO | 8685 |
| MERTK | 10461 |
| MRC1 | 4360 |
| RNASE7 | 84659 |
| SCARB1 | 949 |
| SIGLEC1 | 6614 |
| SIGLEC7 | 27036 |
| SKAP2 | 8935 |
| TGM2 | 7052 |
| TLR8 | 51311 |
| TNFAIP8L2 | 79626 |

LUNG/BAL

| GeneSymbol | EntrezID |
|---|---|
| APOBEC3G | 60489 |
| CHI3L1 | 1116 |
| CLEC2B | 9976 |
| CXCL1 | 2919 |
| CXCL8 | 3576 |
| CXCR2 | 3579 |
| FCGR3A | 2214 |
| FCGR3B | 2215 |
| IL1B | 3553 |
| IL23A | 51561 |
| LAMP3 | 27074 |
| LGALS9C | 654346 |
| OLR1 | 4973 |
| PDCD1LG2 | 80380 |
| SIGLEC14 | 1E+08 |
| SLPI | 6590 |
| TNIP3 | 79931 |

PBMC

| GeneSymbol | EntrezID |
|---|---|
| AOAH | 313 |
| C1RL | 51279 |
| C4A | 720 |
| C4B | 721 |
| CD101 | 9398 |
| CD209 | 30835 |
| CD300C | 10871 |
| CLEC10A | 10462 |
| CLEC16A | 23274 |
| CSF1R | 1436 |
| CSF2RA | 1438 |
| FLT3 | 2322 |
| FUT4 | 2526 |
| HVCN1 | 84329 |
| IFI30 | 10437 |
| IL10RA | 3587 |
| LGALS12 | 85329 |
| LILRA1 | 11024 |
| LILRA2 | 11027 |
| MEFV | 4210 |
| MFGE8 | 4240 |
| NLRP12 | 91662 |
| NOD2 | 64127 |
| NTSR1 | 4923 |
| OSCAR | 126014 |
| PECAM1 | 5175 |
| PRAM1 | 84106 |
| RNASE3 | 6037 |
| RNASE6 | 6039 |
| SIGLEC10 | 89790 |
| SLC11A1 | 6556 |
| SLITRK4 | 139065 |
| THBD | 7056 |
| TNFRSF1B | 7133 |
| VENTX | 27287 |
| VSTM1 | 284415 |

LUNG

| GeneSymbol | EntrezID |
|---|---|
| ADGRE1 | 2015 |
| C4BPB | 725 |
| C8A | 731 |
| CCL22 | 6367 |
| CHIT1 | 1118 |
| CLEC1A | 51267 |
| CSF2 | 1437 |
| CXCL13 | 10563 |
| FFAR2 | 2867 |
| IFNL1 | 282618 |
| IL12B | 3593 |
| IL18RAP | 8807 |
| IL20 | 50604 |
| IL27 | 246778 |
| S1PR5 | 53637 |
| TNFSF14 | 8740 |

BAL

| GeneSymbol | EntrezID |
|---|---|
| ACE | 1636 |
| ADGRE3 | 84658 |
| C1R | 715 |
| CCL28 | 56477 |
| CD5L | 922 |
| IK | 3550 |
| LGALS9B | 284194 |
| SLAMF8 | 56833 |
| STAP2 | 55620 |

### Example 2: Comprehensive Transcriptomic Analysis of COVID-19 Blood, Lung, and Airway

SARS-CoV2 may refer to an uncharacterized coronavirus and causative agent of the COVID-19 pandemic. The host response to SARS-CoV2 has not yet been fully delineated, hampering a precise approach to therapy. To address this, a comprehensive analysis of gene expression data from the blood, lung, and airway of COVID-19 patients was performed. The results obtained indicate that COVID-19 pathogenesis is driven by populations of myeloid-lineage cells with highly inflammatory but distinct transcriptional signatures in each compartment. The relative absence of cytotoxic cells in the lung indicates a model in which delayed clearance of the virus may permit exaggerated myeloid cell activation that contributes to disease pathogenesis by the production of inflammatory mediators. The gene expression profiles may also identify potential therapeutic targets that may be modified with available drugs. The results and data indicate that transcriptomic profiling can provide an understanding of the pathogenesis of COVID-19 in individual patients.

Coronaviruses (CoV) generally refer to a group of enveloped, single, positive-stranded RNA viruses causing mild to severe respiratory illnesses in humans (Refs. 1-3). In the past two decades, three worldwide outbreaks have originated from CoVs capable of infecting the lower respiratory tract, resulting in heightened pathogenicity and high mortality rates. There is currently a global pandemic stemming from a third CoV strain, severe acute respiratory syndrome coronavirus 2 (SARS-CoV2), the causative agent of coronavirus disease 2019 (COVID-19). In the majority of cases, patients exhibit mild symptoms, whereas in more severe cases, patients may develop severe lung injury and death from respiratory failure (Refs. 4-5).

At this time, there is still incomplete information available regarding the host response to SARS-CoV2 infection and the perturbations resulting in a severe outcome. Despite this, clues can be derived from our knowledge of the immune response to infection by other respiratory viruses, including other CoVs. After infection, viruses are typically detected by pattern recognition receptors (PRRs) such as the inflammasome sensor NLRP3, which signal the release of interferons (IFNs) and inflammatory cytokines including the IL-1 family, IL-6, and TNF, that activate a local and systemic response to infection (Refs. 6-7). This involves the recruitment, activation, and differentiation of innate and adaptive immune cells, including neutrophils, inflammatory myeloid cells, CD8 T cells, and natural killer (NK) cells (Ref 8). Resolution of infection may be largely dependent on the cytotoxic activity of CD8 T cells and NK cells, which enable clearance of virus-infected cells (Ref 8). Failure to clear virus-infected cells may facilitate a hyper-inflammatory state termed Macrophage (M1) activation syndrome (MAS) or “cytokine storm”, and ultimately damage to the infected lung (Refs. 9-10).

The recent emergence of SARS-CoV2 and the relative lack of comprehensive knowledge regarding the progression of COVID-19 disease has constrained the ability to develop effective treatments for infected patients. One approach to obtain a more complete understanding of the host response to SARS-CoV2 is to examine gene expression in relevant tissues. A limited number of gene expression profiles may be available from patients with COVID-19 and have yielded some insights into the pathogenic processes triggered by infection with SARS-CoV2 (Refs. 11-13). However, because of the small number of samples and limited analysis, a comprehensive understanding of the biological state of SARS-CoV2-affected tissues may not be available. Recognizing this need, the present disclosure provides an analysis and assessment of available SARS-CoV2 gene expression datasets from blood, lung, and airway using a number of orthogonal bioinformatic tools to provide a more complete view of the nature of the COVID-19 inflammatory response and the potential points of therapeutic intervention.

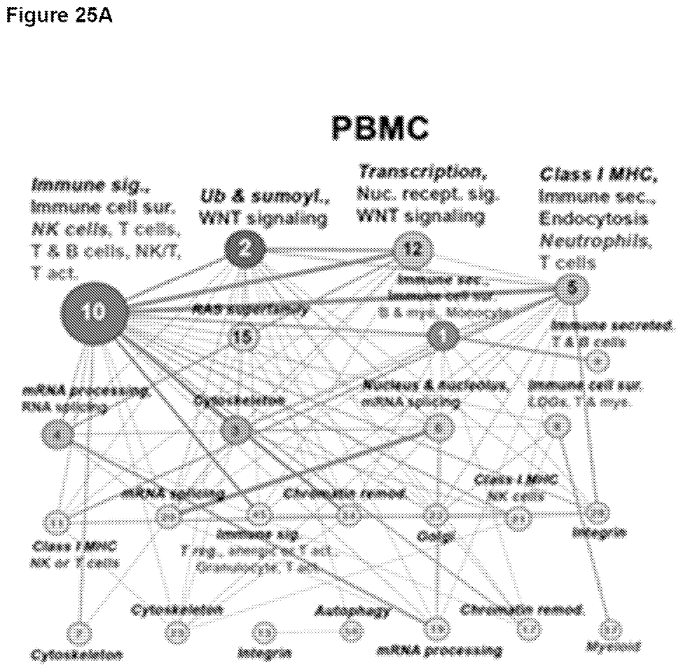

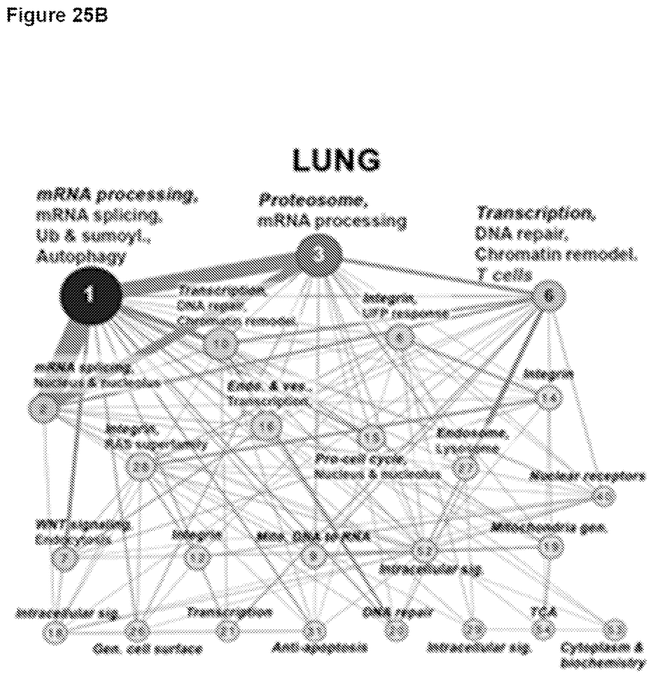

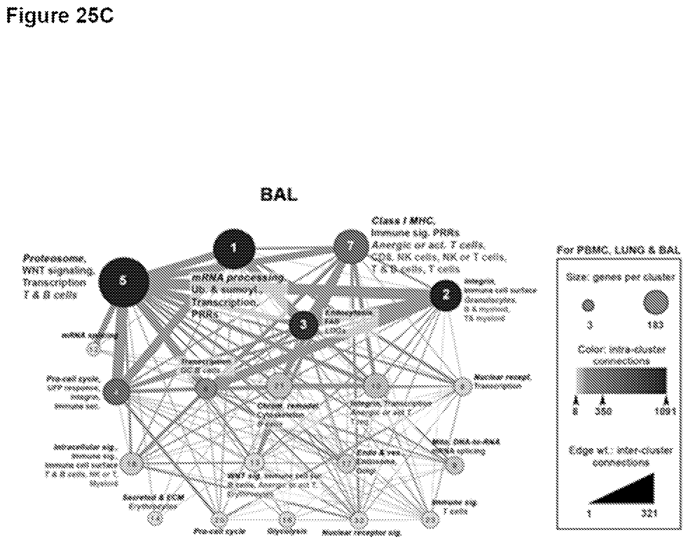

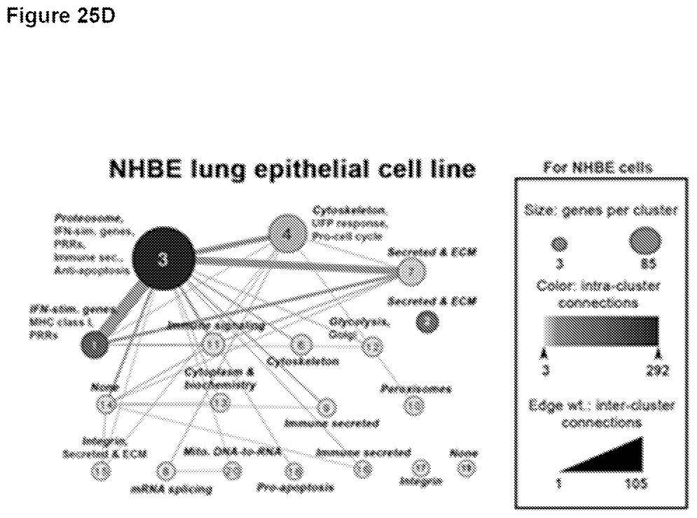

Gene expression analysis of blood, lung, and airway of COVID-19 patients was performed as follows. To characterize the pathologic response to SARS-CoV2 infection, transcriptomic data was analyzed from peripheral blood mononuclear cells (PBMCs) and postmortem lung tissue of COVID-19 patients and healthy controls as well as bronchoalveolar lavage (BAL) fluid of COVID-19 patients (CRA002390, GSE147507, FIGS. 25A-25D) (Refs. 11-12). First, changes in gene expression were determined in the blood (PBMC-CTL vs PBMC-CoV2) and lung (Lung-CTL vs Lung-CoV2). Because no control BAL fluids were associated with the BAL-CoV2 samples, BAL-CoV2 was compared to PBMC-CoV2 from the same dataset to avoid effects related to batch and methodology. Overall, a set of 4,245 differentially expressed genes (DEGs) in the blood (2,166 up and 2,079 down), 2,220 DEGs in the lung (684 up and 1,536 down), and 8,952 DEGs in the airway (BAL) (4,052 up and 4,900 down) were determined (Table 6).

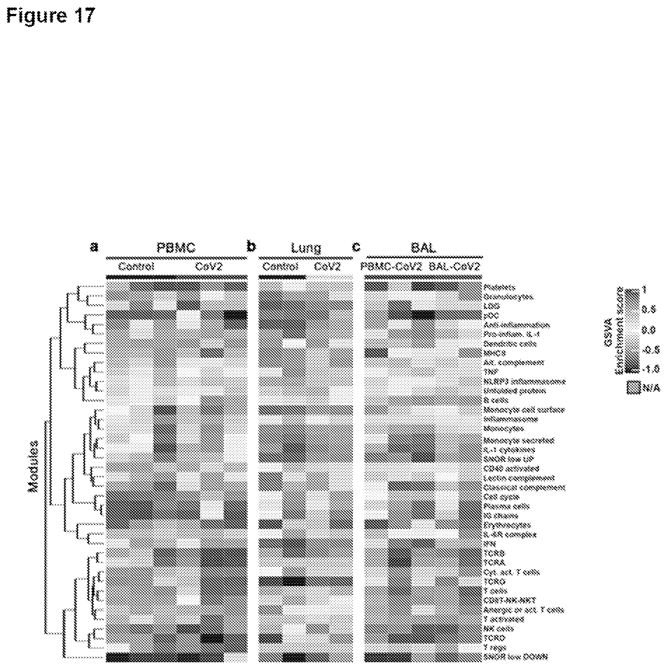

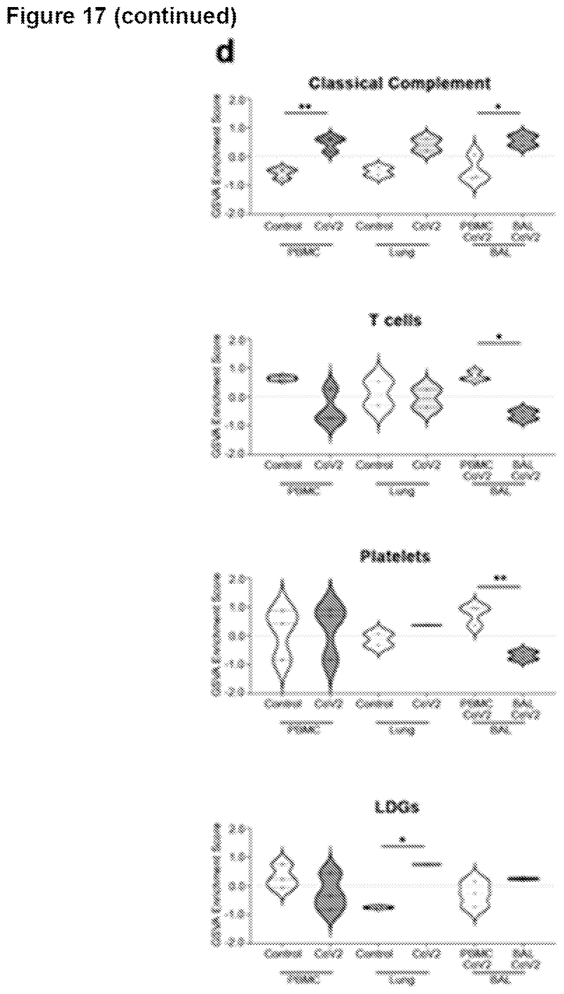

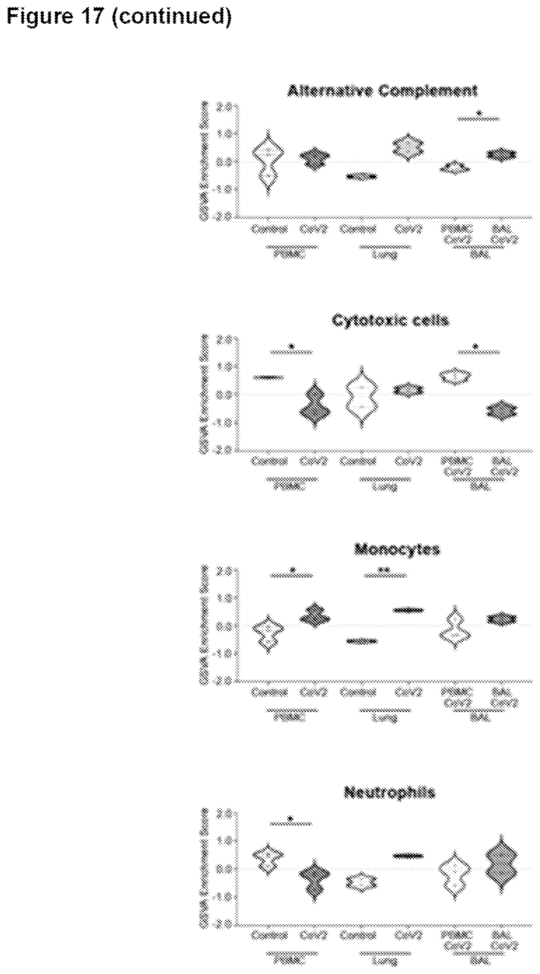

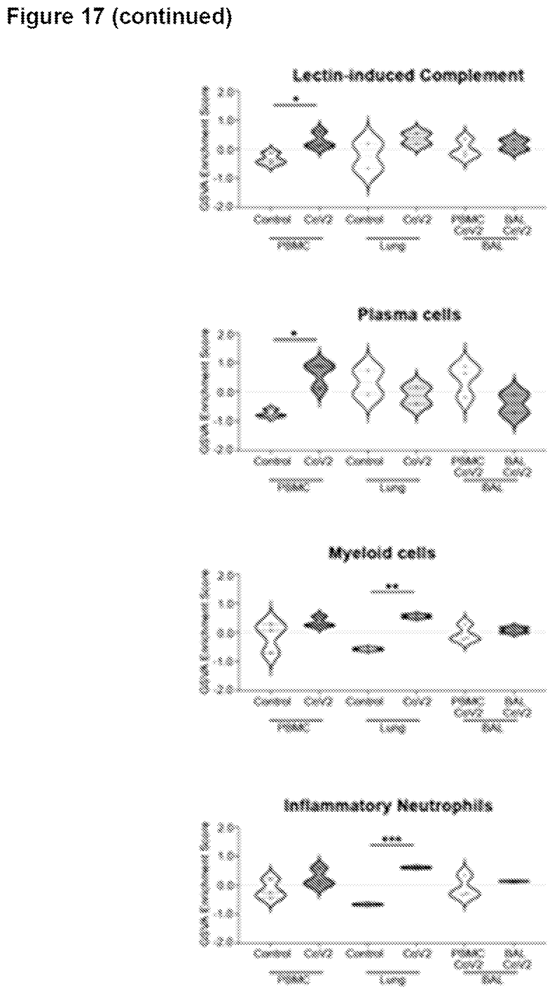

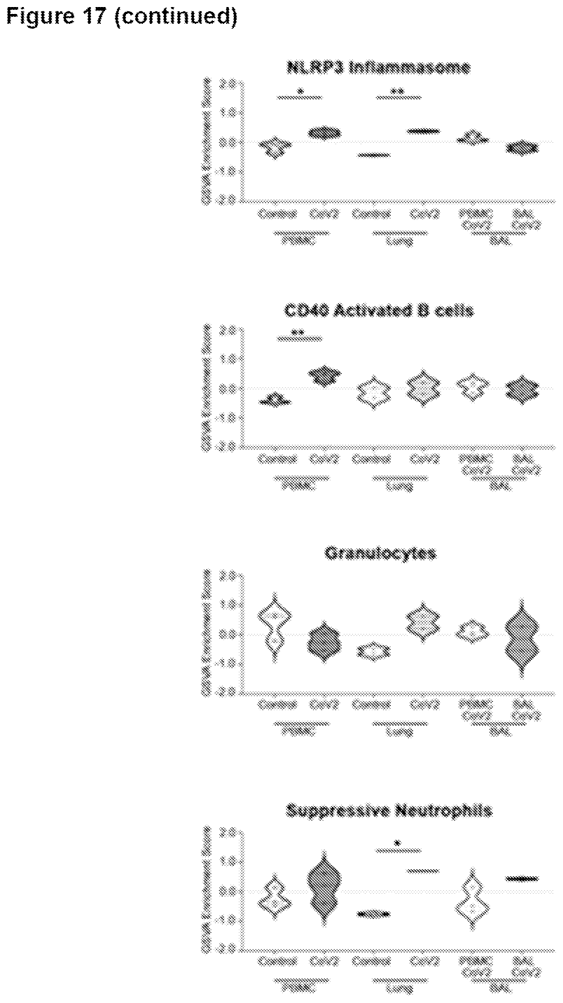

Conserved and differential enrichment of inflammatory cells and pathways in COVID-19 patients was analyzed as follows. To interrogate pathologic pathways in the 3 compartments, Gene Set Variation Analysis (GSVA) was performed utilizing a number of informative gene modules (Refs. 14-15) (FIG. 17). Numerous innate immune response pathways were increased in all 3 compartments, whereas adaptive immune signatures tended to be decreased in blood and airway, but not lung (Refs. 11-12 and 16). Although heterogeneity was observed in all compartments, closer examination revealed consistent perturbations. In the blood, the classical and lectin-induced complement pathways, as well as the NLRP3 inflammasome, plasma cells (PCs), and monocytes (Mo) were significantly enriched in COVID-19 patients, whereas cytotoxic cells and neutrophils were decreased. In the lung, the NLRP3 inflammasome, Mo and myeloid cells were enriched in COVID-19 patients. In addition, although the general granulocyte signature was not significantly increased, a specific low-density granulocyte (LDG) signature (Ref 17) and gene sets of inflammatory and suppressive neutrophils derived from COVID-19 blood were enriched in the lung (Refs. 18-19). In the airway, the classical and alternative complement pathways were enriched and T cells and cytotoxic cells were decreased.

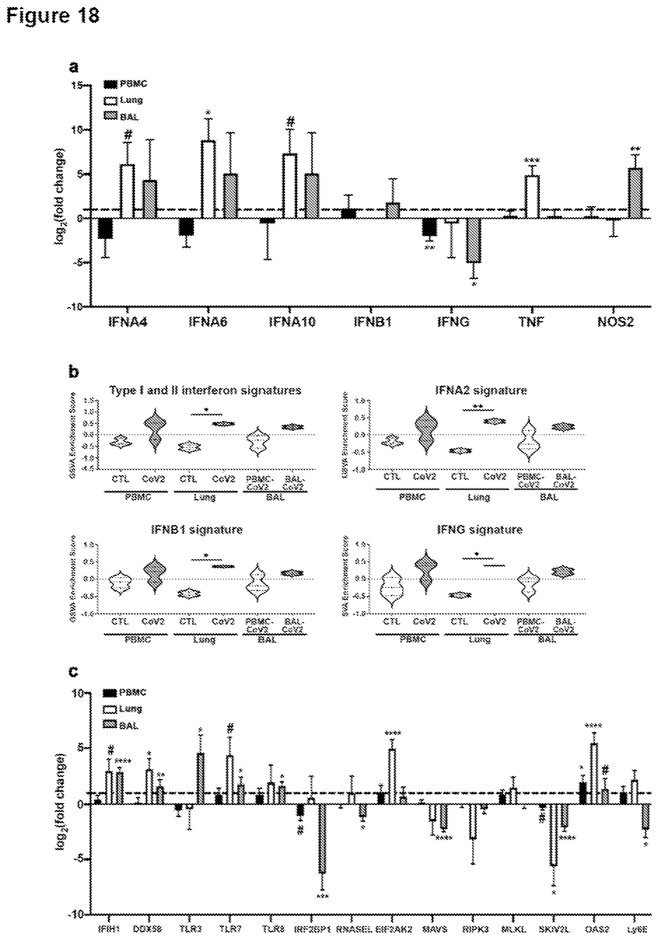

Although there is conflicting data on the presence of an IFN gene signature (IGS) and whether SARS-CoV2 infection induces a robust IFN response (Refs. 12-13 and 20), increased expression was observed of Type I IFN genes (IFNA4, IFNA6, IFNA10), and significant enrichment was observed of the common Type I and Type II IGS, including enrichment of IFNA2, IFNB1 and IFNG gene signatures specifically in the lung tissue (FIGS. 18A-18B). Furthermore, increased expression was detected of genes found to be important for the anti-viral innate immune response (IFIH1, DDX58, EIF2AK2, OAS2), and decreased expression was detected of negative regulators of this response (IRF2BP1, SKIV2L) in both the lung and airway compartments (FIG. 18C) (Ref 21). Interestingly, expression of MAVS, a signaling adaptor for RNA virus sensors, was observed to be decreased in the airway, which is consistent with the reported effect of SARS-CoV2 and may reflect a mechanism of viral immune evasion (Refs. 22-23).

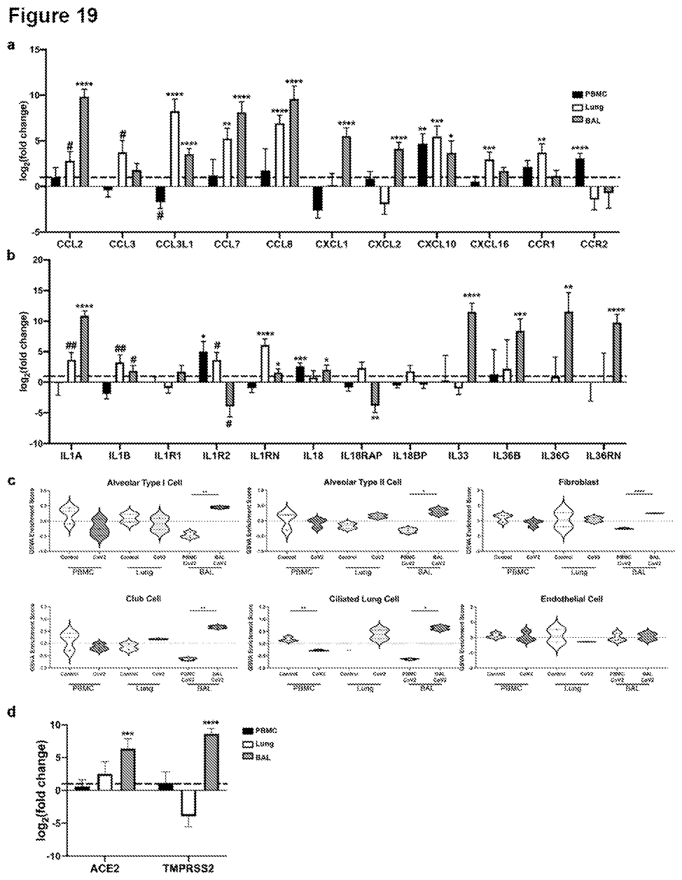

Increased expression of inflammatory mediators in the lungs of COVID-19 patients was analyzed as follows. To examine the nature of the inflammatory response in the tissue compartments in greater detail, specific DEGs of interest (FIG. 19, sections a and b) were examined. In the blood, increased expression was observed of the inflammatory chemokine CXCL10, which is an IFNG response gene and involved in the activation and chemotaxis of peripheral immune cells, (Ref 24), the chemokine receptor CCR2, which may be critical for immune cell recruitment in response to respiratory viral infection (Ref 25), as well as the inflammatory IL-1 family member, IL18. Expression of a number of chemokines, including ligands for CCR2, were significantly increased in both the lung tissue and airway of COVID-19 patients, including CCL2, CCL3L1, CCL7, CCL8, and CXCL10. Elevated pro-inflammatory IL-1 family members, ILIA and IL1B, were also observed in these 2 compartments. Furthermore, lung tissue exhibited enrichment of the IL-1 cytokine gene signature, whereas the airway exhibited additional expression of IL18, IL33, IL36B, and IL36G.

It was observed that non-hematopoietic cells in the BAL fluid may be indicative of viral-induced damage, as follows. To determine whether viral infection resulted in modification of resident tissue populations, GSVA was performed with various non-hematopoietic cell gene signatures (FIG. 19C). It was observed that signatures of various lung tissue cells but not endothelial cells were enriched in the airway, but not the lung of COVID-19 subjects. Additionally, increased expression was detected of the viral entry genes ACE2 and TMPRSS2, which are typically expressed on lung epithelium (Ref. 26) (FIG. 19D).

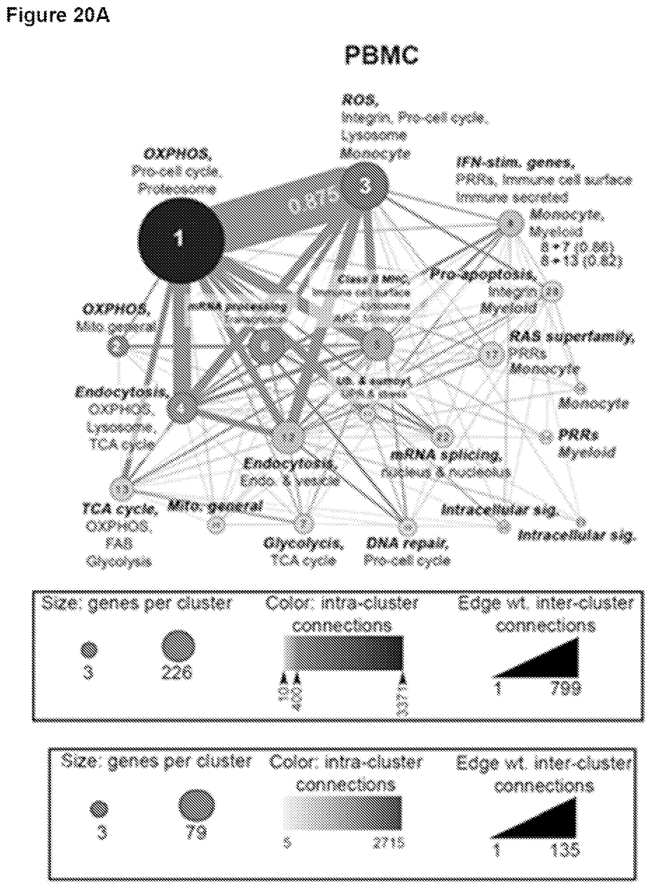

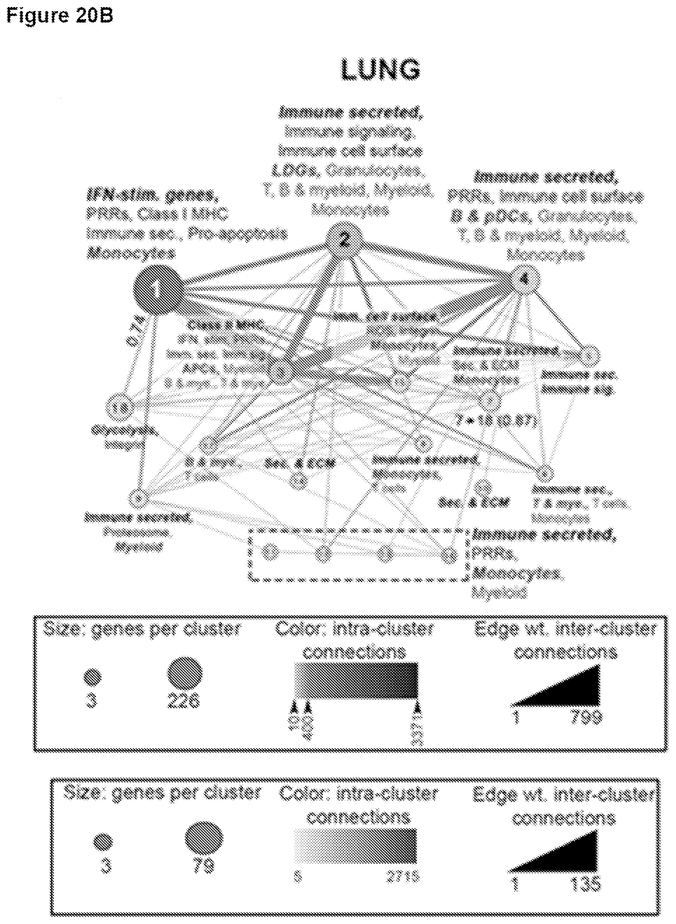

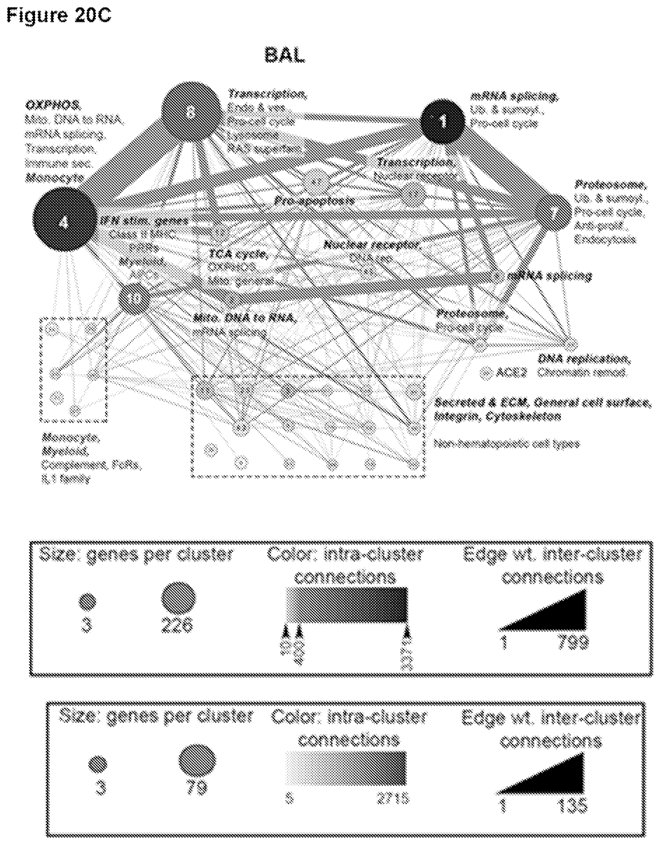

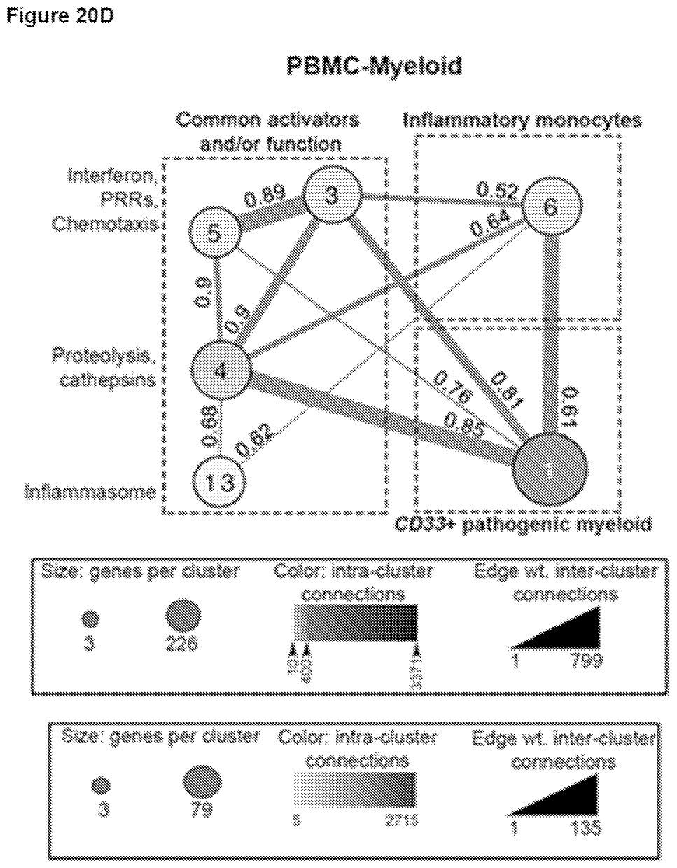

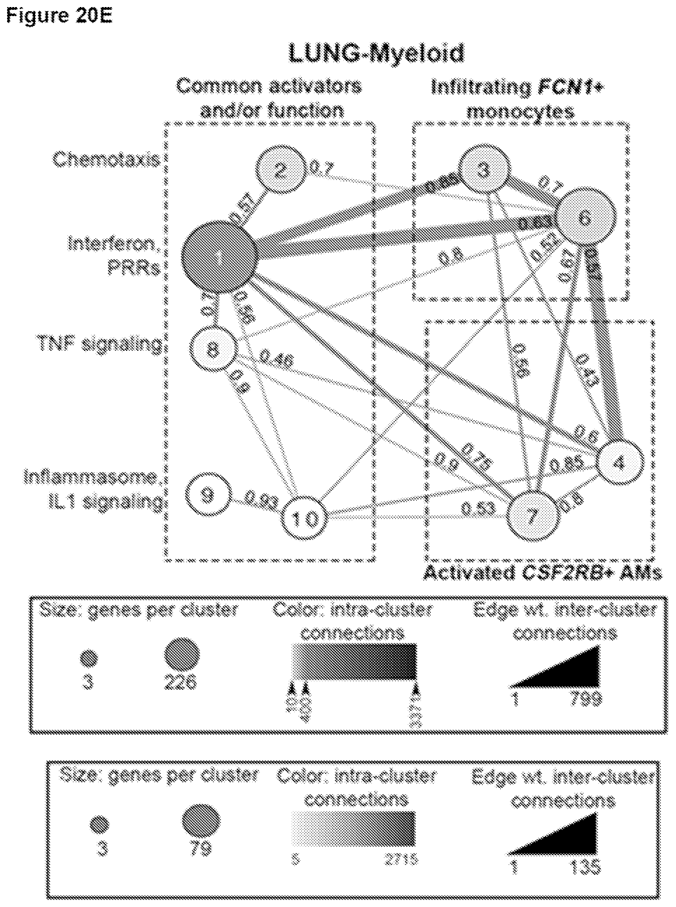

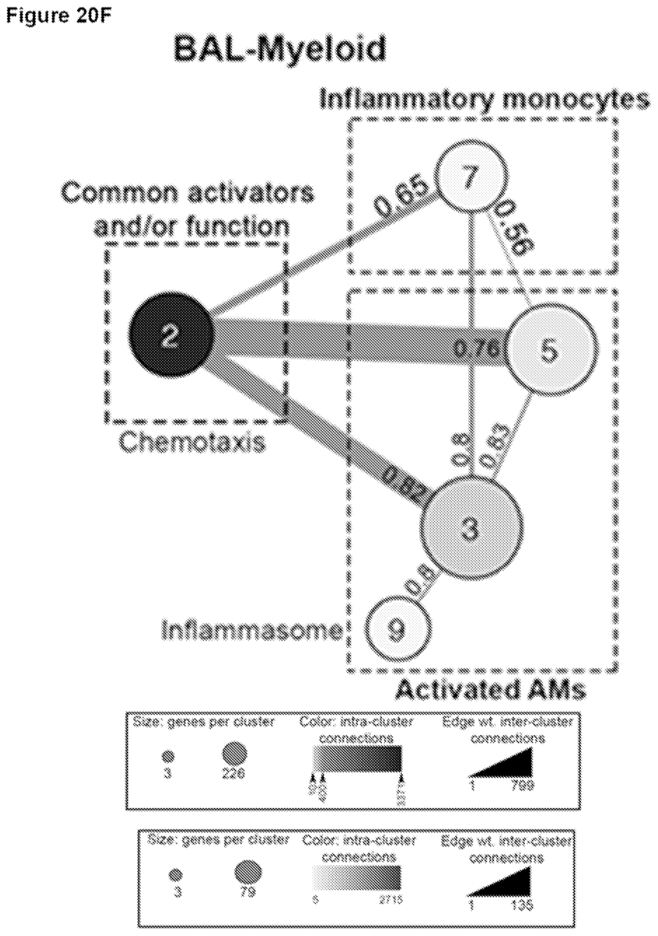

It was observed that protein-protein interactions identify myeloid subsets in COVID-19 patients, as follows. An unbiased, protein-protein interaction (PPI)-based clustering approach was utilized to assess the inflammatory cell types within each tissue compartment. PPI networks predicted from DEGs were simplified into metastructures defined by the number of genes in each cluster, the number of significant intra-cluster connections, and the number of associations connecting members of different clusters to each other (FIG. 20A-20C). Overall, upregulated PPI networks identified numerous specific cell types and functions. In the blood, cluster 8 was dominated by a Mo population expressing C2, C5, CXCL10, CCR2, and multiple IFN-stimulated genes, whereas cluster 3 contained hallmarks of alternatively activated (M2) MΦs and/or myeloid-derived suppressor cells (MDSCs), including CD33, CD36, CD93, and ITGAM (FIG. 20A). Smaller immune clusters were indicative of functions, including inflammasome activation, damage-associated molecular pattern (DAMP) activity, the classical complement cascade and the response to Type II IFNs. Myeloid heterogeneity in the blood was also reflected by the presence of multiple metabolic pathways, such as enhanced oxidative phosphorylation (OXPHOS) in cluster 1 linked to M2-like MΦs in cluster 3 (mean interaction score of 0.875), and glycolysis in clusters 7 and 13 connected to activated Mo in cluster 8 (interaction scores of 0.86 and 0.82, respectively). Consistent with our GSVA results, blood exhibited profoundly decreased T cells determined by the downregulation of T cell activation markers CD28, LCK and ITK (FIG. 17, section a).

In addition to the various Mo/myeloid populations, lung tissue was infiltrated by LDGs, granulocytes, T cells, and B cells. Metabolic function in the lung was varied, with Mo-enriched clusters (1 and 7) linked to glycolysis in cluster 18 (interaction scores of 0.74 and 0.87, respectively) potentially reflecting cellular activation, whereas OXPHOS was predominantly downregulated along with other nuclear processes (transcription and mRNA processing) (FIG. 20B; FIG. 17, section b). The airway was enriched in inflammatory Mo, mitochondrial function and transcription. Multifunctional cluster 4 was dominated by numerous chemokine and cytokine receptor-ligand pairs, whereas smaller immune clusters were enriched in classical complement activation, IFNG and IL-1 responses. (FIG. 20C). Consistent with tissue damage, numerous small clusters were observed in the airway, reflecting the presence of non-hematopoietic cells, including those containing multiple intermediate filament keratin genes, cell-cell adhesion claudin genes and surfactant genes. Notably, non-hematopoietic cell signatures in the airway were similar in content to those derived from in vitro SARS-CoV-2-infected primary lung epithelial cell lines (NHBE) (Ref. 12) (FIG. 17, section d).

Given the large number of clusters including Mo/myeloid/MΦ, next these clusters were examined in greater detail by altering the stringency of PPI clustering to further characterize unique myeloid lineage cells within each tissue compartment (FIG. 20D-20F). Myeloid lineage-specific clusters were then compared to pre-determined gene signatures, including populations G1-G4 reported in BAL of COVID-19 patients (Ref. 27) (FIG. 18A). In the blood, gene modules representative of common myeloid function (chemotaxis, proteolysis, etc.), as well as two independent Mo/myeloid subpopulations (FIG. 20D), were detected. Cluster 6 contained numerous markers highly reminiscent of classically activated blood Mo and exhibited significant overlap with the inflammatory G1 population, whereas cluster 1 was similar to IFN-activated MΦs, CX3CR1+ synovial lining MΦs (from arthritic mice) and alveolar MΦs (AM) (FIG. 18A).

In the lung (FIG. 20E), clusters 2, 3, and 6 overlapped with the G1 inflammatory Mo population and expressed a number of chemotaxis genes. A second population characteristic of AMs was also evident in the lung, defined by CSF2RB, the receptor for GM-CSF, a cytokine that regulates AM differentiation (Refs. 8 and 28-29). Further characterization of this population indicated significant expression of the coagulation system genes F5, FGG, FGL1, SERPINA, and SERPINE2. Similarly, re-clustering of Mo/MΦs/myeloid clusters from the airway revealed a population with hallmarks of inflammatory/M1 MΦ (MARCO and multiple members of the complement cascade; cluster 7), and a second population of AMs (FIG. 20F) demonstrating significant overlap with the G3 and G4 populations (FIG. 18A) (Ref. 27).

Characterization of myeloid populations in COVID-19 patients was performed as follows. The overlap between characterized BAL-defined gene signatures from COVID-19 patients (Ref. 27) and tissue-defined PPI clusters motivated an evaluation of these populations in greater detail by GSVA. Consistent with PPI clusters, the inflammatory-MΦ G1 population was increased in the blood (FIG. 18B). The G1 and G1 & G2 populations were increased in the lung, consistent with the expression of IFN and pro-inflammatory cytokines (FIG. 18C). In the airway, the G2, G3, and G4 populations were significantly enriched indicating the presence of both pro-inflammatory MΦs and AMs (FIGS. 18A-18C). As a whole, it was found that gene signatures of defined Mo/MΦ populations in COVID-19 BAL were dispersed among the blood, lung, and airway compartment.

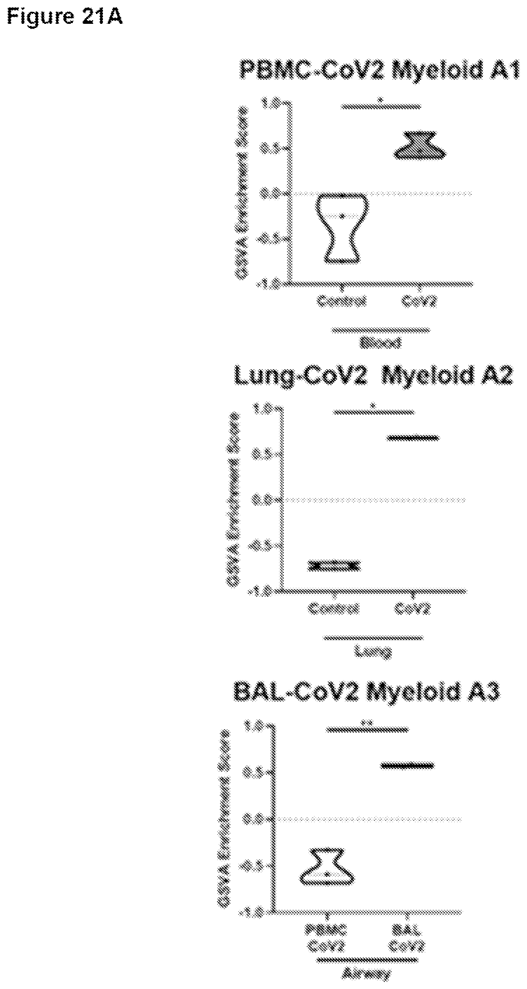

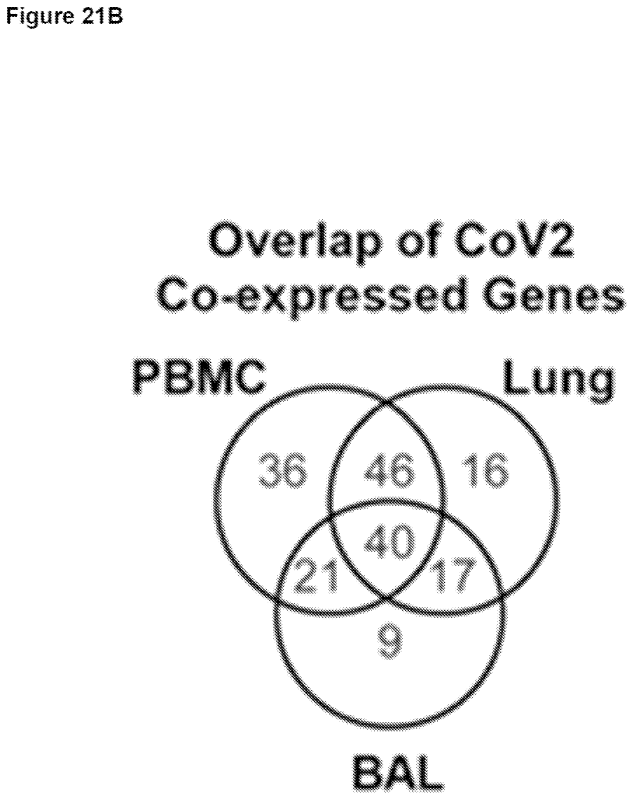

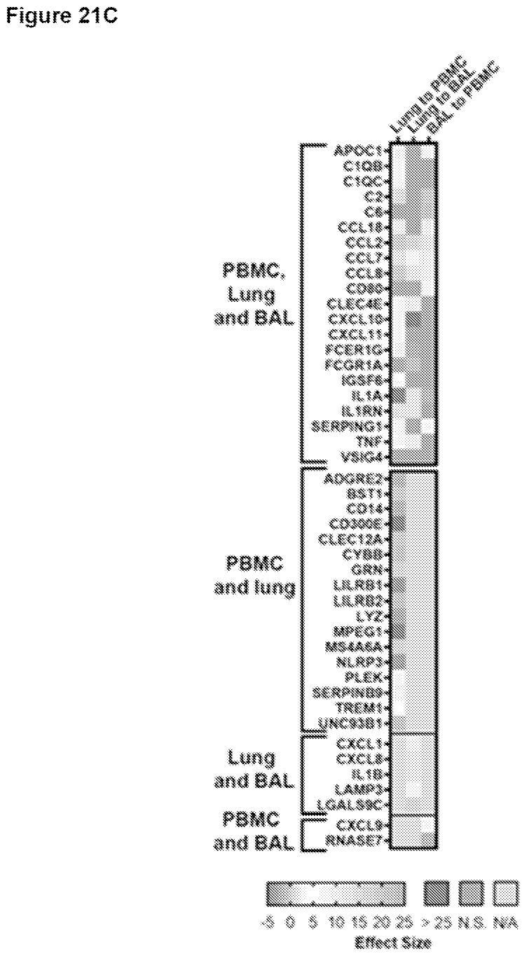

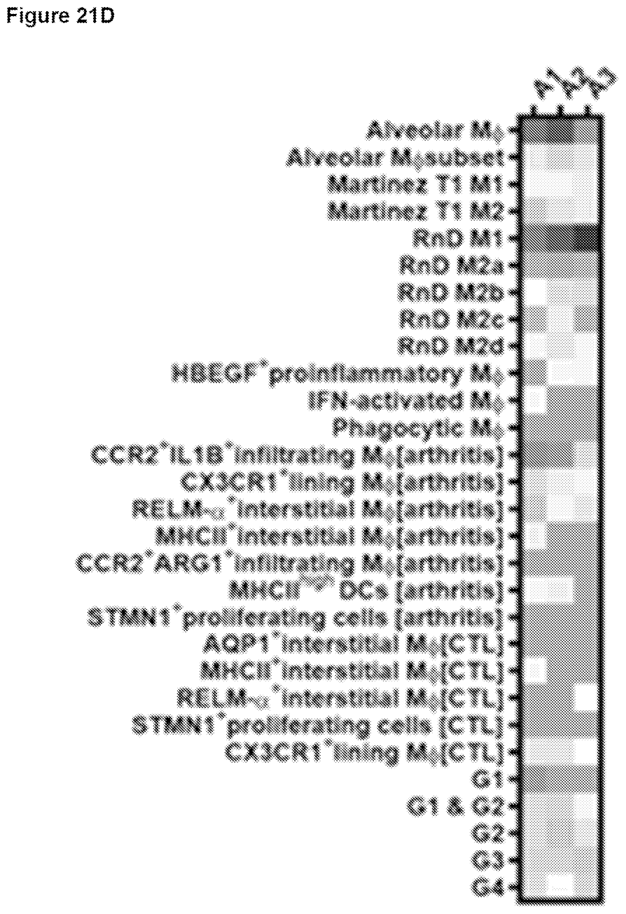

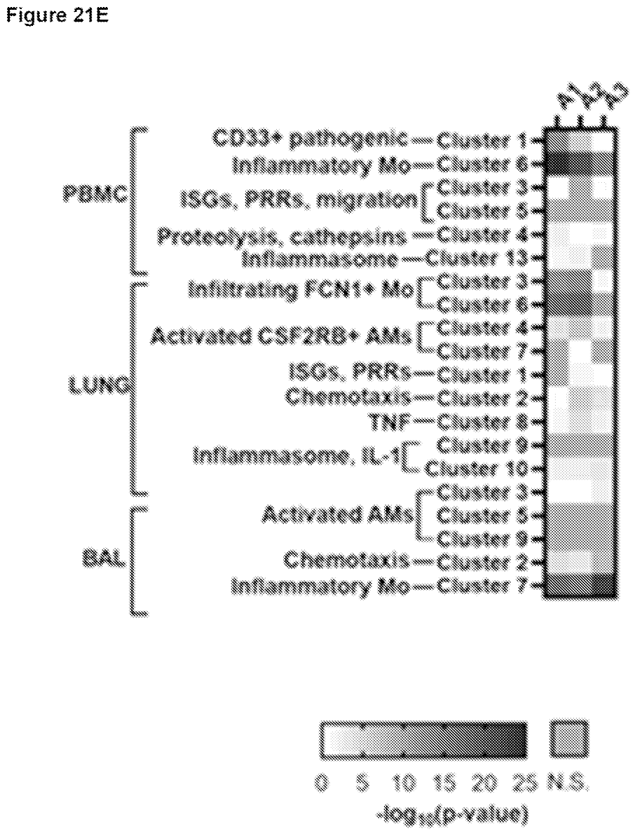

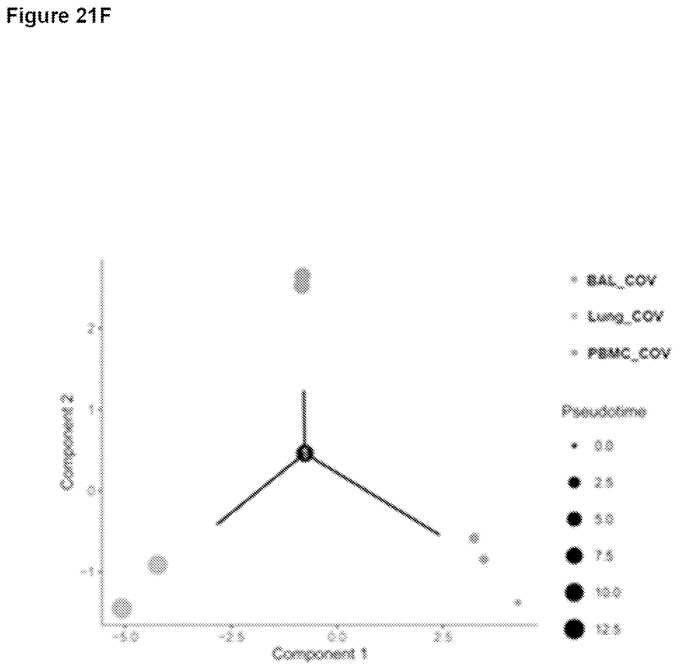

It was observed that co-expression further delineates Mo/MΦ gene expression profiles of COVID-19 patients, as follows. Next, the biology of the populations of Mo/MΦ in the tissue compartments were examined in greater detail. A set of 196 co-expressed Mo/myeloid genes was derived and used (FIG. 19, Tables 7A-7C) to probe heterogeneity in each tissue compartment (FIG. 21A). Notably, co-expression of 40 core genes was observed between all compartments, which included complement, chemokine, and cytokine genes (FIG. 21B). In addition, there were 86 shared co-expressed genes in lung and blood, 57 in the lung and airway, and 61 in the airway and blood (FIG. 21B). To directly compare levels of these 196 co-expressed myeloid genes in each compartment, gene expression was normalized in each sample using 3 genes included in the core 40 genes, (FCGR1A, FCGR2A, FCGR2C) (FIG. 21C). Although many genes were not significantly different between compartments, numerous chemokines and cell surface markers (CCL2, CCL7, CCL8, CXCL10, CLEC4E, FCER1G) and inflammatory cytokines (IL1A and TNF) were enriched in the lung compared to the blood and airway. Furthermore, the complement genes C1QB, C1QC, and C2 were increased in the lung compared to the blood, but not changed between the lung and the airway. Altogether, these normalized gene expression results indicated that expression of inflammatory mediators was increased in SARS-CoV2-infected lung over the other compartments and in the airway compared to the blood.

To determine the function and nature of these myeloid populations, they were compared to other myeloid signatures (FIG. 21D) (Refs. 27 and 30-33). The population increased in the blood (A1) was predominantly characterized by features of AMs, M1 and M2 MΦs, pro-inflammatory MΦs with potential to infiltrate tissue, and the inflammatory MΦ G1 population. The A1 population also exhibited features of inflamed murine residential, interstitial MΦs. The myeloid cell population increased in COVID lung (A2) was most similar to pro-fibrotic AMs, M1 MΦs, M2 MΦs, blood-derived infiltrating MΦs, and the inflammatory Mo G1 population. A2 was also marked by additional AM-specific genes, contributing to the observed overlap with the other two compartments. However, overlap between A2 and the G4 AM signature was relatively decreased, suggesting that the lung AMs are more similar to those found in pulmonary fibrosis (Ref 30). Finally, the population increased in the airway (A3) similarly exhibited characteristics of AMs, M1 and M2 MΦs, and pro-inflammatory MΦs that have infiltrated into the tissue compartment (FIG. 21D). Of note, the airway A3 population was not similar to the BAL-derived inflammatory MΦ G1 population (Ref. 27).

Also, the overlap between the Mo/MΦ A1-A3 gene clusters and those identified using PPI clustering (FIGS. 20A-20F; FIG. 21E) was evaluated. Interestingly, the CD33+ pathogenic population (PPI-derived PBMC Myeloid Cluster 1) was most strongly enriched in the blood, but was also increased in the other compartments. All compartments were characterized by strong enrichment of pro-inflammatory Mo (PBMC Myeloid Cluster 6, Lung Myeloid Clusters 3 and 6, and BAL Myeloid Cluster 7), although A3 exhibited some differences in these populations compared to A1 and A2. Additionally, this comparison suggested enrichment of AMs in all three compartments; however, upon examination of the specific overlapping gene transcripts, the observed enrichment in blood A1 was primarily related to the presence of non-AM-specific myeloid genes. Finally, numerous common activators and functions of PPI-derived clusters were enriched uniformly across A1, A2, and A3, providing further evidence for pro-inflammatory activity of myeloid cell populations in COVID-19 blood and tissue compartments.

Trajectory analysis was performed to understand potential transitions of Mo/MΦ in various tissue compartments. This was based on the normalized 196 myeloid-cell specific genes, as well as 425 additional normalized genes that may be important in Mo/myeloid/MΦ cell differentiation, reflective of chemotaxis, IFN, and metabolism genes. This analysis suggested a branch point of differentiation of Mo/MΦ between blood and lung, with some blood Mo/MΦ differentiating directly to airway cells and others to lung cells in a more protracted manner as indicated by pseudotime (FIG. 21F).

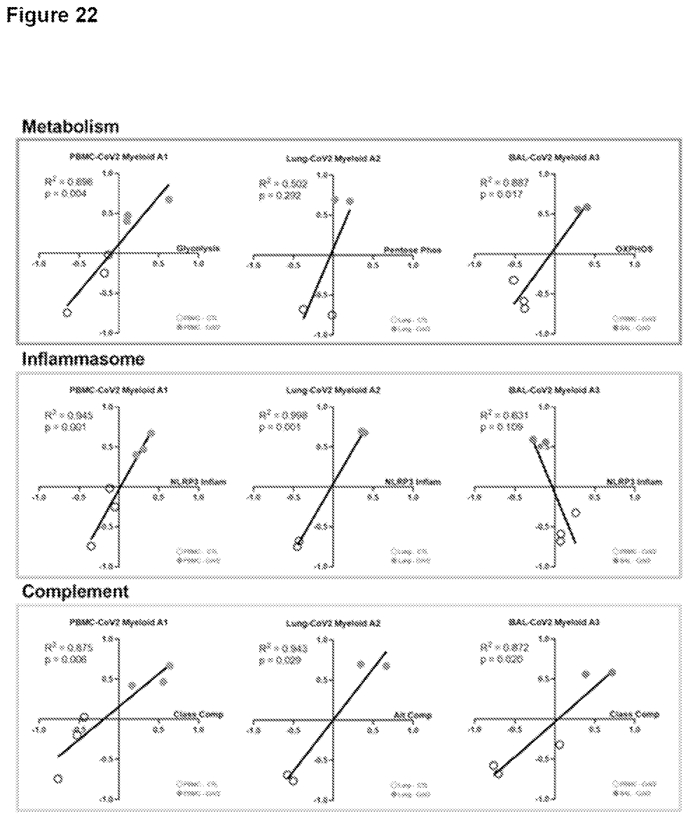

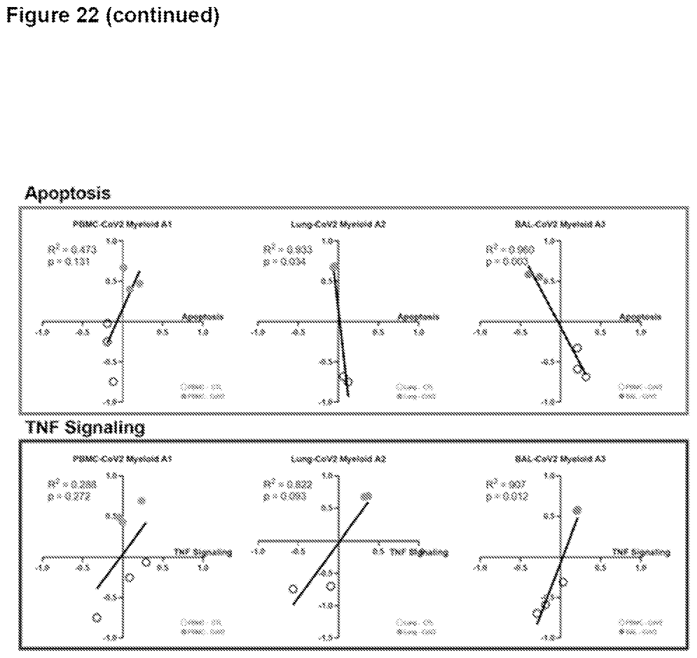

Analysis of the biologic activities of myeloid subpopulations was performed as follows. To focus on functional distinctions among the co-expressed myeloid populations in the blood, lung, and airway compartments (A1-A3), linear regression analyses were utilized between GSVA scores for A1-A3 and scores for metabolic, functional, and signaling pathways (FIG. 22; FIGS. 20A-20F). Blood A1 was significantly correlated with glycolysis, the NLRP3 inflammasome, and the classical and lectin-induced complement pathways. In lung A2, there were no significant correlations detected with metabolism, but this population was significantly correlated with the NLRP3 inflammasome and the alternative complement pathway. Finally, airway A3 was positively correlated with OXPHOS, the classical complement pathway, and TNF signaling and negatively correlated with apoptosis. Overall, these results delineated the heterogeneity in metabolic and inflammatory pathways among myeloid cells enriched in the blood, lung, and airway of COVID-19 patients.

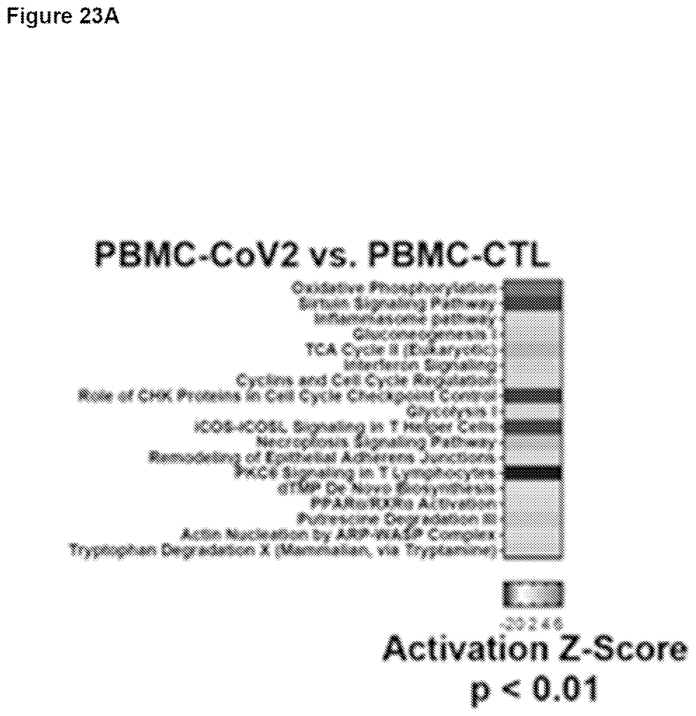

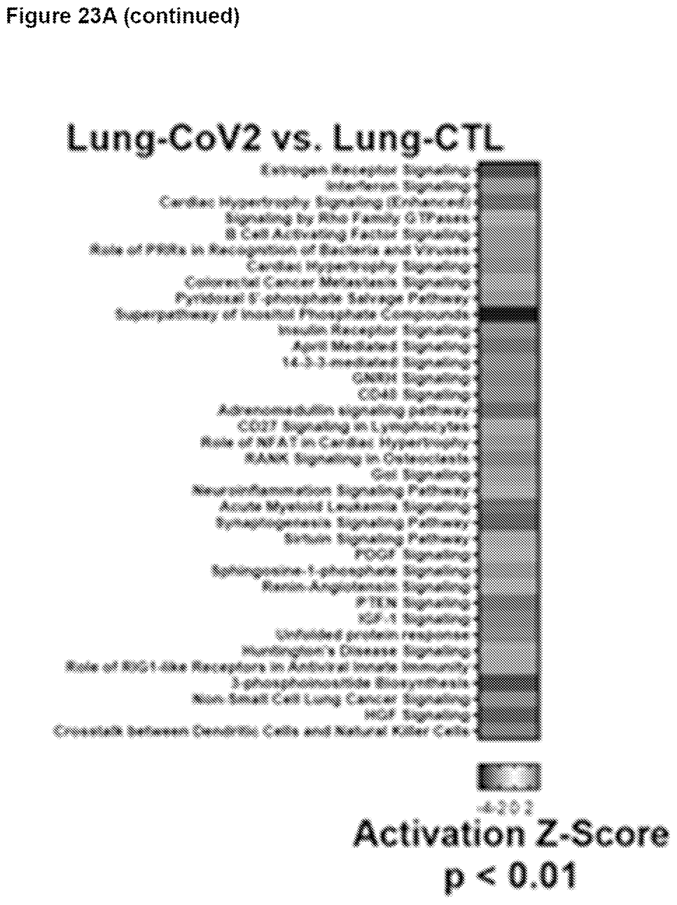

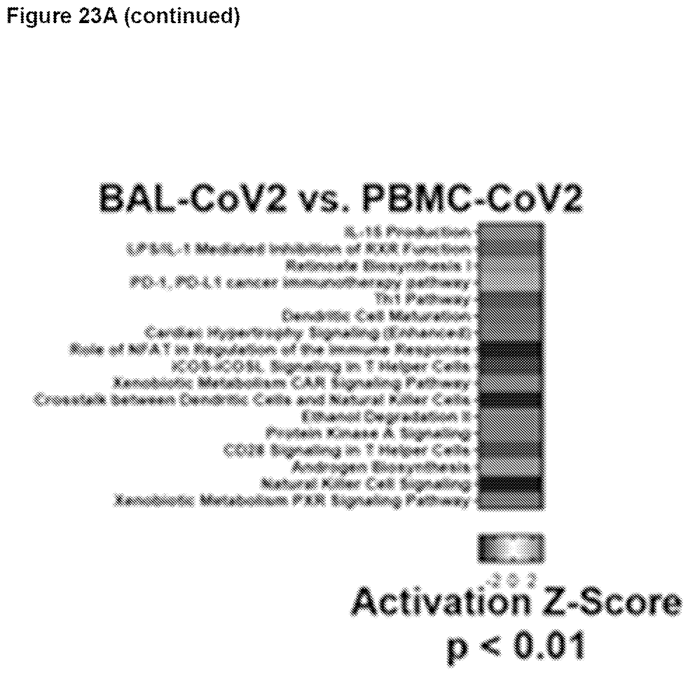

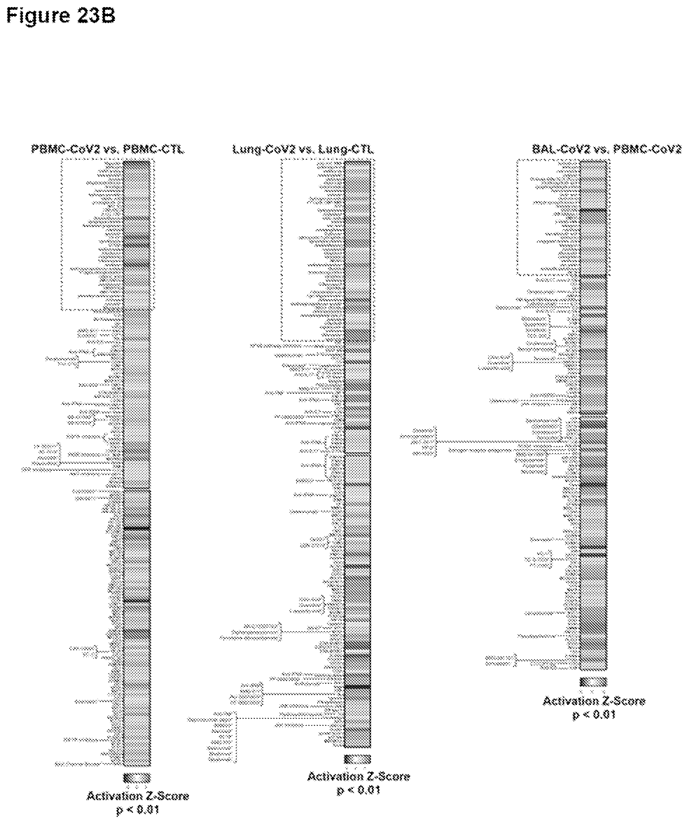

It was observed that pathway and upstream regulator analysis inform tissue-specific drug discovery for treatment of COVID-19, as follows. To understand the biology of SARS-CoV2-infected patients in greater detail, pathway analysis was conducted on DEGs from the 3 compartments using IPA canonical signaling pathway and upstream regulator (UPR) analysis functions (FIGS. 23A-23B). In general, IFN signaling, the inflammasome, and other components of anti-viral, innate immunity were reflected by disease state gene expression profiles compared to healthy controls (FIG. 23A). In addition, metabolic pathways including OXPHOS and glycolysis were significantly increased in the blood of COVID-19 patients compared to controls.

UPRs predicted to drive the responses in each compartment indicated uniform involvement of inflammatory cytokines, with Type I IFN regulation dominant in the SARS-CoV2-infected lung (FIG. 23B). Notable UPRs of COVID-19 blood included IFNA, IFNG, multiple growth factors and ligands, HIF1A, CSF1 and CSF2. Evidence of inflammatory cytokine signaling by IL17 and IL36A was predicted in COVID-19 lung and airway compartments. Whereas the airway DEG profile indicated regulation by both inflammatory and inhibitory cytokines, the COVID-19 lung UPRs were markedly inflammatory, including, NFκB, IL12, TNF, IL1B, and multiple Type I IFNs. These proinflammatory drivers were consistent in each individual lung which were analyzed separately because of the apparent heterogeneity between the lung samples (FIGS. 21A-21F).

IPA analysis was also employed to predict drugs that might interfere with COVID-19 inflammation (FIG. 23B, Tables 8A-8B). Of note, neutralizers of IL17, IL6, IL1, IFNA, IFNG, and TNF were predicted as antagonists of COVID-19 biology. Corticosteroids were predicted to revert the gene expression profile in the SARS-CoV-2-infected lung, but were predicted as UPRs of COVID-19 blood, which may indicate that the patients from whom blood was collected had been treated with corticosteroids rather than indicating that these agents were driving disease pathology. Chloroquine (CQ) and hydroxychloroquine (HCQ) were additionally predicted to revert the COVID-19 transcription profile in the lung, which may point to their potential utility as treatment options. A number of drugs matched to unique targetable pathways in the lung, including NFκB pathway inhibitors and neutralizers of the TNF family; however, some drugs also targeted pathways shared by both the lung and airway, including JAK inhibitors. In the BAL-CoV2 vs. PBMC-CoV2 IPA comparison, several drugs were matched to UPRs with a negative Z-score, which provided additional therapeutic options directed towards the blood of patients with COVID-19, given that molecules targeting downregulated or inhibited UPRs are molecules that could normalize the PBMC-CoV2 gene signature. As such, several possible therapeutics arose from this analysis to target COVID-19 blood, including ustekinumab, targeting the IL12/23 signaling pathway, and lenalidomide, which have immunosuppressive effects. In addition, IGF1R inhibitors, EGFR inhibitors, VEGFR inhibitors, and AKT inhibitors were among the compounds predicted to target COVID-19 PBMCs. No specific drugs were predicted to target all three tissue compartments, but each compartment was driven by inflammatory cytokines.

Another approach to predict possible drug targets is by employing connectivity scoring with drug-related gene expression profiles using the perturbagen CMAP database within CLUE (Tables 8A-8B). Although CLUE-predicted drugs tended to differ from those predicted by IPA or those matched to IPA-predicted UPRs, there were some overlapping mechanisms, including inhibition of AKT, angiogenesis, CDK, EGFR, FLT3, HSP, JAK, and mTOR. IPA-predicted drugs that were unique from connectivity-predicted drugs tended to capture more cytokine and lymphocyte biology, including inhibitors of IL1, IL6, IL17, TNF, type I and II interferon, CD40LG, CD38, and CD19, among other cytokines and immune cell-specific markers. Overall, the gene expression-based analysis of SARS-CoV2-infected blood and tissue compartments indicated several existing treatment options that may be evaluated as candidates to treat COVID-19, and subsequently administered to patients having COVID-19.

Multiple orthogonal bioinformatics approaches were employed to analyze DEG profiles from the blood, lung, and airway of COVID-19 patients, and revealed the dynamic nature of the inflammatory response to SARS-CoV-2 and possible points of therapeutic intervention. In the blood, evidence was seen of myeloid cell activation, lymphopenia, and elevation of plasma cells, as has been shown by both standard cell counts, flow cytometry and gene expression analysis (Ref 34). In the lungs, increased gene signatures were found of additional myeloid cell types including granulocytes, infiltrating inflammatory Mo, and AMs as well as the presence of non-activated T, B, and NK cells. Furthermore, inflammation in the lung tissue was enhanced by the greater presence of IFNs and more pro-inflammatory cytokines than observed in the blood. Finally, in the airway evidence was found of blood and AM-derived inflammatory and regulatory MΦs, and non-hematopoietic lung tissue cells accompanied by expression of SARS-CoV-2 receptors, and alarmins, indicative of viral infection and damage to the lung and consistent with detection of SARS-CoV2 in BAL fluid (Refs. 11 and 35). Together these findings indicate a systemic, but compartmentalized immune and inflammatory response with specific signs of cellular activation in blood, lung, and airway. This has informed a more comprehensive and integrated model of the nature of the local and systemic host response to SARS-CoV2.

The predominant populations of immune cells found to be enriched and activated in COVID-19 patients were myeloid cells and, in particular, subsets of inflammatory Mo and MΦs, which differed between the blood, lung, and airway compartments. In the peripheral blood, significant enrichment of Mo was found, including classically activated inflammatory M1 MΦs as well as a CD33+ myeloid subset, which appeared to be an M2 population reminiscent of characterized IFN-activated MΦs, AMs, and MDSCs, indicative of a potential regulatory population induced by stimuli arising from the SARS-CoV2-infected lung. Myeloid cells enriched in the blood of COVID-19 patients were also highly correlated with gene signatures of metabolic pathways (Glycolysis, Pentose Phosphate Pathway, and TCA cycle) indicative of pro-inflammatory M1 MΦs (Ref. 36).

The lung tissue was enriched in gene signatures of Mo/MΦs as well as other myeloid cells including two populations of granulocytes, neutrophils and LDGs. Increases in blood neutrophils may be associated with poor disease outcome in COVID-19 patients, and the formation of neutrophil extracellular traps (NETs) may contribute to increased risk of death from SARS-CoV2 infection (Refs. 37-39). In addition, populations of dysregulated neutrophils expressing pro-inflammatory or suppressive markers derived from scRNA-seq of COVID-19 patient PBMCs may be characterized and found to be positively correlated with disease severity (Refs. 18-19). These populations were found to be also increased in SARS-CoV2 infected lung tissue and, therefore, indicate that they may contribute to lung pathology. Although LDGs have not been reported in the COVID-19 lung, in comparison to neutrophils, they exhibit an enhanced capacity to produce Type I IFNs and form NETs and therefore, may have an even greater impact on disease progression (Ref 40).

Mo/MΦ subsets in the lung of COVID-19 patients were characterized as infiltrating inflammatory Mo and activated AMs, which exhibited a mixed metabolic status suggestive of different states of activation. Infiltrating Mo from the peripheral blood appeared to be further activated in the lung tissue as evidenced by enhanced expression of markers of highly inflammatory Mo characterized in severe COVID-19 cases (Ref 27). The AM population enriched in COVID lung tissue clustered with genes involved in the coagulation system, which is consistent with observations of procoagulant AM activity in COVID-19 and in ARDS (Ref. 41). As pulmonary thrombosis has been associated with poor clinical outcomes in COVID-19 patients, this result indicates that activated AMs in SARS-CoV2-infected lung tissue may be involved in facilitating a pro-thrombotic status, and thereby, contribute to poor disease outcome (Ref 42). Finally, a trend was noted toward an increase in platelets specifically in the COVID-19 lung, indicating that they may also contribute to thrombosis in some patients.

In the airway, gene signatures were detected of various post-activated MΦ subsets including inflammatory M1 MΦs, alternatively activated M2 MΦs, and activated AMs. Expression of myeloid cell genes in the airway also correlated with a signature of oxidative metabolism, which is characteristic of M2 macrophages and typically associated with control of tissue damage (Ref. 36). However, in the context of pulmonary infection, polarization of AMs toward an anti-inflammatory M2 phenotype was found to promote continued inflammation, suggesting that these MΦs may not be effective at resolving anti-viral immunity (Ref. 43).

In addition to myeloid cells, inflammatory mediators from the virally infected lung typically promote migration and activation of NK cells and adaptive immune cells including T and B cells (Ref 8). Significant deficiencies were found in gene signatures of T cells and cytotoxic CD8 and NK cells, consistent with clinical evidence of lymphopenia in the peripheral blood and airway of COVID-19 patients (Refs. 44-47). In contrast to T and NK cells, increased evidence was observed of B cell activation through CD40/CD40L and an increased plasma cell signature in the blood of COVID-19 patients. This result indicates that COVID-19 patients are able to mount an antibody-mediated immune response. However, whether a virus-specific antibody response is beneficial to recovery from SARS-CoV2 infection may be unclear (Ref. 48). Low quality, low affinity antibody responses to SARS-CoV may promote lung injury in some patients, although it may be unknown if this occurs in SARS-CoV2 infected individuals (Refs. 49-50).

The contents of the airway as assessed through the BAL fluid, act as a window into events in the alveoli and airways and can be used to understand what is happening in the infected tissue that is separate from the interstitium of the lung (Refs. 51-52). Increased enrichment was found of lung epithelial cells in the airway of COVID-19 patients, indicating that SARS-CoV2 infection of alveolar cells together with localized inflammation as a result of enhanced myeloid cell infiltration promote significant damage to the alveoli and result in affected cells being sloughed into the airway. Furthermore, the lack of cytotoxic cells and thus, the inability to clear these virus-infected lung epithelial cells in the airway likely accounts for the increased presence of post-activated MΦs and high expression of pro-inflammatory IL-1 family members observed in the BAL of COVID-19 patients. Importantly, these results indicate that sampling of BAL may provide an important mechanism to evaluate the impact of SARS-CoV2 infection.

DEGs from COVID-19 patients were enriched in IGS, complement pathways, inflammatory cytokines and the inflammasome, which may be expected to activate Mo/MΦ populations in the blood, lung, and airway of COVID-19 patients and initiate a robust and systemic response to infection. In particular, these results indicate that IL-1 family-mediated inflammation plays a critical role in COVID-19 pathogenesis. However, pro-inflammatory genes identified via GWAS as contributing to COVID-19 inflammation, including CCR2, CCR3, CXCR6, and MTA2B, were not significantly different from controls in the lung dataset (Ref 53). Thus, in contrast to characterizations of both SARS-CoV and SARS-CoV2 infection, these analyses did not indicate the presence of overwhelming pro-inflammatory cytokine production or “cytokine storm” in COVID-19 patients (Refs. 34 and 54). Rather, these data indicates that the increased numbers, overactivation, and potentially heightened pathogenicity of monocyte/MΦ populations are the main drivers of the dysregulated inflammatory response and resulting tissue damage in COVID-19 patients.

In the absence of proven antiviral treatment and/or a SARS-CoV2-specific vaccine, disease management may be reliant upon supportive care and therapeutics capable of limiting the severity of clinical manifestations. Using empiric evidence as a guide, the current approach may be successful in identifying “actionable” points of intervention in an unbiased manner and in spite of formidable patient heterogeneity. Analyses presented here support COVID-19 infection-related increases in inflammatory cytokines, particularly IL6 and TNF, both of which function as predictors of poor prognosis (Refs. 55-56), as well as complement activation (Refs. 37 and 57-58). Accordingly, anti-IL6 therapies including sarilumab, tocilizumab and clazakizumab, as well as biologics targeting terminal components of the complement cascade, such as eculizumab and ravulizumab, are in various clinical trial phases for treating COVID associated pneumonia. Candidate TNF blockers such as adalimumab, etanercept and many others, represent additional options for inhibiting deleterious pro-inflammatory signaling. However, most showed patient heterogeneity, indicating a requirement to identify the specific cytokine profile in each patient in order to offer personalized treatment. Our analyses also indicate the likely involvement of pro-inflammatory IL1 family members especially in the lung, suggesting anti-IL1 family interventions, including canakinumab and anakinra, may be effective in preventing acute lung injury.

This analysis also establishes the predominance of inflammatory Mo/myeloid lineage cells in driving disease pathology and indicates therapies effective at blocking myeloid cell recruitment or forcing repolarization may prevent disease progression. CCL5 (RANTES) is a potent leukocyte chemoattractant that interacts with multiple receptors, including CCR1 (upregulated in the blood, lung and airway), and CCR5 (upregulated in the airway). Disruption of the CCR5-CCL5 axis may be tested using the CCR5 neutralizing monoclonal antibody leronlimab in a compassionate use trial (Ref 59).

It may be observed that COVID-19 may predispose patients to thromboembolic disease (Refs. 60-61). Indeed, the gene expression analyses presented here showing altered expression of coagulation factors and fibrinogen genes indicate dysfunction within the intrinsic clotting pathway. These findings, together with evidence of excessive inflammation, complement activation and the involvement of LDGs in lung inflammation, may contribute to the systemic coagulation underlying the remarkably high incidence of thrombotic complications observed in severely ill patients, thereby reinforcing recommendations to apply pharmacological anti-thrombotic medications.

Further, anti-rheumatic drugs may be used for managing COVID-19. For example, CQ is a compound predicted as a UPR with potential phenotype-reversing properties. In vitro experiments examining the anti-viral properties of CQ and its derivative HCQ were effective in limiting viral load; however, the efficacy of these drugs in clinical trials has been less clear (Ref. 62). Questions about drug timing, dosage and adverse events may call into question the use of these drugs for COVID-19 patients. Despite results showing no negative connectivity between the gene signatures of SARS-CoV2 infection and HCQ treatment (Ref. 35), IPA predicted a role of anti-malarials as limiting the function of intracellular TLRs in the lung and also as a direct negative UPR of gene expression abnormalities in the lung, indicating a role in controlling COVID-19 inflammation and not viral replication. Further clinical testing may be performed to establish this possible utility; subsequently, these anti-malarials may be administered to COVID-19 patients to treat disease.

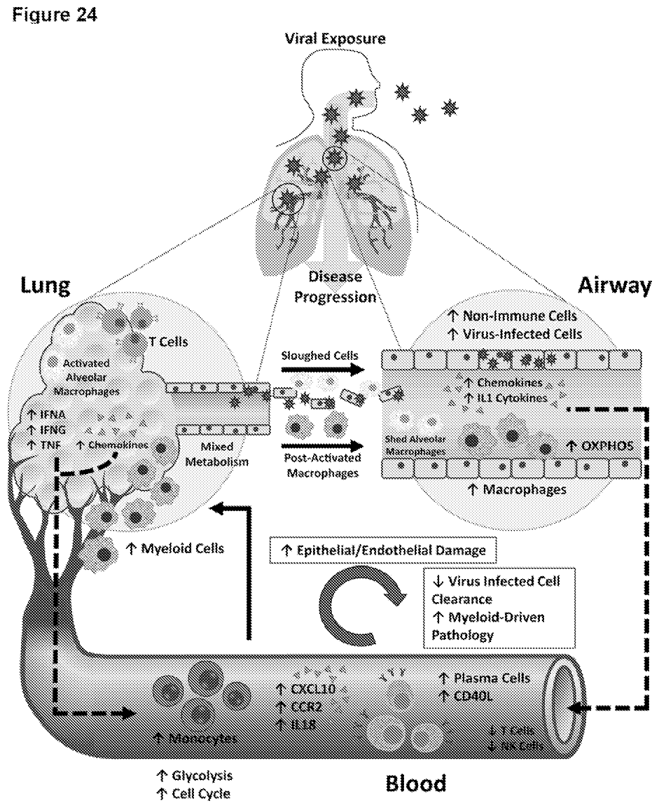

By comparing the transcriptomic profile of the blood, lung, and airway in COVID-19 patients, a model of the systemic pathogenic response to SARS-CoV2 infection has emerged (FIG. 24). SARS-CoV2 infection leads to systemic Mo/MΦ activation, likely as a result of the release of pro-inflammatory mediators from infected cells. Infiltration of immune cells into the lung tissue and alveolus, in particular, neutrophils, LDGs, and pathogenic Mo/MΦ populations promotes a cycle of inflammatory mediator release and further myeloid cell activation, which exacerbates inflammation in the lung and leads to tissue damage. The local release of complement components and clotting factors by infiltrating Mo/MΦ may contribute to both inflammation and thrombotic events. As disease progresses, increased infiltration of pro-inflammatory immune cells, release of inflammatory mediators, and damage to the infected alveolus is reflected by the presence of Mo/MΦ cells and lung epithelial cells in the airway (Ref 51). Furthermore, evidence of both Mo-derived inflammatory MΦs and AMs in the airway compartment indicates that myeloid cell populations from both the blood and the lung tissue are present in the BAL. The accumulation of virus-infected cells and release of alarmins in the airway may not only reflect ongoing infection, but also promote inflammation and prevent resolution of the infection and foster the continuation of the innate immune response. Therefore, sampling the BAL fluid is an effective strategy to monitor tissue inflammation and damage in COVID-19 patients.

As SARS-CoV2 continues to propagate, viral clearance may be impaired by a lack of cytotoxic CD8 T cells and NK cells. This is consistent with MAS occurring in other settings, in which defects in cytotoxic activity of CD8 T cells and NK cells result in enhanced innate immune cell activation and intensified production of pro-inflammatory cytokines, many of which were also expressed in COVID-19 patients (Refs. 9-10). Thus, the results indicated that the lack of activated CD8 T cells and NK cells and subsequent failure to clear virus-infected cells, is a major contributor to the MΦ-driven pathologic response to SARS-CoV2 observed in COVID-19 patients.

In order to develop a model of SARS-CoV2 infection, multiple orthogonal approaches were utilized to analyze gene expression from COVID-19 patients. Heterogeneity among patients in any given cohort may have an increased impact on the overall outcome. One possible reason for this intra-cohort heterogeneity is that the patients may have exhibited varying levels of disease severity. Therefore, additional studies may be performed on more patients, such as by accounting for differences in demographic information and disease status, in order to increase the power of downstream analyses.

As shown, transcriptomic analysis has contributed critical insights into the pathogenesis of COVID-19. Diffuse Mo/MΦ activation is the likely primary driver of clinical pathology. Therefore, this work provides a rationale for placing greater focus on the detrimental effects of exaggerated activation of pathogenic Mo/MΦs and for targeting these populations as an effective treatment strategy for COVID-19 patients.

Ethics statement: publicly available data sets used in this study are listed in Table 5. For each dataset, all patient samples were collected in adherence to local regulations and after obtaining institutional review board approved informed consent. The study corresponding to accession number CRA002309 was approved by the Ethics Committee of the Zhongnan Hospital of Wuhan University. The study corresponding to accession number GSE147505 was approved by the institutional review board at the Icahn School of Medicine at Mount Sinai under protocol HS #12-00145.

Read quality, trimming, mapping and summarization were performed as follows. RNA-seq data were processed using a consistent workflow using FASTQC, Trimmomatic, STAR, Sambamba, and featureCounts. As described below SRA files were downloaded and converted into FASTQ format using SRA toolkit. Read ends and adapters were trimmed with Trimmomatic (version 0.38) using a sliding window, ilmnclip, and headcrop filters. Both datasets were head cropped at 6 bp and adapters were removed before read alignment. Reads were mapped to the human reference genome hg38 using STAR, and the .sam files were converted to sorted .bam files using Sambamba. Read counts were summarized using the featureCounts function of the Subread package (version 1.61).

The RNA-seq tools are all free, open source programs, as follows: SRA toolkit (github.com/ncbi/sra-tools); FastQC (www.bioinformatics.babraham.ac.uk/projects/fastqc/); Trimmomatic (www.usadellab.org/cms/?page=trimmomatic); STAR (github.com/alexdobin/STAR); labshare.cshl.edu/shares/gingeraslab/www-data/dobin/STAR/STAR.posix/doc/STARmanual.pdf; Sambamba (lomereiter.github.io/sambamba/); and FeatureCounts (subread.sourceforge.net).

Differential gene expression and gene set enrichment analysis were performed as follows. The DESeq2 workflow was used for differential expression analysis. Comparisons were made between control PBMCs and PBMCs from COVID-19 patients (PBMC-CTL vs. PBMC-CoV2) and control lung tissue and lung tissue from COVID-19 patients (Lung-CTL vs. Lung-CoV2). Since no corresponding control BAL samples were available for the COVID-19 BAL samples, BAL samples were compared from COVID-19 patients to COVID-19 PBMC (PBMC-CoV2 vs BAL-CoV2). This was possible because these samples were analyzed on the same platform, run at the same time. Also, normal BAL were compared to BAL of asthmatic individuals to identify genes unrelated to COVID-19 (PRJNA434133).

Two technical replicates were included for BAL cohort, and 4 technical replicates were included for postmortem lung samples. The replicates were collapsed and averaged into one using collapsereplicates function from DESeq2 package. The genes with low expression (i.e genes with very few reads) were removed by filtration. The filtered raw counts were normalized using the DESeq method and differentially expressed genes were determined by FDR<0.2 (Ref 63). Counts were then log 2 transformed and used for downstream analyses (Table 6).

Gene Set Variation Analysis (GSVA) was performed as follows. The GSVA (version 1.25.0) software package (Ref. 64) is an open source package available from R/Bioconductor and was used as a non-parametric, unsupervised method for estimating the variation of pre-defined gene sets in patient and control samples of microarray and RNA-seq expression data sets (www.bioconductor.org/packages/release/bioc/html/GSVA.html). The inputs for the GSVA algorithm were a gene expression matrix of log 2 expression values for pre-defined gene sets. All genes within a gene set were evaluated if the interquartile range (IQR) of their expression across the samples was greater than 0. Enrichment scores (GSVA scores) were calculated non-parametrically using a Kolmogorov Smirnoff (KS)-like random walk statistic and a negative value for a particular sample and gene set, indicating that the gene set has a lower expression than the same gene set with a positive value. The enrichment scores (ES) were the largest positive and negative random walk deviations from zero, respectively, for a particular sample and gene set. The positive and negative ES for a particular gene set depend on the expression levels of the genes that form the pre-defined gene set. GSVA calculates enrichment scores using the log 2 expression values for a group of genes in each SARS-CoV2 patient and healthy control and normalizes these scores between −1 (no enrichment) and +1 (enriched). Welch's t-test was used to calculate the significance of the gene sets between the cohorts. Significant enrichment of gene sets was determined by p-value <0.05.

Derivation of GSVA Gene Sets was performed as follows. Cellular and inflammatory modules and IFN-induced gene sets were derived (Refs. 14-15). Additional inflammatory pathways (NLRP3 Inflammasome, Classical Complement, Alternative Complement, and Lectin-induced Complement were curated from the Molecular Signatures Database. Other signatures were derived (Refs. 27, 30-33, and 65).

Additional hematopoietic cellular gene signatures (monocyte, myeloid, and neutrophil) were derived from I-Scope, a tool developed to identify immune cell specific genes in big data gene expression analyses. Non-hematopoietic fibroblast and lung cell gene sets were derived from T-Scope, a tool developed to identify genes specific for 45 non-hematopoietic cell types or tissues in big gene expression datasets. The T-Scope database contains 1,234 transcripts derived initially from 10,000 tissue enriched and 8,000 cell line enriched genes listed in the Human Protein Atlas. From the list of 18,000 potential tissue or cell specific genes, housekeeping genes and genes differentially expressed in 40 hematopoietic cell datasets were removed. The final gene lists were checked against available single cell analyses to confirm cellular specificity.

Nine single-cell RNA-seq lung cell populations (AT1, AT2, Ciliated, Club, Endothelial, Fibroblasts, Immuno Monocytes, Immuno T Cells, and Lymphatic Endothelium) were downloaded from the Eils Lung Tissues set (Ref. 66) accessed by the UC Santa Cruz Genome Browser (eils-lung.cells.ucsc.edu). Genes occurring in more than one cell type were removed. Additionally, genes known to be expressed by immune cells were removed. The Eils Lung Tissues set Immuno Monocyte, Immuno T Cell, Fibroblast, and Lymphatic Endothelium categories were not employed in further analyses.

Apoptosis and NFkB gene signatures were derived and modified from Ingenuity Pathway Analysis pathways Apoptosis Signaling and NFkB Signaling. ROS-protection was derived from Biologically Informed Gene-Clustering (BIG-C).

Network analysis and visualization were performed as follows. Visualization of protein-protein interaction and relationships between genes within datasets was done using Cytoscape (version 3.6.1) software. STRING (version 1.3.2) generated networks were imported into Cytoscape (version 3.6.1) and partitioned with MCODE via the clusterMaker2 (version 1.2.1) plugin. For PPIs in FIGS. 20A-20C, STRING settings were adjusted to high confidence (0.7), for PPIs in FIGS. 20D-20F, settings were relaxed to medium confidence (0.4). All PPIs were generated without the neighborhood or textmining features. For some PPIs, the average interaction strength using STRING-based cumulative interaction scores was used to determine the strength of interaction between clusters.

Functional and cellular enrichment analysis was performed as follows. Functional enrichment of clusters was performed using Biologically Informed Gene-Clustering (BIG-C), which was developed to understand the potential biological meaning of large lists of genes (Ref 67). Genes are clustered into 53 categories based on their most likely biological function and/or cellular localization based on information from multiple on-line tools and databases including UniProtKB/Swiss-Prot, GO terms, KEGG Pathways, MGI database, NCBI PubMed, and the Interactome. Hematopoietic cellular enrichment was performed using I-Scope, a tool developed to identify immune cell specific genes in big data gene expression analyses. Statistically significant enriched types of cell types in DEGs were determined by Fisher's Exact test overlap p-value and then determining an Odds Ratio of enrichment.

Derivation of co-expressed myeloid subpopulations in each compartment was performed as follows. Co-expression analyses were conducted in R. Sample (control and patient) log 2 expression values for each gene of the 221 identified monocyte/myeloid cell genes in were analyzed for their Pearson correlation coefficient in each tissue compartment (blood, lung, and airway) using the Cor function. Of note, only 196 of 221 genes had changes in gene expression in at least one tissue by RNA-seq. Pearson correlations for these 196 genes were hierarchically clustered by their Euclidian distance into 2 clusters (k=2) using the heatmap.2 function in R. This resulted in 2 Mo/myeloid co-expressed clusters in each compartment corresponding to increased and decreased gene sets. The upregulated co-expressed genes were used to define the A1, A2, and A3 myeloid subpopulations from the blood, lung, and airway compartments, respectively (Tables 7A-7C). The co-expressed myeloid populations in each compartment (A1-A3) were then evaluated for enrichment by GSVA.

Inter-compartment myeloid gene comparisons were performed as follows. To compare relative expression of the 196 myeloid-specific genes among compartments, HTS filtered log 2 expression values for each gene were normalized to the average expression of FCGR1A, FCGR2A, and FCGR2C in each sample. Welch's t-test was used to calculate the significant differences in normalized gene expression between cohorts. Effect sizes were computed between cohorts using the cohen.d function with Hedges' correction in R.

Monocle analyses were performed as follows. Trajectory analyses were performed with Monocle (Refs. 68-70) version 2.14.0 in R. Gene expression values for 621 genes related to myeloid cell differentiation and function including cell surface and secreted markers, M1 and M2 markers, metabolism, and IFN genes were selected as a curated input list (Tables 7A-7C). The HTS filtered log 2 expression values for each of these genes in each sample for each tissue type (PBMC-CoV2, Lung-CoV2, and BAL-CoV2) was normalized by the average log 2 expression of FCGR1A, FCGR2A, and FCGR2C in that particular sample as described above. Normalized expression of these genes was used as the input expression data for Monocle. The CellDataSet was created with parameters of lowerDetectionLimit=0.01 and expressionFamily=uninormal( ). Dimensions were reduced using the DDRTree method, and the max_components parameter was set to 2. Cell state was ordered based upon the state corresponding to PBMC-CoV2.

Linear Regression Analysis was performed as follows. Simple linear regression between calculated myeloid subpopulation A1, A2, and A3 GSVA scores and biological functions or signaling pathway GSVA scores was performed in GraphPad Prism Version 8.4.2. In all analyses where pathway genes (e.g. classical complement) were also myeloid cell genes, these genes were removed from the myeloid GSVA score for that comparison and kept in the pathway GSVA score. For each regression analysis, the Goodness of Fit is displayed as the R squared value and the p-value testing the significant of the slope is displayed. All p-values are displayed with 3 digits and rounded-up unless rounding changes significance.

Ingenuity Pathway Analysis (IPA) was performed as follows. The canonical pathway and upstream regulator functions of IPA core expression analysis tool (Qiagen) were used to interrogate DEG lists. Canonical pathways and upstream regulators were considered significant if |Activation Z-Score|≥2 and overlap p-value <0.01. Chemical reagents, chemical toxicants, and endogenous non-mammalian ligands were culled from all upstream regulator analyses.

Drug-Target Matching was performed as follows. IPA-predicted upstream regulators were annotated with respective targeting drugs and compounds to elucidate potential useful therapies in SARS-CoV2. Drugs targeting gene products of interest by both direct and indirect targeting mechanisms were sourced by Combined Lupus Treatment Scoring (CoLTS)-scored drugs (Ref 71), the Connectivity Map via the drug repurposing tool, DrugBank, and literature mining. Similar methods were employed to determine information about drugs and compounds, including mechanism of action and stage of clinical development. The drug repurposing tool was accessed at clue.io/repurposing-app.

Analysis of COVID-19 PBMC, Lung, and BAL DEG profiles via CLUE was performed as follows. DEGs from PBMC-CoV2 vs. PBMC-CTL, Lung-CoV2 vs. Lung-CTL, and BAL-CoV2 vs. PBMC-CoV2 were used as input for the CMaP and LINCS Unified Environment (CLUE) cloud-based connectivity map analysis platform (clue.io/connectopedia/). Top upregulated and downregulated DEGs from each signature as determined by magnitude of log 2 fold change were sequentially entered into CLUE until 150 of each were accepted for analysis to determine drugs, compounds, small molecules, and other perturbagens that mimic or oppose the uploaded COVID-19 gene expression signatures. Resultant drugs and compounds with negative connectivity scores in the [−75, −100] range were analyzed to include results with high confidence of antagonizing COVID-19 gene expression profiles.

Data Availability: the datasets analyzed in this study are available from the China National Center for Bioinformation's National Genomics Data Center, bigd.big.ac.cn/gsa/browse/CRA002390, and in the NCBI GEO repository www.ncbi.nlm.nih.gov/bioproject/PRJNA615032. Additional data generated or analyzed during this study are described by, for example, Daamen et al., “Comprehensive transcriptomic analysis of COVID-19 blood, lung, and airway”, Scientific Reports, Vol. 11, No. 7052 (2021), which is incorporated by reference herein in its entirety.

### REFERENCES

1. Greenberg, S. B. Update on Human Rhinovirus and Coronavirus Infections. Semin. Respir. Crit. Care Med. 37, 555-571 (2016), is incorporated by reference herein in its entirety.
2. Cui, J., Li, F. & Shi, Z. L. Origin and evolution of pathogenic coronaviruses. Nat. Rev. Microbiol. 17, 181-192 (2019), is incorporated by reference herein in its entirety.
3. Fung, T. S. & Liu, D. X. Human Coronavirus: Host-Pathogen Interaction. 529-560 (2019), is incorporated by reference herein in its entirety.
4. Zhang, B. et al. Clinical characteristics of 82 death cases with COVID-19. medRxiv 2020.02.26.20028191 (2020). doi:10.1101/2020.02.26.20028191, is incorporated by reference herein in its entirety.
5. Chen, G. et al. Clinical and immunologic features in severe and moderate Coronavirus Disease 2019. J. Clin. Invest. (2020). doi:10.1172/JCI137244, is incorporated by reference herein in its entirety.
6. Lazear, H. M., Schoggins, J. W. & Diamond, M. S. Shared and Distinct Functions of Type I and Type III Interferons. Immunity 50, 907-923 (2019), is incorporated by reference herein in its entirety.
7. Kelley, N., Jeltema, D., Duan, Y. & He, Y. The NLRP3 inflammasome: An overview of mechanisms of activation and regulation. Int. J. Mol. Sci. 20, 1-24 (2019).
8. Newton, A. H., Cardani, A. & Braciale, T. J. The host immune response in respiratory virus infection: balancing virus clearance and immunopathology. Semin. Immunopathol. 38, 471-482 (2016), is incorporated by reference herein in its entirety.
9. McGonagle, D., Sharif, K., O'Regan, A. & Bridgewood, C. The Role of Cytokines including Interleukin-6 in COVID-19 induced Pneumonia and Macrophage Activation Syndrome-Like Disease. Autoimmun. Rev. 102537 (2020). doi:10.1016/j.autrev.2020.102537, is incorporated by reference herein in its entirety.
10. Crayne, C. B., Albeituni, S., Nichols, K. E. & Cron, R. Q. The immunology of macrophage activation syndrome. Front. Immunol. 10, 1-11 (2019), is incorporated by reference herein in its entirety.
11. Xiong, Y. et al. Transcriptomic characteristics of bronchoalveolar lavage fluid and peripheral blood mononuclear cells in COVID-19 patients. Emerg. Microbes Infect. 9, 761-770 (2020), is incorporated by reference herein in its entirety.
12. Blanco-Melo, D. et al. Imbalanced host response to SARS-CoV-2 drives development of COVID-19. Cell (2020). doi:10.1016/j.cell.2020.04.026, is incorporated by reference herein in its entirety.
13. Wei, L. et al. Viral Invasion and Type I Interferon Response Characterize the Immunophenotypes During Covid-19 Infection. SSRN Electron. J. (2020). doi:10.2139/ssrn.3564998, is incorporated by reference herein in its entirety.
14. Catalina, M. D., Bachali, P., Geraci, N. S., Grammer, A. C. & Lipsky, P. E. Gene expression analysis delineates the potential roles of multiple interferons in systemic lupus erythematosus. Commun. Biol. 2, 140 (2019), is incorporated by reference herein in its entirety.
15. Catalina, M. D. et al. Patient ancestry significantly contributes to molecular heterogeneity of systemic lupus erythematosus. JCI Insight 5, (2020), is incorporated by reference herein in its entirety.
16. Wen, W. et al. Immune cell profiling of COVID-19 patients in the recovery stage by single-cell sequencing. Cell Discov. 6 (1) (2020), is incorporated by reference herein in its entirety.
17. Kegerreis, B. J. et al. Genomic Identification of Low-Density Granulocytes and Analysis of Their Role in the Pathogenesis of Systemic Lupus Erythematosus. J. Immunol. 202, 3309-3317 (2019), is incorporated by reference herein in its entirety.
18. Schulte-Schrepping, J. et al. Severe COVID-19 Is Marked by a Dysregulated Myeloid Cell Compartment. Cell 182, 1419-1440.e23 (2020), is incorporated by reference herein in its entirety.
19. Aschenbrenner, A. C. et al. Disease severity-specific neutrophil signatures in blood transcriptomes stratify COVID-19 patients. medRxiv 2020.07.07.20148395 (2020). doi:10.1101/2020.07.07.20148395, is incorporated by reference herein in its entirety.
20. Trouillet-Assant, S. et al. Type I IFN immunoprofiling in COVID-19 patients. J. Allergy Clin. Immunol. (2020). doi:10.1016/j.jaci.2020.04.029, is incorporated by reference herein in its entirety.
21. Fischer, H., Tschachler, E. & Eckhart, L. Pangolins Lack IFIH1/MDA5, a Cytoplasmic RNA Sensor That Initiates Innate Immune Defense Upon Coronavirus Infection. Front. Immunol. 11, 939 (2020), is incorporated by reference herein in its entirety.
22. Bojkova, D. et al. Proteomics of SARS-CoV-2-infected host cells reveals therapy targets. Nature (2020). doi:10.1038/s41586-020-2332-7, is incorporated by reference herein in its entirety.
23. Vazquez, C. & Homer, S. M. MAVS Coordination of Antiviral Innate Immunity. J. Virol. 89, 6974 LP-6977 (2015), is incorporated by reference herein in its entirety.
24. Lindell, D. M., Lane, T. E. & Lukacs, N. W. CXCL10/CXCR3-mediated responses promote immunity to respiratory syncytial virus infection by augmenting dendritic cell and CD8 (+) T cell efficacy. Eur. J. Immunol. 38, 2168-2179 (2008), is incorporated by reference herein in its entirety.
25. Teijaro, J. R. The role of cytokine responses during influenza virus pathogenesis and potential therapeutic options. Curr. Top. Microbiol. Immunol. 386, 3-22 (2015), is incorporated by reference herein in its entirety.
26. Sungnak, W. et al. SARS-CoV-2 entry factors are highly expressed in nasal epithelial cells together with innate immune genes. Nat. Med. 1-7 (2020). doi:10.1038/s41591-020-0868-6, is incorporated by reference herein in its entirety.
27. Liao, M. et al. The landscape of lung bronchoalveolar immune cells in COVID-19 revealed by single-cell RNA sequencing. medRxiv 2020.02.23.20026690 (2020). doi:10.1101/2020.02.23.20026690, is incorporated by reference herein in its entirety.
28. Suzuki, T. et al. Pulmonary macrophage transplantation therapy. Nature 514, 450-454 (2014), is incorporated by reference herein in its entirety.
29. Joshi, P. C. et al. GM-CSF receptor expression and signaling is decreased in lungs of ethanol-fed rats. Am. J. Physiol. Lung Cell. Mol. Physiol. 291, L1150-8 (2006), is incorporated by reference herein in its entirety.
30. Reyfman, P. A. et al. Single-Cell Transcriptomic Analysis of Human Lung Provides Insights into the Pathobiology of Pulmonary Fibrosis. Am. J. Respir. Crit. Care Med. 199, 1517-1536 (2019), is incorporated by reference herein in its entirety.
31. Hubbard, E. L. et al. Analysis of Gene Expression from Systemic Lupus Erythematosus Synovium Reveals Myeloid Cell-Driven Pathogenesis of Lupus Arthritis. Sci. Rep. 10, 1-17 (2020), is incorporated by reference herein in its entirety.
32. Kuo, D. et al. HBEGF+ macrophages in rheumatoid arthritis induce fibroblast invasiveness. Sci. Transl. Med. 11, eaau8587 (2019), is incorporated by reference herein in its entirety.
33. Martinez, F. O., Gordon, S., Locati, M. & Mantovani, A. Transcriptional profiling of the human monocyte-to-macrophage differentiation and polarization: new molecules and patterns of gene expression. J. Immunol. 177, 7303-7311 (2006), is incorporated by reference herein in its entirety.
34. Tay, M. Z., Poh, C. M., Rénia, L., MacAry, P. A. & Ng, L. F. P. The trinity of COVID-19: immunity, inflammation and intervention. Nat. Rev. Immunol. 1-12 (2020). doi:10.1038/s41577-020-0311-8, is incorporated by reference herein in its entirety.
35. Corley, M. J., Sugai, C., Schotsaert, M., Schwartz, R. E. & Ndhlovu, L. C. Comparative &ltem&gt;in vitro&lt;/em&gt; transcriptomic analyses of COVID-19 candidate therapy hydroxychloroquine suggest limited immunomodulatory evidence of SARS-CoV-2 host response genes. bioRxiv 2020.04.13.039263 (2020). doi:10.1101/2020.04.13.039263, is incorporated by reference herein in its entirety.
36. Viola, A., Munari, F., Sanchez-Rodriguez, R., Scolaro, T. & Castegna, A. The metabolic signature of macrophage responses. Front. Immunol. 10, 1-16 (2019), is incorporated by reference herein in its entirety.
37. Huang, C. et al. Clinical features of patients infected with 2019 novel coronavirus in Wuhan, China. Lancet 395, 497-506 (2020), is incorporated by reference herein in its entirety.
38. Barnes, B. J. et al. Targeting potential drivers of COVID-19: Neutrophil extracellular traps. J. Exp. Med. 217, 1-7 (2020), is incorporated by reference herein in its entirety.
39. Thierry, A. R. & Roch, B. NETs by-products and extracellular DNA may play a key role in COVID-19 pathogenesis: incidence on patient monitoring and therapy. 1-21 (2020). doi:10.20944/preprints202004.0238.v1, is incorporated by reference herein in its entirety.
40. Carmona-Rivera, C. & Kaplan, M. J. Low-density granulocytes: a distinct class of neutrophils in systemic autoimmunity. Semin. Immunopathol. 35, 455-463 (2013), is incorporated by reference herein in its entirety.
41. Tipping, P. G., Campbell, D. A., Boyce, N. W. & Holdsworth, S. R. Alveolar macrophage procoagulant activity is increased in acute hyperoxic lung injury. Am. J. Pathol. 131, 206-212 (1988), is incorporated by reference herein in its entirety.
42. Wichmann, D. et al. Autopsy Findings and Venous Thromboembolism in Patients With COVID-19: A Prospective Cohort Study. Ann. Intern. Med. (2020). doi:10.7326/M20-2003, is incorporated by reference herein in its entirety.
43. Allard, B., Panariti, A. & Martin, J. G. Alveolar Macrophages in the Resolution of Inflammation, Tissue Repair, and Tolerance to Infection. Front. Immunol. 9, 1777 (2018), is incorporated by reference herein in its entirety.
44. Chen, N. et al. Epidemiological and clinical characteristics of 99 cases of 2019 novel coronavirus pneumonia in Wuhan, China: a descriptive study. Lancet 395, 507-513 (2020), is incorporated by reference herein in its entirety.
45. Qin, C. et al. Dysregulation of immune response in patients with COVID-19 in Wuhan, China. Clin. Infect. Dis. 2019, 4-10 (2020), is incorporated by reference herein in its entirety.
46. Xu, Z. et al. Pathological findings of COVID-19 associated with acute respiratory distress syndrome. Lancet Respir. Med. 8, 420-422 (2020), is incorporated by reference herein in its entirety.
47. He, Z. et al. Effects of severe acute respiratory syndrome (SARS) coronavirus infection on peripheral blood lymphocytes and their subsets. Int. J. Infect. Dis. 9, 323-330 (2005), is incorporated by reference herein in its entirety.
48. Wu, F. et al. Neutralizing antibody responses to SARS-CoV-2 in a COVID-19 recovered patient cohort and their implications. medRxiv 2020.03.30.20047365 (2020). doi:10.1101/2020.03.30.20047365, is incorporated by reference herein in its entirety.
49. Iwasaki, A. & Yang, Y. The potential danger of suboptimal antibody responses in COVID-19. Nat. Rev. Immunol. 1-3 (2020). doi:10.1038/s41577-020-0321-6, is incorporated by reference herein in its entirety.
50. Liu, L. et al. Anti-spike IgG causes severe acute lung injury by skewing macrophage responses during acute SARS-CoV infection. JCI insight 4, 1-19 (2019), is incorporated by reference herein in its entirety.
51. Heron, M. et al. Bronchoalveolar lavage cell pattern from healthy human lung. Clin. Exp. Immunol. 167, 523-531 (2012), is incorporated by reference herein in its entirety.
52. Hamacher, J. et al. Tumor necrosis factor-α and angiostatin are mediators of endothelial cytotoxicity in bronchoalveolar lavages of patients with acute respiratory distress syndrome. Am. J. Respir. Crit. Care Med. 166, 651-656 (2002), is incorporated by reference herein in its entirety.
53. Pairo-Castineira, E. et al. Genetic mechanisms of critical illness in Covid-19. Nature (2020). doi:10.1038/s41586-020-03065-y, is incorporated by reference herein in its entirety.
54. Sun, X. et al. Cytokine storm intervention in the early stages of COVID-19 pneumonia. Cytokine Growth Factor Rev. (2020). doi:10.1016/j.cytogfr.2020.04.002, is incorporated by reference herein in its entirety.
55. Pedersen, S. F. & Ho, Y.-C. SARS-CoV-2: a storm is raging. J. Clin. Invest. 130, 2202-2205 (2020), is incorporated by reference herein in its entirety.
56. Voiriot, G. et al. Interleukin-6 displays lung anti-inflammatory properties and exerts protective hemodynamic effects in a double-hit murine acute lung injury. Respir. Res. 18, 64 (2017), is incorporated by reference herein in its entirety.
57. Mehta, P. et al. COVID-19: consider cytokine storm syndromes and immunosuppression. Lancet (London, England) 395, 1033-1034 (2020), is incorporated by reference herein in its entirety.
58. Gao, T. et al. Highly pathogenic coronavirus N protein aggravates lung injury by MASP-2-mediated complement over-activation. medRxiv 2020.03.29.20041962 (2020). doi:10.1101/2020.03.29.20041962, is incorporated by reference herein in its entirety.
59. Patterson, B. K. et al. Disruption of the CCL5/RANTES-CCR5 Pathway Restores Immune Homeostasis and Reduces Plasma Viral Load in Critical COVID-19. medRxiv 2020.05.02.20084673 (2020). doi:10.1101/2020.05.02.20084673, is incorporated by reference herein in its entirety.
60. Middeldorp, S. et al. Incidence of venous thromboembolism in hospitalized patients with COVID-19. J. Thromb. Haemost. (2020). doi:10.1111/jth.14888, is incorporated by reference herein in its entirety.
61. Klok, F. A. et al. Incidence of thrombotic complications in critically ill ICU patients with COVID-19. Thromb. Res. (2020). doi:10.1016/j.thromres.2020.04.013, is incorporated by reference herein in its entirety.
62. Geleris, J. et al. Observational Study of Hydroxychloroquine in Hospitalized Patients with Covid-19. N. Engl. J. Med. 0, null (2020), is incorporated by reference herein in its entirety.
63. Anders, S. & Huber, W. Differential expression analysis for sequence count data. Genome Biol. 11, R106 (2010), is incorporated by reference herein in its entirety.
64. Hanzelmann, S., Castelo, R. & Guinney, J. GSVA: gene set variation analysis for microarray and RNA-Seq data. BMC Bioinformatics 14, 7 (2013), is incorporated by reference herein in its entirety.
65. Kotliarov, Y. et al. Broad immune activation underlies shared set point signatures for vaccine responsiveness in healthy individuals and disease activity in patients with lupus. Nat. Med. (2020). doi:10.1038/s41591-020-0769-8, is incorporated by reference herein in its entirety.
66. Lukassen, S. et al. SARS-CoV-2 receptor ACE2 and TMPRSS2 are predominantly expressed in a transient secretory cell type in subsegmental bronchial branches. bioRxiv 2020.03.13.991455 (2020). doi:10.1101/2020.03.13.991455, is incorporated by reference herein in its entirety.
67. Labonte, A. C. et al. Identification of alterations in macrophage activation associated with disease activity in systemic lupus erythematosus. PLoS One 13, e0208132 (2018), is incorporated by reference herein in its entirety.
68. Trapnell, C. et al. The dynamics and regulators of cell fate decisions are revealed by pseudotemporal ordering of single cells. Nat. Biotechnol. 32, 381-386 (2014), is incorporated by reference herein in its entirety.
69. Qiu, X. et al. Single-cell mRNA quantification and differential analysis with Census. Nat. Methods 14, 309-315 (2017), is incorporated by reference herein in its entirety.
70. Qiu, X. et al. Reversed graph embedding resolves complex single-cell trajectories. Nat. Methods 14, 979-982 (2017), is incorporated by reference herein in its entirety.
71. Grammer, A. C. et al. Drug repositioning in SLE: crowd-sourcing, literature-mining and Big Data analysis. Lupus 25, 1150-1170 (2016), is incorporated by reference herein in its entirety.
72. Gu, Z., Eils, R. & Schlesner, M. Complex heatmaps reveal patterns and correlations in multidimensional genomic data. Bioinformatics 32, 2847-2849 (2016), is incorporated by reference herein in its entirety.
73. Morris, J. H. et al. clusterMaker: a multi-algorithm clustering plugin for Cytoscape. BMC Bioinformatics 12, 436 (2011), is incorporated by reference herein in its entirety.

FIG. 17 shows conserved and differential enrichment of immune cells and pathways in blood, lung, and airway of SARS-CoV2-infected patients. (sections a-d) Individual sample gene expression from the blood (section a), lung (section b), and airway (section c) was analyzed by GSVA for enrichment of immune cell and inflammatory pathways. The corresponding heatmap was generated using the R Bioconductor package complexHeatmap (v2.5.6) (Ref 72). Select enrichment scores are shown as violin plots in (section d) generated using GraphPad Prism v8.4.2 (www.graphpad.com). *p<0.05, **p<0.01.

FIGS. 18A-18C show elevated IFN expression in the lung tissue of COVID-19 patients. FIG. 18A: Normalized log 2 fold change RNA-seq expression values for IFN-associated genes from blood, lung, and airway of individual COVID-19 patients. The dotted line represents the expression of each gene in healthy individuals (for blood and lung) or PBMCs from COVID-19 patients (airway). FIG. 18B: Individual sample gene expression from the blood, lung, and airway was analyzed by GSVA for enrichment of IFN-related gene signatures. FIG. 18C: Normalized log 2 fold change RNA-seq expression values for anti-viral genes as in (section a). Generated using GraphPad Prism v8.4.2 (www.graphpad.com). #p<0.2, ##p<0.1, *p<0.05, **p<0.01, ***p<0.001, ****p<0.0001

FIGS. 19A-19D show that viral entry gene expression correlates with enhanced expression of inflammatory mediators in SARS-CoV2-infected lungs. FIGS. 19A-19B: Normalized log 2 fold change RNA-seq expression values for chemokines and chemokine receptors (section a) and IL-1 family members (FIG. 19B) from blood, lung, and airway of COVID-19 patients as in FIG. 18A. FIG. 19C: Individual sample gene expression from the blood, lung, and airway was analyzed by GSVA for enrichment of various lung tissue cell categories. FIG. 19D: Normalized log 2 fold change RNA-seq expression values for viral entry genes as in FIGS. 19A-19B. Generated using GraphPad Prism v8.4.2 (www.graphpad.com). #p<0.2, ##p<0.1, *p<0.05, **p<0.01, ***p<0.001, ****p<0.0001

FIGS. 20A-20F show that PPI analysis identifies different myeloid cell subsets and metabolic pathways in blood, lung, and airway of COVID-19 patients. DE upregulated genes from blood (FIG. 20A), lung (FIG. 20B), and airway (FIG. 20C) were used to create PPI metaclusters using Cytoscape (version 3.6.1) and the clusterMaker2 (version 1.2.1) plugin (Ref. 73). Size indicates the number of genes per cluster, color indicates the number of intra-cluster connections and edge weight indicates the number of inter-cluster connections. Enrichment for biological function and immune cell type was determined by BIG-C and I-Scope, respectively. Small clusters (˜14 genes) with similar function are grouped in dotted-line boxes. Clusters enriched in Mo/myeloid genes were combined by decreasing cluster stringency to create a new set of myeloid-derived metastructures from the blood (FIG. 20D), lung (FIG. 20E), and airway (FIG. 20F). Interaction scores showing the strength of interaction between clusters are indicated (0.4-0.6, medium interaction; 0.61-0.8, strong interaction; 0.81-0.99, very strong interaction).

FIGS. 21A-21F show that different co-expression-derived myeloid populations are found in blood, lung, and airway of COVID-19 patients. (FIG. 21A) GSVA enrichment of myeloid subpopulations increased in COVID-19 blood (A1), lung (A2), and airway (A3). (FIG. 21B) Venn Diagram of the gene overlap between myeloid subpopulations A1-A3. (FIG. 21C) Comparison of normalized log 2 fold change expression values of genes defining A1-A3. Expression values for each sample in each comparison were normalized by the mean of the log 2 fold change expression of FCGR1A, FCGR2A, and FCGR2C. Significant comparisons are displayed by Hedge's G effect size. (FIGS. 21D-21E) Characterization of A1-A3 by enrichment of myeloid populations (FIG. 21D) and PBMC, lung, and BAL myeloid metaclusters from FIGS. 20D-20F (FIG. 21E). Fisher's Exact Test was used to calculate overlap between transcriptomic signatures and significant overlaps (p<0.05) are shown as the negative logarithm of the p-value. (FIG. 21F) Trajectory analysis using expression of 621 genes (196 myeloid-specific genes used in a,b+425 additional myeloid genes shown in Tables 7A-7C) in the blood, lung, and airway compartments. Colors represent sample identity and size represents pseudotime distance along the trajectory. Generated using GraphPad Prism v8.4.2 (www.graphpad.com) and the R package Monocle v2.14.0 68-70.

FIG. 22 shows an analysis of biological activities of myeloid subpopulations. Linear regression between GSVA scores for each of the tissue-specific myeloid populations (A1-A3) and metabolism, NLRP3 Inflammasome, complement, apoptosis, and TNF signaling. Generated using GraphPad Prism v8.4.2 (www.graphpad.com).

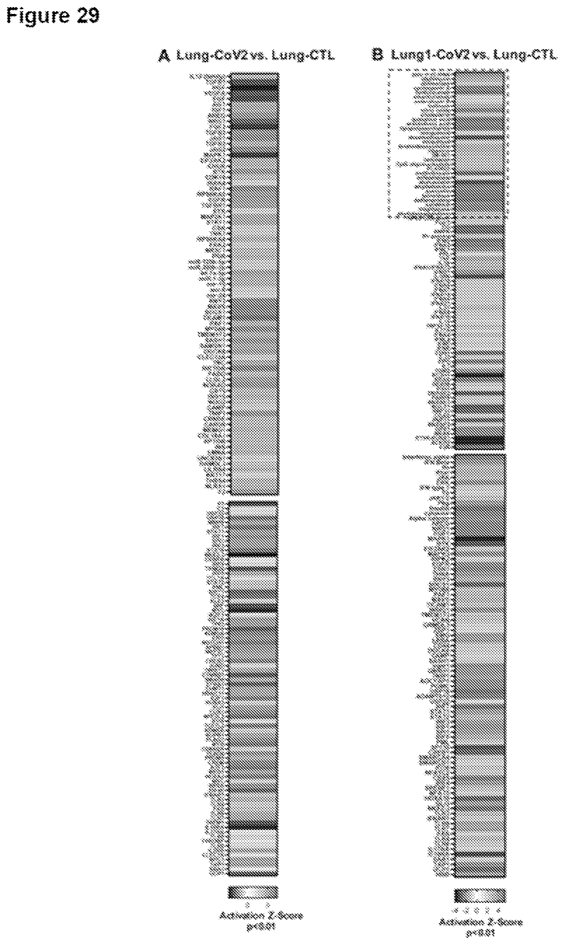

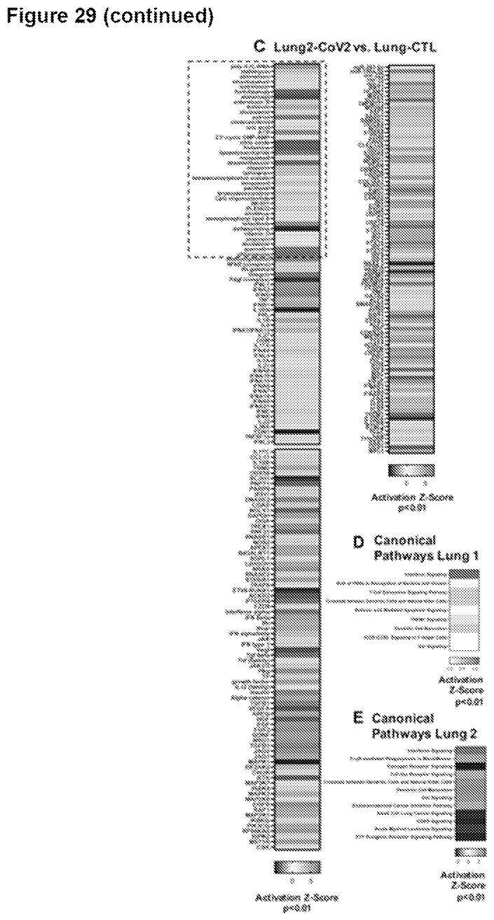

FIGS. 23A-23B show a pathway analysis of SARS-CoV-2 blood, lung, and airway. DEGs from each SARS-CoV-2 blood or tissue pairwise comparison were uploaded into IPA (Qiagen Inc., www.qiagenbioinformatics.com/products/ingenuity-pathway-analysis) and canonical signaling pathway (FIG. 23A) and upstream regulator (FIG. 23B) analyses were performed. Heatmaps represent significant results by Activation Z-Score |≥2| and overlap p-value<0.01. The boxes with the dotted outline separate drugs that were predicted as upstream regulators from pathway molecules and complexes. The remaining, significant upstream regulators were matched with drugs with known antagonistic targeting mechanisms. The top 150 UPRs in the lung are shown in (FIG. 23B) and the remaining are in FIGS. 29A-29E. Specific drugs for particular drug families (e.g., Anti-IL17) are found in Tables 8A-8B.

†: FDA-approved

‡: Drug in development/clinical trials

FIG. 24 shows a graphical model of COVID-19 pathogenesis. Proposed model of the inflammatory response to SARS-CoV2 infection in three compartments: the blood, lung, and airway generated using Microsoft PowerPoint version 19.0 and Adobe Illustrator version 25.0.

FIGS. 25A-25D show that metaclusters identify differentially expressed cell populations and functional gene clusters in SARS-CoV2 infected tissues and cell lines. Downregulated DE genes from peripheral blood (FIG. 25A), lung (FIG. 25B), and airway (FIG. 25C), and up-regulated DE genes from the NHBE primary lung epithelial cell line (FIG. 25D) were used to create metaclusters. Metaclusters were generated based on PPI networks, clustered using MCODE and visualized in Cytoscape as in FIGS. 20A-20F. Size indicates the number of genes per cluster, color indicates the number of intra-cluster connections and edge weight indicates the number of inter-cluster connections. Cluster enrichment for biological function and immune cell type was determined by BIG-C and I-Scope, respectively.

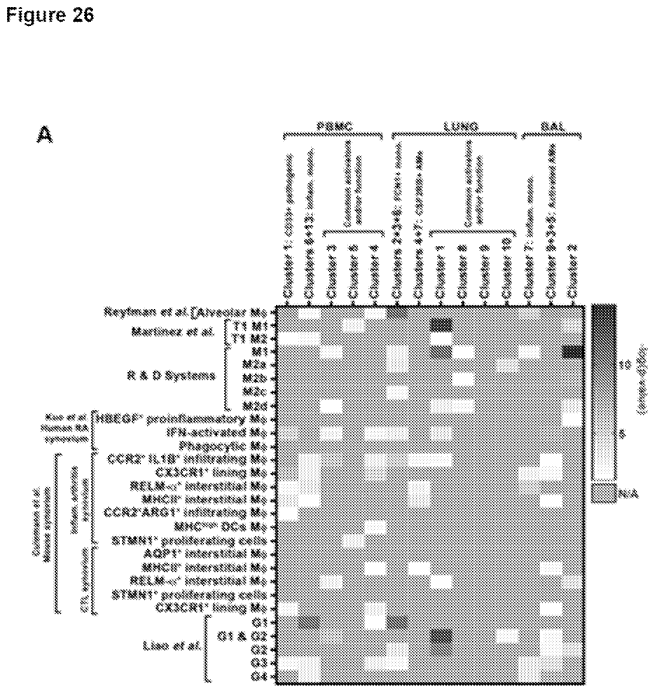

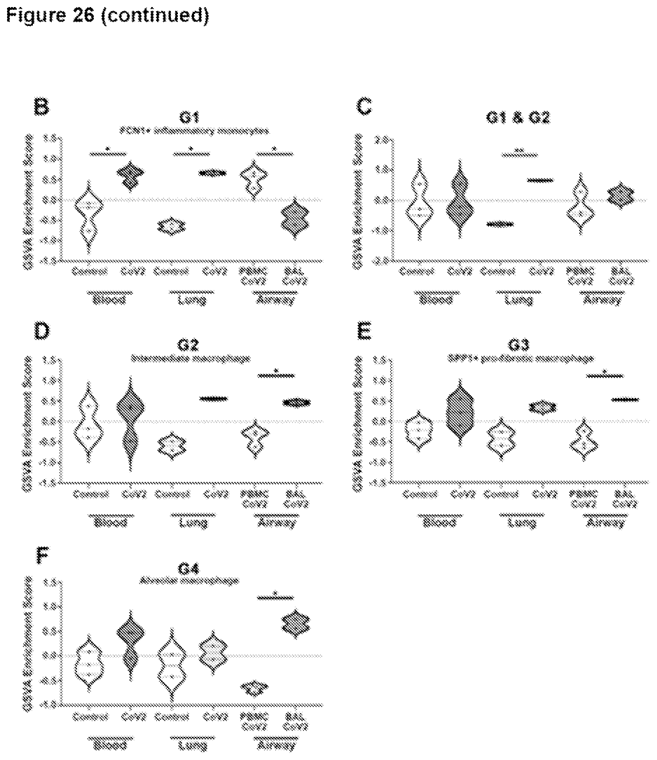

FIGS. 26A-26F show an evaluation of macrophage gene signatures in myeloid-derived clusters from COVID-affected blood, lung and BAL fluid. Macrophage signatures from the indicated sources were compared to myeloid clusters from FIGS. 19A-19D. Heatmap depicts signatures with significant overlap (−log (p-value)>1.33) with myeloid clusters from the blood, lung and airway compartments generated using GraphPad Prism v8.4.2 (www.graphpad.com). N/A, non-applicable/non-significant overlap detected. R & D Systems provided signatures for the M1, M2a, M2b, M2c and M2d populations.

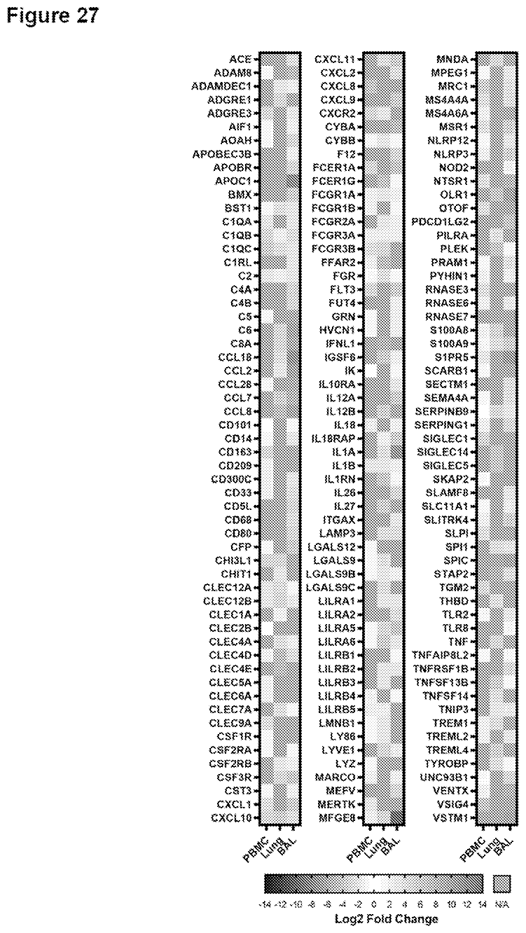

FIG. 27 shows heterogeneous expression of monocyte/myeloid cell genes in different CoV2 tissue compartments as compared to control. Evaluation of differential expression of 171 monocyte/myeloid genes in each compartment reveals shared and disparate expression among the tissues. PBMC represents PBMC-CoV2 to PBMC-CTL. Lung represents Lung-CoV2 to Lung-CTL. BAL represents BAL-CoV2 to PBMC-CoV2. Scale bar presents Log 2 Fold Change. N/A represents genes that were not significantly DE at FDR<0.2. Heatmaps generated using GraphPad Prism v8.4.2 (www.graphpad.com).

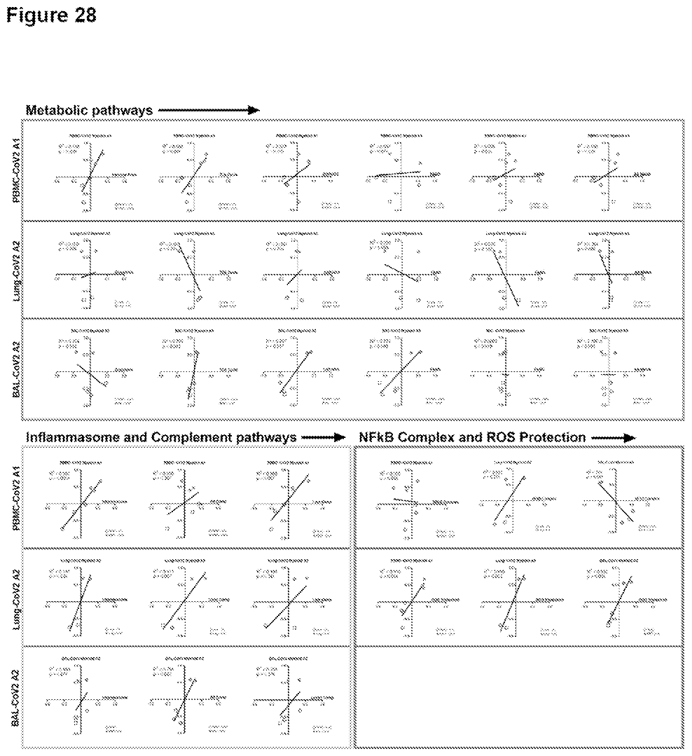

FIG. 28 shows an analysis of biological activities of myeloid subpopulations. Linear regression between GSVA scores for each of the tissue-specific myeloid populations and metabolic pathways, inlammasome, complement pathways, NFκB complex signaling and ROS protection. Generated using GraphPad Prism v8.4.2 (www.graphpad.com).

FIGS. 29A-29E show a pathway analysis of SARS-CoV-2 lung tissue. FIG. 29A: Remaining significant upstream regulators operative in SARS-CoV-2 lung tissue predicted by IPA upstream regulator analysis. Upstream regulator analysis was also conducted on DEGs from each individual COVID-19 lung compared to healthy controls due to observed heterogeneity FIG. 29B: significant results displayed for Lung1-CoV2 vs. Lung-CTL. FIG. 29C: significant results displayed for Lung2-CoV2 vs. Lung-CTL. Chemical reagents, chemical toxicants, and non-mammalian endogenous chemicals were culled from results. The boxes with the dotted outline separate small molecules/drugs/compounds that were predicted as upstream regulators from pathway molecules and complexes. FIGS. 29D-29E: IPA canonical signaling pathway analysis was conducted on individual COVID-19 lung samples. Pathways and upstream regulators were considered significant by |Activation Z-Score|≥2 and overlap p-value <0.01.

TABLE 5
Summary of datasets used

| | | | RNA- |
|---|---|---|---|
| | | | seq |
| Dataset | Accessibility | Metadata | Method |
| CRA002309 | bigd.big.ac.cn/gsa/ | 3 PBMC | Illumina |
| | browse/CRA002390 | SARS- | NovaSeq |
| | | CoV-2 | 5000 |
| | | 3 PBMC | |
| | | healthy | |
| | | donor | |
| | | 3 BALF | |
| | | SARS- | |
| | | CoV-2 | |
| GSE147505 | www.ncbi.nlm.nih.gov/ | 2 | Illumina |
| | bioproject/PRJNA615032 | postmortem | NextSeq |
| | | lung | 500 |
| | | 2 healthy | |
| | | lung | |

General COVID Patient/Sample Information

| | | | | Sample |
|---|---|---|---|---|
| | | | Age | Collection and |
| Dataset | Cohort | Gender | (years) | Isolation |
| CRA002309 | PBMC CoV2 | 3 Males | 25-74 | PBMC were |
| | | | | isolated using |
| | | | | Ficoll density |
| | | | | gradient method |
| | PBMC CTL | 3 Unknown | Unknown | |
| | | Gender | | |
| | BAL CoV2 | 3 Males | 25-74 | BAL fluid was |
| | | | | retrieved by |
| | | | | gentle syringe |
| | | | | suction using |
| | | | | sterile saline |
| GSE147505 | Lung CoV2 | 2 Males | >60 | Acquired post- |
| | | | | mortem as fixed |
| | | | | lung samples |
| | Lung CTL | 2 Males | >70 | Acquired from |
| | | | | biopsies of |
| | | | | uninfected lungs |
| | | | | obtained post- |
| | | | | surgery |

TABLE 6
(DEGs in Blood, Lung, and Airway)
PBMC-CoV2 to PBMC-CTL (4,245 DEGs in Blood)
A2M, AAGAB, AAK1, AAMP, AAR2, ABCA2, ABCA3, ABCA5, ABCA6, ABCA7,
ABCB1, ABCB7, ABCC2, ABCD1, ABHD15, ABHD17A, ABHD6, ABI2, ABITRAM,
ABLIM1, ABRACL, ABT1, ACAA2, ACACB, ACAD10, ACADSB, ACADVL, ACAP1,
ACCS, ACD, ACER3, ACHE, ACLY, ACO2, ACOT2, ACOT8, ACOT9, ACOX3, ACP2,
ACP5, ACSL3, ACSS2, ACTA1, ACTB, ACTG1, ACTR1A, ACTR1B, ACTR2, ACTR5,
ACVRIB, ACVRIC, ACVR2B, ACVRL1, ACYP1, ADA2, ADAM15, ADAM19,
ADAM22, ADAM23, ADAM8, ADAM9, ADAMTS10, ADAMTS17, ADAMTS2,
ADAMTS4, ADAMTSL5, ADAP2, ADARB1, ADGRB2, ADGRE3, ADGRE5, ADGRG1,
ADGRG5, ADGRL1, ADGRL2, ADH5, ADIPOR1, ADORA2A, ADORA3, ADPGK,
ADPRH, ADRM1, AEBP2, AFAP1, AFMID, AGAP1, AGAP2, AGAP9, AGBL3, AGFG1,
AGFG2, AGK, AGL, AGPAT2, AGPAT3, AGRN, AGTRAP, AHCY, AHCYL1, AHDC1,
AIF1, AIFM1, AIMP2, AJM1, AK2, AK4, AK5, AKAP12, AKAP17A, AKAP5, AKAP9,
AKIP1, AKNA, AKR1A1, AKR1C3, AKT3, ALAD, ALAS1, ALCAM, ALDH1A1,
ALDH1L2, ALDH2, ALDH3A2, ALDH3B1, ALDH4A1, ALDH9A1, ALDOA, ALG1,
ALG14, ALG8, ALKBH4, ALOX15, ALOX15B, ALOX5, ALPL, ALS2CL, AMD1,
AMDHD2, AMIGO1, AMIGO2, AMMECR1, AMN, AMOT, AMPD2, AMPH, ANAPC15,
ANG, ANGPTL6, ANK1, ANK3, ANKLE1, ANKMY1, ANKRA2, ANKRD11, ANKRD12,
ANKRD22, ANKRD34A, ANKRD34B, ANKRD36, ANKRD36B, ANKRD36C, ANKRD37,
ANKRD39, ANKRD44, ANKRD46, ANKRD49, ANKRD50, ANKRD52, ANKRD54,
ANKRD55, ANKS3, ANKS6, ANKZF1, ANLN, ANO10, ANO5, ANO9, ANXA2, ANXA4,
ANXA5, ANXA7, AOAH, AOC2, AOC3, AP1B1, AP1S1, AP2M1, AP2S1, AP3M1,
AP3M2, AP3S1, AP4S1, APBA3, APBB1, APC2, APEX2, APH1A, API5, APLP2, APMAP,
APOE, APOL4, APOLD1, APP, ARCN1, AREG, ARF1, ARF4, ARGLU1, ARHGAP12,
ARHGAP15, ARHGAP18, ARHGAP23, ARHGAP24, ARHGAP25, ARHGAP29,
ARHGAP31, ARHGAP33, ARHGAP4, ARHGAP5, ARHGAP9, ARHGEF1, ARHGEF11,
ARHGEF18, ARHGEF19, ARHGEF3, ARHGEF35, ARHGEF5, ARHGEF9, ARID3B,
ARID4A, ARID4B, ARID5B, ARL10, ARL11, ARL4A, ARL4C, ARL6, ARL6IP1, ARL8B,
ARMC2, ARMH3, ARMT1, ARNT, ARNTL2, ARPC1B, ARPC3, ARPC5, ARPIN,
ARRDC3, ARRDC4, ARSA, ARSB, ARV1, ARVCF, ASAH1, ASAH2B, ASB13, ASCL2,
ASGR1, ASGR2, ASH2L, ASL, ASPH, ASPM, ASPRV1, ASTE1, ATAD3B, ATF4, ATF7,
ATG12, ATG16L1, ATG2A, ATG3, ATG4A, ATG7, ATG9B, ATM, ATN1, ATP10D,
ATP11A, ATP1B3, ATP23, ATP2A2, ATP2A3, ATP2B1, ATP2B4, ATP5F1A, ATP5F1B,
ATP5F1C, ATP5F1E, ATP5MC1, ATP5MC2, ATP5MC3, ATP5MF, ATP5MG, ATP5MPL,
ATP5PB, ATP5PD, ATP5PF, ATP6AP1, ATP6AP2, ATP6V0A1, ATP6V0A2, ATP6V0D1,
ATP6V0E1, ATP6V0E2, ATP6V1A, ATP6V1B2, ATP6V1C1, ATP6V1D, ATP6V1E1,
ATP6V1F, ATP8B1, ATP8B2, ATPSCKMT, ATRX, ATXN10, ATXN7L2, ATXN7L3B,
AURKA, AURKB, AUTS2, AVIL, AVPR2, AXIN1, AXIN2, AZI2, AZIN1, AZIN2, B2M,
B3GAT1, B3GLCT, B4GALNT4, B4GAT1, B9D2, BACE1, BACH1, BACH2, BAG5,
BAHCC1, BAHD1, BAK1, BAMBI, BASP1, BATF2, BAX, BBX, BCAS2, BCAT1,
BCKDHA, BCKDHB, BCKDK, BCL11B, BCL2A1, BCL2L13, BCL7C, BCL9L, BCR,
BDP1, BEGAIN, BEX2, BEX4, BEX5, BFAR, BFSP1, BHLHA15, BICDL1, BICRA, BIN1,
BIN3, BIRC5, BLM, BLNK, BLOC1S4, BLVRA, BLVRB, BMERB1, BMF, BMI1,
BMP2K, BMPR1A, BNC2, BNIP2, BNIP3, BOD1L1, BPI, BPTF, BRCA1, BRCA2, BRD1,
BRD9, BRF2, BRI3, BRICD5, BRIX1, BRK1, BRPF3, BSG, BST1, BST2, BTBD2, BTBD3,
BTBD9, BTF3L4, BTG1, BTG2, BTK, BTN3A1, BTN3A2, BTNL8, BUB1, BUB1B, BUB3,
BZW2, C11orf24, C11orf65, C12orf29, C12orf4, C12orf57, C12orf75, C13orf46, C14orf119,
C14orf132, C14orf28, C15orf41, C16orf54, C16orf70, C16orf91, C17orf107, C17orf58,
C17orf67, C19orf12, C19orf25, C19orfi8, C19orf54, C19orf71, C19orf84, C1GALT1C1,
C1orf115, C1orf162, C1orf21, C1orf216, C1orf35, C1orf43, C1orf50, C1QA, C1QB, C1QC,
C2, C20orf194, C20orf204, C20orf27, C20orf96, C21orf91, C22orf19, C2CD5, C2orf49,
C3AR1, C4orf50, C5, C5orf15, C5orf22, C5orf24, C5orf30, C6orf136, C7orf26, C8orf58,
C9orf139, C9orf64, C9orf78, CA11, CA2, CA5B, CA8, CABIN1, CABP4, CACNA1H,
CACNA1I, CACNA2D2, CACNA2D4, CACNB1, CACNB2, CACNG8, CACTIN,
CALCOCO2, CALCRL, CALM2, CALU, CAMK1, CAMK1D, CAMK2G, CAMK2N1,
CAMK4, CAMP, CAP1, CAPG, CAPN1, CAPN10, CAPN15, CAPN2, CAPN3, CAPNS1,
CAPRIN1, CAPRIN2, CAPZA1, CAPZA2, CARD11, CARD16, CARD6, CARMIL2,
CARNMT1, CARNS1, CASD1, CASP1, CASP7, CASP8, CASP8AP2, CASS4, CAST,
CASTOR3, CAT, CATSPER1, CATSPER2, CATSPERB, CATSPERG, CBFA2T2, CBLB,
CBR1, CBR4, CBWD1, CBWD2, CBWD3, CBWD5, CBWD6, CBX4, CBX7, CC2D1B,
CCDC102A, CCDC12, CCDC120, CCDC136, CCDC138, CCDC141, CCDC146, CCDC149,
CCDC157, CCDC159, CCDC17, CCDC18, CCDC191, CCDC200, CCDC28A, CCDC43,
CCDC47, CCDC57, CCDC59, CCDC6, CCDC65, CCDC66, CCDC69, CCDC7, CCDC78,
CCDC80, CCDC84, CCDC88A, CCDC88B, CCDC88C, CCDC91, CCDC92, CCL28,
CCL3L1, CCL4, CCL4L2, CCL5, CCM2, CCN3, CCNA2, CCNB1, CCNB2, CCND2,
CCNJ, CCNL2, CCNT1, CCNYL1, CCR1, CCR2, CCR3, CCR7, CCR9, CCRL2, CCSAP,
CCSER2, CCT5, CCT6A, CCT8, CCZ1, CCZ1B, CD101, CD109, CD14, CD151, CD160,
CD163, CD163L1, CD164, CD180, CD1A, CD1D, CD2, CD200R1, CD209, CD226, CD244,
CD247, CD28, CD2BP2, CD300C, CD300LB, CD302, CD320, CD33, CD36, CD38, CD3D,
CD3E, CD3G, CD40LG, CD47, CD5, CD6, CD63, CD69, CD7, CD81, CD86, CD93, CD96,
CD99L2, CDAN1, CDC14A, CDC20, CDC25A, CDC25B, CDC37L1, CDC40, CDC42BPA,
CDC42BPB, CDC42BPG, CDC42EP1, CDC42EP3, CDC42SE1, CDC42SE2, CDC45,
CDC6, CDCA3, CDCA5, CDCA8, CDH24, CDHR1, CDHR3, CDIP1, CDK1, CDK11B,
CDK13, CDK17, CDK20, CDK2AP2, CDK5, CDK5RAP2, CDK9, CDKN1B, CDKN1C,
CDKN2AIP, CDKN2B, CDKN2C, CDT1, CEACAM1, CEACAM3, CEBPA, CEBPD,
CENPF, CENPN, CENPT, CENPU, CENPV, CEP104, CEP120, CEP126, CEP131, CEP135,
CEP152, CEP164, CEP170, CEP250, CEP290, CEP55, CEP68, CEP70, CEP78, CEP85L,
CERCAM, CERK, CERS4, CERS6, CES1, CES1P1, CFAP20, CFAP298, CFAP36,
CFAP410, CFAP44, CFAP45, CFH, CFP, CH25H, CHCHD1, CHCHD4, CHCHD6, CHD2,
CHD3, CHD6, CHD7, CHERP, CHI3L1, CHIC1, CHID1, CHKA, CHML, CHMP2B,
CHMP4A, CHP1, CHRFAM7A, CHRNA2, CHRNA7, CHRNE, CHST10, CHST12,
CHST13, CHST2, CHST7, CHST8, CHTF18, CHTF8, CIAO2A, CIC, CINP, CIP2A, CIRBP,
CISD1, CISH, CIT, CKAP5, CKB, CKS2, CLASRP, CLC, CLCF1, CLCN3, CLCN4,
CLCN5, CLCN6, CLCN7, CLDN15, CLDND2, CLEC12A, CLEC12B, CLEC18A,
CLEC18B, CLEC1B, CLEC2B, CLEC2D, CLEC4C, CLEC4D, CLEC40, CLEC5A,
CLEC6A, CLEC9A, CLHC1, CLIC1, CLIC3, CLIC4, CLIC5, CLK1, CLN5, CLN6, CLN8,
CLNS1A, CLPB, CLPTM1, CLPTM1L, CLSPN, CLSTN1, CLSTN3, CLTA, CLTC,
CLTCL1, CMAS, CMC1, CMC2, CMKLR1, CMPK1, CMPK2, CMTM2, CMTM4,
CMTM7, CNDP2, CNIH3, CNIH4, CNNM3, CNOT6L, CNP, CNPY4, CNTLN, CNTNAP1,
CNTNAP2, COA3, COA4, COA5, COA7, COASY, COG8, COL13A1, COL18A1, COL6A1,
COL6A2, COL8A2, COLGALT1, COLGALT2, COLQ, COMMD1, COMMD10,
COMMD9, COMT, COPA, COPB2, COPG1, COPG2, COPS2, COPS3, COPZ1, COQ10A,
COQ2, COQ5, CORO1C, CORO2B, CORO6, CORO7, COTL1, COX10, COX15, COX5A,
COX5B, COX6A1, COX6B1, COX7A2L, COX7B, COX8A, CPA3, CPEB3, CPEB4,
CPED1, CPM, CPNE2, CPNE8, CPSF1, CPSF2, CPSF3, CPVL, CRACR2A, CRAT,
CREB1, CREBRF, CREBZF, CREG1, CRELD1, CREM, CRIM1, CRIP1, CRIP2,
CRISPLD2, CRLF3, CRLS1, CROCC, CROCC2, CROT, CRTAP, CRTC2, CRY1,
CRYBG1, CRYBG2, CRYBG3, CRYM, CS, CSF1, CSF1R, CSF2RA, CSGALNACT2,
CSNK1A1, CSNK1E, CSNK1G2, CSNK2A1, CSNK2A3, CSPP1, CSRNP1, CST3, CST7,
CSTA, CSTB, CTAGE15, CTAGE4, CTAGE6, CTAGE8, CTBP1-AS, CTC1, CTDSPL2,
CTIF, CTNNA1, CTNNB1, CTNND1, CTNS, CTSA, CTSB, CTSC, CTSD, CTSF, CTSH,
CTSL, CTSO, CTSS, CTSW, CTSZ, CTTNBP2NL, CUBN, CUEDC2, CUL1, CUX1,
CWC15, CWF19L2, CXCL1, CXCL10, CXCL5, CXCL8, CXCR1, CXCR2, CXorf21,
CXorf38, CYB561D1, CYB561D2, CYB5R1, CYB5R3, CYBB, CYBRD1, CYC1, CYCS,
CYFIP1, CYFIP2, CYHR1, CYLD, CYP19A1, CYP1B1, CYP2D6, CYP2D7, CYP4F12,
CYSLTR2, CYTH1, CYTH2, DAAM2, DAB2, DAD1, DAGLA, DALRD3, DAP, DAPK2,
DARS2, DBF4, DBH, DBI, DBN1, DBP, DCAF15, DCAF16, DCLRE1A, DCPS, DCTN2,
DCTN4, DCTN5, DCTN6, DDB1, DDHD2, DDI2, DDIT4, DDOST, DDX17, DDX21,
DDX41, DDX49, DDX5, DDX6, DECR1, DEDD2, DEF6, DEFA1, DEFA1B, DEFA3,
DENND10, DENND11, DENND1A, DENND1C, DENND2D, DENND5B, DEPP1, DERA,
DERL3, DESI1, DGKE, DGKG, DGKK, DGKQ, DGKZ, DGUOK, DHCR24, DHFR,
DHRS11, DHRS13, DHRS3, DHRS4, DHRS4L2, DHX29, DHX32, DHX33, DHX57,
DHX9, DIAPH2, DIDO1, DIMT1, DIP2C, DLC1, DLD, DLEC1, DLEU7, DLG3, DLG4,
DLG5, DLGAP5, DMAC2, DMAP1, DMPK, DMWD, DMXL1, DNAAF2, DNAH10,
DNAH8, DNAJA1, DNAJA2, DNAJB1, DNAJB12, DNAJB13, DNAJB14, DNAJB9,
DNAJC13, DNAJC15, DNAJC24, DNAJC5, DNAJC6, DNAL1, DNASE1, DNASE1L1,
DNASE2, DNM1L, DNMT3A, DOCK1, DOCK2, DOCK7, DOCK8, DOCK9, DOK1,
DOK4, DOK6, DOLPP1, DOP1B, DPAGT1, DPEP2, DPEP3, DPP3, DPP9-AS1, DPYD,
DPYSL2, DRAM1, DRAM2, DRAXIN, DSE, DSTN, DTHD1, DTL, DTWD1, DTWD2,
DTX3, DTX3L, DUOX1, DUS3L, DUSP16, DUSP2, DUSP3, DUSP7, DUSP8, DVL1,
DVL2, DXO, DYM, DYNLL1, DYNLL2, DYNLT3, DYRK1A, DYRK1B, DYRK2,
DYRK3, DYSF, DZIP3, E2F1, E2F2, E2F3, E2F4, E2F6, E4F1, EAF1, EAF2, EBF4,
EBLN2, ECH1, ECHDC1, ECHDC2, ECHDC3, ECPAS, EDAR, EDEM2, EDEM3,
EDNRB, EED, EEF1AKNMT, EFHC2, EFHD2, EFNA5, EFTUD2, EGFL7, EGLN1, EGR3,
EHD4, EID3, EIF1, EIF1AD, EIF1AX, EIF2AK1, EIF2AK4, EIF2B1, EIF2B2, EIF2S1,
EIF2S3, EIF2S3B, EIF3I, EIF3M, EIF4E2, EIF4EBP1, EIF4G1, EIF4H, EIF5A2, EIF5AL1,
EIF6, ELAVL1, ELK4, ELL2, ELMO2, ELMO3, ELOB, ELOC, ELOVL1, ELP2, EMC7,
EMC8, EMID1, EMILIN2, EML3, EML4, EMP3, ENKUR, ENO1, ENO2, ENO3, ENOX2,
ENPP3, ENPP5, ENY2, EOMES, EPB41L3, EPB41L4A, EPC1, EPDR1, EPHA4, EPHB1,
EPHB2, EPHB4, EPHX2, EPN2, EPPK1, EPS8, EPS8L2, EPSTI1, ERAL1, ERBB2,
ERCC6L2, ERCC8, EREG, ERFL, ERG28, ERH, ERI2, ERLIN1, ERLIN2, ERMAP, ERN1,
ERO1A, ERO1B, ERP27, ERP44, ESCO1, ESD, ESF1, ESPL1, ESR1, ESR2, ESRP2,
ESYT2, ETAA1, ETF1, ETFA, ETFDH, ETHE1, ETS1, ETS2, EVI2A, EVI2B, EVI5, EVL,
EWSR1, EXD3, EXOC4, EXOC6, EXOC8, EXOSC1, EXOSC4, EXOSC6, EXPH5, EXT1,
EXT2, EZH2, F2RL2, F5, FABP5, FADS1, FAH, FAIM, FAM102A, FAM107B, FAM118B,
FAM122B, FAM131B, FAM151B, FAM153B, FAM156A, FAM156B, FAM160B2,
FAM169A, FAM172A, FAM174A, FAM177A1, FAM193B, FAM199X, FAM200B,
FAM20A, FAM20C, FAM214A, FAM229A, FAM241A, FAM32A, FAM43A, FAM72A,
FAM72B, FAM72C, FAM72D, FAM76A, FAM76B, FAM83A, FAM89A, FAM98C,
FANCA, FANCI, FAT2, FAT4, FAXDC2, FBF1, FBL, FBLN7, FBN2, FBP1, FBRSL1,
FBXL13, FBXL14, FBXL15, FBXL16, FBXL17, FBXL5, FBXL8, FBX03, FBXO31,
FBXO32, FBXO33, FBXO45, FBXO5, FBXO6, FCER1A, FCGR1A, FCGR1B, FCGR3A,
FCGR3B, FCGRT, FCHO1, FCHSD1, FCMR, FCN1, FCN2, FCRL3, FCRL5, FCRL6,
FEN1, FERMT3, FES, FEZ1, FEZ2, FFAR2, FGD1, FGD2, FGD3, FGD4, FGD6, FGFBP2,
FGFR1OP, FGFRL1, FGR, FH, FHDC1, FHL1, FIBP, FIG4, FIS1, FKBP15, FKBP1A,
FKBP1B, FKBP5, FKBP9, FLT1, FLT3, FLVCR2, FLYWCH1, FMN1, FMNL3, FMO5,
FN1, FNBP1L, FNDC9, FNIP2, FOSB, FOXD2-AS1, FOXK1, FOXM1, FOXN2, FOXO1,
FOXP1, FOXP3, FOXP4, FPGT, FPR3, FRMD3, FRMD4B, FRMD8, FRMPD3, FRRS1,
FSCN1, FSTL3, FTL, FUCA2, FURIN, FUT11, FXYD6, FYB1, FYN, FZD1, FZD2, FZD3,
FZD5, FZD7, GOS2, G2E3, G6PC3, G6PD, GAA, GABRR2, GADD45B, GAL3ST4,
GALK1, GALK2, GALNT11, GALNT12, GALNT2, GALNT3, GALNT7, GAN, GANC,
GAPDH, GARS1, GART, GAS2L3, GAS6, GASK1B, GATA2, GATA3, GATAD2A,
GATAD2B, GATB, GBA, GBE1, GBGT1, GCAT, GCC1, GCC2, GCNT1, GCNT4,
GCSAM, GCSAML, GDAP1, GDE1, GDF11, GDI2, GDPD3, GEMIN7, GEN1, GET3,
GFI1, GFM1, GFM2, GFRA2, GGACT, GGCT, GGH, GGT7, GHITM, GHRL, GIGYF1,
GIMAP5, GIMAP7, GIMAP8, GINS2, GIPC1, GIPR, GK5, GKAP1, GLB1, GLB1L2,
GLCCI1, GLCE, GLDC, GLDN, GLE1, GLI1, GLI4, GLIPR1, GLMP, GLO1, GLRX,
GLRX3, GLS, GLTP, GLUD1, GLUD2, GM2A, GMCL1, GMDS, GMPPA, GMPR,
GNA15, GNAL, GNAO1, GNB5, GNG2, GNG5, GNL2, GNLY, GNPAT, GNPDA1,
GNPTAB, GNRH1, GNS, GOLGA4, GOLGA5, GOLGA6L10, GOLGA6L3, GOLGA6L4,
GOLGA6L9, GOLGA7B, GOLGA8A, GOLGA8B, GOLT1B, GORASP1, GOSR1, GOT2,
GPA33, GPALPP1, GPAT2, GPBAR1, GPD2, GPER1, GPI, GPM6B, GPN3, GPNMB,
GPR146, GPR153, GPR155, GPR171, GPR174, GPR18, GPR183, GPR3, GPR34, GPR68,
GPR75, GPRC5D, GPSM3, GPT2, GPX1, GPX4, GRAMD1C, GRB10, GRB2, GRHPR,
GRIN3B, GRINA, GRK2, GRK3, GRK5, GRM2, GRN, GSR, GSTM1, GSTM2, GSTM4,
GSTO1, GSTZ1, GTF2B, GTF2F2, GTF2H1, GTF3C6, GTPBP4, GTPBP8, GTSE1, GTSF1,
GUF1, GUSB, GXYLT1, GYG1, GYPE, GYS1, GZMA, GZMB, GZMH, GZMK, GZMM,
H1-0, H1-10, H1-4, H2AJ, H2AZ2, H2BC17, H2BC5, H3-3B, H4-16, H4C8, HAAO,
HABP4, HADHA, HADHB, HAGHL, HAT1, HAUS1, HAUS4, HAUS5, HAVCR2,
HCAR2, HCAR3, HCCS, HCN2, HCN3, HDAC8, HDAC9, HDC, HDGF, HDHD5, HDLBP,
HEATR4, HEATR9, HEBP1, HECTD2, HEG1, HELB, HERC1, HES4, HEXB, HEXD,
HFE, HGF, HIBADH, HIGD1A, HIGD2A, HINT2, HIP1R, HIRIP3, HIVEP2, HJURP, HK1,
HK2, HK3, HKDC1, HLA-DQA2, HLA-DRA, HLA-E, HLA-F, HLX, HM13, HMBS,
HMCES, HMGB2, HMGB3, HMGCL, HMGXB4, HMOX1, HMOX2, HNMT, HNRNPA0,
HNRNPAB, HNRNPDL, HNRNPF, HNRNPK, HOMEZ, HOPX, HOXB2, HOXB3,
HOXB4, HP, HPCAL1, HPCAL4, HPR, HPRT1, HPS5, HPSE, HRH4, HS1BP3,
HS3ST3B1, HSBP1, HSD17B10, HSD17B12, HSD17B4, HSF2, HSF4, HSPA4, HSPH1,
HTRA1, HUS1, HVCN1, HYAL2, IAH1, IBTK, ICA1, ID2, ID3, IDE, IDH1, IDH2, IDH3G,
IDS, IER2, IER5, IFH6, IFI27L1, IFI27L2, IFI35, IFI44, IFI6, IFIT5, IFITM3, IFNAR1,
IFNG, IFNGR1, IFNGR2, IFT122, IFT80, IGF1R, IGF2BP3, IGFBP2, IGFBP3, IGFBP7,
IGIP, IGLL5, IK, IKBKE, IKZF1, IKZF2, IKZF3, IKZF4, IL11RA, IL12RB1, IL15, IL15RA,
IL17RA, IL18, IL18BP, IL1B, IL1R2, IL21R, IL24, IL2RA, IL2RB, IL2RG, IL32, IL3RA,
IL5RA, IL7R, IMMT, IMPA2, IMPACT, IMPAD1, ING5, INIP, INKA1, INO80C, INO80E,
INPP1, INPP4A, INPP4B, INPP5D, INPP5E, INPPL1, INSR, INTS6, IP6K2, IPO7, IPP,
IQCN, IQSEC3, IRAK1, IRAK1BP1, IRAK2, IRAK3, IRF2BP1, IRF3, IRF5, IRF8,
ISG20L2, ISYNA1, ITFG1, ITGA1, ITGA7, ITGAM, ITGB2, ITGB7, ITGB8, ITK, ITM2A,
ITM2C, ITPKB, ITPKC, ITPR3, ITPRID2, ITPRIPL1, ITPRIPL2, ITSN1, JADE1, JADE2,
JADE3, JAG1, JAGN1, JAK1, JAK2, JAKMIP1, JAKMIP2, JAM3, JCHAIN, JDP2,
JKAMP, JMY, JPH4, JUN, JUNB, JUND, JUP, KANK2, KANK3, KANSL1, KANSL2,
KARS1, KAT6A, KAT6B, KATNBL1, KBTBD11, KBTBD4, KBTBD6, KCNA3, KCNC4,
KCND3, KCNE1, KCNE1B, KCNE3, KCNK13, KCNMA1, KCNMB1, KCNQ1, KCTD12,
KCTD13, KCTD3, KCTD5, KDELR1, KDELR2, KDM1B, KDM2A, KDM3A, KDM5A,
KDM6B, KEL, KIAA0100, KIAA0355, KIAA0586, KIAA0895L, KIAA0930, KIAA1143,
KIAA1257, KIAA1324, KIAA1328, KIAA1522, KIAA1671, KIAA1958, KIAA2026, KIF11,
KIF14, KIF15, KIF18A, KIF18B, KIF19, KIF20B, KIF21A, KIF21B, KIF22, KIF23,
KIF26A, KIF2A, KIF2C, KIF3A, KIF4A, KIF5C, KIFC1, KIFC2, KIFC3, KIR2DL1,
KIR2DL3, KIR2DS4, KIR3DL1, KIR3DL2, KIZ, KLC2, KLC4, KLF12, KLF16, KLF2,
KLF5, KLHDC2, KLHDC4, KLHDC8A, KLHL11, KLHL12, KLHL14, KLHL15, KLHL25,
KLHL28, KLHL3, KLHL42, KLHL8, KLRB1, KLRC1, KLRC4, KLRD1, KLRF1, KLRG1,
KMO, KMT2A, KMT2B, KMT2E, KMT5C, KNL1, KNSTRN, KPNA5, KPTN, KRBA1,
KRI1, KRR1, KRT23, KRT72, KRT73, KRTCAP3, KY, L3MBTL3, LACC1, LACTB,
LAIR1, LAMA2, LAMB2, LAMC1, LAMTOR2, LAMTOR4, LAMTOR5, LAP3,
LAPTM4B, LAT, LAT2, LAX1, LBH, LCK, LCNL1, LCORL, LCP1, LDB1, LDHA, LDLR,
LDLRAD3, LDOC1, LEMD3, LENG1, LENG8, LEPROT, LGALS1, LGALS12, LGALS3,
LGALS8, LGALS9, LGALS9B, LGR6, LHFPL2, LHFPL6, LIG1, LIG4, LILRA4, LILRA5,
LILRA6, LILRB4, LILRB5, LIM2, LIMD2, LIN54, LIN7C, LINC01410, LINC01579,
LINGO1, LIPA, LIPE, LITAF, LIXIL, LLGL1, LLGL2, LMAN2, LMBR1, LMBRIL,
LMF1, LMF2, LMNA, LMNB1, LMO2, LMO7, LMTK3, LNPEP, LONP2, LONRF3,
LOXHD1, LOXL2, LPCAT1, LPCAT3, LPCAT4, LPL, LPXN, LRATD2, LRFN1, LRFN3,
LRIF1, LRIG1, LRMDA, LRP1, LRP2BP, LRP3, LRP8, LRPAP1, LRRC14, LRRC23,
LRRC37B, LRRC41, LRRC45, LRRC56, LRRC59, LRRC8D, LRRK1, LRWD1, LSM10,
LSM6, LSS, LTA4H, LTB, LTBP3, LTBP4, LTBR, LTF, LUC7L, LUC7L3, LY6G5C,
LY75, LY86, LY9, LYRM7, LYSMD4, LYZ, LZTFL1, LZTS3, M1AP, M6PR,
MACROH2A1, MACROH2A2, MAEA, MAF, MAFB, MAFG, MAGED2, MAGEE1,
MAGEF1, MALAT1, MAML3, MAN1A2, MAN1B1, MAN1C1, MAN2A2, MAN2B1,
MANBA, MAOA, MAOB, MAP10, MAP11, MAP1LC3A, MAP2K1, MAP2K6, MAP3K10,
MAP3K12, MAP3K20, MAP3K21, MAP3K6, MAP3K7CL, MAP3K9, MAP4K1, MAP4K2,
MAP9, MAPK13, MAPK14, MAPK3, MAPK6, MAPK8, MAPKAPK3, MAPKBP1,
MAPRE1, MAPRE2, MARC1, MARCHF1, MARCHF2, MARCHF5, MARCHF9,
MARCKSL1, MARCO, MARS2, MARVELD1, MASP2, MAST3, MAT2A, MATK,
MATR3, MBD5, MBLAC2, MBOAT1, MBP, MBTPS2, MC1R, MCEMP1, MCF2L,
MCF2L2, MCFD2, MCM10, MCM2, MCM3AP-AS1, MCM4, MCMBP, MCOLN1,
MCR1P1, MCTP1, MCTS1, MDGA1, MDH1, MDH2, MDS2, ME1, ME2, MECP2, MED1,
MED10, MED15, MED17, MED19, MED20, MED21, MED30, MED31, MED6, MED8,
MEF2A, MEF2C, MEGF6, MELK, MELTF, MERTK, MESD, METRN, METTL14,
METTL22, METTL4, METTL7A, METTL9, MEX3C, MFGE8, MFSD1, MFSD14B,
MFSD2B, MFSD8, MGAT1, MGAT4A, MGME1, MGST1, MGST2, MGST3, MIB2,
MICA, MICAL2, MICB, MICU1, MID2, MIEF2, MIGA1, MIGA2, MILR1, MINDY2,
MINK1, MIOS, MIR23A, MIS18BP1, MITF, MKI67, MKKS, MLANA, MLC1, MLEC,
MLF1, MLKL, MLLT11, MLLT3, MLLT6, MLX, MLXIP, MME, MMP14, MMP17,
MMP19, MMP23B, MMP24, MMP28, MOAP1, MOB1B, MOB3C, MORC4, MORN3,
MPC1, MPDU1, MPEG1, MPHOSPH10, MPHOSPH8, MPND, MPP1, MPP6, MPRIP,
MPV17, MPV17L2, MPZL3, MR1, MRC1, MRI1, MRM2, MROH6, MRPL10, MRPL12,
MRPL13, MRPL15, MRPL16, MRPL18, MRPL19, MRPL23, MRPL28, MRPL3, MRPL33,
MRPL36, MRPL37, MRPL41, MRPL47, MRPL51, MRPL55, MRPL57, MRPL58, MRPL9,
MRPS10, MRPS15, MRPS16, MRPS18A, MRPS2, MRPS23, MRPS5, MRPS6, MRPS7,
MRRF, MRTFB, MS4A2, MS4A3, MS4A4A, MS4A4E, MS4A6A, MSANTD2, MSANTD3,
MSANTD4, MSH3, MSL1, MSR1, MSRB2, MT1E, MT1F, MT1X, MT2A, MT-ATP6,
MTCH2, MTERF2, MTF1, MTFR1, MTHFD1, MTHFD2, MTHFR, MTI F3, MTMR10,
MTMR14, MTMR6, MT-ND1, MT-ND2, MT-ND5, MT-ND6, MTPAP, MTR, MTX3,
MUC12, MUC16, MUC6, MUTYH, MVD, MVK, MX1, MXD3, MYBL1, MYBL2,
MYCBP, MYCT1, MYD88, MYDGF, MYEF2, MYH10, MYH11, MYH7B, MYL5, MYLIP,
MYO10, MYO1E, MY06, MYO7A, MYO9A, MYOF, MYPOP, MYZAP, MZB1, MZT1,
N4BP2L2, NAA20, NAA30, NAA80, NAALAD2, NACA2, NACC2, NAE1, NAGA,
NAGK, NAGLU, NAIP, NANS, NAP1L1, NAP1L2, NAP1L3, NAP1L5, NAPA, NAT8B,
NATD1, NAV1, NBEA, NBL1, NBPF3, NBR1, NCALD, NCAM1, NCAPG, NCAPH,
NCAPH2, NCBP2AS2, NCEH1, NCF2, NCK2, NCKAP1L, NCOA3, NCR1, NCR3,
NCR3LG1, NCSTN, NDRG2, NDUFA1, NDUFA11, NDUFA12, NDUFA2, NDUFA3,
NDUFA4, NDUFA6, NDUFA8, NDUFA9, NDUFAB1, NDUFAF1, NDUFAF3, NDUFB1,
NDUFB10, NDUFB3, NDUFB4, NDUFB5, NDUFB9, NDUFC1, NDUFS1, NDUFS2,
NDUFS3, NDUFS4, NDUFS5, NDUFS6, NDUFS7, NDUFV3, NECTIN1, NECTIN2,
NEDD4, NEIL1, NEK3, NEK4, NEK6, NEK7, NELL2, NEMF, NEO1, NET1, NETO2,
NEURL1, NFATC2, NFATC3, NFE2L3, NFE4, NFIA, NFIL3, NFS1, NHP2, NHSL1,
NIBAN2, NINL, NIP7, NIPA1, NIPA2, NIPAL2, NIPAL3, NIPSNAP2, NIPSNAP3A,
NKD1, NKG7, NKIRAS2, NLGN2, NLGN3, NLN, NLRC3, NLRC4, NLRP12, NLRP3,
NLRP6, NME1, NME2, NMNAT1, NMT2, NMUR1, NOCT, NOG, NOL10, NOL12, NOL3,
NOL4L, NOP10, NOP53, NOSIP, NOTCH1, NOTCH4, NOXA1, NPAT, NPC2, NPL,
NPR2, NPRL2, NPTXR, NR3C2, NREP, NRF1, NRG1, NRIP1, NRM, NRROS, NSDHL,
NSG1, NSMCE3, NSMF, NSUN2, NT5DC2, NT5DC3, NTRK1, NTSR1, NUAK1, NUBP1,
NUBP2, NUCB1, NUDT16, NUDT6, NUMA1, NUMBL, NUP37, NUP43, NUP58, NUP93,
NUSAP1, NUTF2, NUTM2F, NUTM2G, OAF, OAS1, OAS2, OAS3, OASL, OBSCN,
OCIAD2, OCLN, OCRL, ODF2L, OFD1, OGDH, OGFR, OLAH, OLFML2A, OLIG1,
OPA1, OPLAH, OPN3, OR2A4, OR2A7, OR2B6, ORAI1, ORAI2, ORAI3, ORC1, ORC2,
ORC5, ORMDL2, ORMDL3, OS9, OSBPL11, OSBPL5, OSBPL7, OSM, OSTC, OTOF,
OTUD3, OTUD7B, OTULINL, OXA1L, OXR1, P2RX4, P2RX7, P2RY1, P2RY10,
P2RY14, P2RY8, P3H4, P4HA1, P4HB, PABPC3, PACC1, PACS1, PACSIN1, PADI2,
PAFAH2, PAIP2B, PAK1IP1, PALLD, PANX1, PAPOLG, PAPSS1, PAQR4, PARD6B,
PARP10, PARP15, PARP16, PARP4, PARVG, PASK, PATJ, PATL2, PAX8, PAXX, PBX4,
PBXIP1, PCCB, PCDH9, PCED1A, PCF11, PCK2, PCMT1, PCNA, PCNX2, PCNX3,
PCOLCE, PCSK7, PCSK9, PCTP, PCYT2, PDCD10, PDCD4, PDCD6IP, PDCD7, PDE12,
PDE1B, PDE2A, PDE3B, PDE7A, PDGFC, PDGFD, PDHB, PDIA3, PDIA5, PDIA6, PDK2,
PDK4, PDP2, PDRG1, PDSS1, PDXK, PDZD2, PDZD4, PELP1, PEPD, PER1, PER3,
PET100, PEX1, PEX11G, PFDN6, PFKFB3, PFN2, PGA3, PGAM1, PGAM4, PGAP3,
PGBD2, PGD, PGGHG, PGK1, PGLS, PGLYRP1, PGM2, PGM2L1, PHAX, PHC1,
PHETA2, PHF1, PHF10, PHF13, PHF19, PHF20, PHKB, PHLDB2, PHLDB3, PHLPP2,
PHRF1, PI16, PI3, PICK1, PIGF, PIGL, PIGN, PIGS, PIK3C2B, PIK3CB, PIK3CD, PILRB,
PIM1, PIN1, PINK1, PIP5K1B, PITPNC1, PITPNM1, PITPNM2, PITRM1, PIWIL4, PJA1,
PJVK, PKIA, PKM, PKMYT1, PKN1, PKNOX1, PKP4, PLA2G12A, PLA2G15, PLA2G4A,
PLA2G6, PLA2G7, PLAA, PLAAT3, PLAU, PLBD1, PLCB3, PLCD1, PLCD3, PLCH2,
PLCL1, PLCXD2, PLD1, PLD3, PLD4, PLEKHA1, PLEKHA5, PLEKHB1, PLEKHB2,
PLEKHF1, PLEKHG2, PLEKHG3, PLEKHG5, PLEKHH2, PLEKHM2, PLGLB1, PLIN2,
PLIN3, PLK1, PLLP, PLOD1, PLP2, PLPBP, PLSCR1, PLXDC1, PLXDC2, PLXNA1,
PLXNA3, PLXNA4, PMAIP1, PMEL, PMVK, PNISR, PNKD, PNKP, PNN, PNP, PNPO,
PNRC1, PODN, PODXL, POFUT1, POFUT2, POLD3, POLDIP2, POLDIP3, POLE4,
POLQ, POLR2D, POLR2E, POLR2G, POLR2M, POLR3G, POLRMT, POMP, PON2,
POP7, POTEF, POTEI, POTEJ, POU2AF1, POU6F1, PPARD, PPARG, PPARGC1B,
PPCDC, PPCS, PPFIA3, PPFIBP2, PPIA, PPIB, PPIG, PPL, PPM1K, PPM1N, PPOX,
PPPICC, PPP1R12C, PPP1R13B, PPP1R15A, PPP1R16B, PPP1R2, PPP1R26, PPP1R35,
PPP1R37, PPP1R3D, PPP1R8, PPP2CB, PPP2R2B, PPP2R2D, PPP2R3B, PPP2R5C,
PPP3CC, PPP6C, PPT1, PRAF2, PRAG1, PRAM1, PRCP, PRDM1, PRDM11, PRDM8,
PRDX1, PRDX3, PRDX4, PRDX5, PRDX6, PREB, PRELID1, PREP, PRF1, PRKAA1,
PRKAB1, PRKACA, PRKACB, PRKAG1, PRKCH, PRKCQ, PRKCZ, PRKD2, PRKRIP1,
PRMT2, PRMT5, PROSER1, PRPF38A, PRPF38B, PRPF39, PRPF4, PRPF40B, PRR11,
PRR12, PRR14, PRR5, PRR5L, PRR7, PRRC2C, PRRG4, PRRT3, PRRT4, PRSS23,
PRSS33, PRSS53, PRSS57, PSAP, PSD, PSD3, PSD4, PSEN1, PSEN2, PSIP1, PSMA4,
PSMA5, PSMA6, PSMA7, PSMB1, PSMB2, PSMB3, PSMB5, PSMB6, PSMB7, PSMC1,
PSMD1, PSMD10, PSMD11, PSMD14, PSMD2, PSMD3, PSMD4, PSMD5, PSMD8,
PSMG1, PSMG2, PSPH, PSTK, PSTPIP2, PTBP1, PTBP2, PTCD1, PTCH1, PTDSS1,
PTGDR, PTGDR2, PTGDS, PTGER2, PTGER4, PTGFRN, PTGS2, PTK7, PTOV1,
PTP4A3, PTPA, PTPMT1, PTPN11, PTPN2, PTPN4, PTPN7, PTPN9, PTPRO, PTPRS,
PTRHD1, PTS, PTTG1, PTTG1IP, PUDP, PUM2, PURA, PUS7L, PVR, PXK, PXN,
PYCARD, PYGM, PYGO2, PYHIN1, PYM1, PZP, QRICH2, QSER1, QSOX1, QTRT2,
R3HDM1, RAB10, RAB11FIP3, RAB11FIP4, RAB11FIP5, RAB13, RAB15, RAB20,
RAB22A, RAB32, RAB34, RAB37, RAB39A, RAB39B, RAB43, RAB44, RAB6D, RAB7A,
RAB8A, RABEP2, RABGAP1L, RABL2B, RAC1, RACGAP1, RAD1, RAD23B,
RAD51AP1, RAD52, RAD9A, RADX, RALA, RALGDS, RAN, RANBP6, RAP2A, RAP2C,
RAPGEF5, RAPGEF6, RAPH1, RARG, RASA2, RASA3, RASAL3, RASGEF1B,
RASGRF2, RASGRP1, RASGRP2, RASSF1, RASSF2, RASSF5, RASSF7, RB1, RBBP6,
RBBP8, RBKS, RBL2, RBM14, RBM14-RBM4, RBM25, RBM34, RBM4, RBM45,
RBM48, RBMS2, RBP7, RBPMS, RBX1, RCAN1, RCAN3, RCBTB2, RCC1, RCC2, RCE1,
RCN2, RDH13, RDH14, RDH5, RDX, RECK, REEP4, REEP6, REM2, RENBP, RESF1,
REST, RETREG1, RETREG3, REX1BD, REXO1, RFC2, RFTN1, RFX1, RFXAP, RGL4,
RGMB, RGPD5, RGPD6, RGS1, RGS14, RGS16, RGS3, RGS6, RGS9, RHAG, RHBDD1,
RHBDF2, RHCE, RHEB, RHEBL1, RHO, RHOA, RHOBTB3, RHOC, RHOF, RHOQ,
RHOT1, RHOT2, RHOU, RHPN1, RIC1, RIC8B, RILPL1, RILPL2, RIMS3, RIN1, RIN2,
RINL, RIOK3, RIOX1, RMI1, RNASE1, RNASE2, RNASE3, RNASE6, RNF10, RNF103,
RNF126, RNF130, RNF135, RNF139, RNF144A, RNF144B, RNF165, RNF167, RNF175,
RNF181, RNF182, RNF216, RNF217, RNF41, RNF43, RNF44, RNF7, RNH1, RNPEP,
ROGDI, ROMO1, RORA, RPE, RPF1, RPGR, RPH3A, RPL13A, RPL17, RPL22L1,
RPL26L1, RPL3, RPN1, RPN2, RPRD2, RPS14, RPS27A, RPS27L, RPS29, RPS3, RPS5,
RPS6KA5, RPS6KC1, RPTOR, RPUSD2, RRAGA, RRAS, RRAS2, RRM2, RRP15,
RSAD1, RSAD2, RSBN1, RSBN1L, RSF1, RSPRY1, RSRP1, RTL6, RTL8C, RTN4,
RTRAF, RTTN, RUFY3, RUFY4, RUNDC1, RUNX3, RUSC2, RXFP2, RXFP4, RXRA,
RYK, RYR3, S100A10, S100A11, S100A12, S100A4, S100A6, S100A8, S100A9, S1PR1,
S1PR3, S1PR4, S1PR5, SACMIL, SAFB2, SALL2, SAMD10, SAMD3, SAMHD1,
SAMSN1, SAP30, SAP30L, SAPCD2, SAR1B, SARDH, SARM1, SASH1, SASS6, SAT2,
SBDS, SBF1, SBF2, SBK1, SCAI, SCAMP2, SCAP, SCAPER, SCARB1, SCARB2,
SCCPDH, SCD, SCD5, SCFD1, SCIMP, SCML4, SCNM1, SCP2, SCPEP1, SCR1B, SDC1,
SDC3, SDC4, SDCBP, SDCCAG8, SDE2, SDHB, SDHC, SDHD, SDK2, SEC11A, SEC23B,
SEC23IP, SEC24A, SEC24D, SEC31A, SEC61A1, SEC61A2, SEC61B, SEC61G,
SELENOF, SELENOM, SELENON, SELL, SEMA4A, SEMA4C, SEMA4F, SEMA6A,
SEMA6B, SEMA6C, SENP7, SEPHS2, SEPTIN1, SEPTIN10, SEPTIN2, SEPTIN4,
SEPTIN6, SEPTIN7, SEPTIN8, SEPTIN9, SERF1A, SERF2, SERHL2, SERP1, SERPINB1,
SERPINB10, SERPINB2, SERPINB8, SERPINB9, SERPINE1, SERPING1, SESTD1,
SETBP1, SETD1A, SETD1B, SETD3, SETD6, SETD7, SEZ6L, SF1, SF3A2, SF3B3, SFH,
SFMBT1, SFMBT2, SFSWAP, SFT2D1, SFXN4, SFXN5, SGCD, SGF29, SGIP1, SGK3,
SGMS1, SGMS2, SGO1, SGPP1, SGSH, SGSM1, SH2B1, SH2D1B, SH2D2A, SH2D3A,
SH2D3C, SH2D4A, SH3BGRL, SH3BGRL3, SH3BP1, SH3BP4, SH3PXD2B, SH3RF1,
SH3TC1, SH3YL1, SHCBP1, SHISA7, SHISAL2A, SHLD2, SHMT1, SHMT2, SHROOM1,
SHTN1, SIAE, SIAH2, SIDT1, SIGIRR, SIGLEC1, SIGLEC11, SIGLEC12, SIGLEC15,
SIGLEC16, SIGLEC9, SIKE1, SIL1, SIN3A, SIPA1, SIRPA, SIRPB2, SIRPD, SIVA1,
SKAP1, SKAP2, SKI, SKIV2L, SLAIN1, SLAMF1, SLAMF6, SLC11A1, SLC11A2,
SLC12A2, SLC13A4, SLC16A1, SLC16A11, SLC16A5, SLC16A6, SLC16A8, SLC19A2,
SLC1A3, SLC1A5, SLC1A7, SLC22A15, SLC22A16, SLC24A4, SLC25A11, SLC25A24,
SLC25A26, SLC25A29, SLC25A30, SLC25A38, SLC25A40, SLC25A42, SLC25A43,
SLC25A45, SLC25A5, SLC25A53, SLC25A6, SLC26A11, SLC26A6, SLC27A1, SLC27A3,
SLC29A2, SLC29A3, SLC2A4, SLC2A4RG, SLC2A5, SLC2A9, SLC30A1, SLC30A4,
SLC31A1, SLC31A2, SLC33A1, SLC35A1, SLC35A3, SLC35A5, SLC35B1, SLC35B3,
SLC35D1, SLC35D2, SLC35E2B, SLC35F6, SLC36A1, SLC36A4, SLC37A2, SLC38A1,
SLC38A2, SLC38A5, SLC38A7, SLC39A1, SLC39A11, SLC39A13, SLC39A7, SLC39A9,
SLC3A2, SLC41A1, SLC43A1, SLC43A3, SLC44A1, SLC45A3, SLC45A4, SLC46A2,
SLC49A3, SLC4A11, SLC4A2, SLC4A4, SLC4A5, SLC4A7, SLC50A1, SLC5A3,
SLC6A12, SLC6A16, SLC7A7, SLC9A3R1, SLC9A9, SLC9B2, SLCO4A1, SLF1, SLFN11,
SLFN12, SLFN12L, SLIRP, SLITRK4, SLPI, SMAD1, SMAD3, SMAD4, SMAD7,
SMARCAL1, SMARCD3, SMCO4, SMG7, SMIM10L2A, SMIM12, SMIM15, SMIM24,
SMIM27, SMIM3, SMIM4, SMPD3, SMS, SMTN, SMU1, SMURF2, SMYD3, SMYD4,
SNAPIN, SND1, SNF8, SNPH, SNRK, SNRNP25, SNRNP48, SNRNP70, SNRPB2,
SNRPC, SNRPD2, SNRPD3, SNRPG, SNTA1, SNTB1, SNUPN, SNURF, SNX1, SNX10,
SNX12, SNX17, SNX19, SNX2, SNX25, SNX29, SNX3, SNX30, SNX33, SNX5, SNX8,
SOAT1, SOCS2, SOCS6, SORBS3, SORD, SORL1, SORT1, SOWAHC, SOWAHD,
SOX12, SOX13, SOX8, SP4, SPAG16, SPAG5, SPATA33, SPATA5, SPATS2, SPATS2L,
SPCS1, SPCS2, SPDYE3, SPECC1, SPEF2, SPG21, SPG7, SPHK1, SPIDR, SPINDOC,
SPINT1, SPINT2, SPN, SPNS3, SPOCK1, SPOCK2, SPON1, SPON2, SPOUT1, SPR,
SPRN, SPRY2, SPRYD3, SPSB1, SPSB2, SPTAN1, SPTB, SPTBN5, SPTLC1, SPTLC2,
SPTSSA, SQLE, SQOR, SRA1, SRD5A1, SRD5A3, SRGAP1, SRGAP2, SRGAP2B,
SRGAP2C, SRGAP3, SRP54, SRP72, SRP9, SRPK2, SRPK3, SRPRA, SRRT, SRSF1,
SRSF11, SRSF5, SRSF7, SSBP3, SSBP4, SSR1, SSR3, SSR4, ST14, ST3GAL3, ST3GAL4,
ST6GAL1, ST6GALNAC3, ST6GALNAC4, ST8SIA1, ST8SIA6, STAB1, STAC3,
STAG3L3, STARD13, STARD3NL, STARD8, STARD9, STAT1, STAT2, STAT4,
STAT5B, STEAP3, STIL, STIM2, STING1, STK10, STK26, STK3, STK39, STMN3,
STMP1, STON1, STS, STT3A, STT3B, STX10, STX12, STX4, STYXL1, SUCLA2,
SUCLG1, SULT1A1, SULT1A2, SUMF1, SUMO3, SUN2, SUOX, SURF4, SUSD3, SV2A,
SVIP, SWAP70, SYDE2, SYK, SYN2, SYNE1, SYNE2, SYNE3, SYNGAP1, SYNGR2,
SYNJ2, SYNM, SYNRG, SYTL1, SYTL2, SYTL3, TAF10, TAF12, TAF3, TAF4, TAF7,
TAF9, TAGAP, TAGLN2, TAL1, TALDO1, TANC2, TAOK2, TARS1, TARS3, TBC1D1,
TBC1D10A, TBC1D10C, TBC1D13, TBC1D16, TBC1D17, TBC1D19, TBC1D2,
TBC1D25, TBC1D31, TBC1D3B, TBC1D3D, TBC1D3F, TBC1D3H, TBC1D3I, TBC1D3L,
TBC1D4, TBC1D5, TBC1D8, TBCC, TBKBP1, TBL2, TBX21, TBXAS1, TC2N, TCAF1,
TCAF2, TCAIM, TCEA2, TCEA3, TCEAL4, TCF20, TCF7, TCN1, TCN2, TCOF1,
TCP11L2, TCTEX1D4, TCTN2, TDRD9, TEC, TECR, TEDC1, TEF, TEFM, TENT4B,
TESPA1, TET3, TEX2, TEX52, TFCP2, TFDP1, TFDP2, TFEC, TFG, TFRC, TGFB3,
TGFBR1, TGFBR2, TGFBR3, TGM2, THAP1, THAP5, THAP7, THBS1, THBS4, THEM4,
THG1L, THOC6, THOC7, THUMPD1, THUMPD2, THUMPD3, TIAM2, TIGAR, TIGD1,
TIGD2, TIGD3, TIGD5, TIMM13, TIMM23, TIMM8B, TIMMDC1, TIMP1, TIMP2,
TIMP4, TJP3, TK1, TK2, TKFC, TKT, TKTL1, TLE2, TLN2, TLR2, TLR4, TLR5, TM2D3,
TM7SF3, TM9SF2, TM9SF4, TMA16, TMBIM6, TMC6, TMC8, TMCC2, TMCC3,
TMCO1, TMED8, TMED9, TMEM106A, TMEM11, TMEM115, TMEM116, TMEM120B,
TMEM121B, TMEM131, TMEM134, TMEM144, TMEM147, TMEM14A, TMEM14C,
TMEM150B, TMEM161B, TMEM165, TMEM167A, TMEM170B, TMEM179B,
TMEM181, TMEM184A, TMEM184B, TMEM185B, TMEM204, TMEM205, TMEM208,
TMEM218, TMEM219, TMEM222, TMEM230, TMEM243, TMEM25, TMEM254,
TMEM255A, TMEM255B, TMEM258, TMEM259, TMEM263, TMEM268, TMEM272,
TMEM273, TMEM38A, TMEM39A, TMEM39B, TMEM41B, TMEM45A, TMEM45B,
TMEM50B, TMEM60, TMEM62, TMEM63A, TMEM63C, TMEM64, TMEM65, TMEM67,
TMEM69, TMEM70, TMEM8B, TMEM9B, TMIGD2, TMIGD3, TMLHE, TMSB15B,
TMTC1, TMTC2, TMTC3, TMUB1, TMX1, TMX2, TNFAIP8L2, TNFRSF10B,
TNFRSF10C, TNFRSF10D, TNFRSF17, TNFRSF18, TNFRSF25, TNFSF10, TNFSF13,
TNFSF8, TNIK, TNNT3, TNRC6B, TNRC6C, TNS2, TNS3, TOB1, TOB2, TOE1,
TOGARAM2, TOLLIP, TOM1L2, TOMM22, TOMM40L, TOP1MT, TOP2A, TOPORS,
TOR1A, TOR3A, TOR4A, TOX, TOX4, TP53BP1, TP53BP2, TP53I13, TP53I3, TP53TG5,
TPCN2, TPI1, TPK1, TPM2, TPM4, TPMT, TPP1, TPPP, TPPP3, TPRG1, TPRG1L, TPST1,
TPX2, TRA2A, TRA2B, TRABD2A, TRADD, TRAF1, TRAF3IP3, TRAF4, TRAF7,
TRAFD1, TRANK1, TRAP1, TRAPPC10, TRAPPC11, TRAPPC2, TRAPPC3, TRAPPC4,
TRAPPC6A, TRAT1, TREM1, TREML2, TRERF1, TREX2, TRGC1, TRIM23, TRIM28,
TRIM32, TRIM33, TRIM46, TRIM56, TRIM59, TRIM62, TRIM7, TRIM71, TRIM73,
TRIP12, TRIP13, TRIQK, TRIR, TRMT10C, TRMT11, TRMT13, TRMT2B, TRMT6,
TRPC1, TRPC6, TRPM2, TRPS1, TSC22D4, TSEN54, TSG101, TSHR, TSHZ1, TSHZ2,
TSPAN13, TSPAN18, TSPAN2, TSPAN3, TSPAN32, TSPAN4, TSPO, TSPOAP1,
TSPYL1, TSPYL2, TSPYL4, TSSK6, TSTA3, TSTD1, TTC14, TTC16, TTC17, TTC22,
TTC3, TTC33, TTC38, TTC39B, TTC9, TTK, TTL, TTPAL, TUBA1A, TUBA1B, TUBB,
TUBB2A, TUBE1, TUBG1, TUBGCP2, TUBGCP3, TUBGCP6, TUFM, TUT4, TVP23C,
TWF2, TWSG1, TXLNG, TXN, TXN2, TXNDC11, TXNDC15, TXNDC17, TXNDC5,
TXNL1, TXNL4B, TXNRD2, TYMP, TYMS, TYSND1, UACA, UAP1L1, UBA1, UBA3,
UBAP1, UBAP1L, UBE2C, UBE2E1, UBE2E2, UBE2H, UBE2J1, UBE2K, UBE2L3,
UBE2L6, UBE2S, UBE3D, UBL3, UBQLN1, UBQLN2, UBQLN4, UBTD1, UBTD2,
UBTF, UBXN11, UBXN8, UCP3, UGCG, UGGT2, UNC119B, UNC13B, UNC50,
UNC5CL, UNC93B1, UNK, UNKL, UPF3B, UPP1, UPRT, UQCR10, UQCR11, UQCRC1,
UQCRC2, UQCRFS1, UQCRH, UQCRHL, UQCRQ, URI1, UROD, USP11, USP18, USP20,
USP21, USP39, USP41, USP42, USP51, USP54, USP6NL, USP9Y, UST, UTP11, UTP23,
UVRAG, UVSSA, VAMP1, VAMP2, VAMP3, VAMP4, VAMP5, VAMP7, VAMP8,
VAPA, VARS1, VASH1, VCAN, VDAC1, VDAC2, VDR, VEGFB, VENTX, VIM,
VIPAS39, VNN1, VPS11, VPS25, VPS26B, VPS26C, VPS35, VPS35L, VPS37A, VPS45,
VPS54, VRK2, VSIG1, VSIG10L, VSIG4, VTA1, VTI1B, VXN, WARS2, WASF1,
WASHC2A, WASHC2C, WASHC4, WASHC5, WASL, WDFY1, WDR12, WDR18,
WDR27, WDR35, WDR41, WDR54, WDR60, WDR61, WDR62, WDSUB1, WFDC1,
WHAMM, WHRN, WIPF1, WNT10B, WNT11, WNT7A, WSB1, WWC2, WWP1, XCL1,
XCL2, XK, XKR6, XKR8, XPC, XPNPEP1, XPNPEP2, XPO6, XPO7, XRCC2, XRCC3,
XRCC4, XRCC5, XRN2, XRRA1, XXYLT1, XYLT1, YAF2, YEATS2, YES1, YIF1A,
YIF1B, YIPF1, YIPF2, YIPF6, YPEL1, YPEL2, YPEL4, YPEL5, YRDC, YWHAE,
YWHAG, YWHAH, YY1, YY1AP1, ZAP70, ZBED2, ZBTB1, ZBTB10, ZBTB14, ZBTB16,
ZBTB18, ZBTB20, ZBTB25, ZBTB32, ZBTB38, ZBTB4, ZBTB40, ZBTB43, ZBTB44,
ZBTB46, ZBTB7A, ZC3H12B, ZC3H12C, ZC3H12D, ZC3H13, ZC3H6, ZC3H8,
ZC3HAV1, ZCCHC14, ZCCHC18, ZCCHC2, ZCCHC3, ZDHHC11, ZDHHC11B,
ZDHHC12, ZDHHC18, ZDHHC20, ZDHHC3, ZDHHC5, ZDHHC8, ZEB1, ZFAND5, ZFP1,
ZFP14, ZFP30, ZFP41, ZFP57, ZFP62, ZFP82, ZFP91, ZFPM1, ZFYVE26, ZFYVE27,
ZFYVE28, ZHX2, ZMAT5, ZMPSTE24, ZMYM1, ZMYM4, ZMYM5, ZMYND11,
ZMYND19, ZNF100, ZNF106, ZNF107, ZNF12, ZNF121, ZNF131, ZNF136, ZNF138,
ZNF148, ZNF160, ZNF169, ZNF175, ZNF182, ZNF200, ZNF213, ZNF236, ZNF248,
ZNF250, ZNF251, ZNF26, ZNF260, ZNF264, ZNF266, ZNF274, ZNF276, ZNF286A,
ZNF287, ZNF292, ZNF32, ZNF324, ZNF329, ZNF330, ZNF335, ZNF33B, ZNF345,
ZNF350, ZNF365, ZNF366, ZNF382, ZNF383, ZNF394, ZNF414, ZNF428, ZNF429,
ZNF430, ZNF439, ZNF44, ZNF441, ZNF446, ZNF461, ZNF469, ZNF48, ZNF483, ZNF486,
ZNF487, ZNF506, ZNF507, ZNF517, ZNF519, ZNF529, ZNF540, ZNF548, ZNF549,
ZNF552, ZNF562, ZNF566, ZNF567, ZNF568, ZNF569, ZNF570, ZNF573, ZNF574,
ZNF575, ZNF577, ZNF579, ZNF587, ZNF599, ZNF600, ZNF608, ZNF611, ZNF622,
ZNF644, ZNF652, ZNF665, ZNF674, ZNF680, ZNF688, ZNF697, ZNF699, ZNF703,
ZNF707, ZNF71, ZNF720, ZNF736, ZNF737, ZNF74, ZNF770, ZNF782, ZNF783, ZNF789,
ZNF793, ZNF80, ZNF827, ZNF83, ZNF830, ZNF831, ZNF836, ZNF850, ZNF853, ZNF862,
ZNF865, ZNF91, ZNF92, ZNHIT6, ZNRD2, ZRANB2, ZSCAN18, ZSWIM5, ZSWIM6,
ZSWIM9, ZW10, ZWINT, ZXDA, ZXDB
Lung-CoV2 to Lung-CTL (2,220 DEGs in Lung)
A2M, AAK1, ABCC3, ABCF1, ABB, ABLIM1, ABLIM3, ABR, ABT1, ABTB1, ACAD8,
ACADVL, ACO2, ACOD1, ACOT1, ACP2, ACSL1, ACSS1, ACTB, ACTL6A, ACTN4,
ACTR1B, ACVR1, ACVRL1, ADA2, ADAM15, ADAM17, ADAMDEC1, ADAMTS1,
ADAMTS13, ADAMTS2, ADAMTS9, ADAMTSL3, ADAP1, ADCY4, ADCY6,
ADCYAP1, ADD1, ADGRD1, ADGRE1, ADGRE5, ADGRG3, ADGRL2, ADH1B,
ADIPOR2, ADNP2, ADRA2A, AEBP1, AEBP2, AEN, AFAP1L1, AFDN, AFF4, AFG3L2,
AFMID, AGAP3, AGAP4, AGAP6, AGPAT2, AHNAK, AIF1L, AIM2, AIP, AKAP1,
AKAP13, AKAP17A, AKNA, AKR1B10, AKR1B15, AKT3, ALAS1, ALDH3A1, ALDOC,
ALKBH5, ALKBH6, ALOX5AP, ALPK1, ALPL, ALS2CL, AMBRA1, AMFR, AMOTL1,
ANAPC11, ANGEL1, ANGPTL2, ANGPTL6, ANKDD1A, ANKRD10, ANKRD22,
ANKRD54, ANKS1A, ANKZF1, ANO6, ANP32A, ANP32D, ANXA2R, ANXA6, AOC3,
AP2A2, APBB1IP, APH1A, APOBEC3A, APOL6, AQP1, AQP9, ARAP3, ARC, ARF5,
ARFGAP1, ARFRP1, ARHGAP10, ARHGAP4, ARHGAP9, ARHGDIB, ARHGEF15,
ARHGEF25, ARHGEF7, ARL13B, ARL5B, ARMC5, ARMC6, ARPC5L, ARRB2,
ARRDC2, ARSL, ASB1, ASB13, ASB6, ASB8, ASCL4, ASL, ASMTL, ASXL1, ASXL2,
ATAD3A, ATAD3B, ATF4, ATF6B, ATG101, ATG16L1, ATG4B, ATP1A1, ATP1B4,
ATP2A3, ATP5MF, ATP6V0A1, ATP6V0A2, ATP6V0D1, ATP7B, ATRX, ATXN7L3,
AUP1, B4GALT2, B4GALT3, B4GALT7, BAALC, BAG3, BAG6, BAIAP2, BAIAP2L1,
BANF1, BANP, BAP1, BASP1, BAZ2A, BBOF1, BBS1, BBS4, BCAR1, BCAT1, BCAT2,
BCL2A1, BCL2L14, BCL2L2, BCL3, BCOR, BEST1, BHLHE40, BIN1, BIN2, BIN3,
BLCAP, BLOC1S1, BMS1, BRD9, BSDC1, BST1, BTBD17, BTBD19, BTG2, BTN2A2,
BTNL9, BUD13, BUD23, BYSL, C12orf10, C12orf57, C14orf132, C15orf48, C19orf84,
C1orf194, C1orf216, C1QB, C1QC, C1QTNF1, C2, C20orf203, C20orf27, C2CD2, C3AR1,
C3orf86, C4orB, C4orf50, C5AR1, C6, C6orf201, C6orf47, C7orf25, C7orf50, C8A,
C8orB3, C8orf58, CA1, CA12, CA8, CAB39L, CACNA1A, CACNA1C, CACNA1E,
CACNG6, CALHM2, CALR, CAMP, CANT1, CAP1, CAPRIN2, CAPZA1, CARD16,
CARD17, CASP1, CASP4, CASP5, CAVIN1, CAVIN3, CBFA2T2, CBX4, CBX7, CCAR1,
CCDC124, CCDC130, CCDC137, CCDC185, CCDC186, CCDC194, CCDC38, CCDC57,
CCDC60, CCDC69, CCDC71L, CCDC84, CCDC86, CCDC9, CCL11, CCL15, CCL18,
CCL19, CCL2, CCL3, CCL3L1, CCL4, CCL4L2, CCL7, CCL8, CCM2, CCN2, CCN5,
CCND1, CCND3, CCNL2, CCPG1, CCR1, CCR7, CCRL2, CCT4, CCT5, CCT8, CD164,
CD2BP2, CD320, CD34, CD37, CD38, CD4, CD53, CD55, CD69, CD74, CD82, CD8A,
CD9, CDA, CDC37, CDC42BPB, CDCA7, CDH11, CDH2, CDH5, CDHR5, CDK10,
CDK18, CDK4, CDK9, CDKN2AIP, CEACAM1, CEACAM3, CEACAM6, CEACAM8,
CELA3B, CEMIP2, CENPC, CENPT, CEP68, CERK, CERS5, CERS6, CERT1, CETN1,
CFAP20, CFAP300, CFAP36, CFAP77, CFL1, CFLAR, CGRRF1, CHD2, CHI3L1, CHI3L2,
CHID1, CHIT1, CHMP1A, CHRAC1, CHROMR, CIB1, CIRI, CIRBP, CKB, CKS1B, CLC,
CLCN6, CLDN2, CLDN4, CLDN5, CLEC12A, CLEC12B, CLEC1A, CLEC4A, CLEC4D,
CLEC4E, CLEC7A, CLIC3, CLIP4, CLPS, CLPTM1L, CMC2, CMPK2, CMTM2, CNOT1,
CNST, CNTN2, CNTROB, COASY, COG1, COG4, COL12A1, COL16A1, COL2A1,
COL9A2, COLGALT1, COPZ2, CORO1C, COX4I1, CPA5, CPAMD8, CPE, CPLX2,
CPSF7, CPXM2, CR1, CRABP1, CRAMP1, CRCT1, CREB3L2, CREBZF, CRELD2,
CRIP2, CRISP3, CRTC2, CRY2, CRYAB, CSF2RB, CSF3R, CSNK1A1, CSNK1D,
CSNK2A2, CSNK2B, CSPG4, CSRNP1, CST7, CSTA, CTCF, CTDP1, CTDSP2, CTF1,
CTPS1, CTSE, CTSL, CTSS, CTTN, CUL1, CUL9, CWC22, CWF19L2, CXCL10,
CXCL11, CXCL16, CXCL17, CXCR2, CXorf21, CXorf40A, CXorf40B, CXXC5, CYB5R1,
CYBB, CYBRD1, CYC1, CYHR1, CYP19A1, CYP1A1, CYP24A1, CYP2J2, CYP3A5,
CYP4F3, CYP7B1, CYSTM1, CYTH1, CYTIP, DAAM1, DAAM2, DAB2IP, DAPK3,
DAPP1, DAXX, DBF4B, DBNDD1, DCAF12L1, DCAF13, DCAF5, DCDC2C, DCHS1,
DCUN1D3, DCUN1D4, DDB1, DDB2, DDR2, DDT, DDTL, DDX23, DDX39A, DDX42,
DDX5, DDX56, DDX58, DDX60, DDX60L, DENND2A, DENND3, DEPP1, DES, DGKD,
DGKE, DHDDS, DHPS, DHRS9, DHX29, DHX32, DHX9, DIAPH1, DIDO1, DIP2A,
DIPK2B, DISP1, DKK3, DLG4, DLK1, DLL1, DLL4, DLST, DLX2, DMBT1, DMRT1,
DMRTB1, DNAAF1, DNAAF3, DNAH7, DNAJB2, DNAJC11, DNAJC7, DNASE1,
DNMT1, DNPEP, DNTTIP1, DOCK1, DOCK6, DOCK9, DOK2, DOK3, DOK4, DPH1,
DPH2, DPH7, DPM3, DPP7, DPT, DPYSL3, DRD2, DST, DTX3, DTX4, DUSP1, DUSP10,
DUSP7, DYNLT1, DYRK2, DYRK3, E2F4, EBLN1, EBNA1BP2, ECE1, EDC3, EDC4,
EDNRA, EED, EEF2, EFEMP2, EFL1, EFNA1, EFNB1, EGF, EGFL7, EGR1, EGR2,
EIF1B, EIF2AK2, EIF2AK4, EIF2B4, EIF2B5, EIF2D, EIF3B, EIF3D, EIF3G, EIF3L,
EIF4A1, EIF4E2, EIF4G1, ELAC2, ELP1, EMC9, EMP1, EMP2, EN2, ENDOD1, ENG,
ENKD1, ENO2, ENOSF1, ENTPD6, ENTR1, EOMES, EPAS1, EPB41L1, EPC1, EPHA2,
EPHA7, EPHB4, EPN2, EPOP, EPSTI1, ERBB2, ERCC1, ERRFI1, ESF1, ETS1, ETV1,
EVI2A, EVI2B, EVI5, EVL, EXOC2, EXOC3, EXOSC7, EXOSC8, EZR, F2RL3, F5, F8A1,
F8A3, FABP6, FAM107A, FAM110D, FAM118A, FAM126A, FAM131A, FAM13B,
FAM151B, FAM160A2, FAM168B, FAM174B, FAM174C, FAM177B, FAM193A,
FAM199X, FAM207A, FAM20B, FAM20C, FAM214B, FAM234A, FAM32A, FAM3A,
FAM53B, FAM71D, FAM72A, FAM83G, FAM8A1, FAM9A, FAR2, FARS2, FARSA,
FASTK, FASTKD2, FAXDC2, FBH1, FBP1, FBXO31, FBXO39, FBXO9, FBXW4,
FBXW5, FCER1G, FCGR1A, FCGR2A, FCGR3A, FCGR3B, FCN1, FDCSP, FDPS, FER,
FES, FFAR2, FGD4, FGD5, FGFR1, FGG, FGL1, FGL2, FGR, FHL1, FHL3, FHOD1, FIBP,
FIS1, FKBP1A, FLAD1, FLG, FLG2, FLNA, FLNC, FLT1, FLT4, FLYWCH2, FNBP4,
FOS, FOSB, FOXD3, FOXD4L1, FOXJ2, FOXK2, FOXN3, FOXO4, FOXP2, FPR1, FPR2,
FREM2, FRMD6, FRYL, FXYD6, FYB1, FZD4, G6PD, GAB4, GABRA5, GADD45A,
GADD45B, GADD45G, GAK, GAL, GALNT15, GALNT18, GAN, GAPT, GAS5, GAS6,
GATA2, GATD3A, GBA2, GBP1, GBP3, GBP4, GBP5, GCA, GCFC2, GCH1, GCNT3,
GDPD5, GEM, GFOD1, GGA1, GGA2, GGA3, GIMAP1, GIMAP6, GIMAP7, GIPC1,
GJA4, GJA5, GK, GKN2, GLDN, GLG1, GLRX, GLT8D1, GMFG, GMPPA, GMPR,
GNA11, GNA12, GNG11, GNG2, GNG5, GNL1, GNL2, GNLY, GOLGA3, GOLGA6L4,
GOLGA8A, GOLGA8B, GON4L, GPAT4, GPD1L, GPM6B, GPN3, GPR141, GPR37L1,
GPR65, GPR68, GPR84, GPRC5B, GPSM3, GPX1, GPX2, GRB10, GRB7, GRPEL2,
GRSF1, GRWD1, GSDMD, GSK3A, GSN, GSS, GSTO1, GTF2B, GTF3C5, GTPBP4,
GTPBP6, GTSF1, GUCD1, GUCY1A2, GUK1, H19, H2AC18, H2AC19, H2AC20, H2AC6,
H2AJ, H2BC18, H2BC21, H2BC4, H2BC5, H2BC7, H4C8, HABP4, HAMP, HARS2,
HAUS2, HBA1, HBA2, HBB, HBD, HBG1, HBS1L, HCAR1, HCAR2, HCFC1R1, HCK,
HCST, HDAC3, HDAC7, HDLBP, HECTD1, HELB, HELZ2, HERC2, HERC5, HERC6,
HERPUD1, HES1, HESX1, HEXA, HEXB, HEYL, HIF3A, HINFP, HIPK1, HIPK2, HJURP,
HLA-A, HLA-B, HLA-DQA1, HLA-DQB1, HLA-DRB1, HLA-DRB5, HMCES, HMGCS1,
HMGN3, HMGXB3, HMOX1, HNRNPAB, HNRNPH1, HNRNPM, HOXA5, HOXB1, HP,
HPGDS, HPR, HPS4, HS1BP3, HS3ST3A1, HSDL1, HSFX1, HSFX2, HSH2D, HSPA12B,
HSPA2, HSPA6, HSPA8, HSPB6, HSPB7, HTR1A, HTRA3, HYAL1, HYAL2, HYOU1,
ICAM2, ID2, ID3, IDH2, IDO1, IER2, IER3, IER5L, IFFO1, IFI44, IFI44L, IFI6, IFIH1,
IFITI, IFITIB, IFIT2, IFIT3, IFIT5, IFITM1, IFITM2, IFITM3, IFNA10, IFNA17, IFNA21,
IFNA4, IFNA6, IFNL1, IFT43, IGFBP1, IGFLR1, IGLL1, IGLL5, IGSF3, IGSF6, IL12B,
IL17C, IL18RAP, ILIA, IL1B, IL1R2, ILIRN, IL27, IL2RG, IL4R, ILF3, ILKAP, ILRUN,
ILVBL, IMPDH2, INAFM1, ING2, ING5, INMT, INO80E, INPP1, INPP5A, INPP5K,
INSM1, INSYN2B, INTS10, INTS11, INTS3, IPO8, IQCM, IRF2, IRF7, ISG15, ISG20L2,
IST1, ITGA10, ITGA5, ITGA8, ITGB3, ITGB8, ITM2A, ITM2C, ITPA, ITPKB, ITPKC,
ITPR3, ITPRIP, JADE1, JAG1, JAK3, JAM3, JCAD, JCHAIN, JMJD6, JOSD2, JUN, JUND,
KANK2, KAT5, KCNE1, KCNEIB, KCNJ2, KCNK17, KCNK3, KCTD10, KCTD11,
KCTD12, KCTD2, KCTD20, KCTD3, KDM1A, KDM3A, KHDRBS3, KIAA0100,
KIAA0355, KIAA0513, KIAA0825, KIAA0930, KIAA1671, KIF1C, KIF22, KIF3B, KIFC2,
KLF10, KLF11, KLF13, KLF4, KLHDC3, KLHDC4, KLHDC7B, KLHL21, KLHL3,
KLHL42, KMT2A, KMT5A, KNDC1, KPRP, KRI1, KRIT1, KRT17, KRT19, KRT72,
KRT81, KSR1, LAGE3, LAMA2, LAMA4, LAMB1, LAMC3, LAMP3, LAMTOR4,
LARGE1, LARP6, LAS1L, LASP1, LBH, LBP, LCE1C, LCN2, LCP1, LCP2, LDB1,
LDLRAP1, LENG8, LEPR, LGALS9, LGALS9B, LGALS9C, LGI1, LGI4, LHFPL6, LIF,
LIFR, LILRA1, LILRA5, LILRA6, LILRB2, LILRB3, LILRB5, LIPA, LITAF, LMANIL,
LMAN2L, LMBR1L, LMNA, LMNB1, LMO2, LMOD1, LONP1, LOR, LPAR6, LPCAT4,
LRATD2, LRFN1, LRFN3, LRIG1, LRPAP1, LRRC1, LRRC15, LRRC32, LRRC47,
LRRC74A, LRRFIP1, LRRK2, LRRTM1, LSM7, LSS, LST1, LTF, LTO1, LUC7L,
LUC7L3, LUZP1, LY6E, LY86, LY96, LYN, LYPLA1, LYRM4, LYVE1, LZTR1, LZTS1,
LZTS2, MACC1, MADD, MAF1, MAFF, MAFG, MAFK, MAGEB16, MAN1B1,
MAN2C1, MAP2K3, MAP2K6, MAP2K7, MAP3K14, MAP3K6, MAP3K7CL, MAP4,
MAP4K5, MAPK11, MAPK9, MAPKAPK2, MARCHF2, MARCKS, MARK3, MARS1,
MAST2, MAT2A, MBD3, MBIP, MCAM, MCF2L, MCM3AP, MCM7, MCTP2, MCTS1,
MDC1, MDGA2, MDH1B, ME3, MED11, MED15, MED18, MED24, MEDAG, MEF2D,
MEIS1, MEPCE, METTL17, METTL3, METTL7A, METTL7B, MFAP1, MFGE8, MFN2,
MFNG, MFSD6, MGAM, MGAT5B, MGLL, MICAL1, MICB, MIER1, MIF4GD, MINK1,
MLLT10, MLNR, MLXIP, MMAB, MMP1, MMP10, MMP12, MMP14, MMP25, MMP28,
MMP8, MNDA, MOAP1, MOB2, MON2, MORC2, MPHOSPH10, MPHOSPH8, MPLKIP,
MPPED1, MPRIP, MPV17, MPZL1, MRFAP1L1, MRNIP, MRPL11, MRPL28, MRPL44,
MRPL55, MRPS2, MRTFA, MS4A15, MS4A3, MSTO1, MTA2, MTA3, MTCH1, MT-CO1,
MT-CO2, MT-CO3, MTFR1L, MTHFR, MT1F3, MTLN, MTURN, MTX1, MUC5AC,
MUL1, MVP, MX1, MX2, MXD1, MXD4, MXRA7, MYADM, MYBBP1A, MYC,
MYCBP2, MYCT1, MYL1, MYO15B, MYO1D, MYO1G, MYO9B, MYOM2, MYOZ2,
MYZAP, MZB1, MZT2B, NAA10, NAA35, NAA38, NAA60, NAB2, NADSYN1, NAP1L4,
NASP, NAT10, NAT9, NBL1, NBN, NBPF26, NCALD, NCAPH2, NCBP2AS2, NCF4,
NCKIPSD, NCR1, NDNF, NDRG1, NDRG2, NDST1, NDST2, NDUFA4L2, NDUFAB1,
NDUFS2, NDUFS6, NDUFS8, NDUFV1, NEBL, NECAP1, NEIL2, NEK7, NELFA,
NELFB, NELFE, NEMP2, NES, NETO1, NEXN, NF2, NFATC2, NFATC4, NFKBID,
NFRKB, NFX1, NIBAN2, NIPAL3, NISCH, NKD1, NKPD1, NLE1, NLRP1, NME1, NMI,
NMRAL1, NOC2L, NOD1, NOL4, NOL8, NOP10, NOP16, NOP53, NOP56, NOP9,
NORAD, NOSIP, NOTCH2, NOTCH4, NOTO, NPC1, NPIPB11, NPIPB12, NPIPB13,
NPIPB3, NPIPB4, NPIPB5, NPR1, NPR3, NR1D2, NR2C2, NR2C2AP, NR4A1, NRBP1,
NRIP1, NSD2, NSUN4, NT5C3A, NT5DC2, NTNG2, NTRK2, NUAK1, NUDCD3,
NUMA1, NUP188, NUPR1, NYNRIN, NYX, OAS1, OAS2, OAS3, OASL, OBSCN, ODC1,
OLIG1, OLR1, ONECUT1, OR13G1, OR2AT4, OR2C1, ORAI2, OS9, OSBPL10, OSGEP,
OTUD3, OTUD7B, OTULINL, OTX1, OXCT1, P2RY13, P2RY14, P3H1, P4HB, PACS2,
PACSIN2, PACSIN3, PADI2, PADI4, PAK1, PALD1, PALMD, PAMR1, PAN2, PAPPA,
PAQR9, PARD3, PARL, PARP2, PARP9, PARVB, PATL1, PAX9, PAXX, PBXIP1,
PCAT1, PCBP1, PCDH1, PCDH12, PCGF3, PCGF5, PCID2, PCNX3, PCSK9, PCTP,
PDCD11, PDE2A, PDE4B, PDE9A, PDGFB, PDGFRB, PDGFRL, PDIA5, PDIA6, PDK4,
PDLIM3, PDLIM4, PDPK1, PDRG1, PDXK, PEAK1, PELI1, PELO, PER1, PER3, PEX5,
PFKL, PFN1, PGAM4, PGAP6, PGF, PGLYRP1, PHC2, PHF11, PHLDA2, PHLDA3,
PHLDB1, PHPT1, PI4K2A, PI4K2B, PI4KA, PI4KB, PIAS3, PIEZO1, PIGR, PIGU,
PIH1D1, PIK3AP1, PIK3IP1, PIK3R4, PILRA, PIM2, PIN4, PIP4P1, PISD, PJA1, PKLR,
PKN1, PLA2G7, PLAAT2, PLAAT3, PLAC8, PLAC9, PLBD1, PLCD1, PLCG1, PLEK,
PLEKHA4, PLEKHG3, PLEKHJ1, PLEKHM1, PLEKHM2, PLEKHO1, PLEKHO2,
PLEKHS1, PLK2, PLLP, PLP2, PLPP1, PLPP3, PLSCR4, PLXNA2, PLXNB1, PLXNB2,
PLXND1, PML, PMPCB, PNISR, PNLDC1, PNMA5, PNP, PNRC1, PODN, PODXL,
POFUT2, POLDIP3, POLR1E, POLR2D, POLR2K, POLR3C, POLR3GL, POM121C,
POMT2, POMZP3, POP5, POU2AF1, POU6F1, PPARGC1B, PPDPF, PPIB, PPID, PPIG,
PPIH, PPM1F, PPM1M, PPPI1CA, PPP1R12A, PPP1R12C, PPP1R13B, PPP1R14A,
PPP1R14B, PPP1R16B, PPP1R3C, PPP2R5B, PPP3R1, PPP6R2, PPRC1, PQBP1, PRDM2,
PRDM8, PRDX1, PRDX2, PRELID1, PRKAB1, PRKAR1B, PRKAR2A, PRKX, PRMT1,
PRMT7, PRMT9, PROK2, PRPF39, PRPF6, PRR13, PRR14L, PRR3, PRR36, PRSS16,
PRX, PSENEN, PSMA4, PSMB1, PSMB10, PSMB4, PSMC1, PSMC3, PSMC5, PSMC6,
PSMD2, PSMD4, PSMD6, PSMD7, PSMD8, PSME3, PSTPIP2, PTCD3, PTDSS2, PTGDS,
PTGIS, PTH1R, PTK2B, PTK7, PTMS, PTP4A3, PTP A, PTPN1, PTPN13, PTPN3, PTPRC,
PTPRM, PTPRZ1, PUF60, PUS1, PVR, PWP2, PWWP2A, PWWP3A, PWWP3B, PXDC1,
PXDN, PXN, PYCR2, PYGO2, QARS1, QSOX1, QTRT1, R3HCC1, RAB11FIP3, RAB34,
RAB4A, RAB8B, RABGAP1L, RABGGTA, RAC2, RACK1, RAF1, RAI2, RALGDS,
RAMP3, RANBP10, RANBP3, RANGAP1, RAPGEF1, RAPGEF3, RARRES2, RASA3,
RASD2, RASGEF1B, RASGEF1C, RASIP1, RASL11A, RASL12, RASSF1, RASSF10,
RBFOX2, RBM10, RBM11, RBM19, RBM33, RBM6, RBP4, RCAN1, RCC2, RCE1, RCN2,
RCOR1, RECK, REPS2, RETREG3, REV3L, REX1BD, RFC1, RFLNB, RFTN1, RFXANK,
RGCC, RGL4, RGP1, RGPD1, RGPD2, RGPD3, RGPD4, RGPD5, RGPD6, RGS12, RGS18,
RGS3, RGS4, RHBDD2, RHBDF1, RHOC, RHOH, RIMS3, RIOK2, RIPOR2, RMDN1,
RMND5A, RMND5B, RNASE2, RND1, RND3, RNF113A, RNF122, RNF144A, RNF149,
RNF152, RNF168, RNF175, RNF185, RNF19B, RNF25, RNF4, RNF40, RNMT, RNPS1,
RNU4ATAC, ROBO3, ROBO4, RP2, RPAIN, RPH3A, RPIA, RPL13, RPL18, RPL3,
RPL36AL, RPL8, RPLP0, RPRD1A, RPS19BP1, RPS28, RPS5, RPS6KA2, RPS9, RPSA,
RPTOR, RRAD, RRP1, RRP12, RRP7A, RSAD1, RSAD2, RTKN2, RTN3, RTP4,
RUVBL2, RXRB, S100A11, S100A12, S100A13, S100A16, S100A2, S100A8, S100A9,
S100P, S100PBP, S1PR2, SAA1, SAA2, SAE1, SAFB, SAFB2, SAMD9, SAMD9L,
SAMSN1, SARS1, SASH1, SAT1, SCAF4, SCAMP4, SCGB1A1, SCN2A, SCN4B, SCN7A,
SCNN1G, SCP2D1, SCRN2, SCYL1, SDC3, SDHAF1, SEC16A, SEC23A, SEC23IP,
SELENBP1, SELL, SELP, SEM1, SEMA3B, SEMA3F, SEMA4C, SEMA6B, SENP3,
SENP5, SEPHS1, SEPTIN14, SEPTIN8, SEPTIN9, SERF2, SERPINA1, SERPINB2,
SERPINB6, SERPINB9, SERPINE2, SERTAD1, SERTAD3, SESN2, SEZ6L2, SF3A2,
SF3B1, SF3B4, SF3B5, SFN, SFRP2, SFT2D1, SFTPC, SGCA, SGIP1, SGK1, SGSM2,
SGTA, SH3BGRL3, SH3BP5, SH3D19, SH3PXD2A, SH3PXD2B, SH3RF1, SH3TC1,
SHANK3, SHC2, SHE, SHFL, SHOC1, SIDT2, SIGLEC14, SIGLEC5, SIK2, SIRPB1,
SIRT1, SIVA1, SKIV2L, SKOR1, SLAMF7, SLC24A4, SLC25A23, SLC25A25,
SLC25A29, SLC25A3, SLC25A30, SLC25A36, SLC25A4, SLC25A44, SLC25A6,
SLC26A8, SLC30A4, SLC35A4, SLC35B2, SLC35F1, SLC35F2, SLC35G2, SLC38A5,
SLC39A7, SLC39A8, SLC41A3, SLC44A2, SLC5A7, SLC66A2, SLC6A8, SLC9A1,
SLCO2B1, SLF2, SLIT2, SLIT3, SLK, SMAD7, SMAP2, SMARCA4, SMARCB1,
SMARCD2, SMC3, SMG5, SMIM25, SMIM27, SMIM3, SMPD4, SMTN, SMURF1,
SNAH, SNAPC2, SNCG, SNHG3, SNRNP200, SNRNP70, SNRPA1, SNX10, SNX11,
SNX25, SOCS1, SOCS2, SOGA1, SOS1, SP110, SP140, SP140L, SPAG6, SPARCL1,
SPATA16, SPATA31E1, SPEM2, SPG7, SPHK1, SPH, SPOUT1, SPRED1, SPRR2E,
SPRYD3, SPTLC2, SQSTM1, SREK1, SRGN, SRSF5, SRSF7, SSH1, SSNA1, ST6GAL1,
ST6GAL2, ST6GALNAC4, ST8SIA2, STAB1, STAG1, STAP1, STAP2, STARD3,
STARD8, STARD9, STAT1, STAT2, STAT4, STAT5A, STC1, STEAP1, STEAP4, STK16,
STK25, STK38, STK40, STN1, STRIP1, STRN4, STUB1, STX11, STXBP6, STYX,
SUCNR1, SUGP1, SULT1A1, SULT1A2, SULT1B1, SUN1, SUN3, SUN5, SUPT6H,
SURF1, SURF6, SYAP1, SYN1, SYNE1, SYNM, SYNPO, SYNPO2L, SYT11, TACC1,
TAF1, TAFA2, TAGAP, TAGLN, TANC1, TANC2, TAOK3, TAPI, TARBP2, TARS3,
TATDN2, TAZ, TBC1D17, TBC1D2B, TBC1D9, TBC1D9B, TBCEL, TBP, TBX2, TCEA3,
TCEAL9, TCERG1, TCF21, TCF24, TCF25, TCF7L1, TCN1, TEAD3, TECPR1, TEF,
TET2, TEX10, TEX264, TEX9, TFEC, TFIP11, TFPI2, TGFBR1, TGFBR2, THAP2,
THAP7, THBD, THBS3, THEM4, THOC6, THSD7B, THUMPD3, TIMM13, TIMM23,
TIMM44, TIMP3, TINAGL1, TJAP1, TLE3, TLE4, TLR1, TLR7, TM4SF20, TMBIM1,
TMC6, TMEM100, TMEM109, TMEM125, TMEM154, TMEM171, TMEM176B,
TMEM200B, TMEM208, TMEM235, TMEM245, TMEM252, TMEM255B, TMEM39B,
TMEM45B, TMEM51, TMEM52B, TMEM71, TMEM74B, TMEM88, TMOD1, TMPRSS2,
TMTC1, TNF, TNFAIP1, TNFAIP6, TNFRSF10B, TNFRSF14, TNFSF10, TNFSF13B,
TNFSF14, TNFSF8, TNIK, TNIP2, TNIP3, TNNC1, TNS1, TNS2, TNXB, TOE1, TOLLIP,
TOM1L2, TOMM34, TOP2A, TOR1B, TP53BP1, TP53BP2, TP73, TPCN1, TPD52L2,
TPP1, TPRG1L, TPSAB1, TPSB2, TPST1, TRAF2, TRAF5, TRAK1, TRAK2, TRAPPC10,
TREM1, TREML4, TRGC1, TRIB3, TRIM21, TRIM22, TRIM29, TRIM38, TRIM5,
TRIM69, TRIO, TRIP10, TRIR, TRIT1, TRMT61A, TRNAU1AP, TRPC4AP, TRPC6,
TRPM5, TRPM8, TRRAP, TSC1, TSC22D3, TSFM, TSPAN18, TSPAN4, TSPAN9,
TSPYL2, TSR1, TSSC4, TTC14, TTC31, TTC7A, TTL, TUBA1A, TUBA1B, TUBA1C,
TUBA4A, TUBB2A, TUBB2B, TUBB4B, TUBB6, TUBGCP2, TUBGCP3, TUBGCP6,
TULP3, TUSC7, TUT4, TWF2, TWNK, TXLNA, TXNDC11, TYROBP, UACA, UBA7,
UBB, UBC, UBE2J2, UBE2L6, UBIAD1, UBL4A, UBR4, UBTF, UBXN1, UCP2,
UNC13B, UNC80, UNC93A, UPK2, UPK3B, UPP1, UQCC2, UQCRC1, URB1, URGCP,
URM1, UROD, USB1, USP15, USP18, USP19, USP20, USP22, USP30, USP40, USP47,
USP53, USP7, UTP14C, UTP20, UTP6, VASN, VASP, VAX1, VCPKMT, VIPR1, VLDLR,
VNN2, VOPP1, VPS11, VPS13D, VPS16, VPS18, VPS28, VPS37B, VPS39, VPS8,
VSIG10L, VSIG2, VSTM4, VWA7, VWF, WAS, WASHC1, WBP1L, WDR13, WDR37,
WDR43, WDR74, WDR75, WDR91, WIPF1, WIPI1, WIPI2, WNT2B, XAB2, XAF1,
XAGE1A, XAGE1B, XKR9, XPC, XPO5, XRCC6, XRN1, YARS1, YKT6, YPEL3, ZBP1,
ZBTB10, ZBTB17, ZBTB34, ZBTB44, ZBTB5, ZC3H7B, ZC3HAV1, ZCCHC24, ZCCHC7,
ZDHHC11B, ZDHHC19, ZDHHC7, ZFAND2B, ZFP36L2, ZFYVE19, ZMYM2, ZNF106,
ZNF133, ZNF154, ZNF160, ZNF263, ZNF3, ZNF320, ZNF330, ZNF331, ZNF384,
ZNF385A, ZNF385D, ZNF394, ZNF395, ZNF426, ZNF451, ZNF480, ZNF496, ZNF513,
ZNF585B, ZNF599, ZNF622, ZNF692, ZNF697, ZNF761, ZNF83, ZNF841, ZNRD2,
ZNRF4, ZPLD1, ZRANB1, ZRSR2, ZSCAN18, ZSWIM7, ZSWIM8, ZZEF1
BAL-CoV2 to PBMC-CoV2 (8,959 DEGs in Airway)
A2M, A2ML1, A4GNT, AAAS, AACS, AADAT, AAGAB, AAMP, AANAT, AARS2,
AASDHPPT, AASS, AATF, AATK, ABCA1, ABCA12, ABCA13, ABCA2, ABCA3,
ABCA5, ABCA6, ABCA7, ABCB1, ABCB10, ABCB11, ABCB7, ABCB8, ABCB9,
ABCC1, ABCC10, ABCC2, ABCC3, ABCC4, ABCD1, ABCD2, ABCD3, ABCD4, ABCE1,
ABCF2, ABCF3, ABCG1, ABCG8, ABHD10, ABHD12, ABHD12B, ABHD13, ABHD16A,
ABHD17A, ABHD17B, ABHD17C, ABHD2, ABHD3, ABHD6, ABHD8, ABH, ABI3BP,
ABITRAM, ABL1, ABL2, ABT1, ABTB1, ACAA1, ACAA2, ACACB, ACAD10, ACAD8,
ACAD9, ACADL, ACADM, ACADS, ACADSB, ACADVL, ACAP1, ACAP2, ACAP3,
ACAT1, ACBD3, ACBD4, ACBD5, ACCS, ACD, ACE, ACE2, ACHE, ACIN1, ACLY,
ACO1, ACO2, ACOD1, ACOT1, ACOT11, ACOT13, ACOT2, ACOT4, ACOT7, ACOT8,
ACOX1, ACOX2, ACOX3, ACP2, ACP5, ACPP, ACRBP, ACSF3, ACSL4, ACSL5,
ACSL6, ACSM3, ACSM6, ACSS1, ACSS2, ACSS3, ACTB, ACTG1, ACTL6A, ACTN4,
ACTR1A, ACTR1B, ACTR2, ACTR3C, ACVR1, ACVR1C, ACVR2A, ACVRL1, ACYP2,
ADA2, ADAM10, ADAM15, ADAM19, ADAM22, ADAM28, ADAM32, ADAM8,
ADAMDEC1, ADAMTS10, ADAMTS19, ADAMTS2, ADAMTS9, ADAMTSL4, ADAP1,
ADARB1, ADCK1, ADCK2, ADCK5, ADCY2, ADCY3, ADCY6, ADCY7, ADCY9,
ADD1, ADD2, ADD3, ADGB, ADGRA2, ADGRE3, ADGRE5, ADGRF1, ADGRF5,
ADGRG1, ADGRG3, ADGRG5, ADGRG6, ADGRL1, ADGRL2, ADGRV1, ADH1B,
ADH1C, ADH5, ADH6, ADH7, ADHFE1, ADIPOR1, ADIPOR2, ADM, ADNP, ADNP2,
ADORA1, ADORA3, ADPGK, ADPRHL1, ADPRM, ADRM1, ADSL, ADSS1, ADTRP,
AEBP1, AEN, AFAP1L2, AFDN, AFF2, AFF3, AFF4, AFG1L, AFG3L2, AGAP1, AGAP2,
AGAP4, AGAP6, AGAP9, AGBL2, AGBL5, AGER, AGFG1, AGFG2, AGGF1, AGMAT,
AGO1, AGO2, AGO4, AGPAT1, AGPAT2, AGPAT3, AGPS, AGR2, AGR3, AGRN,
AGRP, AGTPBP1, AGTRAP, AHDC1, AHU, AHNAK, AHSA1, AHSP, AICDA, AIF1,
AIFM1, AIG1, AIM2, AIP, AJM1, AK1, AK4, AK6, AK7, AK9, AKAP1, AKAP11,
AKAP12, AKAP13, AKAP14, AKAP17A, AKAP3, AKAP5, AKAP6, AKAP7, AKAP8,
AKAP8L, AKAP9, AKIRIN1, AKIRIN2, AKNA, AKR1A1, AKR1B1, AKR1C1, AKR1C2,
AKR1C3, AKR1C4, AKR7A2, AKT1, AKT1S1, AKT2, AKT3, AKTIP, ALAD, ALAS1,
ALAS2, ALCAM, ALDH16A1, ALDH1A1, ALDH1A2, ALDH1A3, ALDH1B1, ALDH2,
ALDH3A1, ALDH3A2, ALDH3B1, ALDH3B2, ALDH4A1, ALDH6A1, ALDH7A1,
ALDOA, ALG1, ALG12, ALG14, ALG2, ALG6, ALG9, ALK, ALKBH1, ALKBH4,
ALKBH5, ALKBH6, ALMS1, ALOX12, ALOX12B, ALOX15, ALOX5AP, ALPK3,
ALYREF, AMDHD2, AMER1, AMFR, AMOTL1, AMOTL2, AMPD2, AMPD3, AMY1B,
AMY2A, AMY2B, AMZ2, ANAPC10, ANAPC11, ANAPC13, ANAPC15, ANAPC2,
ANAPC4, ANAPC5, ANGPT4, ANGPTL1, ANK1, ANK2, ANK3, ANKDD1A, ANKFN1,
ANKH, ANKHD1, ANKHD1-EIF4EBP3, ANKIB1, ANKLE2, ANKMY1, ANKMY2,
ANKRA2, ANKRD10, ANKRD12, ANKRD13A, ANKRD13D, ANKRD17, ANKRD18B,
ANKRD22, ANKRD26, ANKRD27, ANKRD28, ANKRD29, ANKRD34B, ANKRD35,
ANKRD36B, ANKRD39, ANKRD46, ANKRD50, ANKRD52, ANKRD54, ANKRD55,
ANKRD6, ANKRD65, ANKRD66, ANKRD9, ANKS1A, ANKUB1, ANKZF1, ANO10,
ANO5, ANO6, ANO8, ANO9, ANOS1, ANP32A, ANP32B, ANPEP, ANTXR1, ANTXR2,
ANXA1, ANXA11, ANXA2, ANXA3, ANXA4, ANXA5, ANXA6, AOAH, AOC2, AOC3,
AOPEP, AOX1, AP1AR, AP1B1, AP1G2, AP1M1, AP1S1, AP1S2, AP2A1, AP2M1,
AP3B1, AP3M1, AP3S2, AP4B1, AP4E1, AP5B1, AP5Z1, APAF1, APBA1, APBA2,
APBA3, APBB1, APBBHP, APBB2, APBB3, APC, APC2, APEH, APELA, APEX2,
APH1A, APIP, APLF, APLP2, APMAP, APOBEC3B, APOBEC3C, APOBEC3F,
APOBEC4, APOBR, APOC1, APOE, APOL2, APOL3, APOL4, APOM, APOO, APOOL,
APP, APPL2, APRT, APTX, AQP10, AQP4, AQP5, AQR, AR, ARAF, ARAP1, ARAP3,
ARCN1, AREG, ARF1, ARF3, ARF5, ARFGAP1, ARFGAP2, ARFGAP3, ARFGEF1,
ARFGEF3, ARFIP1, ARFRP1, ARGLU1, ARHGAP1, ARHGAP10, ARHGAP11A,
ARHGAP12, ARHGAP15, ARHGAP18, ARHGAP19, ARHGAP21, ARHGAP25,
ARHGAP27, ARHGAP30, ARHGAP32, ARHGAP33, ARHGAP4, ARHGAP42,
ARHGAP44, ARHGAP5, ARHGAP6, ARHGAP9, ARHGDIA, ARHGDIB, ARHGEF1,
ARHGEF16, ARHGEF18, ARHGEF19, ARHGEF2, ARHGEF28, ARHGEF3, ARHGEF35,
ARHGEF38, ARHGEF39, ARHGEF40, ARHGEF6, ARHGEF7, ARID1A, ARID1B,
ARID2, ARID3A, ARID3B, ARID4A, ARID5A, ARIH1, ARIH2, ARL1, ARL10, ARL13B,
ARL14, ARL14EP, ARL15, ARL16, ARL17A, ARL2BP, ARL3, ARL4A, ARL4C, ARL5A,
ARL6, ARL6IP6, ARL8A, ARL8B, ARMC2, ARMC3, ARMC4, ARMC5, ARMC6,
ARMC7, ARMC8, ARMC9, ARMCX1, ARMCX2, ARMCX4, ARMH1, ARMH3, ARMT1,
ARNTL, ARNTL2, ARPCIA, ARPC1B, ARPC2, ARPC3, ARPC4, ARPP19, ARRB2,
ARRDC1, ARRDC2, ARSA, ARSD, ARSG, ARV1, ARVCF, ASAP3, ASB1, ASB6, ASB8,
ASCC1, ASCC2, ASCC3, ASF1A, ASF1B, ASGR1, ASGR2, ASH1L, ASIC3, ASL,
ASMTL, ASPH, ASPHD2, ASPN, ASPSCR1, ASRGL1, ASS1, ASTN2, ASXL1, ASXL2,
ASXL3, ATAD2, ATAD2B, ATAD3A, ATAD3B, ATAD3C, ATF1, ATF2, ATF3, ATF4,
ATF6, ATF6B, ATF7IP, ATG101, ATG12, ATG13, ATG14, ATG16L2, ATG2A, ATG2B,
ATG3, ATG4A, ATG4B, ATG4C, ATG4D, ATG5, ATG7, ATG9A, ATL2, ATL3, ATM,
ATMIN, ATOX1, ATP10A, ATP10B, ATP11A, ATP12A, ATP13A1, ATP13A2, ATP13A3,
ATP1A1, ATP1B1, ATP1B4, ATP23, ATP2A2, ATP2A3, ATP2B1, ATP2B2, ATP2B4,
ATP2C2, ATP5F1A, ATP5F1B, ATP5F1C, ATP5F1D, ATP5F1E, ATP5IF1, ATP5MC1,
ATP5MC2, ATP5MC3, ATP5MD, ATP5ME, ATP5MF, ATP5MG, ATP5MPL, ATP5PB,
ATP5PD, ATP5PF, ATP5PO, ATP6AP1, ATP6AP1L, ATP6AP2, ATP6V0A2, ATP6V0A4,
ATP6V0B, ATP6V0C, ATP6V0D1, ATP6V0D2, ATP6V0E1, ATP6V0E2, ATP6V1A,
ATP6V1C1, ATP6V1D, ATP6V1E1, ATP6V1F, ATP6V1G1, ATP6V1H, ATP7A, ATP7B,
ATP8A1, ATP8B1, ATP8B2, ATP8B3, ATP9A, ATPAF1, ATPAF2, ATXN1L, ATXN2,
ATXN2L, ATXN3, ATXN7, ATXN7L1, ATXN7L3, ATXN7L3B, AUH, AUP1, AURKA,
AURKB, AUTS2, AVEN, AVPI1, AXIN1, AXL, AZGP1, AZI2, AZIN2, AZU1, B2M,
B3GALNT1, B3GALNT2, B3GALT5, B3GALT6, B3GAT1, B3GAT3, B3GNT2, B3GNT3,
B3GNT5, B3GNT7, B3GNT8, B3GNTL1, B4GALT2, B4GALT3, B4GALT4, BACE2,
BACH2, BAD, BAG2, BAG3, BAG6, BAHCC1, BAHD1, BAIAP2L1, BAK1, BANF1,
BANK1, BANP, BAP1, BATF3, BAX, BAZ1A, BAZ1B, BAZ2A, BAZ2B, BBC3, BBIP1,
BBS10, BBS12, BBS2, BBS9, BBX, BCAM, BCAP29, BCAP31, BCAR3, BCAS1, BCAS2,
BCAT1, BCCIP, BCKDHA, BCKDHB, BCKDK, BCL11B, BCL2A1, BCL2L1, BCL2L11,
BCL2L14, BCL2L15, BCL3, BCL6, BCL9L, BCLAF1, BCLAF3, BCOR, BCORL1, BCR,
BDH2, BDKRB2, BDP1, BECN1, BEND2, BEND4, BEND5, BEND7, BEST3, BET1,
BET1L, BEX1, BFSP1, BHLHA15, BHLHE40, BHLHE41, BICD1, BICD2, BICDL2,
BICRA, BICRAL, BIN1, BIN2, BIN3, BIRC3, BIRC5, BLK, BLNK, BLOC1S1, BLOC1S2,
BLOC1S4, BLOC1S5, BLOC1S6, BLVRA, BLZF1, BMF, BMP2K, BMP3, BMP6,
BMPR1A, BMPR1B, BMPR2, BMS1, BMX, BNIP1, BNIP3, BNIP3L, BOD1, BOLA1,
BOLA2B, BOLA3, BOP1, BORCS5, BORCS6, BORCS7, BORCS8, BPGM, BPHL,
BPIFA1, BPIFB1, BPIFB4, BPTF, BRAT1, BRCA1, BRCA2, BRCC3, BRD1, BRD2,
BRD8, BRD9, BRF1, BRF2, BRI3, BRI3BP, BRMS1, BRPF1, BRPF3, BRWD1, BSCL2,
BSDC1, BSG, BSPRY, BST1, BST2, BTAF1, BTBD1, BTBD10, BTBD11, BTBD19,
BTBD2, BTBD3, BTBD7, BTBD8, BTF3, BTF3L4, BTG1, BTG2, BTK, BTLA, BTN2A1,
BTN3A1, BTN3A2, BTNL9, BTRC, BUD13, BUD23, BZW1, BZW2, C10orf143, C10orf25,
C10orf67, C11orf1, C11orf16, C11orf21, C11orf24, C11orf45, C11orf65, C11orf68,
C11orf71, C11orf74, C11orf88, C11or97, C12orf10, C12orf4, C12orf43, C12orf45,
C12orf49, C12orf60, C12orf73, C12orf74, C13orf46, C14orf119, C15orf39, C15orf40,
C15orf48, C15orf62, C15orf65, C16orf54, C16orf58, C16orf70, C16orf72, C16orf74,
C16orf87, C16orf89, C16orf91, C17orf100, C17orf49, C17orf58, C17orf80, C17orf97,
C18orf21, C18orf25, C18orf32, C18orf54, C19orf25, C19orf38, C19orf47, C19orf53,
C19orf54, C19orf71, C1D, C1GALT1, C1GALT1C1, C1orf115, C1orf116, C1orf122,
C1orf131, C1orf141, C1orf158, C1orf159, C1orf162, C1orf189, C1orf194, C1orf198,
C1orf21, C1orf216, C1orf50, C1orf53, C1orf54, C1orf87, C1QA, C1QB, C1QC, C1QTNF6,
C1RL, C2, C20orf194, C20orf85, C20or96, C21or91, C2CD2L, C2orf15, C2orf42, C2orf49,
C2orf68, C2orf73, C2orf76, C2orf88, C2or92, C3, C3AR1, C3orf14, C3orf18, C3orf49,
C3orf62, C3orf67, C3orf84, C4A, C4B, C4orf19, C4orf3, C4orf46, C5AR1, C5AR2,
C5orf22, C5orf24, C5orf30, C5orf49, C5orf51, C5orf58, C6, C6orf120, C6orf132, C6orf136,
C6orf163, C6orf226, C6orf47, C6orf62, C6orf89, C7orf26, C7orf50, C7orf57, C8B, C8orf34,
C8orf37, C9orf116, C9orf129, C9orf135, C9orf139, C9orf152, C9orf16, C9orf24, C9orf64,
C9orf78, CA1, CA13, CA14, CA2, CA4, CA5B, CA8, CAAP1, CABCOCO1, CABIN1,
CABLES1, CABLES2, CABP4, CACHD1, CACNA1E, CACNA1F, CACNA1I,
CACNA2D1, CACNA2D2, CACNA2D3, CACNA2D4, CACNB1, CACTIN, CACUL1,
CACYBP, CAD, CADPS2, CALCOCO2, CALCR, CALHM2, CALHM5, CALHM6,
CALM1, CALM2, CALM3, CALU, CAMK1, CAMK1D, CAMK2D, CAMK2G,
CAMK2N1, CAMK4, CAMKK1, CAMKK2, CAMLG, CAMP, CAMSAP2, CAMTA1,
CAMTA2, CANT1, CAP1, CAP2, CAPN1, CAPN10, CAPN12, CAPN13, CAPN15,
CAPN2, CAPN3, CAPN5, CAPN7, CAPNS1, CAPRIN1, CAPS2, CAPSL, CAPZAI,
CAPZB, CARD11, CARD19, CARD6, CARD8, CARD9, CARF, CARHSP1, CARM1,
CARMIL1, CARMIL2, CARMIL3, CARNMT1, CARNS1, CASC1, CASC3, CASC4,
CASK, CASKIN2, CASP3, CASP4, CASP5, CASP7, CASP8, CASP8AP2, CASS4,
CASTOR2, CASZ1, CATSPER1, CATSPERB, CATSPERD, CAV3, CAVIN1, CAVIN2,
CAVIN4, CBFA2T2, CBFA2T3, CBL, CBLL1, CBR1, CBS, CBSL, CBWD6, CBX1, CBX3,
CBX4, CBX6, CBX7, CBY1, CC2D1A, CC2D1B, CC2D2A, CCAR1, CCAR2, CCDC102B,
CCDC106, CCDC107, CCDC110, CCDC112, CCDC113, CCDC117, CCDC124, CCDC125,
CCDC127, CCDC130, CCDC134, CCDC137, CCDC138, CCDCM1, CCDCM6, CCDC15,
CCDC158, CCDC159, CCDC160, CCDC167, CCDC17, CCDC170, CCDC171, CCDC178,
CCDC18, CCDC180, CCDC181, CCDC186, CCDC187, CCDC190, CCDC200, CCDC22,
CCDC25, CCDC28B, CCDC30, CCDC32, CCDC33, CCDC34, CCDC36, CCDC40,
CCDC51, CCDC57, CCDC58, CCDC6, CCDC60, CCDC61, CCDC65, CCDC66, CCDC69,
CCDC7, CCDC71, CCDC71L, CCDC74A, CCDC74B, CCDC78, CCDC80, CCDC81,
CCDC85B, CCDC85C, CCDC88A, CCDC88B, CCDC88C, CCDC89, CCDC9, CCDC90B,
CCDC91, CCDC92, CCDC97, CCDC9B, CCHCR1, CCL18, CCL2, CCL20, CCL23, CCL3,
CCL3L1, CCL4, CCL4L2, CCL5, CCL7, CCL8, CCM2, CCN1, CCN2, CCNB2, CCNB3,
CCND1, CCND2, CCND3, CCNDBP1, CCNE2, CCNF, CCNG2, CCNI, CCNJL, CCNK,
CCNL1, CCNL2, CCNO, CCNT1, CCNY, CCNYL1, CCP110, CCR1, CCR4, CCR5, CCR7,
CCRL2, CCS, CCSAP, CCSER2, CCT7, CCZ1B, CD101, CD14, CD160, CD164, CD180,
CD19, CD1A, CD1C, CD1D, CD2, CD200, CD22, CD226, CD24, CD244, CD247, CD27,
CD274, CD276, CD28, CD2AP, CD2BP2, CD300A, CD300C, CD300H, CD300LB, CD33,
CD36, CD37, CD3D, CD3E, CD3EAP, CD3G, CD4, CD40LG, CD44, CD46, CD47, CD48,
CD5, CD52, CD53, CD59, CD5L, CD6, CD68, CD69, CD7, CD72, CD79A, CD79B, CD80,
CD81, CD82, CD84, CD8A, CD8B, CD8B2, CD9, CD93, CD96, CD99, CD99L2, CDA,
CDADC1, CDAN1, CDC123, CDC14A, CDC14B, CDC20, CDC20B, CDC25A, CDC25B,
CDC26, CDC27, CDC34, CDC37, CDC37L1, CDC42BPA, CDC42BPB, CDC42EP1,
CDC42EP2, CDC42EP3, CDC42EP4, CDC42SE1, CDC42SE2, CDC5L, CDC6, CDC7,
CDC73, CDCA3, CDCA4, CDCA5, CDCA7L, CDCA8, CDCP1, CDH13, CDH23, CDH26,
CDHR3, CDIP1, CDIPT, CDK10, CDK11A, CDK11B, CDK13, CDK14, CDK16, CDK2,
CDK20, CDK2AP1, CDK4, CDK5R1, CDK5RAP2, CDK5RAP3, CDK6, CDK7, CDK9,
CDKAL1, CDKL5, CDKN1A, CDKN1C, CDKN2AIP, CDKN2B-AS1, CDKN2D, CDPF1,
CDR2, CDS1, CDS2, CDV3, CDYL, CDYL2, CEACAM1, CEACAM19, CEACAM3,
CEACAM4, CEACAM5, CEACAM6, CEACAM7, CEBPA, CEBPB, CEBPZ, CELF1,
CELF2, CELSR1, CELSR2, CELSR3, CEMIP, CEMIP2, CENPB, CENPBD1, CENPH,
CENPK, CENPL, CENPO, CENPP, CENPQ, CENPS, CENPW, CEP112, CEP126, CEP128,
CEP162, CEP170, CEP19, CEP192, CEP250, CEP290, CEP295NL, CEP350, CEP68,
CEP70, CEP76, CEP78, CEP83, CEP85, CEP85L, CEP89, CEP95, CERK, CERKL, CERS2,
CERS4, CERS5, CERS6, CERT1, CES2, CES3, CES4A, CETN2, CETN3, CFAP20,
CFAP221, CFAP298, CFAP300, CFAP36, CFAP410, CFAP43, CFAP44, CFAP45, CFAP47,
CFAP52, CFAP53, CFAP54, CFAP57, CFAP61, CFAP65, CFAP70, CFAP97D2, CFDP1,
CFH, CFL1, CFL2, CFLAR, CFP, CFTR, CGAS, CGGBP1, CGN, CGNL1, CHAF1A,
CHCHD1, CHCHD10, CHCHD3, CHCHD6, CHCHD7, CHD1, CHD2, CHD3, CHD4,
CHD6, CHD8, CHD9, CHDH, CHEK1, CHEK2, CHERP, CHFR, CHIC1, CHIC2, CHID1,
CHKB, CHL1, CHM, CHMP1A, CHMPIB, CHMP2A, CHMP2B, CHMP4A, CHMP4B,
CHMP4C, CHMP5, CHMP6, CHMP7, CHN2, CHP1, CHP2, CHPF, CHPF2, CHPT1,
CHRDL2, CHRFAM7A, CHRNA2, CHRNA3, CHRNA7, CHST10, CHST11, CHST12,
CHST13, CHST15, CHST2, CHST4, CHST6, CHST9, CHSY1, CHTF18, CHTF8, CHTOP,
CHURC1, CIAO1, CIAO2A, CIAO3, CIAPIN1, CIC, CIITA, CIPC, CIR1, CIRBP, CISD1,
CITED4, CIZ1, CKAP2, CKAP2L, CKAP4, CKAP5, CKB, CKLF, CKMT1A, CKMT1B,
CKMT2, CKS2, CLASP1, CLASRP, CLC, CLCA2, CLCA4, CLCF1, CLCN2, CLCN3,
CLCN5, CLCN6, CLCN7, CLDN1, CLDN10, CLDN12, CLDN15, CLDN16, CLDN18,
CLDN23, CLDN3, CLDN4, CLDN5, CLDN9, CLDND1, CLDND2, CLEC11A, CLEC12A,
CLEC12B, CLEC17A, CLEC18A, CLEC1B, CLEC2D, CLEC4A, CLEC4D, CLEC7A,
CLGN, CLIC2, CLIC3, CLIC4, CLIC5, CLIC6, CLIP1, CLIP2, CLIP4, CLK1, CLK2, CLK3,
CLN3, CLN5, CLN6, CLOCK, CLP1, CLPP, CLPTM1, CLPTM1L, CLSTN1, CLSTN2,
CLSTN3, CLTA, CLTC, CLTCL1, CLU, CLUH, CLYBL, CMC1, CMC2, CMIP, CMPK1,
CMSS1, CMTM2, CMTM3, CMTM4, CMTM5, CMTM6, CMTR1, CMTR2, CMYA5,
CNBP, CNFN, CNGA4, CNIH1, CNIH4, CNKSR2, CNKSR3, CNN2, CNN3, CNNM3,
CNNM4, CNOT1, CNOT10, CNOT11, CNOT2, CNOT4, CNOT8, CNP, CNPPD1, CNPY3,
CNPY4, CNR2, CNRIP1, CNTD1, CNTLN, CNTN1, CNTN5, CNTNAP1, CNTNAP2,
CNTROB, COA3, COA4, COA6, COA7, COA8, COASY, COBL, COBLL1, COCH, COG1,
COG2, COG4, COG5, COG6, COL11A2, COL15A1, COL17A1, COL18A1, COL19A1,
COL1A1, COL26A1, COL4A4, COL5A3, COL6A2, COL8A1, COL9A2, COL9A3,
COLCA1, COLEC12, COLGALT1, COLGALT2, COLQ, COMMD1, COMMD10,
COMMD2, COMMD6, COMMD9, COMTD1, COP1, COPA, COPB2, COPE, COPG2,
COPRS, COPS3, COPS5, COPS7B, COPS9, COPZ1, COQ2, COQ6, COQ7, COQ8A,
COQ8B, CORO1A, CORO1B, CORO1C, CORO7, COTL1, COX14, COX17, COX19,
COX20, COX5A, COX6A1, COX6B1, COX6C, COX7A1, COX7B, COX7C, COX8A, CP,
CPAMD8, CPD, CPE, CPED1, CPLANE1, CPM, CPNE1, CPNE2, CPNE5, CPOX,
CPPED1, CPQ, CPSF1, CPSF2, CPSF3, CPSF4, CPSF6, CPT1A, CPT1B, CPTP, CPVL,
CR1, CR1L, CR2, CRACR2A, CRADD, CRAMP1, CRAT, CRCP, CRCT1, CREB3,
CREB3L1, CREB3L4, CREB5, CREBBP, CREBL2, CREBZF, CREG1, CRELD1, CREM,
CRHBP, CRIM1, CRIP1, CRIPT, CRISP3, CRISPLD2, CRK, CRMP1, CRNDE, CRNKL1,
CRNN, CROCC, CRTAC1, CRTAM, CRTAP, CRTC1, CRTC2, CRY2, CRYBB1,
CRYBG1, CRYBG2, CRYBG3, CRYL1, CRYM, CRYZ, CS, CSAD, CSE1L, CSF2RA,
CSF2RB, CSF3R, CSGALNACT1, CSGALNACT2, CSK, CSMD1, CSNK1A1, CSNK1D,
CSNK1E, CSNK1G1, CSNK1G2, CSNK1G3, CSPP1, CSRNP1, CSRNP2, CSRNP3,
CSRP2, CST3, CSTA, CSTB, CSTF1, CSTF3, CT83, CTBP1, CTBP1-AS, CTBS, CTC1,
CTDP1, CTDSP1, CTDSP2, CTIF, CTLA4, CTNNA1, CTNNAL1, CTNNB1, CTNNBIP1,
CTNND1, CTPS1, CTPS2, CTR9, CTSA, CTSC, CTSD, CTSF, CTSK, CTSL, CTSS,
CTSW, CTSZ, CTTNBP2, CTTNBP2NL, CTU2, CUBN, CUEDC1, CUEDC2, CUL2,
CUL3, CUL5, CUL7, CUL9, CUTC, CWC15, CWC22, CWC25, CWC27, CWF19L1,
CWF19L2, CX3CR1, CXADR, CXCL1, CXCL10, CXCL11, CXCL12, CXCL16, CXCL17,
CXCL2, CXCL3, CXCL6, CXCL9, CXCR1, CXCR3, CXCR4, CXorf38, CXorf40A,
CXorf40B, CXorf56, CXXC1, CXXC4, CXXC5, CYB561A3, CYB561D1, CYB561D2,
CYB5A, CYB5D2, CYB5R1, CYB5R2, CYB5R3, CYB5R4, CYB5RL, CYBA, CYBB,
CYBC1, CYBRD1, CYC1, CYFIP2, CYHR1, CYLD, CYP20A1, CYP27A1, CYP2B6,
CYP2E1, CYP2F1, CYP2J2, CYP2R1, CYP2S1, CYP39A1, CYP3A4, CYP3A5, CYP4B1,
CYP4F3, CYP4V2, CYP4X1, CYSLTR2, CYSRT1, CYSTM1, CYTH1, CYTH2, CYTH4,
CYTIP, CYYR1, CZIB, D2HGDH, DAAM1, DAAM2, DAB1, DAB2, DAB2IP, DAG1,
DAGLB, DALRD3, DAPK1, DAPK2, DAPK3, DARS2, DAZAP1, DAZAP2, DBF4B, DBI,
DBN1, DBNDD1, DBNL, DBP, DBT, DCAF11, DCAF12, DCAF13, DCAF15, DCAF16,
DCAF8, DCAKD, DCBLD1, DCBLD2, DCDC1, DCDC2, DCHS1, DCLK2, DCLRE1B,
DCLRE1C, DCP1B, DCSTAMP, DCTN1, DCTN2, DCTN3, DCTN4, DCTN6, DCTPP1,
DCUN1D3, DCUN1D4, DCUN1D5, DCXR, DDA1, DDAH1, DDB1, DDB2, DDC,
DDHD1, DDI2, DDIAS, DDIT4, DDO, DDOST, DDR1, DDR2, DDRGK1, DDX1, DDX10,
DDX11, DDX17, DDX18, DDX19A, DDX19B, DDX21, DDX23, DDX24, DDX27, DDX31,
DDX39A, DDX39B, DDX3X, DDX3Y, DDX41, DDX42, DDX47, DDX49, DDX5, DDX51,
DDX52, DDX54, DDX55, DDX56, DDX58, DDX59, DDX60, DEAF1, DEF6, DEF8,
DEFA1, DEFA1B, DEFA3, DEFB1, DELE1, DENND11, DENND1A, DENND1B,
DENND1C, DENND2B, DENND3, DENND4A, DENND4B, DENND4C, DENND5A,
DENND6A, DENND6B, DEPDC5, DEPP1, DEPTOR, DERL1, DERL2, DERL3, DESI2,
DET1, DEUP1, DEXI, DFFA, DFFB, DGAT1, DGAT2, DGCR2, DGCR6, DGCR8, DGKA,
DGKB, DGKD, DGKQ, DGKZ, DGLUCY, DGUOK, DHCR7, DHDH, DHFR, DHODH,
DHPS, DHRS1, DHRS12, DHRS13, DHRS3, DHRS4, DHRS7, DHRS9, DHRSX, DHTKD1,
DHX15, DHX16, DHX30, DHX32, DHX34, DHX37, DHX38, DHX40, DHX57, DHX9,
DIABLO, DIAPH1, DICER1, DIDO1, DIO2, DIP2A, DIP2C, DIPK1A, DIPK1B, DIPK2A,
DIS3, DIS3L2, DISP1, DIXDC1, DKC1, DKK1, DLC1, DLD, DLEC1, DLG3, DLG5,
DLST, DMAC1, DMAP1, DMBT1, DMD, DMKN, DMPK, DMRT3, DMRTA1, DMTN,
DMWD, DMXL1, DMXL2, DNAAF1, DNAH1, DNAH10, DNAH11, DNAH12, DNAH14,
DNAH17, DNAH2, DNAH3, DNAH5, DNAH6, DNAH7, DNAH8, DNAH9, DNAH,
DNAJA1, DNAJA3, DNAJB11, DNAJB12, DNAJB13, DNAJB14, DNAJB2, DNAJB4,
DNAJB6, DNAJB9, DNAJC10, DNAJC14, DNAJC15, DNAJC16, DNAJC17, DNAJC18,
DNAJC19, DNAJC21, DNAJC5, DNAJC5B, DNAJC7, DNAJC8, DNAJC9, DNAL1,
DNALI1, DNASE1, DNASE1L1, DNASE2B, DNHD1, DNM1, DNM1L, DNM2, DNM3,
DNMBP, DNPEP, DNPH1, DNTTIP1, DNTTIP2, DOC2A, DOCK1, DOCK10, DOCK2,
DOCK4, DOCK5, DOCK7, DOHH, DOK2, DOK3, DOK6, DOLK, DOT1L, DPEP2, DPF2,
DPF3, DPH1, DPH2, DPH3, DPH5, DPH7, DPM1, DPM2, DPM3, DPP3, DPP7, DPP9,
DPT, DPY19L3, DPY30, DPYSL2, DPYSL3, DR1, DRAM1, DRAP1, DRC1, DRG2,
DROSHA, DSC2, DSC3, DSE, DSG2, DSN1, DSP, DST, DSTN, DSTYK, DTNA, DTNB,
DTWD1, DTWD2, DTX1, DTX3, DTX3L, DTX4, DUOX1, DUOX2, DUOXA1, DUOXA2,
DUS1L, DUS2, DUS3L, DUSP1, DUSP11, DUSP14, DUSP2, DUSP22, DUSP23, DUSP28,
DUSP3, DUSP6, DUSP7, DUSP8, DUXAP9, DVL1, DVL2, DVL3, DXO, DYM,
DYNC1H1, DYNC1I2, DYNC1LI2, DYNC2H1, DYNC2LI1, DYNLL1, DYNLL2,
DYNLRB1, DYNLRB2, DYNLT1, DYNLT3, DYRK1B, DYRK4, DYSF, DZANK1,
DZIP1L, DZIP3, E2F1, E2F2, E2F3, E2F4, E2F7, E4F1, EAF1, EAPP, EARS2, EBAG9,
EBF1, EBLN2, EBNA1BP2, EBP, EBPL, ECE1, ECH1, ECHDC2, ECHS1, ECM1, ECPAS,
ECRG4, ECSIT, ECT2, ECT2L, EDAR, EDC3, EDC4, EDEM1, EDEM2, EDEM3, EDN1,
EDN2, EDNRA, EEA1, EEF1AKMT1, EEF1AKMT3, EEF1B2, EEFID, EEF2, EFCAB1,
EFCAB10, EFCAB11, EFCAB14, EFCAB6, EFCAB7, EFEMP1, EFHB, EFHD1, EFHD2,
EFL1, EFNA1, EFR3A, EFTUD2, EGF, EGFL6, EGFL7, EGFR, EGLN1, EGLN2, EGOT,
EGR1, EGR2, EGR3, EHBP1, EHBP1L1, EHD1, EHD2, EHD3, EHD4, EHF, EHHADH,
EHMT1, EHMT2, EID1, EIF1, EIF1AX, EIF1B, EIF2AK1, EIF2AK3, EIF2B2, EIF2B3,
EIF2B4, EIF2S1, EIF2S2, EIF2S3, EIF2S3B, EIF3B, EIF3C, EIF3CL, EIF3D, EIF3G, EIF3I,
EIF3L, EIF3M, EIF4A1, EIF4B, EIF4E, EIF4E3, EIF4EBP1, EIF4G1, EIF4G2, EIF4H,
EIF5A, EIF5AL1, EIF5B, EIF6, EIPR1, ELAC1, ELAC2, ELF2, ELF3, ELF4, ELF5, ELK1,
ELK4, ELL, ELL2, ELMO1, ELMO2, ELMO3, ELMOD1, ELMOD2, ELMOD3,
ELMSAN1, ELOA, ELOC, ELOVL1, ELOVL5, ELP3, ELP4, ELP5, ELP6, EMB, EMC1,
EMC10, EMC2, EMC8, EMC9, EMD, EME2, EMG1, EMID1, EMILIN2, EML1, EML2,
EML3, EML4, EML6, EMP2, EMP3, EMSY, ENAH, ENC1, ENDOD1, ENGASE, ENKUR,
ENO1, ENO2, ENO3, ENOPH1, ENPP2, ENPP3, ENPP5, ENSA, ENTPD1, ENTPD3,
ENTPD4, ENTPD6, EOGT, EP400, EPAS1, EPB41, EPB41L1, EPB41L2, EPB41L3,
EPB41L4A, EPB41L4B, EPB41L5, EPB42, EPC2, EPCAM, EPHA1, EPHA2, EPHB6,
EPHX2, EPM2AIP1, EPN1, EPN3, EPOR, EPPIN, EPPK1, EPS15, EPS15L1, EPS8, EQTN,
ERAL1, ERAP1, ERBB2, ERBB3, ERBB4, ERBIN, ERC1, ERCC1, ERCC2, ERCC3,
ERCC5, ERCC6L2, ERCC8, EREG, ERF, ERFE, ERFL, ERG, ERG28, ERGIC1, ERGIC2,
ERH, ERI1, ERI2, ERICH1, ERICH3, ERICH5, ERMAP, ERMN, ERN1, ERO1A, ERP29,
ERRFI1, ERV3-1, ESCO1, ESD, ESF1, ESPN, ESRP1, ESRP2, ESRRA, ESRRG, ESS2,
ESYT1, ESYT2, ETFDH, ETFRF1, ETNK1, ETS1, ETV2, ETV3, ETV5, EVA1B, EVI2A,
EVI5, EVI5L, EVL, EWSR1, EXD3, EXOC1, EXOC3, EXOC5, EXOC6B, EXOC7, EXOG,
EXOSC1, EXOSC10, EXOSC2, EXOSC4, EXOSC5, EXPH5, EXT2, EXTL3, EYA1, EYA2,
EYA3, EYA4, EYS, EZH1, EZH2, EZR, F11R, F12, F13A1, F2R, F2RL1, F2RL2, F2RL3,
F3, F5, F8A2, FA2H, FAAH, FAAP100, FAAP20, FABP3, FABP4, FABP5, FABP6,
FADS1, FADS2, FADS3, FAF1, FAF2, FAH, FAHD2A, FAHD2B, FAIM, FAM102A,
FAM102B, FAM104B, FAM106A, FAM107A, FAM107B, FAM110A, FAM110C,
FAM114A1, FAM117A, FAM117B, FAM118A, FAM118B, FAM120A, FAM120AOS,
FAM120B, FAM120C, FAM122B, FAM122C, FAM135A, FAM13A, FAM153A,
FAM153B, FAM156A, FAM156B, FAM160A1, FAM160A2, FAM160B1, FAM160B2,
FAM162A, FAM166B, FAM166C, FAM168A, FAM168B, FAM169A, FAM174A,
FAM174C, FAM177A1, FAM183A, FAM184A, FAM185A, FAM186B, FAM189B,
FAM193B, FAM199X, FAM200A, FAM204A, FAM209A, FAM20B, FAM210A,
FAM210B, FAM214A, FAM214B, FAM216B, FAM217B, FAM219A, FAM219B,
FAM220A, FAM222A, FAM222B, FAM229A, FAM229B, FAM234A, FAM3A, FAM3B,
FAM3D, FAM47E, FAM49A, FAM50A, FAM53B, FAM53C, FAM71F2, FAM72A,
FAM72B, FAM72C, FAM72D, FAM78A, FAM81B, FAM83B, FAM83D, FAM83G,
FAM89A, FAM8A1, FAM92A, FAM92B, FAM98C, FANCA, FANCC, FANCD2, FANCI,
FANK1, FAP, FAR1, FARP1, FARP2, FASN, FASTK, FASTKD3, FASTKD5, FAT1,
FAT3, FAU, FAXDC2, FBH1, FBLN2, FBLN5, FBN2, FBP1, FBRS, FBRSL1, FBXL12,
FBXL13, FBXL16, FBXL19, FBXL2, FBXL20, FBXL22, FBXL5, FBXL6, FBXL7, FBXL8,
FBX025, FBX028, FBX03, FBXO30, FBXO31, FBXO33, FBXO34, FBXO36, FBXO41,
FBXO42, FBXO46, FBXO48, FBXO6, FBXO7, FBXO8, FBXO9, FBXW10, FBXW2,
FBXW4, FBXW5, FBXW7, FBXW8, FCAR, FCER1A, FCERIG, FCER2, FCF1, FCGBP,
FCGR1A, FCGR1B, FCGR2A, FCGR3A, FCGRT, FCHO1, FCHO2, FCHSD1, FCHSD2,
FCMR, FCN1, FCN2, FCRL1, FCRL2, FCRL3, FCRL6, FCRLA, FCSK, FDCSP, FDPS,
FDX1, FECH, FEM1A, FEN1, FER, FER1L6, FERMT1, FERMT2, FERMT3, FES, FFAR4,
FGD2, FGD3, FGD4, FGD5, FGD6, FGF14, FGF20, FGFBP2, FGFR1, FGFR1OP2, FGFR2,
FGFR3, FGGY, FGL2, FGR, FHAD1, FHL1, FHL3, FHOD1, FICD, FILIP1, FILIPIL, FIS1,
FIZ1, FKBP11, FKBP15, FKBP1A, FKBP2, FKBP8, FKBPL, FKRP, FLACC1, FLCN, FLG,
FLG2, FLU, FLII, FLNA, FLOT2, FLRT3, FLT1, FLT3, FLT3LG, FLT4, FLVCR2,
FLYWCH1, FMNL1, FMNL2, FMO2, FMO3, FMO4, FMO5, FMOD, FMR1, FN1,
FNBP1L, FNBP4, FNDC10, FNDC3A, FNDC3B, FNIP1, FNIP2, FOCAD, FOLH1, FOLR1,
FOLR3, FOPNL, FOS, FOSB, FOSL1, FOSL2, FOXA1, FOXA3, FOXD2-AS1, FOXI2,
FOXH, FOXJ2, FOXJ3, FOXK1, FOXK2, FOXM1, FOXN1, FOXN3, FOXO3, FOXO4,
FOXP2, FOXP3, FOXQ1, FOXRED1, FPGS, FPGT, FPR1, FPR3, FRA10AC1, FRAT1,
FRAT2, FREM2, FRG1, FRK, FRMD4A, FRMD4B, FRMD5, FRMD6, FRMD8, FRMPD3,
FRRS1, FRS2, FRY, FRYL, FSCN1, FSD1, FSTL3, FTCDNL1, FTL, FTO, FTSJ1, FUBP1,
FUCA1, FUCA2, FUNDC1, FUOM, FURIN, FUS, FUT10, FUT11, FUT2, FUT3, FUT4,
FUT7, FUZ, FXR2, FXYD3, FXYD5, FYB2, FYCO1, FYN, FZD1, FZD5, FZD6, FZD7,
FZR1, GOS2, G6PC3, G6PD, GAA, GAB2, GABARAP, GABARAPL1, GABBR1, GABPA,
GABPB2, GABRB2, GABRP, GABRR2, GADD45B, GADD45G, GAK, GALE, GALK1,
GALNS, GALNT1, GALNT10, GALNT12, GALNT2, GALNT5, GALT, GANAB, GAPDH,
GAREM1, GAREM2, GARNL3, GART, GAS2L1, GAS2L3, GAS6, GATA1, GATA2,
GATA3, GATAD1, GATAD2A, GATD1, GATD3A, GATD3B, GATM, GBA2, GBE1,
GBF1, GBGT1, GBP1, GBP3, GBP6, GCA, GCC1, GCC2, GCDH, GCFC2, GCLM, GCN1,
GCNA, GCNT2, GCNT3, GCNT7, GCSH, GDA, GDAP1, GDAP2, GDF11, GDF15, GDF9,
GDI1, GDPD5, GEMIN4, GEMIN5, GEMIN6, GET1, GET3, GET4, GFI1, GFHB, GFOD1,
GFPT1, GFRA2, GGA1, GGA3, GGCX, GGPS1, GGT1, GGT6, GGT7, GHDC, GHITM,
GIGYF1, GIMAP4, GIMAP5, GIMAP7, GIN1, GINM1, GINS2, GINS3, GINS4, GIPC3,
GIPR, GIT1, GJB3, GJB5, GK, GK5, GKN2, GLB1L2, GLB1L3, GLCE, GLG1, GLI3,
GLI4, GLIPR1, GLIS3, GLO1, GLRX, GLRX2, GLRX3, GLRX5, GLT1D1, GLTP,
GLUD1, GLUD2, GLUL, GLYATL2, GLYCTK, GLYR1, GM2A, GMDS, GMIP, GMNC,
GMNN, GMPPB, GMPR, GMPR2, GNA11, GNA14, GNAI1, GNAI2, GNAI3, GNAL,
GNAO1, GNAZ, GNB1, GNB1L, GNB2, GNB4, GNB5, GNG12, GNG2, GNG5, GNG7,
GNGT2, GNL1, GNL2, GNLY, GNPDA1, GNPDA2, GNPNAT1, GNPTAB, GNPTG,
GOLGA1, GOLGA2, GOLGA3, GOLGA4, GOLGA5, GOLGA6L10, GOLGA7B,
GOLGA8A, GOLGA8B, GOLGA8N, GOLGA8R, GOLGB1, GOLIM4, GOLM1, GOLPH3,
GOLPH3L, GOLT1B, GORAB, GORASP1, GOSR1, GP1BA, GP5, GP6, GP9, GPAA1,
GPALPP1, GPANK1, GPAT2, GPAT3, GPAT4, GPATCH2, GPATCH2L, GPATCH8,
GPBAR1, GPBP1, GPBP1L1, GPC2, GPC4, GPCPD1, GPD1, GPD1L, GPD2, GPER1,
GPN2, GPN3, GPNMB, GPR107, GPR108, GPR132, GPR137, GPR137B, GPR141,
GPR146, GPR153, GPR160, GPR162, GPR171, GPR174, GPR176, GPR183, GPR22, GPR3,
GPR34, GPR35, GPR45, GPR55, GPR65, GPR68, GPR75, GPR82, GPR84, GPR85, GPR87,
GPR89A, GPR89B, GPRASP2, GPRC5A, GPRIN3, GPS1, GPS2, GPSM1, GPSM3, GPX2,
GPX3, GPX8, GRAMD1A, GRAMD1C, GRAMD2A, GRAMD2B, GRAMD4, GRAP,
GRAP2, GRAPL, GRASP, GRB10, GRB2, GRB7, GREB1, GRHL1, GRHL2, GRHL3,
GRHPR, GRIK2, GRIN2C, GRIN3A, GRINA, GRIP1, GRIPAP1, GRK2, GRK3, GRK5,
GRK6, GRM2, GRN, GRWD1, GSDMA, GSDMB, GSDMC, GSDMD, GSE1, GSK3A,
GSK3B, GSKIP, GSTA1, GSTA2, GSTA3, GSTA5, GSTCD, GSTK1, GSTM1, GSTM2,
GSTM3, GST01, GSTT2, GSTT2B, GSTZ1, GTDC1, GTF2A1, GTF2B, GTF2E1, GTF2F1,
GTF2F2, GTF2H1, GTF2H2, GTF2H2C, GTF2H5, GTF2IRD1, GTF3A, GTF3C1, GTF3C2,
GTF3C4, GTF3C5, GTF3C6, GTPBP1, GTPBP3, GTPBP6, GTSF1, GUCD1, GUCY1B1,
GUCY2C, GUK1, GULP1, GUSB, GXYLT1, GXYLT2, GYPA, GYPB, GYPC, GYS1,
GZF1, GZMB, GZMH, GZMK, GZMM, H1-0, H1-1, H1-10, H1-3, H1-4, H1-5, H19,
H2AC11, H2AC13, H2AC14, H2AC16, H2AC18, H2AC19, H2AC20, H2AC21, H2AC8,
H2AJ, H2AW, H2AZ1, H2AZ2, H2BC12, H2BC18, H2BC7, H3-3A, H3-3B, H3-5, H3C10,
H3C12, H3C6, H4C12, H4C13, H4C14, H4C15, H4C2, H4C3, H4C5, H4C9, H6PD,
HACD3, HADH, HADHA, HAGH, HAGHL, HAL, HAPLN3, HARB11, HARS1, HARS2,
HAT1, HAUS2, HAUS3, HAUS4, HAUS5, HAUS8, HAVCR2, HAX1, HBA1, HBA2, HBB,
HBD, HBEGF, HBG1, HBG2, HBM, HBP1, HBQ1, HBS1L, HCAR2, HCFC1, HCFC2,
HCK, HCLS1, HDAC1, HDAC10, HDAC11, HDAC3, HDAC4, HDAC5, HDAC6, HDAC7,
HDAC8, HDAC9, HDDC2, HDGF, HDGFL2, HDGFL3, HDHD2, HDHD5, HDX,
HEATR1, HEATR3, HEATR5A, HEATR6, HECA, HECTD1, HECTD2, HECTD4,
HECW2, HELQ, HELZ2, HEMGN, HEMK1, HERC2, HERC4, HERC6, HERPUD1,
HERPUD2, HES1, HES2, HESX1, HEXD, HEXIM1, HEXIM2, HEY1, HEY2, HFE, HGH1,
HGS, HGSNAT, HHLA2, HIBADH, HIBCH, HIC1, HIC2, HIF1AN, HIGD1A, HIGD2A,
HIKESHI, HILPDA, HINT1, HINT3, HIP1R, HIPK1, HIPK2, HIRIP3, HIVEP1, HJURP,
HJV, HK1, HK3, HLA-A, HLA-B, HLA-C, HLA-DOB, HLA-DPA1, HLA-DQA2, HLA-
DQB2, HLA-E, HLA-F, HLA-G, HLCS, HLF, HLTF, HLX, HM13, HMBOX1, HMBS,
HMCES, HMG20B, HMGA1, HMGB1, HMGB2, HMGCR, HMGCS2, HMGN3, HMGN4,
HMGN5, HMGXB3, HNF1B, HNMT, HNRNPA0, HNRNPA1, HNRNPA1L2,
HNRNPA2B1, HNRNPA3, HNRNPAB, HNRNPC, HNRNPD, HNRNPH1, HNRNPH2,
HNRNPH3, HNRNPK, HNRNPL, HNRNPM, HNRNPR, HNRNPU, HNRNPUL1,
HOMER1, HOMER2, HOMEZ, HOOK1, HOOK3, HOPX, HOXB2, HOXB3, HOXB6,
HPCAL1, HPCAL4, HPGD, HPGDS, HPS1, HPS4, HPS5, HPS6, HPSE, HRH2, HS1BP3,
HS2ST1, HS3ST1, HS3ST2, HS3ST3B1, HS6ST1, HSBP1, HSBP1L1, HSD11B1,
HSD17B1, HSD17B14, HSD17B4, HSD3B7, HSDL1, HSDL2, HSF1, HSF2, HSF4, HSH2D,
HSP90AA1, HSP90AB1, HSPA1A, HSPA1B, HSPA4, HSPA4L, HSPA5, HSPA8, HSPB1,
HSPB11, HSPB8, HSPBAP1, HSPBP1, HSPD1, HSPE1, HSPG2, HSPH1, HTATIP2,
HTATSF1, HTN1, HTN3, HTRA2, HTRA3, HTRA4, HTT, HUNK, HVCN1, HYDIN,
HYKK, HYOU1, HYPK, IAH1, IARS2, IBA57, IBTK, ICAM1, ICAM2, ICAM3, ICAM5,
ICE2, ICK, ICMT, ICOSLG, ID1, ID2, ID3, ID4, IDH1, IDH2, IDH3A, IDH3B, IDO1,
IDUA, IER2, IER5, IER5L, IFFO1, IFFO2, IFI16, IFI27L2, IFI6, IFIH1, IFITI, IFIT2, IFIT3,
IFIT5, IFITM1, IFITM3, IFNG, IFNGR2, IFRD1, IFRD2, IFT22, IFT43, IFT74, IFT81,
IGF1, IGF1R, IGF2BP3, IGF2R, IGFBP3, IGFBP5, IGFLR1, IGHMBP2, IGIP, IGLL5,
IGSF10, IGSF3, IGSF6, IGSF8, IGSF9B, IK, IKBKB, IKBKE, IKBKG, IKZF1, IKZF2,
IKZF3, IKZF5, IL10RA, IL11RA, IL12A, IL12RB1, IL12RB2, IL16, IL17C, IL17RA,
IL17RB, IL17RC, IL17RD, IL18, IL18RAP, IL19, IL1A, IL1B, IL1R1, IL1R2, IL1RN,
IL20RA, IL20RB, IL21R, IL22RA1, IL24, IL26, IL27RA, IL2RA, IL2RB, IL2RG, IL32,
IL33, IL36B, IL36G, IL36RN, IL4I1, IL4R, IL5RA, IL6R, IL7R, ILF2, ILF3, ILK, ILKAP,
ILRUN, ILVBL, IMMT, IMPA1, IMPA2, IMPACT, IMPAD1, IMPDH1, INAFM1, INCA1,
INF2, ING1, ING2, ING3, ING4, ING5, INHBA, INIP, INKA1, INKA2, INO80, INO80C,
INO80D, INO80E, INPP4A, INPP5A, INPP5B, INPP5D, INPP5F, INPP5K, INPPL1, INSL6,
INSR, INSYN2A, INTS1, INTS11, INTS12, INTS2, INTS3, INTS5, INTS6, INTS6L,
INTS7, INTS8, INTS9, INTU, INVS, IP6K1, IP6K2, IPCEF1, IPO11, IPO13, IPO7, IPO8,
IPPK, IQCA1, IQCE, IQCG, IQCH, IQCK, IQCN, IQGAP3, IQSEC1, IQUB, IRAK1,
IRAK1BP1, IRAK2, IRAK3, IRF1, IRF2, IRF2BP1, IRF2BP2, IRF2BPL, IRF3, IRF5, IRF6,
IRF8, IRF9, IRGQ, IRS1, IRS2, IRX3, ISCA2, ISG15, ISG20, ISG20L2, ISL1, ISOC2, IST1,
ISY1, ISYNA1, ITCH, ITGA10, ITGA2, ITGA2B, ITGA3, ITGA4, ITGA5, ITGA6, ITGA7,
ITGA8, ITGAL, ITGAM, ITGAV, ITGAX, ITGB1, ITGB2, ITGB3, ITGB4, ITGB6, ITGB7,
ITGB8, ITIH5, ITK, ITM2A, ITM2B, ITM2C, ITPA, ITPKB, ITPR2, ITPR3, ITPRID2,
ITPRIP, ITPRIPL1, ITPRIPL2, ITSN1, IWS1, IYD, IZUMO4, JADE1, JADE2, JADE3,
JAG1, JAGN1, JAK1, JAK2, JAK3, JAKMIP1, JAKMIP2, JAM3, JAML, JDP2, JHY,
JMJD1C, JMJD6, JMJD7-PLA2G4B, JMJD8, JOSD1, JPH4, JPT1, JPT2, JRK, JTB, JUN,
JUNB, JUND, KALRN, KANK2, KANSL1-AS1, KANSL1L, KARS1, KAT2A, KAT5,
KAT7, KAT8, KATNAL1, KATNAL2, KATNB1, KATNBL1, KAZALD1, KBTBD11,
KBTBD2, KBTBD3, KBTBD8, KCNA10, KCNA2, KCNAB1, KCNAB2, KCNAB3,
KCNC4, KCND1, KCNE1B, KCNH2, KCNH3, KCNH8, KCNIP2, KCNJ1, KCNJ10,
KCNJ13, KCNJ16, KCNJ5, KCNK1, KCNK13, KCNK6, KCNK7, KCNMA1, KCNMB2,
KCNN4, KCNQ1, KCNQ3, KCTD1, KCTD11, KCTD12, KCTD13, KCTD17, KCTD20,
KCTD7, KCTD9, KDELR1, KDELR2, KDELR3, KDF1, KDM2A, KDM3B, KDM4B,
KDM4C, KDM5A, KDM5C, KDM5D, KDM6A, KDM6B, KDM7A, KEL, KHDC4,
KHDRBS1, KHDRBS3, KHNYN, KHSRP, KIAA0100, KIAA0232, KIAA0319L,
KIAA0355, KIAA0513, KIAA0556, KIAA0586, KIAA0825, KIAA0895, KIAA0895L,
KIAA0930, KIAA1143, KIAA1211L, KIAA1217, KIAA1324, KIAA1328, KIAA1522,
KIAA1614, KIAA1671, KIAA1841, KIAA1958, KIAA2012, KIAA2013, KIAA2026,
KIF16B, KIF20B, KIF21A, KIF21B, KIF22, KIF24, KIF26A, KIF2C, KIF3A, KIF3B,
KIF5B, KIFBP, KIFC2, KIN, KIT, KIZ, KLC1, KLC2, KLC3, KLC4, KLF1, KLF10,
KLF11, KLF12, KLF13, KLF15, KLF16, KLF2, KLF4, KLF5, KLF6, KLF9, KLHDC2,
KLHDC3, KLHDC4, KLHDC8A, KLHDC8B, KLHDC9, KLHL11, KLHL15, KLHL17,
KLHL18, KLHL21, KLHL22, KLHL25, KLHL26, KLHL28, KLHL29, KLHL3, KLHL36,
KLHL41, KLHL6, KLK1, KLK10, KLK11, KLK13, KLRB1, KLRC2, KLRC3, KLRD1,
KLRF1, KLRG1, KLRK1, KMO, KMT2B, KMT2C, KMT2D, KMT2E, KMT5C, KNDC1,
KPNA1, KPNA3, KPNA4, KPNA6, KPNA7, KPRP, KPTN, KRBA1, KRBOX4, KRCC1,
KRIT1, KRT1, KRT10, KRT13, KRT15, KRT16, KRT17, KRT18, KRT19, KRT23, KRT4,
KRT5, KRT6A, KRT6B, KRT6C, KRT7, KRT72, KRT73, KRT78, KRT8, KRT80,
KRTCAP2, KSR1, KTI12, KXD1, KYAT3, KYNU, L3MBTL1, L3MBTL2, LACC1,
LACTB, LACTB2, LAD1, LAIR2, LAMA1, LAMA2, LAMA3, LAMA5, LAMB1, LAMB2,
LAMB3, LAMC1, LAMP3, LAMTOR1, LAMTOR2, LAMTOR3, LAMTOR4, LAMTOR5,
LANCL3, LAP3, LAPTM5, LARGE2, LARP1B, LARP4, LARP7, LARS1, LARS2, LAS1L,
LASP1, LAT, LAT2, LBH, LBHD1, LBR, LCA5, LCA5L, LCAT, LCE3A, LCE3D, LCE3E,
LCK, LCLAT1, LCMT2, LCN2, LCORL, LCP1, LDB1, LDHAL6B, LDHB, LDHD, LDLR,
LDLRAD1, LDLRAD2, LDLRAP1, LDOC1, LEF1, LEKR1, LEMD2, LEMD3, LENG8,
LEO1, LEP, LEPROT, LEPROTL1, LETM1, LETM2, LETMD1, LFNG, LGALS2,
LGALS3, LGALS9B, LGMN, LGR4, LGR6, LHFPL2, LHFPL6, LHPP, LHX4, LIFR, LIG1,
LIG3, LIG4, LILRA1, LILRA2, LILRA5, LILRA6, LILRB1, LILRB2, LILRB4, LIMA1,
LIMCH1, LIMD1, LIMD2, LIMK1, LIMK2, LIMS1, LIN37, LIN54, LIN7C, LIN9,
LINC01410, LINC02701, LINGO3, LIPE, LITAF, LKAAEAR1, LLGL1, LLGL2, LMAN2,
LMBR1, LMBR1L, LMCD1, LMF1, LMF2, LMLN, LMNA, LMNTD1, LMNTD2, LMO3,
LMO4, LMO7, LMTK2, LMTK3, LNP1, LNX1, LONP1, LONRF2, LOX, LOXHD1,
LOXL1, LPAR1, LPAR2, LPAR3, LPAR5, LPCAT1, LPCAT3, LPCAT4, LPGAT1, LPIN2,
LPIN3, LPL, LPXN, LRATD1, LRBA, LRCH3, LRCH4, LRFN1, LRFN4, LRG1, LRIF1,
LRIG1, LRIG2, LRIG3, LRMDA, LRMP, LRP1, LRP10, LRP11, LRP2BP, LRP3, LRP5L,
LRR1, LRRC1, LRRC14, LRRC18, LRRC20, LRRC23, LRRC25, LRRC3, LRRC34,
LRRC36, LRRC37A3, LRRC37B, LRRC41, LRRC42, LRRC45, LRRC46, LRRC47,
LRRC49, LRRC58, LRRC6, LRRC63, LRRC71, LRRC74A, LRRC8A, LRRCC1, LRRFIP1,
LRRIQ1, LRRK1, LRRK2, LRRN1, LRRN2, LRRN3, LRWD1, LSAMP, LSG1, LSM12,
LSM14B, LSM3, LSM7, LSP1, LSR, LSS, LTA, LTB, LTB4R, LTB4R2, LTBP1, LTBP3,
LTBP4, LTBR, LTK, LTO1, LUC7L, LUC7L2, LUC7L3, LURAP1L, LVRN, LXN, LY6D,
LY6E, LY6G5B, LY9, LYL1, LYPD2, LYPD5, LYPD6B, LYPLA2, LYRM2, LYRM4,
LYRM9, LYSMD1, LYSMD2, LYSMD3, LYSMD4, LYST, LYVE1, LZTFL1, LZTS2,
LZTS3, M1AP, M6PR, MAATS1, MAB21L4, MACC1, MACROH2A1, MACROH2A2,
MAD1L1, MAD2L1BP, MAD2L2, MADD, MAEA, MAF1, MAFF, MAFG, MAFK,
MAGEE1, MAGEH1, MAGI1, MAGI3, MAGOH, MAGOHB, MAIP1, MAK16, MAL2,
MALAT1, MALL, MALSU1, MALT1, MAMDC4, MAML2, MAMLD1, MAN1A1,
MAN1B1, MAN2A1, MAN2A2, MAN2B1, MAN2B2, MAN2C1, MANEA, MANEAL,
MANF, MANSC4, MAP11, MAP1A, MAP1B, MAP1LC3C, MAP1S, MAP2K2, MAP2K3,
MAP2K5, MAP2K6, MAP2K7, MAP3K10, MAP3K11, MAP3K12, MAP3K14, MAP3K19,
MAP3K3, MAP3K4, MAP3K6, MAP3K7, MAP4, MAP4K1, MAP4K2, MAP4K3,
MAP4K4, MAP4K5, MAP6, MAP7, MAP7D1, MAP9, MAPK10, MAPK11, MAPK14,
MAPK1IP1L, MAPK3, MAPK6, MAPK8IP3, MAPKAP1, MAPKAPK3, MAPKBP1,
MAPRE1, MARC1, MARCHF1, MARCHF2, MARCHF6, MARCHF8, MARCHF9,
MARCO, MARF1, MARK2, MARS1, MARS2, MARVELD1, MARVELD2, MARVELD3,
MASP2, MAST3, MASTL, MATK, MAU2, MAVS, MAZ, MB, MBD1, MBD3, MBD4,
MBD6, MBLAC1, MBNL1, MBNL2, MBOAT7, MBP, MBTD1, MBTPS1, MC1R, MCC,
MCCC1, MCF2, MCFD2, MCIDAS, MCL1, MCM2, MCM3AP, MCM3AP-AS1, MCM4,
MCM5, MCM7, MCMBP, MCOLN1, MCPH1, MCR1P1, MCR1P2, MCRS1, MCTP1,
MCTP2, MCTS1, MCU, MCUR1, MDGA1, MDH1, MDH1B, MDM2, MDM4, ME1, ME2,
ME3, MECOM, MECP2, MED10, MED11, MED12, MED13, MED13L, MED14, MED15,
MED16, MED18, MED19, MED21, MED22, MED24, MED25, MED26, MED28, MED30,
MED7, MED8, MEF2D, MEFV, MEGF11, MEGF6, MEGF8, MEGF9, MEIS2, MEIS3,
MEMO1, MEN1, MEP1A, MEPCE, MEST, MET, METAP1, METAP1D, METAP2,
METTL14, METTL17, METTL21A, METTL23, METTL25, METTL3, METTL4, METTL5,
METTL6, METTL7B, METTL8, METTL9, MEX3C, MFAP1, MFAP5, MFGE8, MFN2,
MFNG, MFSD10, MFSD11, MFSD12, MFSD13A, MFSD14A, MFSD14B, MFSD14C,
MFSD2A, MFSD2B, MFSD3, MFSD4A, MFSD4B, MFSD6, MFSD8, MGA, MGAM,
MGAT1, MGAT4A, MGAT4B, MGLL, MGRN1, MGST2, MGST3, MIB2, MICAL1,
MICAL2, MICAL3, MICALL1, MICALL2, MICOS13, MICU2, MID1, MID1IP1, MIDN,
MIEN1, MIER1, MIF4GD, MIGA2, MIIP, MINCR, MINDY2, MINK1, MIPOL1,
MIR155HG, MIR421, MIR4435-2HG, MIS12, MITF, MKKS, MKNK2, MKRN1, MLC1,
MLEC, MLF1, MLF2, MLH1, MLLT1, MLLT11, MLLT3, MLLT6, MLPH, MLST8, MLX,
MLXIP, MLYCD, MMAA, MMAB, MMADHC, MME, MMGT1, MMP1, MMP10,
MMP12, MMP17, MMP19, MMP23B, MMP24, MMP25, MMP7, MMP9, MMRN1,
MMS19, MMS22L, MMUT, MNAT1, MNS1, MNT, MOAP1, MOB1A, MOB2, MOB3A,
MOB3B, MOB3C, MOB4, MOCOS, MOCS2, MOCS3, MOGS, MOK, MORC4, MORN1,
MORN2, MORN3, MORN5, MOSPD1, MOSPD2, MOSPD3, MOV10, MPC1, MPC2,
MPDU1, MPDZ, MPEG1, MPG, MPHOSPH6, MPIG6B, MPO, MPP1, MPP5, MPPE1,
MPRIP, MPST, MPV17, MPV17L, MPV17L2, MPZL2, MR1, MRAP2, MRAS, MRC1,
MRFAP1, MRFAP1L1, MRI1, MRM2, MRNIP, MRO, MROH1, MRPL1, MRPL13,
MRPL17, MRPL18, MRPL19, MRPL2, MRPL20, MRPL21, MRPL23, MRPL27, MRPL28,
MRPL32, MRPL33, MRPL34, MRPL35, MRPL36, MRPL37, MRPL38, MRPL39, MRPL4,
MRPL40, MRPL42, MRPL44, MRPL45, MRPL46, MRPL48, MRPL50, MRPL51, MRPL54,
MRPL57, MRPL58, MRPS10, MRPS12, MRPS14, MRPS16, MRPS18B, MRPS18C,
MRPS2, MRPS23, MRPS25, MRPS26, MRPS30, MRPS31, MRPS33, MRPS34, MRPS35,
MRPS36, MRPS5, MRTFA, MRTFB, MS4A1, MS4A14, MS4A2, MS4A3, MS4A4A,
MS4A8, MSANTD3, MSANTD4, MSH3, MSH5, MSH6, MSI2, MSL1, MSL2, MSL3,
MSLN, MSMB, MSN, MSR1, MSRB1, MSRB3, MSS51, MSTO1, MT1E, MT1F, MT1G,
MT1H, MT1X, MT2A, MT3, MTA1, MTA2, MTAP, MT-ATP6, MT-ATP8, MTBP,
MTCH1, MTCH2, MT-CO1, MT-CO2, MT-CO3, MT-CYB, MTDH, MTERF1, MTF2,
MTFR1L, MTG1, MTHFD1L, MTHFD2, MTHFD2L, MTHFR, MTHFSD, MTIF2, MTIF3,
MTLN, MTMR11, MTMR14, MTMR2, MTMR3, MTMR9, MT-ND1, MT-ND2, MT-ND3,
MT-ND4, MT-ND4L, MT-ND5, MT-ND6, MTPN, MTR, MTREX, MTRF1, MTRNR2L1,
MTRNR2L10, MTRNR2L12, MTRNR2L3, MTRNR2L5, MTRNR2L6, MTRNR2L7,
MTRNR2L8, MTRR, MTUS1, MTX1, MTX2, MTX3, MUC1, MUC13, MUC15, MUC16,
MUC2, MUC20, MUC21, MUC22, MUC4, MUC5AC, MUC5B, MUC7, MUL1, MUS81,
MUTYH, MVB12A, MVD, MVK, MVP, MX1, MXD3, MXD4, MXI1, MXRA7, MXRA8,
MYADM, MYB, MYBBP1A, MYBL1, MYBL2, MYCBP2, MYCBPAP, MYCL, MYDGF,
MYEF2, MYH14, MYH9, MYL12A, MYL12B, MYL4, MYL5, MYL6, MYL6B, MYL9,
MYLIP, MYLK, MYLK4, MYNN, MYO10, MYO15A, MYO15B, MYO18A, MYO19,
MYO1B, MYO1C, MYO1D, MYO1E, MYO1F, MYO1G, MYO5A, MYO5B, MYO5C,
MYO6, MYO9A, MYO9B, MYOF, MYOM1, MYOM2, MYPOP, MYRF, MYRIP,
MYZAP, MZF1, MZT2B, N4BP1, N4BP2L2, N4BP3, N6AMT1, NAA15, NAA16, NAA25,
NAA40, NAA50, NAA80, NAAA, NAALADL2, NAB1, NABP1, NACA, NACA2, NACC1,
NACC2, NADSYN1, NAGA, NAGK, NAGPA, NAIF1, NAIP, NANP, NAP1L4, NAPA,
NAPB, NAPEPLD, NAPRT, NARF, NARS1, NARS2, NAT14, NAT8B, NAT9, NATD1,
NAV2, NAXD, NBAS, NBDY, NBEA, NBEAL1, NBEAL2, NBN, NBPF10, NBPF11,
NBPF12, NBPF14, NBPF15, NBPF19, NBPF20, NBPF26, NBPF3, NBPF8, NBPF9, NBR1,
NCALD, NCAM1, NCAPD2, NCAPD3, NCAPG2, NCAPH2, NCBP2, NCBP3, NCCRP1,
NCEH1, NCF1, NCF2, NCK1, NCK2, NCKAP1, NCKAP5, NCKAP5L, NCL, NCLN,
NCMAP, NCOA2, NCOA5, NCOA7, NCOR1, NCOR2, NCR1, NCR3, NCS1, NCSTN,
NDE1, NDEL1, NDFIP1, NDFIP2, NDN, NDNF, NDOR1, NDRG1, NDRG2, NDST1,
NDST2, NDUFA1, NDUFA10, NDUFA12, NDUFA13, NDUFA2, NDUFA3, NDUFA4,
NDUFA5, NDUFA6, NDUFA8, NDUFAB1, NDUFAF2, NDUFAF3, NDUFB1, NDUFB2,
NDUFB3, NDUFB4, NDUFB6, NDUFB7, NDUFB8, NDUFB9, NDUFC1, NDUFS3,
NDUFS4, NDUFS5, NDUFS6, NDUFS7, NDUFV1, NDUFV2, NDUFV3, NEBL, NECAB3,
NECAP1, NECTIN4, NEDD9, NEIL1, NEIL3, NEK1, NEK10, NEK11, NEK2, NEK4,
NEK5, NEK6, NEK7, NEK8, NEK9, NELFB, NELFCD, NELFE, NEMF, NEMP1, NEMP2,
NENF, NEO1, NET1, NETO1, NEU4, NEURL1, NEURL1B, NEURL2, NEURL3,
NEURL4, NEXMIF, NF1, NFAM1, NFAT5, NFATC1, NFATC2, NFATC2IP, NFE2,
NFE2L1, NFE2L2, NFIA, NFIB, NFIC, NFIL3, NFKB2, NFKBIA, NFKBIB, NFKBID,
NFKBIE, NFKBIL1, NFKBIZ, NFS1, NFX1, NFXL1, NFYC, NGEF, NGRN, NHLRC2,
NHP2, NHS, NHSL1, NHSL2, NIBAN2, NIBAN3, NIDI, NIF3L1, NIFK, NIN, NINJ2,
NINL, NIP7, NIPBL, NISCH, NIT1, NKAP, NKAPD1, NKG7, NKIRAS1, NKIRAS2,
NKTR, NKX2-1, NKX3-1, NLGN2, NLGN3, NLK, NLRC3, NLRC4, NLRC5, NLRP1,
NLRP12, NLRP3, NLRP6, NLRX1, NMB, NMD3, NME1, NME2, NME3, NME4, NME5,
NME7, NME9, NMI, NMNAT2, NMRAL1, NMT1, NMT2, NMUR1, NNAT, NNT,
NOC2L, NOC4L, NOCT, NOD2, NOL4L, NOL6, NOL7, NOL9, NOM1, NOMO1, NOMO2,
NOMO3, NONO, NOP10, NOP53, NOP56, NOP9, NOS2, NOSIP, NOTCH1, NOTCH2,
NOTCH2NLA, NOTCH4, NOX4, NOXA1, NPAS2, NPAS3, NPAT, NPEPL1, NPIPA1,
NPIPA2, NPIPA3, NPIPA5, NPIPA7, NPIPA8, NPIPB12, NPIPB13, NPIPB15, NPIPB2,
NPIPB3, NPIPB4, NPIPB5, NPIPB6, NPIPB9, NPL, NPLOC4, NPM3, NPNT, NPRL3,
NPTN, NPTXR, NPW, NQO1, NR1H2, NR1H3, NR2F2, NR2F6, NR3C1, NR4A1, NR4A2,
NRAS, NRBF2, NRBP2, NRCAM, NRDC, NRG1, NRG4, NRGN, NRIP1, NRIP2, NRM,
NRP1, NRP2, NRROS, NRXN1, NRXN3, NSA2, NSD1, NSF, NSFL1C, NSG1, NSL1,
NSMAF, NSMCE1, NSMCE3, NSMF, NSRP1, NSUN2, NSUN5, NSUN6, NSUN7, NT5C2,
NT5DC2, NT5DC3, NT5M, NTAN1, NTF4, NTMT1, NTN4, NTNG2, NTRK3, NTSR1,
NUAK1, NUAK2, NUBP1, NUBP2, NUBPL, NUCB1, NUCKS1, NUDCD1, NUDCD2,
NUDCD3, NUDT12, NUDT14, NUDT15, NUDT16L1, NUDT17, NUDT19, NUDT2,
NUDT21, NUDT22, NUDT3, NUDT5, NUDT8, NUFIP1, NUFIP2, NUGGC, NUMA1,
NUP188, NUP205, NUP210, NUP42, NUP50, NUP54, NUP58, NUP62, NUP85, NUP88,
NUP93, NUPR1, NUS1, NUTM2A, NUTM2D, NUTM2G, NWD1, NXF1, NXF3, NXN,
NXPE3, OAF, OAS1, OAS2, OASL, OAT, OAZ1, OAZ2, OBI1, OBSCN, OCIAD1, OCLN,
OCRL, ODF2, ODF2L, ODF3B, OFD1, OGA, OGDH, OGFOD1, OGFOD3, OGFR, OGG1,
OGT, OLA1, OLAH, OLFML2A, OLFML2B, OLFML3, OLIG1, OLR1, OMA1, OMD,
OMG, OPA3, OPHN1, OPLAH, OPRL1, OR2A4, OR2A42, OR2A7, OR2B6, OR2W3,
OR5AU1, ORAI1, ORAI2, ORAI3, ORC1, ORC2, ORC3, ORM2, ORMDL1, ORMDL2,
ORMDL3, OS9, OSBP2, OSBPL11, OSBPL1A, OSBPL2, OSBPL5, OSBPL6, OSBPL7,
OSBPL8, OSBPL9, OSCP1, OSER1, OSGEP, OSGIN2, OSMR, OSR2, OTOA, OTUB1,
OTUB2, OTUD1, OTUD3, OTUD6B, OTUD7B, OTULIN, OTX1, OVCH1, OVGP1,
OXA1L, OXER1, OXLD1, OXNAD1, OXSR1, P2RX1, P2RX4, P2RX5, P2RY10, P2RY13,
P2RY8, P3H1, P4HA1, P4HA2, P4HB, P4HTM, PABPC1L, PABPC3, PABPN1, PACRG,
PACS1, PACS2, PACSIN1, PACSIN2, PADI1, PADI4, PAFAH1B1, PAFAH1B2, PAG1,
PAGE2B, PAIP2, PAK1, PAKHP1, PAK4, PALB2, PALLD, PALMD, PAM16, PAN2,
PAN3, PANK1, PANK2, PANK4, PANX2, PAPLN, PAPSS1, PAPSS2, PAQR4, PAQR5,
PAQR6, PAQR7, PAQR8, PARD3, PARD3B, PARD6A, PARD6B, PARG, PARK7, PARE,
PARP1, PARP10, PARP11, PARP14, PARP15, PARP2, PARP4, PARP8, PARVA, PARVB,
PARVG, PASK, PATL2, PATZ1, PAX5, PAX9, PAXBP1, PBDC1, PBLD, PBRM1, PBX2,
PBX3, PBX4, PBXIP1, PCBD1, PCBP1, PCBP2, PCBP4, PCCA, PCDH12, PCDH9,
PCDHGB7, PCED1A, PCED1B, PCGF3, PCGF6, PCIF1, PCLO, PCM1, PCMT1, PCMTD2,
PCNA, PCNP, PCNT, PCNX1, PCNX3, PCOLCE2, PCP4L1, PCSK1N, PCSK5, PCSK6,
PCSK7, PCYOX1, PCYOX1L, PDAP1, PDC, PDCD1, PDCD10, PDCD11, PDCD1LG2,
PDCD2L, PDCD6, PDCL3, PDE2A, PDE3A, PDE4D, PDE4DIP, PDE5A, PDE6B, PDE6D,
PDE7A, PDE8A, PDE8B, PDE9A, PDGFA, PDGFC, PDGFRB, PDHA1, PDHX, PDIA3,
PDIA4, PDIA5, PDIA6, PDK1, PDK3, PDK4, PDLIM1, PDLIM2, PDLIM3, PDLIM5,
PDLIM7, PDP1, PDPR, PDRG1, PDS5B, PDSS1, PDXDC1, PDZD11, PDZD2, PDZD4,
PDZD8, PDZK1, PDZK1IP1, PEAK1, PEAK3, PEAR1, PEBP1, PEBP4, PEG10, PEG3,
PELI1, PELI2, PELP1, PEMT, PER1, PER2, PER3, PERP, PES1, PET100, PEX1, PEX10,
PEX11B, PEX13, PEX16, PEX26, PF4, PF4V1, PFDN1, PFDN2, PFDN5, PFDN6, PFKFB3,
PFKFB4, PFKL, PFKM, PFKP, PFN1, PFN2, PFN4, PGA3, PGA4, PGA5, PGAM1,
PGAM4, PGAM5, PGAP2, PGAP3, PGAP4, PGAP6, PGBD2, PGBD4, PGD, PGGHG,
PGGT1B, PGK1, PGM2, PGM2L1, PGM3, PGM5, PGPEP1, PGRMC1, PGS1, PHACTR1,
PHACTR2, PHACTR4, PHAX, PHB2, PHC2, PHC3, PHETA1, PHEX, PHF1, PHF10,
PHF12, PHF13, PHF14, PHF19, PHF2, PHF21A, PHF3, PHF5A, PHF6, PHF8, PHKG2,
PHLDA1, PHLDA3, PHLDB3, PHLPP1, PHOSPHO1, PHRF1, PHTF2, PHYH, PHYHD1,
PHYKPL, PI3, PI4K2A, PI4K2B, PI4KA, PI4KB, PIAS1, PIAS2, PIAS3, PIAS4, PIBF1,
PICALM, PICK1, PID1, PIDD1, PIEZO1, PIF1, PIFO, PIGA, PIGF, PIGG, PIGK, PIGL,
PIGM, PIGO, PIGP, PIGQ, PIGR, PIGS, PIGT, PIGU, PIGX, PIGZ, PIH1D1, PIH1D2,
PIK3C2A, PIK3C2B, PIK3CD, PIK3IP1, PIK3R3, PIK3R5, PIK3R6, PILRB, PIM1, PIM2,
PIM3, PIN1, PIN4, PINK1, PIP, PIP4K2A, PIP4K2B, PIP4K2C, PIP4P1, PIP4P2, PIP5K1A,
PIP5K1C, PIP5KL1, PIR, PITPNA, PITPNB, PITPNC1, PITPNM1, PITPNM2, PITX1,
PIWIL4, PJA2, PKD1, PKD1L3, PKD2, PKD2L1, PKDCC, PKHD1L1, PKIB, PKM,
PKMYT1, PKN1, PKNOX1, PKP1, PKP2, PKP3, PKP4, PLA1A, PLA2G10, PLA2G4C,
PLA2G6, PLA2G7, PLA2R1, PLAAT2, PLAGL2, PLAT, PLAU, PLAUR, PLBD1, PLCB1,
PLCB2, PLCB4, PLCD1, PLCD3, PLCE1, PLCG1, PLCG2, PLCH1, PLCH2, PLCL2,
PLCXD1, PLD1, PLD2, PLD3, PLD4, PLD6, PLEC, PLEK, PLEK2, PLEKHA3, PLEKHA5,
PLEKHA7, PLEKHF1, PLEKHF2, PLEKHG2, PLEKHG3, PLEKHJ1, PLEKHM1,
PLEKHM2, PLEKHM3, PLEKHO2, PLEKHS1, PLGLB1, PLGLB2, PLGRKT, PLIN2,
PLIN3, PLIN4, PLK1, PLK2, PLK3, PLOD1, PLOD3, PLP2, PLPP2, PLPP3, PLPP6,
PLPPR2, PLS1, PLS3, PLSCR4, PLVAP, PLXDC1, PLXDC2, PLXNA1, PLXNA3,
PLXNA4, PLXNB2, PLXNB3, PLXNC1, PLXND1, PMEPA1, PMF1, PMFBP1, PMM1,
PMM2, PMP22, PMPCA, PMPCB, PMS1, PMVK, PNISR, PNKD, PNKP, PNMA3,
PNMA8A, PNN, PNP, PNPLA1, PNPLA2, PNPLA3, PNPLA4, PNPLA6, PNPLA8, PNPO,
POC1B, PODXL, POF1B, POFUT1, POFUT2, POGLUT1, POGLUT3, POLA1, POLA2,
POLB, POLDI, POLD3, POLD4, POLDIP2, POLDIP3, POLE, POLE3, POLG, POLG2,
POLK, POLL, POLM, POLR1A, POLR1B, POLR2A, POLR2B, POLR2C, POLR2E,
POLR2F, POLR2G, POLR2H, POLR2I, POLR2J2, POLR2J3, POLR2K, POLR2L,
POLR2M, POLR3B, POLR3C, POLR3E, POLR3F, POLR3H, POLR3K, POLRMT,
POM121, POM121C, POMGNT1, POMGNT2, POMK, POMP, POMT1, POMT2, POMZP3,
POP4, POPDC2, POR, POSTN, POT1, POTEE, POTEF, POTEI, POTEJ, POU2F1, POU2F2,
POU5F2, POU6F1, PP2D1, PPA1, PPA2, PPAN, PPARD, PPARG, PPARGC1A,
PPARGC1B, PPBP, PPCDC, PPCS, PPDPF, PPFIA1, PPFIBP1, PPHLN1, PPIB, PPIC,
PPID, PPIE, PPIF, PPIG, PPIL1, PPIL2, PPIL4, PPIL6, PPIP5K1, PPL, PPM1A, PPM1B,
PPM1D, PPM1F, PPM1G, PPM1H, PPM1K, PPM1L, PPM1M, PPOX, PPP1CA, PPP1CC,
PPP1R10, PPP1R12C, PPP1R13L, PPP1R14C, PPP1R15A, PPP1R15B, PPP1R16B,
PPP1R18, PPP1R1A, PPP1R1B, PPP1R32, PPP1R36, PPP1R3C, PPP1R3E, PPP1R3F,
PPP1R8, PPP1R9A, PPP1R9B, PPP2CA, PPP2CB, PPP2R1A, PPP2R1B, PPP2R2A,
PPP2R2B, PPP2R2D, PPP2R3A, PPP2R5A, PPP2R5B, PPP2R5D, PPP3CA, PPP3CC,
PPP3R1, PPP4C, PPP5C, PPP5D1, PPP6C, PPP6R1, PPP6R2, PPP6R3, PPRC1, PPT1,
PPT2, PPTC7, PPWD1, PQBP1, PRADC1, PRAF2, PRAG1, PRAM1, PRB1, PRB3, PRB4,
PRCC, PRDM1, PRDM10, PRDM15, PRDM2, PRDX1, PRDX2, PRDX3, PREB, PRELID1,
PRELID3A, PRELID3B, PREP, PREPL, PREX1, PRF1, PRH2, PRICKLE2, PRICKLE3,
PRIMPOL, PRKAAI, PRKAA2, PRKAB1, PRKAB2, PRKACA, PRKACB, PRKAG1,
PRKAG2, PRKAG3, PRKAR2A, PRKCA, PRKCB, PRKCD, PRKCH, PRKCI, PRKCQ,
PRKCSH, PRKD2, PRKD3, PRKN, PRKRA, PRKRIP1, PRMT1, PRMT2, PRMT6,
PRMT7, PRNP, PROCA1, PROCR, PROM1, PROM2, PRORP, PROS1, PROSER3,
PROX1, PRPF19, PRPF38B, PRPF39, PRPF40A, PRPF4B, PRPF6, PRPF8, PRPS1,
PRPSAP1, PRPSAP2, PRR11, PRR12, PRR13, PRR14, PRR15, PRR15L, PRR18, PRR29,
PRR5, PRR7, PRRC1, PRRC2A, PRRC2B, PRRC2C, PRRG3, PRRT4, PRRX1, PRRX2,
PRSS12, PRSS16, PRSS21, PRSS23, PRSS3, PRSS36, PRSS53, PRUNE1, PRUNE2, PRX,
PRXL2A, PRXL2B, PRXL2C, PSAP, PSCA, PSD, PSD3, PSD4, PSEN2, PSENEN, PSKH1,
PSMA2, PSMA3, PSMA4, PSMA6, PSMA7, PSMB1, PSMB10, PSMB2, PSMB5, PSMB7,
PSMB8, PSMC2, PSMC3IP, PSMC4, PSMC6, PSMD1, PSMD10, PSMD12, PSMD13,
PSMD14, PSMD2, PSMD3, PSMD5, PSMD8, PSME3, PSME4, PSMF1, PSMG1, PSMG2,
PSPC1, PSPH, PSRC1, PSTPIP1, PSTPIP2, PTAFR, PTAR1, PTBP1, PTBP2, PTBP3,
PTCD1, PTCRA, PTDSS1, PTDSS2, PTER, PTGDR, PTGDS, PTGER2, PTGER3,
PTGER4, PTGES, PTGES2, PTGES3L, PTGFR, PTGIR, PTGR1, PTGS1, PTK2, PTK2B,
PTMA, PTN, PTOV1, PTP4A1, PTP4A3, PTPA, PTPN12, PTPN13, PTPN14, PTPN18,
PTPN23, PTPN3, PTPN4, PTPN6, PTPN7, PTPRC, PTPRE, PTPRF, PTPRJ, PTPRK,
PTPRM, PTPRO, PTPRS, PTPRT, PTPRU, PTPRZ1, PTRH2, PTRHD1, PTX3, PUDP,
PUF60, PUM2, PUM3, PURA, PURB, PUS1, PUS10, PUS3, PWP1, PWP2, PWWP2B,
PWWP3A, PXDC1, PXDN, PXK, PXMP4, PXN, PXYLP1, PYCARD, PYCR2, PYGB,
PYGM, PYGO2, PYHIN1, PYM1, PYROXD2, PYURF, QARS1, QPCT, QPCTL, QRICH1,
QSER1, QSOX1, QSOX2, QTRT1, R3HDM4, RAB10, RAB11B, RAB11FIP1, RAB11FIP3,
RAB11FIP4, RAB12, RAB13, RAB1A, RAB1B, RAB20, RAB21, RAB22A, RAB23,
RAB24, RAB25, RAB27A, RAB30, RAB32, RAB35, RAB36, RAB37, RAB38, RAB39A,
RAB39B, RAB3C, RAB3D, RAB40B, RAB40C, RAB42, RAB43, RAB44, RAB5IF,
RAB6B, RAB7A, RAB7B, RAB8B, RAB9A, RABAC1, RABEP1, RABEP2, RABEPK,
RABGAP1, RABGAP1L, RABGEF1, RABGGTA, RABGGTB, RABIF, RABL2A,
RABL2B, RABL6, RAC1, RAC2, RAD17, RAD18, RAD23A, RAD51AP1, RAD51D,
RAD54L2, RAD9A, RADX, RAE1, RAF1, RAI1, RAI14, RAI2, RALBP1, RALGAPA2,
RALGAPB, RALGPS2, RALY, RAMAC, RAN, RANBP10, RANBP17, RANBP2,
RANBP6, RANBP9, RANGAP1, RAP1A, RAP1GAP, RAP2A, RAP2B, RAPGEF1,
RAPGEF4, RAPH1, RARA, RARB, RARG, RARRES1, RARS1, RARS2, RASA2, RASA3,
RASAL2, RASAL3, RASEF, RASGEF1A, RASGEF1B, RASGRP1, RASGRP2, RASGRP3,
RASGRP4, RASSF1, RASSF2, RASSF3, RASSF5, RASSF6, RASSF8, RASSF9, RAVER1,
RAVER2, RB1, RBBP4, RBBP6, RBBP8, RBBP9, RBCK1, RBFA, RBFOX2, RBL1, RBL2,
RBM10, RBM11, RBM12, RBM12B, RBM14, RBM14-RBM4, RBM15, RBM17, RBM18,
RBM19, RBM22, RBM23, RBM24, RBM27, RBM3, RBM33, RBM34, RBM38, RBM39,
RBM4, RBM41, RBM43, RBM44, RBM47, RBM5, RBM6, RBMS2, RBMS3, RBMX,
RBMXL1, RBP1, RBP4, RBP5, RBPJ, RBPMS, RBSN, RBX1, RCBTB2, RCC1, RCC1L,
RCE1, RCHY1, RCL1, RCN2, RCN3, RCSD1, RDH10, RDH11, RDH12, RDH13, RDH5,
RDX, REC8, REEP3, RELA, RELB, RELCH, RELL2, RELT, REM2, RENBP, REPIN1,
REPS1, RER1, RERE, RERG, RESF1, REST, RETREG1, RETREG2, RETREG3, RETSAT,
REV3L, REX1BD, REXO1, REXO4, REXO5, RFC1, RFFL, RFK, RFLNB, RFNG, RFTN1,
RFWD3, RFX1, RFX2, RFX5, RFX7, RFXANK, RFXAP, RGCC, RGL1, RGL2, RGL3,
RGL4, RGN, RGP1, RGPD1, RGPD2, RGPD4, RGPD5, RGPD6, RGPD8, RGS1, RGS12,
RGS14, RGS16, RGS17, RGS18, RGS22, RGS3, RGS6, RGS7BP, RGS9, RHAG, RHBDD1,
RHBDF2, RHCE, RHCG, RHD, RHEB, RHNO1, RHO, RHOBTB2, RHOBTB3, RHOF,
RHOG, RHOQ, RHOT2, RHOU, RHOV, RHOXF2, RHOXF2B, RHPN1, RHPN2, RIBC1,
RIBC2, RIC1, RIC8A, RIDA, RIF1, RILP, RILPL2, RIMBP3, RIMBP3B, RIMKLB, RIMS1,
RIMS3, RIN1, RIN2, RINL, RIOK1, RIOK2, RIOK3, RIOX1, RIPK1, RIPOR1, RIPOR2,
RIPOR3, RMC1, RMDN3, RMND1, RMND5A, RMND5B, RNASE10, RNASE6, RNASE7,
RNASEH2C, RNASEK, RNASEL, RNASET2, RND3, RNF10, RNF11, RNF111, RNF121,
RNF122, RNF123, RNF125, RNF126, RNF13, RNF139, RNF144A, RNF145, RNF149,
RNF150, RNF152, RNF157, RNF165, RNF166, RNF167, RNF169, RNF180, RNF181,
RNF182, RNF187, RNF2, RNF20, RNF212, RNF215, RNF217, RNF220, RNF24, RNF25,
RNF26, RNF31, RNF38, RNF39, RNF40, RNF41, RNF44, RNF5, RNF6, RNF7, RNF8,
RNFT1, RNGTT, RNH1, RNLS, RNPC3, RNPEPL1, RNPS1, RO60, ROBO1, ROBO3,
ROCK2, ROGDI, ROMO1, ROPN1B, ROPN1L, RORB, ROS1, RP1, RPA1, RPA3, RPE,
RPF1, RPF2, RPGRIP1L, RPL11, RPL12, RPL13A, RPL14, RPL15, RPL17, RPL18A,
RPL19, RPL21, RPL22, RPL22L1, RPL23, RPL24, RPL26, RPL26L1, RPL27, RPL27A,
RPL28, RPL29, RPL30, RPL31, RPL35, RPL35A, RPL36, RPL36AL, RPL37, RPL37A,
RPL38, RPL41, RPL5, RPL6, RPL7, RPL7A, RPL7L1, RPL9, RPLP0, RPLP1, RPLP2,
RPN1, RPN2, RPP21, RPP25, RPP25L, RPP30, RPRD1A, RPRD2, RPS11, RPS12, RPS13,
RPS14, RPS15, RPS15A, RPS16, RPS17, RPS18, RPS19, RPS20, RPS24, RPS25, RPS27,
RPS27A, RPS27L, RPS29, RPS3A, RPS4X, RPS4Y1, RPS5, RPS6, RPS6KA1, RPS6KA2,
RPS6KA3, RPS6KA4, RPS6KB2, RPS7, RPS8, RPS9, RPSA, RPTN, RPUSD1, RPUSD4,
RRAGA, RRAGB, RRAGC, RRAGD, RRAS, RRBP1, RRM1, RRM2B, RRN3, RRP12,
RRP8, RRP9, RSAD2, RSBN1, RSF1, RSKR, RSL1D1, RSL24D1, RSPH1, RSPH10B,
RSPH10B2, RSPH3, RSPH4A, RSPH9, RSPO3, RSRC1, RSRC2, RSRP1, RTCA, RTEL1,
RTF2, RTL5, RTL6, RTL8A, RTL8B, RTL8C, RTN1, RTN3, RTP4, RTTN, RUBCN,
RUFY2, RUFY3, RUNDC1, RUNDC3A, RUNX3, RUSC2, RUVBL1, RWDD1, RWDD2B,
RWDD4, RXRA, RXRB, RXYLT1, RYK, RYR3, S100A10, S100A11, S100A12, S100A13,
S100A14, S100A16, S100A2, S100A3, S100A4, S100A6, S100A7, S100A9, S100B, S100P,
S100PBP, SIPR1, S1PR3, S1PR4, S1PR5, SAA1, SAE1, SAFB, SAFB2, SALL4, SAMD1,
SAMD10, SAMD12, SAMD14, SAMD15, SAMD3, SAMD4A, SAMD4B, SAMD8,
SAMD9, SAMD9L, SAMHD1, SAMM50, SAMSN1, SAP130, SAP18, SAP30, SAP30BP,
SAP30-DT, SAPCD2, SAR1B, SARAF, SARS1, SART1, SASH1, SASH3, SAV1, SAXO2,
SAYSD1, SBDS, SBF1, SBF2, SBF2-AS1, SBNO1, SBNO2, SCAF1, SCAF11, SCAF4,
SCAI, SCAMP2, SCAMP3, SCAMP4, SCAMP5, SCAND1, SCAP, SCARA3, SCARB2,
SCARF1, SCCPDH, SCD, SCD5, SCEL, SCFD2, SCGB1A1, SCGB3A1, SCGB3A2, SCIN,
SCLY, SCMH1, SCML4, SCN1B, SCN3A, SCNN1A, SCNN1D, SCNN1G, SCO1, SCOC,
SCP2, SCPEP1, SCRIB, SCRN2, SCRN3, SCUBE3, SCYL1, SDAD1, SDC2, SDC3,
SDCBP, SDCBP2, SDCCAG8, SDE2, SDF4, SDHAF2, SDHB, SDHC, SDHD, SDK1,
SDK2, SDR16C5, SDR39U1, SDS, SDSL, SEC11A, SEC14L1, SEC14L2, SEC14L5,
SEC14L6, SEC16A, SEC22A, SEC24A, SEC24B, SEC24C, SEC31A, SEC31B, SEC61A1,
SEC61A2, SEC61B, SEC61G, SECISBP2, SECISBP2L, SECTM1, SEH1L, SEL1L,
SELENBP1, SELENOI, SELENOK, SELENON, SELENOO, SELENOW, SELL, SELP,
SELPLG, SEM1, SEMA3A, SEMA3C, SEMA3F, SEMA4A, SEMA4B, SEMA4C,
SEMA4D, SEMA4F, SEMA6A, SEMA6B, SEMA6C, SEMA7A, SENP1, SENP3, SENP5,
SENP7, SENP8, SEPHS1, SEPHS2, SEPTIN1, SEPTIN10, SEPTIN11, SEPTIN3, SEPTIN4,
SEPTIN6, SEPTIN7, SEPTIN9, SERAC1, SERBP1, SERF1B, SERF2, SERGEF, SERHL2,
SERINC1, SERINC3, SERINC5, SERPINB1, SERPINB10, SERPINB11, SERPINB12,
SERPINB13, SERPINB2, SERPINB3, SERPINB4, SERPINB5, SERPINB9, SERPINE1,
SERPINE2, SERPINF1, SERPINF2, SERPING1, SERTAD2, SERTAD3, SERTAD4,
SESN2, SESN3, SETD1A, SETD1B, SETD2, SETD4, SETD6, SETD7, SETDB1, SETDB2,
SETMAR, SETX, SF1, SF3A1, SF3A2, SF3A3, SF3B1, SF3B2, SF3B3, SF3B4, SF3B6,
SFI1, SFMBT2, SFPQ, SFR1, SFRP5, SFSWAP, SFT2D2, SFTPA1, SFTPA2, SFTPB,
SFTPC, SFTPD, SFXN1, SFXN5, SGCE, SGK1, SGO2, SGPL1, SGPP2, SGSH, SGSM2,
SGSM3, SGTA, SH2B1, SH2B2, SH2B3, SH2D1A, SH2D1B, SH2D2A, SH2D3A,
SH2D3C, SH3BGRL, SH3BGRL3, SH3BP1, SH3BP2, SH3BP4, SH3BP5L, SH3D19,
SH3GL1, SH3GLB2, SH3KBP1, SH3PXD2A, SH3TC1, SH3TC2, SHARPIN, SHC1, SHC3,
SHC4, SHCBP1, SHFL, SHISA2, SHISA5, SHISA6, SHISA7, SHISAL2A, SHKBP1,
SHLD3, SHMT1, SHMT2, SHOC1, SHOC2, SHQ1, SHROOM2, SHROOM3, SHTN1,
SIAE, SIAH1, SIAH2, SIAH3, SIDT1, SIGIRR, SIGLEC11, SIGLEC12, SIGLEC16,
SIGLEC9, SIGMAR1, SIK1, SIK1B, SIK2, SIK3, SIN3B, SIPA1, SIPA1L1, SIPA1L2,
SIPA1L3, SIRPA, SIRPB2, SIRPD, SIRT2, SIRT3, SIRT5, SIRT7, SITI, SIVA1, SIX1,
SIX2, SIX4, SKA2, SKA3, SKAP1, SKI, SKIL, SKIV2L, SKP1, SLA, SLA2, SLAIN1,
SLAIN2, SLAMF8, SLAMF9, SLBP, SLC10A3, SLC10A5, SLC11A1, SLC11A2,
SLC12A1, SLC12A4, SLC12A6, SLC12A7, SLC12A9, SLC14A1, SLC15A2, SLC15A3,
SLC15A4, SLC16A10, SLC16A13, SLC16A3, SLC16A5, SLC16A6, SLC16A7, SLC16A8,
SLC16A9, SLC17A9, SLC18B1, SLC19A1, SLC19A2, SLC19A3, SLC1A1, SLC1A3,
SLC1A5, SLC20A1, SLC22A13, SLC22A15, SLC22A17, SLC22A18, SLC22A3, SLC23A1,
SLC23A2, SLC24A3, SLC24A4, SLC25A1, SLC25A11, SLC25A14, SLC25A15,
SLC25A17, SLC25A20, SLC25A22, SLC25A23, SLC25A24, SLC25A28, SLC25A29,
SLC25A3, SLC25A30, SLC25A35, SLC25A36, SLC25A37, SLC25A38, SLC25A39,
SLC25A4, SLC25A42, SLC25A43, SLC25A45, SLC25A6, SLC26A1, SLC26A6, SLC26A8,
SLC27A1, SLC27A2, SLC27A3, SLC29A1, SLC29A2, SLC29A3, SLC2A1, SLC2A10,
SLC2A11, SLC2A12, SLC2A14, SLC2A3, SLC2A4RG, SLC2A6, SLC2A9, SLC30A6,
SLC30A7, SLC30A9, SLC31A1, SLC31A2, SLC33A1, SLC34A2, SLC35A2, SLC35A3,
SLC35A4, SLC35B2, SLC35B4, SLC35C1, SLC35C2, SLC35D1, SLC35E2A, SLC35E2B,
SLC35E4, SLC35F2, SLC35F3, SLC35F6, SLC37A1, SLC37A2, SLC37A3, SLC37A4,
SLC38A1, SLC38A10, SLC38A2, SLC38A5, SLC38A6, SLC38A7, SLC38A9, SLC39A1,
SLC39A13, SLC39A14, SLC39A3, SLC39A7, SLC3A2, SLC41A2, SLC43A1, SLC43A2,
SLC44A1, SLC44A2, SLC44A3, SLC44A4, SLC46A2, SLC47A1, SLC49A3, SLC49A4,
SLC4A1, SLC4A11, SLC4A1AP, SLC4A2, SLC4A4, SLC4A7, SLC51A, SLC52A2,
SLC52A3, SLC5A4, SLC5A6, SLC5A9, SLC66A1, SLC66A2, SLC66A3, SLC6A12,
SLC6A14, SLC6A16, SLC6A20, SLC6A6, SLC6A8, SLC6A9, SLC7A1, SLC7A11,
SLC7A2, SLC7A5, SLC7A6, SLC7A7, SLC7A8, SLC8A1, SLC8A3, SLC8B1, SLC9A3R2,
SLC9A6, SLC9A7, SLC9A8, SLC9B2, SLC9C2, SLCO2A1, SLCO2B1, SLCO3A1,
SLCO4C1, SLF2, SLFN13, SLFN14, SLITRK4, SLITRK6, SLK, SLMAP, SLPI, SLX1A,
SLX1B, SLX4, SLX4IP, SMAD3, SMAD5, SMAD9, SMAP2, SMARCA1, SMARCA2,
SMARCA4, SMARCA5, SMARCAD1, SMARCB1, SMARCC1, SMARCD1, SMARCD2,
SMARCD3, SMC1A, SMC2, SMC6, SMCHD1, SMCR8, SMG1, SMG1P7, SMG5, SMG9,
SMIM11A, SMIM11B, SMIM12, SMIM14, SMIM15, SMIM19, SMIM20, SMIM22,
SMIM24, SMIM25, SMIM26, SMIM29, SMIM3, SMIM30, SMIM31, SMIM4, SMIM5,
SMIM6, SMIM7, SMIM8, SMKR1, SMNDC1, SMOC2, SMOX, SMPD1, SMPD2, SMPD3,
SMPD4, SMPDL3A, SMR3B, SMS, SMTN, SMTNL1, SMYD2, SMYD4, SMYD5, SNAI1,
SNAI2, SNAP23, SNAP47, SNAPC3, SNAPC4, SNAPC5, SNAPIN, SNCA, SNCAIP,
SND1, SNHG1, SNHG16, SNHG3, SNIP1, SNN, SNPH, SNRK, SNRNP200, SNRNP25,
SNRNP27, SNRNP35, SNRNP70, SNRPA, SNRPA1, SNRPB, SNRPB2, SNRPD1, SNRPG,
SNTB1, SNTN, SNU13, SNUPN, SNW1, SNX1, SNX10, SNX11, SNX13, SNX17, SNX18,
SNX19, SNX2, SNX20, SNX22, SNX24, SNX27, SNX30, SNX33, SNX5, SNX6, SNX7,
SNX9, SOAT1, SOBP, SOCS3, SOCS5, SOCS6, SOCS7, SOD1, SON, SORBS2, SORBS3,
SORCS2, SORD, SORL1, SOS1, SOSTDC1, SOWAHC, SOX12, SOX2, SOX21, SOX5,
SOX6, SOX9, SP110, SP2, SPA17, SPACA9, SPAG1, SPAG16, SPAG17, SPAG6, SPAG7,
SPAG8, SPAG9, SPARCL1, SPART, SPAST, SPATA17, SPATA18, SPATA2, SPATA20,
SPATA2L, SPATA5, SPATC1L, SPATS2, SPATS2L, SPCS3, SPDEF, SPDL1, SPDYE2,
SPDYE2B, SPDYE3, SPDYE6, SPECC1L, SPEF1, SPEF2, SPEG, SPEN, SPG21, SPG7,
SPHK1, SPHK2, SPI1, SPIB, SPIC, SPIDR, SPIN1, SPIN2A, SPIN2B, SPIN4, SPINDOC,
SPINK5, SPINK7, SPINT2, SPIRE1, SPN, SPNS1, SPNS2, SPNS3, SPOCK2, SPON2,
SPOP, SPOUT1, SPP1, SPPL2B, SPPL3, SPRED1, SPRED2, SPRR1A, SPRR1B, SPRR2A,
SPRR2B, SPRR2D, SPRR2E, SPRR2F, SPRR2G, SPRR3, SPRY4, SPRYD3, SPRYD7,
SPSB2, SPTA1, SPTB, SPTBN1, SPTBN2, SPTBN5, SPTLC1, SPTLC3, SQLE, SQOR,
SQSTM1, SRA1, SRBD1, SRCAP, SRD5A1, SRD5A2, SRD5A3, SREBF1, SREBF2,
SREK1, SREK1IP1, SRF, SRFBP1, SRGAP2, SRGAP2B, SRGAP2C, SRGAP3, SRI, SRM,
SRP14, SRP72, SRPRA, SRPRB, SRR, SRRM1, SRRM2, SRRM4, SRRT, SRSF1, SRSF10,
SRSF11, SRSF2, SRSF3, SRSF4, SRSF5, SRSF6, SRSF7, SRSF9, SS18L1, SS18L2, SSB,
SSBP1, SSBP3, SSBP4, SSH1, SSNA1, SSPN, SSR2, SSR3, SSR4, SSRP1, SSTR2, SSTR3,
SSX2IP, STB, STM, ST3GAL1, ST3GAL2, ST3GAL6, ST6GAL1, ST6GALNAC1,
ST6GALNAC3, ST6GALNAC4, ST7, STAB1, STAC, STAC3, STAG1, STAG3, STAM,
STAM2, STAP1, STAP2, STARD13, STARD3, STARD3NL, STARD4, STARD5,
STARD7, STARD9, STAT1, STAT3, STAT4, STAT5A, STAT5B, STAT6, STATH,
STAU1, STAU2, STEAP1, STEAP1B, STEAP2, STIL, STIM1, STIMATE, STIMATE-
MUSTN1, STING1, STIP1, STK10, STK11, STK11IP, STK16, STK17A, STK17B, STK24,
STK25, STK26, STK3, STK32C, STK33, STK35, STK38, STK39, STK4, STK40, STMN1,
STMN3, STMND1, STMP1, STN1, STOML1, STON2, STOX1, STOX2, STPG1, STRADA,
STRADB, STRAP, STRBP, STRIP1, STRN3, STRN4, STT3A, STT3B, STUB1, STX10,
STX12, STX16, STX17, STX18, STX19, STX1A, STX2, STX5, STX6, STX7, STX8,
STXBP1, STXBP2, STXBP4, STXBP6, STYX, SUB1, SUCNR1, SUCO, SUDS3, SUFU,
SUGP1, SUGP2, SUGT1, SULF1, SULF2, SULT1A2, SULT1A3, SULT1A4, SULT1B1,
SULT1C2, SULT2B1, SUMF1, SUMF2, SUMO2, SUN1, SUN2, SUOX, SUPT16H,
SUPT5H, SUPV3L1, SURF1, SURF4, SURF6, SUSD3, SUSD6, SUV39H1, SUV39H2,
SV2A, SVEP1, SWAP70, SYBU, SYCP2, SYCP3, SYK, SYMPK, SYN1, SYNDIG1,
SYNE1, SYNE2, SYNE3, SYNGAP1, SYNGR1, SYNGR2, SYNJ1, SYNJ2BP, SYNM,
SYNPO, SYNPO2, SYNRG, SYS1, SYT11, SYT17, SYT6, SYTL1, SYTL2, SYTL3,
SYTL5, SYVN1, SZRD1, SZT2, TAB1, TAB2, TACC2, TACC3, TACSTD2, TADA1,
TADA2A, TADA3, TAF10, TAF13, TAF15, TAF1C, TAF3, TAF6, TAF6L, TAF8, TAF9,
TAF9B, TAGAP, TAGLN2, TAL1, TALDO1, TANC1, TANC2, TANGO2, TANK, TAOK1,
TAOK2, TAPI1, TAP2, TAPBP, TAPT1, TARBP2, TARDBP, TARS1, TAS1R3, TAS2RB,
TAS2R30, TASOR, TATDN2, TAX1BP1, TAX1BP3, TAZ, TBC1D10B, TBC1D10C,
TBC1D14, TBC1D15, TBC1D17, TBC1D22A, TBC1D23, TBC1D24, TBC1D25,
TBC1D2B, TBC1D3, TBC1D3C, TBC1D3D, TBC1D3E, TBC1D3H, TBC1D3I, TBC1D3K,
TBC1D5, TBC1D8B, TBC1D9B, TBCC, TBCD, TBCE, TBCEL, TBKBP1, TBL1X, TBL3,
TBP, TBPL1, TBRG1, TBRG4, TBX1, TBX19, TBX20, TBX21, TBXA2R, TBXAS1,
TCAF1, TCAF2, TCAIM, TCEA1, TCEA2, TCEA3, TCEAL1, TCEAL3, TCEAL4,
TCEAL8, TCEAL9, TCEANC2, TCF12, TCF25, TCF3, TCF7, TCF7L1, TCF7L2, TCHH,
TCHP, TCIRG1, TCL1A, TCTA, TCTE1, TCTEX1D1, TCTN1, TCTN2, TDG, TDP1,
TDP2, TDRD3, TDRD6, TDRD7, TDRD9, TEAD1, TEAD3, TECPR1, TECR, TEDC1,
TEKT1, TEKT2, TEKT4, TELO2, TENM1, TENM4, TENT2, TENT4A, TENT5A,
TENT5C, TEP1, TEPSIN, TERF1, TERF2IP, TES, TESC, TESK1, TESK2, TESMIN,
TESPA1, TET1, TET2, TET3, TEXM, TEX26, TEX261, TEX264, TEX9, TFAP2A,
TFAP2E, TFAP4, TFB2M, TFCP2L1, TFDP2, TFE3, TFEB, TFEC, TFF1, TFF2, TFF3,
TFPI, TFR2, TFRC, TG, TGFB1, TGFB2, TGFB3, TGFBI, TGFBR3, TGFBRAP1, TGM3,
TGOLN2, TGS1, THADA, THAP10, THAP5, THAP6, THAP8, THBS1, THEGL, THEMIS,
THEMIS2, THOC3, THOC5, THOC6, THOP1, THRA, THRAP3, THRB, THSD1, THTPA,
THUMPD3, THYN1, TIAF1, TIAL1, TIAM2, TICAM1, TIFA, TIGD1, TIGD5, TIGD6,
TIGIT, TIMM13, TIMM17B, TIMM21, TIMM22, TIMM23, TIMM23B, TIMM44,
TIMM50, TIMM8A, TIMM8B, TIMM9, TIMMDC1, TINAGL1, TINF2, TIPARP, TIPRL,
TIRAP, TJAP1, TJP1, TKFC, TKT, TKTL1, TLCD2, TLCD3A, TLCD5, TLE1, TLE3,
TLE5, TLK2, TLL1, TLN1, TLN2, TLR10, TLR2, TLR3, TLR4, TLR7, TLR8, TM2D2,
TM2D3, TM4SF1, TM7SF2, TM7SF3, TM9SF2, TM9SF3, TM9SF4, TMBIM1, TMBIM4,
TMBIM6, TMC5, TMC6, TMC8, TMCC1, TMCC2, TMCO1, TMCO3, TMCO5A, TMCO6,
TMED1, TMED3, TMED4, TMED6, TMED8, TMED9, TMEM102, TMEM104,
TMEM106A, TMEM106B, TMEM106C, TMEM109, TMEM11, TMEM115, TMEM120A,
TMEM120B, TMEM121B, TMEM126A, TMEM126B, TMEM127, TMEM129,
TMEM131L, TMEM132D, TMEM138, TMEM139, TMEM143, TMEM14A, TMEM14C,
TMEM150B, TMEM154, TMEM156, TMEM159, TMEM160, TMEM161A, TMEM161B,
TMEM164, TMEM167A, TMEM167B, TMEM168, TMEM170A, TMEM170B, TMEM175,
TMEM176B, TMEM179B, TMEM181, TMEM183A, TMEM184B, TMEM184C,
TMEM185A, TMEM186, TMEM191C, TMEM199, TMEM201, TMEM204, TMEM205,
TMEM208, TMEM209, TMEM212, TMEM214, TMEM216, TMEM217, TMEM222,
TMEM223, TMEM229B, TMEM232, TMEM237, TMEM241, TMEM242, TMEM248,
TMEM250, TMEM251, TMEM254, TMEM255A, TMEM259, TMEM263, TMEM266,
TMEM272, TMEM273, TMEM30B, TMEM33, TMEM38A, TMEM38B, TMEM39B,
TMEM40, TMEM41A, TMEM43, TMEM44, TMEM50A, TMEM50B, TMEM51,
TMEM52B, TMEM53, TMEM54, TMEM59, TMEM60, TMEM63A, TMEM63B, TMEM67,
TMEM68, TMEM69, TMEM71, TMEM79, TMEM80, TMEM86A, TMEM86B, TMEM87A,
TMEM87B, TMEM88, TMEM8B, TMEM94, TMEM97, TMIGD2, TMOD1, TMOD3,
TMPO, TMPPE, TMPRSS11A, TMPRSS11B, TMPRSS11D, TMPRSS11E, TMPRSS2,
TMPRSS4, TMPRSS9, TMSB10, TMSB4X, TMTC2, TMTC3, TMTC4, TMUB1, TMUB2,
TMX2, TMX4, TNC, TNFAIP1, TNFAIP2, TNFAIP3, TNFAIP6, TNFAIP8, TNFAIP8L1,
TNFAIP8L2, TNFRSF10A, TNFRSF10B, TNFRSF10C, TNFRSF10D, TNFRSF11A,
TNFRSF12A, TNFRSF13B, TNFRSF13C, TNFRSF14, TNFRSF17, TNFRSF19,
TNFRSF1B, TNFRSF21, TNFRSF25, TNFRSF4, TNFRSF8, TNFSF10, TNFSF11,
TNFSF12, TNFSF13, TNFSF13B, TNFSF15, TNFSF4, TNFSF8, TNFSF9, TNIP1, TNIP2,
TNIP3, TNK2, TNNI2, TNPO1, TNRC18, TNRC6B, TNS3, TNS4, TNXB, TOB1, TOB2,
TOGARAM2, TOLLIP, TOM1, TOM1L1, TOM1L2, TOMM20, TOMM34, TOMM40L,
TOMM5, TOMM7, TONSL, TOP1MT, TOP3A, TOPORS, TOR1B, TOR2A, TOR3A,
TOR4A, TOX2, TOX3, TP53BP1, TP53I13, TP53I3, TP53INP1, TP53RK, TP63, TPCN1,
TPCN2, TPD52, TPD52L1, TPD52L2, TPGS1, TPI1, TPM2, TPM4, TPP2, TPPP3, TPR,
TPRG1, TPRN, TPSB2, TPST2, TPT1, TRA2A, TRA2B, TRABD, TRABD2A, TRADD,
TRAF1, TRAF2, TRAF3IP1, TRAF3IP3, TRAF4, TRAF6, TRAF7, TRAM2, TRANK1,
TRAP1, TRAPPC1, TRAPPC10, TRAPPC13, TRAPPC2B, TRAPPC4, TRAPPC5,
TRAPPC6B, TRAPPC9, TRAT1, TREM2, TREML1, TREML2, TRERF1, TRGC1, TRHDE,
TRIAPI, TRIB1, TRIB2, TRIB3, TRIM10, TRIM11, TRIM13, TRIM14, TRIM16, TRIM2,
TRIM21, TRIM23, TRIM24, TRIM28, TRIM29, TRIM32, TRIM33, TRIM35, TRIM36,
TRIM39, TRIM41, TRIM44, TRIM46, TRIM47, TRIM5, TRIM52, TRIM56, TRIM58,
TRIM6, TRIM62, TRIM65, TRIM66, TRIM68, TRIM69, TRIM71, TRIM8, TRIO, TRIOBP,
TRIP10, TRIP11, TRIP13, TRIP6, TRIQK, TRIR, TRMT1, TRMT10C, TRMT11, TRMT1L,
TRMT2A, TRMT2B, TRMT5, TRMT6, TRMT61A, TRMT61B, TRMU, TRNAU1AP,
TROAP, TRPC4AP, TRPC6, TRPM2, TRPM3, TRPV2, TRPV4, TRUB2, TSACC, TSC1,
TSC2, TSC22D2, TSC22D3, TSC22D4, TSEN2, TSEN34, TSEN54, TSFM, TSG101,
TSGA10, TSHZ2, TSNARE1, TSNAX, TSPAN1, TSPAN11, TSPAN12, TSPAN14,
TSPAN15, TSPAN17, TSPAN18, TSPAN19, TSPAN2, TSPAN3, TSPAN31, TSPAN32,
TSPAN33, TSPAN5, TSPAN6, TSPAN8, TSPAN9, TSPO, TSPO2, TSPYL2, TSSC4,
TSSK4, TST, TSTA3, TSTD1, TSTD2, TTBK2, TTC1, TTC13, TTC14, TTC16, TTC17,
TTC21A, TTC22, TTC23, TTC24, TTC25, TTC26, TTC27, TTC29, TTC3, TTC30A,
TTC30B, TTC31, TTC32, TTC34, TTC37, TTC38, TTC39A, TTC5, TTC6, TTC7A, TTC7B,
TTC8, TTC9, TTF1, TTI1, TTL, TTLL1, TTLL11, TTLL12, TTLL3, TTLL5, TTLL6,
TTLL7, TTN, TTYH1, TTYH2, TTYH3, TUBA1C, TUBA4A, TUBA4B, TUBB, TUBB1,
TUBB2A, TUBB4B, TUBB6, TUBE1, TUBG1, TUBGCP2, TUBGCP3, TUBGCP6, TUFM,
TULP3, TULP4, TUSC1, TUSC2, TUSC3, TUSC7, TUT7, TWF2, TWISTNB, TWNK,
TWSG1, TXK, TXLNB, TXLNG, TXN, TXN2, TXNDC11, TXNDC12, TXNDC15,
TXNDC17, TXNDC5, TXNDC9, TXNRD1, TXNRD2, TXNRD3, TYK2, TYMP, TYROBP,
TYW1, TYWIB, TYW3, U2AF1, U2AF1L4, U2AF1L5, U2AF2, UACA, UAP1, UAP1L1,
UBA1, UBA2, UBA6, UBA7, UBAC2, UBALD1, UBALD2, UBAP1L, UBAP2, UBAP2L,
UBASH3A, UBASH3B, UBB, UBC, UBE2C, UBE2D3, UBE2D4, UBE2E1, UBE2F,
UBE2G1, UBE2G2, UBE2H, UBE2J2, UBE2L3, UBE2M, UBE2N, UBE2O, UBE2Q1,
UBE2Q2, UBE2S, UBE2V1, UBE2V2, UBE2W, UBE2Z, UBE3A, UBE3C, UBE3D,
UBE4A, UBFD1, UBIAD1, UBL3, UBL5, UBL7, UBN1, UBP1, UBQLNL, UBR1, UBR3,
UBR4, UBR5, UBR7, UBTD1, UBTD2, UBTF, UBXN1, UBXN10, UBXN11, UBXN2B,
UBXN6, UBXN7, UBXN8, UCHL1, UCK1, UCKL1, UCP1, UCP2, UEVLD, UFD1,
UGDH, UGGT1, UGGT2, UGP2, UGT2A1, UGT2B28, UGT3A2, UHRF1, UHRF1BP1,
UHRF1BP1L, UHRF2, ULBP2, ULK1, ULK4, UMAD1, UNC119, UNC119B, UNC13B,
UNC13D, UNC45A, UNC50, UNC5CL, UNC93B1, UNK, UNKL, UPB1, UPF1, UPF3A,
UPK1B, UPK3B, UPK3BL2, UPP1, UQCC2, UQCR10, UQCR11, UQCRB, UQCRC1,
UQCRH, UQCRQ, URB2, URM1, UROD, USF1, USF2, USF3, USO1, USP1, USP11,
USP14, USP15, USP19, USP20, USP21, USP22, USP25, USP31, USP32, USP35, USP38,
USP39, USP46, USP47, USP48, USP5, USP51, USP53, USP54, USP6NL, USP7, USP9X,
UST, UTP14A, UTP15, UTP18, UTP20, UTP25, UTP3, UTP4, UTP6, UTRN, UVSSA,
VAC14, VAMP1, VAMP2, VAMP3, VAMP4, VAMP8, VANGL2, VARS1, VARS2, VASP,
VAT1, VAV1, VAV2, VCAN, VCL, VCP, VCPIP1, VCPKMT, VDAC1, VDAC2, VEGFA,
VEGFB, VENTX, VEPH1, VEZF1, VEZT, VGLL1, VGLL3, VHL, VIL1, VILL, VIPR1,
VKORC1, VKORC1L1, VLDLR, VMAC, VMO1, VMP1, VNN1, VNN2, VNN3, VPREB3,
VPS13A, VPS13D, VPS16, VPS18, VPS26A, VPS26B, VPS28, VPS29, VPS33B, VPS35,
VPS37A, VPS37B, VPS39, VPS41, VPS4A, VPS4B, VPS50, VPS51, VPS52, VPS72,
VPS9D1, VRK1, VRK2, VRK3, VSIG1, VSIG10, VSIG10L, VSIG2, VSIR, VSNL1,
VSTM1, VSTM2L, VTA1, VTCN1, VTI1A, VWA3A, VWA3B, VWA5A, VWA8, VWF,
WAC, WAPL, WAS, WASF1, WASF2, WASHC1, WASHC4, WASHC5, WASL, WBP1,
WBP11, WBP2, WBP4, WDCP, WDFY1, WDFY2, WDFY4, WDHD1, WDR1, WDR11,
WDR13, WDR18, WDR19, WDR20, WDR24, WDR33, WDR34, WDR36, WDR37,
WDR38, WDR4, WDR43, WDR44, WDR45, WDR45B, WDR46, WDR49, WDR5, WDR55,
WDR59, WDR6, WDR60, WDR61, WDR62, WDR63, WDR66, WDR7, WDR70, WDR73,
WDR74, WDR76, WDR78, WDR81, WDR82, WDR83, WDR830S, WDR86, WDR89,
WDR90, WDR91, WDR92, WDR93, WDR97, WDTC1, WDYHV1, WFDC1, WFDC2,
WFDC3, WHAMM, WHRN, WIPF2, WIPI1 WIPI2, WIZ, WNK2, WNK3, WNT10B,
WNT11, WNT2B, WNT4, WNT5A, WRAP53, WRAP73, WRN, WRNIP1, WSB2, WWC1,
WWC2, WWC3, WWP2, WWTR1, XAB2, XAF1, XG, XIAP, XK, XKR6, XKR8, XPA,
XPC, XPNPEP1, XPNPEP3, XPO1, XPO4, XPO6, XPO7, XPOT, XRCC3, XRCC4, XRN1,
XRN2, XRRA1, XXYLT1, XYLT1, XYLT2, YAE1, YAP1, YARS1, YARS2, YBX3,
YEATS4, YES1, YIF1A, YIF1B, YIPF2, YIPF3, YIPF4, YIPF5, YIPF6, YJU2, YKT6,
YLPM1, YME1L1, YOD1, YPEL1, YPEL2, YPEL3, YPEL4, YPEL5, YRDC, YTHDC1,
YTHDF1, YTHDF2, YTHDF3, YWHAB, YWHAE, YWHAG, YWHAQ, YY1, ZADH2,
ZAP70, ZBBX, ZBED3, ZBED4, ZBED5, ZBED6CL, ZBED8, ZBP1, ZBTB1, ZBTB10,
ZBTB11, ZBTB16, ZBTB17, ZBTB18, ZBTB2, ZBTB21, ZBTB22, ZBTB24, ZBTB25,
ZBTB32, ZBTB34, ZBTB37, ZBTB38, ZBTB39, ZBTB40, ZBTB41, ZBTB43, ZBTB46,
ZBTB47, ZBTB48, ZBTB5, ZBTB6, ZBTB7B, ZBTB7C, ZBTB8OS, ZC3H11B, ZC3H12A,
ZC3H13, ZC3H14, ZC3H15, ZC3H3, ZC3H4, ZC3H6, ZC3H7B, ZC3HAV1L, ZCCHC10,
ZCCHC17, ZCCHC3, ZCCHC7, ZCCHC9, ZCRB1, ZDHHC11, ZDHHC12, ZDHHC16,
ZDHHC17, ZDHHC18, ZDHHC20, ZDHHC21, ZDHHC24, ZDHHC5, ZDHHC6, ZDHHC7,
ZDHHC8, ZEB2, ZER1, ZFAND1, ZFAND2A, ZFAND2B, ZFAND3, ZFAND5, ZFHX2,
ZFHX3, ZFP1, ZFP14, ZFP3, ZFP36, ZFP36L2, ZFP41, ZFP69B, ZFP91, ZFPL1, ZFPM1,
ZFR, ZFX, ZFYVE1, ZFYVE16, ZFYVE19, ZFYVE21, ZFYVE26, ZFYVE27, ZFYVE28,
ZFYVE9, ZG16B, ZGPAT, ZGRF1, ZHX1, ZKSCAN2, ZKSCAN5, ZKSCAN8, ZMAT1,
ZMAT5, ZMIZ1, ZMIZ2, ZMYM3, ZMYM4, ZMYM6, ZMYND10, ZMYND11,
ZMYND12, ZMYND19, ZMYND8, ZNF101, ZNF107, ZNF12, ZNF121, ZNF124, ZNF134,
ZNF135, ZNF138, ZNF14, ZNF141, ZNF142, ZNF148, ZNF16, ZNF160, ZNF169, ZNF175,
ZNF181, ZNF184, ZNF189, ZNF19, ZNF200, ZNF202, ZNF211, ZNF212, ZNF213,
ZNF214, ZNF217, ZNF219, ZNF22, ZNF222, ZNF224, ZNF226, ZNF230, ZNF232,
ZNF234, ZNF235, ZNF251, ZNF253, ZNF26, ZNF260, ZNF268, ZNF273, ZNF276,
ZNF277, ZNF28, ZNF280B, ZNF280D, ZNF281, ZNF282, ZNF283, ZNF285, ZNF287,
ZNF296, ZNF3, ZNF300, ZNF302, ZNF316, ZNF317, ZNF318, ZNF319, ZNF324,
ZNF324B, ZNF330, ZNF331, ZNF333, ZNF335, ZNF337, ZNF33B, ZNF34, ZNF341,
ZNF343, ZNF346, ZNF347, ZNF35, ZNF350, ZNF354B, ZNF354C, ZNF358, ZNF362,
ZNF365, ZNF367, ZNF37A, ZNF382, ZNF385A, ZNF394, ZNF395, ZNF407, ZNF408,
ZNF41, ZNF414, ZNF418, ZNF419, ZNF420, ZNF426, ZNF428, ZNF429, ZNF43, ZNF430,
ZNF431, ZNF436, ZNF438, ZNF439, ZNF440, ZNF441, ZNF442, ZNF444, ZNF445,
ZNF449, ZNF451, ZNF454, ZNF462, ZNF467, ZNF468, ZNF469, ZNF470, ZNF471,
ZNF473, ZNF474, ZNF48, ZNF480, ZNF484, ZNF486, ZNF493, ZNF500, ZNF501,
ZNF510, ZNF512, ZNF512B, ZNF513, ZNF514, ZNF516, ZNF518A, ZNF518B, ZNF524,
ZNF525, ZNF527, ZNF528, ZNF532, ZNF540, ZNF547, ZNF548, ZNF552, ZNF554,
ZNF555, ZNF557, ZNF561, ZNF562, ZNF564, ZNF566, ZNF569, ZNF570, ZNF572,
ZNF576, ZNF579, ZNF580, ZNF585B, ZNF586, ZNF587B, ZNF592, ZNF595, ZNF597,
ZNF598, ZNF599, ZNF605, ZNF606, ZNF607, ZNF610, ZNF611, ZNF613, ZNF614,
ZNF616, ZNF619, ZNF621, ZNF622, ZNF624, ZNF626, ZNF628, ZNF638, ZNF641,
ZNF644, ZNF646, ZNF649, ZNF654, ZNF658, ZNF66, ZNF665, ZNF668, ZNF675,
ZNF678, ZNF680, ZNF687, ZNF69, ZNF691, ZNF692, ZNF697, ZNF699, ZNF7, ZNF701,
ZNF704, ZNF706, ZNF707, ZNF708, ZNF71, ZNF710, ZNF713, ZNF721, ZNF727,
ZNF737, ZNF738, ZNF740, ZNF746, ZNF749, ZNF750, ZNF75D, ZNF76, ZNF761,
ZNF764, ZNF765, ZNF766, ZNF768, ZNF770, ZNF771, ZNF774, ZNF780B, ZNF782,
ZNF784, ZNF785, ZNF787, ZNF789, ZNF79, ZNF790, ZNF791, ZNF799, ZNF808, ZNF81,
ZNF813, ZNF816, ZNF821, ZNF823, ZNF829, ZNF83, ZNF831, ZNF835, ZNF836,
ZNF839, ZNF84, ZNF844, ZNF845, ZNF85, ZNF850, ZNF852, ZNF860, ZNF862, ZNF865,
ZNF880, ZNF888, ZNF891, ZNF90, ZNF91, ZNF92, ZNF93, ZNHIT3, ZNHIT6, ZP3,
ZPR1, ZRANB1, ZSCAN12, ZSCAN2, ZSCAN25, ZSCAN31, ZSCAN9, ZSWIMI,
ZSWIM4, ZSWIM7, ZSWIM8, ZSWIM9, ZW10, ZWILCH, ZWINT, ZXDB, ZXDC, ZYX,
ZZEF1

TABLE 7A
(Gene lists defining myeloid populations increased in 3 compartments
derived from co-expression analyses)
CoV-2 PBMC Myeloid A1
ADAMDEC1, ADGRE2, AIF1, AOAH, APOBEC3B, APOBR, APOC1, BST1, C1QA,
C1QB, C1QC, C1RL, C2, C4A, C4B, C4BPA, C5, C6, CCL18, CCL2, CCL7, CCL8, CD101,
CD14, CD163, CD209, CD300C, CD300E, CD300LF, CD33, CD68, CD80, CFD, CFP,
CLEC10A, CLEC12A, CLEC12B, CLEC16A, CLEC4A, CLEC4D, CLEC4E, CLEC5A,
CLEC6A, CLEC7A, CSF1R, CSF2RA, CSF2RB, CSF3R, CST3, CXCL10, CXCL11,
CXCL2, CXCL9, CYBA, CYBB, FCER1G, FCGR1A, FCGR1B, FCGR2A, FCGR2B,
FCGR2C, FGR, FLT3, FOLR2, FUT4, GRN, HVCN1, IFI30, IGSF6, IL10RA, IL18, IL1A,
IL1RAP, IL1RN, ITGAX, LGALS12, LGALS9, LILRA1, LILRA2, LILRA5, LILRA6,
LILRB1, LILRB2, LILRB3, LILRB4, LILRB5, LMNB1, LY86, LYVE1, LYZ, MARCO,
MEFV, MERTK, MFGE8, MNDA, MPEG1, MRC1, MS4A4A, MS4A6A, MSR1, NLRP12,
NLRP3, NOD2, NTSR1, OSCAR, OTOF, PECAM1, PILRA, PLEK, PRAM1, RNASE3,
RNASE6, RNASE7, S100A8, S100A9, SCARB1, SECTM1, SEMA4A, SERPINB9,
SERPING1, SIGLEC1, SIGLEC10, SIGLEC5, SIGLEC7, SKAP2, SLC11A1, SLITRK4,
SPI1, TGM2, THBD, TLR2, TLR8, TNF, TNFAIP8L2, TNFRSF1B, TNFSF13B, TREM1,
TREML4, TYROBP, UNC93B1, VENTX, VSIG4, VSTM1
CoV-2 Lung Myeloid A2
ADAMDEC1, ADGRE1, ADGRE2, AIF1, APOBEC3B, APOBEC3G, APOBR, APOC1,
BST1, C1QA, C1QB, C1QC, C2, C4BPA, C4BPB, C6, C8A, CCL18, CCL2, CCL22, CCL7,
CCL8, CD14, CD300E, CD33, CD80, CFD, CFP, CHI3L1, CHIT1, CLEC12A, CLEC12B,
CLEC1A, CLEC2B, CLEC4A, CLEC4D, CLEC4E, CLEC7A, CSF2, CSF2RB, CSF3R,
CST3, CXCL1, CXCL10, CXCL11, CXCL13, CXCL8, CXCR2, CYBA, CYBB, FCER1G,
FCGR1A, FCGR2A, FCGR2B, FCGR2C, FCGR3A, FCGR3B, FFAR2, FGR, FOLR2, GRN,
IFNL1, IGSF6, IL12B, IL18, IL18RAP, IL1A, IL1B, IL1RAP, IL1RN, IL20, IL23A, IL27,
ITGAX, LAMP3, LGALS9, LGALS9C, LILRA5, LILRA6, LILRB1, LILRB2, LILRB3,
LILRB4, LILRB5, LMNB1, LY86, LYZ, MNDA, MPEG1, MS4A4A, MS4A6A, MSR1,
NLRP3, OLR1, OTOF, PDCD1LG2, PILRA, PLEK, S100A8, S100A9, S1PR5, SECTM1,
SEMA4A, SERPINB9, SERPING1, SIGLEC14, SIGLEC5, SLPI, SPI1, TLR2, TNF,
TNFSF13B, TNFSF14, TNIP3, TREM1, TREML4, TYROBP, UNC93B1, VSIG4
CoV-2 BAL Myeloid A3
ACE, ADAMDEC1, ADGRE3, AIF1, APOBEC3G, APOC1, C1QA, C1QB, C1QC, C1R,
C2, C4BPA, C5, C6, CCL18, CCL2, CCL28, CCL7, CCL8, CD163, CD300LF, CD5L,
CD68, CD80, CHI3L1, CLEC2B, CLEC4E, CLEC5A, CLEC6A, CXCL1, CXCL10,
CXCL11, CXCL2, CXCL8, CXCL9, CXCR2, FCER1G, FCGR1A, FCGR1B, FCGR2A,
FCGR2C, FCGR3A, FCGR3B, FOLR2, IGSF6, IK, IL18, IL1A, IL1B, IL1RN, IL23A,
LAMP3, LGALS9B, LGALS9C, LILRB4, LILRB5, LY86, LYVE1, MARCO, MERTK,
MNDA, MRC1, MS4A4A, MSR1, OLR1, OTOF, PDCD1LG2, PILRA, RNASE7, SCARB1,
SERPING1, SIGLEC1, SIGLEC14, SIGLEC5, SIGLEC7, SKAP2, SLAMF8, SLPI, STAP2,
TGM2, TLR8, TNF, TNFAIP8L2, TNFSF13B, TNIP3, TYROBP, VSIG4

TABLE 7B
(Overlapping genes in co-expression derived myeloid subpopulations
between 3 compartments)
PBMC/Lung/BAL
ADAMDEC1, AIF1, APOC1, C1QA, C1QB, C1QC, C2, C4BPA, C6, CCL18, CCL2, CCL7,
CCL8, CD80, CLEC4E, CXCL10, CXCL11, FCER1G, FCGR1A, FCGR2A, FCGR2C,
FOLR2, IGSF6, IL18, IL1A, IL1RN, LILRB4, LILRB5, LY86, MNDA, MS4A4A, MSR1,
OTOF, PILRA, SERPING1, SIGLEC5, TNF, TNFSF13B, TYROBP, VSIG4
PBMC/Lung
ADGRE2, APOBEC3B, APOBR, BST1, CD14, CD300E, CD33, CFD, CFP, CLEC12A,
CLEC12B, CLEC4A, CLEC4D, CLEC7A, CSF2RB, CSF3R, CST3, CYBA, CYBB,
FCGR2B, FGR, GRN, IL1RAP, ITGAX, LGALS9, LILRA5, LILRA6, LILRB1, LILRB2,
LILRB3, LMNB1, LYZ, MPEG1, MS4A6A, NLRP3, PLEK, S100A8, S100A9, SECTM1,
SEMA4A, SERPINB9, SPI1, TLR2, TREM1, TREML4, UNC93B1
PBMC/BAL
C5, CD163, CD300LF, CD68, CLEC5A, CLEC6A, CXCL2, CXCL9, FCGR1B, LYVE1,
MARCO, MERTK, MRC1, RNASE7, SCARB1, SIGLEC1, SIGLEC7, SKAP2, TGM2,
TLR8, TNFAIP8L2
Lung/BAL
APOBEC3G, CHI3L1, CLEC2B, CXCL1, CXCL8, CXCR2, FCGR3A, FCGR3B, IL1B,
IL23A, LAMP3, LGALS9C, OLR1, PDCD1LG2, SIGLEC14, SLPI, TNIP3
PBMC
AOAH, C1RL, C4A, C4B, CD101, CD209, CD300C, CLEC10A, CLEC16A, CSF1R,
CSF2RA, FLT3, FUT4, HVCN1, IFI30, IL10RA, LGALS12, LILRA1, LILRA2, MEFV,
MFGE8, NLRP12, NOD2, NTSR1, OSCAR, PECAM1, PRAM1, RNASE3, RNASE6,
SIGLEC10, SLC11A1, SLITRK4, THBD, TNFRSF1B, VENTX, VSTM1
Lung
ADGRE1, C4BPB, C8A, CCL22, CHIT1, CLEC1A, CSF2, CXCL13, FFAR2, IFNL1,
IL12B, IL18RAP, IL20, IL27, S1PR5, TNFSF14
BAL
ACE, ADGRE3, C1R, CCL28, CD5L, IK, LGALS9B, SLAMF8, STAP2

TABLE 7C
(Input genes to trajectory analysis)
AASS, ABCD1, ABCD2, ABCD3, ACAA2, ACACB, ACADM, ACADS, ACADVL,
ACAT1, ACAT2, ACE, ACO2, ACOX1, ACOX2, ACOX3, ACOXL, ACSL1, ACSL5,
ADAM8, ADAMDEC1, ADGRE1, ADGRE2, ADGRE3, ADK, AIF1, AIM2, AK3, AKT2,
ALDH4A1, ALDH7A1, ALOX15, AOAH, APOBEC3B, APOBEC3G, APOBR, APOC1,
APOL1, APOL2, APOL3, APOL6, ARG1, ART4, ATF3, ATP5A1, ATP5B, ATP5D,
ATP5E, ATP5F1, ATP5G1, ATP5G2, ATP5G3, ATP5H, ATP5I, ATP5J, ATP5J2, ATP5L,
ATP5O, ATP5S, AUH, BATF2, BATF3, BCL2A1, BCS1L, BDH2, BIRC3, BMX, BST1,
BTK, C16orf54, C1QA, C1QB, C1QC, C1R, C1RL, C2, C4A, C4B, C4BPA, C4BPB, C5,
C6, C8A, CA2, CASP1, CASP10, CCL13, CCL15, CCL18, CCL19, CCL2, CCL20, CCL22,
CCL23, CCL28, CCL5, CCL7, CCL8, CCND2, CCR7, CD101, CD14, CD163, CD180,
CD209, CD274, CD300A, CD300C, CD300E, CD300LF, CD33, CD36, CD38, CD4, CD40,
CD52, CD5L, CD68, CD72, CD80, CD86, CDKN1A, CEACAM1, CEP89, CERK, CFB,
CFD, CFP, CHI3L1, CHI3L2, CHIT1, CLEC10A, CLEC12A, CLEC12B, CLEC16A,
CLEC1A, CLEC2B, CLEC4A, CLEC4D, CLEC4E, CLEC4F, CLEC5A, CLEC6A,
CLEC7A, CNR2, COA1, COA3, COA4, COA5, COA6, COA7, COX10, COX10-AS1,
COX11, COX14, COX15, COX16, COX17, COX18, COX19, COX20, COX4I1, COX4I2,
COX5A, COX5B, COX6A1, COX6B1, COX6C, COX7A1, COX7A2, COX7A2L, COX7B,
COX7C, COX8A, CPT1A, CPT2, CROT, CS, CSAG3, CSF1R, CSF2, CSF2RA, CSF2RB,
CSF3R, CST3, CTSC, CXCL1, CXCL10, CXCL11, CXCL13, CXCL2, CXCL8, CXCL9,
CXCR2, CXCR4, CYBA, CYBB, CYC1, CYCS, DECR1, DEF6, DLAT, DLC1, DLD,
DLST, DNAJC15, DOCK8, EBI3, ECHDC1, ECHDC2, ECHS1, ECI1, ECI2, EDN1, EGR2,
EHHADH, EPB41, ETFA, ETFB, ETFDH, F8, FAS, FCAR, FCER1A, FCER1G, FCER2,
FCGR1A, FCGR1B, FCGR2A, FCGR2B, FCGR2C, FCGR3A, FCGR3B, FDCSP, FFAR2,
FGL2, FGR, FH, FLT3, FN1, FOLR2, FUT4, FYB1, G6PD, GAD1, GADD45B, GADD45G,
GBP1, GBP2, GCDH, GCH1, GCNT1, GIMAP2, GIMAP4, GIMAP5, GLS, GLUD1, GOT1,
GPR18, GPR84, GRHPR, GRN, H6PD, HAAO, HACL1, HADH, HADHA, HADHB,
HAVCR2, HBG2, HCK, HESX1, HEXB, HHLA2, HIBCH, HLX, HNMT, HRH1, HS3ST1,
HS3ST2, HSD11B1, HSD17B4, HVCN1, IDH1, IDH2, IDH3A, IDH3B, IDH3G, IDO1,
IER3, IFI27, IFI30, IFI44, IFNL1, IGF1, IGFBP4, IGSF6, IK, IL10RA, IL12A, IL12B, IL15,
IL15RA, IL18, IL18R1, IL18RAP, IL1A, IL1B, IL1RAP, IL1RN, IL20, IL23A, IL27,
IL2RA, IL2RG, IL31RA, IL4I1, IL6, IL7R, INHBA, IRF1, IRF7, IRS1, IRS2, ITGAM,
ITGAX, IVD, JAK2, LAIR1, LAMP3, LAP3, LAT, LCP2, LDHAL6B, LGALS12, LGALS9,
LGALS9B, LGALS9C, LILRA1, LILRA2, LILRA5, LILRA6, LILRB1, LILRB2, LILRB3,
LILRB4, LILRB5, LIPA, LMNB1, LPAR6, LTA4H, LY75, LY86, LYVE1, LYZ, MAF,
MARCO, MCCC2, MDH2, MEFV, MERTK, MFGE8, MNDA, MPC1, MPC2, MPEG1,
MRC1, MRPS15, MS4A4A, MS4A6A, MSR1, MT-ATP6, MT-ATP8, MT-CO1, MT-CO2,
MT-CO3, MT-CYB, MT-ND1, MT-ND2, MT-ND3, MT-ND4, MT-ND4L, MT-ND5, MT-
ND6, NAMPT, NDUFA1, NDUFA10, NDUFA11, NDUFA12, NDUFA13, NDUFA2,
NDUFA3, NDUFA4, NDUFA4L2, NDUFA5, NDUFA6, NDUFA8, NDUFA9, NDUFAB1,
NDUFAF1, NDUFAF2, NDUFAF3, NDUFAF4, NDUFAF5, NDUFAF6, NDUFAF7,
NDUFAF8, NDUFB1, NDUFB10, NDUFB11, NDUFB2, NDUFB3, NDUFB4, NDUFB5,
NDUFB6, NDUFB7, NDUFB8, NDUFB9, NDUFC1, NDUFC2, NDUFS1, NDUFS2,
NDUFS3, NDUFS4, NDUFS5, NDUFS6, NDUFS7, NDUFS8, NDUFV1, NDUFV2,
NDUFV3, NKTR, NLRP12, NLRP3, NOD2, NR3C1, NTSR1, NUBPL, OAS1, OAS2,
OAS3, OASL, OAT, OGDH, OLR1, OSCAR, OTOF, OXA1L, OXCT1, P2RY13, P2RY14,
PC, PDCD1LG2, PDGFA, PDHA1, PDHB, PDHX, PDK1, PDK2, PDK3, PDK4, PDP1,
PDP2, PDPR, PECAM1, PEX13, PEX2, PEX5, PEX7, PFKFB2, PFKFB3, PFKFB4, PFKP,
PGD, PHYH, PIK3AP1, PILRA, PKM, PLA1A, PLA2G5, PLEK, POU2F2, PRAM1, PRPS1,
PRPS2, PSAT1, PSMA2, PSMB9, PSME2, PSTPIP1, PTCH1, PTPN7, PTX3, PYHIN1,
RBKS, RGN, RGS1, RNASE3, RNASE6, RNASE7, RPE, RPIA, S100A8, S100A9, S1PR5,
SAMSN1, SCARB1, SCO1, SDHA, SDHAF1, SDHAF2, SDHAF3, SDHAF4, SDHB,
SDHC, SDHD, SDS, SECTM1, SELENOP, SEMA4A, SERPINB9, SERPING1, SESN2,
SIGLEC1, SIGLEC10, SIGLEC14, SIGLEC5, SIGLEC7, SKAP2, SLAMF7, SLAMF8,
SLC11A1, SLC25A17, SLC25A4, SLC27A2, SLC2A1, SLC2A3, SLC2A4, SLC2A5,
SLC2A6, SLC31A2, SLC38A6, SLC4A7, SLC7A5, SLCO2B1, SLCO5A1, SLITRK4, SLPI,
SOCS1, SP100, SPHK1, SPI1, SRGN, STAP2, STAT1, STAT2, STX11, SUCLA2,
SUCLG1, SUCLG2, SURF1, TACO1, TALDO1, TAP1, TAP2, TEK, TGFBI, TGFBR2,
TGM2, THBD, THEMIS2, TIMMDC1, TKT, TKTL1, TLR1, TLR2, TLR5, TLR8,
TMEM126B, TMEM154, TNF, TNFAIP8L2, TNFRSF11A, TNFRSF1B, TNFSF10,
TNFSF13B, TNFSF14, TNFSF4, TNFSF8, TNFSF9, TNIP3, TPST2, TRAP1, TREM1,
TREML2, TREML4, TTC19, TYROBP, UBE2L6, ULBP1, ULBP2, UNC93B1, UQCC1,
UQCC2, UQCC3, UQCR10, UQCR11, UQCRB, UQCRC1, UQCRC2, UQCRFS1, UQCRH,
UQCRHL, UQCRQ, VCAM1, VCAN, VENTX, VSIG4, VSTM1, WARS, XAF1

TABLE 8A
(Drugs and compounds targeting IPA upstream regulators via blood,
lung, or airway)
PBMC-CoV2 vs. PBMC-CTL (Blood)
IFNG: Amg 811‡
CSF1: Sunitinib†
Anti-IL6: Imiquimod†, PF-04236921‡, Siltuximab†, Sirukumab‡, Sarilumab†, Tocilizumab†,
Vobarilizumab‡
Anti-IFNA: AGS-009‡, Rontalizumab‡, Sifalimumab‡, Anifrolumab‡
CD38: Daratumumab†, TAK-079‡
TNKS: XAV-939^{P}
TGFB1: SB-431542^{P}, SB-525334^{P},
EGFR inhibitors: Brigatinib†, Dacomitinib†, Erlotinib†, Gefitinib†, Lapatinib†, Osimertinib†,
Vandetanib†, CGP-52411^{P}, Afatinib†, AG-490^{P}, AG-494^{P}, BIBU-1361^{P} BIBX-1382‡,
Butein^{P}, CP-724714‡, Erbstatin-analog^{P}, Lavendustin-a, Lavendustin-c, RG-13022,
Tyrphostin, TYRPHOSTIN-1, TYRPHOSTIN-AG-1478^{P}, TYRPHOSTIN-AG-18^{P},
TYRPHOSTIN-AG-494^{P}, TYRPHOSTIN-AG-555, TYRPHOSTIN-AG-82, TYRPHOSTIN-
AG-835^{P}, WZ-3146^{P}, WZ-4002^{P}, Neratinib†, AEE788‡, ARRY-334543‡, AST-1306‡, AV-
412‡, AZD8931‡, BMS-599626‡, BMS-690514‡, Canertinib‡, CUDC-101‡, FERb-033^{P},
GW-583340^{P}, Mubritinib‡, PD-168393^{P}, Poziotinib‡, TAK-285‡, Tucatinib‡, Tyrphostin-AG-
825^{P}, Tyrphostin-AG-879^{P}, XL647‡
ERBB2: EGFR inhibitors
INSR inhibitors: Ceritinib†, Dovitinib‡, GSK1838705A^{P}, GSK1904529A^{P}, Linsitinib‡, NVP-
AEW541^{P}, NVP-TAE684^{P}
BRD4: LY-303511^{P}, AT-7519‡, Alvocidib‡, Roscovitine‡
VEGFR inhibitors: Lenvatinib†, Pazopanib†, Sorafenib†, Sunitinib†, ZM-306416^{P}
AKT inhibitors: 10-DEBC^{P}, A-443644, BML-257, Deguelin, Lonidamine†, Miltefosine†,
MK-2206‡
PPARG: Acetyl-farnesyl-cysteine†, T-0070907^{P}
PPARA: 2-fluoropalmitic-acid, GW-6471^{P}
HIF1A: CAY-10585, YC-1^{P}, 1-Benzylimidazole
STAT1: Sinensetin
IGF1R inhibitors: BMS-536924^{P}, BMS-754807‡, Ceritinib†, GSK-1904529A^{P}, Linsitinib‡,
PQ-401^{P}
BAX: BAX-Channel-Blocker
Drugs Predicted As Upstream Regulators (Inhibited): GnRH analog, Levodopa†, MEL S3,
Calcitriol†, CD 437, Ciprofloxacin†, ST1926, 5-fluorouracil†, Sirolimus†,
Medroxyprogesterone acetate†, Valproic acid†, BMS-690514‡, Panobinostat†, BMS-754807‡
Lung-CoV2 vs. Lung-CTL (Lung)
NFxB pathway inhibitors: CAY-10470^{P}, Quinacrine†, Quinoclamine, Auranofin†, BAY-11-
7082^{P}, BAY-11-7085^{P}, Betulinic-acid‡, Bicyclol†, Bindarit‡, Bortezomib†, Cepharanthine‡,
CKD-712‡, Closantel†, Curcumin†, Edasalonexent†, Erythromycin†, EVP4593^{P}, E3330^{P},
Ginsenoside-C-K‡, Hexamethylenebisacetamide‡, Iguratimod†, IKK-2-inhibitor-V‡, MD-
920‡, NFKB-activation-inhibitor-II^{P}, Parthenolide‡, Parthenolide-(-)^{P}, Parthenolide-
(alternate-stereo)^{P}, Pyrrolidine-dithiocarbamate^{P}, Ro-106-9920^{P}, Sasapyrine†, Sesamin^{P}, SP-
100030^{P}, Ursolic-acid‡, Verbascoside‡
IL12: Ustekinumab†
Lymphotoxin: Etanercept†
IFNG: Amg 811‡
Anti-IL17: Brodalumab†, Ixekizumab†, Secukinumab†
Anti-TNF: Etanercept†, Adalimumab†, Certolizumab pegol†, Golimumab†, Infliximab†
Anti-IFNA: AGS-009‡, Rontalizumab‡, Sifalimumab‡, Anifrolumab‡
Anti-IL1: Canakinumab†, Anakinra†
IFNB1: PF-06823859‡
TNFSF12: BIIB023‡
TGM2: GK921^{P}, LDN-27219^{P}
RNASE1: Citric acid^{P}, Guanidine†, L-aspartic-acid†
APC: JW-67^{P}
NOS2: AR-C133057XX, Diphenyleneiodonium^{P}, Pyrrolidine-dithiocarbamate^{P}
ETV6-NTRK3: Entrectinib†, GNF-5837^{P}, Larotrectinib†
Interferon: Anti-IFNA, Amg 811†, Anti-IFN OMEGA‡, PF-06823859‡
JAK inhibitors: AT-9283‡, Baricitinib†, Cucurbitacin-I, Filgotinib‡, Ruxolitinib†, Solcitinib‡,
Tofacitinib†, Upadacitinib†, AG-490^{P}, Atiprimod, Atractylenolide-i^{P}, AZD1480‡, AZ960^{P},
BMS-911543‡, CEP-33779^{P}, Curcumol‡, cyt387‡, Decernotinib‡, Delgocitinib‡, Fedratinib†,
Ganoderic-acid-a^{P}, Itacitinib‡, JAK3-inhibitor-V^{P}, LY2784544‡, NS-018‡, NVP-BSK805^{P},
Pacritinib‡, Peficitinib†, PF-06651600‡, Ruxolitinib-(S)^{P}, TCS-21311^{P}, TG-02‡, TG-
101209^{P}, Thiram†, WHI-P154^{P}, XL019‡, ZM-39923^{P}, 1,2,3,4,5,6-hexabromocyclohexane^{P}
Tlr: Hydroxychloroquine†
Tnf (family) inhibitors: Anti-TNF, Dapirolizumab pegol‡, BIIB023t, Atacicept‡, RC18‡,
Amg 570‡, Belimumab†, Blisibimod‡, Tabalumab‡
Drugs Predicted As Upstream Regulators (Inhibited): Alefacept, Etanercept†, Fontolizumab,
Leuprolide†, Hydrogen peroxide, AT^{P}, Hydroquinone†, Nitric oxide, Estriol†,
Norepinephrine†, D-glucose, Sphingosine-1-phosphate, Dexamethasone†,
Methylprednisolone†, Raloxifene†, 3-deazaneplanocin^{P}, Dalfampridine†,
Medroxyprogesterone acetate†. Lithium chloride, Chloropromazine, Tofacitinib†,
Nelfinavir†, Chloroquine†, Quercetin†
BAL-CoV2 vs. PBMC-CoV2 (Airway)
Anti-IL17: Brodalumab†, Ixekizumab†, Secukinumab†
CD3: Blinatumomab†
FCER1: Omalizumab
PXR-ligand-PXR-Retinoic acid-RXRα: Retinol†, Fenretinide†
IL13: Lebrikizumab†
IL2: Daclizumab†
FLT3LG: Midostaurin†, Quizartinib‡, Sorafenib†, TCS-359^{P}
IL3: Talacotuzumab†
LIPE: Oxybenzonet†, Benzyl-benzoate†
B4GALNT1: Dinutuximab†
SIRT3: Tenovin-6^{P}
RNASE1: Citric acid^{P}, Guanidine†, L-aspartic-acid†
EWSR1-FLI1: YK-4-279^{P}
Nr1h: GW-4064^{P}
Anti-Hsp90: Alvespimycin‡, BIIB021‡, Gedunin^{P}, Geldanamycin^{P}, NVP-AUY922‡, PU-
H71‡, Radicicol, Tanespimycin‡
IL12: Ustekinumab†
JAK inhibitors: AT-9283‡, Baricitinib†, Cucurbitacin-I, Filgotinib‡, Ruxolitinib†, Solcitinib‡,
Tofacitinib†, Upadacitinib†, AG-490^{P}, Atiprimod‡, Atractylenolide-i^{P}, AZD1480‡, AZ960^{P},
BMS-911543‡, CEP-33779^{P}, Curcumol‡, cyt387‡, Decernotinib‡, Delgocitinib‡, Fedratinib†,
Ganoderic-acid-a^{P}, Itacitinib‡, JAK3-inhibitor-V^{P}, LY2784544‡, NS-018‡, NVP-BSK805^{P},
Pacritinib, Peficitinib‡, PF-06651600‡, Ruxolitinib-(S)^{P}, TCS-21311^{P}, TG-02‡, TG-
101209^{P}, Thiram†, WHI-P154^{P}, XL019t, ZM-39923^{P}, 1,2,3,4,5,6-hexabromocyclohexane^{P}
PRKCQ: Afimoxifene‡, Dexfosfoserine^{P}, GSK690693‡, Sotrastaurin‡, Tamoxifen†
LCK: Aminogenistein, JW-7-24-1, PP-2^{P}, WH-4023
ROCK inhibitors: Fasudil†, Ripasudil†, GSK-429286A^{P}, GSK-269962^{P}, KD025‡, LX7101‡,
OXA-06^{P}, RKI-1447^{P}, SB-772077B^{P}, SR-3677^{P}, Y-39983†, Y-27632^{P}
Estrogen receptor antagonists: Tamoxifen†, Acolbifene‡, Afimoxifene‡, Clomifene†,
Danazol†, Endoxifen‡, Fulvestrant†, G-15^{P}, GDC-0927‡, PHTPP^{P}, Raloxifene†,
Toremifene†, Y-134^{P}, ZK-164015^{P}
AR: Androstenedione, BMS-641988, Enzalutamide†, Flutamide†, Nilutamide†
SNH: SANT-1^{P}, SANT-2^{P}
STATE Sinensetin
EPAS1: Cephaeline, YC-1^{P}, TC-S-7009^{P}, PT-2385‡
GATA3: Cyclosporin-a†, Simvastatin†, Tacrolimus†
TEAD2: Oleoylethanolamide^{P}
IKZF3: Lenalidomide†
ATF2: Ephedrine-(racemic)†
ITGB2: BMS-587101‡, Simvastatin†
TLR3: CU-CPT-4a^{P}
Anti-IL6: Imiquimod†, PF-04236921‡, Siltuximab†, Sirukumab‡, Sarilumab†, Tocilizumab†,
Vobarilizumab‡
Drugs Predicted As Upstream Regulators (Inhibited): 15-deoxy-delta-12, 14-PGJ 2,
Hydrocortisone†, SB203580, JAK inhibitor I, UM101, Z-DEVD-FMK, Triptolide‡,
Etretinate\hat{ }
Legend: †FDA-approved; ‡Drug in development/clinical trials; ^{P}Preclinical; \hat{ }Withdrawn from market

TABLE 8B
(Unique IPA-predicted drugs by mechanism of action in blood, lung, or airway)
PBMC (blood)
Abl kinase inhibitor, ACE inhibitor, activator of soluble guanylyl cyclase, ALK tyrosine
kinase receptor mutant inhibitor, Anti-CD38, Anti-IFNAR1, Anti-IFNG, Anti-IL6, Anti-IL6
receptor, Anti-TNF, apoptosis stimulant, bacterial DNA gyrase inhibitor, Benzodiazepine
receptor agonist, CCR expression inhibitor, cell cycle inhibitor, COX inhibitor, CSFR
antagonist, cytochrome C release inhibitor, dopamine precursor, ErbB2 tyrosine kinase
inhibitor, FGFR inhibitor, glucokinase inhibitor, GnRH agonist, hypercalcaemic agent,
hypoxia inducible factor inhibitor, IFNA inhibitor, inhibitor of methylation of endogenous
isoprenylated proteins, insulin growth factor receptor inhibitor, insulin receptor ligand,
interferon inducer, interleukin inhibitor, kinase inhibitor, KIT inhibitor, Leucine rich repeat
kinase inhibitor, MCL1 inhibitor, membrane integrity inhibitor, NADH-ubiquinone
oxidoreductase (Complex I) inhibitor, NFκB pathway inhibitor, PDGFR inhibitor, phorbol
ester-induced ornithine decarboxylase (ODC) activity suppressor, PPAR receptor antagonist,
Proteasome inhibitor, RAF inhibitor, receptor tyrosine protein kinase inhibitor, RET tyrosine
kinase inhibitor, SIRT activator, Src inhibitor, Src tyrosine kinase inhibitor, steroid 5alpha-
reductase inhibitor, survivin inhibitor, T cell inhibitor, TGF beta receptor inhibitor,
thromboxane synthase inhibitor, thymidylate synthase inhibitor, TLR agonist, TNKS
inhibitor, tyrosine kinase inhibitor, VEGFR inhibitor, vitamin D receptor agonist, XIAP
inhibitor
Lung
Abl kinase inhibitor, acetylcholinesterase inhibitor, adiponectin receptor agonist, adrenergic
receptor agonist, aldehyde dehydrogenase inhibitor, aldose reductase inhibitor, algicide, ALK
expression enhancer, Anti-CD2, Anti-CD40LG, Anti-ICOSLG, Anti-IFNAR1, Anti-IFNB1,
Anti-IFNG, Anti-IFNW1, Anti-IL1, Anti-IL17, Anti-IL17 receptor, antimalarial agent, Anti-
TNF, Anti-TNFSF12, Anti-TNFSF13, Anti-TNFSF13B, AP inhibitor, apoptosis stimulant,
Bcr-Abl kinase inhibitor, CCN expression inhibitor, chitinase inhibitor, cholesterol inhibitor,
coagulation factor inhibitor, corticosteroid agonist, COX inhibitor, cytochrome P450 inhibitor,
cytokine production inhibitor, diacylglycerol O acyltransferase inhibitor, differentiation
inducer, disease modifying antirheumatic drug, DNA inhibitor, DNA methyltransferase
inhibitor, endoglin expression enhancer, ErbB2 tyrosine kinase inhibitor, Estrogen receptor
agonist, free radical scavenger, FtsZ inhibitor, glucocorticoid receptor modulator, glucose 6
phosphatase inhibitor, glucosidase inhibitor, GnRH agonist, growth factor receptor inhibitor,
histone lysine methyltransferase inhibitor, histone N-acetyltransferase inhibitor, HIV integrase
inhibitor, HIV protease inhibitor, IFNA inhibitor, IL12/23 inhibitor, immunosuppressant,
lipocortin synthesis stimulant, lipoxygenase inhibitor, MAPK inhibitor, melanin inhibitor,
metallic radical formation stimulant, mitotic inhibitor, monoamine oxidase inhibitor, NADPH
oxidase inhibitor, NFκB pathway activator, NFκB pathway inhibitor, nitric oxide synthase
inhibitor, phospholipase inhibitor, polar auxin transport inhibitor, potassium channel blocker,
prostanoid receptor antagonist, Proteasome inhibitor, proto-oncogene tyrosine protein kinase
inhibitor, quorum sensing signaling modulator, SARS coronavirus 3C-like protease inhibitor,
selective estrogen receptor modulator, SIRT activator, sodium channel blocker, STAT
inhibitor, steroidal receptor agonist, tau aggregation inhibitor, thioredoxin reductase inhibitor,
tissue transglutaminase inhibitor, TP53 activator, transglutaminase inhibitor, tropomyosin
receptor kinase inhibitor, tyrosine kinase inhibitor, WNT pathway inhibitor, xanthine oxidase
inhibitor
BAL (airway)
Abl kinase inhibitor, activator of soluble guanylyl cyclase, adrenergic receptor agonist, ALK
expression enhancer, Anti-B4GALNT1, Anti-CD19, Anti-CD3, Anti-FCER1A, Anti-IL13,
Anti-IL17, Anti-IL17 receptor, Anti-IL2R, Anti-IL3R, Anti-IL6, Anti-IL6 receptor, Anti-
MS4A2, Anti-TNF, apoptosis stimulant, AR antagonist, ATP citrase lyase inhibitor, Bcr-Abl
kinase inhibitor, binding of RNA helicase A to the transcription factor EWS-FLI1 inhibitor,
calcium channel activator, calcium sensitizer, cell cycle inhibitor, cereblon inhibitor,
cholesterol inhibitor, coagulation factor inhibitor, colony stimulating factor, corticosteroid
agonist, COX inhibitor, cyclin inhibitor, cyclophilin inhibitor, cytochrome P450 inhibitor,
disease modifying antirheumatic drug, DNA directed DNA polymerase inhibitor, endoglin
expression enhancer, ErbB2 tyrosine kinase inhibitor, Estrogen receptor agonist, estrogen
receptor modulator, EWS-FLI1 inhibitor, FK506-binding protein inhibitor, FXR agonist,
glucose-dependent insulinotropic receptor agonist, histamine release inhibitor, hypoxia
inducible factor inhibitor, IL12/23 inhibitor, immunostimulant, immunosuppressant, insulin
expression inhibitor, integrin antagonist, interferon inducer, interleukin inhibitor, kinase
inhibitor, KIT inhibitor, LCK inhibitor, Leucine rich repeat kinase inhibitor, LIM kinase
inhibitor, lipase inhibitor, lipocortin synthesis stimulant, luteinizing hormone releasing
hormone antagonist, macrolide calcineurin inhibitor, MAPK inhibitor, membrane integrity
inhibitor, metallic radical formation stimulant, mitotic inhibitor, multi targeted kinase
inhibitor, opioid receptor ligand, PKA inhibitor, potassium channel blocker, PPAR receptor
agonist, PPAR receptor antagonist, protease inhibitor, pyruvate dehydrogenase kinase
inhibitor, RAF inhibitor, RET tyrosine kinase inhibitor, retinoid receptor agonist, RNA
directed RNA polymerase inhibitor, ROCK inhibitor, rotamase inhibitor, selective estrogen
receptor modulator, SIRT inhibitor, soluble epoxide hydrolase inhibitor, Src inhibitor, STAT
inhibitor, steroid derivative with antigonadotropic and anti-estrogenic activities, stress
activated protein kinase inhibitor, T cell inhibitor, testosterone receptor agonist, TLR agonist,
TLR inhibitor, TNF modulator, tumor apoptosis inducer, tyrosine kinase inhibitor, VEGFR
inhibitor
Legend: † FDA-approved; ‡ Drug in development/clinical trials; ^{P} Preclinical; \hat{ } Withdrawn from market

Further details are described by, for example, Daamen et al., “Comprehensive transcriptomic analysis of COVID-19 blood, lung, and airway”, Scientific Reports, Vol. 11, No. 7052 (2021), which is incorporated by reference herein in its entirety.

While preferred embodiments have been shown and described herein, it will be obvious to those skilled in the art that such embodiments are provided by way of example only. Numerous variations, changes, and substitutions will now occur to those skilled in the art without departing from the scope of the disclosure. It should be understood that various alternatives to the embodiments described herein may be employed in practice. Numerous different combinations of embodiments described herein are possible, and such combinations are considered part of the present disclosure. In addition, all features discussed in connection with any one embodiment herein can be readily adapted for use in other embodiments herein. It is intended that the following claims define the scope of the disclosure and that methods and structures within the scope of these claims and their equivalents be covered thereby.

What is claimed is:

1. A method for determining a COVID-19 disease state of a subject, comprising:
(a) assaying a biological sample obtained or derived from the subject to produce a data set comprising gene expression measurements of the biological sample at each of a plurality of COVID-19 disease-associated genomic loci, wherein the plurality of COVID-19 disease-associated genomic loci comprises at least a portion of a gene selected from the group of genes listed in Table 6 and Tables 7A-7C;
(b) computer processing the data set to determine the COVID-19 disease state of the subject; and
(c) electronically outputting a report indicative of the COVID-19 disease state of the subject.

2. The method of claim 1, wherein the plurality of COVID-19 disease-associated genomic loci comprises at least a portion of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 350, 400, 450, 500, 550, 600, 650, 700, 750, 800, 850, 900, 950, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, or 6000 genes selected from the group of genes listed in Table 6 and Tables 7A-7C.

3. The method of claim 1, further comprising determining the COVID-19 disease state of the subject with an accuracy of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

4. The method of claim 1, further comprising determining the COVID-19 disease state of the subject with a sensitivity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

5. The method of claim 1, further comprising determining the COVID-19 disease state of the subject with a specificity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

6. The method of claim 1, further comprising determining the COVID-19 disease state of the subject with a positive predictive value of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

7. The method of claim 1, further comprising determining the COVID-19 disease state of the subject with a negative predictive value of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

8. The method of claim 1, further comprising determining the COVID-19 disease state of the subject with an Area-Under-Curve (AUC) of at least about 0.50, at least about 0.55, at least about 0.60, at least about 0.65, at least about 0.70, at least about 0.75, at least about 0.80, at least about 0.85, at least about 0.90, at least about 0.91, at least about 0.92, at least about 0.93, at least about 0.94, at least about 0.95, at least about 0.96, at least about 0.97, at least about 0.98, at least about 0.99, or more than about 0.99.

9. The method of claim 1, wherein the subject has received a diagnosis of the COVID-19 disease.

10. The method of claim 1, wherein the subject is suspected of having the COVID-19 disease.

11. The method of claim 1, wherein the subject is at elevated risk of having the COVID-19 disease or having severe complications from the COVID-19 disease.

12. The method of claim 1, wherein the subject is asymptomatic for the COVID-19 disease.

13. The method of any one of claims 1 to 12, further comprising administering a treatment to the subject based at least in part on the determined COVID-19 disease state.

14. The method of claim 13, wherein the treatment is configured to treat the COVID-19 disease state of the subject.

15. The method of claim 13, wherein the treatment is configured to reduce a severity of the COVID-19 disease state of the subject.

16. The method of claim 13, wherein the treatment is configured to reduce a risk of having the COVID-19 disease.

17. The method of claim 13, wherein the treatment comprises a drug.

18. The method of claim 17, wherein the drug is selected from the group listed in Tables 8A-8B.

19. The method of claim 1, wherein (b) comprises using a trained machine learning classifier to analyze the data set to determine the COVID-19 disease state of the subject.

20. The method of claim 19, wherein the trained machine learning classifier is trained using gene expression data obtained by a data analysis tool selected from the group consisting of: a BIG-C™ big data analysis tool, an I-Scope™ big data analysis tool, a T-Scope™ big data analysis tool, a CellScan big data analysis tool, an MS (Molecular Signature) Scoring™ analysis tool, and a Gene Set Variation Analysis (GSVA) tool.

21. The method of claim 19, wherein the trained machine learning classifier is selected from the group consisting of a linear regression, a logistic regression, a Ridge regression, a Lasso regression, an elastic net (EN) regression, a support vector machine (SVM), a gradient boosted machine (GBM), a k nearest neighbors (kNN), a generalized linear model (GLM), a naïve Bayes (NB) classifier, a neural network, a Random Forest (RF), a deep learning algorithm, and a combination thereof.

22. The method of claim 1, wherein (b) comprises comparing the data set to a reference data set.

23. The method of claim 22, wherein the reference data set comprises gene expression measurements of reference biological samples at each of the plurality of COVID-19 disease-associated genomic loci.

24. The method of claim 23, wherein the reference biological samples comprise a first plurality of biological samples obtained or derived from subjects having the COVID-19 disease and a second plurality of biological samples obtained or derived from subjects not having the COVID-19 disease.

25. The method of claim 1, wherein the biological sample is selected from the group consisting of: a blood sample, isolated peripheral blood mononuclear cells (PBMCs), a biopsy sample, and any derivative thereof.

26. The method of any one of claims 1-25, further comprising determining a likelihood of the determined COVID-19 disease state.

27. The method of any one of claims 1 to 26, further comprising monitoring the COVID-19 disease state of the subject, wherein the monitoring comprises assessing the COVID-19 disease state of the subject at a plurality of time points.

28. The method of claim 27, wherein a difference in the assessment of the COVID-19 disease state of the subject among the plurality of time points is indicative of one or more clinical indications selected from the group consisting of: (i) a diagnosis of the COVID-19 disease state of the subject, (ii) a prognosis of the COVID-19 disease state of the subject, and (iii) an efficacy or non-efficacy of a course of treatment for treating the COVID-19 disease state of the subject.

29. A computer system for determining a COVID-19 disease state of a subject, comprising:
a database that is configured to store a dataset comprising gene expression data, wherein the gene expression data is obtained by assaying a biological sample obtained or derived from the subject to produce gene expression measurements of the biological sample at each of a plurality of COVID-19 disease-associated genomic loci, wherein the plurality of COVID-19 disease-associated genomic loci comprises at least a portion of a gene selected from the group of genes listed in Table 6 and Tables 7A-7C; and
one or more computer processors operatively coupled to the database, wherein the one or more computer processors are individually or collectively programmed to: (i) computer process the data set to determine the COVID-19 disease state of the subject; (ii) electronically output a report indicative of the COVID-19 disease state of the subject.

30. The computer system of claim 29, further comprising an electronic display operatively coupled to the one or more computer processors, wherein the electronic display comprises a graphical user interface that is configured to display the report.

31. The computer system of claim 29, wherein the plurality of COVID-19 disease-associated genomic loci comprises at least a portion of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 350, 400, 450, 500, 550, 600, 650, 700, 750, 800, 850, 900, 950, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, or 6000 genes selected from the group of genes listed in Table 6 and Tables 7A-7C.

32. The computer system of claim 29, wherein the one or more computer processors are individually or collectively programmed to further determine the COVID-19 disease state of the subject with an accuracy of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

33. The computer system of claim 29, wherein the one or more computer processors are individually or collectively programmed to further determine the COVID-19 disease state of the subject with a sensitivity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

34. The computer system of claim 29, wherein the one or more computer processors are individually or collectively programmed to further determine the COVID-19 disease state of the subject with a specificity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

35. The computer system of claim 29, wherein the one or more computer processors are individually or collectively programmed to further determine the COVID-19 disease state of the subject with a positive predictive value of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

36. The computer system of claim 29, wherein the one or more computer processors are individually or collectively programmed to further determine the COVID-19 disease state of the subject with a negative predictive value of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

37. The computer system of claim 29, wherein the one or more computer processors are individually or collectively programmed to further determine the COVID-19 disease state of the subject with an Area-Under-Curve (AUC) of at least about 0.50, at least about 0.55, at least about 0.60, at least about 0.65, at least about 0.70, at least about 0.75, at least about 0.80, at least about 0.85, at least about 0.90, at least about 0.91, at least about 0.92, at least about 0.93, at least about 0.94, at least about 0.95, at least about 0.96, at least about 0.97, at least about 0.98, at least about 0.99, or more than about 0.99.

38. The computer system of claim 29, wherein the subject has received a diagnosis of the COVID-19 disease.

39. The computer system of claim 29, wherein the subject is suspected of having the COVID-19 disease.

40. The computer system of claim 29, wherein the subject is at elevated risk of having the COVID-19 disease or having severe complications from the COVID-19 disease.

41. The computer system of claim 29, wherein the subject is asymptomatic for the COVID-19 disease.

42. The computer system of any one of claims 29-41, wherein the one or more computer processors are individually or collectively programmed to further direct a treatment to be administered to the subject based at least in part on the determined COVID-19 disease state.

43. The computer system of claim 42, wherein the treatment is configured to treat the COVID-19 disease state of the subject.

44. The computer system of claim 42, wherein the treatment is configured to reduce a severity of the COVID-19 disease state of the subject.

45. The computer system of claim 42, wherein the treatment is configured to reduce a risk of having the COVID-19 disease.

46. The computer system of claim 42, wherein the treatment comprises a drug.

47. The computer system of claim 46, wherein the drug is selected from the group listed in Tables 8A-8B.

48. The computer system of claim 29, wherein (i) comprises using a trained machine learning classifier to analyze the data set to determine the COVID-19 disease state of the subject.

49. The computer system of claim 48, wherein the trained machine learning classifier is trained using gene expression data obtained by a data analysis tool selected from the group consisting of: a BIG-C™ big data analysis tool, an I-Scope™ big data analysis tool, a T-Scope™ big data analysis tool, a CellScan big data analysis tool, an MS (Molecular Signature) Scoring™ analysis tool, and a Gene Set Variation Analysis (GSVA) tool.

50. The computer system of claim 48, wherein the trained machine learning classifier is selected from the group consisting of a linear regression, a logistic regression, a Ridge regression, a Lasso regression, an elastic net (EN) regression, a support vector machine (SVM), a gradient boosted machine (GBM), a k nearest neighbors (kNN), a generalized linear model (GLM), a naïve Bayes (NB) classifier, a neural network, a Random Forest (RF), a deep learning algorithm, and a combination thereof.

51. The computer system of claim 29, wherein (i) comprises comparing the data set to a reference data set.

52. The computer system of claim 51, wherein the reference data set comprises gene expression measurements of reference biological samples at each of the plurality of COVID-19 disease-associated genomic loci.

53. The computer system of claim 52, wherein the reference biological samples comprise a first plurality of biological samples obtained or derived from subjects having the COVID-19 disease and a second plurality of biological samples obtained or derived from subjects not having the COVID-19 disease.

54. The computer system of claim 29, wherein the biological sample is selected from the group consisting of: a blood sample, isolated peripheral blood mononuclear cells (PBMCs), a biopsy sample, and any derivative thereof.

55. The computer system of any one of claims 29-54, wherein the one or more computer processors are individually or collectively programmed to further determine a likelihood of the determined COVID-19 disease state.

56. The computer system of any one of claims 29-55, wherein the one or more computer processors are individually or collectively programmed to further monitor the COVID-19 disease state of the subject, wherein the monitoring comprises assessing the COVID-19 disease state of the subject at a plurality of time points.

57. The computer system of claim 56, wherein a difference in the assessment of the COVID-19 disease state of the subject among the plurality of time points is indicative of one or more clinical indications selected from the group consisting of: (i) a diagnosis of the COVID-19 disease state of the subject, (ii) a prognosis of the COVID-19 disease state of the subject, and (iii) an efficacy or non-efficacy of a course of treatment for treating the COVID-19 disease state of the subject.

58. A non-transitory computer readable medium comprising machine-executable code that, upon execution by one or more computer processors, implements a method for determining a COVID-19 disease state of a subject, the method comprising:
(a) assaying a biological sample obtained or derived from the subject to produce a data set comprising gene expression measurements of the biological sample at each of a plurality of COVID-19 disease-associated genomic loci, wherein the plurality of COVID-19 disease-associated genomic loci comprises at least a portion of a gene selected from the group of genes listed in Table 6 and Tables 7A-7C;
(b) computer processing the data set to determine the COVID-19 disease state of the subject; and
(c) electronically outputting a report indicative of the COVID-19 disease state of the subject.

59. The non-transitory computer readable medium of claim 58, wherein the plurality of COVID-19 disease-associated genomic loci comprises at least a portion of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 350, 400, 450, 500, 550, 600, 650, 700, 750, 800, 850, 900, 950, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, or 6000 genes selected from the group of genes listed in Table 6 and Tables 7A-7C.

60. The non-transitory computer readable medium of claim 58, further comprising determining the COVID-19 disease state of the subject with an accuracy of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

61. The non-transitory computer readable medium of claim 58, further comprising determining the COVID-19 disease state of the subject with a sensitivity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

62. The non-transitory computer readable medium of claim 58, further comprising determining the COVID-19 disease state of the subject with a specificity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

63. The non-transitory computer readable medium of claim 58, further comprising determining the COVID-19 disease state of the subject with a positive predictive value of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

64. The non-transitory computer readable medium of claim 58, further comprising determining the COVID-19 disease state of the subject with a negative predictive value of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

65. The non-transitory computer readable medium of claim 58, further comprising determining the COVID-19 disease state of the subject with an Area-Under-Curve (AUC) of at least about 0.50, at least about 0.55, at least about 0.60, at least about 0.65, at least about 0.70, at least about 0.75, at least about 0.80, at least about 0.85, at least about 0.90, at least about 0.91, at least about 0.92, at least about 0.93, at least about 0.94, at least about 0.95, at least about 0.96, at least about 0.97, at least about 0.98, at least about 0.99, or more than about 0.99.

66. The non-transitory computer readable medium of claim 58, wherein the subject has received a diagnosis of the COVID-19 disease.

67. The non-transitory computer readable medium of claim 58, wherein the subject is suspected of having the COVID-19 disease.

68. The non-transitory computer readable medium of claim 58, wherein the subject is at elevated risk of having the COVID-19 disease or having severe complications from the COVID-19 disease.

69. The non-transitory computer readable medium of claim 58, wherein the subject is asymptomatic for the COVID-19 disease.

70. The non-transitory computer readable medium of any one of claims 58-69, further comprising directing a treatment to be administered to the subject based at least in part on the determined COVID-19 disease state.

71. The non-transitory computer readable medium of claim 70, wherein the treatment is configured to treat the COVID-19 disease state of the subject.

72. The non-transitory computer readable medium of claim 70, wherein the treatment is configured to reduce a severity of the COVID-19 disease state of the subject.

73. The non-transitory computer readable medium of claim 70, wherein the treatment is configured to reduce a risk of having the COVID-19 disease.

74. The non-transitory computer readable medium of claim 70, wherein the treatment comprises a drug.

75. The non-transitory computer readable medium of claim 74, wherein the drug is selected from the group listed in Tables 8A-8B.

76. The non-transitory computer readable medium of claim 58, wherein (b) comprises using a trained machine learning classifier to analyze the data set to determine the COVID-19 disease state of the subject.

77. The non-transitory computer readable medium of claim 76, wherein the trained machine learning classifier is trained using gene expression data obtained by a data analysis tool selected from the group consisting of: a BIG-C™ big data analysis tool, an I-Scope™ big data analysis tool, a T-Scope™ big data analysis tool, a CellScan big data analysis tool, an MS (Molecular Signature) Scoring™ analysis tool, and a Gene Set Variation Analysis (GSVA) tool.

78. The non-transitory computer readable medium of claim 76, wherein the trained machine learning classifier is selected from the group consisting of a linear regression, a logistic regression, a Ridge regression, a Lasso regression, an elastic net (EN) regression, a support vector machine (SVM), a gradient boosted machine (GBM), a k nearest neighbors (kNN), a generalized linear model (GLM), a naïve Bayes (NB) classifier, a neural network, a Random Forest (RF), a deep learning algorithm, and a combination thereof.

79. The non-transitory computer readable medium of claim 58, wherein (b) comprises comparing the data set to a reference data set.

80. The non-transitory computer readable medium of claim 79, wherein the reference data set comprises gene expression measurements of reference biological samples at each of the plurality of COVID-19 disease-associated genomic loci.

81. The non-transitory computer readable medium of claim 80, wherein the reference biological samples comprise a first plurality of biological samples obtained or derived from subjects having the COVID-19 disease and a second plurality of biological samples obtained or derived from subjects not having the COVID-19 disease.

82. The non-transitory computer readable medium of claim 58, wherein the biological sample is selected from the group consisting of: a blood sample, isolated peripheral blood mononuclear cells (PBMCs), a biopsy sample, and any derivative thereof.

83. The non-transitory computer readable medium of any one of claims 58-82, further comprising determining a likelihood of the determined COVID-19 disease state.

84. The non-transitory computer readable medium of any one of claims 58-83, further comprising monitoring the COVID-19 disease state of the subject, wherein the monitoring comprises assessing the COVID-19 disease state of the subject at a plurality of time points.

85. The non-transitory computer readable medium of claim 84, wherein a difference in the assessment of the COVID-19 disease state of the subject among the plurality of time points is indicative of one or more clinical indications selected from the group consisting of: (i) a diagnosis of the COVID-19 disease state of the subject, (ii) a prognosis of the COVID-19 disease state of the subject, and (iii) an efficacy or non-efficacy of a course of treatment for treating the COVID-19 disease state of the subject.
